(19) United States
(12) Patent Application Publication
(10) Pub. No.: US 2011/0151430 A1
(43) Pub. Date: Jun. 23, 2011

(54) VIRUS-SPECIFIC miRNA SIGNATURES FOR DIAGNOSIS AND THERAPEUTIC TREATMENT OF VIRAL INFECTION

(75) Inventors: Timothy F. KOWALIK, Princeton, MA (US); Bradford M. STADLER, Seattle, WA (US)

(73) Assignee: UNIVERSITY OF MASSACHUSETTS, Boston, MA (US)

(21) Appl. No.: 12/717,569

(22) Filed: Mar. 4, 2010

Related U.S. Application Data
(63) Continuation of application No. PCT/US2008/075646, filed on Sep. 8, 2008.
(60) Provisional application No. 60/967,780, filed on Sep. 6, 2007.

(51) Int. Cl. C12Q 1/70; C40B 40/06

(52) U.S. Cl. 435/5; 506/16

(57) ABSTRACT

The present invention related to miRNA signatures and diagnostic and therapeutic applications of miRNA signatures. The miRNA signatures are defined by a test sample miRNA profile relative to an appropriate control miRNA profile. In some embodiments, the test sample is a sample isolated, obtained or derived from a virus-infected cell or organism. The present invention further relates to the use of miRNA signatures in the identification of druggable targets and antiviral agents. Kits and compositions are also provided.

### RELATED APPLICATIONS

This application claims priority to PCT Application No. PCT/US2008/075646, filed on Sep. 8, 2008, and U.S. Provisional Patent Application No. 60/967,780, filed on Sep. 6, 2007. The entire contents of the foregoing patent applications are incorporated herein by reference.

### BACKGROUND OF THE INVENTION

RNAs that do not function as messenger RNAs, transfer RNAs or ribosomal RNAs are collectively termed non-coding RNAs (ncRNAs). ncRNAs can range in size from 21-25 nucleotides (nt) up to >10,000 nt, and estimates for the number of ncRNAs per genome range from hundreds to thousands. The functions of ncRNAs, although just beginning to be revealed, appear to vary widely from the purely structural to the purely regulatory, and include effects on transcription, translation, mRNA stability and chromatin structure (G Storz, Science (2002) 296:1260-1262). Two recent pivotal discoveries have placed ncRNAs in the spotlight: the identification of large numbers of very small ncRNAs of 20-24 nucleotides in length, termed micro RNAs (miRNAs), and the relationship of these miRNAs to intermediates in a eukaryotic RNA silencing mechanism known as RNA interference (RNAi).

RNA silencing refers to a group of sequence-specific, RNA-targeted gene-silencing mechanisms common to animals, plants, and some fungi, wherein RNA is used to target and destroy homologous mRNA, viral RNA, or other RNAs. RNA silencing was first observed in plants, where it was termed posttranscriptional gene silencing (PTGS). Researchers, trying to create more vividly purple flowers, introduced an extra copy of the gene conferring purple pigment. Surprisingly, the researchers discovered that the purple-conferring genes were switched off, or cosuppressed, producing white flowers. A similar phenomenon observed in Fungi was termed quelling. These phenomena were subsequently found to be related to a process in animals called RNA interference (RNAi). In RNAi, experimentally introduced double-stranded RNA (dsRNA) leads to loss of expression of the corresponding cellular gene. A key step in the molecular mechanism of RNAi is the processing of dsRNA by the ribonuclease Dicer into short dsRNAs, called small interfering RNAs (siRNAs), of ˜21-23 nt in length having specific features including 2 nt 3′-overhangs, a 5′-phosphate group and 3′-hydroxyl group. siRNAs are incorporated into a large nucleoprotein complex called an RNA-induced silencing complex (RISC). A distinct ribonuclease component of RISC uses the sequence encoded by the antisense strand of the siRNA as a guide to find and then cleave mRNAs of complementary sequence. The cleaved mRNA is ultimately degraded by cellular exonucleases. Thus, in PTGS, quelling, and RNAi, the silenced gene is transcribed normally into mRNA, but the mRNA is destroyed as quickly as it is made. In plants, it appears that PTGS evolved as a defense strategy against viral pathogens and transposons. While the introduction of long dsRNAs into plants and invertebrates initiates specific gene silencing (Hannon, 2002; Hutvagner, 2002), in mammalian cells, long dsRNA induces the potent translational inhibitory effects of the interferon response (Samuel, 2001). Short dsRNAs of <30 bp, however, evade the interferon response and are successfully incorporated into RISC to induce RNAi (Elbashir, 2001).

Another group of small ncRNAs, called micro RNAs (miRNAs), are related to the intermediates in RNAi and appear to be conserved from flies to humans (Lau, 2001; Lagos-Quintana, 2001; Rhoades, 2002). To date, all metazoans examined have been found to encode miRNAs. MicroRNAs are initially transcribed as a long, single-stranded miRNA precursor known as a pri-miRNA, which may contain one or several miRNAs, and these transcripts are then processed to ˜70 nt pre-miRNAs having a predicted stem-loop structure. The enzyme Dicer cleaves pre-miRNA to produce ˜20-25 nt miRNAs that function as single-stranded RNAi mediators capable of directing gene silencing (Hutvagner, 2002; McManus, 2002). These small transcripts have been proposed to play a role in development, apparently by suppressing target genes to which they have some degree of complementarity. The canonical miRNAs lin-4 and let-7 influence gene expression by binding to sequences of partial complementarity in the 3′ UTR of mRNA, thereby preventing mRNA translation (McCaffrey, 2002). In recent studies, however, miRNAs bearing perfect complementarity to a target RNA could function analogously to siRNAs, specifically directing degradation of the target sequences (Hutvagner, 2002b; Llave, 2002). Thus, the degree of complementarity between an miRNA and its target may determine whether the miRNA acts as a translational repressor or as a guide to induce mRNA cleavage. The discovery of miRNAs as endogenous small regulatory ncRNAs may represent the tip of an iceberg, as other groups of regulatory ncRNAs likely remain to be discovered.

Numerous recent studies have highlighted the importance of miRNAs in regulating gene expression. miRNAs can “fine-tune” gene expression by binding to nearly perfect complementary sequences in mRNAs, thus preventing their translation. The importance of miRNAs in the regulation of specific genes has been demonstrated in a variety of organisms, where their function impacts such universal cellular pathways as cell death, development, proliferation, and hematopoiesis (Ambros, 2004). Additionally, it has been demonstrated that several animal viruses encode their own miRNAs, which target either cellular or viral mRNAs (Cullen, 2006; Nair, 2006; Sarnow, 2006). Recent studies have further underscored the critical role of miRNAs in the maintenance of cellular homeostasis by demonstrating that miRNAs are misregulated in various forms of cancer. Furthermore, specific tumor types have been found to have specific patterns of miRNA expression, or “miRNA signatures” (Calin, 2006; Calin, 2002; Volinia, 2006; Yanaihara, 2006).

miRNA signatures characteristic of other pathological states have not been reported, and to date a comprehensive analysis of miRNA expression patterns during viral infection has not been conducted. The discovery of an miRNA signature characteristic of infection with a particular virus would help elucidate the role of specific cellular miRNAs and their corresponding target genes in viral replication and in the host response to infection. Such miRNA signatures could be used in, for example, diagnosis of infection with a particular viral species, determination of viral replication stage during a period of infection, and identification of druggable targets for the treatment of viral infection.

### SUMMARY OF THE INVENTION

The present invention is based in part on the discovery that infection with different viruses produces distinct patterns of miRNA expression, referred to herein as “miRNA signatures.” The instant inventors further discovered that distinct miRNAs are misregulated during human adenovirus or human cytomegalovirus (HCMV) infection, in the absence of widespread global changes in miRNA expression. Infection with adenovirus, a small DNA tumor virus, led to the upregulation of an oncogenic cluster of miRNAs that are misregulated in various tumors and have been shown to regulate expression of cell cycle genes. Expression of this cluster of miRNAs was not affected by infection with HCMV, a non-transforming virus. Based on these discoveries, miRNAs misregulated by specific viruses are believed to be important cellular mediators of viral infection.

Accordingly, the present invention features methods of identifying a virus-specific miRNA signature. Also featured are methods of identifying a virus-specific, replication stage-specific miRNA signature. Such signatures are further useful in drug discovery methodologies. The present invention also features methods for identifying druggable targets, in particular, antiviral drug targets, which are affected by miRNAs exhibiting an altered pattern of expression following viral infection. Also featured are methods of identifying antiviral agents which inhibit modulation of miRNA or mRNA expression by a virus. Also featured are methods of detecting a virus in a cell using a virus-specific miRNA signature. Further featured are methods of detecting the replication stage of a virus using a virus-specific, replication stage-specific miRNA signature. Also featured are methods of identifying an miRNA signature associated with a specific disease or pathological state of a subject infected with a virus. Also featured are methods of detecting the disease or pathological state of a subject infected with a virus using an miRNA signature associated with a specific disease or pathological state. Also featured are kits comprising microarrays with miRNA-specific probe oligonucleotides recognizing the miRNAs of an miRNA signature. Also featured are kits comprising sets of oligonucleotide primers designed to specifically amplify miRNAs of an miRNA signature.

Other features and advantages of the invention will be apparent from the following detailed description, and from the claims.

### BRIEF DESCRIPTION OF THE DRAWINGS

FIG. 1 graphically depicts differences in miRNA expression between mock-infected and Adenovirus-infected HeLa cells.

FIG. 2 depicts the expression level of miRNAs in Adenovirus-infected and mock-infected HeLa cells, where expression of the indicated miRNAs is reduced in Adenovirus-infected cells with respect to mock-infected cells.

FIG. 3 depicts the expression level of miRNAs in Adenovirus-infected and mock-infected HeLa cells, where expression of the indicated miRNAs is increased in Adenovirus-infected cells with respect to mock-infected cells.

FIG. 4 graphically depicts differences in miRNA expression between mock-infected and HCMV-infected HEL cells.

FIGS. 5a and 5b depict the expression level of miRNAs in HCMV-infected and mock-infected HEL cells, where expression of the indicated miRNAs is reduced in HCMV-infected cells with respect to mock-infected cells.

FIG. 6 depicts the expression level of miRNAs in HCMV-infected and mock-infected HEL cells, where expression of the indicated miRNAs is increased in HCMV-infected cells with respect to mock-infected cells.

FIG. 7a graphically depicts the hybridization signal intensities for individual miRNAs plotted as mock versus virus-infected. Signals generated for each of the six replicates of each miRNA are plotted. Open circles indicate statistically significant values (p<0.01) based on the average of 6 replicates/miRNA. Relevant changes in miRNA expression appear outside of the diagonal lines.

FIG. 7b depicts the number of statistically significant alterations in miRNA expression comprising the miRNA signatures for CMV and adenovirus. The two miRNA signatures are non-overlapping.

### DETAILED DESCRIPTION OF THE INVENTION

The present invention is based, at least in part, on the surprising discovery that infection with different viruses produces distinct patterns of cellular miRNA expression, or “miRNA signatures.” The present invention is based on the further discovery that, during human adenovirus or human cytomegalovirus infection, selected, distinct miRNAs are misregulated, in the absence of widespread alterations in global miRNA expression. Infection with adenovirus, a small DNA tumor virus, led to the upregulation of an oncogenic cluster of miRNAs that are misregulated in various tumors and have been shown to regulate expression of cell cycle genes. Expression of this cluster of miRNAs was not affected by infection with HCMV, a non-transforming virus. Infection with HCMV led to the upregulation and downregulation of a distinct population of miRNAs, the expression of which was not altered following infection with adenovirus. These findings indicate that miRNAs misregulated by specific viruses may be important cellular mediators of viral infection, or of the host response to the infection. Misregulation of cellular miRNAs by viruses indicates that the cellular or viral targets of misregulated miRNAs will likewise be regulated aberrantly during infection.

The concept that specific viruses distinctly modify cellular miRNA expression represents a hitherto unidentified mechanism by which viruses may control viral or cellular gene expression to produce an environment conducive to infection. Disruption of this viral function likely results in attenuation of viral infection, thus providing novel anti-viral approaches.

Cellular and/or viral genes or gene products whose expression is altered by misregulated miRNAs following viral infection make attractive targets for therapeutic anti-viral strategies. Such strategies include, for example, administration of a compound that inhibits or reduces expression of a gene or gene product that is expressed at elevated levels in virally infected cells compared with uninfected cells, wherein the elevated expression results from a virus-mediated reduction in expression of an miRNA targeting the gene. Such a compound may include, for example, an siRNA, an miRNA, a shRNA, or an antisense nucleic acid molecule. Said anti-viral strategies also include, for example, administration of a compound that increases expression of a gene or gene product that is expressed at reduced levels in virally infected cells compared with uninfected cells, wherein the reduced expression results from a virus-mediated increase in expression of an miRNA targeting the gene. Such a compound may include, for example, an expression vector or a recombinant protein.

Accordingly, the invention provides, in a first aspect, a method of identifying a druggable target, involving: (a) obtaining a cell or organism comprising an RNAi pathway; (b) infecting said cell or organism with a virus; and (c) assaying for expression of an miRNA; wherein a change in expression of an miRNA indicates that the miRNA is a druggable target.

In a related aspect, the invention provides a method of identifying a druggable target, involving: (a) obtaining a cell or organism comprising an RNAi pathway; (b) infecting said cell or organism with a virus; and (c) assaying for expression of an miRNA; wherein a change in expression of an miRNA indicates that an RNA targeted by the miRNA is a druggable target. In another embodiment, the gene or protein encoded by the targeted RNA is a druggable target. In a preferred embodiment, the targeted RNA is an mRNA, e.g., an mRNA that encodes a cellular protein or a viral protein. In an alternate embodiment, the targeted RNA is an ncRNA, e.g., a ncRNA that regulates gene expression.

In one embodiment of these aspects, the druggable target is an antiviral drug target. In another embodiment, the cell is a eukaryotic cell, e.g., a mammalian cell, a murine cell, an avian cell, a human cell and the like.

In one embodiment of these aspects, the change in expression of an miRNA is an increase in expression. In another embodiment, the change in expression of an miRNA is a decrease in expression. In one embodiment, the change in expression of an miRNA is measured using microarray analysis, northern blot analysis, in situ hybridization, or quantitative reverse transcriptase polymerase chain reaction.

In one embodiment of these aspects, the virus is capable of infecting eukaryotic cells, e.g., mammalian cells, avian cells, murine cells, human cells and the like. In various embodiments, the virus belongs to the Herpesviridae, Retroviridae, Reoviridae, Adenoviridae, Flaviviridae, Poxyiridae, Caliciviridae, Togaviridae, Coronaviridae, Rhabdoviridae, Filoviridae, Paramyxoviridae, Orthomyxoviridae, Bunyaviridae, Arenaviridae, Bornaviridae, Polyomaviridae, Papillomaviridae, Parvoviridae, Hepadnaviridae or Picornaviridae families. In a preferred embodiment, the virus is Adenovirus, Cytomegalovirus (e.g., HCMV), Epstein Barr virus (EBV), Human Papilloma virus (HPV), MHV-68, Human Immunodeficiency Virus (HIV), Hepatitis A Virus (HAV), Hepatitis B Virus (HBV), Hepatitis C Virus (HCV), Hepatitis E Virus (HEV), Rubella Virus, Mumps Virus, Measles Virus, Respiratory Syncytial Virus, Human T-cell Leukemia Virus, Lentivirus, Herpes Simplex Virus (e.g., Herpes Simplex 1 (HSV1), Herpes Simplex 2 (HSV2)), Varicella-Zoster Virus, Human Herpesviruses 6A, 6B, and 7, Kaposi's Sarcoma-Associated Herpesvirus (e.g., KSHV, HHV8), Cercopithecine Herpesvirus, Hepatitis Delta Virus, Dengue Virus, Foot and Mouth Disease Virus, Polyomavirus (e.g., JC, BK), Poliovirus, Coxsackievirus, Echovirus, Rhinovirus, Vacciniavirus, Small Pox Virus, Influenza Virus, or Avian Influenza Virus. In an exemplary embodiment, the virus is Ad-5 or Ad-169.

In a related aspect, the invention provides a druggable target, e.g. an antiviral drug target, identified according to the provided methods of the invention. Such antiviral drug targets are useful in methods for identifying an antiviral agent, e.g., methods that involve assaying a test agent for activity against the antiviral drug target. In preferred embodiments, a method for identifying an antiviral agent involves assaying a test agent for the ability to enhance or inhibit the expression or activity of the antiviral drug target.

The invention provides, in another aspect, a method for identifying an antiviral agent, involving: (a) contacting a cell with a test agent, said cell comprising an RNAi pathway and a virus, wherein said virus modulates the expression of one or more cellular miRNAs; (b) assaying for a change in expression of said miRNAs; and (c) identifying a test agent based on its ability to inhibit modulation of cellular miRNA expression by the virus. In one embodiment of this aspect, the virus increases the expression of one or more cellular miRNAs. In a related embodiment, the test agent is identified based on its ability to inhibit virus-mediated increases in miRNA expression. In another embodiment, the virus decreases the expression of one or more cellular miRNAs. In a related embodiment, the test agent is identified based on its ability to inhibit virus-mediated decreases in miRNA expression.

In another embodiment of this aspect, the virus is capable of infecting eukaryotic cells, e.g., mammalian cells. In various embodiments, the virus belongs to the Herpesviridae, Retroviridae, Reoviridae, Adenoviridae, Flaviviridae, Poxyiridae, Caliciviridae, Togaviridae, Coronaviridae, Rhabdoviridae, Filoviridae, Paramyxoviridae, Orthomyxoviridae, Bunyaviridae, Arenaviridae, Bornaviridae, Polyomaviridae, Papillomaviridae, Parvoviridae, Hepadnaviridae or Picornaviridae families. In a preferred embodiment, the virus is Adenovirus, Cytomegalovirus (e.g., HCMV), Epstein Barr virus (EBV), Human Papilloma virus (HPV), MHV-68, Human Immunodeficiency Virus (HIV), Hepatitis A Virus (HAV), Hepatitis B Virus (HBV), Hepatitis C Virus (HCV), Hepatitis E Virus (HEV), Rubella Virus, Mumps Virus, Measles Virus, Respiratory Syncytial Virus, Human T-cell Leukemia Virus, Lentivirus, Herpes Simplex Virus (e.g., Herpes Simplex 1 (HSV1), Herpes Simplex 2 (HSV2)), Varicella-Zoster Virus, Human Herpesviruses 6A, 6B, and 7, Kaposi's Sarcoma-Associated Herpesvirus (e.g., KSHV, HHV8), Cercopithecine Herpesvirus, Hepatitis Delta Virus, Dengue Virus, Foot and Mouth Disease Virus, Polyomavirus (e.g., JC, BK), Poliovirus, Coxsackievirus, Echovirus, Rhinovirus, Vacciniavirus, Small Pox Virus, Influenza Virus, or Avian Influenza Virus. In an exemplary embodiment, the virus is Ad-5 or Ad-169.

In a further embodiment of this aspect, the miRNAs may include miRNAs of the eukaryotic miRNome. In one embodiment, the miRNAs may include miRNAs selected from those listed in Tables 1-3. In various embodiments, the miRNAs may include hsa-miR-34c*, hsa-miR-19b***, hsa-miR-19a***, hsa-miR-186, hsa-miR-17-5p***, hsa-miR-147*, hsa-miR-135b*, hsa-miR-132, hsa-miR-98, hsa-miR-505*, hsa-miR-423, hsa-miR-324-3p, hsa-miR-30a-5p, hsa-miR-28, hsa-miR-25, hsa-miR-224, hsa-miR-193b*, hsa-miR-181b, hsa-let-71, hsa-miR-126, hsa-miR-134, hsa-miR-135b*, hsa-miR-146a, hsa-miR-182, hsa-miR-183, hsa-miR-194, hsa-miR-199b*, hsa-miR-212*, hsa-miR-215, hsa-miR-34c*, hsa-miR-361*, hsa-miR-362, hsa-miR-422b, hsa-miR-500, hsa-miR-500*, hsa-miR-502*, hsa-miR-532, hsa-miR-660, hsa-miR-100, hsa-miR-10a, hsa-miR-10a*, hsa-miR-125b, hsa-miR-127, hsa-miR-143, hsa-miR-145, hsa-miR-145*, hsa-miR-148b, hsa-miR-152, hsa-miR-154, hsa-miR-155, hsa-miR-181a, hsa-miR-181b, hsa-miR-181c, hsa-miR-181d, hsa-miR-193a*, hsa-miR-199a, hsa-miR-199a*, hsa-miR-199b, hsa-miR-214, hsa-miR-218, hsa-miR-221*, hsa-miR-222, hsa-miR-29b-1*, hsa-miR-29b-2*, hsa-miR-335, hsa-miR-34a, hsa-miR-379*, hsa-miR-409-3p, hsa-miR-409-5p, hsa-miR-410, hsa-miR-411, hsa-miR-421, hsa-miR-424, hsa-miR-424*, hsa-miR-432, hsa-miR-450, hsa-miR-455, hsa-miR-484, hsa-miR-485-5p, hsa-miR-487b, hsa-miR-490*, hsa-miR-493-3p, hsa-miR-493-5p, hsa-miR-494, hsa-miR-495, hsa-miR-503, hsa-miR-505, hsa-miR-542-3p, hsa-miR-542-5p, hsa-miR-574, hsa-miR-594, hsa-miR-7, or hsa-miR-99a.

The invention also provides an agent that is identified according to the methods of these aspects, as well as a pharmaceutical composition comprising the agent and a pharmaceutically acceptable carrier. These agents and compositions can be administered in an effective dose to an organism or subject in methods for attenuating and/or treating a viral infection. Preferably, the organism is a eukaryotic organism, e.g., a mammal, e.g., a human.

The invention further features miRNA signatures that are characteristic of infection with a distinct type of virus. These signatures have utility in identifying factors essential for replication or establishment of latency/chronic infection by the virus. The individual miRNA components of these signatures, and the genes and encoded proteins that these miRNAs target, represent a pool of cellular or viral targets for therapeutic intervention in the treatment of viral infection. These specifically defined miRNA signatures also have utility in diagnostic applications, e.g., in detecting the presence of a virus in a cell, in detecting the replication stage of a virus in a cell, in diagnosing a viral infection in an organism, e.g., a human, or in identifying a specific disease or pathological state of a subject infected with a virus.

Accordingly, in another aspect, the invention provides a method of identifying a virus-specific signature, involving (a) generating a miRNA profile from a test sample obtained from a cell infected with a virus; and (b) comparing the test sample a miRNA profile to an appropriate control sample miRNA profile; wherein one or more alterations between the test sample miRNA profile and the control sample miRNA profile defines the virus-specific miRNA signature.

In one embodiment, this method involves (a) labeling miRNAs isolated from a test sample obtained from a cell infected with a virus to provide a set of target oligodeoxynucleotides; (b) hybridizing the target oligodeoxynucleotides to a microarray comprising miRNA-specific probe oligonucleotides to provide a hybridization signal profile for the test sample; and (c) comparing the test sample hybridization signal profile to a hybridization signal profile generated from a control sample; wherein a set of one or more alterations between the hybridization signal of the test sample and the hybridization signal of the control sample is identified as the virus-specific miRNA signature.

In a related aspect, the invention provides a method of identifying a virus-specific, replication stage-specific signature, involving (a) generating a miRNA profile from a test sample obtained from a cell infected with a virus, wherein said virus is in a replication stage selected from the group consisting of productive, persistent and latent; and (b) comparing the test sample a miRNA profile to an appropriate control sample miRNA profile; wherein one or more alterations between the test sample a miRNA profile and the control sample miRNA profile defines the virus-specific, replication stage-specific miRNA signature.

In one embodiment, this method involves (a) labeling miRNAs isolated from a test sample obtained from a cell infected with a virus to provide a set of target oligodeoxynucleotides;, wherein said virus is in a replication stage selected from the group consisting of productive, persistent, latent, early, immediate-early, late, active and chronic; (b) hybridizing the target oligodeoxynucleotides to a microarray comprising miRNA-specific probe oligonucleotides to provide a hybridization signal profile for the test sample; and (c) comparing the test sample hybridization signal profile to a hybridization signal profile generated from a control sample; wherein a set of one or more alterations between the hybridization signal profile of the test sample and the hybridization signal profile of the control sample is identified as the virus-specific, replication stage-specific miRNA signature.

In various embodiments of these aspects, the cell infected with a virus is a eukaryotic cell, e.g., a mammalian cell, a murine cell, an avian cell, or a human cell. In one embodiment, the cell is obtained from an organism infected with the virus.

In another embodiment of these aspects, the cell is obtained from a subject having a high likelihood of having a viral infection. In certain embodiments, the subject is an organ transplant recipient. Organ transplant recipients are frequently administered immunosuppressive therapy, and are at increased risk of developing a viral infection, including a viral infection acquired from a virus present in the allograft. miRNA signatures serve as biomarkers of viral infection, treatment, and recovery in patients following organ transplantation. In various embodiments, the transplanted organ is a tissue, an organ, or a portion of thereof. In exemplary embodiments, the transplanted organ is kidney, liver, heart, lung, or skin. miRNA profiles may be determined in a sample obtained from a subject prior to transplantation or following transplantation, either prior to infection, during active infection, or during various stages of treatment and recovery from infection. These miRNA profiles may be used to determine miRNA signatures characteristic of infection with a particular virus following organ transplantation. In certain embodiments, the virus is CMV. In various embodiments of these aspects, the microarray includes miRNA specific probe oligonucleotides recognizing all or part of the miRNome of a given species. In preferred embodiments, the probe oligonucleotides recognize all or part of the human miRNome, the murine miRNome, or the avian miRNome.

In various embodiments of these aspects, the control sample is obtained from a cell that has not been infected with the same virus as the test sample. In preferred embodiments, the control sample is obtained from an uninfected cell or a mock infected cell. In a particularly preferred embodiment, the control sample is obtained from an uninfected cell or a mock infected cell of the same species from which the test sample is derived.

In another embodiment of these aspects, the virus is capable of infecting eukaryotic cells, e.g., mammalian cells. In various embodiments, the virus belongs to the Herpesviridae, Retroviridae, Reoviridae, Adenoviridae, Flaviviridae, Poxyiridae, Caliciviridae, Togaviridae, Coronaviridae, Rhabdoviridae, Filoviridae, Paramyxoviridae, Orthomyxoviridae, Bunyaviridae, Arenaviridae, Bornaviridae, Polyomaviridae, Papillomaviridae, Parvoviridae, Hepadnaviridae or Picornaviridae families. In a preferred embodiment, the virus is Adenovirus, Cytomegalovirus (e.g., HCMV), Epstein Barr virus (EBV), Human Papilloma virus (HPV), MHV-68, Human Immunodeficiency Virus (HIV), Hepatitis A Virus (HAV), Hepatitis B Virus (HBV), Hepatitis C Virus (HCV), Hepatitis E Virus (HEV), Rubella Virus, Mumps Virus, Measles Virus, Respiratory Syncytial Virus, Human T-cell Leukemia Virus, Lentivirus, Herpes Simplex Virus (e.g., Herpes Simplex 1 (HSV1), Herpes Simplex 2 (HSV2)), Varicella-Zoster Virus, Human Herpesviruses 6A, 6B, and 7, Kaposi's Sarcoma-Associated Herpesvirus (e.g., KSHV, HHV8), Cercopithecine Herpesvirus, Hepatitis Delta Virus, Dengue Virus, Foot and Mouth Disease Virus, Polyomavirus (e.g., JC, BK), Poliovirus, Coxsackievirus, Echovirus, Rhinovirus, Vacciniavirus, Small Pox Virus, Influenza Virus, or Avian Influenza Virus. In an exemplary embodiment, the virus is Ad-5 or Ad-169.

In a further embodiment of these aspects, the miRNA signature may include miRNAs of the eukaryotic miRNome. In a preferred embodiment, the miRNA signature includes hsa-miR-34c*, hsa-miR-19b***, hsa-miR-19a***, hsa-miR-186, hsa-miR-17-5p***, hsa-miR-147*, hsa-miR-135b*, hsa-miR-132, hsa-miR-98, hsa-miR-505*, hsa-miR-423, hsa-miR-324-3p, hsa-miR-30a-5p, hsa-miR-28, hsa-miR-25, hsa-miR-224, hsa-miR-193b*, hsa-miR-181b, and hsa-let-71. In another preferred embodiment, the miRNA signature includes hsa-miR-126, hsa-miR-134, hsa-miR-135b*, hsa-miR-146a, hsa-miR-182, hsa-miR-183, hsa-miR-194, hsa-miR-199b*, hsa-miR-212*, hsa-miR-215, hsa-miR-34c*, hsa-miR-361*, hsa-miR-362, hsa-miR-422b, hsa-miR-500, hsa-miR-500*, hsa-miR-502*, hsa-miR-532, hsa-miR-660, hsa-miR-100, hsa-miR-10a, hsa-miR-10a*, hsa-miR-125b, hsa-miR-127, hsa-miR-143, hsa-miR-145, hsa-miR-145*, hsa-miR-148b, hsa-miR-152, hsa-miR-154, hsa-miR-155, hsa-miR-181a, hsa-miR-181b, hsa-miR-181c, hsa-miR-181d, hsa-miR-193a*, hsa-miR-199a, hsa-miR-199a*, hsa-miR-199b, hsa-miR-214, hsa-miR-218, hsa-miR-221*, hsa-miR-222, hsa-miR-29b-1*, hsa-miR-29b-2*, hsa-miR-335, hsa-miR-34a, hsa-miR-379*, hsa-miR-409-3p, hsa-miR-409-5p, hsa-miR-410, hsa-miR-411, hsa-miR-421, hsa-miR-424, hsa-miR-424*, hsa-miR-432, hsa-miR-450, hsa-miR-455, hsa-miR-484, hsa-miR-485-5p, hsa-miR-487b, hsa-miR-490*, hsa-miR-493-3p, hsa-miR-493-5p, hsa-miR-494, hsa-miR-495, hsa-miR-503, hsa-miR-505, hsa-miR-542-3p, hsa-miR-542-5p, hsa-miR-574, hsa-miR-594, hsa-miR-7, and hsa-miR-99a.

In another aspect, the invention provides a method of detecting the presence of a virus in a biological sample, comprising determining an miRNA profile for the sample and comparing the miRNA profile to a previously identified virus-specific miRNA signature, wherein a substantial similarity in said profiles indicates the presence of the virus in the sample. In a related aspect, the invention provides a method of detecting the presence of a virus in a biological sample, comprising measuring in the sample the level of at least one miRNA associated with a virus-specific miRNA signature, wherein an alteration in the level of the miRNA in the sample relative to the level of corresponding miRNA in a control sample is indicative of the sample containing the virus.

In another aspect, the invention provides a method of detecting the replication stage of a virus in a biological sample, comprising determining a miRNA profile for the sample and comparing the miRNA profile to a previously identified virus-specific, replication stage-specific miRNA signature, wherein a substantial similarity in said profiles indicates the replication stage of a virus in the sample. In a related aspect, the invention provides a method of detecting the replication stage of a virus in a biological sample, comprising measuring in the sample the level of at least one miRNA associated with a virus-specific replication stage-specific miRNA signature, wherein an alteration in the level of the miRNA in the sample relative to the level of corresponding miRNA in a control sample is indicative of the replication stage of a virus in the sample.

In one embodiment of these aspects, the virus-specific and replication stage-specific miRNA signatures are identified using methods of the instant invention. In another embodiment, the cell is a eukaryotic cell, e.g., a mammalian cell, a murine cell, an avian cell, or a human cell. In a preferred embodiment, the cell is obtained from a sample obtained from a subject, e.g., a biopsy sample, a blood sample, or another fluid sample. In one embodiment, the cell is obtained from a subject following organ transplantation. In an exemplary embodiment, the transplanted organ is kidney, liver, heart, lung or skin.

In another embodiment, the virus is capable of infecting eukaryotic cells, e.g., mammalian cells. In various embodiments, the virus belongs to the Herpesviridae, Retroviridae, Reoviridae, Adenoviridae, Flaviviridae, Poxyiridae, Caliciviridae, Togaviridae, Coronaviridae, Rhabdoviridae, Filoviridae, Paramyxoviridae, Orthomyxoviridae, Bunyaviridae, Arenaviridae, Bornaviridae, Polyomaviridae, Papillomaviridae, Parvoviridae, Hepadnaviridae or Picornaviridae families. In a preferred embodiment, the virus is Adenovirus, Cytomegalovirus (e.g., HCMV), Epstein Barr virus (EBV), Human Papilloma virus (HPV), MHV-68, Human Immunodeficiency Virus (HIV), Hepatitis A Virus (HAV), Hepatitis B Virus (HBV), Hepatitis C Virus (HCV), Hepatitis E Virus (HEV), Rubella Virus, Mumps Virus, Measles Virus, Respiratory Syncytial Virus, Human T-cell Leukemia Virus, Lentivirus, Herpes Simplex Virus (e.g., Herpes Simplex 1 (HSV1), Herpes Simplex 2 (HSV2)), Varicella-Zoster Virus, Human Herpesviruses 6A, 6B, and 7, Kaposi's Sarcoma-Associated Herpesvirus (e.g., KSHV, HHV8), Cercopithecine Herpesvirus, Hepatitis Delta Virus, Dengue Virus, Foot and Mouth Disease Virus, Polyomavirus (e.g., JC, BK), Poliovirus, Coxsackievirus, Echovirus, Rhinovirus, Vacciniavirus, Small Pox Virus, Influenza Virus, or Avian Influenza Virus.

In another aspect, the invention provides a method of identifying an miRNA signature associated with a specific disease or pathological state of a subject infected with a virus, involving (a) generating a miRNA profile from a test sample obtained from the subject; and (b) comparing the test sample a miRNA profile to an appropriate control sample miRNA profile; wherein one or more alterations between the test sample miRNA profile and the control sample miRNA profile defines the miRNA signature associated with the disease or pathological state.

In a related aspect, the invention provides a method of detecting a disease or pathological state of a subject infected with a virus, involving determining an miRNA profile for a sample obtained from the subject, and comparing the miRNA profile to a previously identified miRNA signature specific to a given disease or pathological state, wherein a substantial similarity in said profiles indicates the disease or pathological state of the subject. In another aspect the invention provides a method of detecting a disease or pathological state of a subject infected with a virus, comprising measuring in the sample the level of at least one miRNA associated with a given disease or pathological state, wherein an alteration in the level of the miRNA in the sample relative to the level of corresponding miRNA in a control sample is indicative of the disease or pathological state of the subject.

In one embodiment of these aspects, the miRNA signature is identified using methods of the instant invention.

In one embodiment of these aspects, the subject is a subject infected with a virus.

In another embodiment, the subject is an organ transplant recipient. In exemplary embodiments, the transplanted organ is kidney, liver, heart or lung.

In various embodiments of these aspects, the microarray includes miRNA specific probe oligonucleotides recognizing all or part of the miRNome of a given species. In preferred embodiments, the probe oligonucleotides recognize all or part of the human miRNome and/or a viral miRNome.

In various embodiments of these aspects, the control sample is obtained from a cell or subject that has not been infected with the same virus as the test subject.

In another embodiment of these aspects, the virus is capable of infecting eukaryotic cells, e.g., mammalian cells. In various embodiments, the virus belongs to the Herpesviridae, Retroviridae, Reoviridae, Adenoviridae, Flaviviridae, Poxyiridae, Caliciviridae, Togaviridae, Coronaviridae, Rhabdoviridae, Filoviridae, Paramyxoviridae, Orthomyxoviridae, Bunyaviridae, Arenaviridae, Bornaviridae, Polyomaviridae, Papillomaviridae, Parvoviridae, Hepadnaviridae or Picornaviridae families. In a preferred embodiment, the virus is Adenovirus, Cytomegalovirus (e.g., HCMV), Epstein Barr virus (EBV), Human Papilloma virus (HPV), MHV-68, Human Immunodeficiency Virus (HIV), Hepatitis A Virus (HAV), Hepatitis B Virus (HBV), Hepatitis C Virus (HCV), Hepatitis E Virus (HEV), Rubella Virus, Mumps Virus, Measles Virus, Respiratory Syncytial Virus, Human T-cell Leukemia Virus, Lentivirus, Herpes Simplex Virus (e.g., Herpes Simplex 1 (HSV1), Herpes Simplex 2 (HSV2)), Varicella-Zoster Virus, Human Herpesviruses 6A, 6B, and 7, Kaposi's Sarcoma-Associated Herpesvirus (e.g., KSHV, HHV8), Cercopithecine Herpesvirus, Hepatitis Delta Virus, Dengue Virus, Foot and Mouth Disease Virus, Polyomavirus (e.g., JC, BK), Poliovirus, Coxsackievirus, Echovirus, Rhinovirus, Vacciniavirus, Small Pox Virus, Influenza Virus, or Avian Influenza Virus. In an exemplary embodiment, the virus is Ad-5 or Ad-169.

In a further embodiment of these aspects, the miRNA signature may include miRNAs of the eukaryotic miRNome. In a preferred embodiment, the miRNA signature includes hsa-miR-34c*, hsa-miR-19b***, hsa-miR-19a***, hsa-miR-186, hsa-miR-17-5p***, hsa-miR-147*, hsa-miR-135b*, hsa-miR-132, hsa-miR-98, hsa-miR-505*, hsa-miR-423, hsa-miR-324-3p, hsa-miR-30a-5p, hsa-miR-28, hsa-miR-25, hsa-miR-224, hsa-miR-193b*, hsa-miR-181b, and hsa-let-71. In another preferred embodiment, the miRNA signature includes hsa-miR-126, hsa-miR-134, hsa-miR-135b*, hsa-miR-146a, hsa-miR-182, hsa-miR-183, hsa-miR-194, hsa-miR-199b*, hsa-miR-212*, hsa-miR-215, hsa-miR-34c*, hsa-miR-361*, hsa-miR-362, hsa-miR-422b, hsa-miR-500, hsa-miR-500*, hsa-miR-502*, hsa-miR-532, hsa-miR-660, hsa-miR-100, hsa-miR-10a, hsa-miR-10a*, hsa-miR-125b, hsa-miR-127, hsa-miR-143, hsa-miR-145, hsa-miR-145*, hsa-miR-148b, hsa-miR-152, hsa-miR-154, hsa-miR-155, hsa-miR-181a, hsa-miR-181b, hsa-miR-181c, hsa-miR-181d, hsa-miR-193a*, hsa-miR-199a, hsa-miR-199a*, hsa-miR-199b, hsa-miR-214, hsa-miR-218, hsa-miR-221*, hsa-miR-222, hsa-miR-29b-1*, hsa-miR-29b-2*, hsa-miR-335, hsa-miR-34a, hsa-miR-379*, hsa-miR-409-3p, hsa-miR-409-5p, hsa-miR-410, hsa-miR-411, hsa-miR-421, hsa-miR-424, hsa-miR-424*, hsa-miR-432, hsa-miR-450, hsa-miR-455, hsa-miR-484, hsa-miR-485-5p, hsa-miR-487b, hsa-miR-490*, hsa-miR-493-3p, hsa-miR-493-5p, hsa-miR-494, hsa-miR-495, hsa-miR-503, hsa-miR-505, hsa-miR-542-3p, hsa-miR-542-5p, hsa-miR-574, hsa-miR-594, hsa-miR-7, and hsa-miR-99a.

In another aspect, the invention provides microarrays that include miRNA-specific probe oligonucleotides that recognize the miRNAs of an miRNA signature identified according to the methods of the instant invention. In yet another aspect, the invention provides sets of oligonucleotide primers designed to specifically amplify the miRNAs of an miRNA signature identified according to the methods of the instant invention in a quantitative reverse-transcription polymerase chain reaction.

In another aspect, the invention provides kits that include a microarray containing miRNA-specific probe oligonucleotides that recognize the miRNAs of an miRNA signature identified according to the methods of the instant invention, and instructions for use. In yet another aspect, the invention provides kits that include sets of oligonucleotide primers designed to specifically amplify the miRNAs of an miRNA signature identified according to the methods of the instant invention in a quantitative reverse-transcription polymerase chain reaction assay, and instructions for use.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### I. DEFINITIONS

So that the invention may be more readily understood, certain terms are first defined.

The term “target gene”, as used herein, refers to a gene or gene product intended for downregulation via RNA interference (“RNAi”). The term “target protein” refers to a protein intended for downregulation via RNAi. The term “target RNA” refers to an RNA molecule intended for degradation by RNAi. The term “target RNA” includes both non-coding RNA molecules (transcribed from a DNA but not encoding polypeptide sequence) and coding RNA molecules (i.e., mRNA molecules). A “target RNA” is also referred to herein as a “transcript”.

The term “RNA interference” or “RNAi”, as used herein, refers generally to a sequence-specific or selective process by which a target molecule (e.g., a target gene, protein or RNA) is downregulated. In specific embodiments, the process of “RNA interference” or “RNAi” features degradation of RNA molecules, e.g., RNA molecules within a cell, said degradation being triggered by an RNA agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

The term “RNA agent”, as used herein, refers to an RNA (or analog thereof), having sufficient sequence complimentarity to a target RNA (i.e., the RNA being degraded) to direct RNAi. A RNA agent having a “sequence sufficiently complementary to a target RNA sequence to direct RNAi” means that the RNA agent has a sequence sufficient to trigger the destruction of the target RNA by the RNAi machinery (e.g., the RISC complex) or process.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double-stranded, i.e., dsRNA and dsDNA, respectively).

The term RNA includes noncoding (“ncRNAs”) and coding RNAs (i.e., mRNAs, as defined herein). ncRNAs are single- or double-stranded RNAs that do not specify the amino acid sequence of polypeptides (i.e., do not encode polypeptides). By contrast, ncRNAs affect processes including, but not limited to, transcription, gene silencing, replication, RNA processing, RNA modification, RNA stability, mRNA translation, protein stability, and/or protein translation. ncRNAs include, but are not limited to, bacterial small RNAs (“sRNA”), microRNAs (“miRNAs”), and/or small temporal RNAs (“stRNAs”).

The term “mRNA” or “messenger RNA” refers to a single-stranded RNA that specifies the amino acid sequence of one or more polypeptide chains. This information is translated during protein synthesis when ribosomes bind to the mRNA.

The term “transcript” refers to a RNA molecule transcribed from a DNA or RNA template by a RNA polymerase template. The term “transcript” includes RNAs that encode polypeptides (i.e., mRNAs) as well as noncoding RNAs (“ncRNAs”).

As used herein, expression of an RNA (e.g., an mRNA, an miRNA, an ncRNA) is “upregulated” when the amount of RNA gene product present in a cell or biological sample is greater than the amount of RNA gene product present in a control cell or biological sample. Likewise, expression of an RNA is “downregulated” when the amount of RNA gene product present in a cell or biological sample is less than the amount of RNA gene product present in a control cell or biological sample.

As used herein, the term “small interfering RNA” (“siRNA”) (also referred to in the art as “short interfering RNAs”) refers to an RNA agent, preferably a double-stranded agent, of about 10-50 nucleotides in length (the term “nucleotides” including nucleotide analogs), preferably between about 15-25 nucleotides in length, more preferably about 17, 18, 19, 20, 21, 22, 23, 24, or 25 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof).

As used herein, the term “miRNA” or “microRNA” refers to an RNA agent, preferably a single-stranded agent, of about 10-50 nucleotides in length (the term “nucleotides” including nucleotide analogs), preferably between about 15-25 nucleotides in length, more preferably about 17, 18, 19, 20, 21, 22, 23, 24, or 25 nucleotides in length, which is capable of directing or mediating RNA interference. Naturally-occurring miRNAs are generated from stem-loop precursor RNAs (i.e., pre-miRNAs) by Dicer. The term “Dicer” as used herein, includes Dicer as well as any Dicer orthologue or homologue capable of processing dsRNA structures into siRNAs, miRNAs, siRNA-like or miRNA-like molecules. The term microRNA (or “miRNA”) is used interchangeably with the term “small temporal RNA” (or “stRNA”) based on the fact that naturally-occurring microRNAs (or “miRNAs”) have been found to be expressed in a temporal fashion (e.g., during development).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. shRNAs may be substrates for the enzyme Dicer, and the products of Dicer cleavage may participate in RNAi. shRNAs may be derived from transcription of an endogenous gene encoding a shRNA, or may be derived from transcription of an exogenous gene introduced into a cell or organism on a vector, e.g., a plasmid vector or a viral vector. An exogenous gene encoding an shRNA can additionally be introduced into a cell or organism using other methods known in the art, e.g., lipofection, nucleofection, etc.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

The term “oligonucleotide” refers to a short polymer of nucleotides and/or nucleotide analogs. The term “RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Preferred RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, the term “isolated RNA” (e.g., “isolated mRNA”, “isolated miRNA” or “isolated RNAi agent”) refers to RNA molecules which are substantially free of other cellular material, or culture medium when produced by recombinant techniques, or substantially free of chemical precursors or other chemicals when chemically synthesized.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “druggable target” refers to a target (i.e, gene or gene product) having certain desired properties which indicate a potential for drug discovery, i.e., for use in the identification, research and/or development of therapeutically relevant compounds. A druggable target is distinguished based on certain physical and/or functional properties selected by a person skilled in the art of drug discovery. A druggable target (i.e., gene or gene product) of the instant invention, for example, is distinguished from other genes and/or gene products based on the fact that that it is regulated following viral infection.

Based on the fact that these targets are regulated by viral infection, it is believed that the targets are important in essential cellular processes, for example, maintenance of cellular homeostasis, host cell defense mechanisms, and the like. Control of such processes, including situations in which such processes are misregulated (i.e., in the biology of a disease), has obvious therapeutic appeal. Additional criteria for identifying and/or selecting druggable targets include, but are not limited to (1) cellular localization susceptible to systemically administered (e.g., orally administered) drugs; (2) homology or similarity to other genes and/or gene products (e.g., members of a gene family) previously successfully targeted; and (3) data (e.g., expression and/or activity data) indicating a role for the gene/gene product at a critical intervention points in a disease pathway.

The term “antiviral drug target”, as used herein, refers to a target (i.e, gene or gene product) having certain desired properties which indicate a potential for antivral drug discovery, i.e., for use in the identification, research and/or development of compounds useful in antiviral therapies. A druggable target (i.e., gene or gene product) of the instant invention, for example, is indicated as an antiviral drug target based on the fact that viral RNAs, in particular, svRNAs, VA RNAs, or derivatives thereof can act as mediators (e.g., substrates and/or inhibitors) of RNAi.

A gene “involved” in a disorder includes a gene, the normal or aberrant expression or function of which effects or causes a disease or disorder or at least one symptom of said disease or disorder.

The phrase “examining the function of a gene in a cell or organism” refers to examining or studying the expression, activity, function or phenotype arising therefrom. Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNAi agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

As used herein, the term “miRNA profile” refers to a specific pattern of detectable signals indicative of miRNA expression in a sample. In one embodiment, the detectable signals are nucleic acid hybridization signals, for example, signals generated by hybridization of miRNAs in the sample to miRNA nucleic acid probes, e.g. probes having sequence complementarity to the miRNAs. Exemplary detectable labels include, but are not limited to, radioactive labels, fluorescent labels probes, colorometric labels, biotin labels, etc. Probes and/or miRNAs can be immobilized, for example, on a chip, membrane, slide, film, etc. In other embodiments, hybridization can be accomplished with one or more components in solution. In preferred aspects of the invention, a miRNA profile consists of a plurality of signals of varied intensity, the pattern of which is reproducible when detected in replicate samples.

As used herein, the term “miRNA signature” refers to a test sample miRNA profile relative to an appropriate control miRNA profile. In preferred embodiments, the “test sample” is a sample isolated, obtained or derived from a treated or manipulated cell or organism. In particularly preferred embodiments, the “test sample” is a sample isolated, obtained or derived from a virus-infected cell or organism.

In some embodiments, the miRNA signature is associated with a specific cellular condition. In some embodiments, the miRNA signature features or consists essentially of miRNAs that are coordinately regulated. These miRNAs may be expressed in a specific cell lineage, stage of differentiation, or during a particular biological response (e.g., in response to viral infection, in response to a particular replication stage of a virus following viral infection, in response to a change in disease or pathological state following viral infection, etc.).

As used herein, the term “miRNome” refers to the miRNA gene complement of the genome of a given organism, i.e., the full cellular complement of miRNA. The miRNome of an organism is continually being updated as new miRNA sequences are identified in the art. In preferred embodiments, miRNAs comprising the human miRNome, the murine miRNome, the avian miRNome, and the viral miRNome are used in the methods of the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Various aspects of the invention are described in further detail in the following subsections.

### II. miRNAs AND RNA INTERFERENCE

MicroRNAs (miRNAs) are small (e.g., 17-25 nucleotides), single-stranded noncoding RNA molecules that regulate gene expression in eukaryotes at the level of translation. MicroRNAs are initially transcribed as a long, single-stranded miRNA precursor known as a pri-miRNA, which may contain one or several miRNAs. These pri-miRNAs typically contain regions of localized stem-loop hairpin structures that contain the mature miRNA sequences. Pri-miRNAs are processed into 70-100 nucleotide pre-miRNAs in the nucleus by the double-stranded RNA-specific nuclease Drosha. These 70-100 nucleotide pre-miRNAs are transported to the cytoplasm, where they are processed by the enzyme Dicer into single-stranded mature miRNAs of about 19-25 nucleotides. This is in contrast with siRNAs, which are of a similar size but are double-stranded, and are usually processed from a double-stranded RNA precursor. Following processing, mature miRNAs are incorporated into a RISC(RNA-Induced Silencing Complex), which participates in RNA interference (RNAi). miRNAs can pair with target mRNAs that contain sequences only partially complementary (e.g., 30%, 35%, 40%, 50%, 55%, 60%, 65%, 70%, 75%, 80% or more) to the miRNA. Such pairing results in repression of mRNA translation without altering mRNA stability. Alternatively, miRNAs with a substantial degree of complementarity to their targets effect gene silencing by mediating mRNA degradation (Hutvágner and Zamore (2002) Science 297:2056-2060). As expression of precursor microRNAs (i.e., pri-miRNAs) is often developmentally regulated, miRNAs are often referred to interchangeably in the art as “small temporal RNAs” or “stRNAs”.

C. elegans contains approximately 100 endogenous miRNA genes, about 30% of which are conserved in vertebrates. Mammalian genomes are predicted to encode at least 200 to 1000 distinct miRNAs, many of which are estimated to interact with 5-10 different mRNA transcripts. Accordingly, miRNAs are predicted to regulate up to one-third of all genes. miRNAs are differentially expressed in various tissues, such that each tissue is characterized by a specific set of miRNAs. miRNAs have been shown to be important modulators of cellular pathways including growth and proliferation, apoptosis, and developmental timing. Given the pathways over which miRNAs exert a regulatory effect, it is not surprising that alterations in miRNA expression have been detected in several types of cancer, including breast and lung carcinomas. These recognized pathways likely represent the tip of an iceberg, however, as the abundance of miRNAs within eukaryotic cells indicates that many downstream effects of miRNA-induced silencing remain to be identified.

### III. EXPRESSION OF VIRALLY-DERIVED miRNA

Viruses possess small genomes made up of nucleic acid. Examples of viruses possessing genomes made up of DNA are known in the art and include, but are not limited to, poxvirus, herpes virus, adenovirus, papillomavirus, and parvovirus. Examples of viruses possessing genomes made up of RNA are likewise known in the art and include, but are not limited to, influenza virus, rotavirus, mumps virus, rabies virus, HIV/AIDS virus, corona virus, LCM virus and poliovirus. The viral genome can be either single- or double-stranded, and is packaged in a capsid, or protein coat, which in enveloped viruses is further enclosed by a lipid envelope. Nonenveloped viruses leave an infected cell by lysing and thereby killing the cell. Enveloped viruses can leave the cell by budding, without disrupting the plasma membrane and, therefore, without killing the cell. Enveloped viruses can thus cause chronic infections, in some cases helping transform an infected cell into a cancer cell. All viruses use the basic host cell machinery for most aspects of their reproduction, including transcription and translation. Many viruses encode proteins that modify the host transcription or translation apparatus to favor the synthesis of viral proteins over those of the host cell. The synthetic capability of the host cell is thus directed principally to the production of new virus particles. While most of the viral genome encodes mRNA that is translated into functional protein, small genomic regions of most viruses encode untranslated, or non-coding, RNAs.

miRNAs may provide several advantages for viruses seeking to reshape the cellular environment to maximize viral replication. miRNAs provide a highly specific way of downregulating the expression of host cell gene products that otherwise might interfere with some aspect of the viral replication cycle. In addition, the small size of mature miRNAs is advantageous given the tight size constraints of the viral genome, and miRNAs, unlike viral proteins, are not antigenic. miRNA expression is conceivably particularly beneficial for nuclear DNA viruses that establish long-term infections, as pri-miRNA processing takes place in the nucleus. Viruses known to encode miRNAs include members of the Herpesviridae (hCMV, KSHV, EBV, MHV68, rLCV) and Polyomaviridae (SV-40) families. In addition, the instant inventors discovered Virus-Associated (VA) RNAs that are transcribed from the Adenovirus genome and processed by Dicer to generate fragments of 21-23 nucleotides. These VA RNAs were shown to function as substrates and inhibitors of the RNAi pathway. Numerous other viruses also encode untranslated RNA sequences containing a high degree of secondary structure which, in many instances, bears structural similarity to miRNA precursors processed by Dicer. It therefore appears highly likely that virally encoded miRNAs are important to the virus life cycle in vivo.

### IV. VIRAL MODULATION OF CELLULAR miRNA EXPRESSION

Considering the prevalence of endogenous miRNA expression in eukaryotic cells, it is also likely that viral genomes or RNA transcripts are targeted by cellular miRNAs. A recent study reported that human miRNA miR-32 interacts with RNA of primate foamy virus type I (PFV-1) (Lecellier (2005) Science 308:557-560). Sequestration of miR-32 with antisense oligonucleotides resulted in an increase in viral replication, indicating that miR-32 impairs PFV-1 gene expression. It has further been suggested that the interaction of HIV with host T cells may be modulated by miRNAs, as at least five miRNAs expressed in human T cells have highly conserved predicted target sites within the nef and upr transcripts of HIV (Hariharan (2005) Biochem. Biophys. Res. Commun. 337:1214-1218). If cellular miRNAs are involved in inhibition of viral replication, it is possible that viruses counter this process by interfering with cellular miRNA expression. Indeed, PFV-1 has been shown to encode the protein Tas, which broadly suppresses miRNA activity. Remarkably, instances of positive regulation of cellular miRNA expression by viruses have also been reported. Human miRNA miR-122 interacts with the 5′-non-coding region of HCV and increases viral RNA production, through a mechanism which remains to be elucidated (Jopling (2005) Science 309:1577-1581). In addition, latency type III Epstein-Barr Virus (EBV) infections have been associated with induction of miR-155 in human B cells (Nair (2006) TRENDS in Microbiology 14:169-175), indicating that this miRNA is beneficial for EBV replication.

### V. VIRUS-SPECIFIC miRNA SIGNATURES

The present invention is based, at least in part, on the discovery that infection of host cells with a specific virus results in a distinct pattern of cellular miRNA expression that is unique to an individual virus species. The specific modulation of cellular miRNA expression that occurs following infection forms a unique “miRNA signature” characteristic of infection with a particular virus.

The present invention is further based on the discovery of distinct cellular miRNA signatures that are characteristic of the replication stage of a virus in a cell, e.g., virus-specific, replication stage-specific signatures. These signatures uniquely identify the replication stage of a particular virus in a cell, e.g., productive, persistent, latent, etc. Distinct miRNA signatures are also characteristic of the disease and pathological status of an individual infected with a virus.

Virus-induced modulation of cellular miRNA expression can occur through a number of different mechanisms, including but not limited to the following. Perturbations in genomic structure or chromosomal architecture can occur following viral integration into the host cell genome. Integration can cause deletions, amplifications or rearrangement of the surrounding DNA, which can affect the structure and/or expression of any associated miRNA genes. Structural RNAs, including miRNAs, encoded by the viral genome may interfere with RNAi pathways of the host cell by, for example, sequestering proteins necessary for RNAi such as those that make up RISC, or by targeting miRNA precursors, or mRNAs encoding proteins that mediate RNAi, for degradation. Alternatively, transcription factors encoded by the viral genome may be responsible for increasing production of miRNAs that are advantageous for viral replication.

The infection-associated miRNA signatures provide several levels of information. First, global patterns of miRNA expression during and after infection offer tools for the assessment of patient health and therapeutic intervention. Second, miRNA signatures will reveal biological pathways altered during infection that may be indicative of complications or additional therapeutic targets in an individual.

Accordingly, the present invention provides methods for identifying miRNA signatures associated with a particular virus (e.g., virus-specific miRNA signatures), with a specific replication stage of a virus (e.g., virus-specific, replication stage-specific miRNA signatures), or with the disease and pathological status of an individual infected with a virus. These methods include the steps of (a) reverse transcribing RNA from a test sample to provide a set of target oligonucleotides; (b) hybridizing the target oligodeoxynucleotides to a microarray comprising miRNA-specific probe oligonucleotides to provide a hybridization signal profile for the test sample; and (c) comparing the test sample hybridization signal profile to a hybridization signal profile generated from a control sample; wherein a set of alterations between the hybridization signals of the samples is identified as the miRNA signature.

The test sample selected for use in the methods of the invention will vary depending on the miRNA signature to be determined. A test sample may be obtained from, for example, a cell infected with a virus (e.g., Adenovirus, Cytomegalovirus (e.g., HCMV), Epstein Barr virus (EBV), Human Papilloma virus (HPV), MHV-68, Human Immunodeficiency Virus (HIV), Hepatitis A Virus (HAV), Hepatitis B Virus (HBV), Hepatitis C Virus (HCV), Hepatitis E Virus (HEV), Rubella Virus, Mumps Virus, Measles Virus, Respiratory Syncytial Virus, Human T-cell Leukemia Virus, Lentivirus, Herpes Simplex Virus (e.g., Herpes Simplex 1 (HSV1), Herpes Simplex 2 (HSV2)), Varicella-Zoster Virus, Human Herpesviruses 6A, 6B, and 7, Kaposi's Sarcoma-Associated Herpesvirus (e.g., KSHV, HHV8), Cercopithecine Herpesvirus, Hepatitis Delta Virus, Dengue Virus, Foot and Mouth Disease Virus, Polyomavirus (e.g., JC, BK), Poliovirus, Coxsackievirus, Echovirus, Rhinovirus, Vacciniavirus, Small Pox Virus, Influenza Virus, Avian Influenza Virus, etc.), a cell infected with a virus, wherein the virus is in a particular replication stage (e.g., productive infection, persistent infection, latent infection, etc.), or a subject infected with a virus, wherein the subject has a particular disease or pathological status. These examples are not intended to be limiting, as other test samples suitable for use in practicing the methods of the invention are readily apparent to one skilled in the art.

The control sample selected for use in the methods of the invention is any control or standard familiar to one of ordinary skill in the art that is useful for comparison purposes. A suitable control sample may be obtained from a cell or organism, e.g., a control or normal cell or organism, exhibiting, for example, normal traits. Such a control cell or organism is one which, for example, is not infected with a virus (e.g., an uninfected cell or organism, a mock-infected cell or organism). In one embodiment, a control sample has a value, level, feature, characteristic, property, etc. of a sample obtained from a normal cell or organism. For example, a cell or organism from which a control sample is derived may have a transcription rate, mRNA level, miRNA level, translation rate, protein level, biological activity, cellular characteristic or property, genotype, phenotype, etc. that is typical of a normal cell or organism. In one embodiment, a feature of a control sample, for example, a value, level, characteristic, property, etc., has been predefined (e.g., a level of expression of an miRNA, a hybridization signal profile, etc.). In this embodiment, the predefined feature is used for comparison purposes with the test sample. These examples are not intended to be limiting, as other test samples suitable for use in practicing the methods of the invention are readily apparent to one skilled in the art.

A. Determination of miRNA Expression Levels

The methods of the invention require determining miRNA expression levels in a cell or in a biological sample. Methods for determining miRNA expression levels in cells or biological samples are within the level of skill in the art. Such methods include, but are not limited to, northern blot analysis, in situ hybridization, and quantitative reverse transcriptase polymerase chain reaction. In a preferred embodiment, miRNA expression levels are determined by microarray analysis.

Total cellular RNA can be purified from cells by homogenization in the presence of nucleic acid extraction buffer, followed by centrifugation. Nucleic acids are precipitated, and DNA is removed by treatment with DNase and precipitation.

RNA molecules can be separated by gel electrophoresis on agarose gels according to standard techniques, and transferred to nitrocellulose filters by, e.g., the so-called “Northern Blot” technique. The RNA is then immobilized on the filters by heating. Detection and quantification of specific RNA is accomplished using appropriately labeled DNA or RNA probes complementary to the RNA in question (see, for example, Molecular Cloning: A Laboratory Manual, J. Sambrook et al., eds., 2^{nd} Edition, Cold Spring Harbor Laboratory Press, 1989, Chapters 10 and 11, the disclosures of which are incorporated herein by reference.

Suitable probes for Northern blot hybridization of a given miRNA gene product can be produced using the nucleotide sequence of an miRNA. miRNA, hairpin pre-miRNA and miRNA* sequences known in the art are listed by name and accession number in Tables 1-3. In a preferred embodiment, probes are produced using the nucleic acid sequences of human, murine, avian, or viral origin corresponding to the miRNAs, hairpin pre-miRNAs and miRNAs* described in Tables 1-3. The nucleic acid sequences corresponding to the miRNAs, hairpin pre-miRNAs and miRNA*s described in Tables 1-3 are available from the “miRBase::Sequences” database of the Wellcome Trust Sanger Institute (http://microrna.sangetac.uk/sequences/index.shtml). miRNA, hairpin miRNA, and miRNA* sequence information can be found in Release 10.0 (Aug. 2, 2007), Release 10.1 (Dec. 19, 2007), Release 11.0 (Sep. 1, 2008) and Release 12.0 (Sep. 4, 2008), available for download from the miRBase::Sequences database of the Wellcome Trust Sanger Institute (http://microrna.sangetac.uk/sequences/ftp.shtml, previous releases). This database is described further in the following papers, incorporated by reference herein in their entirety: Griffiths-Jones S, Saini H K, van Dongen S, Enright A J., miRBase: tools for microRNA genomics, NAR 2008 36(Database Issue):D154-D158; Griffiths-Jones S, Grocock R J, van Dongen S, Bateman A, Enright A J., miRBase: microRNA sequences, targets and gene nomenclature, NAR 2006 34(Database Issue):D140-D144; and Griffiths-Jones S., The microRNA Registry, NAR 2004 32(Database Issue):D109-D111. Nomenclature conventions used to describe miRNA sequences are described further in Ambros V, Bartel B, Bartel D P, Burge C B, Carrington J C, Chen X, Dreyfuss G, Eddy S R, Griffiths-Jones S, Marshall M, Matzke M, Ruvkun G, Tuschl T., A uniform system for microRNA annotation, RNA 2003 9(3):277-279, the entire contents of which are incorporated herein by reference.

Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are herein incorporated by reference.

For example, the nucleic acid probe can be labeled with, e.g., a radionuclide such as ³H, ³²P, ³³P, ¹⁴C, or ³⁵S; a heavy metal; or a ligand capable of functioning as a specific binding pair member for a labeled ligand (e.g., biotin, avidin or an antibody), a fluorescent molecule, a chemiluminescent molecule, an enzyme or the like.

Probes can be labeled to high specific activity by either the nick translation method of Rigby et al. (Rigby (1977), J. Mol. Biol. 113:237-251), or by the random priming method of Fienberg et al. (Fienberg (1983), Anal. Biochem. 132:6-13, the entire disclosures of which are herein incorporated by reference. The latter is the method of choice for synthesizing ³²P-labeled probes of high specific activity from single-stranded DNA or from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of miRNA gene transcript levels. Using another approach, miRNA gene transcript levels can be quantified by computerized imaging systems, such the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N-(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to Northern and other RNA blotting hybridization techniques, determining the levels of RNA transcripts can be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference. Suitable probes for in situ hybridization of a given miRNA gene product can be produced using the nucleotide sequence of an miRNA. miRNA, miRNA* and pre-miRNA hairpin sequences known in the art correspond to the miRNAs, miRNA*s and pre-miRNA hairpins described in Tables 1-3. In a preferred embodiment, probes are produced using the nucleic acid sequences corresponding to human, murine, avian, or viral origin provided in Tables 1-3.

The relative number of miRNA gene transcripts in cells can also be determined by reverse transcription of miRNA gene transcripts, followed by amplification of the reverse-transcribed transcripts by polymerase chain reaction (RT-PCR). The levels of miRNA gene transcripts can be quantified in comparison with an internal standard, for example, the level of mRNA from a “housekeeping” gene present in the same sample. A suitable “housekeeping” gene for use as an internal standard includes, e.g., myosin or glyceraldehyde-3-phosphate dehydrogenase (GAPDH). The methods for quantitative RT-PCR and variations thereof are within the skill in the art.

In some embodiments, it is desirable to simultaneously determine the expression level of a plurality of different of miRNAs in a sample. In certain instances, it may be desirable to determine the expression level of the transcripts of all known miRNAs associated with a specific miRNA signature. Assessing expression levels for hundreds of miRNAs is time consuming and requires a large amount of total RNA (at least 20 μg for each Northern blot) and autoradiographic techniques that require radioactive isotopes. To overcome these limitations, an oligolibrary in microchip format may be constructed containing a set of probe oligonucleotides specific for a set of miRNA genes. In one embodiment, the oligolibrary contains probes corresponding to all known miRNAs from the human genome. In alternate embodiments, the oligolibrary contains probes corresponding to all known miRNAs from the avian or murine genomes.

The set of mature miRNAs, miRNA*s, and pre-miRNA hairpin precursors known in the art at the time of filing of the instant application may be found in Tables 1-3. The nucleic acid sequences corresponding to the miRNAs, hairpin pre-miRNAs and miRNA*s described in Tables 1-3 are available from the “miRBase::Sequences” database of the Wellcome Trust Sanger Institute (http://microrna.sangetac.uk/sequences/index.shtml). miRNA, hairpin miRNA, and miRNA* sequence information can be found in miRBase Sequence Download Release 10.0 (Aug. 2, 2007), Release 10.1 (Dec. 19, 2007), Release 11.0 (Sep. 1, 2008) and Release 12.0 (Sep. 4, 2008), available for download from the miRBase::Sequences database of the Wellcome Trust Sanger Institute (http://microrna.sangetac.uk/sequences/ftp.shtml, previous releases). This database is described further in the following papers, incorporated by reference herein in their entirety: Griffiths-Jones S, Saini H K, van Dongen S, Enright A J., miRBase: tools for microRNA genomics, NAR 2008 36(Database Issue):D154-D158; Griffiths-Jones S, Grocock R J, van Dongen S, Bateman A, Enright A J., miRBase: microRNA sequences, targets and gene nomenclature, NAR 2006 34(Database Issue):D140-D144; and Griffiths-Jones S., The microRNA Registry, NAR 2004 32(Database Issue):D109-D111. Nomenclature conventions used to describe miRNA sequences are described further in Ambros V, Bartel B, Bartel D P, Burge C B, Carrington J C, Chen X, Dreyfuss G, Eddy SR, Griffiths-Jones S, Marshall M, Matzke M, Ruvkun G, Tuschl T., A uniform system for microRNA annotation, RNA 2003 9(3):277-279, the entire contents of which are incorporated herein by reference.

The nucleic acid sequences corresponding to the miRNA, miRNA* and hairpin miRNAs described in Tables 1-3 are suitable for use in designing probes, oligonucleotides, primers, etc. for use in the methods and applications of the invention. This database is continually updated as new miRNAs are identified. As a skilled artisan would appreciate, newly identified miRNA sequences may also be used in the practice of the instant invention. In a preferred embodiment, miRNA sequences of human, murine, avian, or viral origin are used in practicing the methods and applications of the invention.

The microchip is prepared from gene-specific oligonucleotide probes generated from known miRNAs. According to one embodiment, the array contains two different oligonucleotide probes for each miRNA, one containing the active sequence and the other being specific for the precursor of the miRNA. The array may also contain controls such as one or more (e.g. mouse) sequences differing from (e.g. human) orthologs by only a few bases, which can serve as controls for hybridization stringency conditions. tRNAs from both species may also be printed on the microchip, providing an internal, relatively stable positive control for specific hybridization. One or more appropriate controls for non-specific hybridization may also be included on the microchip. For this purpose, sequences are selected based upon the absence of any homology with any known miRNAs.

The microchip may be fabricated by techniques known in the art. For example, probe oligonucleotides of an appropriate length, e.g., 40 nucleotides, are 5′-amine modified at position C6 and printed using commercially available microarray systems, e.g., the GeneMachine OmniGrid™ 100 Microarrayer and Amersham CodeLink™ activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g. 6×SSPE/30% formamide at 25° C. for 18 hours, followed by washing in 0.75×TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary miRNAs, in the sample. According to one embodiment, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. The intensity of each spot on the array is proportional to the abundance of the corresponding miRNA in the sample.

The use of the array has several advantages for miRNA expression detection. First, the global expression of several hundred genes can be identified in a single sample at one time point. Second, through careful design of the oligonucleotide probes, expression of both mature and precursor molecules can be identified. Third, in comparison with Northern blot analysis, the chip requires a small amount of RNA, and provides reproducible results using 2.5 μg of total RNA. The relatively limited number of miRNAs (a few hundred per species) potentially allows the construction of a common microarray for several species, with distinct oligonucleotide probes for each. Such a tool would allow for analysis of trans-species expression for each known miRNA under various conditions. The microanay methods described herein are useful for both the identification of miRNA signatures and the diagnostic and therapeutic applications of the invention.

In the screening assays of the invention, cells or biological samples as described above can further comprise suitable controls. Such suitable controls will be obvious to one skilled in the art and are considered part of the common knowledge. The relative miRNA expression in the control or normal samples can further be determined with respect to one or more RNA expression standards. The standards can comprise, for example, a zero miRNA gene expression level, the miRNA gene expression level in a standard cell line, or the average level of miRNA gene expression previously obtained for a population of normal human controls.

### VI. THERAPEUTIC AND DIAGNOSTIC APPLICATIONS

As described herein, miRNA signatures identified using the methods of the present invention have therapeutic and diagnostic utility. miRNA signatures can further be used experimentally, for example, in identifying host cell factors that are important for viral pathogenesis.

### A. Screening Assays

1. Identification of Druggable Targets

Analysis of virus-specific modifications to miRNA expression levels will provide information about the cellular factors that are important/essential for virus infection or the establishment of productive, chronic or latently infected states. These miRNAs and the genes they regulate are targets for therapeutic treatment of the virus infection. Accordingly, the present invention provides methods for identification of druggable targets, involving (a) obtaining a cell or organism capable of executing RNAi; (b) infecting said cell or organism with a virus; and (c) assaying for expression of an miRNA; wherein a change in expression of an miRNA indicates that the miRNA, or a gene targeted by the miRNA, is a druggable target. A change in expression of an miRNA is measured relative to, for example, a suitable control, e.g., the miRNA expression level of a cell prior to infection with the virus, the miRNA expression level of a cell following mock infection, or a predefined miRNA expression level associated with an uninfected cell. Additional suitable controls will be obvious to one skilled in the art, and are further described herein. In another embodiment, a protein encoded by an RNA targeted by an miRNA, wherein the miRNA undergoes a change in expression following infection with a virus, is a druggable target. In various embodiments, the targeted mRNA encodes a viral protein or a cellular protein.

For example, the level of at least one miRNA gene product produced from an miRNA gene can be measured in a cell, or in a biological sample obtained from an organism infected with a virus. An alteration (i.e., an upregulation or a downregulation) in the level of miRNA gene product in the cell or sample is indicative that the miRNA gene product, an mRNA targeted by the miRNA, or a protein encoded by such an mRNA, is important or essential for some aspect of virus infection and, as such, makes a suitable druggable target. Accordingly, the druggable targets are preferably anti-viral druggable targets. In cases where the miRNA is known or suspected to play a role in a particular function, e.g., a cellular or viral function, a subset of candidate mRNAs, e.g., cellular or viral RNAs, previously identified as being involved in that function can be selected and analyzed for changes in gene expression. A change in expression of such mRNAs indicates that these mRNAs may additionally be identified as druggable targets. Also provided by the present invention are druggable targets (e.g., antiviral drug targets) identified using the methods described herein.

2. Identification of Antiviral Agents

In a related aspect, the present invention provides methods of identifying antiviral agents, involving (a) contacting a cell with a test agent, said cell comprising an RNAi pathway and a virus, wherein said virus modulates the expression of one or more cellular miRNAs; (b) assaying for a change in expression of said miRNAs; and (c) identifying a test agent based on its ability to inhibit modulation of cellular miRNA expression by the virus. As described herein, a cellular miRNA whose expression is modulated by a virus is likely important or essential for infection and replication of that virus in a cell. A decrease in expression of an miRNA following virus infection is an indication that the miRNA is deleterious or inhibitory to some aspect of the viral life cycle. Likewise, an increase in expression of an miRNA following virus infection is an indication that the miRNA is advantageous to some aspect of the viral life cycle. Restoration to endogenous levels of expression of an miRNA displaying an altered pattern of expression following viral infection may delay or inhibit viral infection or replication. Likewise, restoration to endogenous levels of an mRNA, or a protein encoded by an mRNA, that is targeted by an miRNA displaying an altered pattern of expression following viral infection may delay or inhibit viral infection or replication. Accordingly, an agent capable of restoring endogenous levels of an miRNA, an mRNA, or a protein, as described above, is identified according to the methods of the present invention as an antiviral agent.

For example, in one embodiment, a virus increases the expression of one or more cellular miRNAs. In this embodiment, a test agent is identified as an antiviral agent based on its ability to inhibit the increase in expression of one or more cellular miRNAs caused by the virus. In another embodiment, a virus decreases the expression of one or more cellular miRNAs. In this embodiment, a test agent is identified as an antiviral agent based on its ability to inhibit the decrease in expression of one or more cellular miRNAs caused by the virus. In addition, a test agent that inhibits the alteration of an mRNA (or encoded protein) that is a target of an miRNA upregulated or down-regulated by a virus is further identified as an antiviral agent.

The test agents of the present invention can be obtained using any of the numerous approaches in combinatorial library methods known in the art, including biological libraries, spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). The test agents of the present invention can be obtained using nucleic acid libraries, e.g., complementary DNA libraries (see S. Y. Sing (2003) Methods Mol Biol 221:1-12), DNA or RNA aptamer libraries (see C. K. O'Sullivan 2002 Anal Bioanal Chem 372(1):44-48; J. J. Toulme 2000 Curr Opin Mol Ther 2(3):318-24; J. J. Toulme et al., 2001 Prog Nucleic Acid Res Mol Biol 69:1-46) and by using in vitro evolution approaches, e.g., in vitro evolution of nucleic acids (see, e.g., J. A. Bittker et al. 2002 Curr Opin Chem Biol 6(3):367-374).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

In a preferred embodiment, the library is a natural product library, e.g., a library produced by a bacterial, fungal, or yeast culture. In another preferred embodiment, the library is a synthetic compound library.

In an exemplary embodiment, the test agent is an siRNA, an antisense RNA, an shRNA, an expression vector (e.g., a plasmid expression vector or a viral expression vector), or a recombinant or synthetic polypeptide.

Compounds or agents identified according to the methods of the invention can be used therapeutically or prophylactically either alone or in combination. Accordingly, the present invention provides compositions comprising an agent identified using the methods described herein, and a pharmaceutically acceptable carrier. The invention further provides methods of treating or attenuating a viral infection in an organism by administering compositions that include an antiviral agent or compound identified according to the methods described herein, or a pharmaceutical composition including the same.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

Preferred cells for use in the screening assays of the invention are eukaryotic cells, although screening in prokaryotic cells is contemplated. In one embodiment, the cell is a plant cell. In another embodiment, the cell is an insect cell. In a preferred embodiment, the cell is a mammalian cell (e.g., a human cell or a murine cell). In another preferred embodiment, the cell is an avian cell.

### B. Diagnostic Applications

1. Identification of a Virus

miRNA signatures unique to specific viruses can be used as diagnostic indicators of infection. Accordingly, in one aspect, the present invention provides methods of detecting a virus in a sample. The sample used in this aspect can be derived from any source, but is preferably a cell sample or a biological sample. In an exemplary embodiment, the cell sample or biological sample is obtained from a tissue or organ. According to this method, RNA is isolated from the sample according to the methods described herein. The level of expression of one or more miRNAs previously identified as belonging to a virus-specific miRNA signature is determined, and is compared to the level of expression present in a control sample. Alterations in the expression of one or more miRNAs previously identified as belonging to a virus-specific miRNA signature relative to a control sample indicates the presence of the virus in a sample. Detecting such alterations can therefore be used to identify an underlying viral agent present in a diseased sample (e.g., a sample obtained from a diseased tissue or organ). Likewise, the absence of such alterations can be used to determine the lack of a viral agent in a sample.

In one embodiment, a virus is identified in a tissue or organ prior to transplantation. Exemplary tissues or organs that may be used for transplantation include, but are not limited to, kidney, liver, heart, lung and skin. Recipients of tissue or organ transplants are frequently administered immunosuppressive therapy in order to prevent transplant rejection. Accordingly, transplant recipients are particularly susceptible to infection, including infection by a viral agent present in the transplanted tissue or organ. Identification of a viral agent present in a tissue or organ prior to transplantation can prevent infection of the recipient which can ultimately lead to graft failure. Alternatively, identification of a viral agent present in a tissue or organ prior to transplantation can indicate that the recipient should be monitored for development of a viral infection following transplantation.

The cell or biological sample may also be obtained from a subject. In this embodiment, alterations in the expression of one or more miRNAs previously identified as belonging to a virus-specific miRNA signature relative to a control sample indicates that the subject is infected with a virus. Accordingly, the present invention provides methods for diagnosing a viral infection in subject. In one embodiment, miRNA signatures are used to diagnose a viral infection in a subject prior to or following receipt of an organ transplant.

2. Identification of the Replication Stage of a Virus

miRNA signatures unique to a specific replication stage of a given virus likewise have utility as diagnostic indicators of infection. Accordingly, in one aspect, the present invention provides methods of detecting the replication stage of a virus in a sample. The sample used in this aspect can be derived from any source, but is preferably a cell sample or a biological sample. In an exemplary embodiment, the cell sample or biological sample is obtained from a tissue or organ. According to this method, RNA is isolated from the sample according to the methods described herein. The level of expression of one or more miRNAs previously identified as belonging to a virus-specific, replication stage-specific miRNA signature is determined, and is compared to the level of expression present in a control sample. Alterations in the expression of one or more miRNAs previously identified as belonging to a virus-specific, replication stage-specific miRNA signature relative to a control sample indicates the replication stage of the virus in a sample. Detection of such alterations can therefore be used to define the replication stage of a viral agent present in a diseased sample (e.g., a sample obtained from a tissue or organ infected with the viral agent). In one embodiment, the tissue or organ is a tissue or organ transplant.

In one embodiment, the cell or biological sample is obtained from a subject. In this embodiment, alterations in the expression of one or more miRNAs previously identified as belonging to a virus-specific, replication stage-specific miRNA signature relative to a control sample indicates the replication stage of a virus that has infected the subject. Identification of the replication stage of a virus that has infected a subject is further useful as an indicator of disease progression. Accordingly, the present invention provides methods for diagnosing the progression of a viral infection in subject. In one embodiment, the subject is a tissue or organ transplant recipient.

3. Identification of the Disease or Pathological Status of a Subject Infected with a Virus

miRNA signatures identified as being indicative of a given disease or pathological state of an individual infected with a virus further have utility as diagnostic indicators of infection. Accordingly, in one aspect, the present invention provides methods of detecting the disease or pathological status of a subject infected with a virus. The sample used in this aspect is obtained from a subject infected with a virus. According to this method, RNA is isolated from the sample according to the methods described herein. The level of expression of one or more miRNAs previously identified as belonging to an miRNA signature associated with a specific disease or pathological state is determined, and is compared to the level of expression present in a control sample. Alterations in the expression of one or more miRNAs previously identified as belonging to an miRNA signature associated with a specific disease or pathological state, relative to a control sample, indicates the specific disease or pathological state of the subject infected with a virus. According to this method, the disease or pathological status of a subject infected with a virus is diagnosed. In one embodiment, the subject is a tissue or organ transplant recipient.

Taken together, the foregoing methods illustrate that miRNA signatures serve as biomarkers of viral infection, treatment and recovery. Once a unique miRNA signature characteristic of a particular virus is established, miRNA analysis from a diseased sample, tissue, or subject can be used to determine the presence or absence of the virus, its replication stage (e.g., productive, persistent, latent, etc.), and the disease and pathological status of an infected subject. This information can then be used to tailor a treatment strategy to the particular subject. Such a treatment strategy may include, for example, an antiviral agent identified according to the methods described herein. In one embodiment, the subject is a tissue or organ transplant recipient. Tissues or organs that may be used for transplantation include, but are not limited to, kidney, liver, heart, lung and skin. In an exemplary embodiment, the virus is cytomegalovirus.

The foregoing methods of detecting a virus in a sample, detecting the replication stage of a virus in a sample, diagnosing a viral infection in a subject, diagnosing the replication stage of a virus or the disease progression of a viral infection in a subject, and diagnosing the disease or pathological state of an individual infected with a virus require determining miRNA expression levels in a sample, preferably a cell or biological sample. Methods for determining miRNA expression levels are within the level of skill in the art. Such methods include northern blot analysis, in situ hybridization, and quantitative reverse transcriptase polymerase chain reaction. In a preferred embodiment, miRNA expression levels are determined by microarray analysis. Procedures for performing these methods are well-known in the art, and are further described herein.

The diagnostic applications of the invention require comparison with a suitable control sample. Cells or biological samples obtained from a normal cell, tissue, or subject (e.g., one which exhibits normal traits), as described above, can further comprise suitable controls. Such suitable controls will be obvious to one skilled in the art and are considered part of the common knowledge. The relative miRNA expression in the control or normal samples can further be determined with respect to one or more RNA expression standards. The standards can comprise, for example, a zero miRNA gene expression level, the miRNA gene expression level in a standard cell line, or the average level of miRNA gene expression obtained for a population of normal human controls. Alternatively, a feature of a control sample, for example, a value, level, characteristic, property, etc., has been predefined (e.g., a level of expression of an miRNA, a hybridization signal profile, etc.). In this embodiment, the miRNA expression levels present in a sample are compared with the pre-determined features of a control sample.

### VII. miRNA SIGNATURE-SPECIFIC MICROARRAYS, OLIGONUCLEOTIDE PRIMERS, AND KITS

Following the identification of an miRNA signature according to the methods of the present invention (e.g., a virus-specific miRNA signature, a virus-specific, replication stage-specific miRNA signature, a miRNA signature associated with a specific disease or pathological state of a subject infected with a virus, etc.), microarrays can be constructed which contain oligonucleotide probes that specifically recognize the miRNAs identified as comprising the miRNA signature. Such microarrays are useful in the diagnostic, therapeutic, and screening assays described herein. Microarrays containing probes recognizing the subset of miRNAs comprising an miRNA signature have the advantages of being cost-effective to produce and simpler to analyze, as they do not include extraneous probes that are not relevant to the detection of, i.e., a virus, the replication stage of a virus, or the disease or pathological state of a subject infected with a virus. Accordingly, the present invention provides, in one aspect, a microanay containing probes recognizing the miRNAs of an miRNA signature identified using the methods of the invention. In one embodiment, such a microarray also contains oligonucleotide probes specific for RNAs suitable for normalization purposes. Such probes are well known in the art, and include, for example, probes recognizing 18s ribosomal RNA, mRNA encoding GAPDH, mRNA encoding β-actin, etc.

In a related aspect, the present invention provides a set of one or more pairs of oligonucleotide primers designed to specifically amplify the miRNAs of an miRNA signature identified using the methods of the invention in a reverse-transcriptase polymerase chain reaction assay, preferably a real-time quantitative reverse-transcriptase polymerase chain reaction assay. Quantitative reverse-transcriptase polymerase chain reaction can provide a cost-effective alternative to microarray analysis when the number of miRNAs identified as part of an miRNA signature is relatively small.

The present invention further provides a kit containing a microanay that has miRNA-specific probe oligonucleotides which specifically recognize miRNAs that were identified as part of an miRNA signature using methods of the invention. The microarrays contained in the kit may also include probe oligonucleotides which specifically recognize RNAs suitable for normalization purposes. In certain embodiments the kit further contains a control sample. This sample can be derived from a control or normal cell, tissue, or subject, e.g., one that exhibits, for example, normal traits. In additional embodiments, the kits of the invention contain instructions for use. In an alternative aspect, the present invention provides a kit containing a set of one or more pairs of oligonucleotide primers designed to specifically amplify the miRNAs of an miRNA signature identified using the methods of the invention in a reverse-transcriptase polymerase chain reaction assay, preferably a real-time quantitative reverse-transcriptase polymerase chain reaction assay.

This invention is further illustrated by the following examples which should not be construed as limiting. The contents of all references, patents and published patent applications cited throughout this application are incorporated herein by reference.

### EXAMPLES

### Example I

### Identification of an Adenovirus (Wild-Type Ad-5) Specific miRNA Signature

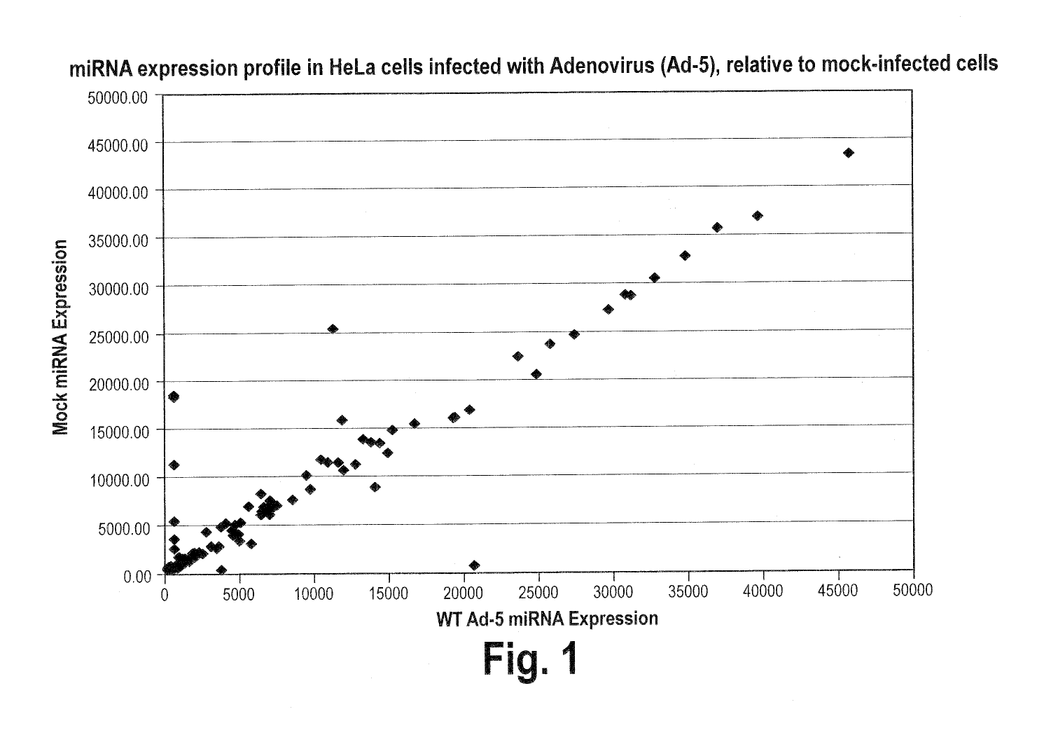

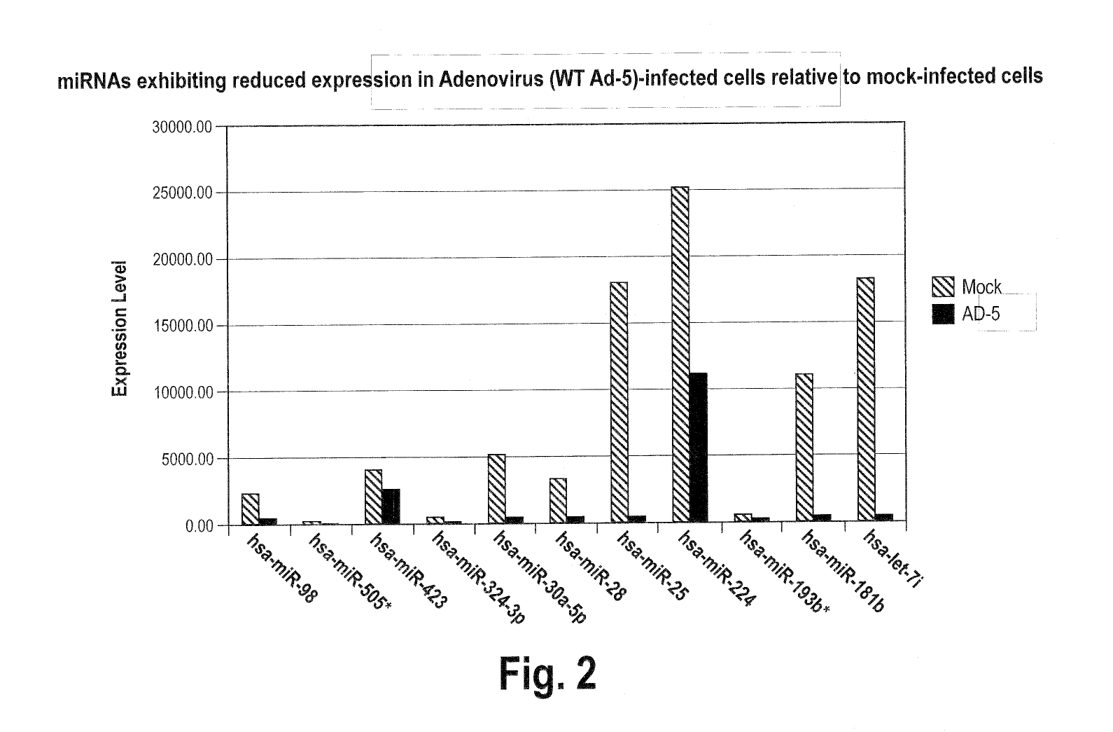

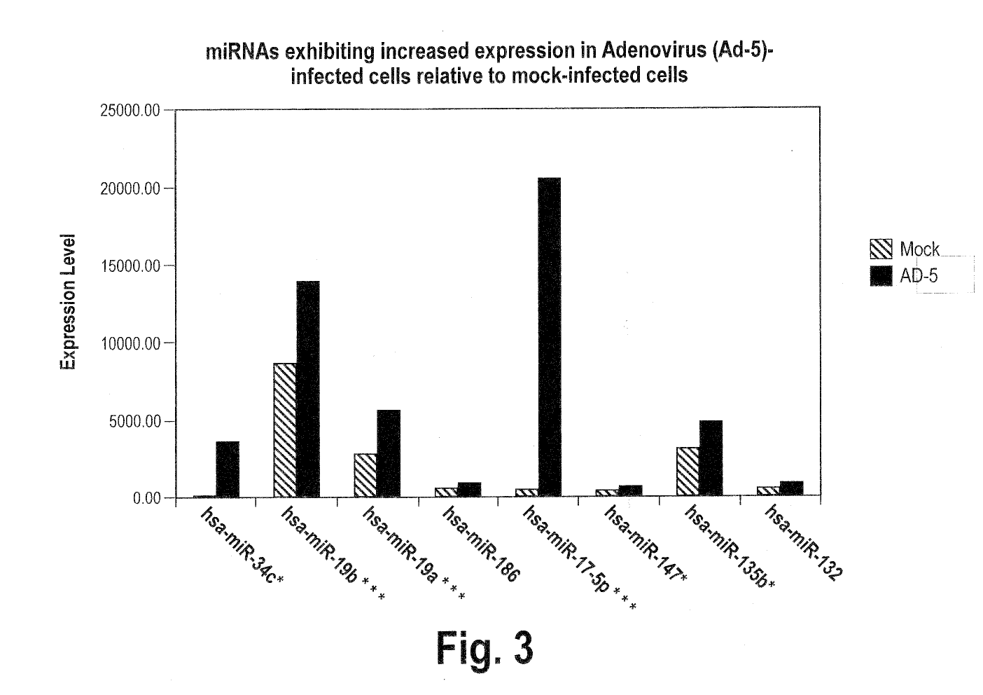

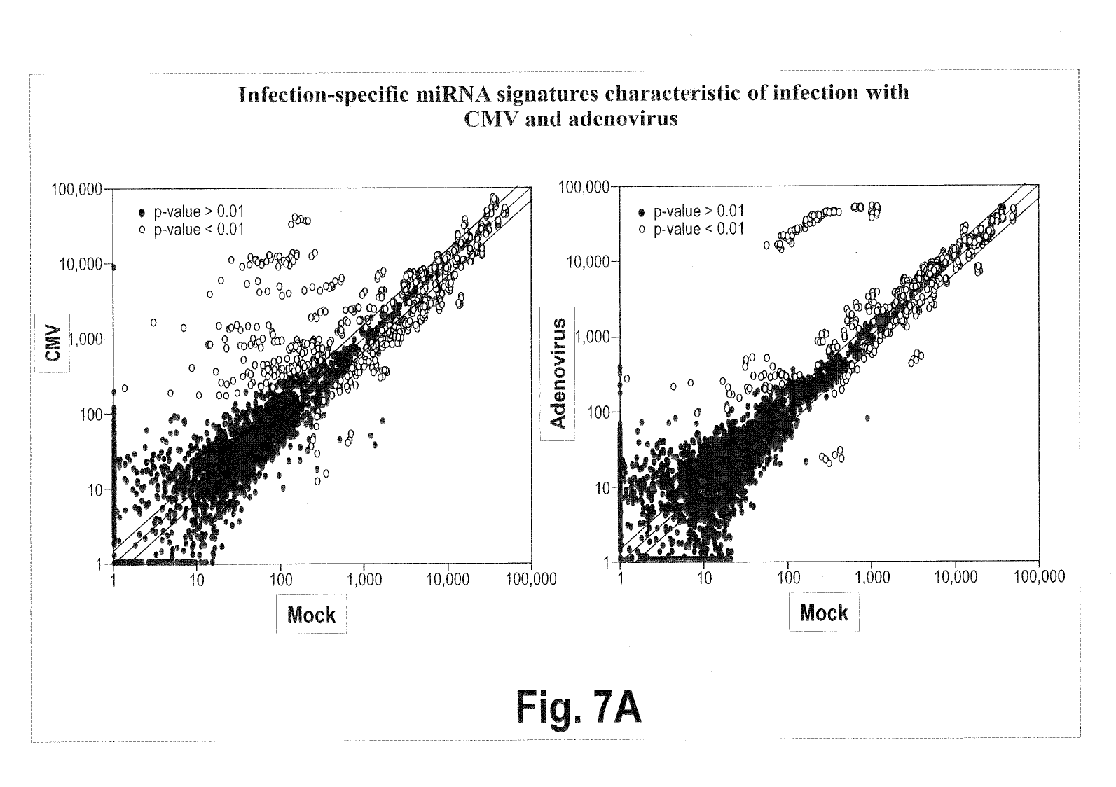

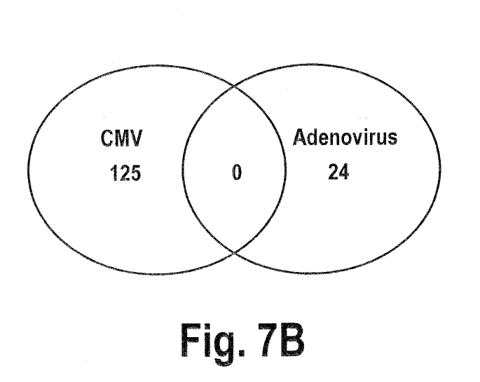

Global miRNA gene expression analysis was performed on RNA from mock or Adenovirus (WT Ad-5)-infected HeLa cells (MOI=5). RNA from adenovirus-infected cells was extracted using the miRvana Total RNA Isolation kit (Ambion, Inc., Austin, Tex.) at 24 hours post infection (hpi). Purification of the small RNA fraction and miRNA gene array was performed by LC Sciences (Houston, Tex.). All known human, adenoviral, and human cytomegalovirus miRNA and miRNA* sequences known at the time of the experiment were probed on microarray chips. These sequences are described in Appendices A-C of U.S. Provisional Application No. 60/967,780, filed Sep. 6, 2007. The entire contents of Appendices A-C of U.S. Provisional Application No. 60/967,780 are incorporated herein by reference. Data was normalized according to previously described methods (J. Lu et al., Nature (2005) 435:834-838). Widespread or global alterations in miRNA expression were not detected, indicating that differences between the test and control samples are specific to infection with Adenovirus. Differences in miRNA expression between mock-infected and Adenovirus-infected HeLa cells are shown in FIG. 1. The results indicate that a subset of miRNAs detected on the microarray are either upregulated or downregulated following Adenovirus infection. FIG. 2 depicts the expression level of miRNAs that are downregulated in Adenovirus-infected cells with respect to mock-infected control cells. FIG. 3 depicts the expression level of miRNAs that are upregulated in Adenovirus-infected cells with respect to mock-infected control cells. The subset of miRNAs upregulated or downregulated following adenovirus infection is unique and non-overlapping when compared with the subset of miRNAs upregulated or downregulated following infection with HCMV (FIG. 7). These results indicate that Adenovirus (Ad-5) infection is associated with a specific miRNA signature.

### Example II

### Identification of an HCMV (Ad-169) Specific miRNA Signature

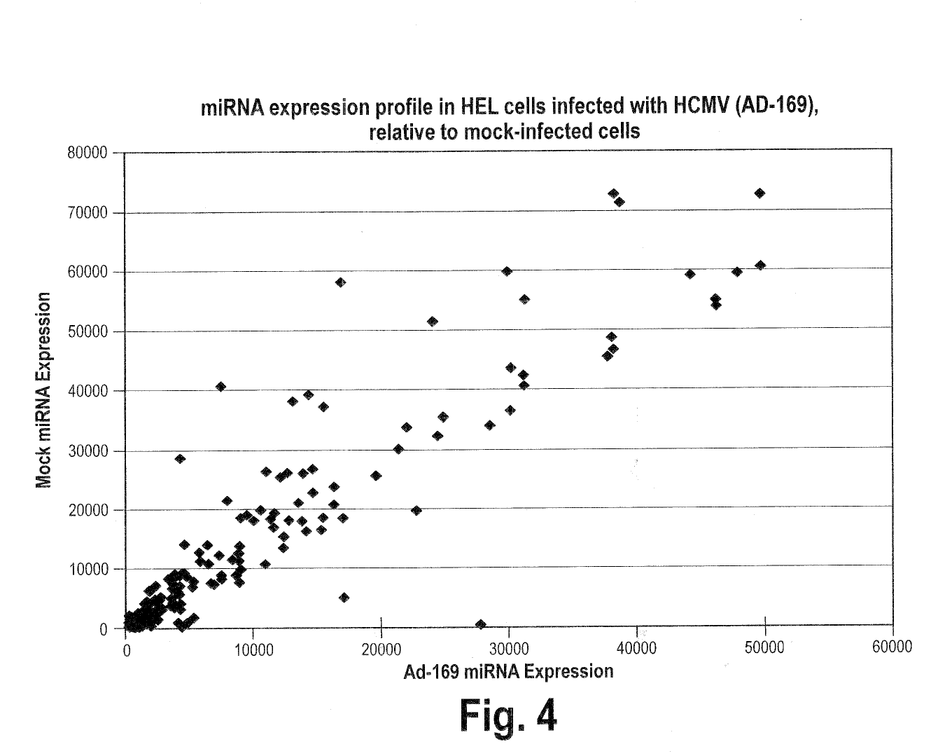

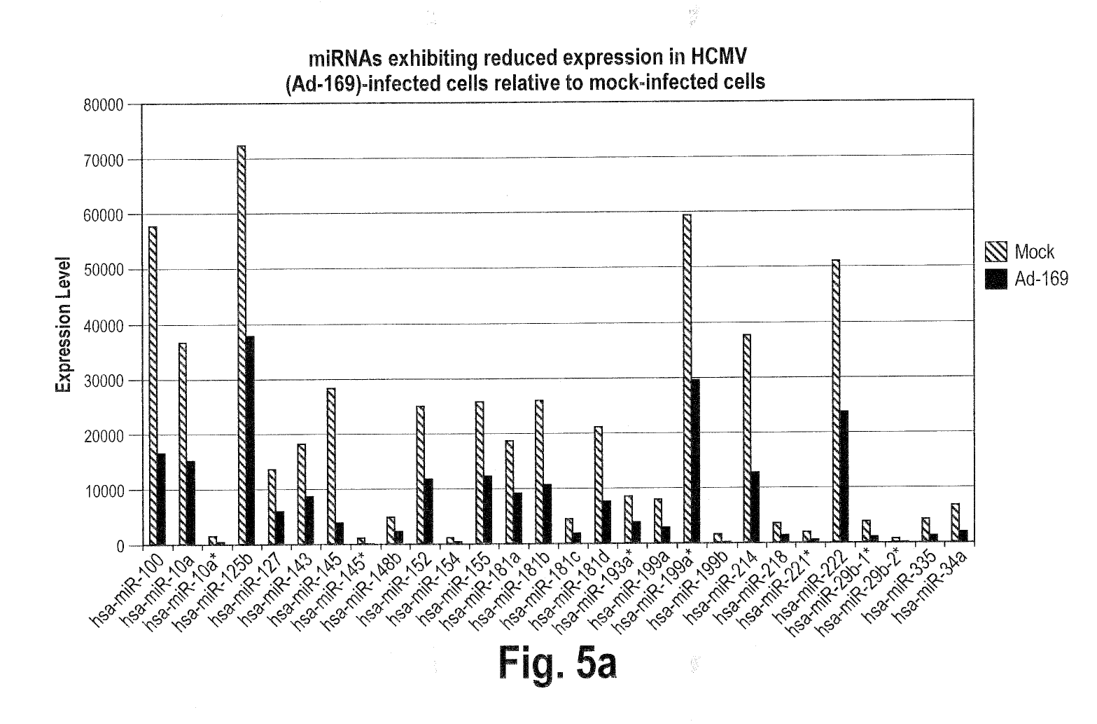

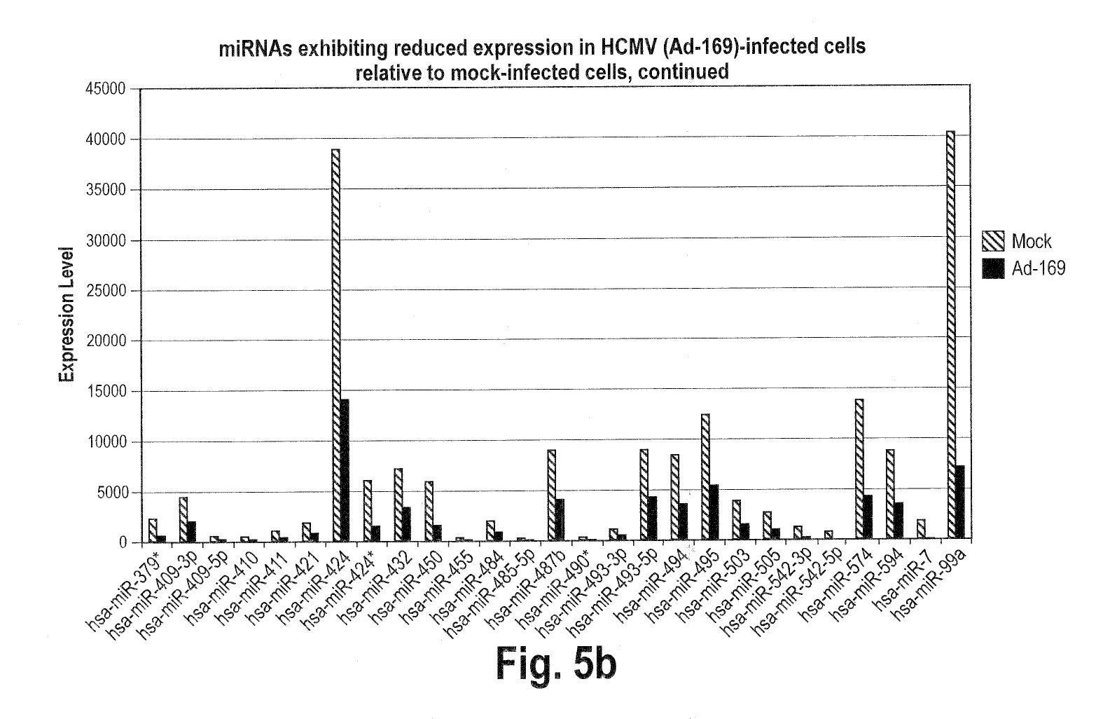

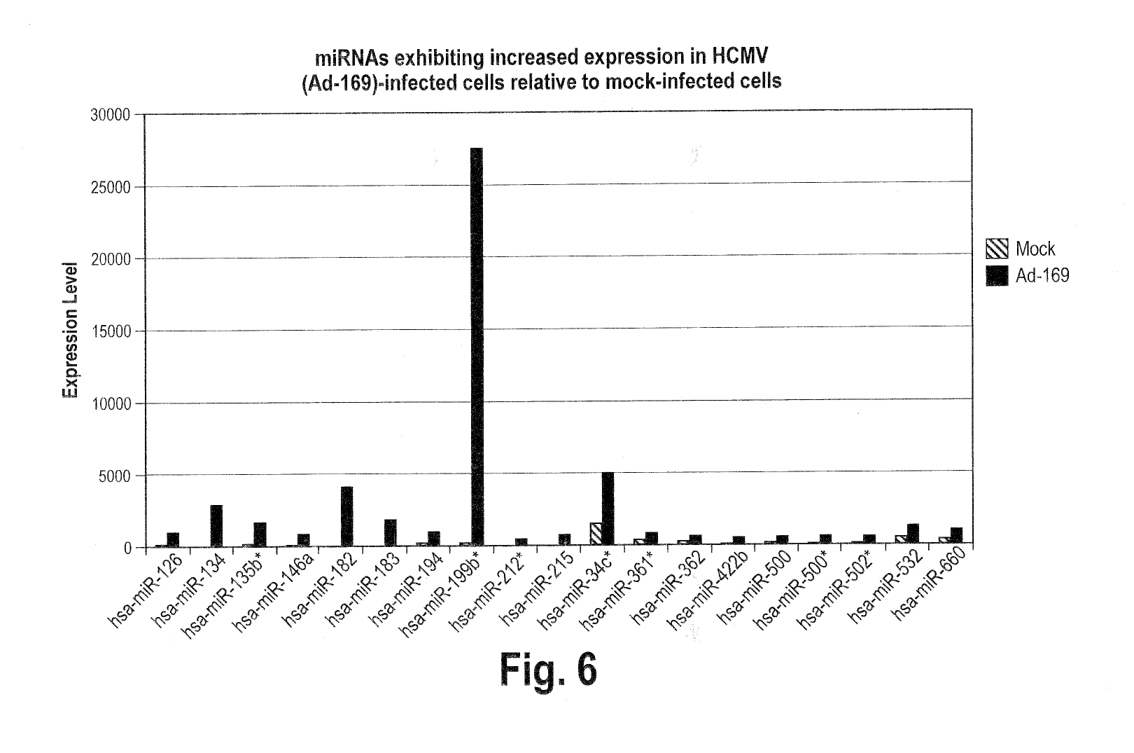

Global miRNA gene expression analysis was performed on RNA from mock or HCMV (Ad-169)-infected HEL cells (MOI=3). RNA from HCMV-infected cells was extracted using the miRvana Total RNA Isolation kit (Ambion, Inc., Austin, Tex.) at 48 hours post infection (hpi). Purification of the small RNA fraction and miRNA gene array was performed by LC Sciences (Houston, Tex.). All known human miRNA and miRNA* sequences known at the time of the experiment were probed on microarray chips. The foregoing sequences are also described in Appendices A-C of U.S. Provisional Application No. 60/967,780, filed Sep. 6, 2007, incorporated by reference herein in its entirety. Data was normalized according to previously described methods (J. Lu et al., Nature (2005) 435:834-838). Widespread or global alterations in miRNA expression were not detected, indicating that differences between the test and control samples are specific to infection with HCMV. Differences in miRNA expression between mock-infected and HCMV-infected HEL cells are shown in FIG. 4. The results indicate that a subset of miRNAs detected on the microarray are either upregulated or downregulated following HCMV infection. FIG. 5 depicts the expression level of miRNAs that are downregulated in HCMV-infected cells with respect to mock-infected control cells. FIG. 6 depicts the expression level of miRNAs that are upregulated in HCMV-infected cells with respect to mock-infected control cells. The subset of miRNAs upregulated or downregulated following HCMV infection is unique and non-overlapping when compared with the subset of miRNAs upregulated or downregulated following infection with adenovirus (FIG. 7). These results indicate that HCMV (Ad-169) infection is associated with a specific miRNA signature.

HCMV infection altered the expression of 25% of host miRNAs, whereas adenovirus infection only changed the expression of 5% of host miRNAs (see FIG. 7). These observations indicate that HCMV induces unique effects on miRNA expression. Given that each miRNA likely influences the expression of multiple genes, an implication of this radical effect on miRNA expression is that HCMV uses the host RNAi pathway to reprogram infected cells. Accordingly, miRNAs displaying altered expression following HCMV viral infection are suitable targets for HCMV therapy.

### Example III

### miRNA Signatures as Biomarkers for CMV Disease in Kidney Transplant Patients

Patients at highest risk of developing primary CMV infection after transplantation are CMV seronegative recipients (R−) of kidneys from CMV seropositive donors (D+). Baseline miRNA profiles are established in R− subjects following transplantation, but prior to presentation with CMV disease. Blood samples are collected from R− subjects for profiling. The monocytic cell population (the location of CMV during infection) is enriched from patient Peripheral Blood Mononuclear Cells (PBMCs). Small RNAs are extracted and processed for hybridization to miRNA microarrays using standard techniques. The miRNA expression profiles obtained from R− subjects prior to infection are compared across specimens to define “normal” miRNA expression patterns in R− kidney transplant recipients. The resulting miRNA profile serves as a baseline for asymptomatic transplant patients.

R− patients commonly experience primary CMV infection after transplantation, usually resulting from virus present in the allograft. Patients present with clinical symptoms of CMV infection (typically at least fever, malaise, and/or leucopenia), and/or CMV DNA is observed in the plasma. Patients are treated with high dose ganciclovir (GCV) until CMV DNA is undetectable for 2 to 3 consecutive weeks. Patients are then administered low dose GCV as secondary prophylaxis for 2 to 3 months. miRNA expression profiles are determined in blood specimens obtained from subjects at the time of presentation with CMV disease. In addition, miRNA expression profiles are determined in blood specimens obtained both upon completion of high dose and low dose prophylaxis treatment with GCV.

miRNA profiles are compared for each phase of CMV disease in individual patients. miRNA profiles are also compared across specimens at each phase of CMV disease to define miRNA expression signatures for CMV disease, therapeutic intervention, and recovery. In this way, miRNA signatures are correlated with observed disease status and predominant viral replication stage. These results demonstrate the relevance of infection-associated miRNA signatures as biomarkers during CMV disease and recovery in a clinically significant population.

### Example IV

### Detection of a Virus in a Sample

A blood sample is isolated from an R− subject following kidney transplantation from a CMV seropositive donor. The monocytic cell population is enriched from patient PBMCs. Small RNAs are extracted and processed for hybridization to miRNA microarrays using standard techniques. The miRNA expression profile of the sample is compared to the miRNA expression profile of asymptomatic transplant patients and to previously identified miRNA signatures characteristic of infection with CMV. The subset of miRNAs that are upregulated and downregulated in the miRNA expression profile of the sample is substantially similar to the miRNA signature characteristic of R-transplant patients with active CMV disease. Accordingly, the subject is identified as having a CMV infection.

TABLE 1
Mature miRNA Sequences

| | | | | | |
|---|---|---|---|---|---|
| >cel-let-7 | MIMAT0000001 | >mml-miR-183 | MIMAT0002783 | >oan-miR-155 | MIMAT0006988 |
| >cel-lin-4 | MIMAT0000002 | >mml-miR-96 | MIMAT0002784 | >oan-miR-1358 | MIMAT0006990 |
| >cel-miR-1 | MIMAT0000003 | >ptr-miR-183 | MIMAT0002785 | >oan-miR-26 | MIMAT0006991 |
| >cel-miR-2 | MIMAT0000004 | >ptr-miR-96 | MIMAT0002786 | >oan-miR-1359 | MIMAT0006993 |
| >cel-miR-34 | MIMAT0000005 | >ggo-miR-183 | MIMAT0002787 | >oan-miR-206 | MIMAT0006995 |
| >cel-miR-35 | MIMAT0000006 | >ggo-miR-96 | MIMAT0002788 | >oan-miR-133b | MIMAT0006997 |
| >cel-miR-36 | MIMAT0000007 | >sla-miR-183 | MIMAT0002789 | >oan-miR-190b | MIMAT0006998 |
| >cel-miR-37 | MIMAT0000008 | >sla-miR-96 | MIMAT0002790 | >oan-miR-194 | MIMAT0006999 |
| >cel-miR-38 | MIMAT0000009 | >mne-miR-183 | MIMAT0002791 | >oan-miR-215 | MIMAT0007000 |
| >cel-miR-39 | MIMAT0000010 | >mne-miR-96 | MIMAT0002792 | >oan-miR-1360 | MIMAT0007002 |
| >cel-miR-40 | MIMAT0000011 | >ppa-miR-183 | MIMAT0002793 | >oan-miR-147 | MIMAT0007003 |
| >cel-miR-41 | MIMAT0000012 | >ppa-miR-96 | MIMAT0002794 | >oan-miR-153 | MIMAT0007005 |
| >cel-miR-42 | MIMAT0000013 | >ggo-miR-106a | MIMAT0002795 | >oan-miR-128 | MIMAT0007006 |
| >cel-miR-43 | MIMAT0000014 | >age-miR-106a | MIMAT0002796 | >oan-miR-146b | MIMAT0007007 |
| >cel-miR-44 | MIMAT0000015 | >ppa-miR-106a | MIMAT0002797 | >oan-miR-458 | MIMAT0007010 |
| >cel-miR-45 | MIMAT0000016 | >mml-miR-106a | MIMAT0002798 | >oan-miR-101 | MIMAT0007011 |
| >cel-miR-46 | MIMAT0000017 | >ppy-miR-106a | MIMAT0002799 | >oan-miR-449c | MIMAT0007012 |
| >cel-miR-47 | MIMAT0000018 | >ptr-miR-106a | MIMAT0002800 | >oan-miR-449b | MIMAT0007013 |
| >cel-miR-48 | MIMAT0000019 | >sla-miR-106a | MIMAT0002801 | >oan-miR-449a | MIMAT0007015 |
| >cel-miR-49 | MIMAT0000020 | >mne-miR-106a | MIMAT0002802 | >oan-miR-192 | MIMAT0007016 |
| >cel-miR-50 | MIMAT0000021 | >mne-miR-92 | MIMAT0002803 | >oan-miR-223 | MIMAT0007020 |
| >cel-miR-51 | MIMAT0000022 | >hsa-miR-488 | MIMAT0004763 | >oan-miR-1361 | MIMAT0007022 |
| >cel-miR-52 | MIMAT0000023 | >hsa-miR-489 | MIMAT0002805 | >oan-miR-1362 | MIMAT0007023 |
| >cel-miR-53 | MIMAT0000024 | >hsa-miR-490-5p | MIMAT0004764 | >oan-miR-1363 | MIMAT0007025 |
| >cel-miR-54 | MIMAT0000025 | >hsa-miR-490-3p | MIMAT0002806 | >oan-miR-1364 | MIMAT0007027 |
| >cel-miR-55 | MIMAT0000026 | >hsa-miR-491-5p | MIMAT0002807 | >oan-miR-1365 | MIMAT0007028 |
| >cel-miR-56 | MIMAT0000028 | >hsa-miR-491-3p | MIMAT0004765 | >oan-miR-1366 | MIMAT0007030 |
| >cel-miR-57 | MIMAT0000029 | >hsa-miR-511 | MIMAT0002808 | >oan-miR-1367 | MIMAT0007032 |
| >cel-miR-58 | MIMAT0000030 | >hsa-miR-146b-5p | MIMAT0002809 | >oan-miR-1368 | MIMAT0007033 |
| >cel-miR-59 | MIMAT0000031 | >hsa-miR-146b-3p | MIMAT0004766 | >oan-miR-1369 | MIMAT0007034 |
| >cel-miR-60 | MIMAT0000032 | >hsa-miR-202 | MIMAT0002811 | >oan-miR-1370 | MIMAT0007035 |
| >cel-miR-61 | MIMAT0000033 | >hsa-miR-492 | MIMAT0002812 | >oan-miR-1371 | MIMAT0007036 |
| >cel-miR-62 | MIMAT0000034 | >hsa-miR-493 | MIMAT0003161 | >oan-miR-30f | MIMAT0007037 |
| >cel-miR-63 | MIMAT0000035 | >hsa-miR-432 | MIMAT0002814 | >oan-let-7g | MIMAT0007038 |
| >cel-miR-64 | MIMAT0000036 | >hsa-miR-494 | MIMAT0002816 | >oan-miR-1372 | MIMAT0007040 |
| >cel-miR-65 | MIMAT0000037 | >hsa-miR-495 | MIMAT0002817 | >oan-miR-33b-3p | MIMAT0009191 |
| >cel-miR-66 | MIMAT0000038 | >hsa-miR-496 | MIMAT0002818 | >oan-miR-30d | MIMAT0007041 |
| >cel-miR-67 | MIMAT0000039 | >hsa-miR-193b | MIMAT0002819 | >oan-miR-30b | MIMAT0007043 |
| >cel-miR-70 | MIMAT0000042 | >hsa-miR-497 | MIMAT0002820 | >oan-miR-184 | MIMAT0007045 |
| >cel-miR-71 | MIMAT0000043 | >hsa-miR-181d | MIMAT0002821 | >oan-miR-100 | MIMAT0007046 |
| >cel-miR-72 | MIMAT0000044 | >hsa-miR-512-5p | MIMAT0002822 | >oan-miR-125 | MIMAT0007048 |
| >cel-miR-73 | MIMAT0000045 | >hsa-miR-512-3p | MIMAT0002823 | >oan-miR-15b | MIMAT0007050 |
| >cel-miR-74 | MIMAT0000046 | >hsa-miR-498 | MIMAT0002824 | >oan-miR-16b | MIMAT0007052 |
| >cel-miR-75 | MIMAT0000047 | >hsa-miR-520e | MIMAT0002825 | >oan-miR-1373 | MIMAT0007054 |
| >cel-miR-76 | MIMAT0000048 | >hsa-miR-515-5p | MIMAT0002826 | >oan-miR-1374 | MIMAT0007055 |
| >cel-miR-77 | MIMAT0000049 | >hsa-miR-515-3p | MIMAT0002827 | >oan-miR-222b | MIMAT0007057 |
| >cel-miR-78 | MIMAT0000050 | >hsa-miR-519e | MIMAT0002829 | >oan-miR-181c | MIMAT0007058 |
| >cel-miR-79 | MIMAT0000051 | >hsa-miR-520f | MIMAT0002830 | >oan-miR-23a | MIMAT0007061 |
| >cel-miR-227 | MIMAT0000052 | >hsa-miR-519c-5p | MIMAT0002831 | >oan-miR-27a | MIMAT0007063 |
| >cel-miR-80 | MIMAT0000053 | >hsa-miR-519c-3p | MIMAT0002832 | >oan-miR-1375 | MIMAT0007064 |
| >cel-miR-81 | MIMAT0000054 | >hsa-miR-520a-5p | MIMAT0002833 | >oan-miR-1421k | MIMAT0007066 |
| >cel-miR-82 | MIMAT0000055 | >hsa-miR-520a-3p | MIMAT0002834 | >oan-miR-1421l | MIMAT0007068 |
| >cel-miR-83 | MIMAT0000056 | >hsa-miR-526b | MIMAT0002835 | >oan-miR-1419a | MIMAT0007071 |
| >cel-miR-84 | MIMAT0000057 | >hsa-miR-519b-5p | MIMAT0005454 | >oan-miR-1421m | MIMAT0007072 |
| >cel-miR-85 | MIMAT0000058 | >hsa-miR-519b-3p | MIMAT0002837 | >oan-miR-1421n | MIMAT0007074 |
| >cel-miR-86 | MIMAT0000059 | >hsa-miR-525-5p | MIMAT0002838 | >oan-miR-1421o | MIMAT0007076 |
| >cel-miR-87 | MIMAT0000060 | >hsa-miR-525-3p | MIMAT0002839 | >oan-miR-1421p | MIMAT0007078 |
| >cel-miR-90 | MIMAT0000061 | >hsa-miR-523 | MIMAT0002840 | >oan-miR-1421q | MIMAT0007080 |
| >hsa-let-7a | MIMAT0000062 | >hsa-miR-518f | MIMAT0002842 | >oan-miR-1421r | MIMAT0007082 |
| >hsa-let-7b | MIMAT0000063 | >hsa-miR-520b | MIMAT0002843 | >oan-miR-1421s | MIMAT0007084 |
| >hsa-let-7c | MIMAT0000064 | >hsa-miR-518b | MIMAT0002844 | >oan-miR-1421t | MIMAT0007086 |
| >hsa-let-7d | MIMAT0000065 | >hsa-miR-526a | MIMAT0002845 | >oan-miR-1421u | MIMAT0007088 |
| >hsa-let-7e | MIMAT0000066 | >hsa-miR-520c-5p | MIMAT0005455 | >oan-miR-150 | MIMAT0007090 |
| >hsa-let-7f | MIMAT0000067 | >hsa-miR-520c-3p | MIMAT0002846 | >oan-miR-1422j | MIMAT0007093 |
| >hsa-miR-15a | MIMAT0000068 | >hsa-miR-518c | MIMAT0002848 | >oan-miR-1422d | MIMAT0007094 |
| >hsa-miR-16 | MIMAT0000069 | >hsa-miR-524-5p | MIMAT0002849 | >oan-miR-590 | MIMAT0007097 |
| >hsa-miR-17 | MIMAT0000070 | >hsa-miR-524-3p | MIMAT0002850 | >oan-miR-130a | MIMAT0007098 |
| >hsa-miR-18a | MIMAT0000072 | >hsa-miR-517a | MIMAT0002852 | >oan-miR-1422m | MIMAT0007099 |
| >hsa-miR-19a | MIMAT0000073 | >hsa-miR-519d | MIMAT0002853 | >oan-miR-1422n | MIMAT0007102 |
| >hsa-miR-19b | MIMAT0000074 | >hsa-miR-521 | MIMAT0002854 | >oan-miR-1422o | MIMAT0007104 |
| >hsa-miR-20a | MIMAT0000075 | >hsa-miR-520d-5p | MIMAT0002855 | >oan-miR-1376 | MIMAT0007106 |
| >hsa-miR-21 | MIMAT0000076 | >hsa-miR-520d-3p | MIMAT0002856 | >oan-miR-1377 | MIMAT0007107 |
| >hsa-miR-22 | MIMAT0000077 | >hsa-miR-517b | MIMAT0002857 | >oan-miR-34 | MIMAT0007108 |
| >hsa-miR-23a | MIMAT0000078 | >hsa-miR-520g | MIMAT0002858 | >oan-miR-1378 | MIMAT0007110 |
| >hsa-miR-24 | MIMAT0000080 | >hsa-miR-516b | MIMAT0002859 | >oan-miR-31 | MIMAT0007111 |
| >hsa-miR-25 | MIMAT0000081 | >hsa-miR-518e | MIMAT0002861 | >oan-miR-124 | MIMAT0007114 |
| >hsa-miR-26a | MIMAT0000082 | >hsa-miR-518a-5p | MIMAT0005457 | >oan-miR-1379 | MIMAT0007115 |
| >hsa-miR-26b | MIMAT0000083 | >hsa-miR-518a-3p | MIMAT0002863 | >oan-miR-1380 | MIMAT0007117 |
| >hsa-miR-27a | MIMAT0000084 | >hsa-miR-518d-5p | MIMAT0005456 | >oan-miR-107 | MIMAT0007119 |
| >hsa-miR-28-5p | MIMAT0000085 | >hsa-miR-518d-3p | MIMAT0002864 | >oan-miR-1381 | MIMAT0007121 |
| >hsa-miR-28-3p | MIMAT0004502 | >hsa-miR-517c | MIMAT0002866 | >oan-miR-10a | MIMAT0007122 |
| >hsa-miR-29a | MIMAT0000086 | >hsa-miR-520h | MIMAT0002867 | >oan-miR-1382 | MIMAT0007124 |
| >hsa-miR-30a | MIMAT0000087 | >hsa-miR-522 | MIMAT0002868 | >oan-miR-126 | MIMAT0007126 |
| >hsa-miR-31 | MIMAT0000089 | >hsa-miR-519a | MIMAT0002869 | >oan-miR-429 | MIMAT0007128 |
| >hsa-miR-32 | MIMAT0000090 | >hsa-miR-527 | MIMAT0002862 | >oan-miR-551 | MIMAT0007130 |
| >hsa-miR-33a | MIMAT0000091 | >hsa-miR-516a-5p | MIMAT0004770 | >oan-miR-1383 | MIMAT0007131 |
| >hsa-miR-92a | MIMAT0000092 | >hsa-miR-516a-3p | MIMAT0006778 | >oan-miR-222a | MIMAT0007133 |
| >hsa-miR-93 | MIMAT0000093 | >hsa-miR-499-5p | MIMAT0002870 | >oan-miR-221 | MIMAT0007135 |
| >hsa-miR-95 | MIMAT0000094 | >hsa-miR-499-3p | MIMAT0004772 | >oan-miR-1384 | MIMAT0007136 |
| >hsa-miR-96 | MIMAT0000095 | >hsa-miR-500 | MIMAT0004773 | >oan-miR-148 | MIMAT0007138 |
| >hsa-miR-98 | MIMAT0000096 | >hsa-miR-501-5p | MIMAT0002872 | >oan-miR-196b | MIMAT0007139 |
| >hsa-miR-99a | MIMAT0000097 | >hsa-miR-501-3p | MIMAT0004774 | >oan-miR-145 | MIMAT0007141 |
| >hsa-miR-100 | MIMAT0000098 | >hsa-miR-502-5p | MIMAT0002873 | >oan-miR-143 | MIMAT0007144 |
| >hsa-miR-101 | MIMAT0000099 | >hsa-miR-502-3p | MIMAT0004775 | >oan-miR-20a | MIMAT0007145 |
| >hsa-miR-29b | MIMAT0000100 | >hsa-miR-503 | MIMAT0002874 | >oan-miR-19a | MIMAT0007148 |
| >hsa-miR-103 | MIMAT0000101 | >hsa-miR-504 | MIMAT0002875 | >oan-miR-1b | MIMAT0007149 |
| >hsa-miR-105 | MIMAT0000102 | >hsa-miR-505 | MIMAT0002876 | >oan-miR-133c | MIMAT0007150 |
| >hsa-miR-106a | MIMAT0000103 | >hsa-miR-513a-5p | MIMAT0002877 | >oan-miR-1385 | MIMAT0007151 |
| >hsa-miR-107 | MIMAT0000104 | >hsa-miR-513a-3p | MIMAT0004777 | >oan-miR-191 | MIMAT0007152 |
| >dme-miR-1 | MIMAT0000105 | >hsa-miR-506 | MIMAT0002878 | >oan-miR-425 | MIMAT0007154 |
| >dme-miR-2a | MIMAT0000106 | >hsa-miR-507 | MIMAT0002879 | >oan-miR-454 | MIMAT0007157 |
| >dme-miR-2b | MIMAT0000107 | >hsa-miR-508-5p | MIMAT0004778 | >oan-miR-130b | MIMAT0007159 |
| >dme-miR-3 | MIMAT0000108 | >hsa-miR-508-3p | MIMAT0002880 | >oan-miR-21 | MIMAT0007160 |
| >dme-miR-4 | MIMAT0000109 | >hsa-miR-509-5p | MIMAT0004779 | >oan-miR-1386 | MIMAT0007162 |
| >dme-miR-5 | MIMAT0000110 | >hsa-miR-509-3p | MIMAT0002881 | >oan-miR-1387 | MIMAT0007163 |
| >dme-miR-6 | MIMAT0000111 | >hsa-miR-510 | MIMAT0002882 | >oan-miR-15a | MIMAT0007164 |
| >dme-miR-7 | MIMAT0000112 | >hsa-miR-514 | MIMAT0002883 | >oan-miR-16a | MIMAT0007166 |
| >dme-miR-8 | MIMAT0000113 | >osa-miR-528 | MIMAT0002884 | >oan-miR-1388 | MIMAT0007169 |
| >dme-miR-9a | MIMAT0000114 | >osa-miR-529a | MIMAT0002885 | >oan-miR-214 | MIMAT0007171 |
| >dme-miR-10 | MIMAT0000115 | >osa-miR-530-5p | MIMAT0006787 | >oan-miR-199 | MIMAT0007173 |
| >dme-miR-11 | MIMAT0000116 | >osa-miR-530-3p | MIMAT0002886 | >oan-miR-1389 | MIMAT0007174 |
| >dme-miR-12 | MIMAT0000117 | >osa-miR-531 | MIMAT0002887 | >oan-miR-1390 | MIMAT0007176 |
| >dme-miR-13a | MIMAT0000118 | >hsa-miR-532-5p | MIMAT0002888 | >oan-miR-98 | MIMAT0007178 |
| >dme-miR-13b | MIMAT0000119 | >hsa-miR-532-3p | MIMAT0004780 | >oan-let-7f | MIMAT0007179 |
| >dme-miR-14 | MIMAT0000120 | >mmu-miR-532-5p | MIMAT0002889 | >oan-miR-802 | MIMAT0007181 |
| >mmu-let-7g | MIMAT0000121 | >mmu-miR-532-3p | MIMAT0004781 | >oan-miR-99 | MIMAT0007183 |
| >mmu-let-7i | MIMAT0000122 | >fru-let-7h | MIMAT0002892 | >oan-miR-1391 | MIMAT0007186 |
| >mmu-miR-1 | MIMAT0000123 | >tni-let-7h | MIMAT0002893 | >oan-miR-1392 | MIMAT0007187 |
| >mmu-miR-15b | MIMAT0000124 | >fru-miR-223 | MIMAT0002894 | >oan-miR-383 | MIMAT0007189 |
| >mmu-miR-23b | MIMAT0000125 | >tni-miR-223 | MIMAT0002895 | >oan-miR-1393 | MIMAT0007191 |
| >mmu-miR-27b | MIMAT0000126 | >fru-miR-124 | MIMAT0002896 | >oan-miR-135a | MIMAT0007193 |
| >mmu-miR-29b | MIMAT0000127 | >tni-miR-124 | MIMAT0002897 | >oan-miR-302 | MIMAT0007195 |
| >mmu-miR-30a | MIMAT0000128 | >fru-let-7e | MIMAT0002898 | >oan-miR-1394 | MIMAT0007196 |
| >mmu-miR-30b | MIMAT0000130 | >tni-let-7e | MIMAT0002899 | >oan-miR-1395 | MIMAT0007197 |
| >mmu-miR-99a | MIMAT0000131 | >fru-miR-219 | MIMAT0002900 | >oan-miR-1396 | MIMAT0007198 |
| >mmu-miR-99b | MIMAT0000132 | >tni-miR-219 | MIMAT0002901 | >oan-miR-1397 | MIMAT0007199 |
| >mmu-miR-101a | MIMAT0000133 | >fru-miR-101a | MIMAT0002902 | >oan-miR-1398 | MIMAT0007201 |
| >mmu-miR-124 | MIMAT0000134 | >tni-miR-101a | MIMAT0002903 | >oan-miR-1399 | MIMAT0007202 |
| >mmu-miR-125a-5p | MIMAT0000135 | >fru-miR-222 | MIMAT0002904 | >oan-miR-30e | MIMAT0007203 |
| >mmu-miR-125a-3p | MIMAT0004528 | >tni-miR-222 | MIMAT0002905 | >oan-miR-1400 | MIMAT0007206 |
| >mmu-miR-125b-5p | MIMAT0000136 | >fru-miR-221 | MIMAT0002906 | >oan-miR-1401 | MIMAT0007207 |
| >mmu-miR-126-5p | MIMAT0000137 | >tni-miR-221 | MIMAT0002907 | >oan-miR-1402 | MIMAT0007209 |
| >mmu-miR-126-3p | MIMAT0000138 | >fru-miR-142a | MIMAT0002908 | >oan-miR-1403 | MIMAT0007210 |
| >mmu-miR-127 | MIMAT0000139 | >tni-miR-142a | MIMAT0002909 | >oan-miR-1404 | MIMAT0007211 |
| >mmu-miR-128 | MIMAT0000140 | >fru-miR-9 | MIMAT0002910 | >oan-miR-1405 | MIMAT0007213 |
| >mmu-miR-130a | MIMAT0000141 | >tni-miR-9 | MIMAT0002911 | >oan-miR-1406 | MIMAT0007214 |
| >mmu-miR-9 | MIMAT0000142 | >fru-miR-23a | MIMAT0002912 | >oan-miR-138 | MIMAT0007216 |
| >mmu-miR-132 | MIMAT0000144 | >tni-miR-23a | MIMAT0002913 | >oan-miR-1407 | MIMAT0007217 |
| >mmu-miR-133a | MIMAT0000145 | >fru-miR-25 | MIMAT0002914 | >oan-miR-10b | MIMAT0007218 |
| >mmu-miR-134 | MIMAT0000146 | >tni-miR-25 | MIMAT0002915 | >oan-miR-217 | MIMAT0007221 |
| >mmu-miR-135a | MIMAT0000147 | >fru-miR-17 | MIMAT0002916 | >oan-miR-216 | MIMAT0007223 |
| >mmu-miR-136 | MIMAT0000148 | >tni-miR-17 | MIMAT0002917 | >oan-miR-1408 | MIMAT0007225 |
| >mmu-miR-137 | MIMAT0000149 | >fru-miR-18 | MIMAT0002918 | >oan-miR-1409 | MIMAT0007226 |
| >mmu-miR-138 | MIMAT0000150 | >tni-miR-18 | MIMAT0002919 | >oan-miR-1410 | MIMAT0007228 |
| >mmu-miR-140 | MIMAT0000151 | >fru-miR-19a | MIMAT0002920 | >oan-miR-1411 | MIMAT0007229 |
| >mmu-miR-141 | MIMAT0000153 | >tni-miR-19a | MIMAT0002921 | >oan-miR-146a | MIMAT0007230 |
| >mmu-miR-142-5p | MIMAT0000154 | >fru-miR-19b | MIMAT0002922 | >oan-miR-1412 | MIMAT0007232 |
| >mmu-miR-142-3p | MIMAT0000155 | >tni-miR-19b | MIMAT0002923 | >oan-miR-1413 | MIMAT0007233 |
| >mmu-miR-144 | MIMAT0000156 | >fru-miR-92 | MIMAT0002924 | >oan-let-7d | MIMAT0007234 |
| >mmu-miR-145 | MIMAT0000157 | >tni-miR-92 | MIMAT0002925 | >oan-miR-1421v | MIMAT0007236 |
| >mmu-miR-146a | MIMAT0000158 | >fru-miR-184 | MIMAT0002926 | >oan-miR-1421w | MIMAT0007239 |
| >mmu-miR-149 | MIMAT0000159 | >tni-miR-184 | MIMAT0002927 | >oan-miR-1419b | MIMAT0007240 |
| >mmu-miR-150 | MIMAT0000160 | >fru-let-7a | MIMAT0002928 | >oan-miR-1421x | MIMAT0007242 |
| >mmu-miR-151-5p | MIMAT0004536 | >tni-let-7a | MIMAT0002929 | >oan-miR-1421y | MIMAT0007244 |
| >mmu-miR-151-3p | MIMAT0000161 | >fru-miR-187 | MIMAT0002930 | >oan-miR-1421z | MIMAT0007245 |
| >mmu-miR-152 | MIMAT0000162 | >tni-miR-187 | MIMAT0002931 | >oan-miR-1419c | MIMAT0007247 |
| >mmu-miR-153 | MIMAT0000163 | >fru-miR-137 | MIMAT0002932 | >oan-miR-1421aa | MIMAT0007249 |
| >mmu-miR-154 | MIMAT0000164 | >tni-miR-137 | MIMAT0002933 | >oan-miR-1421ab | MIMAT0007251 |
| >mmu-miR-155 | MIMAT0000165 | >tni-miR-204b | MIMAT0002935 | >oan-miR-1419d | MIMAT0007253 |
| >ath-miR156a | MIMAT0000166 | >fru-miR-458 | MIMAT0002936 | >oan-miR-1414 | MIMAT0007255 |
| >ath-miR156b | MIMAT0000167 | >tni-miR-458 | MIMAT0002937 | >oan-miR-1421ac | MIMAT0007257 |
| >ath-miR156c | MIMAT0000168 | >fru-miR-489 | MIMAT0002938 | >oan-miR-1421ad | MIMAT0007259 |
| >ath-miR156d | MIMAT0000169 | >tni-miR-489 | MIMAT0002939 | >oan-miR-1421ae | MIMAT0007261 |
| >ath-miR156e | MIMAT0000170 | >dre-miR-489 | MIMAT0002940 | >oan-miR-1419e | MIMAT0007262 |
| >ath-miR156f | MIMAT0000171 | >fru-miR-192 | MIMAT0002941 | >oan-miR-1421af | MIMAT0007264 |
| >ath-miR157a | MIMAT0000172 | >tni-miR-192 | MIMAT0002942 | >oan-miR-187 | MIMAT0007267 |
| >ath-miR157b | MIMAT0000173 | >fru-miR-194 | MIMAT0002943 | >oan-miR-32 | MIMAT0007268 |
| >ath-miR157c | MIMAT0000174 | >tni-miR-194 | MIMAT0002944 | >oan-miR-1415 | MIMAT0007270 |
| >ath-miR157d | MIMAT0000175 | >fru-miR-142b | MIMAT0002945 | >oan-miR-1416 | MIMAT0007271 |
| >ath-miR158a | MIMAT0000176 | >tni-miR-142b | MIMAT0002946 | >oan-miR-873 | MIMAT0007272 |
| >ath-miR159a | MIMAT0000177 | >fru-miR-30b | MIMAT0002947 | >oan-miR-1417 | MIMAT0007274 |
| >ath-miR160a | MIMAT0000178 | >tni-miR-30b | MIMAT0002948 | >oan-miR-1418 | MIMAT0007276 |
| >ath-miR160b | MIMAT0000179 | >fru-miR-30d | MIMAT0002949 | >oan-miR-17 | MIMAT0007278 |
| >ath-miR160c | MIMAT0000180 | >tni-miR-30d | MIMAT0002950 | >osa-miR1423 | MIMAT0005957 |
| >ath-miR161.2 | MIMAT0006779 | >fru-miR-27b | MIMAT0002951 | >osa-miR1424 | MIMAT0005958 |
| >ath-miR161.1 | MIMAT0000181 | >tni-miR-27b | MIMAT0002952 | >osa-miR1425 | MIMAT0005959 |
| >ath-miR162a | MIMAT0000182 | >fru-miR-23b | MIMAT0002953 | >osa-miR1426 | MIMAT0005960 |
| >ath-miR162b | MIMAT0000183 | >tni-miR-23b | MIMAT0002954 | >osa-miR1427 | MIMAT0005961 |
| >ath-miR163 | MIMAT0000184 | >fru-miR-196a | MIMAT0002955 | >osa-miR1428a-5p | MIMAT0005962 |
| >ath-miR164a | MIMAT0000185 | >tni-miR-196a | MIMAT0002956 | >osa-miR1428a-3p | MIMAT0007280 |
| >ath-miR164b | MIMAT0000186 | >fru-miR-126 | MIMAT0002957 | >osa-miR1429 | MIMAT0005963 |
| >ath-miR165a | MIMAT0000187 | >tni-miR-126 | MIMAT0002958 | >osa-miR1430 | MIMAT0005964 |
| >ath-miR165b | MIMAT0000188 | >fru-miR-199 | MIMAT0002959 | >osa-miR1431 | MIMAT0005965 |
| >ath-miR166a | MIMAT0000189 | >tni-miR-199 | MIMAT0002960 | >osa-miR1432 | MIMAT0005966 |
| >ath-miR166b | MIMAT0000190 | >fru-miR-148 | MIMAT0002961 | >osa-miR1433 | MIMAT0005967 |
| >ath-miR166c | MIMAT0000191 | >tni-miR-148 | MIMAT0002962 | >osa-miR444b.2 | MIMAT0005968 |
| >ath-miR166d | MIMAT0000192 | >fru-miR-10b | MIMAT0002963 | >osa-miR444b.1 | MIMAT0005969 |
| >ath-miR166e | MIMAT0000193 | >tni-miR-10b | MIMAT0002964 | >osa-miR444c.2 | MIMAT0005970 |
| >ath-miR166f | MIMAT0000194 | >fru-miR-205 | MIMAT0002965 | >osa-miR444c.1 | MIMAT0005971 |
| >ath-miR166g | MIMAT0000195 | >tni-miR-205 | MIMAT0002966 | >osa-miR444d.3 | MIMAT0005972 |
| >ath-miR167a | MIMAT0000196 | >fru-miR-365 | MIMAT0002967 | >osa-miR444d.2 | MIMAT0005973 |
| >ath-miR167b | MIMAT0000197 | >tni-miR-365 | MIMAT0002968 | >osa-miR444d.1 | MIMAT0005974 |
| >ath-miR168a | MIMAT0000198 | >fru-miR-193 | MIMAT0002969 | >osa-miR444e | MIMAT0005975 |
| >ath-miR168b | MIMAT0000199 | >tni-miR-193 | MIMAT0002970 | >osa-miR444f | MIMAT0005976 |
| >ath-miR169a | MIMAT0000200 | >fru-miR-217 | MIMAT0002971 | >gga-miR-1329 | MIMAT0007281 |
| >ath-miR170 | MIMAT0000201 | >tni-miR-217 | MIMAT0002972 | >gga-miR-1397 | MIMAT0007283 |
| >ath-miR171a | MIMAT0000202 | >fru-miR-216a | MIMAT0002973 | >gga-miR-1416 | MIMAT0007285 |
| >ath-miR172a | MIMAT0000203 | >tni-miR-216a | MIMAT0002974 | >gga-miR-22 | MIMAT0007288 |
| >ath-miR172b | MIMAT0000205 | >fru-miR-216b | MIMAT0002975 | >gga-miR-551 | MIMAT0007290 |
| >ath-miR173 | MIMAT0000206 | >tni-miR-216b | MIMAT0002976 | >gga-miR-454 | MIMAT0007292 |
| >ath-miR159b | MIMAT0000207 | >fru-let-7i | MIMAT0002977 | >osa-miR810b.1 | MIMAT0005977 |
| >mmu-miR-10b | MIMAT0000208 | >tni-let-7i | MIMAT0002978 | >osa-miR810b.2 | MIMAT0005978 |
| >mmu-miR-129-5p | MIMAT0000209 | >fru-miR-429 | MIMAT0002979 | >mdv1-miR-M9 | MIMAT0005979 |
| >mmu-miR-181a | MIMAT0000210 | >tni-miR-429 | MIMAT0002980 | >mdv1-miR-M10-5p | MIMAT0007294 |
| >mmu-miR-182 | MIMAT0000211 | >fru-miR-200a | MIMAT0002981 | >mdv1-miR-M10-3p | MIMAT0005981 |
| >mmu-miR-183 | MIMAT0000212 | >tni-miR-200a | MIMAT0002982 | >mdv1-miR-M11-5p | MIMAT0005982 |
| >mmu-miR-184 | MIMAT0000213 | >fru-miR-200b | MIMAT0002983 | >mdv1-miR-M11-3p | MIMAT0005983 |
| >mmu-miR-185 | MIMAT0000214 | >tni-miR-200b | MIMAT0002984 | >mdv1-miR-M12 | MIMAT0005984 |
| >mmu-miR-186 | MIMAT0000215 | >fru-miR-218b | MIMAT0002985 | >mdv1-miR-M13 | MIMAT0005985 |
| >mmu-miR-187 | MIMAT0000216 | >tni-miR-218b | MIMAT0002986 | >gga-miR-1434 | MIMAT0007295 |
| >mmu-miR-188-5p | MIMAT0000217 | >fru-miR-301 | MIMAT0002987 | >oan-miR-1421ag | MIMAT0007298 |
| >mmu-miR-188-3p | MIMAT0004541 | >tni-miR-301 | MIMAT0002988 | >oan-miR-1421ah | MIMAT0007303 |
| >mmu-miR-24 | MIMAT0000219 | >fru-miR-130 | MIMAT0002989 | >oan-miR-1421ai | MIMAT0007305 |
| >mmu-miR-190 | MIMAT0000220 | >tni-miR-130 | MIMAT0002990 | >oan-miR-1421aj | MIMAT0007307 |
| >mmu-miR-191 | MIMAT0000221 | >fru-miR-122 | MIMAT0002991 | >oan-miR-1421ak | MIMAT0007309 |
| >hsa-miR-192 | MIMAT0000222 | >tni-miR-122 | MIMAT0002992 | >oan-miR-1421al | MIMAT0007311 |
| >mmu-miR-193 | MIMAT0000223 | >fru-miR-218a | MIMAT0002993 | >oan-miR-1421am | MIMAT0007313 |
| >mmu-miR-194 | MIMAT0000224 | >tni-miR-218a | MIMAT0002994 | >oan-miR-1422p | MIMAT0007315 |
| >mmu-miR-195 | MIMAT0000225 | >fru-let-7d | MIMAT0002995 | >oan-miR-1422q | MIMAT0007316 |
| >hsa-miR-196a | MIMAT0000226 | >tni-let-7d | MIMAT0002996 | >oan-miR-1420g | MIMAT0007318 |
| >hsa-miR-197 | MIMAT0000227 | >fru-miR-460 | MIMAT0002997 | >oan-miR-1419f | MIMAT0007321 |
| >hsa-miR-198 | MIMAT0000228 | >tni-miR-460 | MIMAT0002998 | >oan-miR-1419g | MIMAT0007322 |
| >mmu-miR-199a-5p | MIMAT0000229 | >fru-miR-21 | MIMAT0002999 | >osa-miR1435 | MIMAT0005986 |
| >mmu-miR-199a-3p | MIMAT0000230 | >tni-miR-21 | MIMAT0003000 | >osa-miR1436 | MIMAT0005987 |
| >hsa-miR-199a-5p | MIMAT0000231 | >fru-miR-153b | MIMAT0003001 | >osa-miR1437 | MIMAT0005988 |
| >hsa-miR-199a-3p | MIMAT0000232 | >tni-miR-153b | MIMAT0003002 | >osa-miR1438 | MIMAT0005989 |
| >mmu-miR-200b | MIMAT0000233 | >fru-miR-181b | MIMAT0003003 | >osa-miR1440 | MIMAT0005990 |
| >mmu-miR-201 | MIMAT0000234 | >tni-miR-181b | MIMAT0003004 | >osa-miR1441 | MIMAT0005991 |
| >mmu-miR-202-5p | MIMAT0004546 | >fru-miR-181a | MIMAT0003111 | >osa-miR1442 | MIMAT0005992 |
| >mmu-miR-202-3p | MIMAT0000235 | >tni-miR-181a | MIMAT0003050 | >osa-miR1439 | MIMAT0005993 |
| >mmu-miR-203 | MIMAT0000236 | >fru-miR-338 | MIMAT0003007 | >ptc-miR1711 | MIMAT0005994 |
| >mmu-miR-204 | MIMAT0000237 | >tni-miR-338 | MIMAT0003008 | >ptc-miR171m | MIMAT0005995 |
| >mmu-miR-205 | MIMAT0000238 | >fru-miR-7 | MIMAT0003009 | >ptc-miR171n | MIMAT0005996 |
| >mmu-miR-206 | MIMAT0000239 | >tni-miR-7 | MIMAT0003010 | >ptc-miR530a | MIMAT0005997 |
| >mmu-miR-207 | MIMAT0000240 | >fru-miR-153a | MIMAT0003011 | >ptc-miR530b | MIMAT0005998 |
| >hsa-miR-208a | MIMAT0000241 | >tni-miR-153a | MIMAT0003012 | >ptc-miR1444a | MIMAT0005999 |
| >hsa-miR-129-5p | MIMAT0000242 | >fru-let-7j | MIMAT0003013 | >ptc-miR1444b | MIMAT0006000 |
| >hsa-miR-148a | MIMAT0000243 | >tni-let-7j | MIMAT0003014 | >ptc-miR1444c | MIMAT0006001 |
| >hsa-miR-30c | MIMAT0000244 | >dre-let-7j | MIMAT0003015 | >ptc-miR1445 | MIMAT0006002 |
| >hsa-miR-30d | MIMAT0000245 | >fru-let-7b | MIMAT0003016 | >ptc-miR827 | MIMAT0006003 |
| >mmu-miR-122 | MIMAT0000246 | >tni-let-7b | MIMAT0003017 | >ptc-miR1446a | MIMAT0006004 |
| >mmu-miR-143 | MIMAT0000247 | >fru-miR-128 | MIMAT0003018 | >ptc-miR1446b | MIMAT0006005 |
| >mmu-miR-30e | MIMAT0000248 | >tni-miR-128 | MIMAT0003019 | >ptc-miR1446c | MIMAT0006006 |
| >hsa-miR-139-5p | MIMAT0000250 | >fru-miR-144 | MIMAT0003020 | >ptc-miR1446d | MIMAT0006007 |
| >hsa-miR-139-3p | MIMAT0004552 | >tni-miR-144 | MIMAT0003021 | >ptc-miR1446e | MIMAT0006008 |
| >hsa-miR-147 | MIMAT0000251 | >fru-miR-30c | MIMAT0003022 | >ptc-miR1447 | MIMAT0006009 |
| >hsa-miR-7 | MIMAT0000252 | >tni-miR-30c | MIMAT0003023 | >ptc-miR1448 | MIMAT0006010 |
| >hsa-miR-10a | MIMAT0000253 | >fru-miR-140 | MIMAT0003024 | >ptc-miR1449 | MIMAT0006011 |
| >hsa-miR-10b | MIMAT0000254 | >tni-miR-140 | MIMAT0003025 | >ptc-miR1450 | MIMAT0006012 |
| >hsa-miR-34a | MIMAT0000255 | >fru-miR-214 | MIMAT0003026 | >osa-miR827 | MIMAT0006013 |
| >hsa-miR-181a | MIMAT0000256 | >tni-miR-214 | MIMAT0003027 | >pta-miR482c | MIMAT0006014 |
| >hsa-miR-181b | MIMAT0000257 | >fru-miR-455 | MIMAT0003028 | >pta-miR482d | MIMAT0006015 |
| >hsa-miR-181c | MIMAT0000258 | >tni-miR-455 | MIMAT0003029 | >gga-miR-1451 | MIMAT0007324 |
| >hsa-miR-182 | MIMAT0000259 | >fru-miR-135b | MIMAT0003030 | >gga-miR-460b-5p | MIMAT0007326 |
| >hsa-miR-183 | MIMAT0000261 | >tni-miR-135b | MIMAT0003031 | >gga-miR-460b-3p | MIMAT0007327 |
| >hsa-miR-187 | MIMAT0000262 | >dre-miR-135b | MIMAT0003032 | >gga-miR-1452 | MIMAT0007328 |
| >hsa-miR-199b-5p | MIMAT0000263 | >fru-miR-196b | MIMAT0003033 | >gga-miR-1306 | MIMAT0007329 |
| >hsa-miR-199b-3p | MIMAT0004563 | >tni-miR-196b | MIMAT0003034 | >gga-miR-1453 | MIMAT0007330 |
| >hsa-miR-203 | MIMAT0000264 | >fru-miR-129 | MIMAT0003035 | >gga-miR-1454 | MIMAT0007331 |
| >hsa-miR-204 | MIMAT0000265 | >tni-miR-129 | MIMAT0003036 | >gga-miR-1455 | MIMAT0007332 |
| >hsa-miR-205 | MIMAT0000266 | >fru-miR-26 | MIMAT0003037 | >gga-miR-1456 | MIMAT0007333 |
| >hsa-miR-210 | MIMAT0000267 | >tni-miR-26 | MIMAT0003038 | >gga-miR-1457 | MIMAT0007335 |
| >hsa-miR-211 | MIMAT0000268 | >fru-miR-101b | MIMAT0003039 | >gga-miR-1458 | MIMAT0007336 |
| >hsa-miR-212 | MIMAT0000269 | >tni-miR-101b | MIMAT0003040 | >gga-miR-1459 | MIMAT0007337 |
| >hsa-miR-214 | MIMAT0000271 | >fru-miR-103 | MIMAT0003041 | >gga-miR-1460 | MIMAT0007338 |
| >hsa-miR-215 | MIMAT0000272 | >tni-miR-103 | MIMAT0003042 | >gga-miR-1461 | MIMAT0007339 |
| >hsa-miR-216a | MIMAT0000273 | >fru-miR-107 | MIMAT0003043 | >gga-miR-1462 | MIMAT0007340 |
| >hsa-miR-217 | MIMAT0000274 | >tni-miR-107 | MIMAT0003044 | >gga-miR-1463 | MIMAT0007341 |
| >hsa-miR-218 | MIMAT0000275 | >fru-miR-22b | MIMAT0003045 | >gga-miR-1464 | MIMAT0007342 |
| >hsa-miR-219-5p | MIMAT0000276 | >tni-miR-22b | MIMAT0003046 | >gga-miR-1465 | MIMAT0007343 |
| >hsa-miR-219-1-3p | MIMAT0004567 | >fru-miR-212 | MIMAT0003047 | >gga-miR-1466 | MIMAT0007344 |
| >hsa-miR-220a | MIMAT0000277 | >tni-miR-212 | MIMAT0003048 | >gga-miR-1467 | MIMAT0007345 |
| >hsa-miR-221 | MIMAT0000278 | >dre-miR-212 | MIMAT0003049 | >hiv1-miR-TAR-5p | MIMAT0006016 |
| >hsa-miR-222 | MIMAT0000279 | >fru-miR-135a | MIMAT0003051 | >hiv1-miR-TAR-3p | MIMAT0006017 |
| >hsa-miR-223 | MIMAT0000280 | >tni-miR-135a | MIMAT0003052 | >hsa-miR-1469 | MIMAT0007347 |
| >hsa-miR-224 | MIMAT0000281 | >fru-miR-29a | MIMAT0003053 | >hsa-miR-1470 | MIMAT0007348 |
| >cel-miR-124 | MIMAT0000282 | >tni-miR-29a | MIMAT0003054 | >hsa-miR-1471 | MIMAT0007349 |
| >cel-miR-228 | MIMAT0000283 | >fru-miR-29b | MIMAT0003055 | >ssc-miR-99b | MIMAT0006018 |
| >cel-miR-229 | MIMAT0000284 | >tni-miR-29b | MIMAT0003056 | >odi-miR-1468 | MIMAT0006019 |
| >cel-miR-230 | MIMAT0000285 | >fru-miR-133 | MIMAT0003057 | >odi-miR-1469 | MIMAT0006020 |
| >cel-miR-231 | MIMAT0000286 | >tni-miR-133 | MIMAT0003058 | >odi-miR-1470 | MIMAT0006021 |
| >cel-miR-232 | MIMAT0000287 | >fru-miR-132 | MIMAT0003059 | >odi-miR-1471 | MIMAT0006022 |
| >cel-miR-233 | MIMAT0000288 | >tni-miR-132 | MIMAT0003060 | >odi-miR-1472 | MIMAT0006023 |
| >cel-miR-234 | MIMAT0000289 | >fru-miR-125b | MIMAT0003061 | >odi-miR-1473 | MIMAT0006024 |
| >cel-miR-235 | MIMAT0000290 | >tni-miR-125b | MIMAT0003062 | >odi-miR-1474 | MIMAT0006025 |
| >cel-miR-236 | MIMAT0000291 | >fru-miR-100 | MIMAT0003063 | >odi-miR-1475 | MIMAT0006026 |
| >cel-miR-237 | MIMAT0000292 | >tni-miR-100 | MIMAT0003064 | >odi-miR-1476-5p | MIMAT0006027 |
| >cel-miR-238 | MIMAT0000293 | >fru-miR-189 | MIMAT0003384 | >odi-miR-1476-3p | MIMAT0006028 |
| >cel-miR-239a | MIMAT0000294 | >fru-miR-24 | MIMAT0003065 | >odi-miR-1477 | MIMAT0006029 |
| >cel-miR-239b | MIMAT0000295 | >tni-miR-24 | MIMAT0003066 | >odi-miR-1478 | MIMAT0006030 |
| >cel-miR-240 | MIMAT0000296 | >fru-miR-27e | MIMAT0003067 | >odi-miR-1479 | MIMAT0006031 |
| >cel-miR-241 | MIMAT0000297 | >tni-miR-27e | MIMAT0003068 | >odi-miR-1480 | MIMAT0006032 |
| >cel-miR-242 | MIMAT0000298 | >fru-miR-375 | MIMAT0003069 | >odi-miR-1481 | MIMAT0006033 |
| >cel-miR-243 | MIMAT0000299 | >tni-miR-375 | MIMAT0003070 | >odi-miR-1482 | MIMAT0006034 |
| >cel-miR-244 | MIMAT0000300 | >fru-miR-1 | MIMAT0003071 | >odi-miR-1483-5p | MIMAT0006035 |
| >cel-miR-245 | MIMAT0000301 | >tni-miR-1 | MIMAT0003072 | >odi-miR-1483-3p | MIMAT0006036 |
| >cel-miR-246 | MIMAT0000302 | >fru-miR-202 | MIMAT0003073 | >odi-miR-1484 | MIMAT0006037 |
| >cel-miR-247 | MIMAT0000303 | >tni-miR-202 | MIMAT0003074 | >odi-miR-1485 | MIMAT0006038 |
| >cel-miR-248 | MIMAT0000304 | >fru-miR-203 | MIMAT0003075 | >odi-miR-1486 | MIMAT0006039 |
| >cel-miR-249 | MIMAT0000305 | >tni-miR-203 | MIMAT0003076 | >odi-miR-1487 | MIMAT0006040 |
| >cel-miR-250 | MIMAT0000306 | >fru-miR-27c | MIMAT0003077 | >odi-miR-1489 | MIMAT0006041 |
| >cel-miR-251 | MIMAT0000307 | >tni-miR-27c | MIMAT0003078 | >odi-miR-1490a | MIMAT0006042 |
| >cel-miR-252 | MIMAT0000308 | >fru-miR-138 | MIMAT0003079 | >odi-miR-1490b | MIMAT0006043 |
| >cel-miR-253 | MIMAT0004215 | >tni-miR-138 | MIMAT0003080 | >odi-miR-1491 | MIMAT0006044 |
| >cel-miR-254 | MIMAT0000310 | >fru-miR-125a | MIMAT0003081 | >odi-miR-1492 | MIMAT0006045 |
| >cel-miR-255 | MIMAT0004216 | >tni-miR-125a | MIMAT0003082 | >odi-miR-1493 | MIMAT0006046 |
| >cel-miR-256 | MIMAT0000311 | >fru-miR-20 | MIMAT0003083 | >odi-miR-1494 | MIMAT0006047 |
| >cel-miR-257 | MIMAT0000312 | >tni-miR-20 | MIMAT0003084 | >odi-miR-1495 | MIMAT0006048 |
| >cel-miR-258 | MIMAT0000313 | >fru-miR-15b | MIMAT0003085 | >odi-miR-1496 | MIMAT0006049 |
| >cel-miR-259 | MIMAT0000314 | >tni-miR-15b | MIMAT0003086 | >odi-miR-1497a | MIMAT0006050 |
| >cel-miR-260 | MIMAT0000315 | >fru-miR-10c | MIMAT0003087 | >odi-miR-1497b | MIMAT0006051 |
| >cel-miR-261 | MIMAT0000316 | >tni-miR-10c | MIMAT0003088 | >odi-miR-1497c | MIMAT0006052 |
| >cel-miR-262 | MIMAT0000317 | >fru-miR-183 | MIMAT0003089 | >odi-miR-1497d | MIMAT0006053 |
| >hsa-miR-200b | MIMAT0000318 | >tni-miR-183 | MIMAT0003090 | >odi-miR-1497e | MIMAT0006054 |
| >dme-miR-263a | MIMAT0000319 | >fru-miR-96 | MIMAT0003091 | >odi-miR-1497f | MIMAT0006055 |
| >cel-miR-264 | MIMAT0000320 | >tni-miR-96 | MIMAT0003092 | >odi-miR-1497g | MIMAT0006056 |
| >cel-miR-265 | MIMAT0000321 | >fru-miR-182 | MIMAT0003093 | >odi-miR-1497h | MIMAT0006057 |
| >cel-miR-266 | MIMAT0000322 | >tni-miR-182 | MIMAT0003094 | >odi-miR-1498 | MIMAT0006058 |
| >cel-miR-267 | MIMAT0000323 | >fru-miR-210 | MIMAT0003095 | >odi-miR-1499 | MIMAT0006059 |
| >cel-miR-268 | MIMAT0000324 | >tni-miR-210 | MIMAT0003096 | >odi-miR-1500 | MIMAT0006060 |
| >cel-miR-269 | MIMAT0000325 | >fru-let-7g | MIMAT0003097 | >odi-miR-1501 | MIMAT0006061 |
| >cel-miR-270 | MIMAT0000326 | >tni-let-7g | MIMAT0003098 | >odi-miR-1502 | MIMAT0006062 |
| >cel-miR-271 | MIMAT0000327 | >fru-miR-22a | MIMAT0003099 | >odi-miR-1503 | MIMAT0006063 |
| >cel-miR-272 | MIMAT0000328 | >tni-miR-22a | MIMAT0003100 | >odi-miR-1504 | MIMAT0006064 |
| >cel-miR-273 | MIMAT0000329 | >fru-miR-204 | MIMAT0003101 | >odi-miR-1505 | MIMAT0006065 |
| >dme-miR-184 | MIMAT0000331 | >tni-miR-204a | MIMAT0003102 | >odi-miR-1506 | MIMAT0006066 |
| >dme-miR-274 | MIMAT0000332 | >fru-miR-152 | MIMAT0003103 | >cin-miR-1473 | MIMAT0006067 |
| >dme-miR-275 | MIMAT0000333 | >tni-miR-152 | MIMAT0003104 | >cin-miR-1497 | MIMAT0006068 |
| >dme-miR-92a | MIMAT0000334 | >fru-miR-15a | MIMAT0003105 | >csa-miR-1473 | MIMAT0006069 |
| >dme-miR-219 | MIMAT0000335 | >tni-miR-15a | MIMAT0003106 | >csa-miR-1497 | MIMAT0006070 |
| >dme-miR-276a | MIMAT0000337 | >fru-miR-16 | MIMAT0003107 | >odi-miR-1a | MIMAT0006071 |
| >dme-miR-277 | MIMAT0000338 | >tni-miR-16 | MIMAT0003108 | >odi-miR-1b | MIMAT0006072 |
| >dme-miR-278 | MIMAT0000339 | >fru-miR-190 | MIMAT0003109 | >odi-miR-1c | MIMAT0006073 |
| >dme-miR-133 | MIMAT0000340 | >tni-miR-190 | MIMAT0003110 | >odi-let-7a | MIMAT0006074 |
| >dme-miR-279 | MIMAT0000341 | >mmu-miR-489 | MIMAT0003112 | >odi-let-7b | MIMAT0006075 |
| >dme-miR-33 | MIMAT0000342 | >rno-miR-489 | MIMAT0003113 | >odi-let-7c | MIMAT0006076 |
| >dme-miR-280 | MIMAT0000343 | >rno-miR-383 | MIMAT0003114 | >odi-let-7d | MIMAT0006077 |
| >dme-miR-281 | MIMAT0000345 | >rno-miR-207 | MIMAT0003115 | >odi-miR-7 | MIMAT0006078 |
| >dme-miR-282 | MIMAT0000346 | >rno-miR-501 | MIMAT0003116 | >odi-miR-31 | MIMAT0006079 |
| >dme-miR-283 | MIMAT0000347 | >rno-miR-361 | MIMAT0003117 | >odi-miR-92a | MIMAT0006080 |
| >dme-miR-284 | MIMAT0000348 | >rno-miR-215 | MIMAT0003118 | >odi-miR-92b | MIMAT0006081 |
| >dme-miR-34 | MIMAT0000350 | >rno-miR-224 | MIMAT0003119 | >odi-miR-124a | MIMAT0006082 |
| >dme-miR-124 | MIMAT0000351 | >mmu-miR-483 | MIMAT0004782 | >odi-miR-124b | MIMAT0006083 |
| >dme-miR-79 | MIMAT0000352 | >rno-miR-483 | MIMAT0003121 | >odi-miR-219 | MIMAT0006084 |
| >dme-miR-276b | MIMAT0000354 | >rno-miR-370 | MIMAT0003122 | >odi-miR-281 | MIMAT0006085 |
| >dme-miR-210 | MIMAT0000355 | >rno-miR-377 | MIMAT0003123 | >cin-let-7a | MIMAT0006086 |
| >dme-miR-285 | MIMAT0000356 | >rno-miR-412 | MIMAT0003124 | >cin-let-7b | MIMAT0006087 |
| >dme-miR-100 | MIMAT0000357 | >rno-miR-1 | MIMAT0003125 | >cin-let-7c | MIMAT0006088 |
| >dme-miR-92b | MIMAT0000358 | >rno-miR-133b | MIMAT0003126 | >cin-let-7d | MIMAT0006089 |
| >dme-miR-286 | MIMAT0000359 | >mmu-miR-484 | MIMAT0003127 | >cin-let-7e | MIMAT0006090 |
| >dme-miR-287 | MIMAT0000360 | >mmu-miR-485 | MIMAT0003128 | >cin-miR-7 | MIMAT0006091 |
| >dme-miR-87 | MIMAT0000361 | >mmu-miR-486 | MIMAT0003130 | >cin-miR-31 | MIMAT0006092 |
| >dme-miR-263b | MIMAT0000362 | >ppt-miR390a | MIMAT0003131 | >cin-miR-33 | MIMAT0006093 |
| >dme-miR-288 | MIMAT0000363 | >ppt-miR390b | MIMAT0003132 | >cin-miR-34 | MIMAT0006094 |
| >dme-miR-289 | MIMAT0000364 | >ppt-miR319a | MIMAT0003133 | >cin-miR-78 | MIMAT0006095 |
| >dme-bantam | MIMAT0000365 | >ppt-miR319b | MIMAT0003134 | >cin-miR-92a | MIMAT0006096 |
| >mmu-miR-290-5p | MIMAT0000366 | >ppt-miR319c | MIMAT0003135 | >cin-miR-92b | MIMAT0006097 |
| >mmu-miR-290-3p | MIMAT0004572 | >ppt-miR319d | MIMAT0003136 | >cin-miR-92c | MIMAT0006098 |
| >mmu-miR-291a-5p | MIMAT0000367 | >ppt-miR533a | MIMAT0004783 | >cin-miR-101 | MIMAT0006099 |
| >mmu-miR-291a-3p | MIMAT0000368 | >ppt-miR534a | MIMAT0003138 | >cin-miR-124 | MIMAT0006100 |
| >mmu-miR-292-5p | MIMAT0000369 | >ppt-miR535a | MIMAT0003139 | >cin-miR-126 | MIMAT0006101 |
| >mmu-miR-292-3p | MIMAT0000370 | >ppt-miR535b | MIMAT0003140 | >cin-miR-133 | MIMAT0006102 |
| >mmu-miR-293 | MIMAT0000371 | >ppt-miR535c | MIMAT0003141 | >cin-miR-141 | MIMAT0006103 |
| >mmu-miR-294 | MIMAT0000372 | >osa-miR535 | MIMAT0003142 | >cin-miR-153 | MIMAT0006104 |
| >mmu-miR-295 | MIMAT0000373 | >ppt-miR156a | MIMAT0003143 | >cin-miR-155 | MIMAT0006105 |
| >mmu-miR-296-5p | MIMAT0000374 | >ppt-miR536a | MIMAT0003144 | >cin-miR-181 | MIMAT0006106 |
| >mmu-miR-296-3p | MIMAT0004576 | >ppt-miR537a | MIMAT0003145 | >cin-miR-183 | MIMAT0006107 |
| >mmu-miR-297a | MIMAT0000375 | >ppt-miR537b | MIMAT0003146 | >cin-miR-184 | MIMAT0006108 |
| >mmu-miR-298 | MIMAT0000376 | >ppt-miR538a | MIMAT0003147 | >cin-miR-199-3p | MIMAT0006109 |
| >mmu-miR-299 | MIMAT0004577 | >ppt-miR538b | MIMAT0003148 | >cin-miR-200 | MIMAT0006110 |
| >mmu-miR-300 | MIMAT0000378 | >ppt-miR538c | MIMAT0003149 | >cin-miR-216 | MIMAT0006111 |
| >mmu-miR-301a | MIMAT0000379 | >hsa-miR-455-5p | MIMAT0003150 | >cin-miR-217 | MIMAT0006112 |
| >mmu-miR-302a | MIMAT0000380 | >hsa-miR-455-3p | MIMAT0004784 | >cin-miR-219 | MIMAT0006113 |
| >mmu-miR-34c | MIMAT0000381 | >hsa-miR-539 | MIMAT0003163 | >cin-miR-281 | MIMAT0006114 |
| >mmu-miR-34b-5p | MIMAT0000382 | >hsa-miR-544 | MIMAT0003164 | >cin-miR-672 | MIMAT0006115 |
| >mmu-miR-34b-3p | MIMAT0004581 | >hsa-miR-545 | MIMAT0003165 | >csa-let-7a | MIMAT0006116 |
| >mmu-let-7d | MIMAT0000383 | >mmu-miR-546 | MIMAT0003166 | >csa-let-7b | MIMAT0006117 |
| >mmu-miR-106a | MIMAT0000385 | >mmu-miR-540-5p | MIMAT0004786 | >csa-let-7c | MIMAT0006118 |
| >mmu-miR-106b | MIMAT0000386 | >mmu-miR-540-3p | MIMAT0003167 | >csa-let-7d | MIMAT0006119 |
| >mmu-miR-130b | MIMAT0000387 | >mmu-miR-543 | MIMAT0003168 | >csa-miR-7 | MIMAT0006120 |
| >dme-miR-303 | MIMAT0000388 | >mmu-miR-539 | MIMAT0003169 | >csa-miR-31 | MIMAT0006121 |
| >dme-miR-31b | MIMAT0000389 | >mmu-miR-541 | MIMAT0003170 | >csa-miR-34 | MIMAT0006122 |
| >dme-miR-304 | MIMAT0000390 | >mmu-miR-542-5p | MIMAT0003171 | >csa-miR-92a | MIMAT0006123 |
| >dme-miR-305 | MIMAT0000391 | >mmu-miR-542-3p | MIMAT0003172 | >csa-miR-92b | MIMAT0006124 |
| >dme-miR-9c | MIMAT0000392 | >mmu-miR-547 | MIMAT0003173 | >csa-miR-92c | MIMAT0006125 |
| >dme-miR-306 | MIMAT0000393 | >rno-miR-540 | MIMAT0003174 | >csa-miR-124 | MIMAT0006126 |
| >dme-miR-9b | MIMAT0000395 | >rno-miR-543 | MIMAT0004787 | >csa-miR-126 | MIMAT0006127 |
| >dme-let-7 | MIMAT0000396 | >rno-miR-539 | MIMAT0003176 | >csa-miR-133 | MIMAT0006128 |
| >dme-miR-125 | MIMAT0000397 | >rno-miR-541 | MIMAT0003177 | >csa-miR-141 | MIMAT0006129 |
| >dme-miR-307 | MIMAT0000398 | >rno-miR-542-5p | MIMAT0003178 | >csa-miR-153 | MIMAT0006130 |
| >dme-miR-308 | MIMAT0000399 | >rno-miR-542-3p | MIMAT0003179 | >csa-miR-155 | MIMAT0006131 |
| >dme-miR-31a | MIMAT0000400 | >hsa-miR-487b | MIMAT0003180 | >csa-miR-183 | MIMAT0006132 |
| >dme-miR-309 | MIMAT0000401 | >mmu-miR-367 | MIMAT0003181 | >csa-miR-200 | MIMAT0006133 |
| >dme-miR-310 | MIMAT0000402 | >mmu-miR-494 | MIMAT0003182 | >csa-miR-216a | MIMAT0006134 |
| >dme-miR-311 | MIMAT0000403 | >mmu-miR-376c | MIMAT0003183 | >csa-miR-216b | MIMAT0006135 |
| >dme-miR-312 | MIMAT0000404 | >mmu-miR-487b | MIMAT0003184 | >csa-miR-217 | MIMAT0006136 |
| >dme-miR-313 | MIMAT0000405 | >mmu-miR-369-5p | MIMAT0003185 | >csa-miR-219 | MIMAT0006137 |
| >dme-miR-314 | MIMAT0000406 | >mmu-miR-369-3p | MIMAT0003186 | >csa-miR-281 | MIMAT0006138 |
| >dme-miR-315 | MIMAT0000407 | >mmu-miR-20b | MIMAT0003187 | >gma-miR159b | MIMAT0007351 |
| >dme-miR-316 | MIMAT0000408 | >mmu-miR-450a-3p | MIMAT0004789 | >gma-miR159c | MIMAT0007352 |
| >dme-miR-317 | MIMAT0000409 | >mmu-miR-503 | MIMAT0003188 | >gma-miR162 | MIMAT0007353 |
| >dme-miR-318 | MIMAT0000410 | >mmu-miR-291b-5p | MIMAT0003189 | >gma-miR164 | MIMAT0007354 |
| >dme-miR-2c | MIMAT0000411 | >mmu-miR-291b-3p | MIMAT0003190 | >gma-miR167c | MIMAT0007355 |
| >dme-miR-iab-4-5p | MIMAT0000412 | >rno-miR-493 | MIMAT0003191 | >gma-miR169b | MIMAT0007356 |
| >dme-miR-iab-4-3p | MIMAT0000413 | >rno-miR-379 | MIMAT0003192 | >gma-miR169c | MIMAT0007357 |
| >hsa-let-7g | MIMAT0000414 | >rno-miR-494 | MIMAT0003193 | >gma-miR171a | MIMAT0007358 |
| >hsa-let-7i | MIMAT0000415 | >rno-miR-376c | MIMAT0003194 | >gma-miR390a-5p | MIMAT0007359 |
| >hsa-miR-1 | MIMAT0000416 | >rno-miR-376b-5p | MIMAT0003195 | >gma-miR390a-3p | MIMAT0007360 |
| >hsa-miR-15b | MIMAT0000417 | >rno-miR-376b-3p | MIMAT0003196 | >gma-miR390b | MIMAT0007361 |
| >hsa-miR-23b | MIMAT0000418 | >rno-miR-376a | MIMAT0003198 | >gma-miR393 | MIMAT0007362 |
| >hsa-miR-27b | MIMAT0000419 | >rno-miR-381 | MIMAT0003199 | >gma-miR171b | MIMAT0007363 |
| >hsa-miR-30b | MIMAT0000420 | >rno-miR-487b | MIMAT0003200 | >gma-miR482 | MIMAT0007364 |
| >hsa-miR-122 | MIMAT0000421 | >rno-miR-382 | MIMAT0003201 | >gma-miR1507 | MIMAT0007365 |
| >hsa-miR-124 | MIMAT0000422 | >rno-miR-485 | MIMAT0003203 | >gma-miR1508 | MIMAT0007366 |
| >hsa-miR-125b | MIMAT0000423 | >rno-miR-409-5p | MIMAT0003204 | >gma-miR1509 | MIMAT0007367 |
| >hsa-miR-128 | MIMAT0000424 | >rno-miR-409-3p | MIMAT0003205 | >gma-miR1510 | MIMAT0007368 |
| >hsa-miR-130a | MIMAT0000425 | >rno-miR-369-5p | MIMAT0003206 | >gma-miR1511 | MIMAT0007369 |
| >hsa-miR-132 | MIMAT0000426 | >rno-miR-369-3p | MIMAT0003207 | >gma-miR1512 | MIMAT0007370 |
| >hsa-miR-133a | MIMAT0000427 | >rno-miR-374 | MIMAT0003208 | >gma-miR1513 | MIMAT0007371 |
| >hsa-miR-135a | MIMAT0000428 | >rno-miR-363 | MIMAT0003210 | >gma-miR1514a | MIMAT0007372 |
| >hsa-miR-137 | MIMAT0000429 | >rno-miR-20b-5p | MIMAT0003211 | >gma-miR1514b | MIMAT0007373 |
| >hsa-miR-138 | MIMAT0000430 | >rno-miR-20b-3p | MIMAT0003212 | >gma-miR1515 | MIMAT0007374 |
| >hsa-miR-140-5p | MIMAT0000431 | >rno-miR-503 | MIMAT0003213 | >gma-miR1516 | MIMAT0007375 |
| >hsa-miR-140-3p | MIMAT0004597 | >hsa-miR-551a | MIMAT0003214 | >gma-miR1517 | MIMAT0007376 |
| >hsa-miR-141 | MIMAT0000432 | >hsa-miR-552 | MIMAT0003215 | >gma-miR1518 | MIMAT0007377 |
| >hsa-miR-142-5p | MIMAT0000433 | >hsa-miR-553 | MIMAT0003216 | >gma-miR1519 | MIMAT0007378 |
| >hsa-miR-142-3p | MIMAT0000434 | >hsa-miR-554 | MIMAT0003217 | >gma-miR1520d | MIMAT0007379 |
| >hsa-miR-143 | MIMAT0000435 | >hsa-miR-92b | MIMAT0003218 | >gma-miR1536 | MIMAT0007380 |
| >hsa-miR-144 | MIMAT0000436 | >hsa-miR-555 | MIMAT0003219 | >gma-miR1520a | MIMAT0007381 |
| >hsa-miR-145 | MIMAT0000437 | >hsa-miR-556-5p | MIMAT0003220 | >gma-miR1521 | MIMAT0007382 |
| >hsa-miR-152 | MIMAT0000438 | >hsa-miR-556-3p | MIMAT0004793 | >gma-miR1522 | MIMAT0007383 |
| >hsa-miR-153 | MIMAT0000439 | >hsa-miR-557 | MIMAT0003221 | >gma-miR1523 | MIMAT0007384 |
| >hsa-miR-191 | MIMAT0000440 | >hsa-miR-558 | MIMAT0003222 | >gma-miR1524 | MIMAT0007385 |
| >hsa-miR-9 | MIMAT0000441 | >hsa-miR-559 | MIMAT0003223 | >gma-miR1525 | MIMAT0007386 |
| >hsa-miR-125a-5p | MIMAT0000443 | >hsa-miR-561 | MIMAT0003225 | >gma-miR1520b | MIMAT0007387 |
| >hsa-miR-125a-3p | MIMAT0004602 | >hsa-miR-562 | MIMAT0003226 | >gma-miR1526 | MIMAT0007388 |
| >hsa-miR-126 | MIMAT0000445 | >hsa-miR-563 | MIMAT0003227 | >gma-miR1527 | MIMAT0007389 |
| >hsa-miR-127-5p | MIMAT0004604 | >hsa-miR-564 | MIMAT0003228 | >gma-miR1528 | MIMAT0007390 |
| >hsa-miR-127-3p | MIMAT0000446 | >hsa-miR-566 | MIMAT0003230 | >gma-miR1529 | MIMAT0007391 |
| >hsa-miR-129-3p | MIMAT0004605 | >hsa-miR-567 | MIMAT0003231 | >gma-miR1530 | MIMAT0007392 |
| >hsa-miR-134 | MIMAT0000447 | >hsa-miR-568 | MIMAT0003232 | >gma-miR1531 | MIMAT0007393 |
| >hsa-miR-136 | MIMAT0000448 | >hsa-miR-551b | MIMAT0003233 | >gma-miR1532 | MIMAT0007394 |
| >hsa-miR-146a | MIMAT0000449 | >hsa-miR-569 | MIMAT0003234 | >gma-miR1520c | MIMAT0007395 |
| >hsa-miR-149 | MIMAT0000450 | >hsa-miR-570 | MIMAT0003235 | >gma-miR1533 | MIMAT0007396 |
| >hsa-miR-150 | MIMAT0000451 | >hsa-miR-571 | MIMAT0003236 | >gma-miR1534 | MIMAT0007397 |
| >hsa-miR-154 | MIMAT0000452 | >hsa-miR-572 | MIMAT0003237 | >gma-miR1535 | MIMAT0007398 |
| >hsa-miR-184 | MIMAT0000454 | >hsa-miR-573 | MIMAT0003238 | >hsa-miR-1537 | MIMAT0007399 |
| >hsa-miR-185 | MIMAT0000455 | >hsa-miR-574-5p | MIMAT0004795 | >hsa-miR-1538 | MIMAT0007400 |
| >hsa-miR-186 | MIMAT0000456 | >hsa-miR-574-3p | MIMAT0003239 | >hsa-miR-1539 | MIMAT0007401 |
| >hsa-miR-188-5p | MIMAT0000457 | >hsa-miR-575 | MIMAT0003240 | >hsa-miR-103-as | MIMAT0007402 |
| >hsa-miR-188-3p | MIMAT0004613 | >hsa-miR-576-5p | MIMAT0003241 | >mdo-miR-1540 | MIMAT0006139 |
| >hsa-miR-190 | MIMAT0000458 | >hsa-miR-576-3p | MIMAT0004796 | >mdo-miR-1541 | MIMAT0006140 |
| >hsa-miR-193a-5p | MIMAT0004614 | >hsa-miR-577 | MIMAT0003242 | >mdo-miR-1542 | MIMAT0006141 |
| >hsa-miR-193a-3p | MIMAT0000459 | >hsa-miR-578 | MIMAT0003243 | >mdo-miR-1543 | MIMAT0006142 |
| >hsa-miR-194 | MIMAT0000460 | >hsa-miR-579 | MIMAT0003244 | >mdo-miR-340 | MIMAT0006143 |
| >hsa-miR-195 | MIMAT0000461 | >hsa-miR-580 | MIMAT0003245 | >mdo-miR-1544 | MIMAT0006144 |
| >hsa-miR-206 | MIMAT0000462 | >hsa-miR-581 | MIMAT0003246 | >mdo-miR-1545 | MIMAT0006145 |
| >cbr-let-7 | MIMAT0000463 | >hsa-miR-582-5p | MIMAT0003247 | >mdo-miR-1546 | MIMAT0006146 |
| >cbr-lin-4 | MIMAT0000464 | >hsa-miR-582-3p | MIMAT0004797 | >mdo-miR-1547 | MIMAT0006147 |
| >cbr-miR-1 | MIMAT0000465 | >hsa-miR-583 | MIMAT0003248 | >mdo-miR-1548 | MIMAT0006148 |
| >cbr-miR-34 | MIMAT0000466 | >hsa-miR-584 | MIMAT0003249 | >mdo-miR-1549 | MIMAT0006149 |
| >cbr-miR-42 | MIMAT0000467 | >hsa-miR-585 | MIMAT0003250 | >gga-miR-1550-5p | MIMAT0007403 |
| >cbr-miR-43 | MIMAT0000468 | >hsa-miR-548a-3p | MIMAT0003251 | >gga-miR-1550-3p | MIMAT0007404 |
| >cbr-miR-44 | MIMAT0000469 | >hsa-miR-586 | MIMAT0003252 | >gga-miR-1551 | MIMAT0007405 |
| >cbr-miR-45 | MIMAT0000470 | >hsa-miR-587 | MIMAT0003253 | >gga-miR-1552-5p | MIMAT0007407 |
| >cbr-miR-46 | MIMAT0000471 | >hsa-miR-548b-5p | MIMAT0004798 | >gga-miR-1552-3p | MIMAT0007408 |
| >cbr-miR-47 | MIMAT0000472 | >hsa-miR-548b-3p | MIMAT0003254 | >gga-miR-1553 | MIMAT0007410 |
| >cbr-miR-48 | MIMAT0000473 | >hsa-miR-588 | MIMAT0003255 | >gga-miR-1554 | MIMAT0007411 |
| >cbr-miR-49 | MIMAT0000474 | >hsa-miR-589 | MIMAT0004799 | >gga-miR-1555 | MIMAT0007412 |
| >cbr-miR-50 | MIMAT0000475 | >hsa-miR-550 | MIMAT0004800 | >gga-miR-1556 | MIMAT0007413 |
| >cbr-miR-52 | MIMAT0000476 | >hsa-miR-590-5p | MIMAT0003258 | >gga-miR-1557 | MIMAT0007414 |
| >cbr-miR-57 | MIMAT0000477 | >hsa-miR-590-3p | MIMAT0004801 | >gga-miR-1558 | MIMAT0007415 |
| >cbr-miR-58 | MIMAT0000478 | >hsa-miR-591 | MIMAT0003259 | >gga-miR-1559 | MIMAT0007416 |
| >cbr-miR-60 | MIMAT0000479 | >hsa-miR-592 | MIMAT0003260 | >gga-miR-1560 | MIMAT0007418 |
| >cbr-miR-67 | MIMAT0000480 | >hsa-miR-593 | MIMAT0004802 | >gga-miR-1561 | MIMAT0007419 |
| >cbr-miR-71 | MIMAT0000481 | >hsa-miR-595 | MIMAT0003263 | >gga-miR-1562 | MIMAT0007420 |
| >cbr-miR-73 | MIMAT0000482 | >hsa-miR-596 | MIMAT0003264 | >gga-miR-1563 | MIMAT0007422 |
| >cbr-miR-74 | MIMAT0000483 | >hsa-miR-597 | MIMAT0003265 | >gga-miR-1564 | MIMAT0007423 |
| >cbr-miR-75 | MIMAT0000484 | >hsa-miR-598 | MIMAT0003266 | >gga-miR-1565 | MIMAT0007425 |
| >cbr-miR-77 | MIMAT0000485 | >hsa-miR-599 | MIMAT0003267 | >gga-miR-1566 | MIMAT0007426 |
| >cbr-miR-79 | MIMAT0000486 | >hsa-miR-548a-5p | MIMAT0004803 | >gga-miR-1567 | MIMAT0007427 |
| >cbr-miR-80 | MIMAT0000487 | >hsa-miR-600 | MIMAT0003268 | >gga-miR-1568 | MIMAT0007428 |
| >cbr-miR-81 | MIMAT0000488 | >hsa-miR-601 | MIMAT0003269 | >gga-miR-1569 | MIMAT0007429 |
| >cbr-miR-82 | MIMAT0000489 | >hsa-miR-602 | MIMAT0003270 | >gga-miR-1570 | MIMAT0007430 |
| >cbr-miR-85 | MIMAT0000490 | >hsa-miR-603 | MIMAT0003271 | >gga-miR-1571 | MIMAT0007431 |
| >cbr-miR-86 | MIMAT0000491 | >hsa-miR-604 | MIMAT0003272 | >gga-miR-1572 | MIMAT0007432 |
| >cbr-miR-87 | MIMAT0000492 | >hsa-miR-605 | MIMAT0003273 | >gga-miR-1573 | MIMAT0007433 |
| >cbr-miR-90 | MIMAT0000493 | >hsa-miR-606 | MIMAT0003274 | >gga-miR-1574 | MIMAT0007435 |
| >cbr-miR-124 | MIMAT0000494 | >hsa-miR-607 | MIMAT0003275 | >gga-miR-1575 | MIMAT0007436 |
| >cbr-miR-228 | MIMAT0000495 | >hsa-miR-608 | MIMAT0003276 | >gga-miR-1576 | MIMAT0007437 |
| >cbr-miR-230 | MIMAT0000496 | >hsa-miR-609 | MIMAT0003277 | >gga-miR-1577 | MIMAT0007438 |
| >cbr-miR-232 | MIMAT0000497 | >hsa-miR-610 | MIMAT0003278 | >gga-miR-1578 | MIMAT0007439 |
| >cbr-miR-233 | MIMAT0000498 | >hsa-miR-611 | MIMAT0003279 | >gga-miR-1579 | MIMAT0007440 |
| >cbr-miR-234 | MIMAT0000499 | >hsa-miR-612 | MIMAT0003280 | >gga-miR-1580 | MIMAT0007441 |
| >cbr-miR-236 | MIMAT0000500 | >hsa-miR-613 | MIMAT0003281 | >gga-miR-1581 | MIMAT0007442 |
| >cbr-miR-241 | MIMAT0000501 | >hsa-miR-614 | MIMAT0003282 | >gga-miR-1582 | MIMAT0007443 |
| >cbr-miR-244 | MIMAT0000502 | >hsa-miR-615-5p | MIMAT0004804 | >gga-miR-1583 | MIMAT0007444 |
| >cbr-miR-245 | MIMAT0000503 | >hsa-miR-615-3p | MIMAT0003283 | >gga-miR-1584 | MIMAT0007445 |
| >cbr-miR-248 | MIMAT0000504 | >hsa-miR-616 | MIMAT0004805 | >gga-miR-1354 | MIMAT0007446 |
| >cbr-miR-250 | MIMAT0000505 | >hsa-miR-548c-5p | MIMAT0004806 | >gga-miR-1585 | MIMAT0007447 |
| >cbr-miR-251 | MIMAT0000506 | >hsa-miR-548c-3p | MIMAT0003285 | >gga-miR-1586 | MIMAT0007448 |
| >cbr-miR-252 | MIMAT0000507 | >hsa-miR-617 | MIMAT0003286 | >gga-miR-1587 | MIMAT0007449 |
| >cbr-miR-259 | MIMAT0000508 | >hsa-miR-618 | MIMAT0003287 | >gga-miR-1588 | MIMAT0007450 |
| >cbr-miR-268 | MIMAT0000509 | >hsa-miR-619 | MIMAT0003288 | >gga-miR-1589 | MIMAT0007451 |
| >hsa-miR-320a | MIMAT0000510 | >hsa-miR-620 | MIMAT0003289 | >gga-miR-1590 | MIMAT0007452 |
| >ath-miR319a | MIMAT0000511 | >hsa-miR-621 | MIMAT0003290 | >gga-miR-1591 | MIMAT0007454 |
| >ath-miR319b | MIMAT0000512 | >hsa-miR-622 | MIMAT0003291 | >gga-miR-1592 | MIMAT0007455 |
| >mmu-miR-19b | MIMAT0000513 | >hsa-miR-623 | MIMAT0003292 | >gga-miR-1593 | MIMAT0007456 |
| >mmu-miR-30c | MIMAT0000514 | >hsa-miR-624 | MIMAT0004807 | >gga-miR-1594 | MIMAT0007457 |
| >mmu-miR-30d | MIMAT0000515 | >hsa-miR-625 | MIMAT0003294 | >gga-miR-1595 | MIMAT0007458 |
| >mmu-miR-148a | MIMAT0000516 | >hsa-miR-626 | MIMAT0003295 | >gga-miR-1596 | MIMAT0007460 |
| >mmu-miR-192 | MIMAT0000517 | >hsa-miR-627 | MIMAT0003296 | >gga-miR-1597 | MIMAT0007463 |
| >mmu-miR-196a | MIMAT0000518 | >hsa-miR-628-5p | MIMAT0004809 | >gga-miR-1598 | MIMAT0007464 |
| >mmu-miR-200a | MIMAT0000519 | >hsa-miR-628-3p | MIMAT0003297 | >gga-miR-1599 | MIMAT0007465 |
| >mmu-miR-208a | MIMAT0000520 | >hsa-miR-629 | MIMAT0004810 | >gga-miR-1600 | MIMAT0007466 |
| >mmu-let-7a | MIMAT0000521 | >hsa-miR-630 | MIMAT0003299 | >gga-miR-1601 | MIMAT0007467 |
| >mmu-let-7b | MIMAT0000522 | >hsa-miR-631 | MIMAT0003300 | >gga-miR-1602 | MIMAT0007468 |
| >mmu-let-7c | MIMAT0000523 | >hsa-miR-33b | MIMAT0003301 | >gga-miR-1603 | MIMAT0007469 |
| >mmu-let-7e | MIMAT0000524 | >hsa-miR-632 | MIMAT0003302 | >gga-miR-1604 | MIMAT0007470 |
| >mmu-let-7f | MIMAT0000525 | >hsa-miR-633 | MIMAT0003303 | >gga-miR-1605 | MIMAT0007471 |
| >mmu-miR-15a | MIMAT0000526 | >hsa-miR-634 | MIMAT0003304 | >gga-miR-1606 | MIMAT0007472 |
| >mmu-miR-16 | MIMAT0000527 | >hsa-miR-635 | MIMAT0003305 | >gga-miR-1607 | MIMAT0007473 |
| >mmu-miR-18a | MIMAT0000528 | >hsa-miR-636 | MIMAT0003306 | >gga-miR-1608 | MIMAT0007474 |
| >mmu-miR-20a | MIMAT0000529 | >hsa-miR-637 | MIMAT0003307 | >gga-miR-1609 | MIMAT0007475 |
| >mmu-miR-21 | MIMAT0000530 | >hsa-miR-638 | MIMAT0003308 | >gga-miR-1610 | MIMAT0007476 |
| >mmu-miR-22 | MIMAT0000531 | >hsa-miR-639 | MIMAT0003309 | >gga-miR-1611 | MIMAT0007477 |
| >mmu-miR-23a | MIMAT0000532 | >hsa-miR-640 | MIMAT0003310 | >gga-miR-1612 | MIMAT0007478 |
| >mmu-miR-26a | MIMAT0000533 | >hsa-miR-641 | MIMAT0003311 | >gga-miR-1613 | MIMAT0007479 |
| >mmu-miR-26b | MIMAT0000534 | >hsa-miR-642 | MIMAT0003312 | >gga-miR-1614 | MIMAT0007480 |
| >mmu-miR-29a | MIMAT0000535 | >hsa-miR-643 | MIMAT0003313 | >gga-miR-1615 | MIMAT0007482 |
| >mmu-miR-29c | MIMAT0000536 | >hsa-miR-644 | MIMAT0003314 | >gga-miR-1616 | MIMAT0007483 |
| >mmu-miR-27a | MIMAT0000537 | >hsa-miR-645 | MIMAT0003315 | >gga-miR-1617 | MIMAT0007484 |
| >mmu-miR-31 | MIMAT0000538 | >hsa-miR-646 | MIMAT0003316 | >gga-miR-1618 | MIMAT0007485 |
| >mmu-miR-92a | MIMAT0000539 | >hsa-miR-647 | MIMAT0003317 | >gga-miR-1619 | MIMAT0007486 |
| >mmu-miR-93 | MIMAT0000540 | >hsa-miR-648 | MIMAT0003318 | >gga-miR-1620 | MIMAT0007487 |
| >mmu-miR-96 | MIMAT0000541 | >hsa-miR-649 | MIMAT0003319 | >gga-miR-1621 | MIMAT0007488 |
| >mmu-miR-34a | MIMAT0000542 | >hsa-miR-650 | MIMAT0003320 | >gga-miR-1622 | MIMAT0007490 |
| >mmu-miR-129-3p | MIMAT0000544 | >hsa-miR-651 | MIMAT0003321 | >gga-miR-1623 | MIMAT0007491 |
| >mmu-miR-98 | MIMAT0000545 | >hsa-miR-652 | MIMAT0003322 | >gga-miR-1624 | MIMAT0007492 |
| >mmu-miR-103 | MIMAT0000546 | >hsa-miR-548d-5p | MIMAT0004812 | >gga-miR-1625 | MIMAT0007493 |
| >rno-miR-322 | MIMAT0001619 | >hsa-miR-548d-3p | MIMAT0003323 | >gga-miR-1626 | MIMAT0007496 |
| >mmu-miR-322 | MIMAT0000548 | >hsa-miR-661 | MIMAT0003324 | >gga-miR-1627 | MIMAT0007498 |
| >rno-miR-323 | MIMAT0000550 | >hsa-miR-662 | MIMAT0003325 | >gga-miR-1628 | MIMAT0007499 |
| >mmu-miR-323-5p | MIMAT0004638 | >hsa-miR-663 | MIMAT0003326 | >gga-miR-1629 | MIMAT0007500 |
| >mmu-miR-323-3p | MIMAT0000551 | >hsa-miR-449b | MIMAT0003327 | >gga-miR-1630 | MIMAT0007501 |
| >rno-miR-301a | MIMAT0000552 | >hsa-miR-653 | MIMAT0003328 | >gga-miR-1631 | MIMAT0007502 |
| >rno-miR-324-5p | MIMAT0000553 | >hsa-miR-411 | MIMAT0003329 | >gga-miR-1c | MIMAT0007503 |
| >rno-miR-324-3p | MIMAT0000554 | >hsa-miR-654-5p | MIMAT0003330 | >gga-miR-1632 | MIMAT0007504 |
| >mmu-miR-324-5p | MIMAT0000555 | >hsa-miR-654-3p | MIMAT0004814 | >gga-miR-1633 | MIMAT0007506 |
| >mmu-miR-324-3p | MIMAT0000556 | >hsa-miR-655 | MIMAT0003331 | >gga-miR-1634 | MIMAT0007507 |
| >rno-miR-325-5p | MIMAT0000557 | >hsa-miR-656 | MIMAT0003332 | >gga-miR-1635 | MIMAT0007508 |
| >rno-miR-325-3p | MIMAT0004639 | >hsa-miR-549 | MIMAT0003333 | >gga-miR-1636 | MIMAT0007509 |
| >mmu-miR-325 | MIMAT0004640 | >hsa-miR-657 | MIMAT0003335 | >gga-miR-1637 | MIMAT0007510 |
| >mmu-miR-326 | MIMAT0000559 | >hsa-miR-658 | MIMAT0003336 | >gga-miR-1638 | MIMAT0007511 |
| >rno-miR-326 | MIMAT0000560 | >hsa-miR-659 | MIMAT0003337 | >gga-miR-1815 | MIMAT0007512 |
| >rno-miR-327 | MIMAT0000561 | >hsa-miR-660 | MIMAT0003338 | >gga-miR-1814 | MIMAT0007513 |
| >rno-let-7d | MIMAT0000562 | >hsa-miR-421 | MIMAT0003339 | >gga-miR-1639 | MIMAT0007514 |
| >rno-miR-328 | MIMAT0000564 | >hsa-miR-542-5p | MIMAT0003340 | >gga-miR-1640 | MIMAT0007515 |
| >mmu-miR-328 | MIMAT0000565 | >hsa-miR-542-3p | MIMAT0003389 | >gga-miR-1641 | MIMAT0007516 |
| >rno-miR-329 | MIMAT0000566 | >hcmv-miR-US4 | MIMAT0003341 | >gga-miR-1642 | MIMAT0007517 |
| >mmu-miR-329 | MIMAT0000567 | >hcmv-miR-UL70-5p | MIMAT0003342 | >gga-miR-1643 | MIMAT0007519 |
| >rno-miR-330 | MIMAT0004641 | >hcmv-miR-UL70-3p | MIMAT0003343 | >gga-miR-1644 | MIMAT0007520 |
| >mmu-miR-330 | MIMAT0004642 | >sv40-miR-S1-5p | MIMAT0003344 | >gga-miR-1645 | MIMAT0007521 |
| >rno-miR-331 | MIMAT0000570 | >sv40-miR-S1-3p | MIMAT0003345 | >gga-miR-1646 | MIMAT0007522 |
| >mmu-miR-331-5p | MIMAT0004643 | >dre-miR-34b | MIMAT0003346 | >gga-miR-1647 | MIMAT0007523 |
| >mmu-miR-331-3p | MIMAT0000571 | >dre-miR-31 | MIMAT0003347 | >gga-miR-1648 | MIMAT0007524 |
| >rno-miR-140 | MIMAT0000573 | >dre-miR-135a | MIMAT0003348 | >gga-miR-1649 | MIMAT0007526 |
| >rno-miR-335 | MIMAT0000575 | >dre-miR-139 | MIMAT0003349 | >gga-miR-1650 | MIMAT0007528 |
| >rno-miR-336 | MIMAT0000576 | >gga-miR-146b | MIMAT0003351 | >gga-miR-1651 | MIMAT0007529 |
| >rno-miR-337 | MIMAT0000577 | >gga-miR-147 | MIMAT0003352 | >gga-miR-1652 | MIMAT0007531 |
| >mmu-miR-337-5p | MIMAT0004644 | >gga-miR-193b | MIMAT0003353 | >gga-miR-1653 | MIMAT0007532 |
| >mmu-miR-337-3p | MIMAT0000578 | >gga-miR-202 | MIMAT0003355 | >gga-miR-1654 | MIMAT0007533 |
| >rno-miR-148b-5p | MIMAT0004645 | >gga-miR-302b | MIMAT0003357 | >gga-miR-1655 | MIMAT0007534 |
| >rno-miR-148b-3p | MIMAT0000579 | >gga-miR-302c | MIMAT0003359 | >gga-miR-1656 | MIMAT0007535 |
| >mmu-miR-148b | MIMAT0000580 | >gga-miR-302d | MIMAT0003360 | >gga-miR-135b | MIMAT0007536 |
| >rno-miR-338 | MIMAT0000581 | >gga-miR-365 | MIMAT0003361 | >gga-miR-1657 | MIMAT0007537 |
| >mmu-miR-338-5p | MIMAT0004647 | >gga-miR-375 | MIMAT0003362 | >gga-miR-1658 | MIMAT0007538 |
| >mmu-miR-338-3p | MIMAT0000582 | >gga-miR-383 | MIMAT0003363 | >gga-miR-1659 | MIMAT0007540 |
| >rno-miR-339-5p | MIMAT0000583 | >gga-miR-455-5p | MIMAT0003364 | >gga-miR-1660 | MIMAT0007541 |
| >rno-miR-339-3p | MIMAT0004648 | >gga-miR-455-3p | MIMAT0006788 | >gga-miR-1661 | MIMAT0007542 |
| >mmu-miR-339-5p | MIMAT0000584 | >gga-miR-489 | MIMAT0003365 | >gga-miR-1662 | MIMAT0007543 |
| >mmu-miR-339-3p | MIMAT0004649 | >gga-miR-490 | MIMAT0003366 | >gga-miR-1663 | MIMAT0007545 |
| >rno-miR-340-5p | MIMAT0004650 | >gga-miR-499 | MIMAT0003367 | >gga-miR-1664 | MIMAT0007547 |
| >rno-miR-340-3p | MIMAT0000585 | >gga-miR-211 | MIMAT0003368 | >gga-miR-1665 | MIMAT0007548 |
| >mmu-miR-340-5p | MIMAT0004651 | >gga-miR-367 | MIMAT0003369 | >gga-miR-1666 | MIMAT0007549 |
| >mmu-miR-340-3p | MIMAT0000586 | >gga-miR-466 | MIMAT0003370 | >gga-miR-1667 | MIMAT0007550 |
| >rno-miR-341 | MIMAT0000587 | >gga-miR-429 | MIMAT0003371 | >gga-miR-1668 | MIMAT0007552 |
| >mmu-miR-341 | MIMAT0000588 | >gga-miR-449 | MIMAT0003372 | >gga-miR-1669 | MIMAT0007553 |
| >rno-miR-342-5p | MIMAT0004652 | >mmu-miR-302b | MIMAT0003374 | >gga-miR-1670 | MIMAT0007554 |
| >rno-miR-342-3p | MIMAT0000589 | >mmu-miR-302c | MIMAT0003376 | >gga-miR-1671 | MIMAT0007555 |
| >mmu-miR-342-5p | MIMAT0004653 | >mmu-miR-302d | MIMAT0003377 | >gga-miR-1672 | MIMAT0007556 |
| >mmu-miR-342-3p | MIMAT0000590 | >rno-miR-378 | MIMAT0003379 | >gga-miR-1673 | MIMAT0007557 |
| >rno-miR-343 | MIMAT0000591 | >rno-miR-505 | MIMAT0003380 | >gga-miR-1674 | MIMAT0007558 |
| >rno-miR-344-5p | MIMAT0004654 | >rno-miR-499 | MIMAT0003381 | >gga-miR-1675 | MIMAT0007559 |
| >rno-miR-344-3p | MIMAT0000592 | >rno-miR-664 | MIMAT0003382 | >gga-miR-1676 | MIMAT0007561 |
| >mmu-miR-344 | MIMAT0000593 | >rno-miR-497 | MIMAT0003383 | >gga-miR-1677 | MIMAT0007563 |
| >rno-miR-345-5p | MIMAT0000594 | >ebv-miR-BART3 | MIMAT0003411 | >gga-miR-1678 | MIMAT0007564 |
| >rno-miR-345-3p | MIMAT0004655 | >ebv-miR-BART4 | MIMAT0003412 | >gga-miR-1679 | MIMAT0007565 |
| >mmu-miR-345-5p | MIMAT0000595 | >ebv-miR-BART5 | MIMAT0003413 | >gga-miR-1680 | MIMAT0007567 |
| >mmu-miR-345-3p | MIMAT0004656 | >ebv-miR-BART6-5p | MIMAT0003414 | >gga-miR-1681 | MIMAT0007568 |
| >rno-miR-346 | MIMAT0000596 | >ebv-miR-BART6-3p | MIMAT0003415 | >gga-miR-1682 | MIMAT0007569 |
| >mmu-miR-346 | MIMAT0000597 | >ebv-miR-BART7 | MIMAT0003416 | >gga-miR-1683 | MIMAT0007570 |
| >rno-miR-347 | MIMAT0000598 | >ebv-miR-BART8 | MIMAT0003417 | >gga-miR-1684 | MIMAT0007572 |
| >rno-miR-349 | MIMAT0000599 | >ebv-miR-BART9 | MIMAT0003419 | >gga-miR-1685 | MIMAT0007574 |
| >rno-miR-129 | MIMAT0000600 | >ebv-miR-BART10 | MIMAT0003420 | >gga-miR-1686 | MIMAT0007575 |
| >rno-miR-20a | MIMAT0000602 | >ebv-miR-BART11-5p | MIMAT0003421 | >gga-miR-1687 | MIMAT0007576 |
| >rno-miR-350 | MIMAT0000604 | >ebv-miR-BART11-3p | MIMAT0003422 | >gga-miR-1688 | MIMAT0007578 |
| >mmu-miR-350 | MIMAT0000605 | >ebv-miR-BART12 | MIMAT0003423 | >gga-miR-1689 | MIMAT0007580 |
| >rno-miR-7a | MIMAT0000606 | >ebv-miR-BART13 | MIMAT0003424 | >gga-miR-1691 | MIMAT0007582 |
| >rno-miR-351 | MIMAT0000608 | >ebv-miR-BART14 | MIMAT0003426 | >gga-miR-199b | MIMAT0007583 |
| >mmu-miR-351 | MIMAT0000609 | >rlcv-miR-rL1-1 | MIMAT0003427 | >gga-miR-1692 | MIMAT0007584 |
| >rno-miR-352 | MIMAT0000610 | >rlcv-miR-rL1-2 | MIMAT0003428 | >gga-miR-1693 | MIMAT0007585 |
| >rno-miR-135b | MIMAT0000611 | >rlcv-miR-rL1-3 | MIMAT0003429 | >gga-miR-1694 | MIMAT0007586 |
| >mmu-miR-135b | MIMAT0000612 | >rlcv-miR-rL1-4-5p | MIMAT0003430 | >gga-miR-1695 | MIMAT0007587 |
| >rno-miR-151 | MIMAT0000613 | >rlcv-miR-rL1-4-3p | MIMAT0003431 | >gga-miR-1696 | MIMAT0007588 |
| >rno-miR-101b | MIMAT0000615 | >rlcv-miR-rL1-5-5p | MIMAT0003432 | >gga-miR-1697 | MIMAT0007589 |
| >mmu-miR-101b | MIMAT0000616 | >rlcv-miR-rL1-5-3p | MIMAT0003433 | >gga-miR-1698 | MIMAT0007590 |
| >hsa-miR-200c | MIMAT0000617 | >rlcv-miR-rL1-6-5p | MIMAT0003434 | >gga-miR-1699 | MIMAT0007591 |
| >osa-miR156a | MIMAT0000618 | >rlcv-miR-rL1-6 | MIMAT0003435 | >gga-miR-1700 | MIMAT0007592 |
| >osa-miR156b | MIMAT0000619 | >rlcv-miR-rL1-7 | MIMAT0003436 | >gga-miR-1701 | MIMAT0007593 |
| >osa-miR156c | MIMAT0000620 | >rlcv-miR-rL1-8 | MIMAT0003437 | >gga-miR-1702 | MIMAT0007594 |
| >osa-miR156d | MIMAT0000621 | >rlcv-miR-rL1-9 | MIMAT0003438 | >gga-miR-1703 | MIMAT0007595 |
| >osa-miR156e | MIMAT0000622 | >rlcv-miR-rL1-10 | MIMAT0003439 | >gga-miR-1704 | MIMAT0007596 |
| >osa-miR156f | MIMAT0000623 | >rlcv-miR-rL1-11 | MIMAT0003440 | >gga-miR-1705 | MIMAT0007597 |
| >osa-miR156g | MIMAT0000624 | >rlcv-miR-rL1-12-5p | MIMAT0003441 | >gga-miR-1706 | MIMAT0007598 |
| >osa-miR156h | MIMAT0000625 | >rlcv-miR-rL1-12-3p | MIMAT0003442 | >gga-miR-1707 | MIMAT0007599 |
| >osa-miR156i | MIMAT0000626 | >rlcv-miR-rL1-13 | MIMAT0003443 | >gga-miR-1813 | MIMAT0007600 |
| >osa-miR156j | MIMAT0000627 | >rlcv-miR-rL1-14-5p | MIMAT0003444 | >gga-miR-1708 | MIMAT0007601 |
| >osa-miR160a | MIMAT0000628 | >rlcv-miR-rL1-14-3p | MIMAT0003445 | >gga-miR-449c | MIMAT0007602 |
| >osa-miR160b | MIMAT0000629 | >rlcv-miR-rL1-15 | MIMAT0003446 | >gga-miR-1709 | MIMAT0007604 |
| >osa-miR160c | MIMAT0000630 | >rlcv-miR-rL1-16-5p | MIMAT0003447 | >gga-miR-1710 | MIMAT0007605 |
| >osa-miR160d | MIMAT0000631 | >rlcv-miR-rL1-16-3p | MIMAT0003448 | >gga-miR-1711 | MIMAT0007606 |
| >osa-miR162a | MIMAT0000632 | >hsa-miR-758 | MIMAT0003879 | >gga-miR-1712 | MIMAT0007608 |
| >osa-miR164a | MIMAT0000633 | >hsa-miR-1264 | MIMAT0005791 | >gga-miR-1713 | MIMAT0007609 |
| >osa-miR164b | MIMAT0000634 | >hsa-miR-671-5p | MIMAT0003880 | >gga-miR-1714 | MIMAT0007610 |
| >osa-miR166a | MIMAT0000635 | >hsa-miR-671-3p | MIMAT0004819 | >gga-miR-1715 | MIMAT0007611 |
| >osa-miR166b | MIMAT0000636 | >hsa-miR-668 | MIMAT0003881 | >gga-miR-1716 | MIMAT0007613 |
| >osa-miR166c | MIMAT0000637 | >hsa-miR-767-5p | MIMAT0003882 | >gga-miR-1717 | MIMAT0007614 |
| >osa-miR166d | MIMAT0000638 | >hsa-miR-767-3p | MIMAT0003883 | >gga-miR-1718 | MIMAT0007615 |
| >osa-miR166e | MIMAT0000639 | >hsa-miR-1224-5p | MIMAT0005458 | >gga-miR-1719 | MIMAT0007616 |
| >osa-miR166f | MIMAT0000640 | >hsa-miR-1224-3p | MIMAT0005459 | >gga-miR-1720 | MIMAT0007618 |
| >osa-miR167a | MIMAT0000641 | >hsa-miR-320b | MIMAT0005792 | >gga-miR-1721 | MIMAT0007619 |
| >osa-miR167b | MIMAT0000642 | >hsa-miR-320c | MIMAT0005793 | >gga-miR-1722 | MIMAT0007620 |
| >osa-miR167c | MIMAT0000643 | >hsa-miR-1296 | MIMAT0005794 | >gga-miR-1723 | MIMAT0007622 |
| >osa-miR169a | MIMAT0000644 | >hsa-miR-1468 | MIMAT0006789 | >gga-miR-1724 | MIMAT0007623 |
| >osa-miR171a | MIMAT0000645 | >hsa-miR-1323 | MIMAT0005795 | >gga-miR-1725 | MIMAT0007624 |
| >hsa-miR-155 | MIMAT0000646 | >hsa-miR-1271 | MIMAT0005796 | >gga-miR-1726 | MIMAT0007625 |
| >mmu-miR-107 | MIMAT0000647 | >hsa-miR-1301 | MIMAT0005797 | >gga-miR-1727 | MIMAT0007626 |
| >mmu-miR-10a | MIMAT0000648 | >hsa-miR-454 | MIMAT0003885 | >gga-miR-1728-5p | MIMAT0007627 |
| >mmu-miR-17 | MIMAT0000649 | >hsa-miR-1185 | MIMAT0005798 | >gga-miR-1728-3p | MIMAT0007628 |
| >mmu-miR-19a | MIMAT0000651 | >hsa-miR-1283 | MIMAT0005799 | >gga-miR-1729 | MIMAT0007629 |
| >mmu-miR-25 | MIMAT0000652 | >hsa-miR-675b | MIMAT0006790 | >gga-miR-1730 | MIMAT0007632 |
| >mmu-miR-28 | MIMAT0000653 | >hsa-miR-769-5p | MIMAT0003886 | >gga-miR-1731 | MIMAT0007633 |
| >mmu-miR-32 | MIMAT0000654 | >hsa-miR-769-3p | MIMAT0003887 | >gga-miR-1732 | MIMAT0007634 |
| >mmu-miR-100 | MIMAT0000655 | >hsa-miR-766 | MIMAT0003888 | >gga-miR-1733 | MIMAT0007635 |
| >mmu-miR-139-5p | MIMAT0000656 | >hsa-miR-802 | MIMAT0004185 | >gga-miR-1734 | MIMAT0007636 |
| >mmu-miR-139-3p | MIMAT0004662 | >hsa-miR-1298 | MIMAT0005800 | >gga-miR-1735 | MIMAT0007637 |
| >mmu-miR-200c | MIMAT0000657 | >mmu-miR-1224 | MIMAT0005460 | >gga-miR-1736 | MIMAT0007639 |
| >mmu-miR-210 | MIMAT0000658 | >mmu-miR-301b | MIMAT0004186 | >gga-miR-1737 | MIMAT0007640 |
| >mmu-miR-212 | MIMAT0000659 | >mmu-miR-675-5p | MIMAT0003725 | >gga-miR-1738 | MIMAT0007641 |
| >mmu-miR-214 | MIMAT0000661 | >mmu-miR-675-3p | MIMAT0003726 | >gga-miR-1739 | MIMAT0007642 |
| >mmu-miR-216a | MIMAT0000662 | >mmu-miR-744 | MIMAT0004187 | >gga-miR-1740 | MIMAT0007643 |
| >mmu-miR-218 | MIMAT0000663 | >mmu-miR-374 | MIMAT0003727 | >gga-miR-1741 | MIMAT0007645 |
| >mmu-miR-219 | MIMAT0000664 | >mmu-miR-216b | MIMAT0003729 | >gga-miR-1742 | MIMAT0007646 |
| >mmu-miR-223 | MIMAT0000665 | >mmu-miR-592 | MIMAT0003730 | >gga-miR-1743 | MIMAT0007647 |
| >mmu-miR-320 | MIMAT0000666 | >mmu-miR-758 | MIMAT0003889 | >gga-miR-1744 | MIMAT0007649 |
| >mmu-miR-33 | MIMAT0000667 | >mmu-miR-551b | MIMAT0003890 | >gga-miR-1745 | MIMAT0007650 |
| >mmu-miR-211 | MIMAT0000668 | >mmu-miR-671-5p | MIMAT0003731 | >gga-miR-1746 | MIMAT0007651 |
| >mmu-miR-221 | MIMAT0000669 | >mmu-miR-671-3p | MIMAT0004821 | >gga-miR-1747 | MIMAT0007652 |
| >mmu-miR-222 | MIMAT0000670 | >mmu-miR-668 | MIMAT0003732 | >gga-miR-1748 | MIMAT0007653 |
| >mmu-miR-224 | MIMAT0000671 | >mmu-miR-665 | MIMAT0003733 | >gga-miR-1749 | MIMAT0007654 |
| >mmu-miR-199b | MIMAT0004667 | >mmu-miR-667 | MIMAT0003734 | >gga-miR-1816 | MIMAT0007656 |
| >mmu-miR-181b | MIMAT0000673 | >mmu-miR-770-5p | MIMAT0004822 | >gga-miR-1750 | MIMAT0007657 |
| >mmu-miR-181c | MIMAT0000674 | >mmu-miR-770-3p | MIMAT0003891 | >gga-miR-1752 | MIMAT0007659 |
| >mmu-miR-125b-3p | MIMAT0004669 | >mmu-miR-762 | MIMAT0003892 | >gga-miR-1753 | MIMAT0007660 |
| >mmu-miR-7a | MIMAT0000677 | >mmu-miR-802 | MIMAT0004188 | >gga-miR-1754 | MIMAT0007661 |
| >mmu-miR-7b | MIMAT0000678 | >mmu-miR-672 | MIMAT0003735 | >gga-miR-1755 | MIMAT0007662 |
| >mmu-miR-217 | MIMAT0000679 | >mmu-miR-670 | MIMAT0003736 | >gga-miR-1756a | MIMAT0007663 |
| >hsa-miR-106b | MIMAT0000680 | >mmu-miR-761 | MIMAT0003893 | >gga-miR-1757 | MIMAT0007664 |
| >hsa-miR-29c | MIMAT0000681 | >mmu-miR-764-5p | MIMAT0003894 | >gga-miR-1758 | MIMAT0007665 |
| >hsa-miR-200a | MIMAT0000682 | >mmu-miR-764-3p | MIMAT0003895 | >gga-miR-1759 | MIMAT0007666 |
| >hsa-miR-302a | MIMAT0000684 | >mmu-miR-763 | MIMAT0003896 | >gga-miR-1760 | MIMAT0007667 |
| >hsa-miR-219-2-3p | MIMAT0004675 | >mmu-miR-669a | MIMAT0003477 | >gga-miR-1761 | MIMAT0007668 |
| >hsa-miR-34b | MIMAT0004676 | >mmu-miR-666-5p | MIMAT0003737 | >gga-miR-1762 | MIMAT0007669 |
| >hsa-miR-34c-5p | MIMAT0000686 | >mmu-miR-666-3p | MIMAT0004823 | >gga-miR-1763 | MIMAT0007670 |
| >hsa-miR-34c-3p | MIMAT0004677 | >mmu-miR-759 | MIMAT0003897 | >gga-miR-1764 | MIMAT0007672 |
| >hsa-miR-299-5p | MIMAT0002890 | >mmu-miR-496 | MIMAT0003738 | >gga-miR-1765 | MIMAT0007673 |
| >hsa-miR-299-3p | MIMAT0000687 | >mmu-miR-673-5p | MIMAT0003739 | >gga-miR-1766 | MIMAT0007674 |
| >hsa-miR-301a | MIMAT0000688 | >mmu-miR-673-3p | MIMAT0004824 | >gga-miR-1767 | MIMAT0007675 |
| >hsa-miR-99b | MIMAT0000689 | >mmu-miR-760 | MIMAT0003898 | >gga-miR-1768 | MIMAT0007676 |
| >hsa-miR-296-5p | MIMAT0000690 | >mmu-miR-674 | MIMAT0003740 | >gga-miR-1769 | MIMAT0007678 |
| >hsa-miR-296-3p | MIMAT0004679 | >mmu-miR-488 | MIMAT0003450 | >gga-miR-1770 | MIMAT0007679 |
| >hsa-miR-130b | MIMAT0000691 | >mmu-miR-677 | MIMAT0003451 | >gga-miR-1771 | MIMAT0007680 |
| >hsa-miR-30e | MIMAT0000692 | >mmu-miR-678 | MIMAT0003452 | >gga-miR-1772 | MIMAT0007682 |
| >cbr-miR-72 | MIMAT0000694 | >mmu-miR-497 | MIMAT0003453 | >gga-miR-1773 | MIMAT0007684 |
| >cel-miR-353 | MIMAT0000695 | >mmu-miR-423-5p | MIMAT0004825 | >gga-miR-1774 | MIMAT0007685 |
| >cel-miR-354 | MIMAT0000696 | >mmu-miR-423-3p | MIMAT0003454 | >gga-miR-1775 | MIMAT0007686 |
| >cel-miR-355 | MIMAT0000697 | >mmu-miR-679 | MIMAT0003455 | >gga-miR-1776 | MIMAT0007687 |
| >cel-miR-356 | MIMAT0000698 | >mmu-miR-495 | MIMAT0003456 | >gga-miR-1777 | MIMAT0007688 |
| >cel-miR-357 | MIMAT0000699 | >mmu-miR-680 | MIMAT0003457 | >gga-miR-1778 | MIMAT0007689 |
| >cel-miR-358 | MIMAT0000700 | >mmu-miR-681 | MIMAT0003458 | >gga-miR-1779 | MIMAT0007690 |
| >cel-miR-359 | MIMAT0000701 | >mmu-miR-682 | MIMAT0003459 | >gga-miR-1780 | MIMAT0007691 |
| >cel-miR-360 | MIMAT0000702 | >mmu-miR-449c | MIMAT0003460 | >gga-miR-1781 | MIMAT0007693 |
| >hsa-miR-361-5p | MIMAT0000703 | >mmu-miR-683 | MIMAT0003461 | >gga-miR-1782 | MIMAT0007694 |
| >hsa-miR-361-3p | MIMAT0004682 | >mmu-miR-684 | MIMAT0003462 | >gga-miR-1783 | MIMAT0007695 |
| >mmu-miR-361 | MIMAT0000704 | >mmu-miR-685 | MIMAT0003463 | >gga-miR-1785 | MIMAT0007697 |
| >hsa-miR-362-5p | MIMAT0000705 | >mmu-miR-686 | MIMAT0003464 | >gga-miR-1786 | MIMAT0007698 |
| >hsa-miR-362-3p | MIMAT0004683 | >mmu-miR-719 | MIMAT0003465 | >gga-miR-1787 | MIMAT0007699 |
| >mmu-miR-362-5p | MIMAT0000706 | >mmu-miR-687 | MIMAT0003466 | >gga-miR-1788-5p | MIMAT0007700 |
| >mmu-miR-362-3p | MIMAT0004684 | >mmu-miR-688 | MIMAT0003467 | >gga-miR-1788-3p | MIMAT0007701 |
| >hsa-miR-363 | MIMAT0000707 | >mmu-miR-689 | MIMAT0003468 | >gga-miR-1789 | MIMAT0007702 |
| >mmu-miR-363 | MIMAT0000708 | >mmu-miR-690 | MIMAT0003469 | >gga-miR-1790 | MIMAT0007703 |
| >hsa-miR-365 | MIMAT0000710 | >mmu-miR-691 | MIMAT0003470 | >gga-miR-1791 | MIMAT0007705 |
| >mmu-miR-365 | MIMAT0000711 | >mmu-miR-692 | MIMAT0003471 | >gga-miR-1792 | MIMAT0007706 |
| >hsa-miR-302b | MIMAT0000715 | >mmu-miR-693-5p | MIMAT0003472 | >gga-miR-1793 | MIMAT0007707 |
| >hsa-miR-302c | MIMAT0000717 | >mmu-miR-693-3p | MIMAT0004189 | >gga-miR-1794 | MIMAT0007708 |
| >hsa-miR-302d | MIMAT0000718 | >mmu-miR-694 | MIMAT0003474 | >gga-miR-1795 | MIMAT0007709 |
| >hsa-miR-367 | MIMAT0000719 | >mmu-miR-146b | MIMAT0003475 | >gga-miR-1796 | MIMAT0007710 |
| >hsa-miR-376c | MIMAT0000720 | >mmu-miR-669b | MIMAT0003476 | >gga-miR-1797 | MIMAT0007711 |
| >hsa-miR-369-5p | MIMAT0001621 | >mmu-miR-467b | MIMAT0005448 | >gga-miR-1756b | MIMAT0007712 |
| >hsa-miR-369-3p | MIMAT0000721 | >mmu-miR-669c | MIMAT0003479 | >gga-miR-1798 | MIMAT0007714 |
| >hsa-miR-370 | MIMAT0000722 | >mmu-miR-297b-5p | MIMAT0003480 | >gga-miR-1799 | MIMAT0007715 |
| >hsa-miR-371-5p | MIMAT0004687 | >mmu-miR-297b-3p | MIMAT0004827 | >gga-miR-1800 | MIMAT0007716 |
| >hsa-miR-371-3p | MIMAT0000723 | >mmu-miR-695 | MIMAT0003481 | >gga-miR-1801 | MIMAT0007717 |
| >hsa-miR-372 | MIMAT0000724 | >mmu-miR-499 | MIMAT0003482 | >gga-miR-1802 | MIMAT0007718 |
| >hsa-miR-373 | MIMAT0000726 | >mmu-miR-696 | MIMAT0003483 | >gga-miR-122b | MIMAT0007719 |
| >hsa-miR-374a | MIMAT0000727 | >mmu-miR-720 | MIMAT0003484 | >gga-miR-1803 | MIMAT0007720 |
| >hsa-miR-375 | MIMAT0000728 | >mmu-miR-455 | MIMAT0003742 | >gga-miR-1804 | MIMAT0007721 |
| >hsa-miR-376a | MIMAT0000729 | >mmu-miR-491 | MIMAT0003486 | >gga-miR-1805-5p | MIMAT0007722 |
| >hsa-miR-377 | MIMAT0000730 | >mmu-miR-697 | MIMAT0003487 | >gga-miR-1805-3p | MIMAT0007723 |
| >hsa-miR-378 | MIMAT0000732 | >mmu-miR-698 | MIMAT0003488 | >gga-miR-1806 | MIMAT0007724 |
| >hsa-miR-379 | MIMAT0000733 | >mmu-miR-699 | MIMAT0003489 | >gga-miR-1807 | MIMAT0007725 |
| >hsa-miR-380 | MIMAT0000735 | >mmu-miR-700 | MIMAT0003490 | >gga-miR-1808 | MIMAT0007726 |
| >hsa-miR-381 | MIMAT0000736 | >mmu-miR-701 | MIMAT0003491 | >gga-miR-1809 | MIMAT0007727 |
| >hsa-miR-382 | MIMAT0000737 | >mmu-miR-702 | MIMAT0003492 | >gga-miR-1810 | MIMAT0007728 |
| >hsa-miR-383 | MIMAT0000738 | >mmu-miR-703 | MIMAT0003493 | >gga-miR-1811 | MIMAT0007729 |
| >mmu-miR-375 | MIMAT0000739 | >mmu-miR-704 | MIMAT0003494 | >gga-miR-1812 | MIMAT0007730 |
| >mmu-miR-376a | MIMAT0000740 | >mmu-miR-705 | MIMAT0003495 | >gga-miR-10a | MIMAT0007731 |
| >mmu-miR-377 | MIMAT0000741 | >mmu-miR-706 | MIMAT0003496 | >gga-miR-130c | MIMAT0007734 |
| >mmu-miR-378 | MIMAT0003151 | >mmu-miR-707 | MIMAT0003497 | >gga-miR-146c | MIMAT0007735 |
| >mmu-miR-379 | MIMAT0000743 | >mmu-miR-708 | MIMAT0004828 | >gga-miR-15c | MIMAT0007737 |
| >mmu-miR-380-5p | MIMAT0000744 | >mmu-miR-709 | MIMAT0003499 | >gga-miR-16c | MIMAT0007739 |
| >mmu-miR-380-3p | MIMAT0000745 | >mmu-miR-710 | MIMAT0003500 | >gga-miR-193a | MIMAT0007740 |
| >mmu-miR-381 | MIMAT0000746 | >mmu-miR-711 | MIMAT0003501 | >gga-miR-301b-5p | MIMAT0007741 |
| >mmu-miR-382 | MIMAT0000747 | >mmu-miR-712 | MIMAT0003502 | >gga-miR-301b-3p | MIMAT0007742 |
| >mmu-miR-383 | MIMAT0000748 | >mmu-miR-713 | MIMAT0003504 | >gga-miR-449b | MIMAT0007743 |
| >cel-lsy-6 | MIMAT0000749 | >mmu-miR-714 | MIMAT0003505 | >gga-miR-458 | MIMAT0007746 |
| >hsa-miR-340 | MIMAT0004692 | >mmu-miR-715 | MIMAT0003506 | >mml-miR-1 | MIMAT0006150 |
| >hsa-miR-330-5p | MIMAT0004693 | >mmu-miR-500 | MIMAT0003507 | >mml-let-7a | MIMAT0006151 |
| >hsa-miR-330-3p | MIMAT0000751 | >mmu-miR-501-5p | MIMAT0003508 | >mml-let-7b | MIMAT0006152 |
| >hsa-miR-328 | MIMAT0000752 | >mmu-miR-501-3p | MIMAT0003509 | >mml-let-7c | MIMAT0006153 |
| >hsa-miR-342-5p | MIMAT0004694 | >mmu-miR-717 | MIMAT0003510 | >mml-let-7d | MIMAT0006154 |
| >hsa-miR-342-3p | MIMAT0000753 | >mmu-miR-450b-5p | MIMAT0003511 | >mml-let-7e | MIMAT0006155 |
| >hsa-miR-337-5p | MIMAT0004695 | >mmu-miR-450b-3p | MIMAT0003512 | >mml-let-7f | MIMAT0006156 |
| >hsa-miR-337-3p | MIMAT0000754 | >mmu-miR-505 | MIMAT0003513 | >mml-let-7g | MIMAT0006157 |
| >hsa-miR-323-5p | MIMAT0004696 | >mmu-miR-718 | MIMAT0003514 | >mml-let-7i | MIMAT0006158 |
| >hsa-miR-323-3p | MIMAT0000755 | >mmu-miR-721 | MIMAT0003515 | >mml-miR-7 | MIMAT0006159 |
| >hsa-miR-326 | MIMAT0000756 | >ppt-miR1211 | MIMAT0004829 | >mml-miR-9 | MIMAT0006160 |
| >hsa-miR-151-5p | MIMAT0004697 | >ppt-miR1212 | MIMAT0003901 | >mml-miR-10a | MIMAT0006161 |
| >hsa-miR-151-3p | MIMAT0000757 | >ppt-miR1214 | MIMAT0003903 | >mml-miR-10b | MIMAT0006162 |
| >hsa-miR-135b | MIMAT0000758 | >ppt-miR1215 | MIMAT0003904 | >mml-miR-18b | MIMAT0006163 |
| >hsa-miR-148b | MIMAT0000759 | >ppt-miR1216 | MIMAT0003905 | >mml-miR-20b | MIMAT0006164 |
| >hsa-miR-331-5p | MIMAT0004700 | >ppt-miR1217-5p | MIMAT0004830 | >mml-miR-23b | MIMAT0006165 |
| >hsa-miR-331-3p | MIMAT0000760 | >ppt-miR1217-3p | MIMAT0003906 | >mml-miR-26b | MIMAT0006166 |
| >hsa-miR-324-5p | MIMAT0000761 | >ppt-miR1218 | MIMAT0003907 | >mml-miR-27b | MIMAT0006167 |
| >hsa-miR-324-3p | MIMAT0000762 | >ppt-miR1219a | MIMAT0003908 | >mml-miR-29b | MIMAT0006168 |
| >hsa-miR-338-5p | MIMAT0004701 | >ppt-miR1219b | MIMAT0003909 | >mml-miR-29c | MIMAT0006169 |
| >hsa-miR-338-3p | MIMAT0000763 | >ppt-miR1219c | MIMAT0003910 | >mml-miR-30c | MIMAT0006170 |
| >hsa-miR-339-5p | MIMAT0000764 | >ppt-miR1219d | MIMAT0003911 | >mml-miR-30d | MIMAT0006171 |
| >hsa-miR-339-3p | MIMAT0004702 | >ppt-miR1220a | MIMAT0004831 | >mml-miR-30e | MIMAT0006172 |
| >hsa-miR-335 | MIMAT0000765 | >ppt-miR1220b | MIMAT0004832 | >mml-miR-33b | MIMAT0006173 |
| >mmu-miR-335-5p | MIMAT0000766 | >ppt-miR533b-5p | MIMAT0003914 | >mml-miR-34b | MIMAT0006174 |
| >mmu-miR-335-3p | MIMAT0004704 | >ppt-miR533b-3p | MIMAT0004833 | >mml-miR-34c-5p | MIMAT0006175 |
| >cbr-lsy-6 | MIMAT0000767 | >ppt-miR535d | MIMAT0003915 | >mml-miR-34c-3p | MIMAT0006176 |
| >cel-miR-392 | MIMAT0000768 | >ppt-miR1221-5p | MIMAT0003916 | >mml-miR-92b | MIMAT0006177 |
| >mmu-miR-133b | MIMAT0000769 | >ppt-miR1221-3p | MIMAT0004834 | >mml-miR-95 | MIMAT0006178 |
| >hsa-miR-133b | MIMAT0000770 | >ppt-miR1222a | MIMAT0003917 | >mml-miR-99b | MIMAT0006179 |
| >hsa-miR-325 | MIMAT0000771 | >ppt-miR1223a | MIMAT0003918 | >mml-miR-122a | MIMAT0006180 |
| >hsa-miR-345 | MIMAT0000772 | >ppt-miR390c | MIMAT0003919 | >mml-miR-125a-5p | MIMAT0006181 |
| >hsa-miR-346 | MIMAT0000773 | >hsv1-miR-H1 | MIMAT0003744 | >mml-miR-125a-3p | MIMAT0006182 |
| >rno-let-7a | MIMAT0000774 | >bta-miR-26a | MIMAT0003516 | >mml-miR-126 | MIMAT0006183 |
| >rno-let-7b | MIMAT0000775 | >bta-miR-18b | MIMAT0003517 | >mml-miR-128b | MIMAT0006184 |
| >rno-let-7c | MIMAT0000776 | >bta-miR-29a | MIMAT0003518 | >mml-miR-129-5p | MIMAT0006185 |
| >rno-let-7e | MIMAT0000777 | >bta-let-7f | MIMAT0003519 | >mml-miR-129-3p | MIMAT0006186 |
| >rno-let-7f | MIMAT0000778 | >bta-miR-101 | MIMAT0003520 | >mml-miR-130b | MIMAT0006187 |
| >rno-let-7i | MIMAT0000779 | >bta-miR-103 | MIMAT0003521 | >mml-miR-132 | MIMAT0006188 |
| >rno-miR-7b | MIMAT0000780 | >bta-miR-148a | MIMAT0003522 | >mml-miR-133c | MIMAT0006777 |
| >rno-miR-9 | MIMAT0000781 | >bta-miR-151 | MIMAT0003524 | >mml-miR-133b | MIMAT0006189 |
| >rno-miR-10a-5p | MIMAT0000782 | >bta-miR-16b | MIMAT0003525 | >mml-miR-134 | MIMAT0006190 |
| >rno-miR-10a-3p | MIMAT0004709 | >bta-miR-18a | MIMAT0003526 | >mml-miR-135b | MIMAT0006191 |
| >rno-miR-10b | MIMAT0000783 | >bta-miR-20a | MIMAT0003527 | >mml-miR-136 | MIMAT0006192 |
| >rno-miR-15b | MIMAT0000784 | >bta-miR-21 | MIMAT0003528 | >mml-miR-137 | MIMAT0006193 |
| >rno-miR-16 | MIMAT0000785 | >bta-miR-221 | MIMAT0003529 | >mml-miR-138 | MIMAT0006194 |
| >rno-miR-17-5p | MIMAT0000786 | >bta-miR-222 | MIMAT0003530 | >mml-miR-139-5p | MIMAT0006195 |
| >rno-miR-17-3p | MIMAT0004710 | >bta-miR-26b | MIMAT0003531 | >mml-miR-139-3p | MIMAT0006196 |
| >rno-miR-18a | MIMAT0000787 | >bta-miR-27a | MIMAT0003532 | >mml-miR-140-5p | MIMAT0006197 |
| >rno-miR-19b | MIMAT0000788 | >bta-miR-30d | MIMAT0003533 | >mml-miR-140-3p | MIMAT0006198 |
| >rno-miR-19a | MIMAT0000789 | >bta-miR-320 | MIMAT0003534 | >mml-miR-142-5p | MIMAT0006199 |
| >rno-miR-21 | MIMAT0000790 | >bta-miR-484 | MIMAT0003535 | >mml-miR-142-3p | MIMAT0006200 |
| >rno-miR-22 | MIMAT0000791 | >bta-miR-499 | MIMAT0003536 | >mml-miR-143 | MIMAT0006201 |
| >rno-miR-23a | MIMAT0000792 | >bta-miR-99a | MIMAT0003537 | >mml-miR-144 | MIMAT0006202 |
| >rno-miR-23b | MIMAT0000793 | >bta-miR-125a | MIMAT0003538 | >mml-miR-146a | MIMAT0006203 |
| >rno-miR-24 | MIMAT0000794 | >bta-miR-125b | MIMAT0003539 | >mml-miR-146b-5p | MIMAT0006204 |
| >rno-miR-25 | MIMAT0000795 | >bta-miR-126 | MIMAT0003540 | >mml-miR-146b-3p | MIMAT0006205 |
| >rno-miR-26a | MIMAT0000796 | >bta-miR-128 | MIMAT0003541 | >mml-miR-147a | MIMAT0006206 |
| >rno-miR-26b | MIMAT0000797 | >bta-miR-145 | MIMAT0003542 | >mml-miR-147b | MIMAT0006207 |
| >rno-miR-27b | MIMAT0000798 | >bta-miR-181a | MIMAT0003543 | >mml-miR-148a | MIMAT0006208 |
| >rno-miR-27a | MIMAT0000799 | >bta-miR-199a-5p | MIMAT0003544 | >mml-miR-148b | MIMAT0006209 |
| >rno-miR-28 | MIMAT0000800 | >bta-miR-199a-3p | MIMAT0003746 | >mml-miR-149 | MIMAT0006210 |
| >rno-miR-29b | MIMAT0000801 | >bta-miR-205 | MIMAT0003545 | >mml-miR-150 | MIMAT0006211 |
| >rno-miR-29a | MIMAT0000802 | >bta-miR-27b | MIMAT0003546 | >mml-miR-151-5p | MIMAT0006212 |
| >rno-miR-29c | MIMAT0000803 | >bta-miR-30b | MIMAT0003547 | >mml-miR-151-3p | MIMAT0006213 |
| >rno-miR-30c | MIMAT0000804 | >bta-miR-31 | MIMAT0003548 | >mml-miR-152 | MIMAT0006214 |
| >rno-miR-30e | MIMAT0000805 | >bta-miR-34b | MIMAT0003549 | >mml-miR-154 | MIMAT0006215 |
| >rno-miR-30b-5p | MIMAT0000806 | >dre-miR-190b | MIMAT0003747 | >mml-miR-155 | MIMAT0006216 |
| >rno-miR-30b-3p | MIMAT0004721 | >dre-miR-722 | MIMAT0003748 | >mml-miR-181d | MIMAT0006217 |
| >rno-miR-30d | MIMAT0000807 | >dre-miR-499 | MIMAT0003749 | >mml-miR-184 | MIMAT0006218 |
| >rno-miR-30a | MIMAT0000808 | >dre-miR-723 | MIMAT0003751 | >mml-miR-185 | MIMAT0006219 |
| >rno-miR-31 | MIMAT0000810 | >dre-miR-724 | MIMAT0003752 | >mml-miR-186 | MIMAT0006220 |
| >rno-miR-32 | MIMAT0000811 | >dre-miR-725 | MIMAT0003753 | >mml-miR-187 | MIMAT0006221 |
| >rno-miR-33 | MIMAT0000812 | >dre-miR-726 | MIMAT0003754 | >mml-miR-190b | MIMAT0006222 |
| >rno-miR-34b | MIMAT0000813 | >dre-miR-727 | MIMAT0003756 | >mml-miR-191 | MIMAT0006223 |
| >rno-miR-34c | MIMAT0000814 | >dre-miR-728 | MIMAT0003757 | >mml-miR-192 | MIMAT0006224 |
| >rno-miR-34a | MIMAT0000815 | >dre-miR-729 | MIMAT0003758 | >mml-miR-193a-5p | MIMAT0006225 |
| >rno-miR-92a | MIMAT0000816 | >dre-miR-34c | MIMAT0003759 | >mml-miR-193a-3p | MIMAT0006226 |
| >rno-miR-93 | MIMAT0000817 | >dre-miR-730 | MIMAT0003760 | >mml-miR-193b | MIMAT0006227 |
| >rno-miR-96 | MIMAT0000818 | >dre-miR-731 | MIMAT0003761 | >mml-miR-195 | MIMAT0006228 |
| >rno-miR-98 | MIMAT0000819 | >dre-miR-732 | MIMAT0003762 | >mml-miR-196b | MIMAT0006229 |
| >rno-miR-99a | MIMAT0000820 | >dre-miR-733 | MIMAT0003763 | >mml-miR-197 | MIMAT0006230 |
| >rno-miR-99b | MIMAT0000821 | >dre-miR-15c | MIMAT0003764 | >mml-miR-199a-5p | MIMAT0006231 |
| >rno-miR-100 | MIMAT0000822 | >dre-miR-734 | MIMAT0003765 | >mml-miR-199a-3p | MIMAT0006232 |
| >rno-miR-101a | MIMAT0000823 | >dre-miR-735 | MIMAT0003766 | >mml-miR-200a | MIMAT0006233 |
| >rno-miR-103 | MIMAT0000824 | >dre-miR-736 | MIMAT0003767 | >mml-miR-203 | MIMAT0006234 |
| >rno-miR-106b | MIMAT0000825 | >dre-miR-737 | MIMAT0003768 | >mml-miR-204 | MIMAT0006235 |
| >rno-miR-107 | MIMAT0000826 | >dre-miR-738 | MIMAT0003769 | >mml-miR-205 | MIMAT0006236 |
| >rno-miR-122 | MIMAT0000827 | >dre-miR-739 | MIMAT0003770 | >mml-miR-206 | MIMAT0006237 |
| >rno-miR-124 | MIMAT0000828 | >dre-miR-740 | MIMAT0003771 | >mml-miR-208a | MIMAT0006238 |
| >rno-miR-125a-5p | MIMAT0000829 | >xtr-let-7b | MIMAT0003550 | >mml-miR-208b | MIMAT0006239 |
| >rno-miR-125a-3p | MIMAT0004729 | >xtr-miR-1a | MIMAT0003551 | >mml-miR-210 | MIMAT0006240 |
| >rno-miR-125b-5p | MIMAT0000830 | >xtr-miR-7 | MIMAT0003552 | >mml-miR-212 | MIMAT0006241 |
| >rno-miR-125b-3p | MIMAT0004730 | >xtr-miR-9a | MIMAT0003553 | >mml-miR-216a | MIMAT0006242 |
| >rno-miR-126 | MIMAT0000832 | >xtr-miR-9b | MIMAT0003555 | >mml-miR-216b | MIMAT0006243 |
| >rno-miR-127 | MIMAT0000833 | >xtr-miR-10a | MIMAT0003557 | >mml-miR-217 | MIMAT0006244 |
| >rno-miR-128 | MIMAT0000834 | >xtr-miR-10b | MIMAT0003558 | >mml-miR-219-5p | MIMAT0006245 |
| >rno-miR-130a | MIMAT0000836 | >xtr-miR-10c | MIMAT0003559 | >mml-miR-219-3p | MIMAT0006246 |
| >rno-miR-130b | MIMAT0000837 | >xtr-miR-15a | MIMAT0003560 | >mml-miR-220b | MIMAT0006247 |
| >rno-miR-132 | MIMAT0000838 | >xtr-miR-15b | MIMAT0003561 | >mml-miR-220c | MIMAT0006248 |
| >rno-miR-133a | MIMAT0000839 | >xtr-miR-16c | MIMAT0003562 | >mml-miR-220d | MIMAT0006249 |
| >rno-miR-134 | MIMAT0000840 | >xtr-miR-16a | MIMAT0003563 | >mml-miR-222 | MIMAT0006250 |
| >rno-miR-135a | MIMAT0000841 | >xtr-miR-17-5p | MIMAT0003564 | >mml-miR-296-5p | MIMAT0006251 |
| >rno-miR-136 | MIMAT0000842 | >xtr-miR-17-3p | MIMAT0003565 | >mml-miR-296-3p | MIMAT0006252 |
| >rno-miR-137 | MIMAT0000843 | >xtr-miR-19a | MIMAT0003566 | >mml-miR-297 | MIMAT0006253 |
| >rno-miR-138 | MIMAT0000844 | >xtr-miR-19b | MIMAT0003567 | >mml-miR-298 | MIMAT0006254 |
| >rno-miR-139-5p | MIMAT0000845 | >xtr-miR-23a | MIMAT0003568 | >mml-miR-299-5p | MIMAT0006255 |
| >rno-miR-139-3p | MIMAT0004735 | >xtr-miR-26 | MIMAT0003569 | >mml-miR-299-3p | MIMAT0006256 |
| >rno-miR-141 | MIMAT0000846 | >xtr-miR-27a | MIMAT0003570 | >mml-miR-301a | MIMAT0006257 |
| >rno-miR-142-5p | MIMAT0000847 | >xtr-miR-27b | MIMAT0003571 | >mml-miR-301b | MIMAT0006258 |
| >rno-miR-142-3p | MIMAT0000848 | >xtr-miR-29d | MIMAT0003572 | >mml-miR-302a | MIMAT0006259 |
| >rno-miR-143 | MIMAT0000849 | >xtr-miR-29b | MIMAT0003573 | >mml-miR-302b | MIMAT0006260 |
| >rno-miR-144 | MIMAT0000850 | >xtr-miR-30a-5p | MIMAT0003574 | >mml-miR-302c | MIMAT0006261 |
| >rno-miR-145 | MIMAT0000851 | >xtr-miR-30a-3p | MIMAT0003575 | >mml-miR-302d | MIMAT0006262 |
| >rno-miR-146a | MIMAT0000852 | >xtr-miR-30b | MIMAT0003576 | >mml-miR-320 | MIMAT0006263 |
| >rno-miR-150 | MIMAT0000853 | >xtr-miR-30c | MIMAT0003577 | >mml-miR-323-5p | MIMAT0006264 |
| >rno-miR-152 | MIMAT0000854 | >xtr-miR-34a | MIMAT0003578 | >mml-miR-323-3p | MIMAT0006265 |
| >rno-miR-153 | MIMAT0000855 | >xtr-miR-34b | MIMAT0003579 | >mml-miR-324-5p | MIMAT0006266 |
| >rno-miR-154 | MIMAT0000856 | >xtr-miR-92a | MIMAT0003580 | >mml-miR-324-3p | MIMAT0006267 |
| >rno-miR-181c | MIMAT0000857 | >xtr-miR-98 | MIMAT0003581 | >mml-miR-325 | MIMAT0006268 |
| >rno-miR-181a | MIMAT0000858 | >xtr-miR-99 | MIMAT0003582 | >mml-miR-329 | MIMAT0006269 |
| >rno-miR-181b | MIMAT0000859 | >xtr-miR-106 | MIMAT0003583 | >mml-miR-330-5p | MIMAT0006270 |
| >rno-miR-183 | MIMAT0000860 | >xtr-miR-107 | MIMAT0003584 | >mml-miR-330-3p | MIMAT0006271 |
| >rno-miR-184 | MIMAT0000861 | >xtr-miR-122 | MIMAT0003585 | >mml-miR-331-5p | MIMAT0006272 |
| >rno-miR-185 | MIMAT0000862 | >xtr-miR-125b | MIMAT0003586 | >mml-miR-331-3p | MIMAT0006273 |
| >rno-miR-186 | MIMAT0000863 | >xtr-miR-126 | MIMAT0003588 | >mml-miR-335 | MIMAT0006274 |
| >rno-miR-187 | MIMAT0000864 | >xtr-miR-128 | MIMAT0003589 | >mml-miR-337-5p | MIMAT0006275 |
| >rno-miR-190 | MIMAT0000865 | >xtr-miR-129 | MIMAT0003590 | >mml-miR-337-3p | MIMAT0006276 |
| >rno-miR-191 | MIMAT0000866 | >xtr-miR-130a | MIMAT0003591 | >mml-miR-338-5p | MIMAT0006277 |
| >rno-miR-192 | MIMAT0000867 | >xtr-miR-130c | MIMAT0003592 | >mml-miR-338-3p | MIMAT0006278 |
| >rno-miR-193 | MIMAT0000868 | >xtr-miR-130b | MIMAT0003593 | >mml-miR-339-5p | MIMAT0006279 |
| >rno-miR-194 | MIMAT0000869 | >xtr-miR-132 | MIMAT0003594 | >mml-miR-339-3p | MIMAT0006280 |
| >rno-miR-195 | MIMAT0000870 | >xtr-miR-133c | MIMAT0003595 | >mml-miR-340 | MIMAT0006281 |
| >rno-miR-196a | MIMAT0000871 | >xtr-miR-133d | MIMAT0003596 | >mml-miR-342-5p | MIMAT0006282 |
| >rno-miR-199a-5p | MIMAT0000872 | >xtr-miR-133b | MIMAT0003597 | >mml-miR-342-3p | MIMAT0006283 |
| >rno-miR-199a-3p | MIMAT0004738 | >xtr-miR-135 | MIMAT0003598 | >mml-miR-345 | MIMAT0006284 |
| >rno-miR-200c | MIMAT0000873 | >xtr-miR-138 | MIMAT0003599 | >mml-miR-346 | MIMAT0006285 |
| >rno-miR-200a | MIMAT0000874 | >xtr-miR-139 | MIMAT0003600 | >mml-miR-361-5p | MIMAT0006286 |
| >rno-miR-200b | MIMAT0000875 | >xtr-miR-140 | MIMAT0003601 | >mml-miR-361-3p | MIMAT0006287 |
| >rno-miR-203 | MIMAT0000876 | >xtr-miR-142-5p | MIMAT0003602 | >mml-miR-362-5p | MIMAT0006288 |
| >rno-miR-204 | MIMAT0000877 | >xtr-miR-142-3p | MIMAT0003603 | >mml-miR-362-3p | MIMAT0006289 |
| >rno-miR-205 | MIMAT0000878 | >xtr-miR-146 | MIMAT0003604 | >mml-miR-363 | MIMAT0006290 |
| >rno-miR-206 | MIMAT0000879 | >xtr-miR-148b | MIMAT0003605 | >mml-miR-365 | MIMAT0006291 |
| >rno-miR-208 | MIMAT0000880 | >xtr-miR-150 | MIMAT0003606 | >mml-miR-367 | MIMAT0006292 |
| >rno-miR-210 | MIMAT0000881 | >xtr-miR-153 | MIMAT0003607 | >mml-miR-369-5p | MIMAT0006293 |
| >rno-miR-211 | MIMAT0000882 | >xtr-miR-155 | MIMAT0003608 | >mml-miR-369-3p | MIMAT0006294 |
| >rno-miR-212 | MIMAT0000883 | >xtr-miR-181b | MIMAT0003609 | >mml-miR-370 | MIMAT0006295 |
| >rno-miR-214 | MIMAT0000885 | >xtr-miR-182 | MIMAT0003610 | >mml-miR-371-5p | MIMAT0006296 |
| >rno-miR-216a | MIMAT0000886 | >xtr-miR-183 | MIMAT0003612 | >mml-miR-371-3p | MIMAT0006297 |
| >rno-miR-217 | MIMAT0000887 | >xtr-miR-184 | MIMAT0003613 | >mml-miR-372 | MIMAT0006298 |
| >rno-miR-218 | MIMAT0000888 | >xtr-miR-187 | MIMAT0003614 | >mml-miR-373 | MIMAT0006299 |
| >rno-miR-219-5p | MIMAT0000889 | >xtr-miR-192 | MIMAT0003615 | >mml-miR-374a | MIMAT0006300 |
| >rno-miR-219-1-3p | MIMAT0004741 | >xtr-miR-193 | MIMAT0003616 | >mml-miR-374b | MIMAT0006301 |
| >rno-miR-219-2-3p | MIMAT0005446 | >xtr-miR-194 | MIMAT0003617 | >mml-miR-375 | MIMAT0006302 |
| >rno-miR-221 | MIMAT0000890 | >xtr-miR-199a | MIMAT0003618 | >mml-miR-376a | MIMAT0006303 |
| >rno-miR-222 | MIMAT0000891 | >xtr-miR-202 | MIMAT0003621 | >mml-miR-376b | MIMAT0006304 |
| >rno-miR-223 | MIMAT0000892 | >xtr-miR-205b | MIMAT0003622 | >mml-miR-376c | MIMAT0006305 |
| >rno-miR-290 | MIMAT0000893 | >xtr-miR-206 | MIMAT0003623 | >mml-miR-377 | MIMAT0006306 |
| >rno-miR-291a-5p | MIMAT0000894 | >xtr-miR-210 | MIMAT0003624 | >mml-miR-378 | MIMAT0006307 |
| >rno-miR-291a-3p | MIMAT0000895 | >xtr-miR-181a | MIMAT0003625 | >mml-miR-379 | MIMAT0006308 |
| >rno-miR-292-5p | MIMAT0000896 | >xtr-miR-214 | MIMAT0003627 | >mml-miR-380 | MIMAT0006309 |
| >rno-miR-292-3p | MIMAT0000897 | >xtr-miR-215 | MIMAT0003628 | >mml-miR-381 | MIMAT0006310 |
| >rno-miR-296 | MIMAT0004742 | >xtr-miR-216 | MIMAT0003629 | >mml-miR-382 | MIMAT0006311 |
| >rno-miR-297 | MIMAT0000899 | >xtr-miR-217 | MIMAT0003630 | >mml-miR-383 | MIMAT0006312 |
| >rno-miR-298 | MIMAT0000900 | >xtr-miR-218 | MIMAT0003631 | >mml-miR-384 | MIMAT0006313 |
| >rno-miR-299 | MIMAT0000901 | >xtr-miR-219 | MIMAT0003632 | >mml-miR-409-5p | MIMAT0006314 |
| >rno-miR-300-5p | MIMAT0004743 | >xtr-miR-222 | MIMAT0003633 | >mml-miR-409-3p | MIMAT0006315 |
| >rno-miR-300-3p | MIMAT0000902 | >xtr-miR-223 | MIMAT0003634 | >mml-miR-410 | MIMAT0006316 |
| >rno-miR-320 | MIMAT0000903 | >xtr-miR-301 | MIMAT0003635 | >mml-miR-411 | MIMAT0006317 |
| >mmu-miR-215 | MIMAT0000904 | >xtr-miR-302 | MIMAT0003636 | >mml-miR-412 | MIMAT0006318 |
| >ath-miR167d | MIMAT0000905 | >xtr-miR-365 | MIMAT0003637 | >mml-miR-421 | MIMAT0006319 |
| >ath-miR169b | MIMAT0000906 | >xtr-miR-367 | MIMAT0003638 | >mml-miR-422a | MIMAT0006320 |
| >ath-miR169c | MIMAT0000907 | >xtr-miR-383 | MIMAT0003639 | >mml-miR-423-5p | MIMAT0006321 |
| >ath-miR169d | MIMAT0000908 | >xtr-miR-425-5p | MIMAT0003640 | >mml-miR-423-3p | MIMAT0006322 |
| >ath-miR169e | MIMAT0000909 | >xtr-miR-455 | MIMAT0003641 | >mml-miR-424 | MIMAT0006323 |
| >ath-miR169f | MIMAT0000910 | >xtr-miR-489 | MIMAT0003642 | >mml-miR-425 | MIMAT0006324 |
| >ath-miR169g | MIMAT0000911 | >xtr-miR-499 | MIMAT0003643 | >mml-miR-429 | MIMAT0006325 |
| >ath-miR169h | MIMAT0000913 | >xtr-let-7c | MIMAT0003644 | >mml-miR-431 | MIMAT0006326 |
| >ath-miR169i | MIMAT0000914 | >xtr-let-7f | MIMAT0003645 | >mml-miR-432 | MIMAT0006327 |
| >ath-miR169j | MIMAT0000915 | >xtr-let-7g | MIMAT0003646 | >mml-miR-433 | MIMAT0006328 |
| >ath-miR169k | MIMAT0000916 | >xtr-let-7i | MIMAT0003647 | >mml-miR-448 | MIMAT0006329 |
| >ath-miR169l | MIMAT0000917 | >xtr-miR-1b | MIMAT0003648 | >mml-miR-449a | MIMAT0006330 |
| >ath-miR169m | MIMAT0000918 | >xtr-miR-9 | MIMAT0003649 | >mml-miR-449b | MIMAT0006331 |
| >ath-miR169n | MIMAT0000919 | >xtr-miR-15c | MIMAT0003651 | >mml-miR-450a | MIMAT0006332 |
| >ath-miR171b | MIMAT0000920 | >xtr-miR-18a | MIMAT0003652 | >mml-miR-450b-5p | MIMAT0006333 |
| >ath-miR171c | MIMAT0000921 | >xtr-miR-189 | MIMAT0003653 | >mml-miR-450b-3p | MIMAT0006334 |
| >ath-miR172c | MIMAT0000922 | >xtr-miR-24a | MIMAT0003654 | >mml-miR-451 | MIMAT0006335 |
| >ath-miR172d | MIMAT0000923 | >xtr-miR-24b | MIMAT0003655 | >mml-miR-452 | MIMAT0006336 |
| >ath-miR390a | MIMAT0000931 | >xtr-miR-29a | MIMAT0003656 | >mml-miR-453 | MIMAT0006337 |
| >ath-miR390b | MIMAT0000932 | >xtr-miR-30d | MIMAT0003657 | >mml-miR-454 | MIMAT0006338 |
| >ath-miR391 | MIMAT0000933 | >xtr-miR-92b | MIMAT0003658 | >mml-miR-455-5p | MIMAT0006339 |
| >ath-miR393a | MIMAT0000934 | >xtr-miR-93a | MIMAT0003659 | >mml-miR-455-3p | MIMAT0006340 |
| >ath-miR393b | MIMAT0000935 | >xtr-miR-93b | MIMAT0003660 | >mml-miR-484 | MIMAT0006341 |
| >ath-miR394a | MIMAT0000936 | >xtr-miR-96 | MIMAT0003661 | >mml-miR-485-5p | MIMAT0006342 |
| >ath-miR394b | MIMAT0000937 | >xtr-miR-101a | MIMAT0003662 | >mml-miR-485-3p | MIMAT0006343 |
| >ath-miR395a | MIMAT0000938 | >xtr-miR-103 | MIMAT0003663 | >mml-miR-486-5p | MIMAT0006344 |
| >ath-miR395b | MIMAT0000939 | >xtr-miR-148a | MIMAT0003664 | >mml-miR-486-3p | MIMAT0006345 |
| >ath-miR395c | MIMAT0000940 | >xtr-miR-338 | MIMAT0003665 | >mml-miR-487a | MIMAT0006346 |
| >ath-miR395d | MIMAT0000941 | >xtr-let-7e | MIMAT0003666 | >mml-miR-487b | MIMAT0006347 |
| >ath-miR395e | MIMAT0000942 | >xtr-let-7a | MIMAT0003667 | >mml-miR-488 | MIMAT0006348 |
| >ath-miR395f | MIMAT0000943 | >xtr-miR-16b | MIMAT0003668 | >mml-miR-489 | MIMAT0006349 |
| >ath-miR396a | MIMAT0000944 | >xtr-miR-20a | MIMAT0003669 | >mml-miR-490-5p | MIMAT0006350 |
| >ath-miR396b | MIMAT0000945 | >xtr-miR-22 | MIMAT0003672 | >mml-miR-490-3p | MIMAT0006351 |
| >ath-miR397a | MIMAT0000946 | >xtr-miR-23b | MIMAT0003673 | >mml-miR-491-5p | MIMAT0006352 |
| >ath-miR397b | MIMAT0000947 | >xtr-miR-25 | MIMAT0003674 | >mml-miR-491-3p | MIMAT0006353 |
| >ath-miR398a | MIMAT0000948 | >xtr-miR-27c | MIMAT0003675 | >mml-miR-492 | MIMAT0006354 |
| >ath-miR398b | MIMAT0000949 | >xtr-miR-29c | MIMAT0003677 | >mml-miR-493 | MIMAT0006355 |
| >ath-miR398c | MIMAT0000950 | >xtr-miR-30e | MIMAT0003678 | >mml-miR-494 | MIMAT0006356 |
| >ath-miR399a | MIMAT0000951 | >xtr-miR-31 | MIMAT0003679 | >mml-miR-495 | MIMAT0006357 |
| >ath-miR399b | MIMAT0000952 | >xtr-miR-33a | MIMAT0003680 | >mml-miR-496 | MIMAT0006358 |
| >ath-miR399c | MIMAT0000953 | >xtr-miR-33b | MIMAT0003681 | >mml-miR-497 | MIMAT0006359 |
| >ath-miR399d | MIMAT0000954 | >xtr-miR-100 | MIMAT0003682 | >mml-miR-498 | MIMAT0006360 |
| >ath-miR399e | MIMAT0000955 | >xtr-miR-124 | MIMAT0003683 | >mml-miR-499-5p | MIMAT0006361 |
| >ath-miR399f | MIMAT0000956 | >xtr-miR-125a | MIMAT0003684 | >mml-miR-499-3p | MIMAT0006362 |
| >osa-miR393 | MIMAT0000957 | >xtr-miR-137 | MIMAT0003685 | >mml-miR-500 | MIMAT0006363 |
| >osa-miR394 | MIMAT0000958 | >xtr-miR-143 | MIMAT0003686 | >mml-miR-501 | MIMAT0006364 |
| >osa-miR395b | MIMAT0000959 | >xtr-miR-144 | MIMAT0003687 | >mml-miR-502-5p | MIMAT0006365 |
| >osa-miR395d | MIMAT0000960 | >xtr-miR-145 | MIMAT0003688 | >mml-miR-502-3p | MIMAT0006366 |
| >osa-miR395e | MIMAT0000961 | >xtr-miR-191 | MIMAT0003689 | >mml-miR-503 | MIMAT0006367 |
| >osa-miR395g | MIMAT0000962 | >xtr-miR-196a | MIMAT0003690 | >mml-miR-504 | MIMAT0006368 |
| >osa-miR395h | MIMAT0000963 | >xtr-miR-196b | MIMAT0003691 | >mml-miR-505 | MIMAT0006369 |
| >osa-miR395i | MIMAT0000964 | >xtr-miR-199b | MIMAT0003692 | >mml-miR-511 | MIMAT0006370 |
| >osa-miR395j | MIMAT0000965 | >xtr-miR-200a | MIMAT0003693 | >mml-miR-512-5p | MIMAT0006371 |
| >osa-miR395k | MIMAT0000966 | >xtr-miR-200b | MIMAT0003694 | >mml-miR-512-3p | MIMAT0006372 |
| >osa-miR395l | MIMAT0000967 | >xtr-miR-203 | MIMAT0003695 | >mml-miR-516a-5p | MIMAT0006373 |
| >osa-miR395s | MIMAT0000968 | >xtr-miR-204 | MIMAT0003696 | >mml-miR-516a-3p | MIMAT0006374 |
| >osa-miR395t | MIMAT0000969 | >xtr-miR-205a | MIMAT0003697 | >mml-miR-517a | MIMAT0006375 |
| >osa-miR395c | MIMAT0000972 | >xtr-miR-208 | MIMAT0003698 | >mml-miR-517b | MIMAT0006376 |
| >osa-miR395a | MIMAT0000973 | >xtr-miR-212 | MIMAT0003699 | >mml-miR-518a-5p | MIMAT0006377 |
| >osa-miR395f | MIMAT0000974 | >xtr-miR-221 | MIMAT0003700 | >mml-miR-518a-3p | MIMAT0006378 |
| >osa-miR395u | MIMAT0000975 | >xtr-miR-363-5p | MIMAT0003701 | >mml-miR-518b | MIMAT0006379 |
| >osa-miR396a | MIMAT0000977 | >xtr-miR-363-3p | MIMAT0003702 | >mml-miR-518c | MIMAT0006380 |
| >osa-miR396b | MIMAT0000978 | >xtr-miR-429 | MIMAT0003703 | >mml-miR-518d | MIMAT0006381 |
| >osa-miR396c | MIMAT0000979 | >xtr-miR-449 | MIMAT0003704 | >mml-miR-518e | MIMAT0006382 |
| >osa-miR397a | MIMAT0000980 | >xtr-miR-451 | MIMAT0003705 | >mml-miR-518f | MIMAT0006383 |
| >osa-miR397b | MIMAT0000981 | >xtr-miR-18b | MIMAT0003706 | >mml-miR-519a | MIMAT0006384 |
| >osa-miR398a | MIMAT0000982 | >xtr-miR-20b | MIMAT0003707 | >mml-miR-519b | MIMAT0006385 |
| >osa-miR398b | MIMAT0000983 | >xtr-miR-133a | MIMAT0003708 | >mml-miR-519c | MIMAT0006386 |
| >osa-miR399a | MIMAT0000984 | >xtr-miR-428 | MIMAT0003709 | >mml-miR-519d | MIMAT0006387 |
| >osa-miR399b | MIMAT0000985 | >mmu-miR-652 | MIMAT0003711 | >mml-miR-520a | MIMAT0006388 |
| >osa-miR399c | MIMAT0000986 | >tni-miR-10d | MIMAT0003772 | >mml-miR-520b | MIMAT0006389 |
| >osa-miR399d | MIMAT0000987 | >fru-miR-10d | MIMAT0003773 | >mml-miR-520c | MIMAT0006390 |
| >osa-miR399e | MIMAT0000988 | >bmo-let-7 | MIMAT0004190 | >mml-miR-520d-5p | MIMAT0006391 |
| >osa-miR399f | MIMAT0000989 | >bmo-miR-1 | MIMAT0004191 | >mml-miR-520d-3p | MIMAT0006392 |
| >osa-miR399g | MIMAT0000990 | >bmo-miR-7 | MIMAT0004192 | >mml-miR-520e | MIMAT0006393 |
| >osa-miR399h | MIMAT0000991 | >bmo-miR-8 | MIMAT0004193 | >mml-miR-520f | MIMAT0006394 |
| >osa-miR399i | MIMAT0000992 | >bmo-miR-9 | MIMAT0004194 | >mml-miR-520g | MIMAT0006395 |
| >osa-miR399j | MIMAT0000993 | >bmo-miR-10 | MIMAT0004195 | >mml-miR-520h | MIMAT0006396 |
| >osa-miR399k | MIMAT0000994 | >bmo-miR-14 | MIMAT0004196 | >mml-miR-521 | MIMAT0006397 |
| >ebv-miR-BHRF1-1 | MIMAT0000995 | >bmo-miR-34 | MIMAT0004197 | >mml-miR-522 | MIMAT0006398 |
| >ebv-miR-BHRF1-2 | MIMAT0000997 | >bmo-miR-124 | MIMAT0004198 | >mml-miR-523a | MIMAT0006399 |
| >ebv-miR-BHRF1-3 | MIMAT0000998 | >bmo-miR-263b | MIMAT0004199 | >mml-miR-523b | MIMAT0006400 |
| >ebv-miR-BART1-5p | MIMAT0000999 | >bmo-miR-263a | MIMAT0004200 | >mml-miR-523c | MIMAT0006401 |
| >ebv-miR-BART1-3p | MIMAT0003390 | >bmo-miR-275 | MIMAT0004201 | >mml-miR-525 | MIMAT0006402 |
| >ebv-miR-BART2-5p | MIMAT0001000 | >bmo-miR-276 | MIMAT0004202 | >mml-miR-532-5p | MIMAT0006403 |
| >ebv-miR-BART2-3p | MIMAT0004744 | >bmo-miR-277 | MIMAT0004203 | >mml-miR-532-3p | MIMAT0006404 |
| >ath-miR400 | MIMAT0001001 | >bmo-miR-279 | MIMAT0004204 | >mml-miR-539 | MIMAT0006405 |
| >ath-miR401 | MIMAT0001002 | >bmo-miR-282 | MIMAT0004205 | >mml-miR-542-5p | MIMAT0006406 |
| >ath-miR402 | MIMAT0001003 | >bmo-miR-283 | MIMAT0004206 | >mml-miR-542-3p | MIMAT0006407 |
| >ath-miR403 | MIMAT0001004 | >bmo-miR-305 | MIMAT0004207 | >mml-miR-544 | MIMAT0006408 |
| >ath-miR404 | MIMAT0001005 | >bmo-miR-307 | MIMAT0004208 | >mml-miR-545 | MIMAT0006409 |
| >ath-miR405a | MIMAT0001006 | >kshv-miR-K12-12 | MIMAT0003712 | >mml-miR-548a | MIMAT0006410 |
| >ath-miR405b | MIMAT0001007 | >ebv-miR-BART15 | MIMAT0003713 | >mml-miR-548b | MIMAT0006411 |
| >ath-miR405d | MIMAT0001008 | >ebv-miR-BART16 | MIMAT0003714 | >mml-miR-548c | MIMAT0006412 |
| >ath-miR406 | MIMAT0001009 | >ebv-miR-BART17-5p | MIMAT0003715 | >mml-miR-548d-5p | MIMAT0006413 |
| >ath-miR407 | MIMAT0001010 | >ebv-miR-BART17-3p | MIMAT0003716 | >mml-miR-548d-3p | MIMAT0006414 |
| >ath-miR408 | MIMAT0001011 | >ebv-miR-BART18-5p | MIMAT0003717 | >mml-miR-548e | MIMAT0006415 |
| >ath-miR156g | MIMAT0001012 | >ebv-miR-BART18-3p | MIMAT0004835 | >mml-miR-548f | MIMAT0006416 |
| >ath-miR156h | MIMAT0001013 | >ebv-miR-BART19-5p | MIMAT0004836 | >mml-miR-549 | MIMAT0006417 |
| >ath-miR158b | MIMAT0001014 | >ebv-miR-BART19-3p | MIMAT0003718 | >mml-miR-550 | MIMAT0006418 |
| >ath-miR159c | MIMAT0001015 | >ebv-miR-BART20-5p | MIMAT0003719 | >mml-miR-551a | MIMAT0006419 |
| >ath-miR319c | MIMAT0001016 | >ebv-miR-BART20-3p | MIMAT0003720 | >mml-miR-551b | MIMAT0006420 |
| >ath-miR164c | MIMAT0001017 | >gga-miR-21 | MIMAT0003774 | >mml-miR-552 | MIMAT0006421 |
| >ath-miR167c | MIMAT0001018 | >gga-miR-451 | MIMAT0003775 | >mml-miR-553 | MIMAT0006422 |
| >ath-miR172e | MIMAT0001019 | >gga-miR-144 | MIMAT0003776 | >mml-miR-554 | MIMAT0006423 |
| >osa-miR156k | MIMAT0001020 | >gga-miR-456 | MIMAT0003777 | >mml-miR-556-5p | MIMAT0006424 |
| >osa-miR156l | MIMAT0001021 | >gga-miR-460 | MIMAT0003778 | >mml-miR-556-3p | MIMAT0006425 |
| >osa-miR159a | MIMAT0001022 | >gga-miR-757 | MIMAT0003779 | >mml-miR-557 | MIMAT0006426 |
| >osa-miR159b | MIMAT0001023 | >mmu-miR-490 | MIMAT0003780 | >mml-miR-558 | MIMAT0006427 |
| >osa-miR159c | MIMAT0001024 | >mmu-miR-676 | MIMAT0003782 | >mml-miR-562 | MIMAT0006428 |
| >osa-miR159d | MIMAT0001025 | >mmu-miR-615-5p | MIMAT0004837 | >mml-miR-563 | MIMAT0006429 |
| >osa-miR159e | MIMAT0001026 | >mmu-miR-615-3p | MIMAT0003783 | >mml-miR-567 | MIMAT0006430 |
| >osa-miR159f | MIMAT0001027 | >bta-miR-106 | MIMAT0003784 | >mml-miR-568 | MIMAT0006431 |
| >osa-miR319a | MIMAT0001028 | >bta-miR-107 | MIMAT0003785 | >mml-miR-569 | MIMAT0006432 |
| >osa-miR319b | MIMAT0001029 | >bta-miR-10a | MIMAT0003786 | >mml-miR-570 | MIMAT0006433 |
| >osa-miR160e | MIMAT0001030 | >bta-miR-127 | MIMAT0003787 | >mml-miR-572 | MIMAT0006434 |
| >osa-miR160f | MIMAT0001031 | >bta-miR-139 | MIMAT0003788 | >mml-miR-573 | MIMAT0006435 |
| >osa-miR162b | MIMAT0001032 | >bta-miR-140 | MIMAT0003789 | >mml-miR-576-5p | MIMAT0006436 |
| >osa-miR164c | MIMAT0001033 | >bta-miR-142 | MIMAT0003790 | >mml-miR-576-3p | MIMAT0006437 |
| >osa-miR164d | MIMAT0001034 | >bta-miR-15b | MIMAT0003792 | >mml-miR-577 | MIMAT0006438 |
| >osa-miR164e | MIMAT0001035 | >bta-miR-181b | MIMAT0003793 | >mml-miR-578 | MIMAT0006439 |
| >osa-miR166j | MIMAT0001036 | >bta-miR-193a | MIMAT0003795 | >mml-miR-579 | MIMAT0006440 |
| >osa-miR166k | MIMAT0001037 | >bta-miR-20b | MIMAT0003796 | >mml-miR-580 | MIMAT0006441 |
| >osa-miR166l | MIMAT0001038 | >bta-miR-215 | MIMAT0003797 | >mml-miR-581 | MIMAT0006442 |
| >osa-miR167d | MIMAT0001039 | >bta-miR-218 | MIMAT0003798 | >mml-miR-582-5p | MIMAT0006443 |
| >osa-miR167e | MIMAT0001040 | >bta-miR-30e-5p | MIMAT0003799 | >mml-miR-582-3p | MIMAT0006444 |
| >osa-miR167f | MIMAT0001041 | >bta-miR-345 | MIMAT0003800 | >mml-miR-583 | MIMAT0006445 |
| >osa-miR167g | MIMAT0001042 | >bta-miR-369-5p | MIMAT0003801 | >mml-miR-584 | MIMAT0006446 |
| >osa-miR167h | MIMAT0001043 | >bta-miR-369-3p | MIMAT0003802 | >mml-miR-586 | MIMAT0006447 |
| >osa-miR167i | MIMAT0001044 | >bta-miR-380-5p | MIMAT0003803 | >mml-miR-587 | MIMAT0006448 |
| >osa-miR168a | MIMAT0001045 | >bta-miR-380-3p | MIMAT0003804 | >mml-miR-589 | MIMAT0006449 |
| >osa-miR168b | MIMAT0001046 | >bta-miR-487a | MIMAT0003805 | >mml-miR-590-5p | MIMAT0006450 |
| >osa-miR169b | MIMAT0001047 | >bta-miR-545 | MIMAT0003807 | >mml-miR-590-3p | MIMAT0006451 |
| >osa-miR169c | MIMAT0001048 | >bta-miR-92 | MIMAT0003808 | >mml-miR-592 | MIMAT0006452 |
| >osa-miR169d | MIMAT0001049 | >bta-miR-98 | MIMAT0003809 | >mml-miR-593 | MIMAT0006453 |
| >osa-miR169e | MIMAT0001050 | >bta-let-7d | MIMAT0003810 | >mml-miR-597 | MIMAT0006454 |
| >osa-miR169f | MIMAT0001051 | >bta-miR-124a | MIMAT0003811 | >mml-miR-598 | MIMAT0006455 |
| >osa-miR169g | MIMAT0001052 | >bta-miR-132 | MIMAT0003812 | >mml-miR-599 | MIMAT0006456 |
| >osa-miR169h | MIMAT0001053 | >bta-miR-138 | MIMAT0003813 | >mml-miR-600 | MIMAT0006457 |
| >osa-miR169i | MIMAT0001054 | >bta-miR-148b | MIMAT0003814 | >mml-miR-601 | MIMAT0006458 |
| >osa-miR169j | MIMAT0001055 | >bta-miR-17-5p | MIMAT0003815 | >mml-miR-604 | MIMAT0006459 |
| >osa-miR169k | MIMAT0001056 | >bta-miR-17-3p | MIMAT0003816 | >mml-miR-605 | MIMAT0006460 |
| >osa-miR169l | MIMAT0001057 | >bta-miR-181c | MIMAT0003817 | >mml-miR-607 | MIMAT0006461 |
| >osa-miR169m | MIMAT0001058 | >bta-miR-186 | MIMAT0003818 | >mml-miR-609 | MIMAT0006462 |
| >osa-miR169n | MIMAT0001059 | >bta-miR-191 | MIMAT0003819 | >mml-miR-611 | MIMAT0006463 |
| >osa-miR169o | MIMAT0001060 | >bta-miR-192 | MIMAT0003820 | >mml-miR-612 | MIMAT0006464 |
| >osa-miR169p | MIMAT0001061 | >bta-miR-199b | MIMAT0003821 | >mml-miR-615-5p | MIMAT0006465 |
| >osa-miR169q | MIMAT0001062 | >bta-miR-200a | MIMAT0003822 | >mml-miR-615-3p | MIMAT0006466 |
| >osa-miR171b | MIMAT0001063 | >bta-miR-200c | MIMAT0003823 | >mml-miR-616 | MIMAT0006467 |
| >osa-miR171c | MIMAT0001064 | >bta-miR-210 | MIMAT0003824 | >mml-miR-618 | MIMAT0006468 |
| >osa-miR171d | MIMAT0001065 | >bta-miR-214 | MIMAT0003825 | >mml-miR-619 | MIMAT0006469 |
| >osa-miR171e | MIMAT0001066 | >bta-miR-22-5p | MIMAT0003826 | >mml-miR-624 | MIMAT0006470 |
| >osa-miR171f | MIMAT0001067 | >bta-miR-23a | MIMAT0003827 | >mml-miR-625 | MIMAT0006471 |
| >osa-miR171g | MIMAT0001068 | >bta-miR-29b | MIMAT0003828 | >mml-miR-626 | MIMAT0006472 |
| >osa-miR172a | MIMAT0001069 | >bta-miR-29c | MIMAT0003829 | >mml-miR-627 | MIMAT0006473 |
| >osa-miR172b | MIMAT0001070 | >bta-miR-361 | MIMAT0003830 | >mml-miR-628-5p | MIMAT0006474 |
| >osa-miR172c | MIMAT0001071 | >bta-miR-423 | MIMAT0003831 | >mml-miR-628-3p | MIMAT0006475 |
| >osa-miR166g | MIMAT0001072 | >bta-miR-425-5p | MIMAT0003832 | >mml-miR-631 | MIMAT0006476 |
| >osa-miR166h | MIMAT0001073 | >bta-miR-425-3p | MIMAT0003833 | >mml-miR-632 | MIMAT0006477 |
| >osa-miR166i | MIMAT0001074 | >bta-miR-450 | MIMAT0003834 | >mml-miR-633 | MIMAT0006478 |
| >hsa-miR-384 | MIMAT0001075 | >bta-miR-455 | MIMAT0003835 | >mml-miR-636 | MIMAT0006479 |
| >mmu-miR-384-5p | MIMAT0004745 | >bta-miR-93 | MIMAT0003837 | >mml-miR-638 | MIMAT0006480 |
| >mmu-miR-384-3p | MIMAT0001076 | >bta-let-7g | MIMAT0003838 | >mml-miR-639 | MIMAT0006481 |
| >osa-miR171h | MIMAT0001077 | >bta-miR-10b | MIMAT0003839 | >mml-miR-640 | MIMAT0006482 |
| >osa-miR393b | MIMAT0001078 | >bta-miR-24-3p | MIMAT0003840 | >mml-miR-642 | MIMAT0006483 |
| >osa-miR408 | MIMAT0001079 | >bta-miR-30a-5p | MIMAT0003841 | >mml-miR-643 | MIMAT0006484 |
| >hsa-miR-196b | MIMAT0001080 | >bta-miR-200b | MIMAT0003842 | >mml-miR-644 | MIMAT0006485 |
| >mmu-miR-196b | MIMAT0001081 | >bta-miR-7 | MIMAT0003843 | >mml-miR-648 | MIMAT0006486 |
| >rno-miR-196b | MIMAT0001082 | >bta-let-7a | MIMAT0003844 | >mml-miR-649 | MIMAT0006487 |
| >osa-miR172d | MIMAT0001084 | >bta-miR-150 | MIMAT0003845 | >mml-miR-650a | MIMAT0006488 |
| >osa-miR171i | MIMAT0001085 | >bta-miR-342 | MIMAT0003846 | >mml-miR-650b | MIMAT0006489 |
| >osa-miR167j | MIMAT0001086 | >bta-miR-487b | MIMAT0003847 | >mml-miR-650c | MIMAT0006490 |
| >osa-miR166m | MIMAT0001087 | >bta-miR-532 | MIMAT0003848 | >mml-miR-650d | MIMAT0006491 |
| >osa-miR166n | MIMAT0001088 | >bta-miR-122 | MIMAT0003849 | >mml-miR-651 | MIMAT0006492 |
| >osa-miR164f | MIMAT0001089 | >bta-miR-30c | MIMAT0003850 | >mml-miR-652 | MIMAT0006493 |
| >mmu-miR-409-5p | MIMAT0004746 | >bta-let-7i | MIMAT0003851 | >mml-miR-653 | MIMAT0006494 |
| >mmu-miR-409-3p | MIMAT0001090 | >bta-miR-23b | MIMAT0003852 | >mml-miR-654-5p | MIMAT0006495 |
| >mmu-miR-410 | MIMAT0001091 | >bta-miR-25 | MIMAT0003853 | >mml-miR-654-3p | MIMAT0006496 |
| >mmu-miR-376b | MIMAT0001092 | >bta-miR-34c | MIMAT0003854 | >mml-miR-656 | MIMAT0006497 |
| >mmu-miR-411 | MIMAT0004747 | >bta-miR-363 | MIMAT0003855 | >mml-miR-657 | MIMAT0006498 |
| >mmu-miR-412 | MIMAT0001094 | >mtr-miR395c | MIMAT0003856 | >mml-miR-660 | MIMAT0006499 |
| >mmu-miR-370 | MIMAT0001095 | >mtr-miR395d | MIMAT0003857 | >mml-miR-661 | MIMAT0006500 |
| >gga-miR-29a | MIMAT0001096 | >mtr-miR395e | MIMAT0003858 | >mml-miR-662 | MIMAT0006501 |
| >gga-miR-29b | MIMAT0001097 | >mtr-miR395f | MIMAT0003859 | >mml-miR-663 | MIMAT0006502 |
| >gga-let-7i | MIMAT0001098 | >mtr-miR395g | MIMAT0003860 | >mml-miR-664 | MIMAT0006503 |
| >gga-miR-135a | MIMAT0001099 | >mtr-miR395h | MIMAT0003861 | >mml-miR-668 | MIMAT0006504 |
| >gga-miR-33 | MIMAT0001100 | >mtr-miR395i | MIMAT0003862 | >mml-miR-671-5p | MIMAT0006505 |
| >gga-let-7a | MIMAT0001101 | >mtr-miR395j | MIMAT0003863 | >mml-miR-671-3p | MIMAT0006506 |
| >gga-let-7b | MIMAT0001102 | >mtr-miR395k | MIMAT0003864 | >mml-miR-675 | MIMAT0006507 |
| >gga-miR-99a | MIMAT0001103 | >mtr-miR395l | MIMAT0003865 | >mml-miR-758 | MIMAT0006508 |
| >gga-let-7c | MIMAT0001104 | >mtr-miR395m | MIMAT0003866 | >mml-miR-765 | MIMAT0006509 |
| >gga-miR-125b | MIMAT0001105 | >mtr-miR395n | MIMAT0003867 | >mml-miR-767-5p | MIMAT0006510 |
| >gga-miR-155 | MIMAT0001106 | >mtr-miR395o | MIMAT0003868 | >mml-miR-767-3p | MIMAT0006511 |
| >gga-miR-222 | MIMAT0001107 | >mtr-miR395p | MIMAT0003869 | >mml-miR-770-5p | MIMAT0006514 |
| >gga-miR-221 | MIMAT0001108 | >osa-miR395m | MIMAT0003870 | >mml-miR-802 | MIMAT0006515 |
| >gga-miR-92 | MIMAT0001109 | >osa-miR395n | MIMAT0003871 | >mml-miR-874 | MIMAT0006516 |
| >gga-miR-19b | MIMAT0001110 | >osa-miR395o | MIMAT0003872 | >mml-miR-875-5p | MIMAT0006517 |
| >gga-miR-20a | MIMAT0001111 | >osa-miR395p | MIMAT0003873 | >mml-miR-875-3p | MIMAT0006518 |
| >gga-miR-19a | MIMAT0001112 | >osa-miR395q | MIMAT0003874 | >mml-miR-876-5p | MIMAT0006519 |
| >gga-miR-18a | MIMAT0001113 | >osa-miR395v | MIMAT0003876 | >mml-miR-876-3p | MIMAT0006520 |
| >gga-miR-17-5p | MIMAT0001114 | >osa-miR395w | MIMAT0003877 | >mml-miR-877 | MIMAT0006521 |
| >gga-miR-17-3p | MIMAT0001115 | >osa-miR395r | MIMAT0003878 | >mml-miR-885-5p | MIMAT0006522 |
| >gga-miR-16 | MIMAT0001116 | >mdv1-miR-M1 | MIMAT0003920 | >mml-miR-885-3p | MIMAT0006523 |
| >gga-miR-15a | MIMAT0001117 | >mdv1-miR-M2 | MIMAT0003921 | >mml-miR-886-5p | MIMAT0006524 |
| >gga-miR-26a | MIMAT0001118 | >mdv1-miR-M3 | MIMAT0003923 | >mml-miR-886-3p | MIMAT0006525 |
| >gga-miR-153 | MIMAT0001119 | >mdv1-miR-M4 | MIMAT0003924 | >mml-miR-887 | MIMAT0006526 |
| >gga-miR-148a | MIMAT0001120 | >mdv1-miR-M5 | MIMAT0003926 | >mml-miR-888 | MIMAT0006527 |
| >gga-miR-196 | MIMAT0001121 | >mdv1-miR-M6-5p | MIMAT0005803 | >mml-miR-889 | MIMAT0006528 |
| >gga-miR-138 | MIMAT0001122 | >mdv1-miR-M6-3p | MIMAT0003927 | >mml-miR-890 | MIMAT0006529 |
| >gga-miR-128 | MIMAT0001123 | >mdv1-miR-M7 | MIMAT0005804 | >mml-miR-891 | MIMAT0006530 |
| >gga-miR-187 | MIMAT0001124 | >mdv1-miR-M8-5p | MIMAT0005805 | >mml-miR-892 | MIMAT0006531 |
| >gga-miR-32 | MIMAT0001125 | >mdv1-miR-M8-3p | MIMAT0003929 | >mml-miR-920 | MIMAT0006532 |
| >gga-miR-133a | MIMAT0001126 | >ath-miR771 | MIMAT0003930 | >mml-miR-922 | MIMAT0006533 |
| >gga-miR-1a | MIMAT0001127 | >ath-miR472 | MIMAT0003931 | >mml-miR-924 | MIMAT0006534 |
| >gga-miR-124a | MIMAT0001128 | >ath-miR773 | MIMAT0003932 | >mml-miR-933 | MIMAT0006535 |
| >gga-miR-30d | MIMAT0001129 | >ath-miR774 | MIMAT0003933 | >mml-miR-934 | MIMAT0006536 |
| >gga-miR-30b | MIMAT0001130 | >ath-miR775 | MIMAT0003934 | >mml-miR-936 | MIMAT0006537 |
| >gga-miR-216 | MIMAT0001131 | >ath-miR776 | MIMAT0003935 | >mml-miR-937 | MIMAT0006538 |
| >gga-miR-217 | MIMAT0001132 | >ath-miR777 | MIMAT0003936 | >mml-miR-938 | MIMAT0006539 |
| >gga-miR-194 | MIMAT0001133 | >ath-miR778 | MIMAT0003937 | >mml-miR-939 | MIMAT0006540 |
| >gga-miR-215 | MIMAT0001134 | >ath-miR779.2 | MIMAT0004329 | >mml-miR-940 | MIMAT0006541 |
| >gga-miR-30a-5p | MIMAT0001135 | >ath-miR779.1 | MIMAT0003938 | >mml-miR-942 | MIMAT0006542 |
| >gga-miR-30a-3p | MIMAT0001136 | >ath-miR780.1 | MIMAT0004218 | >mml-miR-944 | MIMAT0006543 |
| >gga-miR-30c | MIMAT0001137 | >ath-miR780.2 | MIMAT0003939 | >vvi-miR156h | MIMAT0006544 |
| >gga-miR-133b | MIMAT0001138 | >ath-miR781 | MIMAT0003940 | >vvi-miR169b | MIMAT0006545 |
| >gga-miR-206 | MIMAT0001139 | >ath-miR782 | MIMAT0003941 | >vvi-miR169h | MIMAT0006546 |
| >gga-miR-223 | MIMAT0001140 | >ath-miR783 | MIMAT0003942 | >vvi-miR169i | MIMAT0006547 |
| >gga-miR-18b | MIMAT0001141 | >ptc-miR171k | MIMAT0003943 | >vvi-miR169l | MIMAT0006548 |
| >gga-miR-106 | MIMAT0001142 | >ptc-miR403c | MIMAT0003944 | >vvi-miR169n | MIMAT0006549 |
| >gga-miR-302a | MIMAT0001143 | >hsa-miR-765 | MIMAT0003945 | >vvi-miR169o | MIMAT0006550 |
| >gga-miR-218 | MIMAT0001144 | >hsa-miR-770-5p | MIMAT0003948 | >vvi-miR169q | MIMAT0006551 |
| >gga-miR-103 | MIMAT0001145 | >sme-bantam-a | MIMAT0003949 | >vvi-miR169v | MIMAT0006552 |
| >gga-miR-203 | MIMAT0001146 | >sme-bantam-b | MIMAT0003950 | >vvi-miR169w | MIMAT0006553 |
| >gga-miR-107 | MIMAT0001147 | >sme-bantam-c | MIMAT0003951 | >vvi-miR169x | MIMAT0006554 |
| >gga-miR-10b | MIMAT0001148 | >sme-let-7a | MIMAT0003952 | >vvi-miR171g | MIMAT0006555 |
| >gga-miR-181a | MIMAT0001168 | >sme-let-7b | MIMAT0003953 | >vvi-miR319e | MIMAT0006556 |
| >gga-miR-181b | MIMAT0001151 | >sme-let-7c | MIMAT0003955 | >vvi-miR393a | MIMAT0006557 |
| >gga-miR-199 | MIMAT0001152 | >sme-lin-4 | MIMAT0003956 | >vvi-miR394c | MIMAT0006558 |
| >gga-miR-137 | MIMAT0001153 | >sme-miR-1a | MIMAT0003960 | >vvi-miR395n | MIMAT0006559 |
| >gga-miR-15b | MIMAT0001154 | >sme-miR-1b | MIMAT0003961 | >vvi-miR396c | MIMAT0006560 |
| >gga-miR-190 | MIMAT0001155 | >sme-miR-1c | MIMAT0003962 | >vvi-miR397a | MIMAT0006561 |
| >gga-miR-204 | MIMAT0001156 | >sme-miR-2a | MIMAT0003963 | >vvi-miR397b | MIMAT0006562 |
| >gga-miR-7 | MIMAT0001157 | >sme-miR-2b | MIMAT0003966 | >vvi-miR398b | MIMAT0006563 |
| >gga-miR-184 | MIMAT0001158 | >sme-miR-2c | MIMAT0003967 | >vvi-miR398c | MIMAT0006564 |
| >gga-miR-140 | MIMAT0001159 | >sme-miR-2d | MIMAT0003968 | >vvi-miR399c | MIMAT0006565 |
| >gga-let-7g | MIMAT0001160 | >sme-miR-7a | MIMAT0003969 | >vvi-miR399d | MIMAT0006566 |
| >gga-let-7d | MIMAT0001161 | >sme-miR-7b | MIMAT0003970 | >vvi-miR399f | MIMAT0006567 |
| >gga-let-7f | MIMAT0001162 | >sme-miR-7c | MIMAT0003971 | >vvi-miR399i | MIMAT0006568 |
| >gga-miR-146a | MIMAT0001163 | >sme-miR-8 | MIMAT0003972 | >vvi-miR403a | MIMAT0006569 |
| >gga-miR-205b | MIMAT0001164 | >sme-miR-10 | MIMAT0003973 | >vvi-miR403b | MIMAT0006570 |
| >gga-miR-130b | MIMAT0001165 | >sme-miR-12 | MIMAT0003975 | >vvi-miR403c | MIMAT0006571 |
| >gga-miR-301 | MIMAT0001166 | >sme-miR-13 | MIMAT0003976 | >vvi-miR403d | MIMAT0006572 |
| >gga-miR-130a | MIMAT0001167 | >sme-miR-745 | MIMAT0003977 | >vvi-miR403e | MIMAT0006573 |
| >gga-miR-126 | MIMAT0001169 | >sme-miR-746 | MIMAT0003978 | >vvi-miR403f | MIMAT0006574 |
| >gga-miR-219 | MIMAT0001170 | >sme-miR-31a | MIMAT0003979 | >vvi-miR477 | MIMAT0006575 |
| >gga-miR-200a | MIMAT0001171 | >sme-miR-31b | MIMAT0003980 | >vvi-miR482 | MIMAT0006576 |
| >gga-miR-200b | MIMAT0001172 | >sme-miR-36 | MIMAT0003982 | >vvi-miR828a | MIMAT0006577 |
| >gga-miR-34a | MIMAT0001173 | >sme-miR-61 | MIMAT0003983 | >vvi-miR828b | MIMAT0006578 |
| >gga-miR-124b | MIMAT0001174 | >sme-miR-71a | MIMAT0003984 | >vvi-miR845a | MIMAT0006579 |
| >gga-miR-1b | MIMAT0001175 | >sme-miR-71b | MIMAT0003985 | >vvi-miR845b | MIMAT0006580 |
| >gga-miR-133c | MIMAT0001176 | >sme-miR-71c | MIMAT0003987 | >vvi-miR845c | MIMAT0006581 |
| >gga-miR-30e | MIMAT0001177 | >sme-miR-79 | MIMAT0003988 | >vvi-miR845d | MIMAT0006582 |
| >gga-miR-100 | MIMAT0001178 | >sme-miR-87a | MIMAT0003989 | >vvi-miR845e | MIMAT0006583 |
| >gga-miR-34b | MIMAT0001179 | >sme-miR-87b | MIMAT0003990 | >cel-miR-1817 | MIMAT0006584 |
| >gga-miR-34c | MIMAT0001180 | >sme-miR-92 | MIMAT0003991 | >cel-miR-1818 | MIMAT0006585 |
| >gga-let-7j | MIMAT0001181 | >sme-miR-124a | MIMAT0003992 | >cel-miR-1819 | MIMAT0006586 |
| >gga-let-7k | MIMAT0001182 | >sme-miR-124b | MIMAT0003993 | >cel-miR-1820 | MIMAT0006587 |
| >gga-miR-29c | MIMAT0001183 | >sme-miR-124c | MIMAT0003995 | >cel-miR-1821 | MIMAT0006588 |
| >gga-miR-205a | MIMAT0001184 | >sme-miR-125a | MIMAT0003996 | >cel-miR-1822 | MIMAT0006589 |
| >gga-miR-101 | MIMAT0001185 | >sme-miR-125b | MIMAT0003998 | >cel-miR-1823 | MIMAT0006590 |
| >gga-miR-23b | MIMAT0001186 | >sme-miR-133 | MIMAT0003999 | >cel-miR-1824 | MIMAT0006591 |
| >gga-miR-27b | MIMAT0001187 | >sme-miR-184 | MIMAT0004000 | >cfa-miR-216 | MIMAT0006592 |
| >gga-miR-24 | MIMAT0001188 | >sme-miR-190a | MIMAT0004001 | >cfa-miR-33 | MIMAT0006593 |
| >gga-miR-31 | MIMAT0001189 | >sme-miR-190b | MIMAT0004003 | >cfa-let-7a | MIMAT0006594 |
| >gga-miR-122 | MIMAT0001190 | >sme-miR-747 | MIMAT0004005 | >cfa-miR-26a | MIMAT0006595 |
| >gga-miR-183 | MIMAT0001191 | >sme-miR-219 | MIMAT0004006 | >cfa-miR-1835 | MIMAT0006596 |
| >gga-miR-7b | MIMAT0001192 | >sme-miR-277a | MIMAT0004007 | >cfa-miR-32 | MIMAT0006597 |
| >gga-miR-142-5p | MIMAT0001193 | >sme-miR-277b | MIMAT0004009 | >cfa-miR-204 | MIMAT0006598 |
| >gga-miR-142-3p | MIMAT0001194 | >sme-miR-277c | MIMAT0004010 | >cfa-miR-31 | MIMAT0006599 |
| >gga-miR-9 | MIMAT0001195 | >sme-miR-278 | MIMAT0004011 | >cfa-miR-101 | MIMAT0006600 |
| >dps-bantam | MIMAT0001197 | >sme-miR-281 | MIMAT0004013 | >cfa-miR-371 | MIMAT0007747 |
| >dps-let-7 | MIMAT0001198 | >sme-miR-67 | MIMAT0004014 | >cfa-miR-491 | MIMAT0006601 |
| >dps-miR-1 | MIMAT0001199 | >sme-miR-748 | MIMAT0004015 | >cfa-miR-150 | MIMAT0006602 |
| >dps-miR-2a | MIMAT0001200 | >sme-miR-749 | MIMAT0004016 | >cfa-miR-455 | MIMAT0006603 |
| >dps-miR-2b | MIMAT0001201 | >sme-miR-750 | MIMAT0004017 | >cfa-miR-30a | MIMAT0006604 |
| >dps-miR-2c | MIMAT0001202 | >sme-miR-751 | MIMAT0004018 | >cfa-miR-30c | MIMAT0006605 |
| >dps-miR-3 | MIMAT0001203 | >sme-miR-752 | MIMAT0004019 | >cfa-miR-206 | MIMAT0006606 |
| >dps-miR-4 | MIMAT0001204 | >sme-miR-753 | MIMAT0004020 | >cfa-miR-99b | MIMAT0006607 |
| >dps-miR-iab-4-5p | MIMAT0001205 | >sme-miR-754 | MIMAT0004021 | >cfa-let-7e | MIMAT0006608 |
| >dps-miR-iab-4-3p | MIMAT0001206 | >sme-miR-755 | MIMAT0004022 | >cfa-miR-125a | MIMAT0006609 |
| >dps-miR-5 | MIMAT0001207 | >sme-miR-756 | MIMAT0004023 | >cfa-let-7f | MIMAT0006610 |
| >dps-miR-6 | MIMAT0001208 | >sme-miR-277d | MIMAT0004024 | >cfa-miR-219 | MIMAT0006611 |
| >dps-miR-7 | MIMAT0001209 | >cel-miR-784 | MIMAT0004219 | >cfa-miR-23b | MIMAT0006612 |
| >dps-miR-8 | MIMAT0001210 | >cel-miR-785 | MIMAT0004220 | >cfa-miR-27b | MIMAT0006613 |
| >dps-miR-9a | MIMAT0001211 | >cel-miR-786 | MIMAT0004221 | >cfa-miR-24 | MIMAT0006614 |
| >dps-miR-9b | MIMAT0001212 | >cel-miR-787 | MIMAT0004222 | >cfa-miR-151 | MIMAT0006615 |
| >dps-miR-9c | MIMAT0001213 | >cel-miR-788 | MIMAT0004223 | >cfa-miR-30d | MIMAT0006616 |
| >dps-miR-10 | MIMAT0001214 | >cel-miR-789 | MIMAT0004224 | >cfa-miR-30b | MIMAT0006617 |
| >dps-miR-11 | MIMAT0001215 | >cel-miR-790 | MIMAT0004225 | >cfa-miR-1836 | MIMAT0006618 |
| >dps-miR-12 | MIMAT0001216 | >cel-miR-791 | MIMAT0004226 | >cfa-miR-122 | MIMAT0006619 |
| >dps-miR-13a | MIMAT0001217 | >cel-miR-792 | MIMAT0004227 | >cfa-miR-196b | MIMAT0006620 |
| >dps-miR-13b | MIMAT0001218 | >cel-miR-793 | MIMAT0004228 | >cfa-miR-183 | MIMAT0006621 |
| >dps-miR-14 | MIMAT0001219 | >cel-miR-794 | MIMAT0004229 | >cfa-miR-148a | MIMAT0006622 |
| >dps-miR-31a | MIMAT0001220 | >cel-miR-795 | MIMAT0004230 | >cfa-miR-129 | MIMAT0006623 |
| >dps-miR-31b | MIMAT0001221 | >cel-miR-796 | MIMAT0004231 | >cfa-miR-335 | MIMAT0006624 |
| >dps-miR-33 | MIMAT0001222 | >cel-miR-797 | MIMAT0004232 | >cfa-miR-29b | MIMAT0006625 |
| >dps-miR-34 | MIMAT0001223 | >cel-miR-798 | MIMAT0004233 | >cfa-miR-29a | MIMAT0006626 |
| >dps-miR-79 | MIMAT0001224 | >cel-miR-799 | MIMAT0004234 | >cfa-miR-30e | MIMAT0006627 |
| >dps-miR-87 | MIMAT0001225 | >cel-miR-800 | MIMAT0004235 | >cfa-miR-135 | MIMAT0006628 |
| >dps-miR-92a | MIMAT0001226 | >mmu-miR-804 | MIMAT0004210 | >cfa-miR-383 | MIMAT0006629 |
| >dps-miR-92b | MIMAT0001227 | >mmu-miR-805 | MIMAT0004211 | >cfa-miR-1837 | MIMAT0006630 |
| >dps-miR-100 | MIMAT0001228 | >mmu-miR-741 | MIMAT0004236 | >cfa-miR-130a | MIMAT0006631 |
| >dps-miR-124 | MIMAT0001229 | >mmu-miR-742 | MIMAT0004237 | >cfa-miR-192 | MIMAT0006632 |
| >dps-miR-125 | MIMAT0001230 | >mmu-miR-743a | MIMAT0004238 | >cfa-miR-128 | MIMAT0006633 |
| >dps-miR-133 | MIMAT0001231 | >osa-miR807a | MIMAT0004025 | >cfa-miR-7 | MIMAT0006634 |
| >dps-miR-184 | MIMAT0001232 | >osa-miR806a | MIMAT0004026 | >cfa-miR-181c | MIMAT0006635 |
| >dps-miR-210 | MIMAT0001233 | >osa-miR806b | MIMAT0004027 | >cfa-miR-181d | MIMAT0006636 |
| >dps-miR-219 | MIMAT0001234 | >osa-miR806c | MIMAT0004028 | >cfa-let-7g | MIMAT0006637 |
| >dps-miR-263a | MIMAT0001235 | >osa-miR806d | MIMAT0004029 | >cfa-miR-191 | MIMAT0006638 |
| >dps-miR-263b | MIMAT0001236 | >osa-miR806e | MIMAT0004030 | >cfa-miR-425 | MIMAT0006639 |
| >dps-miR-274 | MIMAT0001237 | >osa-miR806f | MIMAT0004031 | >cfa-miR-23a | MIMAT0006640 |
| >dps-miR-275 | MIMAT0001238 | >osa-miR806g | MIMAT0004032 | >cfa-miR-27a | MIMAT0006641 |
| >dps-miR-276a | MIMAT0001239 | >osa-miR806h | MIMAT0004033 | >cfa-miR-199 | MIMAT0006642 |
| >dps-miR-276b | MIMAT0001240 | >osa-miR807b | MIMAT0004034 | >cfa-miR-708 | MIMAT0006643 |
| >dps-miR-277 | MIMAT0001241 | >osa-miR807c | MIMAT0004035 | >cfa-miR-1838 | MIMAT0006644 |
| >dps-miR-278 | MIMAT0001242 | >osa-miR808 | MIMAT0004036 | >cfa-miR-139 | MIMAT0006645 |
| >dps-miR-279 | MIMAT0001243 | >osa-miR809a | MIMAT0004037 | >cfa-miR-138b | MIMAT0006646 |
| >dps-miR-280 | MIMAT0001244 | >osa-miR809b | MIMAT0004038 | >cfa-miR-15a | MIMAT0006647 |
| >dps-miR-281 | MIMAT0001245 | >osa-miR809c | MIMAT0004039 | >cfa-miR-16 | MIMAT0006648 |
| >dps-miR-282 | MIMAT0001246 | >osa-miR809d | MIMAT0004040 | >cfa-miR-17 | MIMAT0006649 |
| >dps-miR-283 | MIMAT0001247 | >osa-miR809e | MIMAT0004041 | >cfa-miR-19a | MIMAT0006650 |
| >dps-miR-284 | MIMAT0001248 | >osa-miR809f | MIMAT0004042 | >cfa-miR-20 | MIMAT0006651 |
| >dps-miR-285 | MIMAT0001249 | >osa-miR809g | MIMAT0004043 | >cfa-miR-19b | MIMAT0006652 |
| >dps-miR-286 | MIMAT0001250 | >osa-miR809h | MIMAT0004044 | >cfa-miR-92a | MIMAT0006653 |
| >dps-miR-287 | MIMAT0001251 | >osa-miR810a | MIMAT0004045 | >cfa-miR-138a | MIMAT0006654 |
| >dps-miR-288 | MIMAT0001252 | >osa-miR811a | MIMAT0004046 | >cfa-miR-499 | MIMAT0006655 |
| >dps-miR-289 | MIMAT0001253 | >osa-miR811b | MIMAT0004047 | >cfa-miR-1 | MIMAT0006656 |
| >dps-miR-304 | MIMAT0001254 | >osa-miR811c | MIMAT0004048 | >cfa-miR-124 | MIMAT0006657 |
| >dps-miR-305 | MIMAT0001255 | >osa-miR812a | MIMAT0004049 | >cfa-miR-320 | MIMAT0006658 |
| >dps-miR-306 | MIMAT0001256 | >osa-miR812b | MIMAT0004050 | >cfa-miR-130b | MIMAT0006659 |
| >dps-miR-307 | MIMAT0001257 | >osa-miR812c | MIMAT0004051 | >cfa-miR-185 | MIMAT0006660 |
| >dps-miR-308 | MIMAT0001258 | >osa-miR812d | MIMAT0004052 | >cfa-miR-1306 | MIMAT0006661 |
| >dps-miR-309 | MIMAT0001259 | >osa-miR812e | MIMAT0004053 | >cfa-miR-196a | MIMAT0006662 |
| >dps-miR-314 | MIMAT0001260 | >osa-miR813 | MIMAT0004054 | >cfa-miR-148b | MIMAT0006663 |
| >dps-miR-315 | MIMAT0001261 | >osa-miR814a | MIMAT0004055 | >cfa-miR-200c | MIMAT0006664 |
| >dps-miR-316 | MIMAT0001262 | >osa-miR814b | MIMAT0004056 | >cfa-miR-1307 | MIMAT0006665 |
| >dps-miR-317 | MIMAT0001263 | >osa-miR814c | MIMAT0004057 | >cfa-miR-107 | MIMAT0006666 |
| >dps-miR-318 | MIMAT0001264 | >osa-miR815a | MIMAT0004058 | >cfa-miR-146b | MIMAT0006667 |
| >dre-miR-7b | MIMAT0001265 | >osa-miR815b | MIMAT0004059 | >cfa-miR-99a | MIMAT0006668 |
| >dre-miR-7a | MIMAT0001266 | >osa-miR815c | MIMAT0004060 | >cfa-let-7c | MIMAT0006669 |
| >dre-miR-10a | MIMAT0001267 | >osa-miR816 | MIMAT0004061 | >cfa-miR-125b | MIMAT0006670 |
| >dre-miR-10b | MIMAT0001268 | >osa-miR817 | MIMAT0004062 | >cfa-miR-155 | MIMAT0006671 |
| >dre-miR-34 | MIMAT0001269 | >osa-miR818a | MIMAT0004063 | >cfa-miR-218 | MIMAT0006672 |
| >dre-miR-181b | MIMAT0001270 | >osa-miR818b | MIMAT0004064 | >cfa-miR-574 | MIMAT0006673 |
| >dre-miR-182 | MIMAT0001271 | >osa-miR818c | MIMAT0004065 | >cfa-miR-9 | MIMAT0006674 |
| >dre-miR-183 | MIMAT0001273 | >osa-miR818d | MIMAT0004066 | >cfa-miR-28 | MIMAT0006675 |
| >dre-miR-187 | MIMAT0001274 | >osa-miR818e | MIMAT0004067 | >cfa-miR-15b | MIMAT0006676 |
| >dre-miR-192 | MIMAT0001275 | >osa-miR819a | MIMAT0004068 | >cfa-miR-1839 | MIMAT0006677 |
| >dre-miR-196a | MIMAT0001276 | >osa-miR819b | MIMAT0004069 | >cfa-miR-26b | MIMAT0006678 |
| >dre-miR-199 | MIMAT0001277 | >osa-miR819c | MIMAT0004070 | >cfa-miR-1840 | MIMAT0006679 |
| >dre-miR-203a | MIMAT0001278 | >osa-miR819d | MIMAT0004071 | >cfa-miR-664 | MIMAT0006680 |
| >dre-miR-204 | MIMAT0001279 | >osa-miR819e | MIMAT0004072 | >cfa-miR-194 | MIMAT0006681 |
| >dre-miR-205 | MIMAT0001280 | >osa-miR819f | MIMAT0004073 | >cfa-miR-143 | MIMAT0006682 |
| >dre-miR-210 | MIMAT0001281 | >osa-miR819g | MIMAT0004074 | >cfa-miR-378 | MIMAT0006683 |
| >dre-miR-181a | MIMAT0001623 | >osa-miR819h | MIMAT0004075 | >cfa-miR-146a | MIMAT0006684 |
| >dre-miR-214 | MIMAT0001283 | >osa-miR819i | MIMAT0004076 | >cfa-miR-1271 | MIMAT0006685 |
| >dre-miR-216a | MIMAT0001284 | >osa-miR819j | MIMAT0004077 | >cfa-miR-1841 | MIMAT0006686 |
| >dre-miR-217 | MIMAT0001285 | >osa-miR819k | MIMAT0004078 | >cfa-miR-103 | MIMAT0006687 |
| >dre-miR-219 | MIMAT0001286 | >osa-miR820a | MIMAT0004079 | >cfa-miR-328 | MIMAT0006688 |
| >dre-miR-220 | MIMAT0001287 | >osa-miR820b | MIMAT0004080 | >cfa-miR-140 | MIMAT0006689 |
| >dre-miR-221 | MIMAT0001288 | >osa-miR820c | MIMAT0004081 | >cfa-miR-34a | MIMAT0006690 |
| >dre-miR-222 | MIMAT0001289 | >osa-miR821a | MIMAT0004082 | >cfa-miR-497 | MIMAT0006691 |
| >dre-miR-223 | MIMAT0001290 | >osa-miR821b | MIMAT0004083 | >cfa-miR-195 | MIMAT0006692 |
| >cbr-miR-353 | MIMAT0001291 | >osa-miR821c | MIMAT0004084 | >cfa-miR-34c | MIMAT0006693 |
| >cbr-miR-64 | MIMAT0001292 | >mdo-miR-1 | MIMAT0004085 | >cfa-miR-186 | MIMAT0006694 |
| >cbr-miR-231 | MIMAT0001293 | >mdo-miR-7 | MIMAT0004086 | >cfa-miR-106b | MIMAT0006695 |
| >cbr-miR-356 | MIMAT0001294 | >mdo-miR-9 | MIMAT0004087 | >cfa-miR-93 | MIMAT0006696 |
| >cbr-miR-83 | MIMAT0001295 | >mdo-miR-10a | MIMAT0004089 | >cfa-miR-25 | MIMAT0006697 |
| >cbr-miR-246 | MIMAT0001296 | >mdo-miR-10b | MIMAT0004090 | >cfa-miR-197 | MIMAT0006698 |
| >cbr-miR-51 | MIMAT0001297 | >mdo-miR-21 | MIMAT0004091 | >cfa-miR-193b | MIMAT0006699 |
| >cbr-miR-357 | MIMAT0001298 | >mdo-miR-22 | MIMAT0004092 | >cfa-miR-590 | MIMAT0006700 |
| >cbr-miR-253 | MIMAT0001299 | >mdo-miR-30a | MIMAT0004093 | >cfa-miR-1842 | MIMAT0006701 |
| >cbr-miR-70 | MIMAT0001300 | >mdo-miR-31 | MIMAT0004094 | >cfa-miR-137 | MIMAT0006702 |
| >cbr-miR-358 | MIMAT0001301 | >mdo-miR-32 | MIMAT0004095 | >cfa-miR-92b | MIMAT0006703 |
| >cbr-miR-61 | MIMAT0001302 | >mdo-miR-34a | MIMAT0004096 | >cfa-miR-350 | MIMAT0006704 |
| >cbr-miR-360 | MIMAT0001303 | >mdo-miR-100 | MIMAT0004097 | >cfa-miR-29c | MIMAT0006705 |
| >cbr-miR-239a | MIMAT0001304 | >mdo-miR-101 | MIMAT0004098 | >cfa-miR-1843 | MIMAT0006706 |
| >cbr-miR-249 | MIMAT0001305 | >mdo-miR-103 | MIMAT0004099 | >cfa-miR-181a | MIMAT0006707 |
| >cbr-miR-240 | MIMAT0001306 | >mdo-miR-107 | MIMAT0004100 | >cfa-miR-181b | MIMAT0006708 |
| >cbr-miR-254 | MIMAT0001307 | >mdo-miR-122 | MIMAT0004101 | >cfa-miR-342 | MIMAT0006709 |
| >cbr-miR-239b | MIMAT0001308 | >mdo-miR-124a | MIMAT0004102 | >cfa-miR-345 | MIMAT0006710 |
| >cbr-miR-62 | MIMAT0001309 | >mdo-miR-125b | MIMAT0004103 | >cfa-miR-493 | MIMAT0006711 |
| >cbr-miR-55 | MIMAT0001310 | >mdo-miR-128 | MIMAT0004104 | >cfa-miR-433 | MIMAT0006712 |
| >cbr-miR-84 | MIMAT0001311 | >mdo-miR-129 | MIMAT0004105 | >cfa-miR-127 | MIMAT0006713 |
| >cbr-miR-354 | MIMAT0001312 | >mdo-miR-130a | MIMAT0004106 | >cfa-miR-136 | MIMAT0006714 |
| >cbr-miR-35 | MIMAT0001313 | >mdo-miR-133a | MIMAT0004107 | >cfa-miR-379 | MIMAT0006715 |
| >cbr-miR-36 | MIMAT0001314 | >mdo-miR-135a | MIMAT0004108 | >cfa-miR-411 | MIMAT0006716 |
| >cbr-miR-38 | MIMAT0001315 | >mdo-miR-135b | MIMAT0004109 | >cfa-miR-380 | MIMAT0006717 |
| >cbr-miR-39 | MIMAT0001316 | >mdo-miR-137 | MIMAT0004110 | >cfa-miR-323 | MIMAT0006718 |
| >cbr-miR-40 | MIMAT0001317 | >mdo-miR-138 | MIMAT0004111 | >cfa-miR-329 | MIMAT0006719 |
| >cbr-miR-41 | MIMAT0001318 | >mdo-miR-142 | MIMAT0004112 | >cfa-miR-543 | MIMAT0006720 |
| >cbr-miR-355 | MIMAT0001319 | >mdo-miR-143 | MIMAT0004113 | >cfa-miR-495 | MIMAT0006721 |
| >rno-miR-421 | MIMAT0001320 | >mdo-miR-144 | MIMAT0004114 | >cfa-miR-376 | MIMAT0006722 |
| >ath-miR413 | MIMAT0001321 | >mdo-miR-451 | MIMAT0004115 | >cfa-miR-487 | MIMAT0006723 |
| >ath-miR414 | MIMAT0001322 | >mdo-miR-145 | MIMAT0004116 | >cfa-miR-382 | MIMAT0006724 |
| >ath-miR415 | MIMAT0001323 | >mdo-miR-152 | MIMAT0004117 | >cfa-miR-485 | MIMAT0006725 |
| >ath-miR416 | MIMAT0001324 | >mdo-miR-182 | MIMAT0004118 | >cfa-miR-409 | MIMAT0006726 |
| >ath-miR417 | MIMAT0001325 | >mdo-miR-184 | MIMAT0004119 | >cfa-miR-369 | MIMAT0006727 |
| >ath-miR418 | MIMAT0001326 | >mdo-miR-187 | MIMAT0004120 | >cfa-miR-410 | MIMAT0006728 |
| >ath-miR419 | MIMAT0001327 | >mdo-miR-181c | MIMAT0004121 | >cfa-miR-126 | MIMAT0006730 |
| >ath-miR420 | MIMAT0001328 | >mdo-miR-186 | MIMAT0004122 | >cfa-miR-212 | MIMAT0006731 |
| >osa-miR413 | MIMAT0001329 | >mdo-miR-193 | MIMAT0004123 | >cfa-miR-132 | MIMAT0006732 |
| >osa-miR414 | MIMAT0001330 | >mdo-miR-196b | MIMAT0004124 | >cfa-miR-22 | MIMAT0006733 |
| >osa-miR415 | MIMAT0001331 | >mdo-miR-199b | MIMAT0004125 | >cfa-miR-144 | MIMAT0006734 |
| >osa-miR416 | MIMAT0001332 | >mdo-miR-203 | MIMAT0004126 | >cfa-miR-193a | MIMAT0006735 |
| >osa-miR417 | MIMAT0001333 | >mdo-miR-204 | MIMAT0004127 | >cfa-miR-142 | MIMAT0006736 |
| >osa-miR418 | MIMAT0001334 | >mdo-miR-206 | MIMAT0004128 | >cfa-miR-10 | MIMAT0006737 |
| >osa-miR419 | MIMAT0001335 | >mdo-miR-208 | MIMAT0004129 | >cfa-miR-152 | MIMAT0006738 |
| >osa-miR420 | MIMAT0001336 | >mdo-miR-214 | MIMAT0004130 | >cfa-miR-338 | MIMAT0006739 |
| >ath-miR426 | MIMAT0001337 | >mdo-miR-216 | MIMAT0004131 | >cfa-miR-1844 | MIMAT0006740 |
| >osa-miR426 | MIMAT0001338 | >mdo-miR-217 | MIMAT0004132 | >cfa-miR-21 | MIMAT0006741 |
| >hsa-miR-422a | MIMAT0001339 | >mdo-miR-218 | MIMAT0004133 | >cfa-miR-423a | MIMAT0006742 |
| >hsa-miR-423-5p | MIMAT0004748 | >mdo-miR-219 | MIMAT0004134 | >cfa-miR-652 | MIMAT0006743 |
| >hsa-miR-423-3p | MIMAT0001340 | >mdo-miR-223 | MIMAT0004135 | >cfa-miR-224 | MIMAT0006744 |
| >hsa-miR-424 | MIMAT0001341 | >mdo-miR-338 | MIMAT0004136 | >cfa-miR-424 | MIMAT0006745 |
| >mmu-miR-425 | MIMAT0004750 | >mdo-miR-365 | MIMAT0004137 | >cfa-miR-503 | MIMAT0006746 |
| >hsa-miR-425 | MIMAT0003393 | >mdo-miR-375 | MIMAT0004138 | >cfa-miR-542 | MIMAT0006747 |
| >xla-miR-427 | MIMAT0001344 | >mdo-miR-383 | MIMAT0004139 | >cfa-miR-450b | MIMAT0006748 |
| >xla-miR-428 | MIMAT0001345 | >mdo-miR-449 | MIMAT0004140 | >cfa-miR-106a | MIMAT0006749 |
| >xla-miR-429 | MIMAT0001346 | >mdo-let-7a | MIMAT0004141 | >cfa-miR-363 | MIMAT0006750 |
| >xla-miR-19b | MIMAT0001347 | >mdo-let-7g | MIMAT0004142 | >cfa-miR-361 | MIMAT0006751 |
| >xla-miR-20 | MIMAT0001348 | >mdo-let-7i | MIMAT0004143 | >cfa-miR-384 | MIMAT0006752 |
| >xla-miR-18 | MIMAT0001349 | >mdo-miR-15a | MIMAT0004144 | >cfa-miR-374a | MIMAT0006753 |
| >xla-miR-133a | MIMAT0001350 | >mdo-miR-16 | MIMAT0004145 | >cfa-miR-374b | MIMAT0006754 |
| >zma-miR156d | MIMAT0001351 | >mdo-miR-183 | MIMAT0004146 | >cfa-miR-421 | MIMAT0006755 |
| >zma-miR156f | MIMAT0001352 | >mdo-miR-96 | MIMAT0004147 | >cfa-miR-98 | MIMAT0006756 |
| >zma-miR156g | MIMAT0001353 | >mdo-miR-212 | MIMAT0004148 | >cfa-miR-221 | MIMAT0006757 |
| >zma-miR156b | MIMAT0001354 | >mdo-miR-132 | MIMAT0004149 | >cfa-miR-532 | MIMAT0006758 |
| >zma-miR156c | MIMAT0001355 | >mdo-miR-200c | MIMAT0004150 | >cfa-miR-500 | MIMAT0006759 |
| >zma-miR156e | MIMAT0001356 | >mdo-miR-141 | MIMAT0004151 | >cfa-miR-660 | MIMAT0006760 |
| >zma-miR156a | MIMAT0001357 | >mdo-miR-191 | MIMAT0004152 | >cfa-miR-502 | MIMAT0006761 |
| >zma-miR156h | MIMAT0001358 | >mdo-miR-425 | MIMAT0004153 | >cfa-miR-676 | MIMAT0006762 |
| >zma-miR156i | MIMAT0001359 | >mdo-miR-181a | MIMAT0004154 | >cfa-let-7j | MIMAT0006763 |
| >zma-miR160a | MIMAT0001360 | >mdo-miR-181b | MIMAT0004155 | >hsa-miR-320d | MIMAT0006764 |
| >zma-miR160c | MIMAT0001361 | >mdo-miR-200b | MIMAT0004156 | >hsa-miR-1825 | MIMAT0006765 |
| >zma-miR160d | MIMAT0001362 | >mdo-miR-200a | MIMAT0004158 | >hsa-miR-1826 | MIMAT0006766 |
| >zma-miR160b | MIMAT0001363 | >mdo-miR-222a | MIMAT0004159 | >hsa-miR-1827 | MIMAT0006767 |
| >zma-miR164a | MIMAT0001364 | >mdo-miR-221 | MIMAT0004160 | >cel-miR-1828 | MIMAT0006768 |
| >zma-miR164d | MIMAT0001365 | >mdo-let-7f | MIMAT0004161 | >cel-miR-1829a | MIMAT0006769 |
| >zma-miR164b | MIMAT0001366 | >mdo-let-7b | MIMAT0004162 | >cel-miR-1829b | MIMAT0006770 |
| >zma-miR164c | MIMAT0000367 | >mdo-miR-29b | MIMAT0004163 | >cel-miR-1829c | MIMAT0006771 |
| >zma-miR169a | MIMAT0001368 | >mdo-miR-29a | MIMAT0004164 | >cel-miR-1830 | MIMAT0006772 |
| >zma-miR169b | MIMAT0001369 | >mdo-miR-17-5p | MIMAT0004165 | >cel-miR-1831 | MIMAT0006773 |
| >zma-miR167a | MIMAT0001370 | >mdo-miR-17-3p | MIMAT0004166 | >cel-miR-1832 | MIMAT0006774 |
| >zma-miR167b | MIMAT0001371 | >mdo-miR-18 | MIMAT0004167 | >cel-miR-1833 | MIMAT0006775 |
| >zma-miR167d | MIMAT0001372 | >mdo-miR-19a | MIMAT0004168 | >cel-miR-1834 | MIMAT0006776 |
| >zma-miR167c | MIMAT0001373 | >mdo-miR-20 | MIMAT0004169 | >gga-miR-216b | MIMAT0007748 |
| >zma-miR160e | MIMAT0001374 | >mdo-miR-19b | MIMAT0004170 | >gga-miR-1845 | MIMAT0007749 |
| >zma-miR166a | MIMAT0001375 | >mdo-miR-92 | MIMAT0004171 | >gga-miR-739 | MIMAT0007750 |
| >zma-miR162 | MIMAT0001376 | >mdo-let-7d | MIMAT0004172 | >gga-miR-214 | MIMAT0007751 |
| >zma-miR166h | MIMAT0001377 | >mdo-miR-23a | MIMAT0004173 | >gga-miR-762 | MIMAT0007752 |
| >zma-miR166e | MIMAT0001378 | >mdo-miR-27a | MIMAT0004174 | >ssc-miR-15a | MIMAT0007753 |
| >zma-miR166i | MIMAT0001379 | >mdo-miR-24 | MIMAT0004175 | >ssc-miR-16 | MIMAT0007754 |
| >zma-miR166f | MIMAT0001380 | >mdo-miR-23b | MIMAT0004176 | >ssc-miR-17 | MIMAT0007755 |
| >zma-miR166g | MIMAT0001381 | >mdo-miR-27b | MIMAT0004177 | >ssc-miR-30b | MIMAT0007756 |
| >zma-miR166b | MIMAT0001382 | >mdo-miR-93 | MIMAT0004178 | >ssc-miR-34a | MIMAT0007757 |
| >zma-miR166c | MIMAT0001383 | >mdo-miR-25 | MIMAT0004179 | >ssc-miR-130a | MIMAT0007758 |
| >zma-miR166d | MIMAT0001384 | >mdo-miR-302b | MIMAT0004180 | >ssc-miR-185 | MIMAT0007759 |
| >zma-miR171a | MIMAT0001385 | >mdo-miR-302c | MIMAT0004181 | >ssc-miR-210 | MIMAT0007761 |
| >zma-miR171b | MIMAT0001386 | >mdo-miR-302a | MIMAT0004182 | >ssc-miR-221 | MIMAT0007762 |
| >zma-miR172a | MIMAT0001387 | >mdo-miR-302d | MIMAT0004183 | >osa-miR531b | MIMAT0007763 |
| >zma-miR172d | MIMAT0001388 | >mdo-miR-367 | MIMAT0004184 | >osa-miR1846d-5p | MIMAT0007764 |
| >zma-miR172b | MIMAT0001389 | >bmo-miR-31 | MIMAT0004213 | >osa-miR1846d-3p | MIMAT0007765 |
| >zma-miR172c | MIMAT0001390 | >bmo-miR-71 | MIMAT0004214 | >osa-miR1847.1 | MIMAT0007766 |
| >sbi-miR166d | MIMAT0001391 | >ath-miR822 | MIMAT0004239 | >osa-miR1847.2 | MIMAT0007767 |
| >sbi-miR166c | MIMAT0001392 | >ath-miR823 | MIMAT0004240 | >osa-miR1848 | MIMAT0007768 |
| >sbi-miR166b | MIMAT0001393 | >ath-miR825 | MIMAT0004241 | >osa-miR1849 | MIMAT0007769 |
| >sbi-miR166a | MIMAT0001394 | >ath-miR826 | MIMAT0004242 | >osa-miR1850 | MIMAT0007770 |
| >sbi-miR172b | MIMAT0001395 | >ath-miR827 | MIMAT0004243 | >osa-miR1851 | MIMAT0007771 |
| >sbi-miR172c | MIMAT0001396 | >ath-miR828 | MIMAT0004244 | >osa-miR1852 | MIMAT0007772 |
| >sbi-miR172a | MIMAT0001397 | >ath-miR829.1 | MIMAT0004245 | >osa-miR1853-5p | MIMAT0007773 |
| >sbi-miR156a | MIMAT0001398 | >ath-miR829.2 | MIMAT0004246 | >osa-miR1853-3p | MIMAT0007774 |
| >sbi-miR156c | MIMAT0001399 | >ath-miR830 | MIMAT0004248 | >osa-miR1854-5p | MIMAT0007775 |
| >sbi-miR156b | MIMAT0001400 | >ath-miR831 | MIMAT0004249 | >osa-miR1854-3p | MIMAT0007776 |
| >sbi-miR160d | MIMAT0001401 | >ath-miR832-5p | MIMAT0004250 | >osa-miR1855 | MIMAT0007777 |
| >sbi-miR160a | MIMAT0001402 | >ath-miR832-3p | MIMAT0004251 | >osa-miR1856 | MIMAT0007778 |
| >sbi-miR160c | MIMAT0001403 | >ath-miR833-5p | MIMAT0004252 | >osa-miR1857-5p | MIMAT0007779 |
| >sbi-miR160b | MIMAT0001404 | >ath-miR833-3p | MIMAT0004253 | >osa-miR1857-3p | MIMAT0007780 |
| >sbi-miR160e | MIMAT0001405 | >ath-miR834 | MIMAT0004254 | >osa-miR1428b | MIMAT0007781 |
| >sbi-miR164 | MIMAT0001406 | >ath-miR835-5p | MIMAT0004255 | >osa-miR1428c | MIMAT0007782 |
| >sbi-miR167a | MIMAT0001407 | >ath-miR835-3p | MIMAT0004256 | >osa-miR1428d | MIMAT0007783 |
| >sbi-miR167b | MIMAT0001408 | >ath-miR836 | MIMAT0004257 | >osa-miR1428e-5p | MIMAT0007784 |
| >sbi-miR169b | MIMAT0001409 | >ath-miR837-5p | MIMAT0004258 | >osa-miR1428e-3p | MIMAT0007785 |
| >sbi-miR169a | MIMAT0001410 | >ath-miR837-3p | MIMAT0004259 | >osa-miR1858a | MIMAT0007786 |
| >gga-miR-20b | MIMAT0001411 | >ath-miR838 | MIMAT0004260 | >osa-miR1858b | MIMAT0007787 |
| >hsa-miR-18b | MIMAT0001412 | >ath-miR839 | MIMAT0004261 | >osa-miR1846a-5p | MIMAT0007788 |
| >hsa-miR-20b | MIMAT0001413 | >ath-miR840 | MIMAT0004262 | >osa-miR1846a-3p | MIMAT0007789 |
| >oar-miR-431 | MIMAT0001414 | >ath-miR841 | MIMAT0004263 | >osa-miR1846b-5p | MIMAT0007790 |
| >oar-miR-127 | MIMAT0001415 | >ath-miR842 | MIMAT0004264 | >osa-miR1846b-3p | MIMAT0007791 |
| >oar-miR-432 | MIMAT0001416 | >ath-miR843 | MIMAT0004265 | >osa-miR1859 | MIMAT0007792 |
| >oar-miR-136 | MIMAT0001417 | >ath-miR844 | MIMAT0004266 | >osa-miR1860-5p | MIMAT0007793 |
| >mmu-miR-431 | MIMAT0001418 | >ath-miR845a | MIMAT0004268 | >osa-miR1860-3p | MIMAT0007794 |
| >mmu-miR-433 | MIMAT0001420 | >ath-miR846 | MIMAT0004269 | >osa-miR1884a | MIMAT0007795 |
| >mmu-miR-434-5p | MIMAT0001421 | >ath-miR848 | MIMAT0004270 | >osa-miR1861a | MIMAT0007796 |
| >mmu-miR-434-3p | MIMAT0001422 | >ath-miR849 | MIMAT0004271 | >osa-miR1861b | MIMAT0007797 |
| >dre-miR-430a | MIMAT0001423 | >ath-miR850 | MIMAT0004272 | >osa-miR1861c | MIMAT0007798 |
| >dre-miR-430b | MIMAT0001424 | >ath-miR851-5p | MIMAT0004273 | >osa-miR1861d | MIMAT0007799 |
| >dre-miR-430c | MIMAT0001425 | >ath-miR851-3p | MIMAT0004274 | >osa-miR1861e | MIMAT0007800 |
| >sbi-miR393 | MIMAT0001426 | >ath-miR852 | MIMAT0004275 | >osa-miR1861f | MIMAT0007801 |
| >sbi-miR394a | MIMAT0001427 | >ath-miR853 | MIMAT0004276 | >osa-miR1861g | MIMAT0007802 |
| >sbi-miR394b | MIMAT0001428 | >ath-miR824 | MIMAT0004277 | >osa-miR1861h | MIMAT0007803 |
| >sbi-miR395b | MIMAT0001429 | >ath-miR847 | MIMAT0004278 | >osa-miR1861i | MIMAT0007804 |
| >sbi-miR395a | MIMAT0001430 | >ath-miR855 | MIMAT0004279 | >osa-miR1861j | MIMAT0007805 |
| >sbi-miR395c | MIMAT0001431 | >ath-miR854a | MIMAT0004280 | >osa-miR1861k | MIMAT0007806 |
| >sbi-miR395d | MIMAT0001432 | >ath-miR854b | MIMAT0004281 | >osa-miR1861l | MIMAT0007807 |
| >sbi-miR395e | MIMAT0001433 | >ath-miR854c | MIMAT0004282 | >osa-miR1861m | MIMAT0007808 |
| >sbi-miR396b | MIMAT0001434 | >ath-miR854d | MIMAT0004283 | >osa-miR1861n | MIMAT0007809 |
| >sbi-miR396a | MIMAT0001435 | >hsa-miR-675 | MIMAT0004284 | >osa-miR1862a | MIMAT0007810 |
| >sbi-miR396c | MIMAT0001436 | >cbr-miR-784 | MIMAT0004285 | >osa-miR1862b | MIMAT0007811 |
| >sbi-miR399a | MIMAT0001437 | >cbr-miR-785 | MIMAT0004286 | >osa-miR1862c | MIMAT0007812 |
| >sbi-miR399c | MIMAT0001438 | >cbr-miR-786 | MIMAT0004287 | >osa-miR1863 | MIMAT0007813 |
| >sbi-miR399d | MIMAT0001439 | >cbr-miR-787 | MIMAT0004288 | >osa-miR1864 | MIMAT0007814 |
| >sbi-miR399e | MIMAT0001440 | >cbr-miR-788 | MIMAT0004289 | >osa-miR1865-5p | MIMAT0007815 |
| >sbi-miR399f | MIMAT0001441 | >cbr-miR-789a | MIMAT0004290 | >osa-miR1865-3p | MIMAT0007816 |
| >sbi-miR399b | MIMAT0001442 | >cbr-miR-789b | MIMAT0004291 | >osa-miR1866 | MIMAT0007817 |
| >sbi-miR399g | MIMAT0001443 | >cbr-miR-790 | MIMAT0004292 | >osa-miR1867 | MIMAT0007818 |
| >sbi-miR156d | MIMAT0001444 | >cbr-miR-791 | MIMAT0004293 | >osa-miR1423b | MIMAT0007819 |
| >sbi-miR164b | MIMAT0001445 | >cbr-miR-792 | MIMAT0004294 | >osa-miR1868 | MIMAT0007820 |
| >sbi-miR166e | MIMAT0001446 | >cbr-miR-235 | MIMAT0004295 | >osa-miR812f | MIMAT0007821 |
| >sbi-miR167d | MIMAT0001447 | >cbr-miR-242 | MIMAT0004296 | >osa-miR1869 | MIMAT0007822 |
| >sbi-miR167f | MIMAT0001448 | >cbr-miR-255 | MIMAT0004297 | >osa-miR1870 | MIMAT0007823 |
| >sbi-miR167g | MIMAT0001449 | >cbr-miR-359 | MIMAT0004298 | >osa-miR1871 | MIMAT0007824 |
| >sbi-miR167e | MIMAT0001450 | >cbr-miR-392 | MIMAT0004299 | >osa-miR1872 | MIMAT0007825 |
| >sbi-miR167c | MIMAT0001451 | >ath-miR856 | MIMAT0004300 | >osa-miR1873 | MIMAT0007826 |
| >sbi-miR168 | MIMAT0001452 | >ath-miR857 | MIMAT0004301 | >osa-miR1874-5p | MIMAT0007827 |
| >sbi-miR169c | MIMAT0001453 | >ath-miR858 | MIMAT0004302 | >osa-miR1874-3p | MIMAT0007828 |
| >sbi-miR169d | MIMAT0001454 | >ath-miR859 | MIMAT0004303 | >osa-miR1875 | MIMAT0007829 |
| >sbi-miR169e | MIMAT0001455 | >ath-miR860 | MIMAT0004304 | >osa-miR1862d | MIMAT0007830 |
| >sbi-miR169f | MIMAT0001456 | >ath-miR861-5p | MIMAT0004305 | >osa-miR1876 | MIMAT0007831 |
| >sbi-miR169g | MIMAT0001457 | >ath-miR861-3p | MIMAT0004306 | >osa-miR1884b | MIMAT0007832 |
| >sbi-miR169h | MIMAT0001458 | >ath-miR862-5p | MIMAT0004307 | >osa-miR1862e | MIMAT0007833 |
| >sbi-miR169i | MIMAT0001459 | >ath-miR862-3p | MIMAT0004308 | >osa-miR1877 | MIMAT0007834 |
| >sbi-miR171b | MIMAT0001460 | >ath-miR863-5p | MIMAT0004309 | >osa-miR1878 | MIMAT0007835 |
| >sbi-miR171d | MIMAT0001461 | >ath-miR863-3p | MIMAT0004310 | >osa-miR1879 | MIMAT0007836 |
| >sbi-miR171a | MIMAT0001462 | >ath-miR864-5p | MIMAT0004311 | >osa-miR1880 | MIMAT0007837 |
| >sbi-miR171c | MIMAT0001463 | >ath-miR864-3p | MIMAT0004312 | >osa-miR1881 | MIMAT0007838 |
| >sbi-miR172e | MIMAT0001464 | >ath-miR865-5p | MIMAT0004313 | >osa-miR1882a | MIMAT0007839 |
| >sbi-miR166f | MIMAT0001465 | >ath-miR865-3p | MIMAT0004314 | >osa-miR1882b | MIMAT0007840 |
| >sbi-miR171e | MIMAT0001466 | >ath-miR866-5p | MIMAT0004315 | >osa-miR1882c | MIMAT0007841 |
| >sbi-miR172d | MIMAT0001467 | >ath-miR866-3p | MIMAT0004316 | >osa-miR1882d | MIMAT0007842 |
| >sbi-miR159 | MIMAT0001468 | >ath-miR845b | MIMAT0004317 | >osa-miR1882e | MIMAT0007843 |
| >sbi-miR319 | MIMAT0001469 | >ath-miR867 | MIMAT0004318 | >osa-miR1882f | MIMAT0007844 |
| >ame-bantam | MIMAT0001470 | >ath-miR868 | MIMAT0004319 | >osa-miR1882g | MIMAT0007845 |
| >ame-miR-1 | MIMAT0001471 | >ath-miR869.1 | MIMAT0004320 | >osa-miR1882h | MIMAT0007846 |
| >ame-miR-12 | MIMAT0001472 | >ath-miR869.2 | MIMAT0004321 | >osa-miR812g | MIMAT0007847 |
| >ame-miR-124 | MIMAT0001473 | >ath-miR870 | MIMAT0004322 | >osa-miR812h | MIMAT0007848 |
| >ame-miR-125 | MIMAT0001474 | >mmu-miR-181d | MIMAT0004324 | >osa-miR812i | MIMAT0007849 |
| >ame-miR-133 | MIMAT0001475 | >bta-let-7b | MIMAT0004331 | >osa-miR812j | MIMAT0007850 |
| >ame-miR-184 | MIMAT0001476 | >bta-let-7c | MIMAT0004332 | >osa-miR1883a | MIMAT0007851 |
| >ame-miR-210 | MIMAT0001477 | >bta-let-7e | MIMAT0004333 | >osa-miR1883b | MIMAT0007852 |
| >ame-miR-219 | MIMAT0001478 | >bta-miR-15a | MIMAT0004334 | >ath-miR1886 | MIMAT0007853 |
| >ame-miR-263 | MIMAT0001479 | >bta-miR-195 | MIMAT0004335 | >ath-miR1887 | MIMAT0007854 |
| >ame-miR-276 | MIMAT0001480 | >bta-miR-19a | MIMAT0004336 | >ath-miR1888 | MIMAT0007855 |
| >ame-miR-277 | MIMAT0001481 | >bta-miR-19b | MIMAT0004337 | >aga-miR-11 | MIMAT0007856 |
| >ame-miR-278 | MIMAT0001482 | >bta-miR-204 | MIMAT0004338 | >aga-miR-981 | MIMAT0007857 |
| >ame-miR-281 | MIMAT0001483 | >bta-miR-331 | MIMAT0004339 | >aga-miR-87 | MIMAT0007858 |
| >ame-miR-282 | MIMAT0001484 | >bta-miR-34a | MIMAT0004340 | >aga-miR-1889 | MIMAT0007859 |
| >ame-miR-2 | MIMAT0001485 | >bta-miR-365 | MIMAT0004341 | >aga-miR-375 | MIMAT0007860 |
| >ame-miR-305 | MIMAT0001486 | >bta-miR-374a | MIMAT0004342 | >aga-miR-1890 | MIMAT0007861 |
| >ame-miR-315 | MIMAT0001487 | >bta-miR-497 | MIMAT0004343 | >aga-miR-1891 | MIMAT0007862 |
| >ame-miR-317 | MIMAT0001488 | >bta-miR-660 | MIMAT0004344 | >mmu-miR-1902 | MIMAT0007863 |
| >ame-miR-7 | MIMAT0001489 | >bta-miR-99b | MIMAT0004345 | >mmu-miR-1897-5p | MIMAT0007864 |
| >ame-miR-8 | MIMAT0001490 | >mmu-miR-743b-5p | MIMAT0004839 | >mmu-miR-1897-3p | MIMAT0007865 |
| >ame-miR-9a | MIMAT0001491 | >mmu-miR-743b-3p | MIMAT0004840 | >mmu-miR-1905 | MIMAT0007866 |
| >ame-miR-9b | MIMAT0001492 | >mmu-miR-871 | MIMAT0004841 | >mmu-miR-1895 | MIMAT0007867 |
| >ame-miR-iab-4 | MIMAT0001493 | >mmu-miR-879 | MIMAT0004842 | >mmu-miR-1903 | MIMAT0007868 |
| >aga-bantam | MIMAT0001494 | >mmu-miR-880 | MIMAT0004844 | >mmu-miR-1899 | MIMAT0007869 |
| >aga-let-7 | MIMAT0001495 | >mmu-miR-881 | MIMAT0004846 | >mmu-miR-1900 | MIMAT0007870 |
| >aga-miR-1 | MIMAT0001496 | >mmu-miR-882 | MIMAT0004847 | >mmu-miR-1892 | MIMAT0007871 |
| >aga-miR-10 | MIMAT0001497 | >mmu-miR-883a-5p | MIMAT0004848 | >mmu-miR-1906 | MIMAT0007872 |
| >aga-miR-100 | MIMAT0001498 | >mmu-miR-883a-3p | MIMAT0004849 | >mmu-miR-1896 | MIMAT0007873 |
| >aga-miR-124 | MIMAT0001499 | >mmu-miR-883b-5p | MIMAT0004850 | >mmu-miR-1904 | MIMAT0007874 |
| >aga-miR-125 | MIMAT0001500 | >mmu-miR-883b-3p | MIMAT0004851 | >mmu-miR-1898 | MIMAT0007875 |
| >aga-miR-133 | MIMAT0001501 | >mmu-miR-190b | MIMAT0004852 | >mmu-miR-1907 | MIMAT0007876 |
| >aga-miR-13b | MIMAT0001502 | >mmu-miR-874 | MIMAT0004853 | >mmu-miR-1894-5p | MIMAT0007877 |
| >aga-miR-14 | MIMAT0001503 | >mmu-miR-876-5p | MIMAT0004854 | >mmu-miR-1894-3p | MIMAT0007878 |
| >aga-miR-184 | MIMAT0001504 | >mmu-miR-876-3p | MIMAT0004855 | >mmu-miR-1893 | MIMAT0007879 |
| >aga-miR-210 | MIMAT0001505 | >mmu-miR-105 | MIMAT0004856 | >mmu-miR-1901 | MIMAT0007880 |
| >aga-miR-219 | MIMAT0001506 | >mmu-miR-147 | MIMAT0004857 | >hsa-miR-1908 | MIMAT0007881 |
| >aga-miR-263 | MIMAT0001507 | >mmu-miR-18b | MIMAT0004858 | >hsa-miR-1909 | MIMAT0007883 |
| >aga-miR-275 | MIMAT0001508 | >mmu-miR-193b | MIMAT0004859 | >hsa-miR-1910 | MIMAT0007884 |
| >aga-miR-276-5p | MIMAT0001509 | >mmu-miR-197 | MIMAT0004860 | >hsa-miR-1911 | MIMAT0007885 |
| >aga-miR-276-3p | MIMAT0006782 | >mmu-miR-220 | MIMAT0004863 | >hsa-miR-1912 | MIMAT0007887 |
| >aga-miR-277 | MIMAT0001510 | >mmu-miR-297c | MIMAT0004865 | >hsa-miR-1913 | MIMAT0007888 |
| >aga-miR-278 | MIMAT0001511 | >mmu-miR-327 | MIMAT0004867 | >hsa-miR-1914 | MIMAT0007889 |
| >aga-miR-279 | MIMAT0001512 | >mmu-miR-343 | MIMAT0004868 | >hsa-miR-1915 | MIMAT0007892 |
| >aga-miR-281 | MIMAT0001513 | >mmu-miR-421 | MIMAT0004869 | >mdv1-miR-M31 | MIMAT0007893 |
| >aga-miR-282 | MIMAT0001514 | >mmu-miR-453 | MIMAT0004870 | >bmo-miR-2a | MIMAT0007894 |
| >aga-miR-283 | MIMAT0001515 | >mmu-miR-465b-5p | MIMAT0004871 | >bmo-miR-2b | MIMAT0007895 |
| >aga-miR-2 | MIMAT0001516 | >mmu-miR-465b-3p | MIMAT0004872 | >bmo-miR-13a | MIMAT0007897 |
| >aga-miR-305 | MIMAT0001518 | >mmu-miR-465c-5p | MIMAT0004873 | >bmo-miR-13b | MIMAT0007898 |
| >aga-miR-307 | MIMAT0001519 | >mmu-miR-465c-3p | MIMAT0004874 | >bmo-miR-79 | MIMAT0007899 |
| >aga-miR-308 | MIMAT0001520 | >mmu-miR-466b-5p | MIMAT0004875 | >bmo-miR-87 | MIMAT0007900 |
| >aga-miR-315 | MIMAT0001521 | >mmu-miR-466b-3p | MIMAT0004876 | >bmo-miR-133 | MIMAT0007901 |
| >aga-miR-317 | MIMAT0001522 | >mmu-miR-466b-3-3p | MIMAT0005453 | >bmo-miR-184 | MIMAT0007902 |
| >aga-miR-7 | MIMAT0001523 | >mmu-miR-466c-5p | MIMAT0004877 | >bmo-miR-210 | MIMAT0007903 |
| >aga-miR-79 | MIMAT0001524 | >mmu-miR-466c-3p | MIMAT0004878 | >bmo-miR-281 | MIMAT0007905 |
| >aga-miR-8 | MIMAT0001525 | >mmu-miR-466e-5p | MIMAT0004879 | >bmo-miR-317 | MIMAT0007906 |
| >aga-miR-9a | MIMAT0001526 | >mmu-miR-466e-3p | MIMAT0004880 | >bmo-bantam | MIMAT0007907 |
| >aga-miR-92a | MIMAT0001527 | >mmu-miR-466f-5p | MIMAT0004881 | >sly-miR1916 | MIMAT0007908 |
| >aga-miR-92b | MIMAT0001528 | >mmu-miR-466f-3p | MIMAT0004882 | >sly-miR1917 | MIMAT0007909 |
| >aga-miR-9b | MIMAT0001529 | >mmu-miR-466g | MIMAT0004883 | >sly-miR1918 | MIMAT0007910 |
| >aga-miR-9c | MIMAT0001530 | >mmu-miR-466h | MIMAT0004884 | >sly-miR1919a | MIMAT0007911 |
| >aga-miR-iab-4 | MIMAT0001531 | >mmu-miR-467c | MIMAT0004885 | >sly-miR1919b | MIMAT0007912 |
| >hsa-miR-448 | MIMAT0001532 | >mmu-miR-467d | MIMAT0004886 | >sly-miR1919c | MIMAT0007913 |
| >mmu-miR-448 | MIMAT0001533 | >mmu-miR-493 | MIMAT0004888 | >sly-miR160a | MIMAT0007914 |
| >rno-miR-448 | MIMAT0001534 | >mmu-miR-504 | MIMAT0004889 | >sly-miR166a | MIMAT0007915 |
| >cfa-miR-448 | MIMAT0001535 | >mmu-miR-509-5p | MIMAT0004890 | >sly-miR166b | MIMAT0007916 |
| >hsa-miR-429 | MIMAT0001536 | >mmu-miR-509-3p | MIMAT0004891 | >sly-miR167 | MIMAT0007917 |
| >mmu-miR-429 | MIMAT0001537 | >mmu-miR-568 | MIMAT0004892 | >sly-miR169a | MIMAT0007918 |
| >rno-miR-429 | MIMAT0001538 | >mmu-miR-574-5p | MIMAT0004893 | >sly-miR169b | MIMAT0007919 |
| >cfa-miR-429 | MIMAT0001539 | >mmu-miR-574-3p | MIMAT0004894 | >sly-miR169c | MIMAT0007920 |
| >cfa-miR-365 | MIMAT0001540 | >mmu-miR-590-5p | MIMAT0004895 | >sly-miR169d | MIMAT0007921 |
| >hsa-miR-449a | MIMAT0001541 | >mmu-miR-590-3p | MIMAT0004896 | >sly-miR171a | MIMAT0007922 |
| >mmu-miR-449a | MIMAT0001542 | >mmu-miR-654-5p | MIMAT0004897 | >sly-miR171b | MIMAT0007923 |
| >rno-miR-449a | MIMAT0001543 | >mmu-miR-654-3p | MIMAT0004898 | >sly-miR171c | MIMAT0007924 |
| >cfa-miR-449 | MIMAT0001544 | >mmu-miR-92b | MIMAT0004899 | >sly-miR171d | MIMAT0007925 |
| >hsa-miR-450a | MIMAT0001545 | >hsa-miR-298 | MIMAT0004901 | >sly-miR395a | MIMAT0007926 |
| >mmu-miR-450a-5p | MIMAT0001546 | >hsa-miR-891a | MIMAT0004902 | >sly-miR395b | MIMAT0007927 |
| >rno-miR-450a | MIMAT0001547 | >hsa-miR-300 | MIMAT0004903 | >sly-miR397 | MIMAT0007928 |
| >cfa-miR-450a | MIMAT0001548 | >hsa-miR-886-5p | MIMAT0004905 | >bmo-miR-1920 | MIMAT0007929 |
| >rno-miR-365 | MIMAT0001549 | >hsa-miR-886-3p | MIMAT0004906 | >bmo-miR-1921 | MIMAT0007930 |
| >mghv-miR-M1-1 | MIMAT0001564 | >hsa-miR-892a | MIMAT0004907 | >bmo-miR-1922 | MIMAT0007931 |
| >mghv-miR-M1-2 | MIMAT0001565 | >hsa-miR-220b | MIMAT0004908 | >bmo-miR-1923 | MIMAT0007932 |
| >mghv-miR-M1-3 | MIMAT0001566 | >hsa-miR-450b-5p | MIMAT0004909 | >bmo-miR-1926 | MIMAT0007933 |
| >mghv-miR-M1-4 | MIMAT0001567 | >hsa-miR-450b-3p | MIMAT0004910 | >bmo-miR-1924 | MIMAT0007934 |
| >mghv-miR-M1-5 | MIMAT0001568 | >hsa-miR-874 | MIMAT0004911 | >bmo-miR-1925 | MIMAT0007935 |
| >mghv-miR-M1-6 | MIMAT0001569 | >hsa-miR-890 | MIMAT0004912 | >ptr-let-7a | MIMAT0007936 |
| >mghv-miR-M1-7-5p | MIMAT0001570 | >hsa-miR-891b | MIMAT0004913 | >ptr-let-7b | MIMAT0007937 |
| >mghv-miR-M1-7-3p | MIMAT0001571 | >hsa-miR-220c | MIMAT0004915 | >ptr-let-7c | MIMAT0007938 |
| >mghv-miR-M1-8 | MIMAT0001572 | >hsa-miR-888 | MIMAT0004916 | >ptr-let-7d | MIMAT0007939 |
| >mghv-miR-M1-9 | MIMAT0001573 | >hsa-miR-892b | MIMAT0004918 | >ptr-let-7e | MIMAT0007940 |
| >hcmv-miR-UL22A | MIMAT0001574 | >hsa-miR-541 | MIMAT0004920 | >ptr-let-7f | MIMAT0007941 |
| >hcmv-miR-UL36 | MIMAT0001576 | >hsa-miR-889 | MIMAT0004921 | >ptr-let-7g | MIMAT0007942 |
| >hcmv-miR-UL112 | MIMAT0001577 | >hsa-miR-875-5p | MIMAT0004922 | >ptr-let-7i | MIMAT0007943 |
| >hcmv-miR-UL148D | MIMAT0001578 | >hsa-miR-875-3p | MIMAT0004923 | >ptr-miR-10a | MIMAT0007944 |
| >hcmv-miR-US5-1 | MIMAT0001579 | >hsa-miR-876-5p | MIMAT0004924 | >ptr-miR-10b | MIMAT0007945 |
| >hcmv-miR-US5-2 | MIMAT0001580 | >hsa-miR-876-3p | MIMAT0004925 | >ptr-miR-1 | MIMAT0007946 |
| >hcmv-miR-US25-1 | MIMAT0001581 | >hsa-miR-708 | MIMAT0004926 | >ptr-miR-1178 | MIMAT0007947 |
| >hcmv-miR-US25-2-5p | MIMAT0001582 | >hsa-miR-147b | MIMAT0004928 | >ptr-miR-1179 | MIMAT0007948 |
| >hcmv-miR-US25-2-3p | MIMAT0001583 | >hsa-miR-190b | MIMAT0004929 | >ptr-miR-1181 | MIMAT0007949 |
| >hcmv-miR-US33-5p | MIMAT0001584 | >mmu-miR-466d-5p | MIMAT0004930 | >ptr-miR-1182 | MIMAT0007950 |
| >hcmv-miR-US33-3p | MIMAT0004756 | >mmu-miR-466d-3p | MIMAT0004931 | >ptr-miR-1183 | MIMAT0007951 |
| >osa-miR435 | MIMAT0001585 | >mmu-miR-449b | MIMAT0005447 | >ptr-miR-1184 | MIMAT0007952 |
| >osa-miR437 | MIMAT0001586 | >mmu-miR-878-5p | MIMAT0004932 | >ptr-miR-1185 | MIMAT0007953 |
| >osa-miR438 | MIMAT0001587 | >mmu-miR-878-3p | MIMAT0004933 | >ptr-miR-1197 | MIMAT0007954 |
| >osa-miR390 | MIMAT0001588 | >mmu-miR-872 | MIMAT0004934 | >ptr-miR-1201 | MIMAT0007955 |
| >osa-miR439a | MIMAT0001589 | >mmu-miR-873 | MIMAT0004936 | >ptr-miR-1202 | MIMAT0007956 |
| >osa-miR439b | MIMAT0001590 | >mmu-miR-875-5p | MIMAT0004937 | >ptr-miR-1203 | MIMAT0007957 |
| >osa-miR439c | MIMAT0001591 | >mmu-miR-875-3p | MIMAT0004938 | >ptr-miR-1204 | MIMAT0007958 |
| >osa-miR439d | MIMAT0001592 | >mmu-miR-208b | MIMAT0004939 | >ptr-miR-1205 | MIMAT0007959 |
| >osa-miR439e | MIMAT0001593 | >mmu-miR-877 | MIMAT0004861 | >ptr-miR-1206 | MIMAT0007960 |
| >osa-miR439f | MIMAT0001594 | >mmu-miR-511 | MIMAT0004940 | >ptr-miR-1207 | MIMAT0007961 |
| >osa-miR439g | MIMAT0001595 | >mmu-miR-544 | MIMAT0004941 | >ptr-miR-1208 | MIMAT0007962 |
| >osa-miR439h | MIMAT0001596 | >mmu-miR-598 | MIMAT0004942 | >ptr-miR-122 | MIMAT0007963 |
| >osa-miR439i | MIMAT0001597 | >mmu-miR-653 | MIMAT0004943 | >ptr-miR-1225 | MIMAT0007964 |
| >osa-miR439j | MIMAT0001598 | >hsa-miR-744 | MIMAT0004945 | >ptr-miR-1227 | MIMAT0007965 |
| >osa-miR440 | MIMAT0001599 | >hsa-miR-885-5p | MIMAT0004947 | >ptr-miR-1233 | MIMAT0007966 |
| >osa-miR396d | MIMAT0001600 | >hsa-miR-885-3p | MIMAT0004948 | >ptr-miR-1234 | MIMAT0007967 |
| >osa-miR396e | MIMAT0001601 | >hsa-miR-877 | MIMAT0004949 | >ptr-miR-1236 | MIMAT0007968 |
| >osa-miR441a | MIMAT0001602 | >hsa-miR-887 | MIMAT0004951 | >ptr-miR-1237 | MIMAT0007969 |
| >osa-miR441b | MIMAT0001603 | >hsa-miR-665 | MIMAT0004952 | >ptr-miR-1244 | MIMAT0007970 |
| >osa-miR441c | MIMAT0001604 | >hsa-miR-873 | MIMAT0004953 | >ptr-miR-1245 | MIMAT0007971 |
| >osa-miR442 | MIMAT0001605 | >hsa-miR-543 | MIMAT0004954 | >ptr-miR-1246 | MIMAT0007972 |
| >osa-miR443 | MIMAT0001606 | >hsa-miR-374b | MIMAT0004955 | >ptr-miR-1247 | MIMAT0007973 |
| >osa-miR445a | MIMAT0001607 | >hsa-miR-760 | MIMAT0004957 | >ptr-miR-1248 | MIMAT0007974 |
| >osa-miR445b | MIMAT0001608 | >hsa-miR-301b | MIMAT0004958 | >ptr-miR-1249 | MIMAT0007975 |
| >osa-miR445c | MIMAT0001609 | >hsa-miR-216b | MIMAT0004959 | >ptr-miR-1250 | MIMAT0007976 |
| >osa-miR445d | MIMAT0001610 | >hsa-miR-208b | MIMAT0004960 | >ptr-miR-1251 | MIMAT0007977 |
| >osa-miR445e | MIMAT0001611 | >ghr-miR156a | MIMAT0005806 | >ptr-miR-1253 | MIMAT0007978 |
| >osa-miR445f | MIMAT0001612 | >ghr-miR156b | MIMAT0005807 | >ptr-miR-1254 | MIMAT0007979 |
| >osa-miR445g | MIMAT0001613 | >ghr-miR156c | MIMAT0005808 | >ptr-miR-1255b | MIMAT0007980 |
| >osa-miR445h | MIMAT0001614 | >ghr-miR156d | MIMAT0005809 | >ptr-miR-1256 | MIMAT0007981 |
| >osa-miR445i | MIMAT0001615 | >gra-miR157a | MIMAT0005810 | >ptr-miR-1258 | MIMAT0007982 |
| >osa-miR446 | MIMAT0001616 | >gra-miR157b | MIMAT0005811 | >ptr-miR-1259 | MIMAT0007983 |
| >osa-miR444a.2 | MIMAT0005790 | >ghr-miR162a | MIMAT0005812 | >ptr-miR-125a | MIMAT0007984 |
| >osa-miR444a.1 | MIMAT0001617 | >ghr-miR166b | MIMAT0005813 | >ptr-miR-126 | MIMAT0007985 |
| >dre-miR-429 | MIMAT0001624 | >ghb-miR169a | MIMAT0005814 | >ptr-miR-1262 | MIMAT0007986 |
| >hsa-miR-431 | MIMAT0001625 | >ghr-miR390a | MIMAT0005815 | >ptr-miR-1263 | MIMAT0007987 |
| >rno-miR-431 | MIMAT0001626 | >ghr-miR390b | MIMAT0005816 | >ptr-miR-1264 | MIMAT0007988 |
| >hsa-miR-433 | MIMAT0001627 | >ghr-miR390c | MIMAT0005817 | >ptr-miR-1265 | MIMAT0007989 |
| >rno-miR-433 | MIMAT0001628 | >ghr-miR396a | MIMAT0005818 | >ptr-miR-1266 | MIMAT0007990 |
| >hsa-miR-329 | MIMAT0001629 | >ghr-miR396b | MIMAT0005819 | >ptr-miR-1267 | MIMAT0007991 |
| >hsa-miR-453 | MIMAT0001630 | >ghr-miR399a | MIMAT0005820 | >ptr-miR-1271 | MIMAT0007992 |
| >hsa-miR-451 | MIMAT0001631 | >ghr-miR399b | MIMAT0005821 | >ptr-miR-1272 | MIMAT0007993 |
| >mmu-miR-451 | MIMAT0001632 | >ppt-miR156c | MIMAT0004346 | >ptr-miR-1273 | MIMAT0007994 |
| >rno-miR-451 | MIMAT0001633 | >ppt-miR160a | MIMAT0004347 | >ptr-miR-1274b | MIMAT0007995 |
| >dre-miR-451 | MIMAT0001634 | >ppt-miR160b | MIMAT0004348 | >ptr-miR-1275 | MIMAT0007996 |
| >hsa-miR-452 | MIMAT0001635 | >ppt-miR160c | MIMAT0004349 | >ptr-miR-1276 | MIMAT0007997 |
| >mmu-miR-452 | MIMAT0001637 | >ppt-miR160d | MIMAT0004350 | >ptr-miR-1278 | MIMAT0007998 |
| >hsa-miR-409-5p | MIMAT0001638 | >ppt-miR166b | MIMAT0004351 | >ptr-miR-1280 | MIMAT0007999 |
| >hsa-miR-409-3p | MIMAT0001639 | >ppt-miR166a | MIMAT0004352 | >ptr-miR-1281 | MIMAT0008000 |
| >mtr-miR162 | MIMAT0001640 | >ppt-miR167 | MIMAT0004353 | >ptr-miR-1282 | MIMAT0008001 |
| >mtr-miR160 | MIMAT0001641 | >ppt-miR319e | MIMAT0004354 | >ptr-miR-1283 | MIMAT0008002 |
| >mtr-miR166 | MIMAT0001642 | >ppt-miR395 | MIMAT0004355 | >ptr-miR-1284 | MIMAT0008003 |
| >mtr-miR169a | MIMAT0001643 | >ppt-miR408 | MIMAT0004356 | >ptr-miR-1285 | MIMAT0008004 |
| >mtr-miR169b | MIMAT0001644 | >ppt-miR414 | MIMAT0004357 | >ptr-miR-1286 | MIMAT0008005 |
| >mtr-miR399b | MIMAT0001645 | >ppt-miR419 | MIMAT0004358 | >ptr-miR-1288 | MIMAT0008006 |
| >mtr-miR399d | MIMAT0001646 | >ppt-miR477c | MIMAT0004359 | >ptr-miR-1289 | MIMAT0008007 |
| >mtr-miR393 | MIMAT0001647 | >ppt-miR477a-5p | MIMAT0004360 | >ptr-miR-1290 | MIMAT0008008 |
| >mtr-miR395a | MIMAT0001648 | >ppt-miR477a-3p | MIMAT0003899 | >ptr-miR-1291 | MIMAT0008009 |
| >mtr-miR395b | MIMAT0001649 | >ppt-miR477f | MIMAT0004361 | >ptr-miR-129 | MIMAT0008010 |
| >mtr-miR399c | MIMAT0001650 | >ppt-miR477g-5p | MIMAT0004362 | >ptr-miR-1292 | MIMAT0008011 |
| >mtr-miR399a | MIMAT0001651 | >ppt-miR477g-3p | MIMAT0003902 | >ptr-miR-1293 | MIMAT0008012 |
| >mtr-miR399e | MIMAT0001652 | >ppt-miR534b | MIMAT0004363 | >ptr-miR-1294 | MIMAT0008013 |
| >mtr-miR319 | MIMAT0001653 | >ppt-miR893 | MIMAT0004364 | >ptr-miR-1295 | MIMAT0008014 |
| >mtr-miR156 | MIMAT0001654 | >ppt-miR894 | MIMAT0004365 | >ptr-miR-1296 | MIMAT0008015 |
| >mtr-miR171 | MIMAT0001655 | >ppt-miR895 | MIMAT0004366 | >ptr-miR-1297 | MIMAT0008016 |
| >sof-miR156 | MIMAT0001656 | >ppt-miR896 | MIMAT0004367 | >ptr-miR-1298 | MIMAT0008017 |
| >sof-miR396 | MIMAT0001657 | >ppt-miR897 | MIMAT0004368 | >ptr-miR-1299 | MIMAT0008018 |
| >sof-miR159a | MIMAT0001658 | >ppt-miR899 | MIMAT0004369 | >ptr-miR-1300b | MIMAT0008019 |
| >sof-miR159b | MIMAT0001659 | >ppt-miR900-5p | MIMAT0004961 | >ptr-miR-1300a | MIMAT0008020 |
| >sof-miR159d | MIMAT0001660 | >ppt-miR900-3p | MIMAT0004370 | >ptr-miR-1302 | MIMAT0008021 |
| >sof-miR159e | MIMAT0001661 | >ppt-miR901 | MIMAT0004371 | >ptr-miR-1303 | MIMAT0008022 |
| >sof-miR159c | MIMAT0001662 | >ppt-miR902a-5p | MIMAT0004962 | >ptr-miR-1306 | MIMAT0008023 |
| >sof-miR167a | MIMAT0001663 | >ppt-miR902a-3p | MIMAT0004372 | >ptr-miR-1307 | MIMAT0008024 |
| >sof-miR167b | MIMAT0001664 | >ppt-miR902b-5p | MIMAT0004963 | >ptr-miR-130a | MIMAT0008025 |
| >sof-miR168a | MIMAT0001665 | >ppt-miR902b-3p | MIMAT0004373 | >ptr-miR-130b | MIMAT0008026 |
| >sof-miR168b | MIMAT0001666 | >ppt-miR536c | MIMAT0004374 | >ptr-miR-1322 | MIMAT0008027 |
| >sof-miR408a | MIMAT0001667 | >ppt-miR171a | MIMAT0004375 | >ptr-miR-1323 | MIMAT0008028 |
| >sof-miR408b | MIMAT0001668 | >ppt-miR171b | MIMAT0004376 | >ptr-miR-1324 | MIMAT0008029 |
| >sof-miR408c | MIMAT0001669 | >ppt-miR903 | MIMAT0004377 | >ptr-miR-133b | MIMAT0008030 |
| >sof-miR408d | MIMAT0001670 | >ppt-miR536b | MIMAT0004378 | >ptr-miR-134 | MIMAT0008031 |
| >sof-miR408e | MIMAT0001671 | >ppt-miR904a | MIMAT0004379 | >ptr-miR-135b | MIMAT0008032 |
| >gma-miR156d | MIMAT0001672 | >ppt-miR904b | MIMAT0004380 | >ptr-miR-137 | MIMAT0008033 |
| >gma-miR156e | MIMAT0001673 | >ppt-miR898b | MIMAT0004381 | >ptr-miR-138 | MIMAT0008034 |
| >gma-miR156c | MIMAT0001674 | >ppt-miR898a | MIMAT0004382 | >ptr-miR-139 | MIMAT0008035 |
| >gma-miR159a | MIMAT0001675 | >ppt-miR156b | MIMAT0004384 | >ptr-miR-141 | MIMAT0008036 |
| >gma-miR160 | MIMAT0001676 | >cre-miR918 | MIMAT0004385 | >ptr-miR-142 | MIMAT0008037 |
| >gma-miR166a | MIMAT0001677 | >cre-miR905 | MIMAT0004386 | >ptr-miR-146a | MIMAT0008038 |
| >gma-miR166b | MIMAT0001678 | >cre-miR906-5p | MIMAT0004387 | >ptr-miR-146b | MIMAT0008039 |
| >gma-miR167a | MIMAT0001679 | >cre-miR906-3p | MIMAT0004388 | >ptr-miR-147b | MIMAT0008040 |
| >gma-miR167b | MIMAT0001680 | >cre-miR907 | MIMAT0004389 | >ptr-miR-148a | MIMAT0008041 |
| >gma-miR168 | MIMAT0001681 | >cre-miR908.1 | MIMAT0004965 | >ptr-miR-148b | MIMAT0008042 |
| >gma-miR172a | MIMAT0001682 | >cre-miR908.2 | MIMAT0004390 | >ptr-miR-149 | MIMAT0008043 |
| >gma-miR172b | MIMAT0001683 | >cre-miR908.3 | MIMAT0004966 | >ptr-miR-150 | MIMAT0008044 |
| >gma-miR319a | MIMAT0001684 | >cre-miR909.2 | MIMAT0004391 | >ptr-miR-151 | MIMAT0008045 |
| >gma-miR319b | MIMAT0001685 | >cre-miR909.1 | MIMAT0004967 | >ptr-miR-152 | MIMAT0008046 |
| >gma-miR156a | MIMAT0001686 | >cre-miR909.3 | MIMAT0004392 | >ptr-miR-153 | MIMAT0008047 |
| >gma-miR396a | MIMAT0001687 | >cre-miR910 | MIMAT0004393 | >ptr-miR-155 | MIMAT0008048 |
| >gma-miR396b | MIMAT0001688 | >cre-miR911 | MIMAT0004394 | >ptr-miR-181d | MIMAT0008049 |
| >gma-miR398a | MIMAT0001689 | >cre-miR912 | MIMAT0004395 | >ptr-miR-182 | MIMAT0008050 |
| >gma-miR398b | MIMAT0001690 | >cre-miR913 | MIMAT0004968 | >ptr-miR-1825 | MIMAT0008051 |
| >gma-miR319c | MIMAT0001691 | >cre-miR914 | MIMAT0004397 | >ptr-miR-1827 | MIMAT0008052 |
| >gma-miR156b | MIMAT0001692 | >cre-miR915 | MIMAT0004398 | >ptr-miR-185 | MIMAT0008053 |
| >gma-miR169a | MIMAT0001693 | >cre-miR919.2 | MIMAT0004399 | >ptr-miR-18b | MIMAT0008054 |
| >zma-miR171d | MIMAT0001694 | >cre-miR919.1 | MIMAT0004969 | >ptr-miR-190b | MIMAT0008055 |
| >zma-miR171f | MIMAT0001695 | >cre-miR917 | MIMAT0004404 | >ptr-miR-191 | MIMAT0008056 |
| >zma-miR394a | MIMAT0001696 | >cre-miR916 | MIMAT0004405 | >ptr-miR-192 | MIMAT0008057 |
| >zma-miR394b | MIMAT0001697 | >hsa-miR-920 | MIMAT0004970 | >ptr-miR-193a | MIMAT0008058 |
| >zma-miR395b | MIMAT0001698 | >hsa-miR-921 | MIMAT0004971 | >ptr-miR-193b | MIMAT0008059 |
| >zma-miR395c | MIMAT0001700 | >hsa-miR-922 | MIMAT0004972 | >ptr-miR-195 | MIMAT0008060 |
| >zma-miR395a | MIMAT0001701 | >hsa-miR-923 | MIMAT0004973 | >ptr-miR-196b | MIMAT0008061 |
| >zma-miR396b | MIMAT0001702 | >hsa-miR-924 | MIMAT0004974 | >ptr-miR-199a-3p | MIMAT0009192 |
| >zma-miR396a | MIMAT0001703 | >hsa-miR-509-3-5p | MIMAT0004975 | >ptr-miR-199b | MIMAT0008062 |
| >zma-miR399a | MIMAT0001704 | >rrv-miR-rR1-1 | MIMAT0004406 | >ptr-miR-200a | MIMAT0008063 |
| >zma-miR399c | MIMAT0001705 | >rrv-miR-rR1-2 | MIMAT0004408 | >ptr-miR-202 | MIMAT0008064 |
| >zma-miR399b | MIMAT0001706 | >rrv-miR-rR1-3 | MIMAT0004409 | >ptr-miR-203 | MIMAT0008065 |
| >zma-miR399d | MIMAT0001707 | >rrv-miR-rR1-4 | MIMAT0004410 | >ptr-miR-206 | MIMAT0008066 |
| >zma-miR399e | MIMAT0001708 | >rrv-miR-rR1-5 | MIMAT0004412 | >ptr-miR-208a | MIMAT0008067 |
| >zma-miR399f | MIMAT0001709 | >rrv-miR-rR1-6 | MIMAT0004413 | >ptr-miR-208b | MIMAT0008068 |
| >zma-miR156j | MIMAT0001710 | >rrv-miR-rR1-7-5p | MIMAT0004415 | >ptr-miR-20b | MIMAT0008069 |
| >zma-miR159a | MIMAT0001711 | >rrv-miR-rR1-7-3p | MIMAT0004416 | >ptr-miR-210 | MIMAT0008070 |
| >zma-miR159b | MIMAT0001712 | >cgr-miR-21 | MIMAT0004417 | >ptr-miR-211 | MIMAT0008071 |
| >zma-miR159c | MIMAT0001713 | >ame-let-7 | MIMAT0004418 | >ptr-miR-216b | MIMAT0008072 |
| >zma-miR159d | MIMAT0001714 | >ame-miR-10 | MIMAT0004419 | >ptr-miR-217 | MIMAT0008073 |
| >zma-miR319a | MIMAT0001715 | >ame-miR-100 | MIMAT0004420 | >ptr-miR-219-5p | MIMAT0009193 |
| >zma-miR319c | MIMAT0001716 | >ame-miR-137 | MIMAT0004421 | >ptr-miR-219-1-3p | MIMAT0008074 |
| >zma-miR319b | MIMAT0001717 | >ame-miR-13a | MIMAT0004422 | >ptr-miR-219-2-3p | MIMAT0009194 |
| >zma-miR319d | MIMAT0001718 | >ame-miR-14 | MIMAT0004423 | >ptr-miR-220b | MIMAT0008075 |
| >zma-miR166k | MIMAT0001719 | >ame-miR-275 | MIMAT0004424 | >ptr-miR-221 | MIMAT0008076 |
| >zma-miR166j | MIMAT0001720 | >ame-miR-279 | MIMAT0004425 | >ptr-miR-26b | MIMAT0008077 |
| >zma-miR167e | MIMAT0001721 | >ame-miR-283 | MIMAT0004426 | >ptr-miR-27b | MIMAT0008078 |
| >zma-miR167f | MIMAT0001722 | >ame-miR-29b | MIMAT0004427 | >ptr-miR-296 | MIMAT0008079 |
| >zma-miR167g | MIMAT0001723 | >ame-miR-31a | MIMAT0004428 | >ptr-miR-297 | MIMAT0008080 |
| >zma-miR167h | MIMAT0001724 | >ame-miR-33 | MIMAT0004429 | >ptr-miR-298 | MIMAT0008081 |
| >zma-miR167i | MIMAT0001725 | >ame-miR-34 | MIMAT0004430 | >ptr-miR-29c | MIMAT0008082 |
| >zma-miR168a | MIMAT0001726 | >ame-miR-375 | MIMAT0004431 | >ptr-miR-300 | MIMAT0008083 |
| >zma-miR168b | MIMAT0001727 | >ame-miR-71 | MIMAT0004432 | >ptr-miR-301a | MIMAT0008084 |
| >zma-miR169c | MIMAT0001728 | >ame-miR-79 | MIMAT0004433 | >ptr-miR-301b | MIMAT0008085 |
| >zma-miR169f | MIMAT0001729 | >ame-miR-87 | MIMAT0004434 | >ptr-miR-302a | MIMAT0008086 |
| >zma-miR169g | MIMAT0001730 | >ame-miR-92a | MIMAT0004435 | >ptr-miR-302b | MIMAT0008087 |
| >zma-miR169h | MIMAT0001731 | >ame-miR-925 | MIMAT0004436 | >ptr-miR-302c | MIMAT0008088 |
| >zma-miR169i | MIMAT0001732 | >ame-miR-926 | MIMAT0004437 | >ptr-miR-302d | MIMAT0008089 |
| >zma-miR169k | MIMAT0001733 | >ame-miR-927 | MIMAT0004438 | >ptr-miR-302e | MIMAT0008090 |
| >zma-miR169j | MIMAT0001734 | >ame-miR-928 | MIMAT0004439 | >ptr-miR-302f | MIMAT0008091 |
| >zma-miR169d | MIMAT0001735 | >ame-miR-190 | MIMAT0004440 | >ptr-miR-30e | MIMAT0008092 |
| >zma-miR169e | MIMAT0001736 | >ame-miR-929 | MIMAT0004441 | >ptr-miR-320a | MIMAT0008093 |
| >zma-miR171c | MIMAT0001737 | >ame-miR-930 | MIMAT0004442 | >ptr-miR-320b | MIMAT0008094 |
| >zma-miR171j | MIMAT0001738 | >ame-miR-931 | MIMAT0004443 | >ptr-miR-320c | MIMAT0008095 |
| >zma-miR171e | MIMAT0001739 | >ame-miR-932 | MIMAT0004444 | >ptr-miR-320d | MIMAT0008096 |
| >zma-miR171i | MIMAT0001740 | >hsa-miR-933 | MIMAT0004976 | >ptr-miR-323 | MIMAT0008097 |
| >zma-miR171g | MIMAT0001741 | >hsa-miR-934 | MIMAT0004977 | >ptr-miR-324 | MIMAT0008098 |
| >zma-miR172e | MIMAT0001742 | >hsa-miR-935 | MIMAT0004978 | >ptr-miR-326 | MIMAT0008099 |
| >zma-miR166l | MIMAT0001743 | >hsa-miR-936 | MIMAT0004979 | >ptr-miR-328 | MIMAT0008100 |
| >zma-miR166m | MIMAT0001744 | >hsa-miR-937 | MIMAT0004980 | >ptr-miR-329 | MIMAT0008101 |
| >zma-miR171k | MIMAT0001745 | >hsa-miR-938 | MIMAT0004981 | >ptr-miR-330 | MIMAT0008102 |
| >zma-miR171h | MIMAT0001746 | >hsa-miR-939 | MIMAT0004982 | >ptr-miR-331 | MIMAT0008103 |
| >zma-miR393 | MIMAT0001747 | >hsa-miR-940 | MIMAT0004983 | >ptr-miR-335 | MIMAT0008104 |
| >zma-miR408 | MIMAT0001748 | >hsa-miR-941 | MIMAT0004984 | >ptr-miR-337 | MIMAT0008105 |
| >zma-miR156k | MIMAT0001749 | >hsa-miR-942 | MIMAT0004985 | >ptr-miR-338 | MIMAT0008106 |
| >zma-miR160f | MIMAT0001750 | >hsa-miR-943 | MIMAT0004986 | >ptr-miR-339 | MIMAT0008107 |
| >sbi-miR399h | MIMAT0001751 | >hsa-miR-944 | MIMAT0004987 | >ptr-miR-33b | MIMAT0008108 |
| >sbi-miR399i | MIMAT0001752 | >bna-miR156a | MIMAT0004445 | >ptr-miR-340 | MIMAT0008109 |
| >sbi-miR159b | MIMAT0001753 | >bna-miR171g | MIMAT0004446 | >ptr-miR-342 | MIMAT0008110 |
| >sbi-miR164c | MIMAT0001754 | >bna-miR393 | MIMAT0004447 | >ptr-miR-345 | MIMAT0008111 |
| >sbi-miR166g | MIMAT0001755 | >bna-miR396a | MIMAT0004448 | >ptr-miR-346 | MIMAT0008112 |
| >sbi-miR171f | MIMAT0001756 | >bna-miR399 | MIMAT0004449 | >ptr-miR-34b | MIMAT0008113 |
| >sbi-miR395f | MIMAT0001757 | >hsa-miR-297 | MIMAT0004450 | >ptr-miR-34c | MIMAT0008114 |
| >sbi-miR156e | MIMAT0001758 | >pta-miR156a | MIMAT0004988 | >ptr-miR-361 | MIMAT0008115 |
| >dre-let-7a | MIMAT0001759 | >pta-miR156b | MIMAT0004989 | >ptr-miR-362 | MIMAT0008116 |
| >dre-let-7b | MIMAT0001760 | >pta-miR159a | MIMAT0004990 | >ptr-miR-365 | MIMAT0008117 |
| >dre-let-7c | MIMAT0001761 | >pta-miR159b | MIMAT0004991 | >ptr-miR-367 | MIMAT0008118 |
| >dre-let-7d | MIMAT0001762 | >pta-miR159c | MIMAT0004992 | >ptr-miR-369 | MIMAT0008119 |
| >dre-let-7e | MIMAT0001763 | >pta-miR166a | MIMAT0004993 | >ptr-miR-370 | MIMAT0008120 |
| >dre-let-7f | MIMAT0001764 | >pta-miR166b | MIMAT0004994 | >ptr-miR-371 | MIMAT0008121 |
| >dre-let-7g | MIMAT0001765 | >pta-miR166c | MIMAT0004995 | >ptr-miR-372 | MIMAT0008122 |
| >dre-let-7h | MIMAT0001766 | >pta-miR171 | MIMAT0004996 | >ptr-miR-374a | MIMAT0008123 |
| >dre-let-7i | MIMAT0001767 | >pta-miR319 | MIMAT0004997 | >ptr-miR-374b | MIMAT0008124 |
| >dre-miR-1 | MIMAT0001768 | >pta-miR390 | MIMAT0004998 | >ptr-miR-375 | MIMAT0008125 |
| >dre-miR-9 | MIMAT0001769 | >pta-miR396 | MIMAT0004999 | >ptr-miR-376a | MIMAT0008126 |
| >dre-miR-10c | MIMAT0001770 | >pta-miR398 | MIMAT0005000 | >ptr-miR-376b | MIMAT0008127 |
| >dre-miR-10d | MIMAT0001771 | >pta-miR408 | MIMAT0005001 | >ptr-miR-376c | MIMAT0008128 |
| >dre-miR-15a | MIMAT0001772 | >pta-miR482a | MIMAT0005002 | >ptr-miR-377 | MIMAT0008129 |
| >dre-miR-15b | MIMAT0001773 | >pta-miR482b | MIMAT0005003 | >ptr-miR-378 | MIMAT0008130 |
| >dre-miR-16a | MIMAT0001774 | >pta-miR783 | MIMAT0005004 | >ptr-miR-379 | MIMAT0008131 |
| >dre-miR-16b | MIMAT0001775 | >pta-miR946a | MIMAT0005005 | >ptr-miR-380 | MIMAT0008132 |
| >dre-miR-16c | MIMAT0001776 | >pta-miR946b | MIMAT0005006 | >ptr-miR-381 | MIMAT0008133 |
| >dre-miR-17a | MIMAT0001777 | >pta-miR947 | MIMAT0005007 | >ptr-miR-382 | MIMAT0008134 |
| >dre-miR-20b | MIMAT0001778 | >pta-miR948 | MIMAT0005008 | >ptr-miR-383 | MIMAT0008135 |
| >dre-miR-18a | MIMAT0001779 | >pta-miR949 | MIMAT0005009 | >ptr-miR-409 | MIMAT0008136 |
| >dre-miR-18b | MIMAT0001780 | >pta-miR950a | MIMAT0005010 | >ptr-miR-410 | MIMAT0008137 |
| >dre-miR-18c | MIMAT0001781 | >pta-miR950b | MIMAT0005011 | >ptr-miR-411 | MIMAT0008138 |
| >dre-miR-19a | MIMAT0001782 | >pta-miR951 | MIMAT0005012 | >ptr-miR-412 | MIMAT0008139 |
| >dre-miR-19b | MIMAT0001783 | >pta-miR952a | MIMAT0005013 | >ptr-miR-421 | MIMAT0008140 |
| >dre-miR-19c | MIMAT0001784 | >pta-miR952b | MIMAT0005014 | >ptr-miR-422a | MIMAT0008141 |
| >dre-miR-19d | MIMAT0001785 | >osa-miR529b | MIMAT0005015 | >ptr-miR-423 | MIMAT0008142 |
| >dre-miR-20a | MIMAT0001786 | >dme-miR-iab-4as-5p | MIMAT0005463 | >ptr-miR-424 | MIMAT0008143 |
| >dre-miR-21 | MIMAT0001787 | >dme-miR-iab-4as-3p | MIMAT0005464 | >ptr-miR-425 | MIMAT0008144 |
| >dre-miR-22a | MIMAT0001788 | >dme-miR-954 | MIMAT0005465 | >ptr-miR-431 | MIMAT0008145 |
| >dre-miR-22b | MIMAT0001789 | >dme-miR-955 | MIMAT0005466 | >ptr-miR-432 | MIMAT0008146 |
| >dre-miR-23a | MIMAT0001790 | >dme-miR-190 | MIMAT0005467 | >ptr-miR-433 | MIMAT0008147 |
| >dre-miR-23b | MIMAT0001791 | >dme-miR-193 | MIMAT0005468 | >ptr-miR-448 | MIMAT0008148 |
| >dre-miR-24 | MIMAT0001792 | >dme-miR-956 | MIMAT0005469 | >ptr-miR-449a | MIMAT0008149 |
| >dre-miR-25 | MIMAT0001793 | >dme-miR-957 | MIMAT0005470 | >ptr-miR-449b | MIMAT0008150 |
| >dre-miR-26a | MIMAT0001794 | >dme-miR-958 | MIMAT0005471 | >ptr-miR-450a | MIMAT0008151 |
| >dre-miR-26b | MIMAT0001795 | >dme-miR-375 | MIMAT0005472 | >ptr-miR-450b | MIMAT0008152 |
| >dre-miR-27a | MIMAT0001796 | >dme-miR-959 | MIMAT0005473 | >ptr-miR-451 | MIMAT0008153 |
| >dre-miR-27b | MIMAT0001797 | >dme-miR-960 | MIMAT0005474 | >ptr-miR-452 | MIMAT0008154 |
| >dre-miR-27c | MIMAT0001798 | >dme-miR-961 | MIMAT0005475 | >ptr-miR-453 | MIMAT0008155 |
| >dre-miR-27d | MIMAT0001799 | >dme-miR-962 | MIMAT0005476 | >ptr-miR-454 | MIMAT0008156 |
| >dre-miR-27e | MIMAT0001800 | >dme-miR-963 | MIMAT0005477 | >ptr-miR-455 | MIMAT0008157 |
| >dre-miR-29b | MIMAT0001801 | >dme-miR-964 | MIMAT0005478 | >ptr-miR-484 | MIMAT0008158 |
| >dre-miR-29a | MIMAT0001802 | >dme-miR-932 | MIMAT0005479 | >ptr-miR-485 | MIMAT0008159 |
| >dre-miR-30a | MIMAT0001803 | >dme-miR-965 | MIMAT0005480 | >ptr-miR-486 | MIMAT0008160 |
| >dre-miR-30b | MIMAT0001804 | >dme-miR-966 | MIMAT0005481 | >ptr-miR-487a | MIMAT0008161 |
| >dre-miR-30c | MIMAT0001805 | >dme-miR-967 | MIMAT0005482 | >ptr-miR-487b | MIMAT0008162 |
| >dre-miR-30d | MIMAT0001806 | >dme-miR-1002 | MIMAT0005483 | >ptr-miR-488 | MIMAT0008163 |
| >dre-miR-30e | MIMAT0001807 | >dme-miR-968 | MIMAT0005484 | >ptr-miR-489 | MIMAT0008164 |
| >dre-miR-92a | MIMAT0001808 | >dme-miR-969 | MIMAT0005485 | >ptr-miR-490 | MIMAT0008165 |
| >dre-miR-92b | MIMAT0001809 | >dme-miR-970 | MIMAT0005486 | >ptr-miR-491 | MIMAT0008166 |
| >dre-miR-93 | MIMAT0001810 | >dme-miR-971 | MIMAT0005487 | >ptr-miR-492 | MIMAT0008167 |
| >dre-miR-96 | MIMAT0001811 | >dme-miR-972 | MIMAT0005488 | >ptr-miR-494 | MIMAT0008168 |
| >dre-miR-99 | MIMAT0001812 | >dme-miR-973 | MIMAT0005489 | >ptr-miR-495 | MIMAT0008169 |
| >dre-miR-100 | MIMAT0001813 | >dme-miR-974 | MIMAT0005490 | >ptr-miR-496 | MIMAT0008170 |
| >dre-miR-101a | MIMAT0001814 | >dme-miR-975 | MIMAT0005491 | >ptr-miR-498 | MIMAT0008171 |
| >dre-miR-101b | MIMAT0001815 | >dme-miR-976 | MIMAT0005492 | >ptr-miR-499 | MIMAT0008172 |
| >dre-miR-103 | MIMAT0001816 | >dme-miR-977 | MIMAT0005493 | >ptr-miR-500 | MIMAT0008173 |
| >dre-miR-107 | MIMAT0001817 | >dme-miR-978 | MIMAT0005494 | >ptr-miR-501 | MIMAT0008174 |
| >dre-miR-122 | MIMAT0001818 | >dme-miR-979 | MIMAT0005495 | >ptr-miR-502 | MIMAT0008175 |
| >dre-miR-124 | MIMAT0001819 | >dme-miR-980 | MIMAT0005496 | >ptr-miR-503 | MIMAT0008176 |
| >dre-miR-125a | MIMAT0001820 | >dme-miR-981 | MIMAT0005497 | >ptr-miR-504 | MIMAT0008177 |
| >dre-miR-125b | MIMAT0001821 | >dme-miR-982 | MIMAT0005498 | >ptr-miR-505 | MIMAT0008178 |
| >dre-miR-125c | MIMAT0001822 | >dme-miR-983 | MIMAT0005499 | >ptr-miR-511 | MIMAT0008179 |
| >dre-miR-126 | MIMAT0001823 | >dme-miR-984 | MIMAT0005500 | >ptr-miR-512 | MIMAT0008180 |
| >dre-miR-128 | MIMAT0001824 | >dme-miR-927 | MIMAT0005501 | >ptr-miR-515 | MIMAT0008181 |
| >dre-miR-129 | MIMAT0001825 | >dme-miR-985 | MIMAT0005502 | >ptr-miR-516a | MIMAT0008182 |
| >dre-miR-130a | MIMAT0001826 | >dme-miR-986 | MIMAT0005503 | >ptr-miR-516b | MIMAT0008183 |
| >dre-miR-130b | MIMAT0001827 | >dme-miR-987 | MIMAT0005504 | >ptr-miR-517a | MIMAT0008184 |
| >dre-miR-130c | MIMAT0001828 | >dme-miR-988 | MIMAT0005505 | >ptr-miR-517b | MIMAT0008185 |
| >dre-miR-132 | MIMAT0001829 | >dme-miR-989 | MIMAT0005506 | >ptr-miR-518a | MIMAT0008186 |
| >dre-miR-133a | MIMAT0001830 | >dme-miR-137 | MIMAT0005507 | >ptr-miR-518b | MIMAT0008187 |
| >dre-miR-133b | MIMAT0001831 | >dme-miR-990 | MIMAT0005508 | >ptr-miR-518c | MIMAT0008188 |
| >dre-miR-133c | MIMAT0001832 | >dme-miR-991 | MIMAT0005509 | >ptr-miR-518d | MIMAT0008189 |
| >dre-miR-135c | MIMAT0001833 | >dme-miR-992 | MIMAT0005510 | >ptr-miR-518e | MIMAT0008190 |
| >dre-miR-137 | MIMAT0001834 | >dme-miR-929 | MIMAT0005511 | >ptr-miR-518f | MIMAT0008191 |
| >dre-miR-138 | MIMAT0001835 | >dme-miR-993 | MIMAT0005512 | >ptr-miR-519a | MIMAT0008192 |
| >dre-miR-140 | MIMAT0001836 | >dme-miR-994 | MIMAT0005513 | >ptr-miR-519b | MIMAT0008193 |
| >dre-miR-141 | MIMAT0001837 | >dme-miR-995 | MIMAT0005514 | >ptr-miR-519c | MIMAT0008194 |
| >dre-miR-142a-5p | MIMAT0001838 | >dme-miR-996 | MIMAT0005515 | >ptr-miR-519d | MIMAT0008195 |
| >dre-miR-142a-3p | MIMAT0003160 | >dme-miR-252 | MIMAT0005516 | >ptr-miR-519e | MIMAT0008196 |
| >dre-miR-142b-5p | MIMAT0001839 | >dme-miR-997 | MIMAT0005517 | >ptr-miR-520a | MIMAT0008197 |
| >dre-miR-143 | MIMAT0001840 | >dme-miR-998 | MIMAT0005518 | >ptr-miR-520b | MIMAT0008198 |
| >dre-miR-144 | MIMAT0001841 | >dme-miR-999 | MIMAT0005519 | >ptr-miR-520c | MIMAT0008199 |
| >dre-miR-145 | MIMAT0001842 | >dme-miR-1000 | MIMAT0005520 | >ptr-miR-520d | MIMAT0008200 |
| >dre-miR-146a | MIMAT0001843 | >dme-miR-1001 | MIMAT0005521 | >ptr-miR-520e | MIMAT0008201 |
| >dre-miR-146b | MIMAT0001844 | >dme-miR-1003 | MIMAT0005016 | >ptr-miR-520f | MIMAT0008202 |
| >dre-miR-148 | MIMAT0001845 | >dme-miR-1004 | MIMAT0005017 | >ptr-miR-520g | MIMAT0008203 |
| >dre-miR-150 | MIMAT0001846 | >dme-miR-1005 | MIMAT0005018 | >ptr-miR-520h | MIMAT0008204 |
| >dre-miR-152 | MIMAT0001847 | >dme-miR-1006 | MIMAT0005019 | >ptr-miR-521 | MIMAT0008205 |
| >dre-miR-153b | MIMAT0001848 | >dme-miR-1007 | MIMAT0005020 | >ptr-miR-522 | MIMAT0008206 |
| >dre-miR-153a | MIMAT0001849 | >dme-miR-1008 | MIMAT0005021 | >ptr-miR-523 | MIMAT0008207 |
| >dre-miR-153c | MIMAT0001850 | >dme-miR-1009 | MIMAT0005022 | >ptr-miR-524 | MIMAT0008208 |
| >dre-miR-155 | MIMAT0001851 | >dme-miR-1010 | MIMAT0005023 | >ptr-miR-525 | MIMAT0008209 |
| >dre-miR-181c | MIMAT0001852 | >dme-miR-1011 | MIMAT0005024 | >ptr-miR-526a | MIMAT0008210 |
| >dre-miR-184 | MIMAT0001853 | >dme-miR-1012 | MIMAT0005025 | >ptr-miR-526b | MIMAT0008211 |
| >dre-miR-190 | MIMAT0001854 | >dme-miR-1013 | MIMAT0005026 | >ptr-miR-527 | MIMAT0008212 |
| >dre-miR-462 | MIMAT0001855 | >dme-miR-1014 | MIMAT0005027 | >ptr-miR-532 | MIMAT0008213 |
| >dre-miR-193a | MIMAT0001856 | >dme-miR-1015 | MIMAT0005028 | >ptr-miR-539 | MIMAT0008214 |
| >dre-miR-193b | MIMAT0001857 | >dme-miR-1016 | MIMAT0005029 | >ptr-miR-543 | MIMAT0008215 |
| >dre-miR-194a | MIMAT0001858 | >dme-miR-1017 | MIMAT0005030 | >ptr-miR-544 | MIMAT0008216 |
| >dre-miR-194b | MIMAT0001859 | >cel-miR-1018 | MIMAT0005031 | >ptr-miR-545 | MIMAT0008217 |
| >dre-miR-196b | MIMAT0001860 | >cel-miR-1019 | MIMAT0005032 | >ptr-miR-548a | MIMAT0008218 |
| >dre-miR-200a | MIMAT0001861 | >cel-miR-1020 | MIMAT0005033 | >ptr-miR-548b | MIMAT0008219 |
| >dre-miR-200b | MIMAT0001862 | >mdv2-miR-M14-5p | MIMAT0004451 | >ptr-miR-548c | MIMAT0008220 |
| >dre-miR-200c | MIMAT0001863 | >mdv2-miR-M14-3p | MIMAT0004452 | >ptr-miR-548f | MIMAT0008221 |
| >dre-miR-202 | MIMAT0001864 | >mdv2-miR-M15 | MIMAT0004454 | >ptr-miR-548h | MIMAT0008222 |
| >dre-miR-203b | MIMAT0001865 | >mdv2-miR-M16 | MIMAT0004455 | >ptr-miR-548i | MIMAT0008223 |
| >dre-miR-206 | MIMAT0001866 | >mdv2-miR-M17 | MIMAT0004457 | >ptr-miR-548j | MIMAT0008224 |
| >dre-miR-216b | MIMAT0001867 | >mdv2-miR-M18-5p | MIMAT0004458 | >ptr-miR-548k | MIMAT0008225 |
| >dre-miR-218a | MIMAT0001868 | >mdv2-miR-M18-3p | MIMAT0004459 | >ptr-miR-548l | MIMAT0008226 |
| >dre-miR-218b | MIMAT0001869 | >mdv2-miR-M19 | MIMAT0004460 | >ptr-miR-548n | MIMAT0008227 |
| >dre-miR-301a | MIMAT0001870 | >mdv2-miR-M20 | MIMAT0004462 | >ptr-miR-548p | MIMAT0008228 |
| >dre-miR-301b | MIMAT0001871 | >mdv2-miR-M21 | MIMAT0004464 | >ptr-miR-549 | MIMAT0008229 |
| >dre-miR-301c | MIMAT0001872 | >mdv2-miR-M22 | MIMAT0004465 | >ptr-miR-550 | MIMAT0008230 |
| >dre-miR-338 | MIMAT0001873 | >mdv2-miR-M23 | MIMAT0004467 | >ptr-miR-551a | MIMAT0008231 |
| >dre-miR-363 | MIMAT0001874 | >mdv2-miR-M24 | MIMAT0004468 | >ptr-miR-551b | MIMAT0008232 |
| >dre-miR-365 | MIMAT0001875 | >mdv2-miR-M25-5p | MIMAT0004469 | >ptr-miR-552 | MIMAT0008233 |
| >dre-miR-375 | MIMAT0001876 | >mdv2-miR-M25-3p | MIMAT0004470 | >ptr-miR-553 | MIMAT0008234 |
| >dre-miR-454a | MIMAT0001877 | >mdv2-miR-M26 | MIMAT0004471 | >ptr-miR-554 | MIMAT0008235 |
| >dre-miR-454b | MIMAT0001878 | >mdv2-miR-M27-5p | MIMAT0004472 | >ptr-miR-555 | MIMAT0008236 |
| >dre-miR-455 | MIMAT0001879 | >mdv2-miR-M27-3p | MIMAT0004473 | >ptr-miR-556 | MIMAT0008237 |
| >dre-miR-430i | MIMAT0001880 | >mdv2-miR-M28 | MIMAT0004474 | >ptr-miR-557 | MIMAT0008238 |
| >dre-miR-430j | MIMAT0001881 | >mdv2-miR-M29 | MIMAT0004476 | >ptr-miR-558 | MIMAT0008239 |
| >dre-miR-456 | MIMAT0001882 | >mdv2-miR-M30 | MIMAT0004477 | >ptr-miR-559 | MIMAT0008240 |
| >dre-miR-457a | MIMAT0001883 | >cel-miR-1021 | MIMAT0005522 | >ptr-miR-561 | MIMAT0008241 |
| >dre-miR-457b | MIMAT0001884 | >cel-miR-1022 | MIMAT0005523 | >ptr-miR-562 | MIMAT0008242 |
| >dre-miR-458 | MIMAT0001885 | >ppt-miR160e | MIMAT0005034 | >ptr-miR-564 | MIMAT0008243 |
| >dre-miR-459 | MIMAT0001886 | >ppt-miR160f | MIMAT0005035 | >ptr-miR-566 | MIMAT0008244 |
| >dre-miR-460-5p | MIMAT0001887 | >ppt-miR160g | MIMAT0005036 | >ptr-miR-567 | MIMAT0008245 |
| >dre-miR-460-3p | MIMAT0001888 | >ppt-miR160h | MIMAT0005037 | >ptr-miR-568 | MIMAT0008246 |
| >dre-miR-461 | MIMAT0001889 | >ppt-miR160i | MIMAT0005038 | >ptr-miR-569 | MIMAT0008247 |
| >ptc-miR156a | MIMAT0001890 | >ppt-miR166c | MIMAT0005039 | >ptr-miR-572 | MIMAT0008248 |
| >ptc-miR156b | MIMAT0001891 | >ppt-miR166d | MIMAT0005040 | >ptr-miR-575 | MIMAT0008249 |
| >ptc-miR156c | MIMAT0001892 | >ppt-miR166e | MIMAT0005041 | >ptr-miR-576 | MIMAT0008250 |
| >ptc-miR156d | MIMAT0001893 | >ppt-miR166f | MIMAT0005042 | >ptr-miR-577 | MIMAT0008251 |
| >ptc-miR156e | MIMAT0001894 | >ppt-miR166g | MIMAT0005043 | >ptr-miR-579 | MIMAT0008252 |
| >ptc-miR156f | MIMAT0001895 | >ppt-miR166h | MIMAT0005044 | >ptr-miR-580 | MIMAT0008253 |
| >ptc-miR156g | MIMAT0001896 | >ppt-miR166i | MIMAT0005045 | >ptr-miR-581 | MIMAT0008254 |
| >ptc-miR156h | MIMAT0001897 | >ppt-miR166j | MIMAT0005046 | >ptr-miR-582 | MIMAT0008255 |
| >ptc-miR156i | MIMAT0001898 | >ppt-miR166k | MIMAT0005047 | >ptr-miR-583 | MIMAT0008256 |
| >ptc-miR156j | MIMAT0001899 | >ppt-miR166l | MIMAT0005048 | >ptr-miR-584 | MIMAT0008257 |
| >ptc-miR156k | MIMAT0001900 | >ppt-miR166m | MIMAT0005049 | >ptr-miR-586 | MIMAT0008258 |
| >ptc-miR159a | MIMAT0001901 | >ppt-miR408b | MIMAT0005050 | >ptr-miR-587 | MIMAT0008259 |
| >ptc-miR159b | MIMAT0001902 | >ppt-miR477d | MIMAT0005051 | >ptr-miR-588 | MIMAT0008260 |
| >ptc-miR159c | MIMAT0001903 | >ppt-miR477e | MIMAT0005052 | >ptr-miR-590 | MIMAT0008261 |
| >ptc-miR159d | MIMAT0001904 | >ppt-miR477h | MIMAT0005053 | >ptr-miR-591 | MIMAT0008262 |
| >ptc-miR159e | MIMAT0001905 | >ppt-miR529a | MIMAT0005054 | >ptr-miR-592 | MIMAT0008263 |
| >ptc-miR159f | MIMAT0001906 | >ppt-miR529b | MIMAT0005055 | >ptr-miR-593 | MIMAT0008264 |
| >ptc-miR160a | MIMAT0001907 | >ppt-miR529c | MIMAT0005056 | >ptr-miR-595 | MIMAT0008265 |
| >ptc-miR160b | MIMAT0001908 | >ppt-miR529d | MIMAT0005057 | >ptr-miR-597 | MIMAT0008266 |
| >ptc-miR160c | MIMAT0001909 | >ppt-miR529e | MIMAT0005058 | >ptr-miR-598 | MIMAT0008267 |
| >ptc-miR160d | MIMAT0001910 | >ppt-miR529f | MIMAT0005059 | >ptr-miR-599 | MIMAT0008268 |
| >ptc-miR160e | MIMAT0001911 | >ppt-miR529g | MIMAT0005060 | >ptr-miR-600 | MIMAT0008269 |
| >ptc-miR160f | MIMAT0001912 | >ppt-miR533c | MIMAT0005061 | >ptr-miR-601 | MIMAT0008270 |
| >ptc-miR160g | MIMAT0001913 | >ppt-miR533d | MIMAT0005062 | >ptr-miR-605 | MIMAT0008271 |
| >ptc-miR160h | MIMAT0001914 | >ppt-miR533e | MIMAT0005063 | >ptr-miR-609 | MIMAT0008272 |
| >ptc-miR162a | MIMAT0001915 | >ppt-miR536d | MIMAT0005064 | >ptr-miR-610 | MIMAT0008273 |
| >ptc-miR162b | MIMAT0001916 | >ppt-miR536e | MIMAT0005065 | >ptr-miR-612 | MIMAT0008274 |
| >ptc-miR162c | MIMAT0001917 | >ppt-miR536f | MIMAT0005066 | >ptr-miR-613 | MIMAT0008275 |
| >ptc-miR164a | MIMAT0001918 | >ppt-miR537c | MIMAT0005067 | >ptr-miR-614 | MIMAT0008276 |
| >ptc-miR164b | MIMAT0001919 | >ppt-miR537d | MIMAT0005068 | >ptr-miR-615 | MIMAT0008277 |
| >ptc-miR164c | MIMAT0001920 | >ppt-miR902c-5p | MIMAT0005069 | >ptr-miR-616 | MIMAT0008278 |
| >ptc-miR164d | MIMAT0001921 | >ppt-miR902c-3p | MIMAT0005070 | >ptr-miR-617 | MIMAT0008279 |
| >ptc-miR164e | MIMAT0001922 | >ppt-miR902d-5p | MIMAT0005071 | >ptr-miR-618 | MIMAT0008280 |
| >ptc-miR164f | MIMAT0001923 | >ppt-miR902d-3p | MIMAT0005072 | >ptr-miR-619 | MIMAT0008281 |
| >ptc-miR166a | MIMAT0001924 | >ppt-miR902e-5p | MIMAT0005073 | >ptr-miR-621 | MIMAT0008282 |
| >ptc-miR166b | MIMAT0001925 | >ppt-miR902e-3p | MIMAT0005074 | >ptr-miR-622 | MIMAT0008283 |
| >ptc-miR166c | MIMAT0001926 | >ppt-miR902f-5p | MIMAT0005075 | >ptr-miR-624 | MIMAT0008284 |
| >ptc-miR166d | MIMAT0001927 | >ppt-miR902f-3p | MIMAT0005076 | >ptr-miR-626 | MIMAT0008285 |
| >ptc-miR166e | MIMAT0001928 | >ppt-miR902g-5p | MIMAT0005077 | >ptr-miR-627 | MIMAT0008286 |
| >ptc-miR166f | MIMAT0001929 | >ppt-miR902g-3p | MIMAT0005078 | >ptr-miR-628 | MIMAT0008287 |
| >ptc-miR166g | MIMAT0001930 | >ppt-miR902h-5p | MIMAT0005079 | >ptr-miR-630 | MIMAT0008288 |
| >ptc-miR166h | MIMAT0001931 | >ppt-miR902h-3p | MIMAT0005080 | >ptr-miR-632 | MIMAT0008289 |
| >ptc-miR166i | MIMAT0001932 | >ppt-miR902i-5p | MIMAT0005081 | >ptr-miR-633 | MIMAT0008290 |
| >ptc-miR166j | MIMAT0001933 | >ppt-miR902i-3p | MIMAT0005082 | >ptr-miR-634 | MIMAT0008291 |
| >ptc-miR166k | MIMAT0001934 | >ppt-miR902j-5p | MIMAT0005083 | >ptr-miR-635 | MIMAT0008292 |
| >ptc-miR166l | MIMAT0001935 | >ppt-miR902j-3p | MIMAT0005084 | >ptr-miR-637 | MIMAT0008293 |
| >ptc-miR166m | MIMAT0001936 | >ppt-miR902k-5p | MIMAT0005085 | >ptr-miR-640 | MIMAT0008294 |
| >ptc-miR166n | MIMAT0001937 | >ppt-miR902k-3p | MIMAT0005086 | >ptr-miR-641 | MIMAT0008295 |
| >ptc-miR166o | MIMAT0001938 | >ppt-miR902l-5p | MIMAT0005087 | >ptr-miR-642 | MIMAT0008296 |
| >ptc-miR166p | MIMAT0001939 | >ppt-miR902l-3p | MIMAT0005088 | >ptr-miR-643 | MIMAT0008297 |
| >ptc-miR166q | MIMAT0001940 | >ppt-miR1222b | MIMAT0005089 | >ptr-miR-645 | MIMAT0008298 |
| >ptc-miR167a | MIMAT0001941 | >ppt-miR1222c | MIMAT0005090 | >ptr-miR-646 | MIMAT0008299 |
| >ptc-miR167b | MIMAT0001942 | >ppt-miR1222d | MIMAT0005091 | >ptr-miR-649 | MIMAT0008300 |
| >ptc-miR167c | MIMAT0001943 | >ppt-miR1222e | MIMAT0005092 | >ptr-miR-650 | MIMAT0008301 |
| >ptc-miR167d | MIMAT0001944 | >ppt-miR1223b | MIMAT0005093 | >ptr-miR-652 | MIMAT0008302 |
| >ptc-miR167e | MIMAT0001945 | >ppt-miR1223c | MIMAT0005094 | >ptr-miR-653 | MIMAT0008303 |
| >ptc-miR167f | MIMAT0001946 | >ppt-miR1223d | MIMAT0005095 | >ptr-miR-654 | MIMAT0008304 |
| >ptc-miR167g | MIMAT0001947 | >ppt-miR1223e | MIMAT0005096 | >ptr-miR-656 | MIMAT0008305 |
| >ptc-miR167h | MIMAT0001948 | >ppt-miR1223f | MIMAT0005097 | >ptr-miR-657 | MIMAT0008306 |
| >ptc-miR168a | MIMAT0001949 | >ppt-miR1223g | MIMAT0005098 | >ptr-miR-658 | MIMAT0008307 |
| >ptc-miR168b | MIMAT0001950 | >ppt-miR1223h | MIMAT0005099 | >ptr-miR-660 | MIMAT0008308 |
| >ptc-miR169a | MIMAT0001951 | >ppt-miR1223i | MIMAT0005100 | >ptr-miR-663a | MIMAT0008309 |
| >ptc-miR169aa | MIMAT0001952 | >ppt-miR1223j | MIMAT0005101 | >ptr-miR-663b | MIMAT0008310 |
| >ptc-miR169ab | MIMAT0001953 | >ppt-miR1023a-5p | MIMAT0005102 | >ptr-miR-664 | MIMAT0008311 |
| >ptc-miR169ac | MIMAT0001954 | >ppt-miR1023a-3p | MIMAT0005103 | >ptr-miR-665 | MIMAT0008312 |
| >ptc-miR169ad | MIMAT0001955 | >ppt-miR1023b-5p | MIMAT0005104 | >ptr-miR-668 | MIMAT0008313 |
| >ptc-miR169ae | MIMAT0001956 | >ppt-miR1023b-3p | MIMAT0005105 | >ptr-miR-671 | MIMAT0008314 |
| >ptc-miR169af | MIMAT0001957 | >ppt-miR1023c-5p | MIMAT0005106 | >ptr-miR-708 | MIMAT0008315 |
| >ptc-miR169b | MIMAT0001958 | >ppt-miR1023c-3p | MIMAT0005107 | >ptr-miR-720 | MIMAT0008316 |
| >ptc-miR169c | MIMAT0001959 | >ppt-miR1023d-5p | MIMAT0005108 | >ptr-miR-744 | MIMAT0008317 |
| >ptc-miR169d | MIMAT0001960 | >ppt-miR1024a | MIMAT0005109 | >ptr-miR-758 | MIMAT0008318 |
| >ptc-miR169e | MIMAT0001961 | >ppt-miR1024b | MIMAT0005110 | >ptr-miR-760 | MIMAT0008319 |
| >ptc-miR169f | MIMAT0001962 | >ppt-miR1025 | MIMAT0005111 | >ptr-miR-765 | MIMAT0008320 |
| >ptc-miR169g | MIMAT0001963 | >ppt-miR1026a | MIMAT0005112 | >ptr-miR-766 | MIMAT0008321 |
| >ptc-miR169h | MIMAT0001964 | >ppt-miR1026b | MIMAT0005113 | >ptr-miR-770 | MIMAT0008322 |
| >ptc-miR169i | MIMAT0001965 | >ppt-miR1027a | MIMAT0005114 | >ptr-miR-802 | MIMAT0008323 |
| >ptc-miR169j | MIMAT0001966 | >ppt-miR1027b | MIMAT0005115 | >ptr-miR-873 | MIMAT0008324 |
| >ptc-miR169k | MIMAT0001967 | >ppt-miR1028a-5p | MIMAT0005116 | >ptr-miR-874 | MIMAT0008325 |
| >ptc-miR169l | MIMAT0001968 | >ppt-miR1028a-3p | MIMAT0005117 | >ptr-miR-875 | MIMAT0008326 |
| >ptc-miR169m | MIMAT0001969 | >ppt-miR1028b-5p | MIMAT0005118 | >ptr-miR-876 | MIMAT0008327 |
| >ptc-miR169n | MIMAT0001970 | >ppt-miR1028b-3p | MIMAT0005119 | >ptr-miR-885 | MIMAT0008328 |
| >ptc-miR169o | MIMAT0001971 | >ppt-miR1028c-5p | MIMAT0005120 | >ptr-miR-886 | MIMAT0008329 |
| >ptc-miR169p | MIMAT0001972 | >ppt-miR1028c-3p | MIMAT0005121 | >ptr-miR-887 | MIMAT0008330 |
| >ptc-miR169q | MIMAT0001973 | >ppt-miR1029 | MIMAT0005122 | >ptr-miR-889 | MIMAT0008331 |
| >ptc-miR169r | MIMAT0001974 | >ppt-miR1030a | MIMAT0005123 | >ptr-miR-890 | MIMAT0008332 |
| >ptc-miR169s | MIMAT0001975 | >ppt-miR1030b | MIMAT0005124 | >ptr-miR-891a | MIMAT0008333 |
| >ptc-miR169t | MIMAT0001976 | >ppt-miR1030c | MIMAT0005125 | >ptr-miR-891b | MIMAT0008334 |
| >ptc-miR169u | MIMAT0001977 | >ppt-miR1030d | MIMAT0005126 | >ptr-miR-892a | MIMAT0008335 |
| >ptc-miR169v | MIMAT0001978 | >ppt-miR1030e | MIMAT0005127 | >ptr-miR-920 | MIMAT0008336 |
| >ptc-miR169w | MIMAT0001979 | >ppt-miR1030f | MIMAT0005128 | >ptr-miR-922 | MIMAT0008337 |
| >ptc-miR169x | MIMAT0001980 | >ppt-miR1030g | MIMAT0005129 | >ptr-miR-923 | MIMAT0008338 |
| >ptc-miR169y | MIMAT0001981 | >ppt-miR1030h | MIMAT0005130 | >ptr-miR-924 | MIMAT0008339 |
| >ptc-miR169z | MIMAT0001982 | >ppt-miR1030i | MIMAT0005131 | >ptr-miR-933 | MIMAT0008340 |
| >ptc-miR171a | MIMAT0001983 | >ppt-miR1030j | MIMAT0005132 | >ptr-miR-934 | MIMAT0008341 |
| >ptc-miR171b | MIMAT0001984 | >ppt-miR1031a | MIMAT0005133 | >ptr-miR-935 | MIMAT0008342 |
| >ptc-miR171c | MIMAT0001985 | >ppt-miR1031b | MIMAT0005134 | >ptr-miR-936 | MIMAT0008343 |
| >ptc-miR171d | MIMAT0001986 | >ppt-miR1032 | MIMAT0005135 | >ptr-miR-937 | MIMAT0008344 |
| >ptc-miR171e | MIMAT0001987 | >ppt-miR1033a | MIMAT0005136 | >ptr-miR-938 | MIMAT0008345 |
| >ptc-miR171f | MIMAT0001988 | >ppt-miR1033b | MIMAT0005137 | >ptr-miR-939 | MIMAT0008346 |
| >ptc-miR171g | MIMAT0001989 | >ppt-miR1033c | MIMAT0005138 | >ptr-miR-940 | MIMAT0008347 |
| >ptc-miR171h | MIMAT0001990 | >ppt-miR1033d | MIMAT0005139 | >ptr-miR-942 | MIMAT0008348 |
| >ptc-miR171i | MIMAT0001991 | >ppt-miR1033e | MIMAT0005140 | >ptr-miR-943 | MIMAT0008349 |
| >ptc-miR171j | MIMAT0001992 | >ppt-miR1034 | MIMAT0005141 | >ptr-miR-944 | MIMAT0008350 |
| >ptc-miR172a | MIMAT0001993 | >ppt-miR1035 | MIMAT0005142 | >ptr-miR-99b | MIMAT0008351 |
| >ptc-miR172b | MIMAT0001994 | >ppt-miR1036-5p | MIMAT0005143 | >tca-miR-1 | MIMAT0008352 |
| >ptc-miR172c | MIMAT0001995 | >ppt-miR1036-3p | MIMAT0005144 | >tca-miR-2 | MIMAT0008353 |
| >ptc-miR172d | MIMAT0001996 | >ppt-miR1037 | MIMAT0005145 | >tca-let-7 | MIMAT0008354 |
| >ptc-miR172e | MIMAT0001997 | >ppt-miR1038-5p | MIMAT0005146 | >tca-miR-7 | MIMAT0008355 |
| >ptc-miR172f | MIMAT0001998 | >ppt-miR1038-3p | MIMAT0005147 | >tca-miR-8 | MIMAT0008356 |
| >ptc-miR172g | MIMAT0001999 | >ppt-miR1039-5p | MIMAT0005148 | >tca-miR-10 | MIMAT0008357 |
| >ptc-miR172h | MIMAT0002000 | >ppt-miR1039-3p | MIMAT0005149 | >tca-miR-12 | MIMAT0008358 |
| >ptc-miR172i | MIMAT0002001 | >ppt-miR1040 | MIMAT0005150 | >tca-miR-13a | MIMAT0008359 |
| >ptc-miR319a | MIMAT0002002 | >ppt-miR1041 | MIMAT0005151 | >tca-miR-13b | MIMAT0008360 |
| >ptc-miR319b | MIMAT0002003 | >ppt-miR1042-5p | MIMAT0005152 | >tca-miR-14 | MIMAT0008361 |
| >ptc-miR319c | MIMAT0002004 | >ppt-miR1042-3p | MIMAT0005153 | >tca-miR-31a | MIMAT0008362 |
| >ptc-miR319d | MIMAT0002005 | >ppt-miR1043-5p | MIMAT0005154 | >tca-miR-33 | MIMAT0008363 |
| >ptc-miR319e | MIMAT0002006 | >ppt-miR1043-3p | MIMAT0005155 | >tca-miR-34 | MIMAT0008364 |
| >ptc-miR319f | MIMAT0002007 | >ppt-miR1044-5p | MIMAT0005156 | >tca-miR-71 | MIMAT0008365 |
| >ptc-miR319g | MIMAT0002008 | >ppt-miR1044-3p | MIMAT0005157 | >tca-miR-87 | MIMAT0008366 |
| >ptc-miR319h | MIMAT0002009 | >ppt-miR1045 | MIMAT0005158 | >tca-miR-92a | MIMAT0008367 |
| >ptc-miR319i | MIMAT0002010 | >ppt-miR1046-5p | MIMAT0005159 | >tca-miR-92b | MIMAT0008368 |
| >ptc-miR390a | MIMAT0002011 | >ppt-miR1046-3p | MIMAT0005160 | >tca-miR-100 | MIMAT0008369 |
| >ptc-miR390b | MIMAT0002012 | >ppt-miR1047-5p | MIMAT0005161 | >tca-miR-124 | MIMAT0008370 |
| >ptc-miR390c | MIMAT0002013 | >ppt-miR1047-3p | MIMAT0005162 | >tca-miR-125 | MIMAT0008371 |
| >ptc-miR390d | MIMAT0002014 | >ppt-miR1048-5p | MIMAT0005163 | >tca-miR-133 | MIMAT0008372 |
| >ptc-miR393a | MIMAT0002015 | >ppt-miR1048-3p | MIMAT0005164 | >tca-miR-137 | MIMAT0008373 |
| >ptc-miR393b | MIMAT0002016 | >ppt-miR1049 | MIMAT0005165 | >tca-miR-184 | MIMAT0008374 |
| >ptc-miR393c | MIMAT0002017 | >ppt-miR1050 | MIMAT0005166 | >tca-miR-190 | MIMAT0008375 |
| >ptc-miR393d | MIMAT0002018 | >ppt-miR1051-5p | MIMAT0005167 | >tca-miR-210 | MIMAT0008376 |
| >ptc-miR394a-5p | MIMAT0002019 | >ppt-miR1051-3p | MIMAT0005168 | >tca-miR-219 | MIMAT0008377 |
| >ptc-miR394a-3p | MIMAT0006783 | >ppt-miR1052 | MIMAT0005169 | >tca-miR-263b | MIMAT0008378 |
| >ptc-miR394b-5p | MIMAT0002020 | >ppt-miR1053-5p | MIMAT0005170 | >tca-miR-275 | MIMAT0008379 |
| >ptc-miR394b-3p | MIMAT0006784 | >ppt-miR1053-3p | MIMAT0005171 | >tca-miR-276 | MIMAT0008381 |
| >ptc-miR395a | MIMAT0002021 | >ppt-miR1054 | MIMAT0005172 | >tca-miR-277 | MIMAT0008382 |
| >ptc-miR395b | MIMAT0002022 | >ppt-miR1055 | MIMAT0005173 | >tca-miR-279 | MIMAT0008383 |
| >ptc-miR395c | MIMAT0002023 | >ppt-miR1056 | MIMAT0005174 | >tca-miR-281 | MIMAT0008385 |
| >ptc-miR395d | MIMAT0002024 | >ppt-miR1057 | MIMAT0005175 | >tca-miR-282 | MIMAT0008386 |
| >ptc-miR395e | MIMAT0002025 | >ppt-miR1058 | MIMAT0005176 | >tca-miR-283 | MIMAT0008387 |
| >ptc-miR395f | MIMAT0002026 | >ppt-miR1059 | MIMAT0005177 | >tca-miR-305 | MIMAT0008388 |
| >ptc-miR395g | MIMAT0002027 | >ppt-miR1060 | MIMAT0005178 | >tca-miR-307 | MIMAT0008389 |
| >ptc-miR395h | MIMAT0002028 | >ppt-miR1061-5p | MIMAT0005179 | >tca-miR-317 | MIMAT0008390 |
| >ptc-miR395i | MIMAT0002029 | >ppt-miR1061-3p | MIMAT0005180 | >tca-miR-927 | MIMAT0008391 |
| >ptc-miR395j | MIMAT0002030 | >ppt-miR1062 | MIMAT0005181 | >tca-miR-932 | MIMAT0008392 |
| >ptc-miR396a | MIMAT0002031 | >ppt-miR1063a | MIMAT0005182 | >tca-miR-iab-4-5p | MIMAT0008393 |
| >ptc-miR396b | MIMAT0002032 | >ppt-miR1063b | MIMAT0005183 | >tca-miR-iab-4-3p | MIMAT0008394 |
| >ptc-miR396c | MIMAT0002033 | >ppt-miR1063c | MIMAT0005184 | >tca-miR-929 | MIMAT0008395 |
| >ptc-miR396d | MIMAT0002034 | >ppt-miR1063d | MIMAT0005185 | >tca-miR-9b | MIMAT0008396 |
| >ptc-miR396e | MIMAT0002035 | >ppt-miR1063e | MIMAT0005186 | >tca-miR-9a | MIMAT0008397 |
| >ptc-miR396f | MIMAT0002036 | >ppt-miR1063f | MIMAT0005187 | >hsv1-miR-112-5p | MIMAT0008398 |
| >ptc-miR396g | MIMAT0002037 | >ppt-miR1063g | MIMAT0005188 | >hsv1-miR-112-3p | MIMAT0008399 |
| >ptc-miR397a | MIMAT0002038 | >ppt-miR1063h | MIMAT0005189 | >hsv1-miR-113 | MIMAT0008400 |
| >ptc-miR397b | MIMAT0002039 | >ppt-miR1064-5p | MIMAT0005190 | >hsv1-miR-114-5p | MIMAT0008401 |
| >ptc-miR397c | MIMAT0002040 | >ppt-miR1064-3p | MIMAT0005191 | >hsv1-miR-114-3p | MIMAT0008402 |
| >ptc-miR398a | MIMAT0002041 | >ppt-miR1065 | MIMAT0005192 | >hsv1-miR-115 | MIMAT0008403 |
| >ptc-miR398b | MIMAT0002042 | >ppt-miR1066 | MIMAT0005193 | >hsv1-miR-116 | MIMAT0008404 |
| >ptc-miR398c | MIMAT0002043 | >ppt-miR1067 | MIMAT0005194 | >dan-miR-92b | MIMAT0008405 |
| >ptc-miR399a | MIMAT0002044 | >ppt-miR1068 | MIMAT0005195 | >dan-miR-288 | MIMAT0008406 |
| >ptc-miR399b | MIMAT0002045 | >ppt-miR1069-5p | MIMAT0005196 | >dan-miR-277 | MIMAT0008407 |
| >ptc-miR399c | MIMAT0002046 | >ppt-miR1069-3p | MIMAT0005197 | >dan-miR-8 | MIMAT0008408 |
| >ptc-miR399d | MIMAT0002047 | >ppt-miR1070 | MIMAT0005198 | >dan-bantam | MIMAT0008409 |
| >ptc-miR399e | MIMAT0002048 | >ppt-miR1071-5p | MIMAT0005199 | >dan-miR-7 | MIMAT0008410 |
| >ptc-miR399f | MIMAT0002049 | >ppt-miR1071-3p | MIMAT0005200 | >dan-miR-87 | MIMAT0008411 |
| >ptc-miR399g | MIMAT0002050 | >ppt-miR1072 | MIMAT0005201 | >dan-miR-92a | MIMAT0008412 |
| >ptc-miR399h | MIMAT0002051 | >ppt-miR1073-5p | MIMAT0005202 | >dan-miR-304 | MIMAT0008413 |
| >ptc-miR399i | MIMAT0002052 | >ppt-miR1073-3p | MIMAT0005203 | >dan-miR-2a | MIMAT0008414 |
| >ptc-miR399j | MIMAT0002053 | >ppt-miR1074 | MIMAT0005204 | >dan-miR-284 | MIMAT0008415 |
| >ptc-miR399k | MIMAT0002054 | >ppt-miR1075 | MIMAT0005205 | >dan-miR-9b | MIMAT0008416 |
| >ptc-miR399l | MIMAT0002055 | >ppt-miR1076-5p | MIMAT0005206 | >dan-miR-2b | MIMAT0008417 |
| >ptc-miR403a | MIMAT0002056 | >ppt-miR1076-3p | MIMAT0005207 | >dan-miR-12 | MIMAT0008418 |
| >ptc-miR403b | MIMAT0002057 | >ppt-miR1077-5p | MIMAT0005208 | >dan-miR-285 | MIMAT0008419 |
| >ptc-miR408 | MIMAT0002058 | >ppt-miR1077-3p | MIMAT0005209 | >dan-miR-13a | MIMAT0008420 |
| >ptc-miR472a | MIMAT0002060 | >ppt-miR1078 | MIMAT0005210 | >dan-miR-308 | MIMAT0008421 |
| >ptc-miR472b | MIMAT0002061 | >ppt-miR1079-5p | MIMAT0005211 | >dan-miR-312 | MIMAT0008422 |
| >ptc-miR473a | MIMAT0002062 | >ppt-miR1079-3p | MIMAT0005212 | >dan-miR-3 | MIMAT0008423 |
| >ptc-miR473b | MIMAT0002063 | >smo-miR156a | MIMAT0005213 | >dan-miR-287 | MIMAT0008424 |
| >ptc-miR474a | MIMAT0002064 | >smo-miR156b | MIMAT0005214 | >dan-miR-310 | MIMAT0008425 |
| >ptc-miR474b | MIMAT0002065 | >smo-miR1568 | MIMAT0005215 | >dan-miR-13b | MIMAT0008426 |
| >ptc-miR474c | MIMAT0002066 | >smo-miR156d | MIMAT0005216 | >dan-miR-4 | MIMAT0008427 |
| >ptc-miR475a | MIMAT0002067 | >smo-miR159 | MIMAT0005217 | >dan-miR-281 | MIMAT0008429 |
| >ptc-miR475b | MIMAT0002068 | >smo-miR160a | MIMAT0005218 | >dan-miR-79 | MIMAT0008430 |
| >ptc-miR475c | MIMAT0002069 | >smo-miR160b | MIMAT0005219 | >dan-miR-124 | MIMAT0008431 |
| >ptc-miR475d | MIMAT0002070 | >smo-miR166a | MIMAT0005220 | >dan-miR-1 | MIMAT0008432 |
| >ptc-miR476a | MIMAT0002071 | >smo-miR166b | MIMAT0005221 | >dan-miR-210 | MIMAT0008433 |
| >ptc-miR476b | MIMAT0002072 | >smo-miR166c | MIMAT0005222 | >dan-miR-307 | MIMAT0008434 |
| >ptc-miR476c | MIMAT0002073 | >smo-miR171a | MIMAT0005223 | >dan-miR-184 | MIMAT0008436 |
| >ptc-miR477a | MIMAT0002074 | >smo-miR171b | MIMAT0005224 | >dan-miR-283 | MIMAT0008437 |
| >ptc-miR477b | MIMAT0002075 | >smo-miR171c | MIMAT0005225 | >dan-miR-280 | MIMAT0008438 |
| >ptc-miR478a | MIMAT0002076 | >smo-miR171d | MIMAT0005226 | >dan-miR-34 | MIMAT0008439 |
| >ptc-miR478b | MIMAT0002077 | >smo-miR396 | MIMAT0005227 | >dan-miR-iab-4-5p | MIMAT0008440 |
| >ptc-miR478c | MIMAT0002078 | >smo-miR408 | MIMAT0005228 | >dan-miR-iab-4-3p | MIMAT0008441 |
| >ptc-miR478d | MIMAT0002079 | >smo-miR536 | MIMAT0005229 | >dan-miR-2c | MIMAT0008442 |
| >ptc-miR478e | MIMAT0002080 | >smo-miR1080 | MIMAT0005230 | >dan-miR-263a | MIMAT0008443 |
| >ptc-miR478f | MIMAT0002081 | >smo-miR1081 | MIMAT0005231 | >dan-miR-286 | MIMAT0008444 |
| >ptc-miR478h | MIMAT0002082 | >smo-miR1082a | MIMAT0005232 | >dan-miR-282 | MIMAT0008445 |
| >ptc-miR478i | MIMAT0002083 | >smo-miR1082b | MIMAT0005233 | >dan-miR-276b | MIMAT0008446 |
| >ptc-miR478j | MIMAT0002084 | >smo-miR1083 | MIMAT0005234 | >dan-miR-263b | MIMAT0008447 |
| >ptc-miR478k | MIMAT0002085 | >smo-miR1084 | MIMAT0005235 | >dan-miR-274 | MIMAT0008448 |
| >ptc-miR478l | MIMAT0002086 | >smo-miR1085-5p | MIMAT0005236 | >dan-miR-316 | MIMAT0008449 |
| >ptc-miR478m | MIMAT0002087 | >smo-miR1085-3p | MIMAT0005237 | >dan-miR-306 | MIMAT0008450 |
| >ptc-miR478n | MIMAT0002088 | >smo-miR1086 | MIMAT0005238 | >dan-miR-275 | MIMAT0008451 |
| >ptc-miR478o | MIMAT0002089 | >smo-miR1087 | MIMAT0005239 | >dan-miR-317 | MIMAT0008452 |
| >ptc-miR478p | MIMAT0002090 | >smo-miR1088-5p | MIMAT0005240 | >dan-miR-309 | MIMAT0008453 |
| >ptc-miR478q | MIMAT0002091 | >smo-miR1088-3p | MIMAT0005241 | >dan-miR-279 | MIMAT0008454 |
| >ptc-miR478r | MIMAT0002092 | >smo-miR1089 | MIMAT0005242 | >dan-miR-276a | MIMAT0008455 |
| >ptc-miR478s | MIMAT0002093 | >smo-miR1090 | MIMAT0005243 | >dan-miR-98 | MIMAT0008456 |
| >ptc-miR478u | MIMAT0002094 | >smo-miR1091 | MIMAT0005244 | >dan-miR-5 | MIMAT0008457 |
| >ptc-miR479 | MIMAT0002095 | >smo-miR1092 | MIMAT0005245 | >dan-miR-9a | MIMAT0008458 |
| >ptc-miR480a | MIMAT0002096 | >smo-miR1093 | MIMAT0005246 | >dan-miR-315 | MIMAT0008459 |
| >ptc-miR480b | MIMAT0002097 | >smo-miR1094a | MIMAT0005247 | >dan-miR-14 | MIMAT0008460 |
| >ptc-miR481a | MIMAT0002098 | >smo-miR1094b | MIMAT0005248 | >dan-miR-33 | MIMAT0008461 |
| >ptc-miR481b | MIMAT0002099 | >smo-miR1094c | MIMAT0005249 | >dan-miR-6 | MIMAT0008462 |
| >ptc-miR481c | MIMAT0002100 | >smo-miR1095a | MIMAT0005250 | >dan-miR-318 | MIMAT0008463 |
| >ptc-miR481d | MIMAT0002101 | >smo-miR1095b | MIMAT0005251 | >dan-miR-305 | MIMAT0008464 |
| >ptc-miR481e | MIMAT0002102 | >smo-miR1096 | MIMAT0005252 | >dan-miR-314 | MIMAT0008465 |
| >ptc-miR482.2 | MIMAT0006785 | >smo-miR1097 | MIMAT0005253 | >dan-miR-289 | MIMAT0008466 |
| >ptc-miR482.1 | MIMAT0002103 | >smo-miR1098 | MIMAT0005254 | >dan-let-7 | MIMAT0008467 |
| >mmu-miR-463 | MIMAT0004758 | >smo-miR1099 | MIMAT0005255 | >dan-miR-133 | MIMAT0008468 |
| >mmu-miR-464 | MIMAT0002105 | >smo-miR1100 | MIMAT0005256 | >dan-miR-31b | MIMAT0008469 |
| >mmu-miR-465a-5p | MIMAT0002106 | >smo-miR1101-5p | MIMAT0005257 | >dan-miR-278 | MIMAT0008470 |
| >mmu-miR-465a-3p | MIMAT0004217 | >smo-miR1101-3p | MIMAT0005258 | >dan-miR-311a | MIMAT0008471 |
| >mmu-miR-466a-5p | MIMAT0004759 | >smo-miR1102 | MIMAT0005259 | >dan-miR-11 | MIMAT0008472 |
| >mmu-miR-466a-3p | MIMAT0002107 | >smo-miR319 | MIMAT0005260 | >dan-miR-125 | MIMAT0008473 |
| >mmu-miR-467a | MIMAT0003409 | >smo-miR1104 | MIMAT0005261 | >dan-miR-219 | MIMAT0008474 |
| >mmu-miR-468 | MIMAT0002109 | >smo-miR1105 | MIMAT0005262 | >dan-miR-311b | MIMAT0008475 |
| >mmu-miR-469 | MIMAT0002110 | >smo-miR1106 | MIMAT0005263 | >dan-miR-10 | MIMAT0008476 |
| >mmu-miR-470 | MIMAT0002111 | >smo-miR1107 | MIMAT0005264 | >dan-miR-31a | MIMAT0008477 |
| >mmu-miR-471 | MIMAT0002112 | >smo-miR1108 | MIMAT0005265 | >dan-miR-100 | MIMAT0008478 |
| >ath-miR447a | MIMAT0002113 | >smo-miR1109 | MIMAT0005266 | >der-miR-308 | MIMAT0008479 |
| >ath-miR447b | MIMAT0002114 | >smo-miR1110 | MIMAT0005267 | >der-miR-283 | MIMAT0008480 |
| >ath-miR447c | MIMAT0002115 | >smo-miR1111 | MIMAT0005268 | >der-miR-31b | MIMAT0008481 |
| >ssc-miR-105-1 | MIMAT0002116 | >smo-miR1112-5p | MIMAT0005269 | >der-miR-5 | MIMAT0008482 |
| >ssc-miR-105-2 | MIMAT0002117 | >smo-miR1112-3p | MIMAT0005270 | >der-miR-287 | MIMAT0008483 |
| >ssc-miR-106a | MIMAT0002118 | >smo-miR1113 | MIMAT0005271 | >der-miR-13b | MIMAT0008484 |
| >ssc-miR-122 | MIMAT0002119 | >smo-miR1114 | MIMAT0005272 | >der-miR-310b | MIMAT0008485 |
| >ssc-miR-125b | MIMAT0002120 | >smo-miR1115-5p | MIMAT0005273 | >der-let-7 | MIMAT0008486 |
| >ssc-miR-135 | MIMAT0002121 | >smo-miR1115-3p | MIMAT0005274 | >der-miR-309 | MIMAT0008487 |
| >ssc-miR-145 | MIMAT0002123 | >smo-miR1103-5p | MIMAT0005275 | >der-miR-100 | MIMAT0008488 |
| >ssc-miR-148a | MIMAT0002124 | >smo-miR1103-3p | MIMAT0005276 | >der-miR-311a | MIMAT0008489 |
| >ssc-miR-15b | MIMAT0002125 | >hiv1-miR-N367 | MIMAT0004478 | >der-miR-133 | MIMAT0008490 |
| >ssc-miR-181b | MIMAT0002126 | >hiv1-miR-H1 | MIMAT0004480 | >der-miR-286 | MIMAT0008491 |
| >ssc-miR-184 | MIMAT0002127 | >rno-miR-466b | MIMAT0005278 | >der-miR-280 | MIMAT0008492 |
| >ssc-miR-19a | MIMAT0002128 | >rno-miR-466c | MIMAT0005279 | >der-miR-92a | MIMAT0008493 |
| >ssc-miR-20 | MIMAT0002129 | >rno-miR-743b | MIMAT0005280 | >der-miR-306 | MIMAT0008494 |
| >ssc-miR-216 | MIMAT0002130 | >rno-miR-871 | MIMAT0005281 | >der-miR-316 | MIMAT0008496 |
| >ssc-miR-217 | MIMAT0002131 | >rno-miR-872 | MIMAT0005282 | >der-miR-307 | MIMAT0008497 |
| >ssc-miR-224 | MIMAT0002132 | >rno-miR-874 | MIMAT0005284 | >der-miR-14 | MIMAT0008498 |
| >ssc-miR-23a | MIMAT0002133 | >rno-miR-877 | MIMAT0005285 | >der-miR-8 | MIMAT0008499 |
| >ssc-miR-24 | MIMAT0002134 | >rno-miR-878 | MIMAT0005286 | >der-miR-11 | MIMAT0008500 |
| >ssc-miR-26a | MIMAT0002135 | >rno-miR-879 | MIMAT0005287 | >der-miR-281 | MIMAT0008502 |
| >ssc-miR-28 | MIMAT0002136 | >rno-miR-880 | MIMAT0005288 | >der-miR-275 | MIMAT0008503 |
| >ssc-miR-29b | MIMAT0002137 | >rno-miR-881 | MIMAT0005289 | >der-miR-6 | MIMAT0008504 |
| >ssc-miR-301 | MIMAT0002138 | >rno-miR-883 | MIMAT0005290 | >der-miR-9a | MIMAT0008505 |
| >ssc-miR-323 | MIMAT0002139 | >mmu-miR-582-5p | MIMAT0005291 | >der-miR-263a | MIMAT0008506 |
| >ssc-miR-326 | MIMAT0002140 | >mmu-miR-582-3p | MIMAT0005292 | >der-miR-9b | MIMAT0008507 |
| >ssc-miR-7 | MIMAT0002141 | >mmu-miR-467e | MIMAT0005293 | >der-miR-34 | MIMAT0008508 |
| >ssc-miR-95 | MIMAT0002142 | >rno-miR-147 | MIMAT0005297 | >der-miR-282 | MIMAT0008509 |
| >ssc-miR-140 | MIMAT0002143 | >rno-miR-17 | MIMAT0005298 | >der-miR-2a | MIMAT0008510 |
| >ssc-miR-181c | MIMAT0002144 | >rno-miR-181d | MIMAT0005299 | >der-miR-184 | MIMAT0008512 |
| >ssc-miR-183 | MIMAT0002145 | >rno-miR-182 | MIMAT0005300 | >der-miR-284 | MIMAT0008513 |
| >ssc-miR-205 | MIMAT0002146 | >rno-miR-188 | MIMAT0005301 | >der-miR-285 | MIMAT0008514 |
| >ssc-miR-214 | MIMAT0002147 | >rno-miR-190b | MIMAT0005302 | >der-miR-279 | MIMAT0008515 |
| >ssc-miR-27a | MIMAT0002148 | >rno-miR-196c | MIMAT0005303 | >der-miR-263b | MIMAT0008516 |
| >ssc-miR-32 | MIMAT0002149 | >rno-miR-301b | MIMAT0005304 | >der-miR-277 | MIMAT0008517 |
| >ssc-miR-325 | MIMAT0002150 | >rno-miR-375 | MIMAT0005307 | >der-miR-318 | MIMAT0008518 |
| >ssc-let-7c | MIMAT0002151 | >rno-miR-380 | MIMAT0005308 | >der-miR-31a | MIMAT0008519 |
| >ssc-let-7f | MIMAT0002152 | >rno-miR-384-5p | MIMAT0005309 | >der-miR-311b | MIMAT0008520 |
| >ssc-let-7i | MIMAT0002153 | >rno-miR-384-3p | MIMAT0005310 | >der-miR-274 | MIMAT0008521 |
| >ssc-miR-103 | MIMAT0002154 | >rno-miR-410 | MIMAT0005311 | >der-miR-311c | MIMAT0008522 |
| >ssc-miR-107 | MIMAT0002155 | >rno-miR-411 | MIMAT0005312 | >der-miR-iab-4-5p | MIMAT0008523 |
| >ssc-miR-124a | MIMAT0002156 | >rno-miR-423 | MIMAT0005313 | >der-miR-iab-4-3p | MIMAT0008524 |
| >ssc-miR-128 | MIMAT0002157 | >rno-miR-425 | MIMAT0005314 | >der-miR-79 | MIMAT0008525 |
| >ssc-miR-136 | MIMAT0002158 | >rno-miR-434 | MIMAT0005315 | >der-miR-125 | MIMAT0008526 |
| >ssc-miR-139 | MIMAT0002159 | >rno-miR-455 | MIMAT0005316 | >der-miR-305 | MIMAT0008527 |
| >ssc-miR-153 | MIMAT0002160 | >rno-miR-463 | MIMAT0005317 | >der-miR-314 | MIMAT0008528 |
| >ssc-miR-18 | MIMAT0002161 | >rno-miR-471 | MIMAT0005318 | >der-miR-276a | MIMAT0008529 |
| >ssc-miR-186 | MIMAT0002162 | >rno-miR-484 | MIMAT0005319 | >der-miR-278 | MIMAT0008530 |
| >ssc-miR-196 | MIMAT0002163 | >rno-miR-495 | MIMAT0005320 | >der-miR-12 | MIMAT0008531 |
| >ssc-miR-204 | MIMAT0002164 | >rno-miR-500 | MIMAT0005321 | >der-miR-4 | MIMAT0008532 |
| >ssc-miR-21 | MIMAT0002165 | >rno-miR-532-5p | MIMAT0005322 | >der-bantam | MIMAT0008533 |
| >ssc-miR-29c | MIMAT0002166 | >rno-miR-532-3p | MIMAT0005323 | >der-miR-1 | MIMAT0008534 |
| >ssc-miR-30c | MIMAT0002167 | >rno-miR-598-5p | MIMAT0005324 | >der-miR-2b | MIMAT0008535 |
| >ssc-miR-9-1 | MIMAT0002168 | >rno-miR-598-3p | MIMAT0005325 | >der-miR-312 | MIMAT0008536 |
| >ssc-miR-9-2 | MIMAT0002169 | >rno-miR-671 | MIMAT0005326 | >der-miR-7 | MIMAT0008537 |
| >hsa-miR-412 | MIMAT0002170 | >rno-miR-672 | MIMAT0005327 | >der-miR-3 | MIMAT0008538 |
| >hsa-miR-410 | MIMAT0002171 | >rno-miR-673 | MIMAT0005328 | >der-miR-315 | MIMAT0008539 |
| >hsa-miR-376b | MIMAT0002172 | >rno-miR-674-5p | MIMAT0005329 | >der-miR-2c | MIMAT0008540 |
| >hsa-miR-483-5p | MIMAT0004761 | >rno-miR-674-3p | MIMAT0005330 | >der-miR-317 | MIMAT0008541 |
| >hsa-miR-483-3p | MIMAT0002173 | >rno-miR-708 | MIMAT0005331 | >der-miR-276b | MIMAT0008542 |
| >hsa-miR-484 | MIMAT0002174 | >rno-miR-742 | MIMAT0005333 | >der-miR-92b | MIMAT0008543 |
| >hsa-miR-485-5p | MIMAT0002175 | >rno-miR-743a | MIMAT0005334 | >der-miR-87 | MIMAT0008544 |
| >hsa-miR-485-3p | MIMAT0002176 | >rno-miR-758 | MIMAT0005335 | >der-miR-33 | MIMAT0008545 |
| >hsa-miR-486-5p | MIMAT0002177 | >rno-miR-760-5p | MIMAT0005336 | >der-miR-210 | MIMAT0008546 |
| >hsa-miR-486-3p | MIMAT0004762 | >rno-miR-760-3p | MIMAT0005337 | >der-miR-124 | MIMAT0008547 |
| >hsa-miR-487a | MIMAT0002178 | >rno-miR-770 | MIMAT0005338 | >der-miR-304 | MIMAT0008548 |
| >kshv-miR-K12-10a | MIMAT0002179 | >rno-miR-873 | MIMAT0005339 | >der-miR-10 | MIMAT0008549 |
| >kshv-miR-K12-10b | MIMAT0002180 | >rno-miR-92b | MIMAT0005340 | >der-miR-13a | MIMAT0008550 |
| >kshv-miR-K12-11 | MIMAT0002181 | >rno-miR-488 | MIMAT0005341 | >der-miR-288 | MIMAT0008551 |
| >kshv-miR-K12-1 | MIMAT0002182 | >rno-miR-652 | MIMAT0005342 | >der-miR-219 | MIMAT0008552 |
| >kshv-miR-K12-2 | MIMAT0002183 | >tae-miR159a | MIMAT0005343 | >der-miR-310a | MIMAT0008553 |
| >kshv-miR-K12-9 | MIMAT0002185 | >tae-miR159b | MIMAT0005344 | >der-miR-9c | MIMAT0008554 |
| >kshv-miR-K12-8 | MIMAT0002186 | >tae-miR160 | MIMAT0005345 | >der-miR-289 | MIMAT0008555 |
| >kshv-miR-K12-7 | MIMAT0002187 | >tae-miR164 | MIMAT0005346 | >dgr-miR-277 | MIMAT0008556 |
| >kshv-miR-K12-6-5p | MIMAT0002188 | >tae-miR167 | MIMAT0005347 | >dgr-miR-100 | MIMAT0008557 |
| >kshv-miR-K12-6-3p | MIMAT0002189 | >tae-miR171 | MIMAT0005348 | >dgr-miR-305 | MIMAT0008558 |
| >kshv-miR-K12-5 | MIMAT0002190 | >tae-miR399 | MIMAT0005349 | >dgr-bantam | MIMAT0008559 |
| >kshv-miR-K12-4-5p | MIMAT0002191 | >tae-miR408 | MIMAT0005350 | >dgr-miR-12 | MIMAT0008560 |
| >kshv-miR-K12-4-3p | MIMAT0002192 | >tae-miR444 | MIMAT0005351 | >dgr-miR-219 | MIMAT0008561 |
| >kshv-miR-K12-3 | MIMAT0002193 | >tae-miR1117 | MIMAT0005352 | >dgr-miR-307 | MIMAT0008562 |
| >mml-miR-200c | MIMAT0002195 | >tae-miR1118 | MIMAT0005353 | >dgr-miR-287 | MIMAT0008563 |
| >mml-miR-141 | MIMAT0002196 | >tae-miR1119 | MIMAT0005354 | >dgr-miR-263b | MIMAT0008564 |
| >ggo-miR-200c | MIMAT0002197 | >tae-miR1120 | MIMAT0005355 | >dgr-miR-308 | MIMAT0008565 |
| >ggo-miR-141 | MIMAT0002198 | >tae-miR1121 | MIMAT0005356 | >dgr-miR-184 | MIMAT0008567 |
| >ppy-miR-200c | MIMAT0002199 | >tae-miR1122 | MIMAT0005357 | >dgr-miR-2b | MIMAT0008568 |
| >ppy-miR-141 | MIMAT0002200 | >tae-miR1123 | MIMAT0005358 | >dgr-miR-6 | MIMAT0008569 |
| >ppa-miR-141 | MIMAT0002201 | >tae-miR1124 | MIMAT0005359 | >dgr-miR-5 | MIMAT0008570 |
| >ggo-miR-15b | MIMAT0002202 | >tae-miR1125 | MIMAT0005360 | >dgr-miR-92a | MIMAT0008571 |
| >age-miR-15b | MIMAT0002203 | >tae-miR1126 | MIMAT0005361 | >dgr-miR-11 | MIMAT0008572 |
| >ppa-miR-15b | MIMAT0002204 | >tae-miR1127 | MIMAT0005362 | >dgr-miR-276b | MIMAT0008574 |
| >ppy-miR-15b | MIMAT0002205 | >tae-miR1128 | MIMAT0005363 | >dgr-miR-276a | MIMAT0008576 |
| >ptr-miR-15b | MIMAT0002206 | >tae-miR1129 | MIMAT0005364 | >dgr-miR-309 | MIMAT0008577 |
| >mml-miR-15b | MIMAT0002207 | >tae-miR1130 | MIMAT0005365 | >dgr-miR-iab-4-5p | MIMAT0008578 |
| >lla-miR-15b | MIMAT0002208 | >tae-miR1131 | MIMAT0005366 | >dgr-miR-iab-4-3p | MIMAT0008579 |
| >mne-miR-15b | MIMAT0002209 | >tae-miR1132 | MIMAT0005367 | >dgr-miR-3 | MIMAT0008580 |
| >ptr-miR-23b | MIMAT0002210 | >tae-miR1133 | MIMAT0005368 | >dgr-miR-285 | MIMAT0008581 |
| >ppy-miR-23b | MIMAT0002211 | >tae-miR1134 | MIMAT0005369 | >dgr-miR-9b | MIMAT0008582 |
| >ppa-miR-23b | MIMAT0002212 | >tae-miR1135 | MIMAT0005370 | >dgr-miR-79 | MIMAT0008583 |
| >mml-miR-30b | MIMAT0002213 | >tae-miR1136 | MIMAT0005371 | >dgr-miR-318 | MIMAT0008584 |
| >ptr-miR-30b | MIMAT0002214 | >tae-miR1137 | MIMAT0005372 | >dgr-miR-31a | MIMAT0008585 |
| >ggo-miR-30b | MIMAT0002215 | >tae-miR1138 | MIMAT0005373 | >dgr-miR-317 | MIMAT0008586 |
| >lla-miR-30b | MIMAT0002216 | >tae-miR1139 | MIMAT0005374 | >dgr-miR-281 | MIMAT0008588 |
| >mne-miR-30b | MIMAT0002217 | >ppt-miR477b | MIMAT0005375 | >dgr-miR-9a | MIMAT0008589 |
| >age-miR-30b | MIMAT0002218 | >cre-miR1142 | MIMAT0005376 | >dgr-miR-286 | MIMAT0008590 |
| >ppa-miR-30b | MIMAT0002219 | >cre-miR1143 | MIMAT0005378 | >dgr-miR-316 | MIMAT0008591 |
| >ggo-miR-125b | MIMAT0002220 | >cre-miR1144a.2 | MIMAT0005379 | >dgr-miR-306 | MIMAT0008592 |
| >age-miR-125b | MIMAT0002221 | >cre-miR1144a.1 | MIMAT0005380 | >dgr-miR-8 | MIMAT0008593 |
| >ppa-miR-125b | MIMAT0002222 | >cre-miR1145.2 | MIMAT0005381 | >dgr-miR-4 | MIMAT0008594 |
| >ppy-miR-125b | MIMAT0002223 | >cre-miR1145.1 | MIMAT0005382 | >dgr-miR-304 | MIMAT0008595 |
| >ptr-miR-125b | MIMAT0002224 | >cre-miR1146 | MIMAT0005383 | >dgr-let-7 | MIMAT0008596 |
| >mml-miR-125b | MIMAT0002225 | >cre-miR1147.1 | MIMAT0005384 | >dgr-miR-278 | MIMAT0008597 |
| >sla-miR-125b | MIMAT0002226 | >cre-miR1147.2 | MIMAT0005385 | >dgr-miR-314 | MIMAT0008598 |
| >lla-miR-125b | MIMAT0002227 | >cre-miR1148.1 | MIMAT0005386 | >dgr-miR-279 | MIMAT0008599 |
| >mne-miR-125b | MIMAT0002228 | >cre-miR1148.2 | MIMAT0005387 | >dgr-miR-288 | MIMAT0008600 |
| >mml-miR-128a | MIMAT0002229 | >cre-miR1149.1 | MIMAT0005388 | >dgr-miR-10 | MIMAT0008601 |
| >ptr-miR-128 | MIMAT0002230 | >cre-miR1149.2 | MIMAT0005389 | >dgr-miR-14 | MIMAT0008602 |
| >ppy-miR-128 | MIMAT0002231 | >cre-miR1150.3 | MIMAT0005390 | >dgr-miR-33 | MIMAT0008603 |
| >sla-miR-128 | MIMAT0002232 | >cre-miR1150.2 | MIMAT0005391 | >dgr-miR-315 | MIMAT0008604 |
| >age-miR-128 | MIMAT0002233 | >cre-miR1150.1 | MIMAT0005392 | >dgr-miR-13b | MIMAT0008605 |
| >ppa-miR-128 | MIMAT0002234 | >cre-miR1151a | MIMAT0005393 | >dgr-miR-9c | MIMAT0008606 |
| >mml-miR-130a | MIMAT0002235 | >cre-miR1151b | MIMAT0005395 | >dgr-miR-2a | MIMAT0008607 |
| >ggo-miR-130a | MIMAT0002236 | >cre-miR1152 | MIMAT0005397 | >dgr-miR-87 | MIMAT0008608 |
| >mne-miR-130a | MIMAT0002237 | >cre-miR1153.1 | MIMAT0005398 | >dgr-miR-1 | MIMAT0008609 |
| >ppa-miR-130a | MIMAT0002238 | >cre-miR1153.2 | MIMAT0005400 | >dgr-miR-125 | MIMAT0008610 |
| >ggo-miR-133a | MIMAT0002239 | >cre-miR1154 | MIMAT0005402 | >dgr-miR-284 | MIMAT0008611 |
| >age-miR-133a | MIMAT0002240 | >cre-miR1155 | MIMAT0005404 | >dgr-miR-31b | MIMAT0008612 |
| >ppa-miR-133a | MIMAT0002241 | >cre-miR1156.1 | MIMAT0005405 | >dgr-miR-210 | MIMAT0008613 |
| >ppy-miR-133a | MIMAT0002242 | >cre-miR1156.2 | MIMAT0005406 | >dgr-miR-282 | MIMAT0008614 |
| >ptr-miR-133a | MIMAT0002243 | >cre-miR1157 | MIMAT0005408 | >dgr-miR-124 | MIMAT0008615 |
| >mml-miR-133a | MIMAT0002244 | >cre-miR1158 | MIMAT0005409 | >dgr-miR-280 | MIMAT0008616 |
| >sla-miR-133a | MIMAT0002245 | >cre-miR1160.2 | MIMAT0005410 | >dgr-miR-13a | MIMAT0008617 |
| >lla-miR-133a | MIMAT0002246 | >cre-miR1160.1 | MIMAT0005411 | >dgr-miR-283 | MIMAT0008618 |
| >mne-miR-133a | MIMAT0002247 | >cre-miR1160.3 | MIMAT0005412 | >dgr-miR-34 | MIMAT0008619 |
| >lla-miR-135 | MIMAT0002248 | >cre-miR1161 | MIMAT0005413 | >dgr-miR-133 | MIMAT0008620 |
| >age-miR-135 | MIMAT0002249 | >cre-miR1162 | MIMAT0005416 | >dgr-miR-263a | MIMAT0008621 |
| >ppa-miR-135 | MIMAT0002250 | >cre-miR1163.2 | MIMAT0005417 | >dgr-miR-7 | MIMAT0008622 |
| >mml-miR-135a | MIMAT0002251 | >cre-miR1163.1 | MIMAT0005418 | >dgr-miR-274 | MIMAT0008623 |
| >ptr-miR-135a | MIMAT0002252 | >cre-miR1164 | MIMAT0005419 | >dgr-miR-92b | MIMAT0008624 |
| >ggo-miR-135 | MIMAT0002253 | >cre-miR1165 | MIMAT0005420 | >dgr-miR-275 | MIMAT0008625 |
| >ppy-miR-135 | MIMAT0002254 | >cre-miR1166.1 | MIMAT0005422 | >dgr-miR-2c | MIMAT0008626 |
| >ptr-miR-140 | MIMAT0002255 | >cre-miR1166.2 | MIMAT0005423 | >dmo-miR-316 | MIMAT0008627 |
| >mne-miR-140 | MIMAT0002256 | >cre-miR1168.1 | MIMAT0005424 | >dmo-miR-284 | MIMAT0008628 |
| >ptr-miR-143 | MIMAT0002257 | >cre-miR1168.2 | MIMAT0005425 | >dmo-miR-4 | MIMAT0008629 |
| >ggo-miR-143 | MIMAT0002258 | >cre-miR1169 | MIMAT0005427 | >dmo-miR-12 | MIMAT0008630 |
| >ppy-miR-143 | MIMAT0002259 | >cre-miR1170.2 | MIMAT0005428 | >dmo-miR-276b | MIMAT0008631 |
| >lla-miR-143 | MIMAT0002260 | >cre-miR1170.1 | MIMAT0005429 | >dmo-miR-14 | MIMAT0008632 |
| >ppa-miR-143 | MIMAT0002261 | >cre-miR1171 | MIMAT0005430 | >dmo-miR-92a | MIMAT0008633 |
| >ptr-miR-144 | MIMAT0002262 | >cre-miR1173 | MIMAT0005431 | >dmo-bantam | MIMAT0008634 |
| >ppy-miR-144 | MIMAT0002263 | >cre-miR1172.2 | MIMAT0005432 | >dmo-miR-6 | MIMAT0008635 |
| >mne-miR-144 | MIMAT0002264 | >cre-miR1172.1 | MIMAT0005433 | >dmo-miR-13b | MIMAT0008636 |
| >ppa-miR-144 | MIMAT0002265 | >cre-miR1167 | MIMAT0005434 | >dmo-miR-100 | MIMAT0008637 |
| >mml-miR-145 | MIMAT0002266 | >cre-miR1144b | MIMAT0005435 | >dmo-miR-219 | MIMAT0008638 |
| >ptr-miR-145 | MIMAT0002267 | >cre-miR1159.2 | MIMAT0005436 | >dmo-miR-31b | MIMAT0008639 |
| >ggo-miR-145 | MIMAT0002268 | >cre-miR1159.1 | MIMAT0005437 | >dmo-miR-283 | MIMAT0008640 |
| >ppy-miR-145 | MIMAT0002269 | >aga-miR-1174 | MIMAT0005524 | >dmo-miR-281 | MIMAT0008642 |
| >mne-miR-145 | MIMAT0002270 | >aga-miR-1175 | MIMAT0005525 | >dmo-miR-286 | MIMAT0008643 |
| >mml-miR-153 | MIMAT0002271 | >aga-miR-34 | MIMAT0005526 | >dmo-miR-314 | MIMAT0008644 |
| >ppy-miR-153 | MIMAT0002272 | >aga-miR-12 | MIMAT0005527 | >dmo-miR-282 | MIMAT0008645 |
| >mne-miR-153 | MIMAT0002273 | >aga-miR-996 | MIMAT0005528 | >dmo-miR-305 | MIMAT0008646 |
| >ggo-miR-153 | MIMAT0002274 | >aga-miR-989 | MIMAT0005529 | >dmo-miR-8 | MIMAT0008647 |
| >ptr-miR-9 | MIMAT0002275 | >aga-miR-306 | MIMAT0005530 | >dmo-miR-306 | MIMAT0008648 |
| >ggo-miR-9 | MIMAT0002276 | >ddi-miR-1176 | MIMAT0005531 | >dmo-miR-2b | MIMAT0008650 |
| >lla-miR-9 | MIMAT0002277 | >ddi-miR-1177 | MIMAT0005532 | >dmo-miR-285 | MIMAT0008651 |
| >mne-miR-9 | MIMAT0002278 | >mcmv-miR-m01-1 | MIMAT0005533 | >dmo-miR-2c | MIMAT0008652 |
| >age-miR-9 | MIMAT0002279 | >mcmv-miR-m01-2 | MIMAT0005534 | >dmo-miR-263a | MIMAT0008653 |
| >lca-miR-125b | MIMAT0002280 | >mcmv-miR-m01-3 | MIMAT0005536 | >dmo-miR-276a | MIMAT0008654 |
| >mml-miR-127 | MIMAT0002281 | >mcmv-miR-m01-4 | MIMAT0005538 | >dmo-miR-274 | MIMAT0008655 |
| >ptr-miR-127 | MIMAT0002282 | >mcmv-miR-m21-1 | MIMAT0005540 | >dmo-miR-34 | MIMAT0008656 |
| >ppy-miR-127 | MIMAT0002283 | >mcmv-miR-m22-1 | MIMAT0005541 | >dmo-miR-304 | MIMAT0008657 |
| >sla-miR-127 | MIMAT0002284 | >mcmv-miR-M23-1-5p | MIMAT0005542 | >dmo-miR-92b | MIMAT0008658 |
| >lla-miR-127 | MIMAT0002285 | >mcmv-miR-M23-1-3p | MIMAT0005543 | >dmo-miR-287 | MIMAT0008659 |
| >mne-miR-127 | MIMAT0002286 | >mcmv-miR-M23-2 | MIMAT0005545 | >dmo-miR-309 | MIMAT0008660 |
| >age-miR-127 | MIMAT0002287 | >mcmv-miR-M44-1 | MIMAT0005546 | >dmo-miR-13a | MIMAT0008661 |
| >ggo-miR-134 | MIMAT0002288 | >mcmv-miR-M55-1 | MIMAT0005547 | >dmo-miR-87 | MIMAT0008662 |
| >ppy-miR-134 | MIMAT0002289 | >mcmv-miR-m59-1 | MIMAT0005548 | >dmo-miR-280 | MIMAT0008663 |
| >mne-miR-134 | MIMAT0002290 | >mcmv-miR-m59-2 | MIMAT0005549 | >dmo-miR-275 | MIMAT0008664 |
| >ppa-miR-134 | MIMAT0002291 | >mcmv-miR-M87-1 | MIMAT0005550 | >dmo-miR-307 | MIMAT0008665 |
| >ptr-miR-136 | MIMAT0002292 | >mcmv-miR-m88-1 | MIMAT0005552 | >dmo-miR-9b | MIMAT0008666 |
| >ggo-miR-136 | MIMAT0002293 | >mcmv-miR-M95-1-5p | MIMAT0005553 | >dmo-miR-210 | MIMAT0008667 |
| >ppy-miR-136 | MIMAT0002294 | >mcmv-miR-M95-1-3p | MIMAT0005554 | >dmo-miR-3 | MIMAT0008668 |
| >ppa-miR-136 | MIMAT0002295 | >mcmv-miR-m107-1-5p | MIMAT0005555 | >dmo-miR-125 | MIMAT0008669 |
| >ptr-miR-154 | MIMAT0002296 | >mcmv-miR-m107-1-3p | MIMAT0005556 | >dmo-miR-9a | MIMAT0008670 |
| >ggo-miR-154 | MIMAT0002297 | >mcmv-miR-m108-1 | MIMAT0005558 | >dmo-miR-317 | MIMAT0008671 |
| >ppy-miR-154 | MIMAT0002298 | >mcmv-miR-m108-2-5p.2 | MIMAT0005559 | >dmo-miR-2a | MIMAT0008672 |
| >mne-miR-154 | MIMAT0002299 | >mcmv-miR-m108-2-5p.1 | MIMAT0005560 | >dmo-miR-184 | MIMAT0008674 |
| >ppa-miR-154 | MIMAT0002300 | >mcmv-miR-m108-2-3p | MIMAT0005561 | >dmo-miR-288 | MIMAT0008675 |
| >ptr-miR-184 | MIMAT0002301 | >xtr-miR-31b | MIMAT0005562 | >dmo-miR-278 | MIMAT0008676 |
| >ppy-miR-184 | MIMAT0002302 | >xtr-miR-146b | MIMAT0005563 | >dmo-miR-31a | MIMAT0008677 |
| >mne-miR-184 | MIMAT0002303 | >xtr-miR-320 | MIMAT0005564 | >dmo-miR-133 | MIMAT0008678 |
| >ptr-miR-186 | MIMAT0002304 | >xtr-miR-375 | MIMAT0005565 | >dmo-miR-11 | MIMAT0008679 |
| >ggo-miR-186 | MIMAT0002305 | >xtr-miR-427 | MIMAT0005566 | >dmo-miR-iab-4-5p | MIMAT0008680 |
| >ppa-miR-186 | MIMAT0002306 | >hsa-miR-1178 | MIMAT0005823 | >dmo-miR-iab-4-3p | MIMAT0008681 |
| >mml-miR-188 | MIMAT0002307 | >hsa-miR-1179 | MIMAT0005824 | >dmo-miR-279 | MIMAT0008682 |
| >ptr-miR-188 | MIMAT0002308 | >hsa-miR-1180 | MIMAT0005825 | >dmo-miR-9c | MIMAT0008683 |
| >ppy-miR-188 | MIMAT0002309 | >hsa-miR-1181 | MIMAT0005826 | >dmo-miR-318 | MIMAT0008684 |
| >mne-miR-188 | MIMAT0002310 | >hsa-miR-1182 | MIMAT0005827 | >dmo-miR-277 | MIMAT0008685 |
| >ppa-miR-188 | MIMAT0002311 | >hsa-miR-1183 | MIMAT0005828 | >dmo-let-7 | MIMAT0008686 |
| >mml-miR-190a | MIMAT0002312 | >hsa-miR-1184 | MIMAT0005829 | >dmo-miR-315 | MIMAT0008687 |
| >ptr-miR-190a | MIMAT0002313 | >mmu-miR-4661 | MIMAT0005830 | >dmo-miR-124 | MIMAT0008688 |
| >ggo-miR-190 | MIMAT0002314 | >mmu-miR-669k | MIMAT0005831 | >dmo-miR-308 | MIMAT0008689 |
| >ppa-miR-190 | MIMAT0002315 | >mmu-miR-669g | MIMAT0005832 | >dmo-miR-79 | MIMAT0008690 |
| >ggo-miR-195 | MIMAT0002316 | >mmu-miR-669d | MIMAT0005833 | >dmo-miR-5 | MIMAT0008691 |
| >ppa-miR-195 | MIMAT0002317 | >mmu-miR-466i | MIMAT0005834 | >dmo-miR-7 | MIMAT0008692 |
| >ppy-miR-206 | MIMAT0002318 | >mmu-miR-1-2-as | MIMAT0005835 | >dmo-miR-10 | MIMAT0008693 |
| >mne-miR-206 | MIMAT0002319 | >mmu-miR-1186 | MIMAT0005836 | >dmo-miR-1 | MIMAT0008694 |
| >mml-miR-21 | MIMAT0002320 | >mmu-miR-1187 | MIMAT0005837 | >dmo-miR-33 | MIMAT0008695 |
| >ptr-miR-21 | MIMAT0002321 | >mmu-miR-669j | MIMAT0005838 | >dmo-miR-313 | MIMAT0008696 |
| >ggo-miR-21 | MIMAT0002322 | >mmu-miR-669f | MIMAT0005839 | >dpe-miR-307 | MIMAT0008697 |
| >ppy-miR-21 | MIMAT0002323 | >mmu-miR-669i | MIMAT0005840 | >dpe-miR-3 | MIMAT0008698 |
| >mne-miR-21 | MIMAT0002324 | >mmu-miR-669h-5p | MIMAT0005841 | >dpe-let-7 | MIMAT0008699 |
| >age-miR-21 | MIMAT0002325 | >mmu-miR-669h-3p | MIMAT0005842 | >dpe-miR-184 | MIMAT0008701 |
| >ppa-miR-21 | MIMAT0002326 | >mmu-miR-1188 | MIMAT0005843 | >dpe-miR-277 | MIMAT0008702 |
| >age-miR-22 | MIMAT0002327 | >mmu-miR-466f | MIMAT0005844 | >dpe-miR-14 | MIMAT0008703 |
| >ppa-miR-22 | MIMAT0002328 | >mmu-miR-466k | MIMAT0005845 | >dpe-miR-210 | MIMAT0008704 |
| >lca-miR-22 | MIMAT0002329 | >mmu-miR-467f | MIMAT0005846 | >dpe-miR-276a | MIMAT0008705 |
| >mml-miR-22 | MIMAT0002330 | >mmu-miR-1190 | MIMAT0005847 | >dpe-miR-13a | MIMAT0008706 |
| >ppy-miR-22 | MIMAT0002331 | >mmu-miR-466j | MIMAT0005848 | >dpe-miR-6 | MIMAT0008707 |
| >ptr-miR-22 | MIMAT0002332 | >mmu-miR-1191 | MIMAT0005849 | >dpe-miR-284 | MIMAT0008708 |
| >sla-miR-22 | MIMAT0002333 | >mmu-miR-1192 | MIMAT0005850 | >dpe-miR-286 | MIMAT0008709 |
| >lla-miR-22 | MIMAT0002334 | >mmu-miR-1193 | MIMAT0005851 | >dpe-miR-8 | MIMAT0008710 |
| >mne-miR-22 | MIMAT0002335 | >mmu-miR-1194 | MIMAT0005852 | >dpe-miR-13b | MIMAT0008711 |
| >mml-miR-189 | MIMAT0002336 | >mmu-miR-669e | MIMAT0005853 | >dpe-miR-133 | MIMAT0008712 |
| >mml-miR-24 | MIMAT0002337 | >mmu-miR-467g | MIMAT0005854 | >dpe-miR-283 | MIMAT0008713 |
| >ppy-miR-189 | MIMAT0002338 | >mmu-miR-467h | MIMAT0005855 | >dpe-miR-5 | MIMAT0008714 |
| >ppy-miR-24 | MIMAT0002339 | >mmu-miR-1195 | MIMAT0005856 | >dpe-miR-34 | MIMAT0008715 |
| >mne-miR-189 | MIMAT0002340 | >mmu-miR-1196 | MIMAT0005857 | >dpe-miR-282 | MIMAT0008716 |
| >mne-miR-24 | MIMAT0002341 | >mmu-miR-1197 | MIMAT0005858 | >dpe-miR-9a | MIMAT0008717 |
| >ppa-miR-189 | MIMAT0002342 | >mmu-miR-1198 | MIMAT0005859 | >dpe-miR-100 | MIMAT0008718 |
| >ppa-miR-24 | MIMAT0002343 | >mmu-miR-1199 | MIMAT0005860 | >dpe-miR-219 | MIMAT0008719 |
| >ptr-miR-26a | MIMAT0002344 | >cpa-miR162a | MIMAT0005567 | >dpe-miR-314 | MIMAT0008720 |
| >ggo-miR-26a | MIMAT0002345 | >ptr-miR-1224-5p | MIMAT0005568 | >dpe-bantam | MIMAT0008721 |
| >ppy-miR-26a | MIMAT0002346 | >ptr-miR-1224-3p | MIMAT0005569 | >dpe-miR-280 | MIMAT0008722 |
| >lla-miR-26a | MIMAT0002347 | >mml-miR-1224 | MIMAT0005570 | >dpe-miR-31a | MIMAT0008723 |
| >mne-miR-26a | MIMAT0002348 | >hsa-miR-1225-5p | MIMAT0005572 | >dpe-miR-1 | MIMAT0008724 |
| >mml-miR-26a | MIMAT0002349 | >hsa-miR-1225-3p | MIMAT0005573 | >dpe-miR-125 | MIMAT0008725 |
| >ppa-miR-26a | MIMAT0002350 | >mml-miR-1225-5p | MIMAT0005574 | >dpe-miR-278 | MIMAT0008726 |
| >age-miR-28 | MIMAT0002351 | >mml-miR-1225-3p | MIMAT0005575 | >dpe-miR-281 | MIMAT0008728 |
| >mml-miR-28 | MIMAT0002352 | >hsa-miR-1226 | MIMAT0005577 | >dpe-miR-2a | MIMAT0008729 |
| >ptr-miR-28 | MIMAT0002353 | >ptr-miR-1226 | MIMAT0005578 | >dpe-miR-315 | MIMAT0008730 |
| >ggo-miR-28 | MIMAT0002354 | >mml-miR-1226 | MIMAT0005579 | >dpe-miR-124 | MIMAT0008731 |
| >ppy-miR-28 | MIMAT0002355 | >hsa-miR-1227 | MIMAT0005580 | >dpe-miR-308 | MIMAT0008732 |
| >mne-miR-28 | MIMAT0002356 | >mml-miR-1227 | MIMAT0005581 | >dpe-miR-87 | MIMAT0008733 |
| >sla-miR-28 | MIMAT0002357 | >hsa-miR-1228 | MIMAT0005583 | >dpe-miR-279 | MIMAT0008734 |
| >lla-miR-28 | MIMAT0002358 | >hsa-miR-1229 | MIMAT0005584 | >dpe-miR-263b | MIMAT0008735 |
| >ppa-miR-28 | MIMAT0002359 | >mml-miR-1230 | MIMAT0005585 | >dpe-miR-2c | MIMAT0008736 |
| >ggo-miR-29a | MIMAT0002360 | >hsa-miR-1231 | MIMAT0005586 | >dpe-miR-11 | MIMAT0008737 |
| >age-miR-29a | MIMAT0002361 | >mml-miR-1232 | MIMAT0005587 | >dpe-miR-2b | MIMAT0008738 |
| >ppa-miR-29a | MIMAT0002362 | >hsa-miR-1233 | MIMAT0005588 | >dpe-miR-317 | MIMAT0008739 |
| >ppy-miR-29a | MIMAT0002363 | >hsa-miR-1234 | MIMAT0005589 | >dpe-miR-iab-4-5p | MIMAT0008740 |
| >ptr-miR-29a | MIMAT0002364 | >mml-miR-1235 | MIMAT0005590 | >dpe-miR-iab-4-3p | MIMAT0008741 |
| >mml-miR-29a | MIMAT0002365 | >hsa-miR-1236 | MIMAT0005591 | >dpe-miR-7 | MIMAT0008742 |
| >sla-miR-29a | MIMAT0002366 | >hsa-miR-1237 | MIMAT0005592 | >dpe-miR-33 | MIMAT0008743 |
| >lla-miR-29a | MIMAT0002367 | >hsa-miR-1238 | MIMAT0005593 | >dpe-miR-275 | MIMAT0008744 |
| >mne-miR-29a | MIMAT0002368 | >mml-miR-1239 | MIMAT0005594 | >dpe-miR-9b | MIMAT0008745 |
| >mml-miR-30a-5p | MIMAT0002369 | >mml-miR-1240 | MIMAT0005861 | >dpe-miR-9c | MIMAT0008746 |
| >mml-miR-30a-3p | MIMAT0002370 | >mml-miR-1241 | MIMAT0005862 | >dpe-miR-79 | MIMAT0008747 |
| >ptr-miR-30a-5p | MIMAT0002371 | >hsa-miR-1200 | MIMAT0005863 | >dpe-miR-289 | MIMAT0008748 |
| >ptr-miR-30a-3p | MIMAT0002372 | >hsa-miR-1201 | MIMAT0005864 | >dpe-miR-318 | MIMAT0008749 |
| >ggo-miR-30a-5p | MIMAT0002373 | >hsa-miR-1202 | MIMAT0005865 | >dpe-miR-276b | MIMAT0008750 |
| >ggo-miR-30a-3p | MIMAT0002374 | >hsa-miR-1203 | MIMAT0005866 | >dpe-miR-4 | MIMAT0008751 |
| >ppy-miR-30a-5p | MIMAT0002375 | >hsa-miR-663b | MIMAT0005867 | >dpe-miR-31b | MIMAT0008752 |
| >ppy-miR-30a-3p | MIMAT0002376 | >hsa-miR-1204 | MIMAT0005868 | >dpe-miR-288 | MIMAT0008753 |
| >ppa-miR-30a-5p | MIMAT0002377 | >hsa-miR-1205 | MIMAT0005869 | >dpe-miR-10 | MIMAT0008754 |
| >ppa-miR-30a-3p | MIMAT0002378 | >hsa-miR-1206 | MIMAT0005870 | >dpe-miR-305 | MIMAT0008755 |
| >mml-miR-31 | MIMAT0002379 | >hsa-miR-1207-5p | MIMAT0005871 | >dpe-miR-92b | MIMAT0008756 |
| >ptr-miR-31 | MIMAT0002380 | >hsa-miR-1207-3p | MIMAT0005872 | >dpe-miR-287 | MIMAT0008757 |
| >ggo-miR-31 | MIMAT0002381 | >hsa-miR-1208 | MIMAT0005873 | >dpe-miR-274 | MIMAT0008758 |
| >ppy-miR-31 | MIMAT0002382 | >rno-miR-146b | MIMAT0005595 | >dpe-miR-306 | MIMAT0008759 |
| >mne-miR-31 | MIMAT0002383 | >rno-miR-551b | MIMAT0005596 | >dpe-miR-309 | MIMAT0008760 |
| >ppa-miR-31 | MIMAT0002384 | >hsa-miR-548e | MIMAT0005874 | >dpe-miR-263a | MIMAT0008761 |
| >mml-miR-32 | MIMAT0002385 | >hsa-miR-548j | MIMAT0005875 | >dpe-miR-12 | MIMAT0008762 |
| >ptr-miR-32 | MIMAT0002386 | >hsa-miR-1285 | MIMAT0005876 | >dpe-miR-92a | MIMAT0008763 |
| >ggo-miR-32 | MIMAT0002387 | >hsa-miR-1286 | MIMAT0005877 | >dpe-miR-316 | MIMAT0008764 |
| >ppy-miR-32 | MIMAT0002388 | >hsa-miR-1287 | MIMAT0005878 | >dse-miR-6 | MIMAT0008765 |
| >sla-miR-32 | MIMAT0002389 | >hsa-miR-1289 | MIMAT0005879 | >dse-miR-12 | MIMAT0008766 |
| >mne-miR-32 | MIMAT0002390 | >hsa-miR-1290 | MIMAT0005880 | >dse-miR-280 | MIMAT0008767 |
| >ppa-miR-32 | MIMAT0002391 | >hsa-miR-1291 | MIMAT0005881 | >dse-miR-263a | MIMAT0008768 |
| >mml-miR-33a | MIMAT0002392 | >hsa-miR-548k | MIMAT0005882 | >dse-miR-79 | MIMAT0008769 |
| >ptr-miR-33a | MIMAT0002393 | >hsa-miR-1293 | MIMAT0005883 | >dse-miR-287 | MIMAT0008770 |
| >ggo-miR-33 | MIMAT0002394 | >hsa-miR-1294 | MIMAT0005884 | >dse-miR-7 | MIMAT0008771 |
| >ppy-miR-33 | MIMAT0002395 | >hsa-miR-1295 | MIMAT0005885 | >dse-miR-8 | MIMAT0008772 |
| >mne-miR-33 | MIMAT0002396 | >hsa-miR-1297 | MIMAT0005886 | >dse-miR-87 | MIMAT0008773 |
| >ppa-miR-33 | MIMAT0002397 | >hsa-miR-1299 | MIMAT0005887 | >dse-miR-92b | MIMAT0008774 |
| >ptr-miR-95 | MIMAT0002398 | >hsa-miR-1300 | MIMAT0005888 | >dse-bantam | MIMAT0008775 |
| >ggo-miR-95 | MIMAT0002399 | >hsa-miR-5481 | MIMAT0005889 | >dse-miR-210 | MIMAT0008776 |
| >ppy-miR-95 | MIMAT0002400 | >hsa-miR-1302 | MIMAT0005890 | >dse-miR-2c | MIMAT0008777 |
| >sla-miR-95 | MIMAT0002401 | >hsa-miR-1303 | MIMAT0005891 | >dse-miR-276b | MIMAT0008778 |
| >lla-miR-95 | MIMAT0002402 | >hsa-miR-1304 | MIMAT0005892 | >dse-miR-310 | MIMAT0008779 |
| >ppa-miR-95 | MIMAT0002403 | >hsa-miR-1305 | MIMAT0005893 | >dse-miR-2b | MIMAT0008780 |
| >mml-miR-98 | MIMAT0002404 | >hsa-miR-1243 | MIMAT0005894 | >dse-miR-278 | MIMAT0008781 |
| >ptr-miR-98 | MIMAT0002405 | >hsa-miR-548f | MIMAT0005895 | >dse-miR-281 | MIMAT0008783 |
| >ggo-miR-98 | MIMAT0002406 | >hsa-miR-1244 | MIMAT0005896 | >dse-miR-10 | MIMAT0008784 |
| >ppy-miR-98 | MIMAT0002407 | >hsa-miR-1245 | MIMAT0005897 | >dse-miR-92a | MIMAT0008785 |
| >age-miR-98 | MIMAT0002408 | >hsa-miR-1246 | MIMAT0005898 | >dse-miR-13a | MIMAT0008786 |
| >ppa-miR-98 | MIMAT0002409 | >hsa-miR-1247 | MIMAT0005899 | >dse-miR-34 | MIMAT0008787 |
| >mml-miR-99a | MIMAT0002410 | >hsa-miR-1248 | MIMAT0005900 | >dse-miR-11 | MIMAT0008788 |
| >ptr-miR-99a | MIMAT0002411 | >hsa-miR-1249 | MIMAT0005901 | >dse-miR-317 | MIMAT0008789 |
| >ggo-miR-99a | MIMAT0002412 | >hsa-miR-1250 | MIMAT0005902 | >dse-miR-305 | MIMAT0008790 |
| >ppy-miR-99a | MIMAT0002413 | >hsa-miR-1251 | MIMAT0005903 | >dse-miR-276a | MIMAT0008791 |
| >lla-miR-99a | MIMAT0002414 | >hsa-miR-1253 | MIMAT0005904 | >dse-miR-282 | MIMAT0008792 |
| >mne-miR-99a | MIMAT0002415 | >hsa-miR-1254 | MIMAT0005905 | >dse-miR-308 | MIMAT0008793 |
| >ppa-miR-99a | MIMAT0002416 | >hsa-miR-1255a | MIMAT0005906 | >dse-miR-277 | MIMAT0008794 |
| >ggo-miR-100 | MIMAT0002417 | >hsa-miR-1256 | MIMAT0005907 | >dse-miR-314 | MIMAT0008795 |
| >age-miR-100 | MIMAT0002418 | >hsa-miR-1257 | MIMAT0005908 | >dse-miR-100 | MIMAT0008796 |
| >ppa-miR-100 | MIMAT0002419 | >hsa-miR-1258 | MIMAT0005909 | >dse-miR-286 | MIMAT0008797 |
| >ppy-miR-100 | MIMAT0002420 | >hsa-miR-1259 | MIMAT0005910 | >dse-miR-284 | MIMAT0008798 |
| >ptr-miR-100 | MIMAT0002421 | >hsa-miR-1260 | MIMAT0005911 | >dse-miR-311 | MIMAT0008799 |
| >mml-miR-100 | MIMAT0002422 | >hsa-miR-548g | MIMAT0005912 | >dse-miR-9a | MIMAT0008800 |
| >sla-miR-100 | MIMAT0002423 | >hsa-miR-1261 | MIMAT0005913 | >dse-miR-304 | MIMAT0008801 |
| >lla-miR-100 | MIMAT0002424 | >hsa-miR-1262 | MIMAT0005914 | >dse-miR-33 | MIMAT0008802 |
| >ggo-miR-101 | MIMAT0002425 | >hsa-miR-1263 | MIMAT0005915 | >dse-miR-309 | MIMAT0008803 |
| >sla-miR-101 | MIMAT0002426 | >hsa-miR-548n | MIMAT0005916 | >dse-miR-306 | MIMAT0008804 |
| >age-miR-101 | MIMAT0002427 | >hsa-miR-548m | MIMAT0005917 | >dse-miR-288 | MIMAT0008806 |
| >ppa-miR-101 | MIMAT0002428 | >hsa-miR-1265 | MIMAT0005918 | >dse-miR-289 | MIMAT0008807 |
| >ppy-miR-101 | MIMAT0002429 | >hsa-miR-548o | MIMAT0005919 | >dse-miR-279 | MIMAT0008808 |
| >ptr-miR-101 | MIMAT0002430 | >hsa-miR-1266 | MIMAT0005920 | >dse-miR-285 | MIMAT0008809 |
| >mml-miR-101 | MIMAT0002431 | >hsa-miR-1267 | MIMAT0005921 | >dse-miR-312 | MIMAT0008810 |
| >lla-miR-101 | MIMAT0002432 | >hsa-miR-1268 | MIMAT0005922 | >dse-miR-307 | MIMAT0008811 |
| >mne-miR-101 | MIMAT0002433 | >hsa-miR-1269 | MIMAT0005923 | >dse-miR-9c | MIMAT0008812 |
| >ppy-miR-29b | MIMAT0002434 | >hsa-miR-1270 | MIMAT0005924 | >dse-miR-14 | MIMAT0008813 |
| >ptr-miR-29b | MIMAT0002435 | >hsa-miR-1272 | MIMAT0005925 | >dse-miR-1 | MIMAT0008814 |
| >ggo-miR-29b | MIMAT0002436 | >hsa-miR-1273 | MIMAT0005926 | >dse-miR-315 | MIMAT0008815 |
| >lla-miR-29b | MIMAT0002437 | >hsa-miR-1274a | MIMAT0005927 | >dse-miR-13b | MIMAT0008816 |
| >age-miR-29b | MIMAT0002438 | >hsa-miR-548h | MIMAT0005928 | >dse-miR-318 | MIMAT0008817 |
| >ppa-miR-29b | MIMAT0002439 | >hsa-miR-1275 | MIMAT0005929 | >dse-miR-316 | MIMAT0008818 |
| >sla-miR-29b | MIMAT0002440 | >hsa-miR-1276 | MIMAT0005930 | >dse-miR-2a | MIMAT0008819 |
| >mne-miR-29b | MIMAT0002441 | >hsa-miR-302e | MIMAT0005931 | >dse-miR-275 | MIMAT0008820 |
| >age-miR-103 | MIMAT0002442 | >hsa-miR-302f | MIMAT0005932 | >dse-miR-184 | MIMAT0008822 |
| >ggo-miR-103 | MIMAT0002443 | >hsa-miR-1277 | MIMAT0005933 | >dse-miR-31b | MIMAT0008823 |
| >ppa-miR-103 | MIMAT0002444 | >hsa-miR-548p | MIMAT0005934 | >dse-miR-219 | MIMAT0008824 |
| >ppy-miR-103 | MIMAT0002445 | >hsa-miR-548i | MIMAT0005935 | >dse-miR-31a | MIMAT0008825 |
| >ptr-miR-103 | MIMAT0002446 | >hsa-miR-1278 | MIMAT0005936 | >dse-miR-125 | MIMAT0008826 |
| >mml-miR-103 | MIMAT0002447 | >hsa-miR-1279 | MIMAT0005937 | >dse-miR-9b | MIMAT0008827 |
| >lla-miR-103 | MIMAT0002448 | >hsa-miR-1274b | MIMAT0005938 | >dse-miR-274 | MIMAT0008828 |
| >mne-miR-103 | MIMAT0002449 | >hsa-miR-1281 | MIMAT0005939 | >dse-miR-5 | MIMAT0008829 |
| >ppy-miR-105 | MIMAT0002450 | >hsa-miR-1282 | MIMAT0005940 | >dse-miR-263b | MIMAT0008830 |
| >ggo-miR-105 | MIMAT0002451 | >hsa-miR-1284 | MIMAT0005941 | >dse-miR-313 | MIMAT0008831 |
| >ppa-miR-105 | MIMAT0002452 | >hsa-miR-1288 | MIMAT0005942 | >dse-miR-iab-4-5p | MIMAT0008832 |
| >ptr-miR-105 | MIMAT0002453 | >hsa-miR-1292 | MIMAT0005943 | >dse-miR-iab-4-3p | MIMAT0008833 |
| >mml-miR-105 | MIMAT0002454 | >hsa-miR-1252 | MIMAT0005944 | >dse-miR-133 | MIMAT0008834 |
| >sla-miR-105 | MIMAT0002455 | >hsa-miR-1255b | MIMAT0005945 | >dse-miR-4 | MIMAT0008835 |
| >lla-miR-105 | MIMAT0002456 | >hsa-miR-1280 | MIMAT0005946 | >dse-miR-3 | MIMAT0008836 |
| >mne-miR-105 | MIMAT0002457 | >bol-miR824 | MIMAT0005597 | >dse-let-7 | MIMAT0008837 |
| >mml-miR-107 | MIMAT0002458 | >bra-miR824 | MIMAT0005598 | >dse-miR-283 | MIMAT0008838 |
| >ptr-miR-107 | MIMAT0002459 | >bna-miR824 | MIMAT0005599 | >dse-miR-124 | MIMAT0008839 |
| >ggo-miR-107 | MIMAT0002460 | >hsa-miR-1308 | MIMAT0005947 | >dsi-miR-34 | MIMAT0008840 |
| >ppy-miR-107 | MIMAT0002461 | >hsa-miR-664 | MIMAT0005949 | >dsi-miR-310 | MIMAT0008841 |
| >lla-miR-107 | MIMAT0002462 | >hsa-miR-1306 | MIMAT0005950 | >dsi-miR-4 | MIMAT0008842 |
| >mne-miR-107 | MIMAT0002463 | >hsa-miR-1307 | MIMAT0005951 | >dsi-miR-9a | MIMAT0008843 |
| >ppa-miR-107 | MIMAT0002464 | >bna-miR397a | MIMAT0005600 | >dsi-miR-87 | MIMAT0008844 |
| >ggo-miR-124a | MIMAT0002465 | >bna-miR397b | MIMAT0005601 | >dsi-miR-13b | MIMAT0008845 |
| >age-miR-124a | MIMAT0002466 | >bna-miR390a | MIMAT0005602 | >dsi-miR-125 | MIMAT0008846 |
| >ppa-miR-124a | MIMAT0002467 | >bna-miR390b | MIMAT0005603 | >dsi-miR-124 | MIMAT0008847 |
| >ppy-miR-124a | MIMAT0002468 | >bna-miR390c | MIMAT0005604 | >dsi-miR-14 | MIMAT0008848 |
| >ptr-miR-124a | MIMAT0002469 | >bna-miR171a | MIMAT0005605 | >dsi-miR-210 | MIMAT0008849 |
| >mml-miR-124a | MIMAT0002470 | >bna-miR171b | MIMAT0005606 | >dsi-miR-277 | MIMAT0008850 |
| >lla-miR-124a | MIMAT0002471 | >bna-miR171c | MIMAT0005607 | >dsi-miR-263a | MIMAT0008851 |
| >lla-miR-139 | MIMAT0002472 | >bna-miR171d | MIMAT0005608 | >dsi-miR-12 | MIMAT0008852 |
| >ppa-miR-139 | MIMAT0002473 | >bna-miR171e | MIMAT0005609 | >dsi-miR-2a | MIMAT0008853 |
| >ptr-miR-147a | MIMAT0002474 | >bna-miR171f | MIMAT0005610 | >dsi-miR-2c | MIMAT0008854 |
| >ppy-miR-147 | MIMAT0002475 | >bna-miR169a | MIMAT0005612 | >dsi-miR-iab-4-5p | MIMAT0008855 |
| >sla-miR-147 | MIMAT0002476 | >bna-miR169b | MIMAT0005613 | >dsi-miR-iab-4-3p | MIMAT0008856 |
| >mne-miR-147 | MIMAT0002477 | >bna-miR169c | MIMAT0005614 | >dsi-miR-311 | MIMAT0008857 |
| >ppa-miR-147 | MIMAT0002478 | >bna-miR169d | MIMAT0005615 | >dsi-miR-2b | MIMAT0008858 |
| >ggo-miR-7 | MIMAT0002479 | >bna-miR169e | MIMAT0005616 | >dsi-miR-286 | MIMAT0008859 |
| >ppy-miR-7 | MIMAT0002480 | >bna-miR169f | MIMAT0005617 | >dsi-miR-305 | MIMAT0008860 |
| >sla-miR-7 | MIMAT0002481 | >bna-miR169g | MIMAT0005618 | >dsi-bantam | MIMAT0008861 |
| >lla-miR-7 | MIMAT0002482 | >bna-miR169h | MIMAT0005619 | >dsi-miR-3 | MIMAT0008862 |
| >mne-miR-7 | MIMAT0002483 | >bna-miR169i | MIMAT0005620 | >dsi-miR-317 | MIMAT0008863 |
| >ppa-miR-7 | MIMAT0002484 | >bna-miR169j | MIMAT0005621 | >dsi-miR-314 | MIMAT0008864 |
| >ptr-miR-7 | MIMAT0002485 | >bna-miR169k | MIMAT0005622 | >dsi-miR-11 | MIMAT0008865 |
| >ggo-miR-10a | MIMAT0002486 | >bna-miR169l | MIMAT0005623 | >dsi-miR-33 | MIMAT0008866 |
| >ppy-miR-10a | MIMAT0002487 | >bna-miR169m | MIMAT0005624 | >dsi-miR-1 | MIMAT0008867 |
| >sla-miR-10a | MIMAT0002488 | >bna-miR168 | MIMAT0005625 | >dsi-miR-219 | MIMAT0008868 |
| >age-miR-10a | MIMAT0002489 | >bna-miR167a | MIMAT0005626 | >dsi-miR-316 | MIMAT0008869 |
| >ppa-miR-10a | MIMAT0002490 | >bna-miR167b | MIMAT0005627 | >dsi-miR-315 | MIMAT0008870 |
| >ggo-miR-10b | MIMAT0002491 | >bna-miR167c | MIMAT0005628 | >dsi-miR-263b | MIMAT0008871 |
| >mne-miR-10b | MIMAT0002492 | >bna-miR166a | MIMAT0005629 | >dsi-miR-100 | MIMAT0008872 |
| >ppa-miR-10b | MIMAT0002493 | >bna-miR166b | MIMAT0005630 | >dsi-miR-289 | MIMAT0008873 |
| >ggo-miR-34a | MIMAT0002494 | >bna-miR166c | MIMAT0005631 | >dsi-miR-313 | MIMAT0008874 |
| >age-miR-34a | MIMAT0002495 | >bna-miR166d | MIMAT0005632 | >dsi-miR-312 | MIMAT0008875 |
| >ppa-miR-34a | MIMAT0002496 | >bna-miR164 | MIMAT0005633 | >dsi-let-7 | MIMAT0008876 |
| >ppy-miR-34a | MIMAT0002497 | >bna-miR161 | MIMAT0005634 | >dsi-miR-308 | MIMAT0008877 |
| >ptr-miR-34a | MIMAT0002498 | >bna-miR159 | MIMAT0005635 | >dsi-miR-31b | MIMAT0008878 |
| >mml-miR-34a | MIMAT0002499 | >bna-miR156b | MIMAT0005636 | >dsi-miR-92b | MIMAT0008879 |
| >sla-miR-34a | MIMAT0002500 | >bna-miR1140a | MIMAT0005637 | >dsi-miR-275 | MIMAT0008880 |
| >lla-miR-34a | MIMAT0002501 | >bna-miR1140b | MIMAT0005638 | >dsi-miR-13a | MIMAT0008881 |
| >mne-miR-34a | MIMAT0002502 | >bna-miR156c | MIMAT0005639 | >dsi-miR-304 | MIMAT0008882 |
| >ggo-miR-181a | MIMAT0002503 | >vvi-miR156a | MIMAT0005640 | >dsi-miR-282 | MIMAT0008883 |
| >ppa-miR-181a | MIMAT0002504 | >vvi-miR156b | MIMAT0005641 | >dsi-miR-307 | MIMAT0008884 |
| >ptr-miR-181a | MIMAT0002505 | >vvi-miR156c | MIMAT0005642 | >dsi-miR-6 | MIMAT0008885 |
| >mml-miR-181a | MIMAT0002506 | >vvi-miR156d | MIMAT0005643 | >dsi-miR-31a | MIMAT0008886 |
| >sla-miR-181a | MIMAT0002507 | >vvi-miR156e | MIMAT0005644 | >dsi-miR-10 | MIMAT0008887 |
| >mne-miR-181a | MIMAT0002508 | >vvi-miR156f | MIMAT0005645 | >dsi-miR-285 | MIMAT0008888 |
| >mml-miR-181c | MIMAT0002509 | >vvi-miR156g | MIMAT0005646 | >dsi-miR-8 | MIMAT0008889 |
| >ptr-miR-181c | MIMAT0002510 | >vvi-miR156i | MIMAT0005647 | >dsi-miR-5 | MIMAT0008890 |
| >ggo-miR-181c | MIMAT0002511 | >vvi-miR159a | MIMAT0005648 | >dsi-miR-278 | MIMAT0008891 |
| >ppa-miR-181c | MIMAT0002512 | >vvi-miR159b | MIMAT0005649 | >dsi-miR-276a | MIMAT0008892 |
| >mml-miR-182 | MIMAT0002513 | >vvi-miR159c | MIMAT0005650 | >dsi-miR-283 | MIMAT0008893 |
| >ppy-miR-182 | MIMAT0002514 | >vvi-miR160a | MIMAT0005651 | >dsi-miR-309 | MIMAT0008894 |
| >ggo-miR-187 | MIMAT0002515 | >vvi-miR160b | MIMAT0005652 | >dsi-miR-276b | MIMAT0008895 |
| >ppy-miR-187 | MIMAT0002516 | >vvi-miR160c | MIMAT0005653 | >dsi-miR-279 | MIMAT0008896 |
| >mne-miR-187 | MIMAT0002517 | >vvi-miR160d | MIMAT0005654 | >dsi-miR-288 | MIMAT0008897 |
| >ppa-miR-187 | MIMAT0002518 | >vvi-miR160e | MIMAT0005655 | >dsi-miR-92a | MIMAT0008898 |
| >mml-miR-196a | MIMAT0002519 | >vvi-miR160f | MIMAT0005656 | >dsi-miR-287 | MIMAT0008899 |
| >ggo-miR-196 | MIMAT0002520 | >vvi-miR162 | MIMAT0005657 | >dsi-miR-133 | MIMAT0008900 |
| >ppy-miR-196 | MIMAT0002521 | >vvi-miR164a | MIMAT0005658 | >dsi-miR-7 | MIMAT0008901 |
| >ptr-miR-196a | MIMAT0002522 | >vvi-miR164b | MIMAT0005659 | >dsi-miR-284 | MIMAT0008902 |
| >ppy-miR-196-2 | MIMAT0002523 | >vvi-miR164c | MIMAT0005660 | >dsi-miR-184 | MIMAT0008904 |
| >lla-miR-196 | MIMAT0002524 | >vvi-miR164d | MIMAT0005661 | >dsi-miR-281 | MIMAT0008906 |
| >age-miR-196 | MIMAT0002525 | >vvi-miR166a | MIMAT0005662 | >dsi-miR-318 | MIMAT0008907 |
| >ppa-miR-196 | MIMAT0002526 | >vvi-miR166b | MIMAT0005663 | >dvi-miR-314 | MIMAT0008908 |
| >ggo-miR-199a | MIMAT0002527 | >vvi-miR166c | MIMAT0005664 | >dvi-miR-2a | MIMAT0008909 |
| >ppa-miR-199a | MIMAT0002528 | >vvi-miR166d | MIMAT0005665 | >dvi-miR-2c | MIMAT0008910 |
| >ppy-miR-199a | MIMAT0002529 | >vvi-miR166e | MIMAT0005666 | >dvi-miR-12 | MIMAT0008911 |
| >ptr-miR-199a-5p | MIMAT0002530 | >vvi-miR166f | MIMAT0005667 | >dvi-miR-13a | MIMAT0008912 |
| >mml-miR-199a | MIMAT0002531 | >vvi-miR166g | MIMAT0005668 | >dvi-miR-306 | MIMAT0008913 |
| >sla-miR-199a | MIMAT0002532 | >vvi-miR166h | MIMAT0005669 | >dvi-miR-279 | MIMAT0008915 |
| >lla-miR-199a | MIMAT0002533 | >vvi-miR167a | MIMAT0005670 | >dvi-miR-11 | MIMAT0008916 |
| >mne-miR-199a | MIMAT0002534 | >vvi-miR167b | MIMAT0005671 | >dvi-miR-125 | MIMAT0008917 |
| >ptr-miR-204 | MIMAT0002535 | >vvi-miR167c | MIMAT0005672 | >dvi-miR-313 | MIMAT0008918 |
| >ggo-miR-204 | MIMAT0002536 | >vvi-miR167d | MIMAT0005673 | >dvi-miR-263a | MIMAT0008919 |
| >ppy-miR-204 | MIMAT0002537 | >vvi-miR167e | MIMAT0005674 | >dvi-miR-274 | MIMAT0008920 |
| >sla-miR-204 | MIMAT0002538 | >vvi-miR168 | MIMAT0005675 | >dvi-miR-263b | MIMAT0008921 |
| >mne-miR-204 | MIMAT0002539 | >vvi-miR169a | MIMAT0005676 | >dvi-miR-318 | MIMAT0008922 |
| >ppa-miR-204 | MIMAT0002540 | >vvi-miR169y | MIMAT0005677 | >dvi-miR-6 | MIMAT0008923 |
| >ggo-miR-205 | MIMAT0002541 | >vvi-miR169c | MIMAT0005678 | >dvi-miR-9c | MIMAT0008924 |
| >age-miR-205 | MIMAT0002542 | >vvi-miR169d | MIMAT0005679 | >dvi-miR-9b | MIMAT0008925 |
| >ppa-miR-205 | MIMAT0002543 | >vvi-miR169e | MIMAT0005680 | >dvi-miR-281 | MIMAT0008927 |
| >ptr-miR-205 | MIMAT0002544 | >vvi-miR169f | MIMAT0005681 | >dvi-miR-210 | MIMAT0008928 |
| >lla-miR-205 | MIMAT0002545 | >vvi-miR169g | MIMAT0005682 | >dvi-miR-9a | MIMAT0008929 |
| >mne-miR-205 | MIMAT0002546 | >vvi-miR169j | MIMAT0005683 | >dvi-miR-287 | MIMAT0008930 |
| >mml-miR-211 | MIMAT0002547 | >vvi-miR169k | MIMAT0005684 | >dvi-miR-7 | MIMAT0008931 |
| >ppy-miR-211 | MIMAT0002548 | >vvi-miR169m | MIMAT0005685 | >dvi-miR-282 | MIMAT0008932 |
| >mne-miR-211 | MIMAT0002549 | >vvi-miR169p | MIMAT0005686 | >dvi-miR-315b | MIMAT0008933 |
| >ggo-miR-214 | MIMAT0002550 | >vvi-miR169r | MIMAT0005687 | >dvi-miR-310 | MIMAT0008934 |
| >age-miR-214 | MIMAT0002551 | >vvi-miR169s | MIMAT0005688 | >dvi-miR-283 | MIMAT0008935 |
| >ppa-miR-214 | MIMAT0002552 | >vvi-miR169t | MIMAT0005689 | >dvi-miR-133 | MIMAT0008936 |
| >ppy-miR-214 | MIMAT0002553 | >vvi-miR169u | MIMAT0005690 | >dvi-miR-278 | MIMAT0008937 |
| >ptr-miR-214 | MIMAT0002554 | >vvi-miR171a | MIMAT0005691 | >dvi-miR-13b | MIMAT0008938 |
| >mml-miR-214 | MIMAT0002555 | >vvi-miR171b | MIMAT0005692 | >dvi-miR-124 | MIMAT0008939 |
| >sla-miR-214 | MIMAT0002556 | >vvi-miR171c | MIMAT0005693 | >dvi-miR-305 | MIMAT0008940 |
| >mne-miR-214 | MIMAT0002557 | >vvi-miR171d | MIMAT0005694 | >dvi-miR-33 | MIMAT0008941 |
| >lca-miR-216 | MIMAT0002558 | >vvi-miR171e | MIMAT0005695 | >dvi-miR-276b | MIMAT0008942 |
| >ptr-miR-216a | MIMAT0002559 | >vvi-miR171f | MIMAT0005696 | >dvi-miR-276a | MIMAT0008943 |
| >ggo-miR-216 | MIMAT0002560 | >vvi-miR171h | MIMAT0005697 | >dvi-miR-315a | MIMAT0008944 |
| >ppy-miR-216 | MIMAT0002561 | >vvi-miR171i | MIMAT0005698 | >dvi-miR-316 | MIMAT0008945 |
| >ppa-miR-216 | MIMAT0002562 | >vvi-miR172a | MIMAT0005699 | >dvi-miR-277 | MIMAT0008946 |
| >ggo-miR-217 | MIMAT0002563 | >vvi-miR172b | MIMAT0005700 | >dvi-miR-317 | MIMAT0008947 |
| >ppa-miR-217 | MIMAT0002564 | >vvi-miR172c | MIMAT0005701 | >dvi-miR-184 | MIMAT0008949 |
| >ggo-miR-218 | MIMAT0002565 | >vvi-miR172d | MIMAT0005702 | >dvi-miR-286 | MIMAT0008950 |
| >age-miR-218 | MIMAT0002566 | >vvi-miR319b | MIMAT0005703 | >dvi-miR-2b | MIMAT0008951 |
| >ppa-miR-218 | MIMAT0002567 | >vvi-miR319c | MIMAT0005704 | >dvi-miR-288 | MIMAT0008952 |
| >lca-miR-218 | MIMAT0002568 | >vvi-miR319f | MIMAT0005705 | >dvi-miR-307 | MIMAT0008953 |
| >ppy-miR-218 | MIMAT0002569 | >vvi-miR319g | MIMAT0005706 | >dvi-miR-8 | MIMAT0008954 |
| >ptr-miR-218 | MIMAT0002570 | >vvi-miR390 | MIMAT0005707 | >dvi-miR-34 | MIMAT0008955 |
| >sla-miR-218 | MIMAT0002571 | >vvi-miR393b | MIMAT0005708 | >dvi-miR-4 | MIMAT0008956 |
| >lla-miR-218 | MIMAT0002572 | >vvi-miR394a | MIMAT0005709 | >dvi-miR-3 | MIMAT0008957 |
| >mne-miR-218 | MIMAT0002573 | >vvi-miR394b | MIMAT0005710 | >dvi-miR-309 | MIMAT0008958 |
| >mml-miR-218 | MIMAT0002574 | >vvi-miR395a | MIMAT0005711 | >dvi-miR-1 | MIMAT0008959 |
| >mml-miR-219 | MIMAT0002575 | >vvi-miR395b | MIMAT0005712 | >dvi-miR-10 | MIMAT0008960 |
| >ggo-miR-219 | MIMAT0002576 | >vvi-miR395c | MIMAT0005713 | >dvi-miR-5 | MIMAT0008961 |
| >ppy-miR-219 | MIMAT0002577 | >vvi-miR395d | MIMAT0005714 | >dvi-miR-92b | MIMAT0008962 |
| >mml-miR-220a | MIMAT0002578 | >vvi-miR395e | MIMAT0005715 | >dvi-miR-iab-4-5p | MIMAT0008963 |
| >ptr-miR-220a | MIMAT0002579 | >vvi-miR395f | MIMAT0005716 | >dvi-miR-iab-4-3p | MIMAT0008964 |
| >ggo-miR-220 | MIMAT0002580 | >vvi-miR395g | MIMAT0005717 | >dvi-miR-280 | MIMAT0008965 |
| >mne-miR-220 | MIMAT0002581 | >vvi-miR395h | MIMAT0005718 | >dvi-bantam | MIMAT0008966 |
| >ppa-miR-220 | MIMAT0002582 | >vvi-miR395i | MIMAT0005719 | >dvi-miR-31a | MIMAT0008967 |
| >mml-miR-221 | MIMAT0002583 | >vvi-miR395j | MIMAT0005720 | >dvi-miR-308 | MIMAT0008968 |
| >ggo-miR-221 | MIMAT0002584 | >vvi-miR395k | MIMAT0005721 | >dvi-miR-285 | MIMAT0008969 |
| >ppy-miR-221 | MIMAT0002585 | >vvi-miR395l | MIMAT0005722 | >dvi-miR-275 | MIMAT0008970 |
| >ppa-miR-221 | MIMAT0002586 | >vvi-miR395m | MIMAT0005723 | >dvi-miR-14 | MIMAT0008971 |
| >age-miR-222 | MIMAT0002587 | >vvi-miR396a | MIMAT0005724 | >dvi-miR-31b | MIMAT0008972 |
| >mml-miR-223 | MIMAT0002588 | >vvi-miR396b | MIMAT0005725 | >dvi-miR-100 | MIMAT0008973 |
| >ptr-miR-223 | MIMAT0002589 | >vvi-miR396d | MIMAT0005726 | >dvi-miR-87 | MIMAT0008974 |
| >ggo-miR-223 | MIMAT0002590 | >vvi-miR398a | MIMAT0005727 | >dvi-miR-79 | MIMAT0008975 |
| >ppy-miR-223 | MIMAT0002591 | >vvi-miR399a | MIMAT0005728 | >dvi-miR-304 | MIMAT0008976 |
| >sla-miR-223 | MIMAT0002592 | >vvi-miR399b | MIMAT0005729 | >dvi-miR-284 | MIMAT0008977 |
| >ppa-miR-223 | MIMAT0002593 | >vvi-miR399e | MIMAT0005730 | >dvi-miR-219 | MIMAT0008978 |
| >mml-miR-224 | MIMAT0002594 | >vvi-miR399g | MIMAT0005731 | >dvi-miR-92a | MIMAT0008979 |
| >ptr-miR-224 | MIMAT0002595 | >vvi-miR399h | MIMAT0005732 | >dvi-let-7 | MIMAT0008980 |
| >ggo-miR-224 | MIMAT0002596 | >vvi-miR408 | MIMAT0005733 | >dwi-miR-79 | MIMAT0008981 |
| >ppy-miR-224 | MIMAT0002597 | >vvi-miR479 | MIMAT0005734 | >dwi-miR-87 | MIMAT0008982 |
| >mne-miR-224 | MIMAT0002598 | >vvi-miR535a | MIMAT0005735 | >dwi-miR-34 | MIMAT0008983 |
| >ppa-miR-224 | MIMAT0002599 | >vvi-miR535b | MIMAT0005736 | >dwi-miR-285 | MIMAT0008984 |
| >ptr-miR-197 | MIMAT0002600 | >vvi-miR535c | MIMAT0005737 | >dwi-bantam | MIMAT0008985 |
| >ppy-miR-197 | MIMAT0002601 | >vvi-miR535d | MIMAT0005738 | >dwi-miR-306 | MIMAT0008986 |
| >mne-miR-197 | MIMAT0002602 | >vvi-miR535e | MIMAT0005739 | >dwi-miR-1 | MIMAT0008987 |
| >age-miR-197 | MIMAT0002603 | >age-miR-506 | MIMAT0005740 | >dwi-miR-184 | MIMAT0008989 |
| >ppa-miR-197 | MIMAT0002604 | >age-miR-507 | MIMAT0005741 | >dwi-miR-13b | MIMAT0008990 |
| >ggo-miR-198 | MIMAT0002605 | >age-miR-508 | MIMAT0005742 | >dwi-miR-92b | MIMAT0008991 |
| >age-miR-198 | MIMAT0002606 | >age-miR-509a | MIMAT0005743 | >dwi-miR-9c | MIMAT0008992 |
| >ppa-miR-198 | MIMAT0002607 | >age-miR-509b | MIMAT0005744 | >dwi-miR-284 | MIMAT0008993 |
| >ppy-miR-198 | MIMAT0002608 | >age-miR-510 | MIMAT0005745 | >dwi-miR-281 | MIMAT0008995 |
| >ptr-miR-198 | MIMAT0002609 | >age-miR-513b | MIMAT0005746 | >dwi-let-7 | MIMAT0008996 |
| >mml-miR-198 | MIMAT0002610 | >age-miR-513c | MIMAT0005747 | >dwi-miR-305 | MIMAT0008997 |
| >sla-miR-198 | MIMAT0002611 | >age-miR-513a | MIMAT0005748 | >dwi-miR-92a | MIMAT0008998 |
| >lla-miR-198 | MIMAT0002612 | >age-miR-513d | MIMAT0005749 | >dwi-miR-286 | MIMAT0008999 |
| >mne-miR-198 | MIMAT0002613 | >age-miR-513e | MIMAT0005750 | >dwi-miR-316 | MIMAT0009000 |
| >ptr-miR-30c | MIMAT0002614 | >age-miR-514 | MIMAT0005751 | >dwi-miR-279 | MIMAT0009001 |
| >lla-miR-30c | MIMAT0002615 | >ssy-miR-506 | MIMAT0005752 | >dwi-miR-263b | MIMAT0009002 |
| >mne-miR-30c | MIMAT0002616 | >ssy-miR-507 | MIMAT0005753 | >dwi-miR-287 | MIMAT0009003 |
| >ptr-miR-30d | MIMAT0002617 | >ssy-miR-508 | MIMAT0005754 | >dwi-miR-iab-4-5p | MIMAT0009004 |
| >ggo-miR-30d | MIMAT0002618 | >ssy-miR-509a | MIMAT0005755 | >dwi-miR-iab-4-3p | MIMAT0009005 |
| >mne-miR-30d | MIMAT0002619 | >ssy-miR-509b | MIMAT0005756 | >dwi-miR-2a | MIMAT0009006 |
| >ppa-miR-30d | MIMAT0002620 | >ssy-miR-510 | MIMAT0005757 | >dwi-miR-276a | MIMAT0009008 |
| >ppa-miR-1 | MIMAT0002621 | >ssy-miR-513c | MIMAT0005758 | >dwi-miR-7 | MIMAT0009009 |
| >mml-miR-181b | MIMAT0002623 | >ssy-miR-513b | MIMAT0005759 | >dwi-miR-11 | MIMAT0009010 |
| >ptr-miR-181b | MIMAT0002625 | >ssy-miR-513a | MIMAT0005760 | >dwi-miR-304 | MIMAT0009011 |
| >ppy-miR-181a | MIMAT0002626 | >ssy-miR-514 | MIMAT0005761 | >dwi-miR-219 | MIMAT0009012 |
| >ppy-miR-181b | MIMAT0002628 | >mml-miR-506 | MIMAT0005762 | >dwi-miR-8 | MIMAT0009013 |
| >ggo-miR-181b | MIMAT0002630 | >mml-miR-507 | MIMAT0005763 | >dwi-miR-283 | MIMAT0009014 |
| >lla-miR-181a | MIMAT0002631 | >mml-miR-508 | MIMAT0005764 | >dwi-miR-318 | MIMAT0009015 |
| >lla-miR-181b | MIMAT0002633 | >mml-miR-509 | MIMAT0005765 | >dwi-miR-2b | MIMAT0009016 |
| >mne-miR-181b | MIMAT0002635 | >mml-miR-510 | MIMAT0005766 | >dwi-miR-12 | MIMAT0009017 |
| >ppa-miR-181b | MIMAT0002637 | >mml-miR-513b | MIMAT0005767 | >dwi-miR-276b | MIMAT0009019 |
| >age-miR-15a | MIMAT0002638 | >mml-miR-513a | MIMAT0005768 | >dwi-miR-210 | MIMAT0009020 |
| >age-miR-16 | MIMAT0002639 | >mml-miR-514 | MIMAT0005769 | >dwi-miR-309 | MIMAT0009021 |
| >ggo-miR-15a | MIMAT0002640 | >ptr-miR-506 | MIMAT0005770 | >dwi-miR-6 | MIMAT0009022 |
| >ggo-miR-16 | MIMAT0002641 | >ptr-miR-507 | MIMAT0005771 | >dwi-miR-274 | MIMAT0009023 |
| >mne-miR-15a | MIMAT0002642 | >ptr-miR-508 | MIMAT0005772 | >dwi-miR-317 | MIMAT0009024 |
| >mne-miR-16 | MIMAT0002643 | >ptr-miR-509a | MIMAT0005773 | >dwi-miR-314 | MIMAT0009025 |
| >sla-miR-15a | MIMAT0002644 | >ptr-miR-509b | MIMAT0005774 | >dwi-miR-282 | MIMAT0009026 |
| >sla-miR-16 | MIMAT0002645 | >ptr-miR-510 | MIMAT0005775 | >dwi-miR-124 | MIMAT0009027 |
| >ppa-miR-15a | MIMAT0002646 | >ptr-miR-513a | MIMAT0005776 | >dwi-miR-280 | MIMAT0009028 |
| >ppa-miR-16 | MIMAT0002647 | >ptr-miR-513b | MIMAT0005777 | >dwi-miR-278 | MIMAT0009029 |
| >lca-miR-15a | MIMAT0002648 | >ptr-miR-514 | MIMAT0005778 | >dwi-miR-13a | MIMAT0009030 |
| >lca-miR-16 | MIMAT0002649 | >pbi-miR-506 | MIMAT0005779 | >dwi-miR-307 | MIMAT0009031 |
| >mml-miR-15a | MIMAT0002650 | >pbi-miR-507 | MIMAT0005780 | >dwi-miR-125 | MIMAT0009032 |
| >mml-miR-16 | MIMAT0002651 | >pbi-miR-508 | MIMAT0005781 | >dwi-miR-10 | MIMAT0009033 |
| >ppy-miR-15a | MIMAT0002652 | >pbi-miR-509 | MIMAT0005782 | >dwi-miR-9a | MIMAT0009034 |
| >ppy-miR-16 | MIMAT0002653 | >pbi-miR-510 | MIMAT0005783 | >dwi-miR-2c | MIMAT0009035 |
| >ptr-miR-15a | MIMAT0002654 | >pbi-miR-513a | MIMAT0005784 | >dwi-miR-315 | MIMAT0009036 |
| >ptr-miR-16 | MIMAT0002655 | >pbi-miR-513b | MIMAT0005785 | >dwi-miR-31b | MIMAT0009037 |
| >lla-miR-15a | MIMAT0002656 | >pbi-miR-513c | MIMAT0005786 | >dwi-miR-308 | MIMAT0009038 |
| >lla-miR-16 | MIMAT0002657 | >pbi-miR-514 | MIMAT0005787 | >dwi-miR-263a | MIMAT0009039 |
| >ggo-miR-17-5p | MIMAT0002658 | >hsa-miR-513b | MIMAT0005788 | >dwi-miR-277 | MIMAT0009040 |
| >ggo-miR-17-3p | MIMAT0002659 | >hsa-miR-513c | MIMAT0005789 | >dwi-miR-5 | MIMAT0009041 |
| >ggo-miR-18 | MIMAT0002660 | >hsa-miR-1321 | MIMAT0005952 | >dwi-miR-33 | MIMAT0009042 |
| >ggo-miR-19a | MIMAT0002661 | >hsa-miR-1322 | MIMAT0005953 | >dwi-miR-14 | MIMAT0009043 |
| >ggo-miR-20 | MIMAT0002662 | >hsa-miR-720 | MIMAT0005954 | >dwi-miR-31a | MIMAT0009044 |
| >ggo-miR-19b | MIMAT0002663 | >hsa-miR-1197 | MIMAT0005955 | >dwi-miR-275 | MIMAT0009045 |
| >ggo-miR-92 | MIMAT0002664 | >hsa-miR-1324 | MIMAT0005956 | >dwi-miR-3 | MIMAT0009046 |
| >lca-miR-17-5p | MIMAT0002665 | >oan-miR-9 | MIMAT0006792 | >dwi-miR-9b | MIMAT0009047 |
| >lca-miR-17-3p | MIMAT0002666 | >oan-miR-1325 | MIMAT0006794 | >dwi-miR-100 | MIMAT0009048 |
| >lca-miR-18 | MIMAT0002667 | >oan-miR-1326 | MIMAT0006795 | >dwi-miR-4 | MIMAT0009049 |
| >lca-miR-19a | MIMAT0002668 | >oan-miR-1327 | MIMAT0006796 | >dwi-miR-288 | MIMAT0009050 |
| >lca-miR-20 | MIMAT0002669 | >oan-miR-1328 | MIMAT0006797 | >dwi-miR-133 | MIMAT0009051 |
| >lca-miR-19b | MIMAT0002670 | >oan-miR-1329 | MIMAT0006798 | >dya-miR-274 | MIMAT0009052 |
| >lca-miR-92 | MIMAT0002671 | >oan-miR-1330 | MIMAT0006800 | >dya-miR-87 | MIMAT0009053 |
| >age-miR-17-5p | MIMAT0002672 | >oan-miR-129 | MIMAT0006801 | >dya-miR-307 | MIMAT0009054 |
| >age-miR-17-3p | MIMAT0002673 | >oan-miR-96 | MIMAT0006803 | >dya-miR-317 | MIMAT0009055 |
| >age-miR-18 | MIMAT0002674 | >oan-miR-183 | MIMAT0006805 | >dya-miR-13b | MIMAT0009056 |
| >age-miR-19a | MIMAT0002675 | >oan-miR-29a | MIMAT0006807 | >dya-bantam | MIMAT0009057 |
| >age-miR-20 | MIMAT0002676 | >oan-miR-29b | MIMAT0006809 | >dya-miR-4 | MIMAT0009058 |
| >age-miR-19b | MIMAT0002677 | >oan-miR-490 | MIMAT0006810 | >dya-miR-100 | MIMAT0009059 |
| >age-miR-92 | MIMAT0002678 | >oan-miR-140 | MIMAT0006813 | >dya-miR-281 | MIMAT0009061 |
| >ppa-miR-17-5p | MIMAT0002679 | >oan-miR-1331 | MIMAT0006814 | >dya-miR-279 | MIMAT0009062 |
| >ppa-miR-17-3p | MIMAT0002680 | >oan-miR-218 | MIMAT0006815 | >dya-miR-33 | MIMAT0009063 |
| >ppa-miR-18 | MIMAT0002681 | >oan-miR-33-5p | MIMAT0006817 | >dya-miR-283 | MIMAT0009064 |
| >ppa-miR-19a | MIMAT0002682 | >oan-miR-33a-3p | MIMAT0006818 | >dya-miR-280 | MIMAT0009065 |
| >ppa-miR-20 | MIMAT0002683 | >oan-miR-1332 | MIMAT0006819 | >dya-miR-8 | MIMAT0009066 |
| >ppa-miR-19b | MIMAT0002684 | >oan-miR-193 | MIMAT0006821 | >dya-miR-305 | MIMAT0009067 |
| >ppa-miR-92 | MIMAT0002685 | >oan-miR-365 | MIMAT0006823 | >dya-miR-306 | MIMAT0009068 |
| >ppy-miR-17-5p | MIMAT0002686 | >oan-miR-1333 | MIMAT0006824 | >dya-miR-9a | MIMAT0009070 |
| >ppy-miR-17-3p | MIMAT0002687 | >oan-miR-122 | MIMAT0006825 | >dya-miR-282 | MIMAT0009071 |
| >ppy-miR-18 | MIMAT0002688 | >oan-miR-1334 | MIMAT0006827 | >dya-miR-133 | MIMAT0009072 |
| >ppy-miR-19a | MIMAT0002689 | >oan-miR-1335 | MIMAT0006828 | >dya-miR-304 | MIMAT0009073 |
| >ppy-miR-20 | MIMAT0002690 | >oan-miR-1336 | MIMAT0006829 | >dya-miR-3 | MIMAT0009074 |
| >ppy-miR-19b | MIMAT0002691 | >oan-miR-875 | MIMAT0006830 | >dya-miR-2b | MIMAT0009075 |
| >ppy-miR-92 | MIMAT0002692 | >oan-miR-1337 | MIMAT0006832 | >dya-miR-14 | MIMAT0009076 |
| >ptr-miR-17-5p | MIMAT0002693 | >oan-miR-181a | MIMAT0006833 | >dya-miR-314 | MIMAT0009077 |
| >ptr-miR-17-3p | MIMAT0002694 | >oan-miR-181b | MIMAT0006835 | >dya-miR-6 | MIMAT0009078 |
| >ptr-miR-18a | MIMAT0002695 | >oan-miR-137a | MIMAT0006838 | >dya-miR-318 | MIMAT0009079 |
| >ptr-miR-19a | MIMAT0002696 | >oan-miR-1338 | MIMAT0006840 | >dya-miR-316 | MIMAT0009080 |
| >ptr-miR-20a | MIMAT0002697 | >oan-miR-204 | MIMAT0006841 | >dya-miR-34 | MIMAT0009081 |
| >ptr-miR-19b | MIMAT0002698 | >oan-miR-190a | MIMAT0006843 | >dya-miR-10 | MIMAT0009082 |
| >ptr-miR-92 | MIMAT0002699 | >oan-miR-15c | MIMAT0006845 | >dya-miR-1 | MIMAT0009083 |
| >mml-miR-17-5p | MIMAT0002700 | >oan-miR-16c | MIMAT0006847 | >dya-miR-315 | MIMAT0009084 |
| >mml-miR-17-3p | MIMAT0002701 | >oan-miR-106 | MIMAT0006849 | >dya-miR-309 | MIMAT0009085 |
| >mml-miR-18 | MIMAT0002702 | >oan-miR-18 | MIMAT0006851 | >dya-miR-278 | MIMAT0009086 |
| >mml-miR-19a | MIMAT0002703 | >oan-miR-20b | MIMAT0006853 | >dya-miR-284 | MIMAT0009087 |
| >mml-miR-20a | MIMAT0002704 | >oan-miR-19b | MIMAT0006856 | >dya-miR-285 | MIMAT0009088 |
| >mml-miR-19b | MIMAT0002705 | >oan-miR-92a | MIMAT0006858 | >dya-miR-iab-4-5p | MIMAT0009089 |
| >mml-miR-92a | MIMAT0002706 | >oan-miR-363 | MIMAT0006860 | >dya-miR-iab-4-3p | MIMAT0009090 |
| >sla-miR-17-5p | MIMAT0002707 | >oan-miR-1339 | MIMAT0006861 | >dya-miR-311a | MIMAT0009091 |
| >sla-miR-17-3p | MIMAT0002708 | >oan-miR-1a | MIMAT0006863 | >dya-miR-310 | MIMAT0009092 |
| >sla-miR-18 | MIMAT0002709 | >oan-miR-133a | MIMAT0006865 | >dya-miR-13a | MIMAT0009093 |
| >sla-miR-19a | MIMAT0002710 | >oan-miR-205 | MIMAT0006866 | >dya-miR-288 | MIMAT0009094 |
| >sla-miR-20 | MIMAT0002711 | >oan-miR-1340 | MIMAT0006869 | >dya-miR-210 | MIMAT0009095 |
| >sla-miR-19b | MIMAT0002712 | >oan-miR-135b | MIMAT0006870 | >dya-miR-125 | MIMAT0009096 |
| >sla-miR-92 | MIMAT0002713 | >oan-miR-1341 | MIMAT0006872 | >dya-miR-2c | MIMAT0009097 |
| >lla-miR-17-5p | MIMAT0002714 | >oan-miR-1342 | MIMAT0006873 | >dya-let-7 | MIMAT0009098 |
| >lla-miR-17-3p | MIMAT0002715 | >oan-miR-1343 | MIMAT0006874 | >dya-miR-277 | MIMAT0009099 |
| >lla-miR-18 | MIMAT0002716 | >oan-miR-196a | MIMAT0006876 | >dya-miR-308 | MIMAT0009100 |
| >lla-miR-19a | MIMAT0002717 | >oan-miR-137b | MIMAT0006878 | >dya-miR-5 | MIMAT0009101 |
| >lla-miR-20 | MIMAT0002718 | >oan-miR-1422a | MIMAT0006880 | >dya-miR-287 | MIMAT0009102 |
| >lla-miR-19b | MIMAT0002719 | >oan-miR-1422e | MIMAT0006881 | >dya-miR-31a | MIMAT0009103 |
| >lla-miR-92 | MIMAT0002720 | >oan-miR-1422h | MIMAT0006884 | >dya-miR-31b | MIMAT0009104 |
| >mne-miR-17-5p | MIMAT0002721 | >oan-miR-1422b | MIMAT0006886 | >dya-miR-124 | MIMAT0009105 |
| >mne-miR-17-3p | MIMAT0002722 | >oan-miR-1422f | MIMAT0006887 | >dya-miR-2a | MIMAT0009106 |
| >mne-miR-18 | MIMAT0002723 | >oan-miR-1422g | MIMAT0006889 | >dya-miR-219 | MIMAT0009107 |
| >mne-miR-19a | MIMAT0002724 | >oan-miR-1422c | MIMAT0006892 | >dya-miR-312 | MIMAT0009108 |
| >mne-miR-20 | MIMAT0002725 | >oan-miR-1422i | MIMAT0006894 | >dya-miR-286 | MIMAT0009109 |
| >mne-miR-19b | MIMAT0002726 | >oan-miR-1422k-5p | MIMAT0006895 | >dya-miR-263b | MIMAT0009110 |
| >mml-miR-194 | MIMAT0002727 | >oan-miR-1422k-3p | MIMAT0006896 | >dya-miR-92a | MIMAT0009111 |
| >mml-miR-215 | MIMAT0002728 | >oan-let-7b | MIMAT0006897 | >dya-miR-9c | MIMAT0009112 |
| >ptr-miR-194 | MIMAT0002729 | >oan-miR-219 | MIMAT0006900 | >dya-miR-9b | MIMAT0009113 |
| >ptr-miR-215 | MIMAT0002730 | >oan-miR-451 | MIMAT0006901 | >dya-miR-263a | MIMAT0009114 |
| >ppy-miR-194 | MIMAT0002731 | >oan-miR-144 | MIMAT0006903 | >dya-miR-275 | MIMAT0009115 |
| >ppy-miR-215 | MIMAT0002732 | >oan-miR-139 | MIMAT0006904 | >dya-miR-311b | MIMAT0009116 |
| >ggo-miR-194 | MIMAT0002733 | >oan-miR-1344 | MIMAT0006906 | >dya-miR-276a | MIMAT0009117 |
| >ggo-miR-215 | MIMAT0002734 | >oan-miR-130c | MIMAT0006908 | >dya-miR-289 | MIMAT0009118 |
| >mne-miR-194 | MIMAT0002735 | >oan-miR-301 | MIMAT0006910 | >dya-miR-7 | MIMAT0009119 |
| >mne-miR-215 | MIMAT0002736 | >oan-miR-30c | MIMAT0006911 | >dya-miR-92b | MIMAT0009120 |
| >age-miR-194 | MIMAT0002737 | >oan-miR-30a | MIMAT0006913 | >dya-miR-184 | MIMAT0009122 |
| >ggo-miR-23a | MIMAT0002738 | >oan-miR-460 | MIMAT0006915 | >dya-miR-11 | MIMAT0009123 |
| >ggo-miR-27a | MIMAT0002739 | >oan-miR-1345 | MIMAT0006917 | >dya-miR-79 | MIMAT0009124 |
| >ggo-miR-24 | MIMAT0002740 | >oan-miR-92b | MIMAT0006918 | >dya-miR-276b | MIMAT0009125 |
| >age-miR-23a | MIMAT0002741 | >oan-miR-1346 | MIMAT0006919 | >pta-miR1309 | MIMAT0009126 |
| >age-miR-27a | MIMAT0002742 | >oan-miR-208 | MIMAT0006921 | >pta-miR1310 | MIMAT0009127 |
| >ppa-miR-23a | MIMAT0002743 | >oan-miR-1420a | MIMAT0006923 | >pta-miR1311 | MIMAT0009128 |
| >ppa-miR-27a | MIMAT0002744 | >oan-miR-1420b | MIMAT0006925 | >pta-miR1312 | MIMAT0009129 |
| >lca-miR-23a | MIMAT0002745 | >oan-miR-1420c | MIMAT0006926 | >pta-miR1313 | MIMAT0009130 |
| >lca-miR-27a | MIMAT0002746 | >oan-miR-1420d | MIMAT0006928 | >pta-miR1314 | MIMAT0009131 |
| >ppy-miR-23a | MIMAT0002747 | >oan-miR-1420e | MIMAT0006929 | >pta-miR1315 | MIMAT0009132 |
| >ppy-miR-27a | MIMAT0002748 | >oan-miR-1347 | MIMAT0006930 | >pta-miR1316 | MIMAT0009133 |
| >ptr-miR-23a | MIMAT0002749 | >oan-miR-186 | MIMAT0006931 | >osa-miR1317 | MIMAT0009134 |
| >ptr-miR-27a | MIMAT0002750 | >oan-miR-1348 | MIMAT0006933 | >osa-miR1318 | MIMAT0009135 |
| >ptr-miR-24 | MIMAT0002751 | >oan-miR-1349 | MIMAT0006934 | >osa-miR1319 | MIMAT0009136 |
| >mml-miR-23a | MIMAT0002752 | >oan-miR-200a | MIMAT0006936 | >osa-miR1320 | MIMAT0009137 |
| >mml-miR-27a | MIMAT0002753 | >oan-miR-200b | MIMAT0006938 | >sly-miR156a | MIMAT0009138 |
| >sla-miR-23a | MIMAT0002754 | >oan-miR-1422l | MIMAT0006939 | >sly-miR156b | MIMAT0009139 |
| >sla-miR-27a | MIMAT0002755 | >oan-miR-1350 | MIMAT0006940 | >sly-miR156c | MIMAT0009140 |
| >mne-miR-23a | MIMAT0002756 | >oan-miR-103 | MIMAT0006942 | >sly-miR159 | MIMAT0009141 |
| >mne-miR-27a | MIMAT0002757 | >oan-miR-1351 | MIMAT0006943 | >sly-miR162 | MIMAT0009142 |
| >ggo-miR-106b | MIMAT0002758 | >oan-miR-1420f | MIMAT0006944 | >sly-miR172a | MIMAT0009143 |
| >ggo-miR-93 | MIMAT0002759 | >oan-miR-1352 | MIMAT0006945 | >sly-miR172b | MIMAT0009144 |
| >ggo-miR-25 | MIMAT0002760 | >oan-miR-1353 | MIMAT0006946 | >sly-miR319 | MIMAT0009145 |
| >age-miR-106b | MIMAT0002761 | >oan-miR-1354 | MIMAT0006947 | >sly-miR399 | MIMAT0009146 |
| >age-miR-93 | MIMAT0002762 | >oan-miR-499 | MIMAT0006949 | >jcv-miR-J1-5p | MIMAT0009147 |
| >ppa-miR-106b | MIMAT0002763 | >oan-miR-1355 | MIMAT0006951 | >jcv-miR-J1-3p | MIMAT0009148 |
| >ppa-miR-93 | MIMAT0002764 | >oan-miR-1356 | MIMAT0006953 | >bkv-miR-B1-5p | MIMAT0009149 |
| >ppa-miR-25 | MIMAT0002765 | >oan-miR-24 | MIMAT0006955 | >bkv-miR-B1-3p | MIMAT0009150 |
| >ppy-miR-106b | MIMAT0002766 | >oan-miR-27b | MIMAT0006957 | >bmo-miR-12 | MIMAT0009151 |
| >ppy-miR-93 | MIMAT0002767 | >oan-miR-23b | MIMAT0006959 | >bmo-miR-137 | MIMAT0009152 |
| >ppy-miR-25 | MIMAT0002768 | >oan-miR-1357 | MIMAT0006960 | >bmo-miR-190 | MIMAT0009153 |
| >ptr-miR-106b | MIMAT0002769 | >oan-miR-22 | MIMAT0006962 | >bmo-miR-285 | MIMAT0009154 |
| >ptr-miR-93 | MIMAT0002770 | >oan-miR-1421a | MIMAT0006963 | >bmo-miR-92 | MIMAT0009155 |
| >ptr-miR-25 | MIMAT0002771 | >oan-miR-1421b | MIMAT0006966 | >bmo-miR-925 | MIMAT0009156 |
| >mml-miR-106b | MIMAT0002772 | >oan-miR-1421c | MIMAT0006967 | >bmo-miR-927 | MIMAT0009157 |
| >mml-miR-93 | MIMAT0002773 | >oan-miR-1421d | MIMAT0006969 | >bmo-miR-929 | MIMAT0009158 |
| >mml-miR-25 | MIMAT0002774 | >oan-miR-1421e | MIMAT0006972 | >bmo-miR-930 | MIMAT0009159 |
| >sla-miR-106b | MIMAT0002775 | >oan-miR-1421f | MIMAT0006973 | >bmo-miR-932 | MIMAT0009160 |
| >sla-miR-93 | MIMAT0002776 | >oan-miR-1421g | MIMAT0006975 | >bmo-miR-9b | MIMAT0009161 |
| >lla-miR-106b | MIMAT0002777 | >oan-miR-1421h | MIMAT0006977 | >bmo-miR-iab-4-5p | MIMAT0009162 |
| >lla-miR-93 | MIMAT0002778 | >oan-miR-1421i | MIMAT0006979 | >bmo-miR-iab-4-3p | MIMAT0009163 |
| >lla-miR-25 | MIMAT0002779 | >oan-miR-1421j | MIMAT0006981 | >bmo-miR-278 | MIMAT0009164 |
| >mne-miR-106b | MIMAT0002780 | >oan-miR-142 | MIMAT0006982 | >osa-miR1846c-5p | MIMAT0009165 |
| >mne-miR-93 | MIMAT0002781 | >oan-let-7e | MIMAT0006984 | >osa-miR1846c-3p | MIMAT0009166 |
| >mne-miR-25 | MIMAT0002782 | >oan-miR-7 | MIMAT0006986 | | |

TABLE 2
miRNA* Sequences

| | | | | | |
|---|---|---|---|---|---|
| >cel-miR-56* | MIMAT0000027 | >dre-miR-181a* | MIMAT0001282 | >oan-miR-23b* | MIMAT0006958 |
| >hsa-let-7a* | MIMAT0004481 | >hsa-miR-424* | MIMAT0004749 | >oan-miR-22* | MIMAT0006961 |
| >hsa-let-7b* | MIMAT0004482 | >mmu-miR-425* | MIMAT0001342 | >oan-miR-1421a* | MIMAT0006964 |
| >hsa-let-7c* | MIMAT0004483 | >hsa-miR-425* | MIMAT0001343 | >oan-miR-1421b* | MIMAT0006965 |
| >hsa-let-7d* | MIMAT0004484 | >hsa-miR-18b* | MIMAT0004751 | >oan-miR-1421c* | MIMAT0006968 |
| >hsa-let-7e* | MIMAT0004485 | >hsa-miR-20b* | MIMAT0004752 | >oan-miR-1421d* | MIMAT0006970 |
| >hsa-let-7f-1* | MIMAT0004486 | >mmu-miR-431* | MIMAT0004753 | >oan-miR-1421e* | MIMAT0006971 |
| >hsa-let-7f-2* | MIMAT0004487 | >mmu-miR-433* | MIMAT0001419 | >oan-miR-1421f* | MIMAT0006974 |
| >hsa-miR-15a* | MIMAT0004488 | >hcmv-miR-UL22A* | MIMAT0001575 | >oan-miR-1421g-1* | MIMAT0006976 |
| >hsa-miR-16-1* | MIMAT0004489 | >hcmv-miR-UL36* | MIMAT0004754 | >oan-miR-1421h* | MIMAT0006978 |
| >hsa-miR-17* | MIMAT0000071 | >hcmv-miR-US25-1* | MIMAT0004755 | >oan-miR-1421i-1* | MIMAT0006980 |
| >hsa-miR-18a* | MIMAT0002891 | >hsa-miR-431* | MIMAT0004757 | >oan-miR-142* | MIMAT0006983 |
| >hsa-miR-19a* | MIMAT0004490 | >hsa-miR-452* | MIMAT0001636 | >oan-let-7e* | MIMAT0006985 |
| >hsa-miR-19b-1* | MIMAT0004491 | >dre-miR-9* | MIMAT0003156 | >oan-miR-7* | MIMAT0006987 |
| >hsa-miR-19b-2* | MIMAT0004492 | >dre-miR-10d* | MIMAT0003394 | >oan-miR-155* | MIMAT0006989 |
| >hsa-miR-20a* | MIMAT0004493 | >dre-miR-15a* | MIMAT0003395 | >oan-miR-26* | MIMAT0006992 |
| >hsa-miR-21* | MIMAT0004494 | >dre-miR-17a* | MIMAT0003396 | >oan-miR-206* | MIMAT0006994 |
| >hsa-miR-22* | MIMAT0004495 | >dre-miR-18b* | MIMAT0003397 | >oan-miR-133b* | MIMAT0006996 |
| >hsa-miR-23a* | MIMAT0004496 | >dre-miR-19a* | MIMAT0003398 | >oan-miR-215* | MIMAT0007001 |
| >hsa-miR-24-1* | MIMAT0000079 | >dre-miR-19b* | MIMAT0003399 | >oan-miR-153* | MIMAT0007004 |
| >hsa-miR-24-2* | MIMAT0004497 | >dre-miR-20a* | MIMAT0003400 | >oan-miR-146b* | MIMAT0007008 |
| >hsa-miR-25* | MIMAT0004498 | >dre-miR-27c* | MIMAT0003401 | >oan-miR-458* | MIMAT0007009 |
| >hsa-miR-26a-1* | MIMAT0004499 | >dre-miR-30e* | MIMAT0003402 | >oan-miR-449b* | MIMAT0007014 |
| >hsa-miR-26b* | MIMAT0004500 | >dre-miR-126* | MIMAT0003157 | >oan-miR-192* | MIMAT0007017 |
| >hsa-miR-27a* | MIMAT0004501 | >dre-miR-129* | MIMAT0003158 | >oan-miR-194* | MIMAT0007018 |
| >hsa-miR-29a* | MIMAT0004503 | >dre-miR-132* | MIMAT0003403 | >oan-miR-223* | MIMAT0007019 |
| >hsa-miR-30a* | MIMAT0000088 | >dre-miR-133a* | MIMAT0003404 | >oan-miR-1361* | MIMAT0007021 |
| >hsa-miR-31* | MIMAT0004504 | >dre-miR-133b* | MIMAT0003405 | >oan-miR-1362* | MIMAT0007024 |
| >hsa-miR-32* | MIMAT0004505 | >dre-miR-140* | MIMAT0003159 | >oan-miR-1363* | MIMAT0007026 |
| >hsa-miR-33a* | MIMAT0004506 | >dre-miR-202* | MIMAT0003406 | >oan-miR-1365* | MIMAT0007029 |
| >hsa-miR-92a-1* | MIMAT0004507 | >dre-miR-203b* | MIMAT0003407 | >oan-miR-1367* | MIMAT0007031 |
| >hsa-miR-92a-2* | MIMAT0004508 | >dre-miR-459* | MIMAT0003408 | >oan-let-7g* | MIMAT0007039 |
| >hsa-miR-93* | MIMAT0004509 | >mmu-miR-463* | MIMAT0002104 | >oan-miR-30d* | MIMAT0007042 |
| >hsa-miR-96* | MIMAT0004510 | >mmu-miR-467a* | MIMAT0002108 | >oan-miR-30b* | MIMAT0007044 |
| >hsa-miR-99a* | MIMAT0004511 | >mmu-miR-470* | MIMAT0004760 | >oan-miR-100* | MIMAT0007047 |
| >hsa-miR-100* | MIMAT0004512 | >ssc-miR-140* | MIMAT0006786 | >oan-miR-125* | MIMAT0007049 |
| >hsa-miR-101* | MIMAT0004513 | >kshv-miR-K12-9* | MIMAT0002184 | >oan-miR-15b* | MIMAT0007051 |
| >hsa-miR-29b-1* | MIMAT0004514 | >kshv-miR-K12-3* | MIMAT0002194 | >oan-miR-16b* | MIMAT0007053 |
| >hsa-miR-29b-2* | MIMAT0004515 | >mml-miR-181a* | MIMAT0002622 | >oan-miR-222b* | MIMAT0007056 |
| >hsa-miR-105* | MIMAT0004516 | >ptr-miR-181a* | MIMAT0002624 | >oan-miR-181c* | MIMAT0007059 |
| >hsa-miR-106a* | MIMAT0004517 | >ppy-miR-181a* | MIMAT0002627 | >oan-miR-23a* | MIMAT0007060 |
| >hsa-miR-16-2* | MIMAT0004518 | >ggo-miR-181a* | MIMAT0002629 | >oan-miR-27a* | MIMAT0007062 |
| >mmu-let-7g* | MIMAT0004519 | >lla-miR-181a* | MIMAT0002632 | >oan-miR-1375* | MIMAT0007065 |
| >mmu-let-7i* | MIMAT0004520 | >mne-miR-181a* | MIMAT0002634 | >oan-miR-1421k-1* | MIMAT0007067 |
| >mmu-miR-15b* | MIMAT0004521 | >ppa-miR-181a* | MIMAT0002636 | >oan-miR-1421l-1* | MIMAT0007069 |
| >mmu-miR-27b* | MIMAT0004522 | >hsa-miR-488* | MIMAT0002804 | >oan-miR-1419a* | MIMAT0007070 |
| >mmu-miR-29b* | MIMAT0004523 | >hsa-miR-202* | MIMAT0002810 | >oan-miR-1421m* | MIMAT0007073 |
| >mmu-miR-30a* | MIMAT0000129 | >hsa-miR-493* | MIMAT0002813 | >oan-miR-1421n-1* | MIMAT0007075 |
| >mmu-miR-30b* | MIMAT0004524 | >hsa-miR-432* | MIMAT0002815 | >oan-miR-1421o* | MIMAT0007077 |
| >mmu-miR-99b* | MIMAT0004525 | >hsa-miR-193b* | MIMAT0004767 | >oan-miR-1421p* | MIMAT0007079 |
| >mmu-miR-101a* | MIMAT0004526 | >hsa-miR-497* | MIMAT0004768 | >oan-miR-1421q* | MIMAT0007081 |
| >mmu-miR-124* | MIMAT0004527 | >hsa-miR-519e* | MIMAT0002828 | >oan-miR-1421r* | MIMAT0007083 |
| >mmu-miR-125b* | MIMAT0004529 | >hsa-miR-526b* | MIMAT0002836 | >oan-miR-1421s* | MIMAT0007085 |
| >mmu-miR-127* | MIMAT0004530 | >hsa-miR-523* | MIMAT0005449 | >oan-miR-1421t* | MIMAT0007087 |
| >mmu-miR-9* | MIMAT0000143 | >hsa-miR-518f* | MIMAT0002841 | >oan-miR-1421n-2* | MIMAT0009168 |
| >mmu-miR-133a* | MIMAT0003473 | >hsa-miR-518c* | MIMAT0002847 | >oan-miR-1421u* | MIMAT0007089 |
| >mmu-miR-135a* | MIMAT0004531 | >hsa-miR-517* | MIMAT0002851 | >oan-miR-150* | MIMAT0007091 |
| >mmu-miR-136* | MIMAT0004532 | >hsa-miR-516b* | MIMAT0002860 | >oan-miR-1422j* | MIMAT0007092 |
| >mmu-miR-140* | MIMAT0000152 | >hsa-miR-518e* | MIMAT0005450 | >oan-miR-1422d* | MIMAT0007095 |
| >mmu-miR-141* | MIMAT0004533 | >hsa-miR-522* | MIMAT0005451 | >oan-miR-590* | MIMAT0007096 |
| >mmu-miR-145* | MIMAT0004534 | >hsa-miR-519a* | MIMAT0005452 | >oan-miR-1422m* | MIMAT0007100 |
| >mmu-miR-150* | MIMAT0004535 | >hsa-miR-500* | MIMAT0002871 | >oan-miR-1422n* | MIMAT0007101 |
| >mmu-miR-154* | MIMAT0004537 | >hsa-miR-505* | MIMAT0004776 | >oan-miR-1422o* | MIMAT0007103 |
| >ath-miR172b* | MIMAT0000204 | >fru-miR-181a* | MIMAT0003005 | >oan-miR-1376* | MIMAT0007105 |
| >mmu-miR-10b* | MIMAT0004538 | >tni-miR-181a* | MIMAT0003006 | >oan-miR-34* | MIMAT0007109 |
| >mmu-miR-181a-2* | MIMAT0005443 | >mmu-miR-483* | MIMAT0003120 | >oan-miR-31* | MIMAT0007112 |
| >mmu-miR-183* | MIMAT0004539 | >rno-miR-1* | MIMAT0003162 | >oan-miR-124* | MIMAT0007113 |
| >mmu-miR-186* | MIMAT0004540 | >mmu-miR-485* | MIMAT0003129 | >oan-miR-1380* | MIMAT0007116 |
| >mmu-miR-24-1* | MIMAT0000218 | >ppt-miR319d.1* | MIMAT0004325 | >oan-miR-107* | MIMAT0007118 |
| >mmu-miR-191* | MIMAT0004542 | >ppt-miR319d.2* | MIMAT0004326 | >oan-miR-1381* | MIMAT0007120 |
| >hsa-miR-192* | MIMAT0004543 | >ppt-miR533a* | MIMAT0003137 | >oan-miR-10a* | MIMAT0007123 |
| >mmu-miR-193* | MIMAT0004544 | >hsa-miR-545* | MIMAT0004785 | >oan-miR-126* | MIMAT0007125 |
| >mmu-miR-200b* | MIMAT0004545 | >rno-miR-543* | MIMAT0003175 | >oan-miR-429* | MIMAT0007127 |
| >mmu-miR-203* | MIMAT0004547 | >mmu-miR-376c* | MIMAT0005295 | >oan-miR-551* | MIMAT0007129 |
| >hsa-miR-129* | MIMAT0004548 | >mmu-miR-20b* | MIMAT0004788 | >oan-miR-222a* | MIMAT0007132 |
| >hsa-miR-148a* | MIMAT0004549 | >mmu-miR-503* | MIMAT0004790 | >oan-miR-221* | MIMAT0007134 |
| >hsa-miR-30c-2* | MIMAT0004550 | >rno-miR-379* | MIMAT0004791 | >oan-miR-148* | MIMAT0007137 |
| >hsa-miR-30d* | MIMAT0004551 | >rno-miR-376a* | MIMAT0003197 | >oan-miR-196b* | MIMAT0007140 |
| >mmu-miR-30e* | MIMAT0000249 | >rno-miR-382* | MIMAT0003202 | >oan-miR-145* | MIMAT0007142 |
| >hsa-miR-7-1* | MIMAT0004553 | >rno-miR-363* | MIMAT0003209 | >oan-miR-143* | MIMAT0007143 |
| >hsa-miR-7-2* | MIMAT0004554 | >hsa-miR-92b* | MIMAT0004792 | >oan-miR-20a-1* | MIMAT0007146 |
| >hsa-miR-10a* | MIMAT0004555 | >hsa-miR-551b* | MIMAT0004794 | >oan-miR-19a* | MIMAT0007147 |
| >hsa-miR-10b* | MIMAT0004556 | >hsa-miR-589* | MIMAT0003256 | >oan-miR-20a-2* | MIMAT0009169 |
| >hsa-miR-34a* | MIMAT0004557 | >hsa-miR-550* | MIMAT0003257 | >oan-miR-92a-2* | MIMAT0009170 |
| >hsa-miR-181a-2* | MIMAT0004558 | >hsa-miR-593* | MIMAT0003261 | >oan-miR-191* | MIMAT0007153 |
| >hsa-miR-181c* | MIMAT0004559 | >hsa-miR-616* | MIMAT0003284 | >oan-miR-425* | MIMAT0007155 |
| >hsa-miR-182* | MIMAT0000260 | >hsa-miR-624* | MIMAT0003293 | >oan-miR-454* | MIMAT0007156 |
| >hsa-miR-183* | MIMAT0004560 | >hsa-miR-625* | MIMAT0004808 | >oan-miR-130b* | MIMAT0007158 |
| >hsa-miR-187* | MIMAT0004561 | >hsa-miR-629* | MIMAT0003298 | >oan-miR-21* | MIMAT0007161 |
| >hsa-miR-196a* | MIMAT0004562 | >hsa-miR-33b* | MIMAT0004811 | >oan-miR-15a* | MIMAT0007165 |
| >hsa-miR-181a* | MIMAT0000270 | >hsa-miR-411* | MIMAT0004813 | >oan-miR-16a* | MIMAT0007167 |
| >hsa-miR-214* | MIMAT0004564 | >gga-miR-9* | MIMAT0003350 | >oan-miR-1388* | MIMAT0007168 |
| >hsa-miR-218-1* | MIMAT0004565 | >gga-miR-146b* | MIMAT0003724 | >oan-miR-214* | MIMAT0007170 |
| >hsa-miR-218-2* | MIMAT0004566 | >gga-miR-202* | MIMAT0003354 | >oan-miR-199* | MIMAT0007172 |
| >hsa-miR-221* | MIMAT0004568 | >gga-miR-302b* | MIMAT0003356 | >oan-miR-128* | MIMAT0007175 |
| >hsa-miR-222* | MIMAT0004569 | >gga-miR-302c* | MIMAT0003358 | >oan-miR-1390* | MIMAT0007177 |
| >hsa-miR-223* | MIMAT0004570 | >mmu-miR-302b* | MIMAT0003373 | >oan-let-7f-2* | MIMAT0009171 |
| >cel-miR-253* | MIMAT0000309 | >mmu-miR-302c* | MIMAT0003375 | >oan-miR-802* | MIMAT0007182 |
| >hsa-miR-200b* | MIMAT0004571 | >rno-miR-378* | MIMAT0003378 | >oan-miR-99* | MIMAT0007184 |
| >dme-miR-184* | MIMAT0000330 | >ebv-miR-BART3* | MIMAT0003410 | >oan-miR-1391* | MIMAT0007185 |
| >dme-miR-276* | MIMAT0000336 | >ebv-miR-BART7* | MIMAT0004815 | >oan-miR-1392* | MIMAT0007188 |
| >dme-miR-281-1* | MIMAT0000344 | >ebv-miR-BART8* | MIMAT0003418 | >oan-miR-383* | MIMAT0007190 |
| >dme-miR-281-2* | MIMAT0000349 | >ebv-miR-BART9* | MIMAT0004816 | >oan-miR-135a* | MIMAT0007192 |
| >mmu-miR-293* | MIMAT0004573 | >ebv-miR-BART10* | MIMAT0004817 | >oan-miR-302* | MIMAT0007194 |
| >mmu-miR-294* | MIMAT0004574 | >ebv-miR-BART13* | MIMAT0004818 | >oan-miR-1397* | MIMAT0007200 |
| >mmu-miR-295* | MIMAT0004575 | >ebv-miR-BART14* | MIMAT0003425 | >oan-miR-30e* | MIMAT0007204 |
| >mmu-miR-299* | MIMAT0000377 | >hsa-miR-454* | MIMAT0003884 | >oan-miR-1400* | MIMAT0007205 |
| >mmu-miR-300* | MIMAT0004578 | >mmu-miR-744* | MIMAT0004820 | >oan-miR-1401* | MIMAT0007208 |
| >mmu-miR-302a* | MIMAT0004579 | >mmu-miR-374* | MIMAT0003728 | >oan-miR-1405* | MIMAT0007212 |
| >mmu-miR-34c* | MIMAT0004580 | >mmu-miR-674* | MIMAT0003741 | >oan-miR-1406* | MIMAT0007215 |
| >mmu-let-7d* | MIMAT0000384 | >mmu-miR-488* | MIMAT0003449 | >oan-miR-10b* | MIMAT0007219 |
| >mmu-miR-106b* | MIMAT0004582 | >mmu-miR-146b* | MIMAT0004826 | >oan-miR-138* | MIMAT0007220 |
| >mmu-miR-130b* | MIMAT0004583 | >mmu-miR-467b* | MIMAT0003478 | >oan-miR-217* | MIMAT0007222 |
| >dme-miR-306* | MIMAT0000394 | >mmu-miR-455* | MIMAT0003485 | >oan-miR-216* | MIMAT0007224 |
| >hsa-let-7g* | MIMAT0004584 | >mmu-miR-708* | MIMAT0003498 | >oan-miR-7-1* | MIMAT0009195 |
| >hsa-let-7i* | MIMAT0004585 | >mmu-miR-712* | MIMAT0003743 | >oan-miR-1409* | MIMAT0007227 |
| >hsa-miR-15b* | MIMAT0004586 | >ppt-miR1211* | MIMAT0003900 | >oan-miR-146a* | MIMAT0007231 |
| >hsa-miR-23b* | MIMAT0004587 | >ppt-miR390c* | MIMAT0004327 | >oan-let-7d* | MIMAT0007235 |
| >hsa-miR-27b* | MIMAT0004588 | >bta-miR-151* | MIMAT0003523 | >oan-miR-1421v* | MIMAT0007237 |
| >hsa-miR-30b* | MIMAT0004589 | >bta-miR-21* | MIMAT0003745 | >oan-miR-1421w* | MIMAT0007238 |
| >hsa-miR-122* | MIMAT0004590 | >bta-miR-126* | MIMAT0004328 | >oan-miR-1419b-1* | MIMAT0007241 |
| >hsa-miR-124* | MIMAT0004591 | >dre-miR-723* | MIMAT0003750 | >oan-miR-1421x* | MIMAT0007243 |
| >hsa-miR-125b-1* | MIMAT0004592 | >dre-miR-727* | MIMAT0003755 | >oan-miR-1421z* | MIMAT0007246 |
| >hsa-miR-130a* | MIMAT0004593 | >xtr-miR-9a* | MIMAT0003554 | >oan-miR-1419c* | MIMAT0007248 |
| >hsa-miR-132* | MIMAT0004594 | >xtr-miR-9b* | MIMAT0003556 | >oan-miR-1421aa* | MIMAT0007250 |
| >hsa-miR-135a* | MIMAT0004595 | >xtr-miR-126* | MIMAT0003587 | >oan-miR-1421ab* | MIMAT0007252 |
| >hsa-miR-138-2* | MIMAT0004596 | >xtr-miR-182* | MIMAT0003611 | >oan-miR-1419d* | MIMAT0007254 |
| >hsa-miR-141* | MIMAT0004598 | >xtr-miR-199a* | MIMAT0003619 | >oan-miR-1414* | MIMAT0007256 |
| >hsa-miR-143* | MIMAT0004599 | >xtr-miR-202* | MIMAT0003620 | >oan-miR-1421ac* | MIMAT0007258 |
| >hsa-miR-144* | MIMAT0004600 | >xtr-miR-181a-1* | MIMAT0003626 | >oan-miR-1421ad* | MIMAT0007260 |
| >hsa-miR-145* | MIMAT0004601 | >xtr-miR-181a-2* | MIMAT0005461 | >oan-miR-1419e* | MIMAT0007263 |
| >hsa-miR-191* | MIMAT0001618 | >xtr-miR-9* | MIMAT0003650 | >oan-miR-1421af* | MIMAT0007265 |
| >hsa-miR-9* | MIMAT0000442 | >xtr-miR-18a* | MIMAT0005462 | >oan-miR-187* | MIMAT0007266 |
| >hsa-miR-125b-2* | MIMAT0004603 | >xtr-miR-20a* | MIMAT0003670 | >oan-miR-32* | MIMAT0007269 |
| >hsa-miR-126* | MIMAT0000444 | >xtr-miR-22* | MIMAT0003671 | >oan-miR-873* | MIMAT0007273 |
| >hsa-miR-136* | MIMAT0004606 | >xtr-miR-29c* | MIMAT0003676 | >oan-miR-1417* | MIMAT0007275 |
| >hsa-miR-138-1* | MIMAT0004607 | >mmu-miR-676* | MIMAT0003781 | >oan-miR-1418* | MIMAT0007277 |
| >hsa-miR-146a* | MIMAT0004608 | >bta-miR-142* | MIMAT0003791 | >oan-miR-17* | MIMAT0007279 |
| >hsa-miR-149* | MIMAT0004609 | >bta-miR-193a* | MIMAT0003794 | >oan-let-7f-1* | MIMAT0007180 |
| >hsa-miR-150* | MIMAT0004610 | >bta-miR-545* | MIMAT0003806 | >gga-miR-1329* | MIMAT0007282 |
| >hsa-miR-154* | MIMAT0000453 | >bta-miR-455* | MIMAT0003836 | >gga-miR-1397* | MIMAT0007284 |
| >hsa-miR-185* | MIMAT0004611 | >mdv1-miR-M1* | MIMAT0005801 | >gga-miR-1416* | MIMAT0007286 |
| >hsa-miR-186* | MIMAT0004612 | >mdv1-miR-M2* | MIMAT0003922 | >gga-miR-22* | MIMAT0007287 |
| >hsa-miR-195* | MIMAT0004615 | >mdv1-miR-M4* | MIMAT0003925 | >gga-miR-551* | MIMAT0007289 |
| >mmu-miR-30c-1* | MIMAT0004616 | >mdv1-miR-M5* | MIMAT0005802 | >gga-miR-454* | MIMAT0007291 |
| >mmu-miR-30c-2* | MIMAT0005438 | >mdv1-miR-M7* | MIMAT0003928 | >gga-miR-33* | MIMAT0007293 |
| >mmu-miR-148a* | MIMAT0004617 | >sme-let-7b* | MIMAT0003954 | >mdv1-miR-M9* | MIMAT0005980 |
| >mmu-miR-196a* | MIMAT0004618 | >sme-miR-2a* | MIMAT0003964 | >oan-miR-1421y-2* | MIMAT0009172 |
| >mmu-miR-200a* | MIMAT0004619 | >sme-miR-2b* | MIMAT0003965 | >oan-miR-1421y-3* | MIMAT0009173 |
| >mmu-let-7a* | MIMAT0004620 | >sme-miR-10* | MIMAT0003974 | >oan-miR-1421ag* | MIMAT0007297 |
| >mmu-let-7b* | MIMAT0004621 | >sme-miR-36* | MIMAT0003981 | >oan-miR-1419b-2* | MIMAT0009174 |
| >mmu-let-7c-1* | MIMAT0004622 | >sme-miR-71b* | MIMAT0003986 | >oan-miR-1421ab-2* | MIMAT0007300 |
| >mmu-let-7c-2* | MIMAT0005439 | >sme-miR-124c* | MIMAT0003994 | >oan-miR-1421ac-2* | MIMAT0007302 |
| >mmu-let-7f* | MIMAT0004623 | >sme-miR-125a* | MIMAT0003997 | >oan-miR-1421ah* | MIMAT0007304 |
| >mmu-miR-15a* | MIMAT0004624 | >sme-miR-190a* | MIMAT0004002 | >oan-miR-1421ai* | MIMAT0007306 |
| >mmu-miR-16* | MIMAT0004625 | >sme-miR-190b* | MIMAT0004004 | >oan-miR-1421aj* | MIMAT0007308 |
| >mmu-miR-18a* | MIMAT0004626 | >sme-miR-277b* | MIMAT0004008 | >oan-miR-1422b-2* | MIMAT0009175 |
| >mmu-miR-20a* | MIMAT0004627 | >sme-miR-281* | MIMAT0004012 | >oan-miR-1421g-2* | MIMAT0009176 |
| >mmu-miR-21* | MIMAT0004628 | >mmu-miR-742* | MIMAT0004838 | >oan-miR-1421i-2* | MIMAT0009177 |
| >mmu-miR-22* | MIMAT0004629 | >mdo-miR-9* | MIMAT0004088 | >oan-miR-1421i-3* | MIMAT0009178 |
| >mmu-miR-24-2* | MIMAT0005440 | >mdo-miR-200a* | MIMAT0004157 | >oan-miR-1421k-2* | MIMAT0009179 |
| >mmu-miR-26b* | MIMAT0004630 | >ath-miR830* | MIMAT0004247 | >oan-miR-1421k-3* | MIMAT0009180 |
| >mmu-miR-29a* | MIMAT0004631 | >ath-miR844* | MIMAT0004267 | >oan-miR-1421l-2* | MIMAT0009181 |
| >mmu-miR-29c* | MIMAT0004632 | >bta-let-7a* | MIMAT0004330 | >oan-miR-1421ak* | MIMAT0007310 |
| >mmu-miR-27a* | MIMAT0004633 | >mmu-miR-879* | MIMAT0004843 | >oan-miR-1421al* | MIMAT0007312 |
| >mmu-miR-31* | MIMAT0004634 | >mmu-miR-881* | MIMAT0004845 | >oan-miR-1422p* | MIMAT0007314 |
| >mmu-miR-92a* | MIMAT0004635 | >mmu-miR-297a* | MIMAT0004864 | >oan-miR-1422q* | MIMAT0007317 |
| >mmu-miR-93* | MIMAT0004636 | >mmu-miR-297c* | MIMAT0004866 | >oan-miR-1420g* | MIMAT0007319 |
| >rno-miR-322* | MIMAT0000547 | >mmu-miR-467d* | MIMAT0004887 | >oan-miR-1419f* | MIMAT0007320 |
| >mmu-miR-322* | MIMAT0000549 | >hsa-miR-888* | MIMAT0004917 | >oan-miR-1419g* | MIMAT0007323 |
| >rno-miR-323* | MIMAT0004637 | >hsa-miR-541* | MIMAT0004919 | >gga-miR-1451* | MIMAT0007325 |
| >mmu-miR-325* | MIMAT0000558 | >hsa-miR-708* | MIMAT0004927 | >gga-miR-1456* | MIMAT0007334 |
| >rno-let-7d* | MIMAT0000563 | >mmu-miR-872* | MIMAT0004935 | >gga-miR-1467* | MIMAT0007346 |
| >rno-miR-330* | MIMAT0000568 | >mmu-miR-877* | MIMAT0004862 | >gga-miR-1551* | MIMAT0007406 |
| >mmu-miR-330* | MIMAT0000569 | >hsa-miR-744* | MIMAT0004946 | >gga-miR-1553* | MIMAT0007409 |
| >rno-miR-140* | MIMAT0000574 | >hsa-miR-877* | MIMAT0004950 | >gga-miR-1560* | MIMAT0007417 |
| >rno-miR-338* | MIMAT0004646 | >hsa-miR-374b* | MIMAT0004956 | >gga-miR-1562* | MIMAT0007421 |
| >rno-miR-129* | MIMAT0000601 | >ppt-miR898a* | MIMAT0004383 | >gga-miR-1564* | MIMAT0007424 |
| >rno-miR-20a* | MIMAT0000603 | >cre-miR905* | MIMAT0004964 | >gga-miR-1574* | MIMAT0007434 |
| >rno-miR-7a* | MIMAT0000607 | >cre-miR913* | MIMAT0004396 | >gga-miR-1591* | MIMAT0007453 |
| >rno-miR-151* | MIMAT0000614 | >rrv-miR-rR1-1* | MIMAT0004407 | >gga-miR-1595* | MIMAT0007459 |
| >hsa-miR-200c* | MIMAT0004657 | >rrv-miR-rR1-4* | MIMAT0004411 | >gga-miR-1596* | MIMAT0007461 |
| >osa-miR167a* | MIMAT0006780 | >rrv-miR-rR1-6* | MIMAT0004414 | >gga-miR-1597* | MIMAT0007462 |
| >hsa-miR-155* | MIMAT0004658 | >pta-miR946a* | MIMAT0006791 | >gga-miR-1614* | MIMAT0007481 |
| >mmu-miR-10a* | MIMAT0004659 | >cel-miR-1019* | MIMAT0005822 | >gga-miR-1621* | MIMAT0007489 |
| >mmu-miR-17* | MIMAT0000650 | >mdv2-miR-M15* | MIMAT0004453 | >gga-miR-1625* | MIMAT0007494 |
| >mmu-miR-19a* | MIMAT0004660 | >mdv2-miR-M17* | MIMAT0004456 | >gga-miR-1626* | MIMAT0007495 |
| >mmu-miR-28* | MIMAT0004661 | >mdv2-miR-M20* | MIMAT0004461 | >gga-miR-1627* | MIMAT0007497 |
| >mmu-miR-200c* | MIMAT0004663 | >mdv2-miR-M21* | MIMAT0004463 | >gga-miR-1632* | MIMAT0007505 |
| >mmu-miR-181a-1* | MIMAT0000660 | >mdv2-miR-M22* | MIMAT0004466 | >gga-miR-1643* | MIMAT0007518 |
| >mmu-miR-214* | MIMAT0004664 | >mdv2-miR-M28* | MIMAT0004475 | >gga-miR-1648* | MIMAT0007525 |
| >mmu-miR-218-1* | MIMAT0004665 | >rno-miR-872* | MIMAT0005283 | >gga-miR-1649* | MIMAT0007527 |
| >mmu-miR-218-2* | MIMAT0005444 | >mmu-miR-467e* | MIMAT0005294 | >gga-miR-1651* | MIMAT0007530 |
| >mmu-miR-33* | MIMAT0004666 | >rno-miR-708* | MIMAT0005332 | >gga-miR-1658* | MIMAT0007539 |
| >mmu-miR-199b* | MIMAT0000672 | >cre-miR1143* | MIMAT0005377 | >gga-miR-1663* | MIMAT0007544 |
| >mmu-miR-138* | MIMAT0004668 | >cre-miR1151a* | MIMAT0005394 | >gga-miR-1664* | MIMAT0007546 |
| >mmu-miR-7a* | MIMAT0004670 | >cre-miR1151b* | MIMAT0005396 | >gga-miR-1668* | MIMAT0007551 |
| >hsa-miR-194* | MIMAT0004671 | >cre-miR1153.2* | MIMAT0005399 | >gga-miR-1676* | MIMAT0007560 |
| >hsa-miR-106b* | MIMAT0004672 | >cre-miR1153.1* | MIMAT0005401 | >gga-miR-1677* | MIMAT0007562 |
| >hsa-miR-29c* | MIMAT0004673 | >cre-miR1154* | MIMAT0005403 | >gga-miR-1680* | MIMAT0007566 |
| >hsa-miR-30c-1* | MIMAT0004674 | >cre-miR1157* | MIMAT0005407 | >gga-miR-1684* | MIMAT0007571 |
| >hsa-miR-200a* | MIMAT0001620 | >cre-miR1162* | MIMAT0005415 | >gga-miR-1685* | MIMAT0007573 |
| >hsa-miR-302a* | MIMAT0000683 | >cre-miR1165* | MIMAT0005421 | >gga-miR-1687* | MIMAT0007577 |
| >hsa-miR-34b* | MIMAT0000685 | >cre-miR1169* | MIMAT0005426 | >gga-miR-1689* | MIMAT0007579 |
| >hsa-miR-99b* | MIMAT0004678 | >mcmv-miR-m01-2* | MIMAT0005535 | >gga-miR-1690* | MIMAT0007581 |
| >hsa-miR-130b* | MIMAT0004680 | >mcmv-miR-m01-3* | MIMAT0005537 | >gga-miR-449c* | MIMAT0007603 |
| >hsa-miR-30e* | MIMAT0000693 | >mcmv-miR-m01-4* | MIMAT0005539 | >gga-miR-1712* | MIMAT0007607 |
| >hsa-miR-26a-2* | MIMAT0004681 | >mcmv-miR-M23-2* | MIMAT0005544 | >gga-miR-1715* | MIMAT0007612 |
| >hsa-miR-363* | MIMAT0003385 | >mcmv-miR-m88-1* | MIMAT0005551 | >gga-miR-1720* | MIMAT0007617 |
| >hsa-miR-302b* | MIMAT0000714 | >mcmv-miR-m108-1* | MIMAT0005557 | >gga-miR-1722* | MIMAT0007621 |
| >hsa-miR-302c* | MIMAT0000716 | >mml-miR-1224* | MIMAT0005571 | >gga-miR-1729* | MIMAT0007630 |
| >hsa-miR-302d* | MIMAT0004685 | >hsa-miR-1226* | MIMAT0005576 | >gga-miR-1730* | MIMAT0007631 |
| >hsa-miR-367* | MIMAT0004686 | >hsa-miR-1228* | MIMAT0005582 | >gga-miR-1736* | MIMAT0007638 |
| >hsa-miR-373* | MIMAT0000725 | >hsa-miR-664* | MIMAT0005948 | >gga-miR-1740* | MIMAT0007644 |
| >hsa-miR-374a* | MIMAT0004688 | >oan-miR-9* | MIMAT0006793 | >gga-miR-1744* | MIMAT0007648 |
| >hsa-miR-376a* | MIMAT0003386 | >oan-miR-1329* | MIMAT0006799 | >gga-miR-1749* | MIMAT0007655 |
| >hsa-miR-377* | MIMAT0004689 | >oan-miR-129* | MIMAT0006802 | >gga-miR-1751* | MIMAT0007658 |
| >hsa-miR-378* | MIMAT0000731 | >oan-miR-96* | MIMAT0006804 | >gga-miR-1764* | MIMAT0007671 |
| >hsa-miR-379* | MIMAT0004690 | >oan-miR-29a-1* | MIMAT0006806 | >gga-miR-1769* | MIMAT0007677 |
| >hsa-miR-380* | MIMAT0000734 | >oan-miR-29b* | MIMAT0006808 | >gga-miR-1772* | MIMAT0007681 |
| >mmu-miR-376a* | MIMAT0003387 | >oan-miR-490* | MIMAT0006811 | >gga-miR-1773* | MIMAT0007683 |
| >mmu-miR-378* | MIMAT0000742 | >oan-miR-140* | MIMAT0006812 | >gga-miR-1781* | MIMAT0007692 |
| >mmu-miR-382* | MIMAT0004691 | >oan-miR-218* | MIMAT0006816 | >gga-miR-1784* | MIMAT0007696 |
| >hsa-miR-340* | MIMAT0000750 | >oan-miR-193* | MIMAT0006820 | >gga-miR-1791* | MIMAT0007704 |
| >hsa-miR-135b* | MIMAT0004698 | >oan-miR-365* | MIMAT0006822 | >gga-miR-1798* | MIMAT0007713 |
| >hsa-miR-148b* | MIMAT0004699 | >oan-miR-122* | MIMAT0006826 | >gga-miR-10a* | MIMAT0007732 |
| >hsa-miR-335* | MIMAT0004703 | >oan-miR-875* | MIMAT0006831 | >gga-miR-130c* | MIMAT0007733 |
| >rno-let-7b* | MIMAT0004705 | >oan-miR-181a* | MIMAT0006834 | >gga-miR-146c* | MIMAT0007736 |
| >rno-let-7e* | MIMAT0004706 | >oan-miR-181b* | MIMAT0006836 | >gga-miR-15c* | MIMAT0007738 |
| >rno-let-7i* | MIMAT0004707 | >oan-miR-137a* | MIMAT0006837 | >gga-miR-449b* | MIMAT0007744 |
| >rno-miR-9* | MIMAT0004708 | >oan-miR-1338* | MIMAT0006839 | >gga-miR-458* | MIMAT0007745 |
| >rno-miR-21* | MIMAT0004711 | >oan-miR-204* | MIMAT0006842 | >cfa-miR-219* | MIMAT0006729 |
| >rno-miR-22* | MIMAT0003152 | >oan-miR-190a* | MIMAT0006844 | >ssc-miR-199b* | MIMAT0007760 |
| >rno-miR-23a* | MIMAT0004712 | >oan-miR-15c* | MIMAT0006846 | >hsa-miR-1909* | MIMAT0007882 |
| >rno-miR-24-1* | MIMAT0003153 | >oan-miR-16c* | MIMAT0006848 | >hsa-miR-1911* | MIMAT0007886 |
| >rno-miR-24-2* | MIMAT0005441 | >oan-miR-106* | MIMAT0006850 | >hsa-miR-1914* | MIMAT0007890 |
| >rno-miR-25* | MIMAT0004713 | >oan-miR-18* | MIMAT0006852 | >hsa-miR-1915* | MIMAT0007891 |
| >rno-miR-26b* | MIMAT0004714 | >oan-miR-20b* | MIMAT0006854 | >bmo-miR-13a* | MIMAT0007896 |
| >rno-miR-27a* | MIMAT0004715 | >oan-miR-19b* | MIMAT0006855 | >bmo-miR-281* | MIMAT0007904 |
| >rno-miR-28* | MIMAT0004716 | >oan-miR-92a-1* | MIMAT0006857 | >tca-miR-276* | MIMAT0008380 |
| >rno-miR-29b-2* | MIMAT0004717 | >oan-miR-363* | MIMAT0006859 | >tca-miR-281* | MIMAT0008384 |
| >rno-miR-29a* | MIMAT0004718 | >oan-miR-1339* | MIMAT0006862 | >dan-miR-281-2* | MIMAT0009182 |
| >rno-miR-29b-1* | MIMAT0005445 | >oan-miR-133a* | MIMAT0006864 | >dan-miR-184* | MIMAT0008435 |
| >rno-miR-29c* | MIMAT0003154 | >oan-miR-29a-2* | MIMAT0009167 | >dan-miR-281-1* | MIMAT0008428 |
| >rno-miR-30c-1* | MIMAT0004719 | >oan-miR-205* | MIMAT0006867 | >der-miR-306* | MIMAT0008495 |
| >rno-miR-30e* | MIMAT0004720 | >oan-miR-1340* | MIMAT0006868 | >der-miR-281-2* | MIMAT0009183 |
| >rno-miR-30d* | MIMAT0004722 | >oan-miR-135b* | MIMAT0006871 | >der-miR-184* | MIMAT0008511 |
| >rno-miR-30a* | MIMAT0000809 | >oan-miR-1343* | MIMAT0006875 | >der-miR-281-1* | MIMAT0008501 |
| >rno-miR-30c-2* | MIMAT0005442 | >oan-miR-196a* | MIMAT0006877 | >dgr-miR-184* | MIMAT0008566 |
| >rno-miR-34c* | MIMAT0004723 | >oan-miR-1422a* | MIMAT0006879 | >dgr-miR-276b* | MIMAT0008573 |
| >rno-miR-99a* | MIMAT0004724 | >oan-miR-1422e* | MIMAT0006882 | >dgr-miR-276a* | MIMAT0008575 |
| >rno-miR-99b* | MIMAT0004725 | >oan-miR-1422h* | MIMAT0006883 | >dgr-miR-281-1* | MIMAT0008587 |
| >rno-miR-101a* | MIMAT0004726 | >oan-miR-1422b-1* | MIMAT0006885 | >dgr-miR-281-2* | MIMAT0009184 |
| >rno-miR-106b* | MIMAT0004727 | >oan-miR-1422f* | MIMAT0006888 | >dmo-miR-281-1* | MIMAT0008641 |
| >rno-miR-124* | MIMAT0004728 | >oan-miR-1422g* | MIMAT0006890 | >dmo-miR-281-2* | MIMAT0009185 |
| >rno-miR-125b* | MIMAT0004731 | >oan-miR-1422c* | MIMAT0006891 | >dmo-miR-306* | MIMAT0008649 |
| >rno-miR-126* | MIMAT0000831 | >oan-miR-1422i* | MIMAT0006893 | >dmo-miR-184* | MIMAT0008673 |
| >rno-miR-135a* | MIMAT0004732 | >oan-let-7b* | MIMAT0006898 | >dpe-miR-184* | MIMAT0008700 |
| >rno-miR-136* | MIMAT0004733 | >oan-miR-219* | MIMAT0006899 | >dpe-miR-281-1* | MIMAT0008727 |
| >rno-miR-138* | MIMAT0004734 | >oan-miR-144* | MIMAT0006902 | >dpe-miR-281-2* | MIMAT0009186 |
| >rno-miR-193* | MIMAT0004736 | >oan-miR-139* | MIMAT0006905 | >dse-miR-281-2* | MIMAT0009187 |
| >rno-miR-196a* | MIMAT0004737 | >oan-miR-130c* | MIMAT0006907 | >dse-miR-281-1* | MIMAT0008782 |
| >rno-miR-204* | MIMAT0004739 | >oan-miR-301* | MIMAT0006909 | >dse-miR-306* | MIMAT0008805 |
| >rno-miR-181a* | MIMAT0000884 | >oan-miR-30c* | MIMAT0006912 | >dse-miR-184* | MIMAT0008821 |
| >rno-miR-218* | MIMAT0004740 | >oan-miR-30a* | MIMAT0006914 | >dsi-miR-184* | MIMAT0008903 |
| >rno-miR-296* | MIMAT0000898 | >oan-miR-460* | MIMAT0006916 | >dsi-miR-281* | MIMAT0008905 |
| >ath-miR169g* | MIMAT0000912 | >oan-miR-208* | MIMAT0006920 | >dvi-miR-306* | MIMAT0008914 |
| >ebv-miR-BHRF1-2* | MIMAT0000996 | >oan-miR-1420a* | MIMAT0006922 | >dvi-miR-281-2* | MIMAT0009188 |
| >mmu-miR-376b* | MIMAT0003388 | >oan-miR-1420b* | MIMAT0006924 | >dvi-miR-281-1* | MIMAT0008926 |
| >mmu-miR-411* | MIMAT0001093 | >oan-miR-1420d* | MIMAT0006927 | >dvi-miR-184* | MIMAT0008948 |
| >gga-miR-99a* | MIMAT0006781 | >oan-miR-186* | MIMAT0006932 | >dwi-miR-184* | MIMAT0008988 |
| >gga-miR-181a* | MIMAT0001150 | >oan-miR-200a* | MIMAT0006935 | >dwi-miR-281-2* | MIMAT0009189 |
| >gga-miR-199* | MIMAT0003721 | >oan-miR-200b* | MIMAT0006937 | >dwi-miR-276a* | MIMAT0009007 |
| >gga-miR-140* | MIMAT0003722 | >oan-miR-103* | MIMAT0006941 | >dwi-miR-276b* | MIMAT0009018 |
| >gga-miR-126* | MIMAT0003723 | >oan-miR-1354* | MIMAT0006948 | >dwi-miR-281-1* | MIMAT0008994 |
| >dre-miR-10a* | MIMAT0003391 | >oan-miR-499* | MIMAT0006950 | >dya-miR-281-2* | MIMAT0009190 |
| >dre-miR-182* | MIMAT0001272 | >oan-miR-1355* | MIMAT0006952 | >dya-miR-306* | MIMAT0009069 |
| >dre-miR-199* | MIMAT0003155 | >oan-miR-24* | MIMAT0006954 | >dya-miR-281-1* | MIMAT0009060 |
| >dre-miR-210* | MIMAT0003392 | >oan-miR-27b* | MIMAT0006956 | >dya-miR-184* | MIMAT0009121 |

TABLE 3
Hairpin miRNA Precursors

| | | | | | |
|---|---|---|---|---|---|
| >cel-let-7 | MI0000001 | >lla-mir-17 | MI0003013 | >oan-mir-1421q | MI0006834 |
| >cel-lin-4 | MI0000002 | >lla-mir-18 | MI0003014 | >oan-mir-1421r | MI0006835 |
| >cel-mir-1 | MI0000003 | >lla-mir-19a | MI0003015 | >oan-mir-1421s | MI0006836 |
| >cel-mir-2 | MI0000004 | >lla-mir-20 | MI0003016 | >oan-mir-1421t | MI0006837 |
| >cel-mir-34 | MI0000005 | >lla-mir-19b-1 | MI0003017 | >oan-mir-1421n-2 | MI0006838 |
| >cel-mir-35 | MI0000006 | >lla-mir-92-1 | MI0003018 | >oan-mir-1421u | MI0006839 |
| >cel-mir-36 | MI0000007 | >mne-mir-17 | MI0003019 | >oan-mir-150 | MI0006840 |
| >cel-mir-37 | MI0000008 | >mne-mir-18 | MI0003020 | >oan-mir-1422j | MI0006841 |
| >cel-mir-38 | MI0000009 | >mne-mir-19a | MI0003021 | >oan-mir-1422d | MI0006842 |
| >cel-mir-39 | MI0000010 | >mne-mir-20 | MI0003022 | >oan-mir-590 | MI0006843 |
| >cel-mir-40 | MI0000011 | >mne-mir-19b-1 | MI0003023 | >oan-mir-130a | MI0006844 |
| >cel-mir-41 | MI0000012 | >mml-mir-194-1 | MI0003024 | >oan-mir-1422m | MI0006845 |
| >cel-mir-42 | MI0000013 | >mml-mir-215 | MI0003025 | >oan-mir-1422n | MI0006846 |
| >cel-mir-43 | MI0000014 | >ptr-mir-194 | MI0003026 | >oan-mir-1422o | MI0006847 |
| >cel-mir-44 | MI0000015 | >ptr-mir-215 | MI0003027 | >oan-mir-1376 | MI0006848 |
| >cel-mir-45 | MI0000016 | >ppy-mir-194 | MI0003028 | >oan-mir-1377 | MI0006849 |
| >cel-mir-46 | MI0000017 | >ppy-mir-215 | MI0003029 | >oan-mir-34 | MI0006850 |
| >cel-mir-47 | MI0000018 | >ggo-mir-194 | MI0003030 | >oan-mir-1378 | MI0006852 |
| >cel-mir-48 | MI0000019 | >ggo-mir-215 | MI0003031 | >oan-mir-31 | MI0006853 |
| >cel-mir-49 | MI0000020 | >mne-mir-194 | MI0003032 | >oan-mir-124 | MI0006854 |
| >cel-mir-50 | MI0000021 | >mne-mir-215 | MI0003033 | >oan-mir-1379 | MI0006855 |
| >cel-mir-51 | MI0000022 | >age-mir-194 | MI0003034 | >oan-mir-1380 | MI0006856 |
| >cel-mir-52 | MI0000023 | >ggo-mir-23a | MI0003035 | >oan-mir-107 | MI0006857 |
| >cel-mir-53 | MI0000024 | >ggo-mir-27a | MI0003036 | >oan-mir-1381 | MI0006858 |
| >cel-mir-54 | MI0000025 | >ggo-mir-24 | MI0003037 | >oan-mir-10a | MI0006859 |
| >cel-mir-55 | MI0000026 | >age-mir-23a | MI0003038 | >oan-mir-1382 | MI0006860 |
| >cel-mir-56 | MI0000027 | >age-mir-27a | MI0003039 | >oan-mir-126 | MI0006861 |
| >cel-mir-57 | MI0000028 | >ppa-mir-23a | MI0003040 | >oan-mir-429 | MI0006862 |
| >cel-mir-58 | MI0000029 | >ppa-mir-27a | MI0003041 | >oan-mir-551 | MI0006863 |
| >cel-mir-59 | MI0000030 | >ppa-mir-24-2 | MI0003042 | >oan-mir-1383 | MI0006864 |
| >cel-mir-60 | MI0000031 | >lca-mir-23a | MI0003043 | >oan-mir-222a | MI0006865 |
| >cel-mir-61 | MI0000032 | >lca-mir-27a | MI0003044 | >oan-mir-221 | MI0006866 |
| >cel-mir-62 | MI0000033 | >ppy-mir-23a | MI0003045 | >oan-mir-1384 | MI0006867 |
| >cel-mir-63 | MI0000034 | >ppy-mir-27a | MI0003046 | >oan-mir-148 | MI0006868 |
| >cel-mir-64 | MI0000035 | >ppy-mir-24-2 | MI0003047 | >oan-mir-196b | MI0006869 |
| >cel-mir-65 | MI0000036 | >ptr-mir-23a | MI0003048 | >oan-mir-145 | MI0006870 |
| >cel-mir-66 | MI0000037 | >ptr-mir-27a | MI0003049 | >oan-mir-143 | MI0006871 |
| >cel-mir-67 | MI0000038 | >ptr-mir-24-2 | MI0003050 | >oan-mir-20a-1 | MI0006872 |
| >cel-mir-70 | MI0000041 | >mml-mir-23a | MI0003051 | >oan-mir-19a | MI0006873 |
| >cel-mir-71 | MI0000042 | >mml-mir-27a | MI0003052 | >oan-mir-20a-2 | MI0006874 |
| >cel-mir-72 | MI0000043 | >mml-mir-24-2 | MI0003053 | >oan-mir-92a-2 | MI0006875 |
| >cel-mir-73 | MI0000044 | >sla-mir-23a | MI0003054 | >oan-mir-1b | MI0006876 |
| >cel-mir-74 | MI0000045 | >sla-mir-27a | MI0003055 | >oan-mir-133c | MI0006877 |
| >cel-mir-75 | MI0000046 | >mne-mir-23a | MI0003056 | >oan-mir-1385 | MI0006878 |
| >cel-mir-76 | MI0000047 | >mne-mir-27a | MI0003057 | >oan-mir-191 | MI0006879 |
| >cel-mir-77 | MI0000048 | >mne-mir-24-2 | MI0003058 | >oan-mir-425 | MI0006880 |
| >cel-mir-78 | MI0000049 | >ggo-mir-106b | MI0003059 | >oan-mir-454 | MI0006881 |
| >cel-mir-79 | MI0000050 | >ggo-mir-93 | MI0003060 | >oan-mir-130b | MI0006882 |
| >cel-mir-80 | MI0000051 | >ggo-mir-25 | MI0003061 | >oan-mir-21 | MI0006883 |
| >cel-mir-81 | MI0000052 | >age-mir-106b | MI0003062 | >oan-mir-1386 | MI0006884 |
| >cel-mir-82 | MI0000053 | >age-mir-93 | MI0003063 | >oan-mir-1387 | MI0006885 |
| >cel-mir-83 | MI0000054 | >ppa-mir-106b | MI0003064 | >oan-mir-15a | MI0006886 |
| >cel-mir-84 | MI0000055 | >ppa-mir-93 | MI0003065 | >oan-mir-16a | MI0006887 |
| >cel-mir-85 | MI0000056 | >ppa-mir-25 | MI0003066 | >oan-mir-1388 | MI0006888 |
| >cel-mir-86 | MI0000057 | >ppy-mir-106b | MI0003067 | >oan-mir-214 | MI0006889 |
| >cel-mir-87 | MI0000058 | >ppy-mir-93 | MI0003068 | >oan-mir-199 | MI0006890 |
| >cel-mir-90 | MI0000059 | >ppy-mir-25 | MI0003069 | >oan-mir-1389 | MI0006891 |
| >hsa-let-7a-1 | MI0000060 | >ptr-mir-106b | MI0003070 | >oan-mir-128-2 | MI0006892 |
| >hsa-let-7a-2 | MI0000061 | >ptr-mir-93 | MI0003071 | >oan-mir-1390 | MI0006893 |
| >hsa-let-7a-3 | MI0000062 | >ptr-mir-25 | MI0003072 | >oan-mir-98 | MI0006894 |
| >hsa-let-7b | MI0000063 | >mml-mir-106b | MI0003073 | >oan-let-7f-2 | MI0006895 |
| >hsa-let-7c | MI0000064 | >mml-mir-93 | MI0003074 | >oan-mir-802 | MI0006896 |
| >hsa-let-7d | MI0000065 | >mml-mir-25 | MI0003075 | >oan-mir-99 | MI0006897 |
| >hsa-let-7e | MI0000066 | >sla-mir-106b | MI0003076 | >oan-mir-1391 | MI0006898 |
| >hsa-let-7f-1 | MI0000067 | >sla-mir-93 | MI0003077 | >oan-mir-1392 | MI0006899 |
| >hsa-let-7f-2 | MI0000068 | >lla-mir-106b | MI0003078 | >oan-mir-383 | MI0006900 |
| >hsa-mir-15a | MI0000069 | >lla-mir-93 | MI0003079 | >oan-mir-1393 | MI0006901 |
| >hsa-mir-16-1 | MI0000070 | >lla-mir-25 | MI0003080 | >oan-mir-135a | MI0006902 |
| >hsa-mir-17 | MI0000071 | >mne-mir-106b | MI0003081 | >oan-mir-302-2 | MI0006903 |
| >hsa-mir-18a | MI0000072 | >mne-mir-93 | MI0003082 | >oan-mir-302-1 | MI0006904 |
| >hsa-mir-19a | MI0000073 | >mne-mir-25 | MI0003083 | >oan-mir-1394 | MI0006905 |
| >hsa-mir-19b-1 | MI0000074 | >mml-mir-183 | MI0003084 | >oan-mir-1395 | MI0006906 |
| >hsa-mir-19b-2 | MI0000075 | >mml-mir-96 | MI0003085 | >oan-mir-1396 | MI0006907 |
| >hsa-mir-20a | MI0000076 | >ptr-mir-183 | MI0003086 | >oan-mir-1397 | MI0006908 |
| >hsa-mir-21 | MI0000077 | >ptr-mir-96 | MI0003087 | >oan-mir-1398 | MI0006909 |
| >hsa-mir-22 | MI0000078 | >ggo-mir-183 | MI0003088 | >oan-mir-1399 | MI0006910 |
| >hsa-mir-23a | MI0000079 | >ggo-mir-96 | MI0003089 | >oan-mir-30e | MI0006911 |
| >hsa-mir-24-1 | MI0000080 | >sla-mir-183 | MI0003090 | >oan-mir-1400 | MI0006912 |
| >hsa-mir-24-2 | MI0000081 | >sla-mir-96 | MI0003091 | >oan-mir-1401 | MI0006913 |
| >hsa-mir-25 | MI0000082 | >mne-mir-183 | MI0003092 | >oan-mir-1402 | MI0006914 |
| >hsa-mir-26a-1 | MI0000083 | >mne-mir-96 | MI0003093 | >oan-mir-1403 | MI0006915 |
| >hsa-mir-26b | MI0000084 | >ppa-mir-183 | MI0003094 | >oan-mir-1404 | MI0006916 |
| >hsa-mir-27a | MI0000085 | >ppa-mir-96 | MI0003095 | >oan-mir-1405 | MI0006917 |
| >hsa-mir-28 | MI0000086 | >ggo-mir-106a | MI0003096 | >oan-mir-1406 | MI0006918 |
| >hsa-mir-29a | MI0000087 | >ggo-mir-19b-2 | MI0003097 | >oan-mir-138-1 | MI0006919 |
| >hsa-mir-30a | MI0000088 | >ggo-mir-92-2 | MI0003098 | >oan-mir-1407 | MI0006920 |
| >hsa-mir-31 | MI0000089 | >age-mir-106a | MI0003099 | >oan-mir-10b | MI0006921 |
| >hsa-mir-32 | MI0000090 | >age-mir-19b-2 | MI0003100 | >oan-mir-138-2 | MI0006922 |
| >hsa-mir-33a | MI0000091 | >age-mir-92-2 | MI0003101 | >oan-mir-217 | MI0006923 |
| >hsa-mir-92a-1 | MI0000093 | >ppa-mir-106a | MI0003102 | >oan-mir-216 | MI0006924 |
| >hsa-mir-92a-2 | MI0000094 | >ppa-mir-19b-2 | MI0003103 | >oan-mir-1408 | MI0006925 |
| >hsa-mir-93 | MI0000095 | >ppa-mir-92-2 | MI0003104 | >oan-mir-7-1 | MI0006926 |
| >hsa-mir-95 | MI0000097 | >lca-mir-19b-2 | MI0003105 | >oan-mir-1409 | MI0006927 |
| >hsa-mir-96 | MI0000098 | >lca-mir-92-2 | MI0003106 | >oan-mir-1410 | MI0006928 |
| >hsa-mir-98 | MI0000100 | >mml-mir-106a | MI0003107 | >oan-mir-1411 | MI0006929 |
| >hsa-mir-99a | MI0000101 | >mml-mir-19b-2 | MI0003108 | >oan-mir-103-1 | MI0006930 |
| >hsa-mir-100 | MI0000102 | >ppy-mir-106a | MI0003109 | >oan-mir-146a | MI0006931 |
| >hsa-mir-101-1 | MI0000103 | >ppy-mir-19b-2 | MI0003110 | >oan-mir-1412 | MI0006932 |
| >hsa-mir-29b-1 | MI0000105 | >ppy-mir-92-2 | MI0003111 | >oan-mir-1413 | MI0006933 |
| >hsa-mir-29b-2 | MI0000107 | >ptr-mir-106a | MI0003112 | >oan-let-7d | MI0006934 |
| >hsa-mir-103-2 | MI0000108 | >ptr-mir-19b-2 | MI0003113 | >oan-mir-1421v | MI0006935 |
| >hsa-mir-103-1 | MI0000109 | >ptr-mir-92-2 | MI0003114 | >oan-mir-1421w | MI0006936 |
| >hsa-mir-105-1 | MI0000111 | >sla-mir-106a | MI0003115 | >oan-mir-1419b-1 | MI0006937 |
| >hsa-mir-105-2 | MI0000112 | >sla-mir-19b-2 | MI0003116 | >oan-mir-1421x | MI0006938 |
| >hsa-mir-106a | MI0000113 | >sla-mir-92-2 | MI0003117 | >oan-mir-1421y-1 | MI0006939 |
| >hsa-mir-107 | MI0000114 | >lla-mir-19b-2 | MI0003118 | >oan-mir-1421z | MI0006940 |
| >hsa-mir-16-2 | MI0000115 | >lla-mir-92-2 | MI0003119 | >oan-mir-1419c-1 | MI0006941 |
| >dme-mir-1 | MI0000116 | >mne-mir-106a | MI0003120 | >oan-mir-1421aa | MI0006942 |
| >dme-mir-2a-1 | MI0000117 | >mne-mir-19b-2 | MI0003121 | >oan-mir-1421ab-1 | MI0006943 |
| >dme-mir-2a-2 | MI0000118 | >mne-mir-92 | MI0003122 | >oan-mir-1419d | MI0006944 |
| >dme-mir-2b-1 | MI0000119 | >hsa-mir-488 | MI0003123 | >oan-mir-1414 | MI0006945 |
| >dme-mir-2b-2 | MI0000120 | >hsa-mir-489 | MI0003124 | >oan-mir-1421ac-1 | MI0006946 |
| >dme-mir-3 | MI0000121 | >hsa-mir-490 | MI0003125 | >oan-mir-1421ad | MI0006947 |
| >dme-mir-4 | MI0000122 | >hsa-mir-491 | MI0003126 | >oan-mir-1421ae | MI0006948 |
| >dme-mir-5 | MI0000123 | >hsa-mir-511-1 | MI0003127 | >oan-mir-1419e | MI0006949 |
| >dme-mir-6-1 | MI0000124 | >hsa-mir-511-2 | MI0003128 | >oan-mir-1421af | MI0006950 |
| >dme-mir-6-2 | MI0000125 | >hsa-mir-146b | MI0003129 | >oan-mir-187 | MI0006951 |
| >dme-mir-6-3 | MI0000126 | >hsa-mir-202 | MI0003130 | >oan-mir-32 | MI0006952 |
| >dme-mir-7 | MI0000127 | >hsa-mir-492 | MI0003131 | >oan-mir-1415 | MI0006953 |
| >dme-mir-8 | MI0000128 | >hsa-mir-493 | MI0003132 | >oan-mir-1416 | MI0006954 |
| >dme-mir-9a | MI0000129 | >hsa-mir-432 | MI0003133 | >oan-mir-101-2 | MI0006955 |
| >dme-mir-10 | MI0000130 | >hsa-mir-494 | MI0003134 | >oan-mir-873 | MI0006956 |
| >dme-mir-11 | MI0000131 | >hsa-mir-495 | MI0003135 | >oan-mir-1417 | MI0006957 |
| >dme-mir-12 | MI0000132 | >hsa-mir-496 | MI0003136 | >oan-mir-1418 | MI0006959 |
| >dme-mir-13a | MI0000133 | >hsa-mir-193b | MI0003137 | >oan-mir-17 | MI0006960 |
| >dme-mir-13b-1 | MI0000134 | >hsa-mir-497 | MI0003138 | >oan-mir-7-3 | MI0006961 |
| >dme-mir-13b-2 | MI0000135 | >hsa-mir-181d | MI0003139 | >oan-let-7f-1 | MI0006962 |
| >dme-mir-14 | MI0000136 | >hsa-mir-512-1 | MI0003140 | >osa-MIR1423 | MI0006963 |
| >mmu-let-7g | MI0000137 | >hsa-mir-512-2 | MI0003141 | >osa-MIR1424 | MI0006964 |
| >mmu-let-7i | MI0000138 | >hsa-mir-498 | MI0003142 | >osa-MIR1425 | MI0006965 |
| >mmu-mir-1-1 | MI0000139 | >hsa-mir-520e | MI0003143 | >osa-MIR1426 | MI0006966 |
| >mmu-mir-15b | MI0000140 | >hsa-mir-515-1 | MI0003144 | >osa-MIR1427 | MI0006967 |
| >mmu-mir-23b | MI0000141 | >hsa-mir-519e | MI0003145 | >osa-MIR1428a | MI0006968 |
| >mmu-mir-27b | MI0000142 | >hsa-mir-520f | MI0003146 | >osa-MIR1429 | MI0006969 |
| >mmu-mir-29b-1 | MI0000143 | >hsa-mir-515-2 | MI0003147 | >osa-MIR1430 | MI0006970 |
| >mmu-mir-30a | MI0000144 | >hsa-mir-519c | MI0003148 | >osa-MIR1431 | MI0006971 |
| >mmu-mir-30b | MI0000145 | >hsa-mir-520a | MI0003149 | >osa-MIR1432 | MI0006972 |
| >mmu-mir-99a | MI0000146 | >hsa-mir-526b | MI0003150 | >osa-MIR1433 | MI0006973 |
| >mmu-mir-99b | MI0000147 | >hsa-mir-519b | MI0003151 | >osa-MIR444b | MI0006974 |
| >mmu-mir-101a | MI0000148 | >hsa-mir-525 | MI0003152 | >osa-MIR444c | MI0006975 |
| >mmu-mir-124-3 | MI0000150 | >hsa-mir-523 | MI0003153 | >osa-MIR444d | MI0006976 |
| >mmu-mir-125a | MI0000151 | >hsa-mir-518f | MI0003154 | >osa-MIR444e | MI0006977 |
| >mmu-mir-125b-2 | MI0000152 | >hsa-mir-520b | MI0003155 | >osa-MIR444f | MI0006978 |
| >mmu-mir-126 | MI0000153 | >hsa-mir-518b | MI0003156 | >gga-mir-1329 | MI0006979 |
| >mmu-mir-127 | MI0000154 | >hsa-mir-526a-1 | MI0003157 | >gga-mir-1397 | MI0006980 |
| >mmu-mir-128-1 | MI0000155 | >hsa-mir-520c | MI0003158 | >gga-mir-1416 | MI0006981 |
| >mmu-mir-130a | MI0000156 | >hsa-mir-518c | MI0003159 | >gga-mir-22 | MI0006982 |
| >mmu-mir-9-2 | MI0000157 | >hsa-mir-524 | MI0003160 | >gga-mir-551 | MI0006983 |
| >mmu-mir-132 | MI0000158 | >hsa-mir-517a | MI0003161 | >gga-mir-454 | MI0006984 |
| >mmu-mir-133a-1 | MI0000159 | >hsa-mir-519d | MI0003162 | >gga-mir-33-2 | MI0006985 |
| >mmu-mir-134 | MI0000160 | >hsa-mir-521-2 | MI0003163 | >osa-MIR810b | MI0006986 |
| >mmu-mir-135a-1 | MI0000161 | >hsa-mir-520d | MI0003164 | >mdv1-mir-M9 | MI0006987 |
| >mmu-mir-136 | MI0000162 | >hsa-mir-517b | MI0003165 | >mdv1-mir-M10 | MI0006988 |
| >mmu-mir-137 | MI0000163 | >hsa-mir-520g | MI0003166 | >mdv1-mir-M11 | MI0006989 |
| >mmu-mir-138-2 | MI0000164 | >hsa-mir-516b-2 | MI0003167 | >mdv1-mir-M12 | MI0006990 |
| >mmu-mir-140 | MI0000165 | >hsa-mir-526a-2 | MI0003168 | >mdv1-mir-M13 | MI0006991 |
| >mmu-mir-141 | MI0000166 | >hsa-mir-518e | MI0003169 | >gga-mir-1434 | MI0006992 |
| >mmu-mir-142 | MI0000167 | >hsa-mir-518a-1 | MI0003170 | >oan-mir-1421y-2 | MI0006993 |
| >mmu-mir-144 | MI0000168 | >hsa-mir-518d | MI0003171 | >oan-mir-1421y-3 | MI0006994 |
| >mmu-mir-145 | MI0000169 | >hsa-mir-516b-1 | MI0003172 | >oan-mir-1419c-2 | MI0006995 |
| >mmu-mir-146a | MI0000170 | >hsa-mir-518a-2 | MI0003173 | >oan-mir-1419c-3 | MI0006996 |
| >mmu-mir-149 | MI0000171 | >hsa-mir-517c | MI0003174 | >oan-mir-1421ag-1 | MI0006997 |
| >mmu-mir-150 | MI0000172 | >hsa-mir-520h | MI0003175 | >oan-mir-1419b-2 | MI0006998 |
| >mmu-mir-151 | MI0000173 | >hsa-mir-521-1 | MI0003176 | >oan-mir-1421ab-2 | MI0006999 |
| >mmu-mir-152 | MI0000174 | >hsa-mir-522 | MI0003177 | >oan-mir-1421ac-2 | MI0007000 |
| >mmu-mir-153 | MI0000175 | >hsa-mir-519a-1 | MI0003178 | >oan-mir-1421ah | MI0007001 |
| >mmu-mir-154 | MI0000176 | >hsa-mir-527 | MI0003179 | >oan-mir-1421ai | MI0007002 |
| >mmu-mir-155 | MI0000177 | >hsa-mir-516a-1 | MI0003180 | >oan-mir-1421aj | MI0007003 |
| >ath-MIR156a | MI0000178 | >hsa-mir-516a-2 | MI0003181 | >oan-mir-1422b-2 | MI0007004 |
| >ath-MIR156b | MI0000179 | >hsa-mir-519a-2 | MI0003182 | >oan-mir-1421g-2 | MI0007005 |
| >ath-MIR156c | MI0000180 | >hsa-mir-499 | MI0003183 | >oan-mir-1421g-3 | MI0007006 |
| >ath-MIR156d | MI0000181 | >hsa-mir-500 | MI0003184 | >oan-mir-1421i-2 | MI0007007 |
| >ath-MIR156e | MI0000182 | >hsa-mir-501 | MI0003185 | >oan-mir-1421i-3 | MI0007008 |
| >ath-MIR156f | MI0000183 | >hsa-mir-502 | MI0003186 | >oan-mir-1421k-2 | MI0007009 |
| >ath-MIR157a | MI0000184 | >hsa-mir-450a-2 | MI0003187 | >oan-mir-1421k-3 | MI0007010 |
| >ath-MIR157b | MI0000185 | >hsa-mir-503 | MI0003188 | >oan-mir-1421l-2 | MI0007011 |
| >ath-MIR157c | MI0000186 | >hsa-mir-504 | MI0003189 | >oan-mir-1421p-2 | MI0007012 |
| >ath-MIR157d | MI0000187 | >hsa-mir-505 | MI0003190 | >oan-mir-1421ak | MI0007013 |
| >ath-MIR158a | MI0000188 | >hsa-mir-513a-1 | MI0003191 | >oan-mir-1421al | MI0007014 |
| >ath-MIR159a | MI0000189 | >hsa-mir-513a-2 | MI0003192 | >oan-mir-1421am | MI0007015 |
| >ath-MIR160a | MI0000190 | >hsa-mir-506 | MI0003193 | >oan-mir-1422p | MI0007016 |
| >ath-MIR160b | MI0000191 | >hsa-mir-507 | MI0003194 | >oan-mir-1422q | MI0007017 |
| >ath-MIR160c | MI0000192 | >hsa-mir-508 | MI0003195 | >oan-mir-1420g | MI0007018 |
| >ath-MIR161 | MI0000193 | >hsa-mir-509-1 | MI0003196 | >oan-mir-1419f | MI0007019 |
| >ath-MIR162a | MI0000194 | >hsa-mir-510 | MI0003197 | >oan-mir-1419g | MI0007020 |
| >ath-MIR162b | MI0000195 | >hsa-mir-514-1 | MI0003198 | >osa-MIR1435 | MI0007021 |
| >ath-MIR163 | MI0000196 | >hsa-mir-514-2 | MI0003199 | >osa-MIR1436 | MI0007022 |
| >ath-MIR164a | MI0000197 | >hsa-mir-514-3 | MI0003200 | >osa-MIR1437 | MI0007024 |
| >ath-MIR164b | MI0000198 | >osa-MIR528 | MI0003201 | >osa-MIR1438 | MI0007025 |
| >ath-MIR165a | MI0000199 | >osa-MIR529a | MI0003202 | >osa-MIR1440 | MI0007028 |
| >ath-MIR165b | MI0000200 | >osa-MIR530 | MI0003203 | >osa-MIR1441 | MI0007029 |
| >ath-MIR166a | MI0000201 | >osa-MIR531 | MI0003204 | >osa-MIR1442 | MI0007030 |
| >ath-MIR166b | MI0000202 | >hsa-mir-532 | MI0003205 | >osa-MIR1439 | MI0007031 |
| >ath-MIR166c | MI0000203 | >mmu-mir-532 | MI0003206 | >ptc-MIR171l | MI0007032 |
| >ath-MIR166d | MI0000204 | >fru-let-7h | MI0003207 | >ptc-MIR171m | MI0007033 |
| >ath-MIR166e | MI0000205 | >tni-let-7h | MI0003208 | >ptc-MIR171n | MI0007034 |
| >ath-MIR166f | MI0000206 | >fru-mir-223 | MI0003209 | >ptc-MIR530a | MI0007035 |
| >ath-MIR166g | MI0000207 | >tni-mir-223 | MI0003210 | >ptc-MIR530b | MI0007036 |
| >ath-MIR167a | MI0000208 | >fru-mir-124-3 | MI0003211 | >ptc-MIR1444a | MI0007037 |
| >ath-MIR167b | MI0000209 | >tni-mir-124-3 | MI0003212 | >ptc-MIR1444b | MI0007038 |
| >ath-MIR168a | MI0000210 | >fru-let-7e | MI0003213 | >ptc-MIR1444c | MI0007039 |
| >ath-MIR168b | MI0000211 | >tni-let-7e | MI0003214 | >ptc-MIR1445 | MI0007040 |
| >ath-MIR169a | MI0000212 | >fru-mir-219-2 | MI0003215 | >ptc-MIR827 | MI0007041 |
| >ath-MIR170 | MI0000213 | >tni-mir-219-2 | MI0003216 | >ptc-MIR1446a | MI0007042 |
| >ath-MIR171a | MI0000214 | >fru-mir-101a | MI0003217 | >ptc-MIR1446b | MI0007043 |
| >ath-MIR172a | MI0000215 | >tni-mir-101a | MI0003218 | >ptc-MIR1446c | MI0007044 |
| >ath-MIR172b | MI0000216 | >fru-mir-222 | MI0003219 | >ptc-MIR1446d | MI0007045 |
| >ath-MIR173 | MI0000217 | >tni-mir-222 | MI0003220 | >ptc-MIR1446e | MI0007046 |
| >ath-MIR159b | MI0000218 | >fru-mir-221 | MI0003221 | >ptc-MIR1447 | MI0007047 |
| >mmu-mir-10b | MI0000221 | >tni-mir-221 | MI0003222 | >ptc-MIR1448 | MI0007048 |
| >mmu-mir-129-1 | MI0000222 | >fru-mir-142a | MI0003223 | >ptc-MIR1449 | MI0007049 |
| >mmu-mir-181a-2 | MI0000223 | >tni-mir-142a | MI0003224 | >ptc-MIR1450 | MI0007050 |
| >mmu-mir-182 | MI0000224 | >fru-mir-9-1 | MI0003225 | >osa-MIR827 | MI0007051 |
| >mmu-mir-183 | MI0000225 | >tni-mir-9-1 | MI0003226 | >pta-MIR482c | MI0007052 |
| >mmu-mir-184 | MI0000226 | >fru-mir-23a-2 | MI0003227 | >pta-MIR482d | MI0007053 |
| >mmu-mir-185 | MI0000227 | >tni-mir-23a-2 | MI0003228 | >gga-mir-1451 | MI0007054 |
| >mmu-mir-186 | MI0000228 | >fru-mir-25 | MI0003229 | >gga-mir-460b | MI0007055 |
| >mmu-mir-187 | MI0000229 | >tni-mir-25 | MI0003230 | >gga-mir-1452 | MI0007056 |
| >mmu-mir-188 | MI0000230 | >fru-mir-17-1 | MI0003231 | >gga-mir-1306 | MI0007057 |
| >mmu-mir-24-1 | MI0000231 | >tni-mir-17-1 | MI0003232 | >gga-mir-1453 | MI0007058 |
| >mmu-mir-190 | MI0000232 | >fru-mir-18 | MI0003233 | >gga-mir-1454 | MI0007059 |
| >mmu-mir-191 | MI0000233 | >tni-mir-18 | MI0003234 | >gga-mir-1455 | MI0007060 |
| >hsa-mir-192 | MI0000234 | >fru-mir-19a | MI0003235 | >gga-mir-1456 | MI0007061 |
| >mmu-mir-193 | MI0000235 | >tni-mir-19a | MI0003236 | >gga-mir-1457 | MI0007062 |
| >mmu-mir-194-1 | MI0000236 | >fru-mir-19b | MI0003237 | >gga-mir-1458 | MI0007063 |
| >mmu-mir-195 | MI0000237 | >tni-mir-19b | MI0003238 | >gga-mir-1459 | MI0007064 |
| >hsa-mir-196a-1 | MI0000238 | >fru-mir-92-2 | MI0003239 | >gga-mir-1460 | MI0007065 |
| >hsa-mir-197 | MI0000239 | >tni-mir-92-2 | MI0003240 | >gga-mir-1461 | MI0007066 |
| >hsa-mir-198 | MI0000240 | >fru-mir-184 | MI0003241 | >gga-mir-1462 | MI0007067 |
| >mmu-mir-199a-1 | MI0000241 | >tni-mir-184 | MI0003242 | >gga-mir-1463 | MI0007068 |
| >hsa-mir-199a-1 | MI0000242 | >dre-mir-184-2 | MI0003243 | >gga-mir-1464 | MI0007069 |
| >mmu-mir-200b | MI0000243 | >fru-let-7a-3 | MI0003244 | >gga-mir-1465 | MI0007070 |
| >mmu-mir-201 | MI0000244 | >tni-let-7a-3 | MI0003245 | >gga-mir-1466 | MI0007071 |
| >mmu-mir-202 | MI0000245 | >fru-mir-187 | MI0003246 | >gga-mir-1467 | MI0007072 |
| >mmu-mir-203 | MI0000246 | >tni-mir-187 | MI0003247 | >hiv1-mir-TAR | MI0007073 |
| >mmu-mir-204 | MI0000247 | >fru-mir-137 | MI0003248 | >hsa-mir-1469 | MI0007074 |
| >mmu-mir-205 | MI0000248 | >tni-mir-137 | MI0003249 | >hsa-mir-1470 | MI0007075 |
| >mmu-mir-206 | MI0000249 | >tni-mir-204b | MI0003251 | >hsa-mir-1471 | MI0007076 |
| >mmu-mir-207 | MI0000250 | >fru-mir-458 | MI0003252 | >ssc-mir-99b | MI0007077 |
| >hsa-mir-208a | MI0000251 | >tni-mir-458 | MI0003253 | >oan-mir-1421ag-2 | MI0007078 |
| >hsa-mir-129-1 | MI0000252 | >fru-mir-489 | MI0003254 | >odi-mir-1468 | MI0007079 |
| >hsa-mir-148a | MI0000253 | >tni-mir-489 | MI0003255 | >odi-mir-1469 | MI0007080 |
| >hsa-mir-30c-2 | MI0000254 | >dre-mir-489 | MI0003256 | >odi-mir-1470 | MI0007081 |
| >hsa-mir-30d | MI0000255 | >fru-mir-192 | MI0003257 | >odi-mir-1471 | MI0007082 |
| >mmu-mir-122 | MI0000256 | >tni-mir-192 | MI0003258 | >odi-mir-1472 | MI0007083 |
| >mmu-mir-143 | MI0000257 | >fru-mir-194 | MI0003259 | >odi-mir-1473 | MI0007084 |
| >mmu-mir-30e | MI0000259 | >tni-mir-194 | MI0003260 | >odi-mir-1474 | MI0007085 |
| >hsa-mir-139 | MI0000261 | >fru-mir-142b | MI0003261 | >odi-mir-1475 | MI0007086 |
| >hsa-mir-147 | MI0000262 | >tni-mir-142b | MI0003262 | >odi-mir-1476 | MI0007087 |
| >hsa-mir-7-1 | MI0000263 | >fru-mir-30b | MI0003263 | >odi-mir-1477 | MI0007088 |
| >hsa-mir-7-2 | MI0000264 | >tni-mir-30b | MI0003264 | >odi-mir-1478 | MI0007089 |
| >hsa-mir-7-3 | MI0000265 | >fru-mir-30d | MI0003265 | >odi-mir-1479 | MI0007090 |
| >hsa-mir-10a | MI0000266 | >tni-mir-30d | MI0003266 | >odi-mir-1480 | MI0007091 |
| >hsa-mir-10b | MI0000267 | >fru-mir-27b | MI0003267 | >odi-mir-1481 | MI0007092 |
| >hsa-mir-34a | MI0000268 | >tni-mir-27b | MI0003268 | >odi-mir-1482 | MI0007093 |
| >hsa-mir-181a-2 | MI0000269 | >fru-mir-23b | MI0003269 | >odi-mir-1483 | MI0007094 |
| >hsa-mir-181b-1 | MI0000270 | >tni-mir-23b | MI0003270 | >odi-mir-1484 | MI0007095 |
| >hsa-mir-181c | MI0000271 | >fru-mir-196a-2 | MI0003271 | >odi-mir-1485 | MI0007096 |
| >hsa-mir-182 | MI0000272 | >tni-mir-196a-2 | MI0003272 | >odi-mir-1486 | MI0007097 |
| >hsa-mir-183 | MI0000273 | >fru-mir-126 | MI0003273 | >odi-mir-1487 | MI0007098 |
| >hsa-mir-187 | MI0000274 | >tni-mir-126 | MI0003274 | >odi-mir-1489 | MI0007099 |
| >hsa-mir-196a-2 | MI0000279 | >fru-mir-199-3 | MI0003275 | >odi-mir-1490a-1 | MI0007100 |
| >hsa-mir-199a-2 | MI0000281 | >tni-mir-199-3 | MI0003276 | >odi-mir-1490a-2 | MI0007101 |
| >hsa-mir-199b | MI0000282 | >fru-mir-148 | MI0003277 | >odi-mir-1490b | MI0007102 |
| >hsa-mir-203 | MI0000283 | >tni-mir-148 | MI0003278 | >odi-mir-1491 | MI0007103 |
| >hsa-mir-204 | MI0000284 | >fru-mir-10b-1 | MI0003279 | >odi-mir-1492 | MI0007104 |
| >hsa-mir-205 | MI0000285 | >tni-mir-10b-2 | MI0003280 | >odi-mir-1493-1 | MI0007105 |
| >hsa-mir-210 | MI0000286 | >fru-mir-205 | MI0003281 | >odi-mir-1493-2 | MI0007106 |
| >hsa-mir-211 | MI0000287 | >tni-mir-205 | MI0003282 | >odi-mir-1494 | MI0007107 |
| >hsa-mir-212 | MI0000288 | >fru-mir-365 | MI0003283 | >odi-mir-1495 | MI0007108 |
| >hsa-mir-181a-1 | MI0000289 | >tni-mir-365 | MI0003284 | >odi-mir-1496 | MI0007109 |
| >hsa-mir-214 | MI0000290 | >fru-mir-193 | MI0003285 | >odi-mir-1497a | MI0007110 |
| >hsa-mir-215 | MI0000291 | >tni-mir-193 | MI0003286 | >odi-mir-1497b | MI0007111 |
| >hsa-mir-216a | MI0000292 | >fru-mir-124-1 | MI0003287 | >odi-mir-1497c | MI0007112 |
| >hsa-mir-217 | MI0000293 | >tni-mir-124-1 | MI0003288 | >odi-mir-1497d-1 | MI0007113 |
| >hsa-mir-218-1 | MI0000294 | >fru-mir-217 | MI0003289 | >odi-mir-1497d-2 | MI0007114 |
| >hsa-mir-218-2 | MI0000295 | >tni-mir-217 | MI0003290 | >odi-mir-1497e | MI0007115 |
| >hsa-mir-219-1 | MI0000296 | >fru-mir-216a | MI0003291 | >odi-mir-1497f-1 | MI0007116 |
| >hsa-mir-220a | MI0000297 | >tni-mir-216a | MI0003292 | >odi-mir-1497f-2 | MI0007117 |
| >hsa-mir-221 | MI0000298 | >fru-mir-216b | MI0003293 | >odi-mir-1497g | MI0007118 |
| >hsa-mir-222 | MI0000299 | >tni-mir-216b | MI0003294 | >odi-mir-1497h | MI0007119 |
| >hsa-mir-223 | MI0000300 | >fru-let-7i | MI0003295 | >odi-mir-1498 | MI0007120 |
| >hsa-mir-224 | MI0000301 | >tni-let-7i | MI0003296 | >odi-mir-1499 | MI0007121 |
| >cel-mir-124 | MI0000302 | >fru-mir-10b-2 | MI0003297 | >odi-mir-1500 | MI0007122 |
| >cel-mir-228 | MI0000303 | >tni-mir-10b-1 | MI0003298 | >odi-mir-1501 | MI0007123 |
| >cel-mir-229 | MI0000304 | >fru-mir-196a-1 | MI0003299 | >odi-mir-1502 | MI0007124 |
| >cel-mir-230 | MI0000305 | >tni-mir-196a-1 | MI0003300 | >odi-mir-1503 | MI0007125 |
| >cel-mir-231 | MI0000306 | >fru-mir-429 | MI0003301 | >odi-mir-1504 | MI0007126 |
| >cel-mir-232 | MI0000307 | >tni-mir-429 | MI0003302 | >odi-mir-1505 | MI0007127 |
| >cel-mir-233 | MI0000308 | >fru-mir-200a | MI0003303 | >odi-mir-1506 | MI0007128 |
| >cel-mir-234 | MI0000309 | >tni-mir-200a | MI0003304 | >cin-mir-1473 | MI0007129 |
| >cel-mir-235 | MI0000310 | >fru-mir-200b | MI0003305 | >cin-mir-1497 | MI0007130 |
| >cel-mir-236 | MI0000311 | >tni-mir-200b | MI0003306 | >csa-mir-1473 | MI0007131 |
| >cel-mir-237 | MI0000312 | >fru-mir-218b | MI0003307 | >csa-mir-1497 | MI0007132 |
| >cel-mir-238 | MI0000313 | >tni-mir-218b | MI0003308 | >odi-mir-1a | MI0007133 |
| >cel-mir-239a | MI0000314 | >fru-mir-301 | MI0003309 | >odi-mir-1b | MI0007134 |
| >cel-mir-239b | MI0000315 | >tni-mir-301 | MI0003310 | >odi-mir-1c | MI0007135 |
| >cel-mir-240 | MI0000316 | >fru-mir-130 | MI0003311 | >odi-let-7a | MI0007136 |
| >cel-mir-241 | MI0000317 | >tni-mir-130 | MI0003312 | >odi-let-7b | MI0007137 |
| >cel-mir-242 | MI0000318 | >fru-let-7a-2 | MI0003313 | >odi-let-7c | MI0007138 |
| >cel-mir-243 | MI0000319 | >tni-let-7a-2 | MI0003314 | >odi-let-7d | MI0007139 |
| >cel-mir-244 | MI0000320 | >fru-mir-122 | MI0003315 | >odi-mir-7-1 | MI0007140 |
| >cel-mir-245 | MI0000321 | >tni-mir-122 | MI0003316 | >odi-mir-7-2 | MI0007141 |
| >cel-mir-246 | MI0000322 | >fru-mir-218a-1 | MI0003317 | >odi-mir-31 | MI0007142 |
| >cel-mir-247 | MI0000323 | >tni-mir-218a-1 | MI0003318 | >odi-mir-92a | MI0007143 |
| >cel-mir-248 | MI0000324 | >fru-let-7d | MI0003319 | >odi-mir-92b | MI0007144 |
| >cel-mir-249 | MI0000325 | >tni-let-7d | MI0003320 | >odi-mir-124a | MI0007145 |
| >cel-mir-250 | MI0000326 | >fru-mir-9-2 | MI0003321 | >odi-mir-124b | MI0007146 |
| >cel-mir-251 | MI0000327 | >tni-mir-9-2 | MI0003322 | >odi-mir-219 | MI0007147 |
| >cel-mir-252 | MI0000328 | >fru-mir-460 | MI0003323 | >odi-mir-281 | MI0007148 |
| >cel-mir-253 | MI0000329 | >tni-mir-460 | MI0003324 | >cin-let-7a-1 | MI0007149 |
| >cel-mir-254 | MI0000330 | >fru-mir-21 | MI0003325 | >cin-let-7a-2 | MI0007150 |
| >cel-mir-255 | MI0000331 | >tni-mir-21 | MI0003326 | >cin-let-7b | MI0007151 |
| >cel-mir-256 | MI0000333 | >fru-mir-153b | MI0003327 | >cin-let-7c | MI0007152 |
| >cel-mir-257 | MI0000334 | >tni-mir-153b | MI0003328 | >cin-let-7d | MI0007153 |
| >cel-mir-258 | MI0000335 | >fru-mir-199-2 | MI0003329 | >cin-let-7e | MI0007154 |
| >cel-mir-259 | MI0000336 | >tni-mir-199-2 | MI0003330 | >cin-mir-7 | MI0007155 |
| >cel-mir-260 | MI0000337 | >fru-mir-181b-1 | MI0003331 | >cin-mir-31 | MI0007156 |
| >cel-mir-261 | MI0000338 | >tni-mir-181b-1 | MI0003332 | >cin-mir-33 | MI0007157 |
| >cel-mir-262 | MI0000339 | >fru-mir-181a-1 | MI0003333 | >cin-mir-34 | MI0007158 |
| >hsa-mir-200b | MI0000342 | >tni-mir-181a-1 | MI0003334 | >cin-mir-78 | MI0007159 |
| >dme-mir-263a | MI0000343 | >fru-mir-338 | MI0003335 | >cin-mir-92a | MI0007160 |
| >cel-mir-264 | MI0000344 | >tni-mir-338 | MI0003336 | >cin-mir-92b | MI0007161 |
| >cel-mir-265 | MI0000345 | >fru-mir-7 | MI0003337 | >cin-mir-92c | MI0007162 |
| >cel-mir-266 | MI0000346 | >tni-mir-7 | MI0003338 | >cin-mir-101 | MI0007163 |
| >cel-mir-267 | MI0000347 | >fru-mir-153a | MI0003339 | >cin-mir-124-1 | MI0007164 |
| >cel-mir-268 | MI0000348 | >tni-mir-153a | MI0003340 | >cin-mir-124-2 | MI0007165 |
| >cel-mir-269 | MI0000349 | >fru-let-7j | MI0003341 | >cin-mir-126 | MI0007166 |
| >cel-mir-270 | MI0000350 | >tni-let-7j | MI0003342 | >cin-mir-133 | MI0007167 |
| >cel-mir-271 | MI0000351 | >dre-let-7j | MI0003343 | >cin-mir-141 | MI0007168 |
| >cel-mir-272 | MI0000352 | >fru-let-7b | MI0003344 | >cin-mir-153 | MI0007169 |
| >cel-mir-273 | MI0000353 | >tni-let-7b | MI0003345 | >cin-mir-155 | MI0007170 |
| >dme-mir-184 | MI0000354 | >fru-mir-128-2 | MI0003346 | >cin-mir-181 | MI0007171 |
| >dme-mir-274 | MI0000355 | >tni-mir-128-2 | MI0003347 | >cin-mir-183 | MI0007172 |
| >dme-mir-275 | MI0000356 | >fru-mir-144 | MI0003348 | >cin-mir-184 | MI0007173 |
| >dme-mir-92a | MI0000357 | >tni-mir-144 | MI0003349 | >cin-mir-199 | MI0007174 |
| >dme-mir-219 | MI0000358 | >fru-mir-30c | MI0003350 | >cin-mir-200 | MI0007175 |
| >dme-mir-276a | MI0000359 | >tni-mir-30c | MI0003351 | >cin-mir-216 | MI0007176 |
| >dme-mir-277 | MI0000360 | >fru-mir-140 | MI0003352 | >cin-mir-217 | MI0007177 |
| >dme-mir-278 | MI0000361 | >tni-mir-140 | MI0003353 | >cin-mir-219 | MI0007178 |
| >dme-mir-133 | MI0000362 | >fru-mir-124-2 | MI0003354 | >cin-mir-281 | MI0007179 |
| >dme-mir-279 | MI0000363 | >tni-mir-124-2 | MI0003355 | >cin-mir-672 | MI0007180 |
| >dme-mir-33 | MI0000364 | >fru-mir-214 | MI0003356 | >csa-let-7a | MI0007181 |
| >dme-mir-280 | MI0000365 | >tni-mir-214 | MI0003357 | >csa-let-7b | MI0007182 |
| >dme-mir-281-1 | MI0000366 | >fru-mir-199-1 | MI0003358 | >csa-let-7c-1 | MI0007183 |
| >dme-mir-282 | MI0000367 | >tni-mir-199-1 | MI0003359 | >csa-let-7c-2 | MI0007184 |
| >dme-mir-283 | MI0000368 | >fru-mir-455 | MI0003360 | >csa-let-7d | MI0007185 |
| >dme-mir-284 | MI0000369 | >tni-mir-455 | MI0003361 | >csa-mir-7 | MI0007186 |
| >dme-mir-281-2 | MI0000370 | >fru-mir-135b | MI0003362 | >csa-mir-31 | MI0007187 |
| >dme-mir-34 | MI0000371 | >tni-mir-135b | MI0003363 | >csa-mir-34 | MI0007188 |
| >dme-mir-124 | MI0000373 | >dre-mir-135b | MI0003364 | >csa-mir-92a | MI0007189 |
| >dme-mir-79 | MI0000374 | >fru-mir-196b | MI0003365 | >csa-mir-92b | MI0007190 |
| >dme-mir-276b | MI0000375 | >tni-mir-196b | MI0003366 | >csa-mir-92c | MI0007191 |
| >dme-mir-210 | MI0000376 | >fru-mir-129-1 | MI0003367 | >csa-mir-124-1 | MI0007192 |
| >dme-mir-285 | MI0000377 | >tni-mir-129-1 | MI0003368 | >csa-mir-124-2 | MI0007193 |
| >dme-mir-100 | MI0000378 | >fru-mir-26 | MI0003369 | >csa-mir-126 | MI0007194 |
| >dme-mir-92b | MI0000379 | >tni-mir-26 | MI0003370 | >csa-mir-133 | MI0007195 |
| >dme-mir-286 | MI0000380 | >fru-mir-101b | MI0003371 | >csa-mir-141 | MI0007196 |
| >dme-mir-287 | MI0000381 | >tni-mir-101b | MI0003372 | >csa-mir-153 | MI0007197 |
| >dme-mir-87 | MI0000382 | >fru-mir-9-4 | MI0003373 | >csa-mir-155 | MI0007198 |
| >dme-mir-263b | MI0000383 | >tni-mir-9-4 | MI0003374 | >csa-mir-183 | MI0007199 |
| >dme-mir-288 | MI0000384 | >fru-mir-181b-2 | MI0003375 | >csa-mir-200 | MI0007200 |
| >dme-mir-289 | MI0000385 | >tni-mir-181b-2 | MI0003376 | >csa-mir-216a | MI0007201 |
| >dme-bantam | MI0000387 | >fru-mir-103 | MI0003377 | >csa-mir-216b | MI0007202 |
| >mmu-mir-290 | MI0000388 | >tni-mir-103 | MI0003378 | >csa-mir-217 | MI0007203 |
| >mmu-mir-291a | MI0000389 | >fru-mir-107 | MI0003379 | >csa-mir-219 | MI0007204 |
| >mmu-mir-292 | MI0000390 | >tni-mir-107 | MI0003380 | >csa-mir-281 | MI0007205 |
| >mmu-mir-293 | MI0000391 | >fru-mir-22b | MI0003381 | >gma-MIR159b | MI0007206 |
| >mmu-mir-294 | MI0000392 | >tni-mir-22b | MI0003382 | >gma-MIR159c | MI0007207 |
| >mmu-mir-295 | MI0000393 | >fru-mir-212 | MI0003383 | >gma-MIR162 | MI0007208 |
| >mmu-mir-296 | MI0000394 | >tni-mir-212 | MI0003384 | >gma-MIR164 | MI0007209 |
| >mmu-mir-297a-1 | MI0000395 | >dre-mir-212 | MI0003385 | >gma-MIR167c | MI0007210 |
| >mmu-mir-297a-2 | MI0000397 | >tni-mir-181a-2 | MI0003386 | >gma-MIR169b | MI0007211 |
| >mmu-mir-298 | MI0000398 | >dre-mir-181a-2 | MI0003387 | >gma-MIR169c | MI0007212 |
| >mmu-mir-299 | MI0000399 | >fru-mir-135a | MI0003388 | >gma-MIR171a | MI0007213 |
| >mmu-mir-300 | MI0000400 | >tni-mir-135a | MI0003389 | >gma-MIR390a | MI0007214 |
| >mmu-mir-301a | MI0000401 | >fru-mir-218a-2 | MI0003390 | >gma-MIR390b | MI0007215 |
| >mmu-mir-302a | MI0000402 | >tni-mir-218a-2 | MI0003391 | >gma-MIR393 | MI0007216 |
| >mmu-mir-34c | MI0000403 | >fru-mir-29a-1 | MI0003392 | >gma-MIR171b | MI0007217 |
| >mmu-mir-34b | MI0000404 | >tni-mir-29a-1 | MI0003393 | >gma-MIR482 | MI0007218 |
| >mmu-let-7d | MI0000405 | >dre-mir-29a-2 | MI0003394 | >gma-MIR1507 | MI0007219 |
| >mmu-mir-106a | MI0000406 | >fru-mir-29b-2 | MI0003395 | >gma-MIR1508 | MI0007220 |
| >mmu-mir-106b | MI0000407 | >tni-mir-29b-2 | MI0003396 | >gma-MIR1509 | MI0007221 |
| >mmu-mir-130b | MI0000408 | >fru-mir-133 | MI0003397 | >gma-MIR1510 | MI0007222 |
| >dme-mir-303 | MI0000409 | >tni-mir-133 | MI0003398 | >gma-MIR1511 | MI0007223 |
| >dme-mir-31b | MI0000410 | >fru-mir-132 | MI0003399 | >gma-MIR1512 | MI0007224 |
| >dme-mir-304 | MI0000411 | >tni-mir-132 | MI0003400 | >gma-MIR1513 | MI0007225 |
| >dme-mir-305 | MI0000412 | >fru-mir-125b | MI0003401 | >gma-MIR1514a | MI0007226 |
| >dme-mir-9c | MI0000413 | >tni-mir-125b | MI0003402 | >gma-MIR1514b | MI0007227 |
| >dme-mir-306 | MI0000414 | >fru-mir-29a-2 | MI0003403 | >gma-MIR1515 | MI0007228 |
| >dme-mir-9b | MI0000415 | >tni-mir-29a-2 | MI0003404 | >gma-MIR1516 | MI0007229 |
| >dme-let-7 | MI0000416 | >fru-mir-29b-1 | MI0003405 | >gma-MIR1517 | MI0007230 |
| >dme-mir-125 | MI0000417 | >tni-mir-29b-1 | MI0003406 | >gma-MIR1518 | MI0007231 |
| >dme-mir-307 | MI0000418 | >fru-let-7a-1 | MI0003407 | >gma-MIR1519 | MI0007232 |
| >dme-mir-308 | MI0000419 | >tni-let-7a-1 | MI0003408 | >gma-MIR1520d | MI0007233 |
| >dme-mir-31a | MI0000420 | >fru-mir-100 | MI0003409 | >gma-MIR1536 | MI0007234 |
| >dme-mir-309 | MI0000421 | >tni-mir-100 | MI0003410 | >gma-MIR1520a | MI0007235 |
| >dme-mir-310 | MI0000422 | >fru-mir-219-1 | MI0003411 | >gma-MIR1521 | MI0007236 |
| >dme-mir-311 | MI0000423 | >tni-mir-219-1 | MI0003412 | >gma-MIR1522 | MI0007237 |
| >dme-mir-312 | MI0000424 | >fru-mir-24-1 | MI0003413 | >gma-MIR1523 | MI0007240 |
| >dme-mir-313 | MI0000425 | >tni-mir-24-1 | MI0003414 | >gma-MIR1524 | MI0007241 |
| >dme-mir-314 | MI0000426 | >fru-mir-23a-1 | MI0003415 | >gma-MIR1525 | MI0007242 |
| >dme-mir-315 | MI0000427 | >tni-mir-23a-1 | MI0003416 | >gma-MIR1520b | MI0007243 |
| >dme-mir-316 | MI0000428 | >fru-mir-27e | MI0003417 | >gma-MIR1526 | MI0007244 |
| >dme-mir-317 | MI0000429 | >tni-mir-27e | MI0003418 | >gma-MIR1527 | MI0007245 |
| >dme-mir-318 | MI0000430 | >fru-mir-375 | MI0003419 | >gma-MIR1528 | MI0007246 |
| >dme-mir-2c | MI0000431 | >tni-mir-375 | MI0003420 | >gma-MIR1529 | MI0007248 |
| >dme-mir-iab-4 | MI0000432 | >fru-mir-128-1 | MI0003421 | >gma-MIR1530 | MI0007249 |
| >hsa-let-7g | MI0000433 | >tni-mir-128-1 | MI0003422 | >gma-MIR1531 | MI0007250 |
| >hsa-let-7i | MI0000434 | >fru-mir-1 | MI0003423 | >gma-MIR1532 | MI0007251 |
| >hsa-mir-1-2 | MI0000437 | >tni-mir-1 | MI0003424 | >gma-MIR1520c | MI0007252 |
| >hsa-mir-15b | MI0000438 | >fru-mir-202 | MI0003425 | >gma-MIR1533 | MI0007253 |
| >hsa-mir-23b | MI0000439 | >tni-mir-202 | MI0003426 | >gma-MIR1534 | MI0007254 |
| >hsa-mir-27b | MI0000440 | >fru-mir-203 | MI0003427 | >gma-MIR1535 | MI0007257 |
| >hsa-mir-30b | MI0000441 | >tni-mir-203 | MI0003428 | >hsa-mir-1537 | MI0007258 |
| >hsa-mir-122 | MI0000442 | >fru-mir-23a-3 | MI0003429 | >hsa-mir-1538 | MI0007259 |
| >hsa-mir-124-1 | MI0000443 | >tni-mir-23a-3 | MI0003430 | >hsa-mir-1539 | MI0007260 |
| >hsa-mir-124-2 | MI0000444 | >fru-mir-27c | MI0003431 | >hsa-mir-103-1-as | MI0007261 |
| >hsa-mir-124-3 | MI0000445 | >tni-mir-27c | MI0003432 | >hsa-mir-103-2-as | MI0007262 |
| >hsa-mir-125b-1 | MI0000446 | >fru-mir-24-2 | MI0003433 | >mdo-mir-1540 | MI0007263 |
| >hsa-mir-128-1 | MI0000447 | >tni-mir-24-2 | MI0003434 | >mdo-mir-1541 | MI0007264 |
| >hsa-mir-130a | MI0000448 | >fru-mir-138 | MI0003435 | >mdo-mir-1542-1 | MI0007265 |
| >hsa-mir-132 | MI0000449 | >tni-mir-138 | MI0003436 | >mdo-mir-1542-2 | MI0007266 |
| >hsa-mir-133a-1 | MI0000450 | >fru-mir-129-2 | MI0003437 | >mdo-mir-1543 | MI0007267 |
| >hsa-mir-133a-2 | MI0000451 | >tni-mir-129-2 | MI0003438 | >mdo-mir-340 | MI0007268 |
| >hsa-mir-135a-1 | MI0000452 | >fru-mir-125a | MI0003439 | >mdo-mir-1544 | MI0007269 |
| >hsa-mir-135a-2 | MI0000453 | >tni-mir-125a | MI0003440 | >mdo-mir-1545 | MI0007270 |
| >hsa-mir-137 | MI0000454 | >fru-mir-17-2 | MI0003441 | >mdo-mir-1546 | MI0007271 |
| >hsa-mir-138-2 | MI0000455 | >tni-mir-17-2 | MI0003442 | >mdo-mir-1547 | MI0007272 |
| >hsa-mir-140 | MI0000456 | >fru-mir-20 | MI0003443 | >mdo-mir-1548 | MI0007273 |
| >hsa-mir-141 | MI0000457 | >tni-mir-20 | MI0003444 | >mdo-mir-1549 | MI0007274 |
| >hsa-mir-142 | MI0000458 | >fru-mir-92-1 | MI0003445 | >gga-mir-1550 | MI0007275 |
| >hsa-mir-143 | MI0000459 | >tni-mir-92-1 | MI0003446 | >gga-mir-1551 | MI0007276 |
| >hsa-mir-144 | MI0000460 | >fru-mir-15b | MI0003447 | >gga-mir-1552 | MI0007277 |
| >hsa-mir-145 | MI0000461 | >tni-mir-15b | MI0003448 | >gga-mir-1553 | MI0007278 |
| >hsa-mir-152 | MI0000462 | >fru-mir-10c | MI0003449 | >gga-mir-1554 | MI0007279 |
| >hsa-mir-153-1 | MI0000463 | >tni-mir-10c | MI0003450 | >gga-mir-1555 | MI0007280 |
| >hsa-mir-153-2 | MI0000464 | >fru-mir-9-3 | MI0003451 | >gga-mir-1556 | MI0007281 |
| >hsa-mir-191 | MI0000465 | >tni-mir-9-3 | MI0003452 | >gga-mir-1557 | MI0007282 |
| >hsa-mir-9-1 | MI0000466 | >fru-mir-183 | MI0003453 | >gga-mir-1558 | MI0007283 |
| >hsa-mir-9-2 | MI0000467 | >tni-mir-183 | MI0003454 | >gga-mir-1559 | MI0007284 |
| >hsa-mir-9-3 | MI0000468 | >fru-mir-96 | MI0003455 | >gga-mir-1560 | MI0007285 |
| >hsa-mir-125a | MI0000469 | >tni-mir-96 | MI0003456 | >gga-mir-1561 | MI0007286 |
| >hsa-mir-125b-2 | MI0000470 | >fru-mir-182 | MI0003457 | >gga-mir-1562 | MI0007287 |
| >hsa-mir-126 | MI0000471 | >tni-mir-182 | MI0003458 | >gga-mir-1563 | MI0007288 |
| >hsa-mir-127 | MI0000472 | >fru-mir-210 | MI0003459 | >gga-mir-1564 | MI0007289 |
| >hsa-mir-129-2 | MI0000473 | >tni-mir-210 | MI0003460 | >gga-mir-1565 | MI0007290 |
| >hsa-mir-134 | MI0000474 | >fru-let-7g | MI0003461 | >gga-mir-1566 | MI0007291 |
| >hsa-mir-136 | MI0000475 | >tni-let-7g | MI0003462 | >gga-mir-1567 | MI0007292 |
| >hsa-mir-138-1 | MI0000476 | >fru-mir-22a | MI0003463 | >gga-mir-1568 | MI0007293 |
| >hsa-mir-146a | MI0000477 | >tni-mir-22a | MI0003464 | >gga-mir-1569-1 | MI0007294 |
| >hsa-mir-149 | MI0000478 | >fru-mir-204 | MI0003465 | >gga-mir-1569-2 | MI0007295 |
| >hsa-mir-150 | MI0000479 | >tni-mir-204a | MI0003466 | >gga-mir-1570 | MI0007296 |
| >hsa-mir-154 | MI0000480 | >fru-mir-152 | MI0003467 | >gga-mir-1571 | MI0007297 |
| >hsa-mir-184 | MI0000481 | >tni-mir-152 | MI0003468 | >gga-mir-1572 | MI0007298 |
| >hsa-mir-185 | MI0000482 | >fru-mir-15a | MI0003469 | >gga-mir-1573 | MI0007299 |
| >hsa-mir-186 | MI0000483 | >tni-mir-15a | MI0003470 | >gga-mir-1574 | MI0007300 |
| >hsa-mir-188 | MI0000484 | >fru-mir-16 | MI0003471 | >gga-mir-1575 | MI0007301 |
| >hsa-mir-190 | MI0000486 | >tni-mir-16 | MI0003472 | >gga-mir-1576 | MI0007302 |
| >hsa-mir-193a | MI0000487 | >fru-mir-190 | MI0003473 | >gga-mir-1577 | MI0007303 |
| >hsa-mir-194-1 | MI0000488 | >tni-mir-190 | MI0003474 | >gga-mir-1578 | MI0007304 |
| >hsa-mir-195 | MI0000489 | >fru-mir-181a-2 | MI0003475 | >gga-mir-1579 | MI0007305 |
| >hsa-mir-206 | MI0000490 | >mmu-mir-489 | MI0003476 | >gga-mir-1580 | MI0007306 |
| >cbr-let-7 | MI0000491 | >rno-mir-489 | MI0003477 | >gga-mir-1581 | MI0007307 |
| >cbr-lin-4 | MI0000492 | >rno-mir-383 | MI0003478 | >gga-mir-1582 | MI0007308 |
| >cbr-mir-1 | MI0000493 | >rno-mir-207 | MI0003479 | >gga-mir-1583 | MI0007309 |
| >cbr-mir-34 | MI0000494 | >rno-mir-501 | MI0003480 | >gga-mir-1584 | MI0007310 |
| >cbr-mir-42 | MI0000495 | >rno-mir-361 | MI0003481 | >gga-mir-1354 | MI0007311 |
| >cbr-mir-43 | MI0000496 | >rno-mir-215 | MI0003482 | >gga-mir-1585 | MI0007312 |
| >cbr-mir-44 | MI0000497 | >rno-mir-224 | MI0003483 | >gga-mir-1586 | MI0007313 |
| >cbr-mir-45-1 | MI0000498 | >mmu-mir-483 | MI0003484 | >gga-mir-1587 | MI0007314 |
| >cbr-mir-45-2 | MI0000499 | >rno-mir-483 | MI0003485 | >gga-mir-1588 | MI0007315 |
| >cbr-mir-46 | MI0000500 | >rno-mir-370 | MI0003486 | >gga-mir-1589 | MI0007316 |
| >cbr-mir-47 | MI0000501 | >rno-mir-377 | MI0003487 | >gga-mir-1590 | MI0007317 |
| >cbr-mir-48 | MI0000502 | >rno-mir-412 | MI0003488 | >gga-mir-1591 | MI0007318 |
| >cbr-mir-49 | MI0000503 | >rno-mir-1 | MI0003489 | >gga-mir-1592 | MI0007319 |
| >cbr-mir-50 | MI0000504 | >rno-mir-133b | MI0003490 | >gga-mir-1593 | MI0007320 |
| >cbr-mir-52 | MI0000505 | >mmu-mir-484 | MI0003491 | >gga-mir-1594 | MI0007321 |
| >cbr-mir-57 | MI0000507 | >mmu-mir-485 | MI0003492 | >gga-mir-1595 | MI0007322 |
| >cbr-mir-58 | MI0000508 | >mmu-mir-486 | MI0003493 | >gga-mir-1596 | MI0007323 |
| >cbr-mir-60 | MI0000509 | >ppt-MIR390a | MI0003494 | >gga-mir-1597 | MI0007324 |
| >cbr-mir-67 | MI0000510 | >ppt-MIR390b | MI0003495 | >gga-mir-1598 | MI0007325 |
| >cbr-mir-71 | MI0000511 | >ppt-MIR319a | MI0003496 | >gga-mir-1599 | MI0007326 |
| >cbr-mir-73 | MI0000512 | >ppt-MIR319b | MI0003497 | >gga-mir-1600 | MI0007327 |
| >cbr-mir-74 | MI0000513 | >ppt-MIR319c | MI0003498 | >gga-mir-1601 | MI0007328 |
| >cbr-mir-75 | MI0000514 | >ppt-MIR319d | MI0003499 | >gga-mir-1602 | MI0007329 |
| >cbr-mir-77 | MI0000515 | >ppt-MIR533a | MI0003500 | >gga-mir-1603 | MI0007330 |
| >cbr-mir-79 | MI0000517 | >ppt-MIR534a | MI0003501 | >gga-mir-1604 | MI0007331 |
| >cbr-mir-80 | MI0000518 | >ppt-MIR535a | MI0003502 | >gga-mir-1605 | MI0007332 |
| >cbr-mir-81 | MI0000519 | >ppt-MIR535b | MI0003503 | >gga-mir-1606 | MI0007333 |
| >cbr-mir-82 | MI0000520 | >ppt-MIR535c | MI0003504 | >gga-mir-1607 | MI0007334 |
| >cbr-mir-85 | MI0000521 | >osa-MIR535 | MI0003505 | >gga-mir-1608 | MI0007335 |
| >cbr-mir-86 | MI0000522 | >ppt-MIR156a | MI0003506 | >gga-mir-1609-1 | MI0007336 |
| >cbr-mir-87 | MI0000523 | >ppt-MIR536a | MI0003507 | >gga-mir-1609-2 | MI0007337 |
| >cbr-mir-90 | MI0000524 | >ppt-MIR537a | MI0003508 | >gga-mir-1610 | MI0007338 |
| >cbr-mir-124 | MI0000525 | >ppt-MIR537b | MI0003509 | >gga-mir-1611 | MI0007339 |
| >cbr-mir-228 | MI0000526 | >ppt-MIR538a | MI0003510 | >gga-mir-1612 | MI0007340 |
| >cbr-mir-230 | MI0000527 | >ppt-MIR538b | MI0003511 | >gga-mir-1613-1 | MI0007341 |
| >cbr-mir-232 | MI0000528 | >ppt-MIR538c | MI0003512 | >gga-mir-1613-2 | MI0007342 |
| >cbr-mir-233 | MI0000530 | >hsa-mir-455 | MI0003513 | >gga-mir-1614 | MI0007343 |
| >cbr-mir-234 | MI0000531 | >hsa-mir-539 | MI0003514 | >gga-mir-1615 | MI0007344 |
| >cbr-mir-236 | MI0000532 | >hsa-mir-544 | MI0003515 | >gga-mir-1616 | MI0007345 |
| >cbr-mir-241 | MI0000533 | >hsa-mir-545 | MI0003516 | >gga-mir-1617 | MI0007346 |
| >cbr-mir-244 | MI0000534 | >mmu-mir-546 | MI0003517 | >gga-mir-1618 | MI0007347 |
| >cbr-mir-245 | MI0000535 | >mmu-mir-540 | MI0003518 | >gga-mir-1619 | MI0007348 |
| >cbr-mir-248 | MI0000536 | >mmu-mir-543 | MI0003519 | >gga-mir-1620 | MI0007349 |
| >cbr-mir-250 | MI0000537 | >mmu-mir-539 | MI0003520 | >gga-mir-1621 | MI0007350 |
| >cbr-mir-251 | MI0000538 | >mmu-mir-541 | MI0003521 | >gga-mir-1622 | MI0007351 |
| >cbr-mir-252 | MI0000539 | >mmu-mir-542 | MI0003522 | >gga-mir-1623 | MI0007352 |
| >cbr-mir-259 | MI0000540 | >mmu-mir-547 | MI0003523 | >gga-mir-1624 | MI0007353 |
| >cbr-mir-268 | MI0000541 | >rno-mir-540 | MI0003524 | >gga-mir-1625 | MI0007354 |
| >hsa-mir-320a | MI0000542 | >rno-mir-543 | MI0003525 | >gga-mir-1626 | MI0007355 |
| >ath-MIR319a | MI0000544 | >rno-mir-539 | MI0003526 | >gga-mir-1627 | MI0007356 |
| >ath-MIR319b | MI0000545 | >rno-mir-541 | MI0003527 | >gga-mir-1628 | MI0007357 |
| >mmu-mir-19b-2 | MI0000546 | >rno-mir-542 | MI0003528 | >gga-mir-1629 | MI0007358 |
| >mmu-mir-30c-1 | MI0000547 | >hsa-mir-376a-2 | MI0003529 | >gga-mir-1630 | MI0007359 |
| >mmu-mir-30c-2 | MI0000548 | >hsa-mir-487b | MI0003530 | >gga-mir-1631 | MI0007360 |
| >mmu-mir-30d | MI0000549 | >mmu-mir-367 | MI0003531 | >gga-mir-1c | MI0007361 |
| >mmu-mir-148a | MI0000550 | >mmu-mir-494 | MI0003532 | >gga-mir-1632 | MI0007362 |
| >mmu-mir-192 | MI0000551 | >mmu-mir-376c | MI0003533 | >gga-mir-1633 | MI0007363 |
| >mmu-mir-196a-1 | MI0000552 | >mmu-mir-487b | MI0003534 | >gga-mir-1634 | MI0007364 |
| >mmu-mir-196a-2 | MI0000553 | >mmu-mir-369 | MI0003535 | >gga-mir-1635 | MI0007365 |
| >mmu-mir-200a | MI0000554 | >mmu-mir-20b | MI0003536 | >gga-mir-1636 | MI0007366 |
| >mmu-mir-208a | MI0000555 | >mmu-mir-450a-2 | MI0003537 | >gga-mir-1637 | MI0007367 |
| >mmu-let-7a-1 | MI0000556 | >mmu-mir-503 | MI0003538 | >gga-mir-1638 | MI0007368 |
| >mmu-let-7a-2 | MI0000557 | >mmu-mir-291b | MI0003539 | >gga-mir-1815 | MI0007369 |
| >mmu-let-7b | MI0000558 | >rno-mir-493 | MI0003540 | >gga-mir-1814 | MI0007370 |
| >mmu-let-7c-1 | MI0000559 | >rno-mir-379 | MI0003541 | >gga-mir-1639 | MI0007371 |
| >mmu-let-7c-2 | MI0000560 | >rno-mir-494 | MI0003542 | >gga-mir-1640 | MI0007372 |
| >mmu-let-7e | MI0000561 | >rno-mir-376c | MI0003543 | >gga-mir-1641 | MI0007373 |
| >mmu-let-7f-1 | MI0000562 | >rno-mir-376b | MI0003544 | >gga-mir-1642 | MI0007374 |
| >mmu-let-7f-2 | MI0000563 | >rno-mir-376a | MI0003545 | >gga-mir-1643 | MI0007375 |
| >mmu-mir-15a | MI0000564 | >rno-mir-381 | MI0003546 | >gga-mir-1644 | MI0007376 |
| >mmu-mir-16-1 | MI0000565 | >rno-mir-487b | MI0003547 | >gga-mir-1645 | MI0007377 |
| >mmu-mir-16-2 | MI0000566 | >rno-mir-382 | MI0003548 | >gga-mir-1646 | MI0007378 |
| >mmu-mir-18a | MI0000567 | >rno-mir-485 | MI0003549 | >gga-mir-1647 | MI0007379 |
| >mmu-mir-20a | MI0000568 | >rno-mir-409 | MI0003550 | >gga-mir-1648 | MI0007380 |
| >mmu-mir-21 | MI0000569 | >rno-mir-369 | MI0003551 | >gga-mir-1649 | MI0007381 |
| >mmu-mir-22 | MI0000570 | >rno-mir-374 | MI0003552 | >gga-mir-1650 | MI0007382 |
| >mmu-mir-23a | MI0000571 | >rno-mir-363 | MI0003553 | >gga-mir-1651 | MI0007383 |
| >mmu-mir-24-2 | MI0000572 | >rno-mir-20b | MI0003554 | >gga-mir-1652 | MI0007384 |
| >mmu-mir-26a-1 | MI0000573 | >rno-mir-503 | MI0003555 | >gga-mir-1653 | MI0007385 |
| >mmu-mir-26b | MI0000575 | >hsa-mir-551a | MI0003556 | >gga-mir-1654-1 | MI0007386 |
| >mmu-mir-29a | MI0000576 | >hsa-mir-552 | MI0003557 | >gga-mir-1654-2 | MI0007387 |
| >mmu-mir-29c | MI0000577 | >hsa-mir-553 | MI0003558 | >gga-mir-1655 | MI0007388 |
| >mmu-mir-27a | MI0000578 | >hsa-mir-554 | MI0003559 | >gga-mir-1656 | MI0007389 |
| >mmu-mir-31 | MI0000579 | >hsa-mir-92b | MI0003560 | >gga-mir-135b | MI0007390 |
| >mmu-mir-92a-2 | MI0000580 | >hsa-mir-555 | MI0003561 | >gga-mir-1657 | MI0007391 |
| >mmu-mir-93 | MI0000581 | >hsa-mir-556 | MI0003562 | >gga-mir-1658 | MI0007392 |
| >mmu-mir-96 | MI0000583 | >hsa-mir-557 | MI0003563 | >gga-mir-1659 | MI0007393 |
| >mmu-mir-34a | MI0000584 | >hsa-mir-558 | MI0003564 | >gga-mir-1660 | MI0007394 |
| >mmu-mir-129-2 | MI0000585 | >hsa-mir-559 | MI0003565 | >gga-mir-1661 | MI0007395 |
| >mmu-mir-98 | MI0000586 | >hsa-mir-561 | MI0003567 | >gga-mir-1662 | MI0007396 |
| >mmu-mir-103-1 | MI0000587 | >hsa-mir-562 | MI0003568 | >gga-mir-1663 | MI0007397 |
| >mmu-mir-103-2 | MI0000588 | >hsa-mir-563 | MI0003569 | >gga-mir-1664 | MI0007398 |
| >rno-mir-322 | MI0000589 | >hsa-mir-564 | MI0003570 | >gga-mir-1665 | MI0007399 |
| >mmu-mir-322 | MI0000590 | >hsa-mir-566 | MI0003572 | >gga-mir-1666 | MI0007400 |
| >rno-mir-323 | MI0000591 | >hsa-mir-567 | MI0003573 | >gga-mir-1667 | MI0007401 |
| >mmu-mir-323 | MI0000592 | >hsa-mir-568 | MI0003574 | >gga-mir-1668 | MI0007402 |
| >rno-mir-301a | MI0000593 | >hsa-mir-551b | MI0003575 | >gga-mir-1669 | MI0007403 |
| >rno-mir-324 | MI0000594 | >hsa-mir-569 | MI0003576 | >gga-mir-1670 | MI0007404 |
| >mmu-mir-324 | MI0000595 | >hsa-mir-570 | MI0003577 | >gga-mir-1671 | MI0007405 |
| >rno-mir-325 | MI0000596 | >hsa-mir-571 | MI0003578 | >gga-mir-1672 | MI0007406 |
| >mmu-mir-325 | MI0000597 | >hsa-mir-572 | MI0003579 | >gga-mir-1673 | MI0007407 |
| >mmu-mir-326 | MI0000598 | >hsa-mir-573 | MI0003580 | >gga-mir-1674 | MI0007408 |
| >rno-mir-326 | MI0000599 | >hsa-mir-574 | MI0003581 | >gga-mir-1675 | MI0007409 |
| >rno-mir-327 | MI0000600 | >hsa-mir-575 | MI0003582 | >gga-mir-1676 | MI0007410 |
| >rno-let-7d | MI0000601 | >hsa-mir-576 | MI0003583 | >gga-mir-1677 | MI0007411 |
| >rno-mir-328 | MI0000602 | >hsa-mir-577 | MI0003584 | >gga-mir-1678 | MI0007412 |
| >mmu-mir-328 | MI0000603 | >hsa-mir-578 | MI0003585 | >gga-mir-1679 | MI0007413 |
| >rno-mir-329 | MI0000604 | >hsa-mir-579 | MI0003586 | >gga-mir-1680 | MI0007414 |
| >mmu-mir-329 | MI0000605 | >hsa-mir-580 | MI0003587 | >gga-mir-1681 | MI0007415 |
| >rno-mir-330 | MI0000606 | >hsa-mir-581 | MI0003588 | >gga-mir-1682 | MI0007416 |
| >mmu-mir-330 | MI0000607 | >hsa-mir-582 | MI0003589 | >gga-mir-1683 | MI0007417 |
| >rno-mir-331 | MI0000608 | >hsa-mir-583 | MI0003590 | >gga-mir-1684 | MI0007418 |
| >mmu-mir-331 | MI0000609 | >hsa-mir-584 | MI0003591 | >gga-mir-1685 | MI0007419 |
| >rno-mir-140 | MI0000611 | >hsa-mir-585 | MI0003592 | >gga-mir-1686 | MI0007420 |
| >rno-mir-335 | MI0000612 | >hsa-mir-548a-1 | MI0003593 | >gga-mir-1687 | MI0007421 |
| >rno-mir-336 | MI0000613 | >hsa-mir-586 | MI0003594 | >gga-mir-1688 | MI0007422 |
| >rno-mir-337 | MI0000614 | >hsa-mir-587 | MI0003595 | >gga-mir-1689 | MI0007423 |
| >mmu-mir-337 | MI0000615 | >hsa-mir-548b | MI0003596 | >gga-mir-1690 | MI0007424 |
| >rno-mir-148b | MI0000616 | >hsa-mir-588 | MI0003597 | >gga-mir-1691 | MI0007425 |
| >mmu-mir-148b | MI0000617 | >hsa-mir-548a-2 | MI0003598 | >gga-mir-199b | MI0007426 |
| >rno-mir-338 | MI0000618 | >hsa-mir-589 | MI0003599 | >gga-mir-1692 | MI0007427 |
| >mmu-mir-338 | MI0000619 | >hsa-mir-550-1 | MI0003600 | >gga-mir-1693 | MI0007428 |
| >rno-mir-339 | MI0000620 | >hsa-mir-550-2 | MI0003601 | >gga-mir-1694 | MI0007429 |
| >mmu-mir-339 | MI0000621 | >hsa-mir-590 | MI0003602 | >gga-mir-1695 | MI0007430 |
| >rno-mir-340 | MI0000622 | >hsa-mir-591 | MI0003603 | >gga-mir-1696 | MI0007431 |
| >mmu-mir-340 | MI0000623 | >hsa-mir-592 | MI0003604 | >gga-mir-1697 | MI0007432 |
| >rno-mir-341 | MI0000624 | >hsa-mir-593 | MI0003605 | >gga-mir-1698 | MI0007433 |
| >mmu-mir-341 | MI0000625 | >hsa-mir-595 | MI0003607 | >gga-mir-1699 | MI0007434 |
| >rno-mir-342 | MI0000626 | >hsa-mir-596 | MI0003608 | >gga-mir-1700 | MI0007435 |
| >mmu-mir-342 | MI0000627 | >hsa-mir-597 | MI0003609 | >gga-mir-1701 | MI0007436 |
| >rno-mir-343 | MI0000628 | >hsa-mir-598 | MI0003610 | >gga-mir-1702 | MI0007437 |
| >rno-mir-344-1 | MI0000629 | >hsa-mir-599 | MI0003611 | >gga-mir-1703 | MI0007438 |
| >mmu-mir-344-1 | MI0000630 | >hsa-mir-548a-3 | MI0003612 | >gga-mir-1704 | MI0007439 |
| >rno-mir-345 | MI0000631 | >hsa-mir-600 | MI0003613 | >gga-mir-1705 | MI0007440 |
| >mmu-mir-345 | MI0000632 | >hsa-mir-601 | MI0003614 | >gga-mir-1706 | MI0007441 |
| >rno-mir-346 | MI0000633 | >hsa-mir-602 | MI0003615 | >gga-mir-1707 | MI0007442 |
| >mmu-mir-346 | MI0000634 | >hsa-mir-603 | MI0003616 | >gga-mir-1813-2 | MI0007443 |
| >rno-mir-347 | MI0000635 | >hsa-mir-604 | MI0003617 | >gga-mir-1708 | MI0007444 |
| >rno-mir-349 | MI0000636 | >hsa-mir-605 | MI0003618 | >gga-mir-449c | MI0007445 |
| >rno-mir-129-2 | MI0000637 | >hsa-mir-606 | MI0003619 | >gga-mir-1709 | MI0007446 |
| >rno-mir-20a | MI0000638 | >hsa-mir-607 | MI0003620 | >gga-mir-1710 | MI0007447 |
| >rno-mir-350 | MI0000639 | >hsa-mir-608 | MI0003621 | >gga-mir-1711 | MI0007448 |
| >mmu-mir-350 | MI0000640 | >hsa-mir-609 | MI0003622 | >gga-mir-1712 | MI0007449 |
| >rno-mir-7a-1 | MI0000641 | >hsa-mir-610 | MI0003623 | >gga-mir-1713 | MI0007450 |
| >rno-mir-351 | MI0000642 | >hsa-mir-611 | MI0003624 | >gga-mir-1714 | MI0007451 |
| >mmu-mir-351 | MI0000643 | >hsa-mir-612 | MI0003625 | >gga-mir-1715 | MI0007452 |
| >rno-mir-352 | MI0000644 | >hsa-mir-613 | MI0003626 | >gga-mir-1716 | MI0007453 |
| >rno-mir-135b | MI0000645 | >hsa-mir-614 | MI0003627 | >gga-mir-1717 | MI0007454 |
| >mmu-mir-135b | MI0000646 | >hsa-mir-615 | MI0003628 | >gga-mir-1718 | MI0007455 |
| >rno-mir-151 | MI0000647 | >hsa-mir-616 | MI0003629 | >gga-mir-1719 | MI0007456 |
| >rno-mir-101b | MI0000648 | >hsa-mir-548c | MI0003630 | >gga-mir-1720 | MI0007457 |
| >mmu-mir-101b | MI0000649 | >hsa-mir-617 | MI0003631 | >gga-mir-1721 | MI0007458 |
| >hsa-mir-200c | MI0000650 | >hsa-mir-618 | MI0003632 | >gga-mir-1722 | MI0007459 |
| >hsa-mir-1-1 | MI0000651 | >hsa-mir-619 | MI0003633 | >gga-mir-1723 | MI0007460 |
| >mmu-mir-1-2 | MI0000652 | >hsa-mir-620 | MI0003634 | >gga-mir-1724 | MI0007461 |
| >osa-MIR156a | MI0000653 | >hsa-mir-621 | MI0003635 | >gga-mir-1725 | MI0007462 |
| >osa-MIR156b | MI0000654 | >hsa-mir-622 | MI0003636 | >gga-mir-1726 | MI0007463 |
| >osa-MIR156c | MI0000655 | >hsa-mir-623 | MI0003637 | >gga-mir-1727-1 | MI0007464 |
| >osa-MIR156d | MI0000656 | >hsa-mir-624 | MI0003638 | >gga-mir-1727-2 | MI0007465 |
| >osa-MIR156e | MI0000657 | >hsa-mir-625 | MI0003639 | >gga-mir-1728 | MI0007466 |
| >osa-MIR156f | MI0000658 | >hsa-mir-626 | MI0003640 | >gga-mir-1813-1 | MI0007467 |
| >osa-MIR156g | MI0000659 | >hsa-mir-627 | MI0003641 | >gga-mir-1729 | MI0007468 |
| >osa-MIR156h | MI0000660 | >hsa-mir-628 | MI0003642 | >gga-mir-1730 | MI0007469 |
| >osa-MIR156i | MI0000661 | >hsa-mir-629 | MI0003643 | >gga-mir-1731 | MI0007470 |
| >osa-MIR156j | MI0000662 | >hsa-mir-630 | MI0003644 | >gga-mir-1732 | MI0007471 |
| >osa-MIR160a | MI0000663 | >hsa-mir-631 | MI0003645 | >gga-mir-1733 | MI0007472 |
| >osa-MIR160b | MI0000664 | >hsa-mir-33b | MI0003646 | >gga-mir-1734 | MI0007473 |
| >osa-MIR160c | MI0000665 | >hsa-mir-632 | MI0003647 | >gga-mir-1735 | MI0007474 |
| >osa-MIR160d | MI0000666 | >hsa-mir-633 | MI0003648 | >gga-mir-1736 | MI0007475 |
| >osa-MIR162a | MI0000667 | >hsa-mir-634 | MI0003649 | >gga-mir-1737 | MI0007476 |
| >osa-MIR164a | MI0000668 | >hsa-mir-635 | MI0003650 | >gga-mir-1738 | MI0007477 |
| >osa-MIR164b | MI0000669 | >hsa-mir-636 | MI0003651 | >gga-mir-1739 | MI0007478 |
| >osa-MIR166a | MI0000670 | >hsa-mir-637 | MI0003652 | >gga-mir-1740 | MI0007479 |
| >osa-MIR166b | MI0000671 | >hsa-mir-638 | MI0003653 | >gga-mir-1741 | MI0007480 |
| >osa-MIR166c | MI0000672 | >hsa-mir-639 | MI0003654 | >gga-mir-1742 | MI0007481 |
| >osa-MIR166d | MI0000673 | >hsa-mir-640 | MI0003655 | >gga-mir-1743 | MI0007482 |
| >osa-MIR166e | MI0000674 | >hsa-mir-641 | MI0003656 | >gga-mir-1744 | MI0007483 |
| >osa-MIR166f | MI0000675 | >hsa-mir-642 | MI0003657 | >gga-mir-1745-1 | MI0007484 |
| >osa-MIR167a | MI0000676 | >hsa-mir-643 | MI0003658 | >gga-mir-1745-2 | MI0007485 |
| >osa-MIR167b | MI0000677 | >hsa-mir-644 | MI0003659 | >gga-mir-1746 | MI0007486 |
| >osa-MIR167c | MI0000678 | >hsa-mir-645 | MI0003660 | >gga-mir-1747 | MI0007487 |
| >osa-MIR169a | MI0000679 | >hsa-mir-646 | MI0003661 | >gga-mir-1748 | MI0007488 |
| >osa-MIR171a | MI0000680 | >hsa-mir-647 | MI0003662 | >gga-mir-1749 | MI0007489 |
| >hsa-mir-155 | MI0000681 | >hsa-mir-648 | MI0003663 | >gga-mir-1816 | MI0007490 |
| >hsa-mir-181b-2 | MI0000683 | >hsa-mir-649 | MI0003664 | >gga-mir-1750 | MI0007491 |
| >mmu-mir-107 | MI0000684 | >hsa-mir-650 | MI0003665 | >gga-mir-1751 | MI0007492 |
| >mmu-mir-10a | MI0000685 | >hsa-mir-651 | MI0003666 | >gga-mir-1752 | MI0007493 |
| >mmu-mir-17 | MI0000687 | >hsa-mir-652 | MI0003667 | >gga-mir-1753-1 | MI0007494 |
| >mmu-mir-19a | MI0000688 | >hsa-mir-548d-1 | MI0003668 | >gga-mir-1753-2 | MI0007495 |
| >mmu-mir-25 | MI0000689 | >hsa-mir-661 | MI0003669 | >gga-mir-1754 | MI0007496 |
| >mmu-mir-28 | MI0000690 | >hsa-mir-662 | MI0003670 | >gga-mir-1755 | MI0007497 |
| >mmu-mir-32 | MI0000691 | >hsa-mir-548d-2 | MI0003671 | >gga-mir-1756a | MI0007498 |
| >mmu-mir-100 | MI0000692 | >hsa-mir-663 | MI0003672 | >gga-mir-1757 | MI0007499 |
| >mmu-mir-139 | MI0000693 | >hsa-mir-449b | MI0003673 | >gga-mir-1758 | MI0007500 |
| >mmu-mir-200c | MI0000694 | >hsa-mir-653 | MI0003674 | >gga-mir-1759 | MI0007501 |
| >mmu-mir-210 | MI0000695 | >hsa-mir-411 | MI0003675 | >gga-mir-1760 | MI0007502 |
| >mmu-mir-212 | MI0000696 | >hsa-mir-654 | MI0003676 | >gga-mir-1761 | MI0007503 |
| >mmu-mir-181a-1 | MI0000697 | >hsa-mir-655 | MI0003677 | >gga-mir-1762 | MI0007504 |
| >mmu-mir-214 | MI0000698 | >hsa-mir-656 | MI0003678 | >gga-mir-1763 | MI0007505 |
| >mmu-mir-216a | MI0000699 | >hsa-mir-549 | MI0003679 | >gga-mir-1764 | MI0007506 |
| >mmu-mir-218-1 | MI0000700 | >hsa-mir-657 | MI0003681 | >gga-mir-1765 | MI0007507 |
| >mmu-mir-218-2 | MI0000701 | >hsa-mir-658 | MI0003682 | >gga-mir-1766-1 | MI0007508 |
| >mmu-mir-219-1 | MI0000702 | >hsa-mir-659 | MI0003683 | >gga-mir-1766-2 | MI0007509 |
| >mmu-mir-223 | MI0000703 | >hsa-mir-660 | MI0003684 | >gga-mir-1767 | MI0007510 |
| >mmu-mir-320 | MI0000704 | >hsa-mir-421 | MI0003685 | >gga-mir-1768 | MI0007511 |
| >mmu-mir-26a-2 | MI0000706 | >hsa-mir-542 | MI0003686 | >gga-mir-1769 | MI0007512 |
| >mmu-mir-33 | MI0000707 | >hcmv-mir-US4 | MI0003687 | >gga-mir-1770 | MI0007513 |
| >mmu-mir-211 | MI0000708 | >hcmv-mir-UL70 | MI0003688 | >gga-mir-1771 | MI0007514 |
| >mmu-mir-221 | MI0000709 | >sv40-mir-S1 | MI0003689 | >gga-mir-1772 | MI0007515 |
| >mmu-mir-222 | MI0000710 | >dre-mir-34b | MI0003690 | >gga-mir-1773 | MI0007516 |
| >mmu-mir-224 | MI0000711 | >dre-mir-31 | MI0003691 | >gga-mir-1774 | MI0007517 |
| >mmu-mir-29b-2 | MI0000712 | >dre-mir-135a | MI0003692 | >gga-mir-1775 | MI0007518 |
| >mmu-mir-199a-2 | MI0000713 | >dre-mir-139 | MI0003693 | >gga-mir-1776 | MI0007519 |
| >mmu-mir-199b | MI0000714 | >gga-mir-9-1 | MI0003694 | >gga-mir-1777 | MI0007520 |
| >mmu-mir-135a-2 | MI0000715 | >gga-mir-146b | MI0003695 | >gga-mir-1778 | MI0007521 |
| >mmu-mir-124-1 | MI0000716 | >gga-mir-147-1 | MI0003696 | >gga-mir-1779 | MI0007522 |
| >mmu-mir-124-2 | MI0000717 | >gga-mir-147-2 | MI0003697 | >gga-mir-1780 | MI0007523 |
| >mmu-mir-19b-1 | MI0000718 | >gga-mir-193b | MI0003698 | >gga-mir-1781 | MI0007524 |
| >mmu-mir-92a-1 | MI0000719 | >gga-mir-202 | MI0003699 | >gga-mir-1782 | MI0007525 |
| >mmu-mir-9-1 | MI0000720 | >gga-mir-302b | MI0003700 | >gga-mir-1783 | MI0007526 |
| >mmu-mir-9-3 | MI0000721 | >gga-mir-302c | MI0003701 | >gga-mir-1784 | MI0007527 |
| >mmu-mir-138-1 | MI0000722 | >gga-mir-302d | MI0003702 | >gga-mir-1785 | MI0007528 |
| >mmu-mir-181b-1 | MI0000723 | >gga-mir-365-1 | MI0003703 | >gga-mir-1786 | MI0007529 |
| >mmu-mir-181c | MI0000724 | >gga-mir-365-2 | MI0003704 | >gga-mir-1787 | MI0007530 |
| >mmu-mir-125b-1 | MI0000725 | >gga-mir-375 | MI0003705 | >gga-mir-1788 | MI0007531 |
| >mmu-mir-128-2 | MI0000726 | >gga-mir-383 | MI0003706 | >gga-mir-1789 | MI0007532 |
| >hsa-mir-128-2 | MI0000727 | >gga-mir-455 | MI0003707 | >gga-mir-1790 | MI0007533 |
| >mmu-mir-7a-1 | MI0000728 | >gga-mir-489 | MI0003708 | >gga-mir-1791 | MI0007534 |
| >mmu-mir-7a-2 | MI0000729 | >gga-mir-490 | MI0003709 | >gga-mir-1792 | MI0007535 |
| >mmu-mir-7b | MI0000730 | >gga-mir-499 | MI0003710 | >gga-mir-1793 | MI0007536 |
| >mmu-mir-217 | MI0000731 | >gga-mir-211 | MI0003711 | >gga-mir-1794 | MI0007537 |
| >hsa-mir-194-2 | MI0000732 | >gga-mir-367 | MI0003712 | >gga-mir-1795 | MI0007538 |
| >mmu-mir-194-2 | MI0000733 | >gga-mir-466 | MI0003713 | >gga-mir-1796 | MI0007539 |
| >hsa-mir-106b | MI0000734 | >gga-mir-429 | MI0003714 | >gga-mir-1797 | MI0007540 |
| >hsa-mir-29c | MI0000735 | >gga-mir-449 | MI0003715 | >gga-mir-1756b | MI0007541 |
| >hsa-mir-30c-1 | MI0000736 | >mmu-mir-302b | MI0003716 | >gga-mir-1798 | MI0007542 |
| >hsa-mir-200a | MI0000737 | >mmu-mir-302c | MI0003717 | >gga-mir-1799 | MI0007543 |
| >hsa-mir-302a | MI0000738 | >mmu-mir-302d | MI0003718 | >gga-mir-1800 | MI0007544 |
| >hsa-mir-101-2 | MI0000739 | >rno-mir-378 | MI0003719 | >gga-mir-1801 | MI0007545 |
| >hsa-mir-219-2 | MI0000740 | >rno-mir-505 | MI0003720 | >gga-mir-1802 | MI0007546 |
| >mmu-mir-219-2 | MI0000741 | >rno-mir-499 | MI0003721 | >gga-mir-122b | MI0007547 |
| >hsa-mir-34b | MI0000742 | >rno-mir-664-1 | MI0003722 | >gga-mir-1803 | MI0007548 |
| >hsa-mir-34c | MI0000743 | >rno-mir-664-2 | MI0003723 | >gga-mir-1804 | MI0007549 |
| >hsa-mir-299 | MI0000744 | >rno-mir-497 | MI0003724 | >gga-mir-1805 | MI0007550 |
| >hsa-mir-301a | MI0000745 | >ebv-mir-BART3 | MI0003725 | >gga-mir-1806 | MI0007551 |
| >hsa-mir-99b | MI0000746 | >ebv-mir-BART4 | MI0003726 | >gga-mir-1807 | MI0007552 |
| >hsa-mir-296 | MI0000747 | >ebv-mir-BART5 | MI0003727 | >gga-mir-1808 | MI0007553 |
| >hsa-mir-130b | MI0000748 | >ebv-mir-BART6 | MI0003728 | >gga-mir-1809 | MI0007554 |
| >hsa-mir-30e | MI0000749 | >ebv-mir-BART7 | MI0003729 | >gga-mir-1810 | MI0007555 |
| >hsa-mir-26a-2 | MI0000750 | >ebv-mir-BART8 | MI0003730 | >gga-mir-1811 | MI0007556 |
| >cbr-mir-72 | MI0000751 | >ebv-mir-BART9 | MI0003731 | >gga-mir-1812 | MI0007557 |
| >cel-mir-353 | MI0000752 | >ebv-mir-BART10 | MI0003732 | >gga-mir-101-2 | MI0007558 |
| >cel-mir-354 | MI0000753 | >ebv-mir-BART11 | MI0003733 | >gga-mir-10a | MI0007559 |
| >cel-mir-355 | MI0000754 | >ebv-mir-BART12 | MI0003734 | >gga-mir-130c | MI0007560 |
| >cel-mir-356 | MI0000755 | >ebv-mir-BART13 | MI0003735 | >gga-mir-146c | MI0007561 |
| >cel-mir-357 | MI0000756 | >ebv-mir-BART14 | MI0003736 | >gga-mir-15c | MI0007562 |
| >cel-mir-358 | MI0000757 | >rlcv-mir-rL1-1 | MI0003737 | >gga-mir-16c | MI0007563 |
| >cel-mir-359 | MI0000758 | >rlcv-mir-rL1-2 | MI0003738 | >gga-mir-193a | MI0007564 |
| >cel-mir-360 | MI0000759 | >rlcv-mir-rL1-3 | MI0003739 | >gga-mir-301b | MI0007565 |
| >hsa-mir-361 | MI0000760 | >rlcv-mir-rL1-4 | MI0003740 | >gga-mir-449b | MI0007566 |
| >mmu-mir-361 | MI0000761 | >rlcv-mir-rL1-5 | MI0003741 | >gga-mir-458 | MI0007567 |
| >hsa-mir-362 | MI0000762 | >rlcv-mir-rL1-6 | MI0003742 | >mml-mir-1-1 | MI0007568 |
| >mmu-mir-362 | MI0000763 | >rlcv-mir-rL1-7 | MI0003743 | >mml-mir-1-2 | MI0007569 |
| >hsa-mir-363 | MI0000764 | >rlcv-mir-rL1-8 | MI0003744 | >mml-let-7a-1 | MI0007570 |
| >mmu-mir-363 | MI0000765 | >rlcv-mir-rL1-9 | MI0003745 | >mml-let-7a-2 | MI0007571 |
| >hsa-mir-365-1 | MI0000767 | >rlcv-mir-rL1-10 | MI0003746 | >mml-let-7a-3 | MI0007572 |
| >mmu-mir-365-1 | MI0000768 | >rlcv-mir-rL1-11 | MI0003747 | >mml-let-7b | MI0007573 |
| >hsa-mir-365-2 | MI0000769 | >rlcv-mir-rL1-12 | MI0003748 | >mml-let-7c | MI0007574 |
| >hsa-mir-302b | MI0000772 | >rlcv-mir-rL1-13 | MI0003749 | >mml-let-7d | MI0007575 |
| >hsa-mir-302c | MI0000773 | >rlcv-mir-rL1-14 | MI0003750 | >mml-let-7e | MI0007576 |
| >hsa-mir-302d | MI0000774 | >rlcv-mir-rL1-15 | MI0003751 | >mml-let-7f-1 | MI0007577 |
| >hsa-mir-367 | MI0000775 | >rlcv-mir-rL1-16 | MI0003752 | >mml-let-7f-2 | MI0007578 |
| >hsa-mir-376c | MI0000776 | >hsa-mir-758 | MI0003757 | >mml-let-7g | MI0007579 |
| >hsa-mir-369 | MI0000777 | >hsa-mir-1264 | MI0003758 | >mml-let-7i | MI0007580 |
| >hsa-mir-370 | MI0000778 | >hsa-mir-671 | MI0003760 | >mml-mir-7-1 | MI0007581 |
| >hsa-mir-371 | MI0000779 | >hsa-mir-668 | MI0003761 | >mml-mir-7-2 | MI0007582 |
| >hsa-mir-372 | MI0000780 | >hsa-mir-767 | MI0003763 | >mml-mir-7-3 | MI0007583 |
| >hsa-mir-373 | MI0000781 | >hsa-mir-1224 | MI0003764 | >mml-mir-9-1 | MI0007584 |
| >hsa-mir-374a | MI0000782 | >hsa-mir-320b-1 | MI0003776 | >mml-mir-9-2 | MI0007585 |
| >hsa-mir-375 | MI0000783 | >hsa-mir-320c-1 | MI0003778 | >mml-mir-9-3 | MI0007586 |
| >hsa-mir-376a-1 | MI0000784 | >hsa-mir-1296 | MI0003780 | >mml-mir-10a | MI0007587 |
| >hsa-mir-377 | MI0000785 | >hsa-mir-1468 | MI0003782 | >mml-mir-10b | MI0007588 |
| >hsa-mir-378 | MI0000786 | >hsa-mir-1323 | MI0003786 | >mml-mir-16-2 | MI0007589 |
| >hsa-mir-379 | MI0000787 | >hsa-mir-1271 | MI0003814 | >mml-mir-18b | MI0007590 |
| >hsa-mir-380 | MI0000788 | >hsa-mir-1301 | MI0003815 | >mml-mir-20b | MI0007591 |
| >hsa-mir-381 | MI0000789 | >hsa-mir-454 | MI0003820 | >mml-mir-23b | MI0007592 |
| >hsa-mir-382 | MI0000790 | >hsa-mir-1185-2 | MI0003821 | >mml-mir-26a-2 | MI0007593 |
| >hsa-mir-383 | MI0000791 | >hsa-mir-1283-1 | MI0003832 | >mml-mir-26b | MI0007594 |
| >mmu-mir-375 | MI0000792 | >hsa-mir-675b | MI0003833 | >mml-mir-27b | MI0007595 |
| >mmu-mir-376a | MI0000793 | >hsa-mir-769 | MI0003834 | >mml-mir-29b-1 | MI0007596 |
| >mmu-mir-377 | MI0000794 | >hsa-mir-766 | MI0003836 | >mml-mir-29b-2 | MI0007597 |
| >mmu-mir-378 | MI0000795 | >hsa-mir-320b-2 | MI0003839 | >mml-mir-29c | MI0007598 |
| >mmu-mir-379 | MI0000796 | >hsa-mir-1185-1 | MI0003844 | >mml-mir-30c-1 | MI0007599 |
| >mmu-mir-380 | MI0000797 | >hsa-mir-802 | MI0003906 | >mml-mir-30c-2 | MI0007600 |
| >mmu-mir-381 | MI0000798 | >hsa-mir-1298 | MI0003938 | >mml-mir-30d | MI0007601 |
| >mmu-mir-382 | MI0000799 | >mmu-mir-1224 | MI0004118 | >mml-mir-30e | MI0007602 |
| >mmu-mir-383 | MI0000800 | >mmu-mir-301b | MI0004122 | >mml-mir-33b | MI0007603 |
| >cel-lsy-6 | MI0000801 | >mmu-mir-675 | MI0004123 | >mml-mir-34b | MI0007604 |
| >hsa-mir-340 | MI0000802 | >mmu-mir-744 | MI0004124 | >mml-mir-34c | MI0007605 |
| >hsa-mir-330 | MI0000803 | >mmu-mir-374 | MI0004125 | >mml-mir-92a-2 | MI0007606 |
| >hsa-mir-328 | MI0000804 | >mmu-mir-216b | MI0004126 | >mml-mir-92b | MI0007607 |
| >hsa-mir-342 | MI0000805 | >mmu-mir-592 | MI0004127 | >mml-mir-95 | MI0007608 |
| >hsa-mir-337 | MI0000806 | >mmu-mir-758 | MI0004129 | >mml-mir-99b | MI0007609 |
| >hsa-mir-323 | MI0000807 | >mmu-mir-551b | MI0004131 | >mml-mir-101-2 | MI0007610 |
| >hsa-mir-326 | MI0000808 | >mmu-mir-671 | MI0004133 | >mml-mir-103-2 | MI0007611 |
| >hsa-mir-151 | MI0000809 | >mmu-mir-668 | MI0004134 | >mml-mir-105-2 | MI0007612 |
| >hsa-mir-135b | MI0000810 | >mmu-mir-665 | MI0004171 | >mml-mir-122a | MI0007613 |
| >hsa-mir-148b | MI0000811 | >mmu-mir-667 | MI0004196 | >mml-mir-124a-2 | MI0007614 |
| >hsa-mir-331 | MI0000812 | >mmu-mir-770 | MI0004203 | >mml-mir-125a | MI0007615 |
| >hsa-mir-324 | MI0000813 | >mmu-mir-762 | MI0004215 | >mml-mir-126 | MI0007616 |
| >hsa-mir-338 | MI0000814 | >mmu-mir-802 | MI0004249 | >mml-mir-128b | MI0007617 |
| >hsa-mir-339 | MI0000815 | >mmu-mir-672 | MI0004258 | >mml-mir-129 | MI0007618 |
| >hsa-mir-335 | MI0000816 | >mmu-mir-670 | MI0004295 | >mml-mir-130b | MI0007619 |
| >mmu-mir-335 | MI0000817 | >mmu-mir-761 | MI0004306 | >mml-mir-132 | MI0007620 |
| >cbr-lsy-6 | MI0000818 | >mmu-mir-764 | MI0004310 | >mml-mir-133c | MI0007621 |
| >cel-mir-392 | MI0000819 | >mmu-mir-763 | MI0004516 | >mml-mir-133b | MI0007622 |
| >mmu-mir-133a-2 | MI0000820 | >mmu-mir-669a-1 | MI0004523 | >mml-mir-134 | MI0007623 |
| >mmu-mir-133b | MI0000821 | >mmu-mir-666 | MI0004553 | >mml-mir-135a-1 | MI0007624 |
| >hsa-mir-133b | MI0000822 | >mmu-mir-759 | MI0004554 | >mml-mir-135b | MI0007625 |
| >mmu-mir-181b-2 | MI0000823 | >mmu-mir-496 | MI0004589 | >mml-mir-136 | MI0007626 |
| >hsa-mir-325 | MI0000824 | >mmu-mir-673 | MI0004601 | >mml-mir-137 | MI0007627 |
| >hsa-mir-345 | MI0000825 | >mmu-mir-760 | MI0004605 | >mml-mir-138 | MI0007628 |
| >hsa-mir-346 | MI0000826 | >mmu-mir-674 | MI0004611 | >mml-mir-139 | MI0007629 |
| >rno-let-7a-1 | MI0000827 | >mmu-mir-488 | MI0004633 | >mml-mir-140 | MI0007630 |
| >rno-let-7a-2 | MI0000828 | >mmu-mir-677 | MI0004634 | >mml-mir-142 | MI0007631 |
| >rno-let-7b | MI0000829 | >mmu-mir-678 | MI0004635 | >mml-mir-143 | MI0007632 |
| >rno-let-7c-1 | MI0000830 | >mmu-mir-497 | MI0004636 | >mml-mir-144 | MI0007633 |
| >rno-let-7c-2 | MI0000831 | >mmu-mir-423 | MI0004637 | >mml-mir-146a | MI0007634 |
| >rno-let-7e | MI0000832 | >mmu-mir-679 | MI0004638 | >mml-mir-146b | MI0007635 |
| >rno-let-7f-1 | MI0000833 | >mmu-mir-495 | MI0004639 | >mml-mir-147a | MI0007636 |
| >rno-let-7f-2 | MI0000834 | >mmu-mir-680-1 | MI0004640 | >mml-mir-147b | MI0007637 |
| >rno-let-7i | MI0000835 | >mmu-mir-680-2 | MI0004641 | >mml-mir-148a | MI0007638 |
| >rno-mir-7a-2 | MI0000836 | >mmu-mir-680-3 | MI0004642 | >mml-mir-148b | MI0007639 |
| >rno-mir-7b | MI0000837 | >mmu-mir-681 | MI0004643 | >mml-mir-149 | MI0007640 |
| >rno-mir-9-1 | MI0000838 | >mmu-mir-682 | MI0004644 | >mml-mir-150 | MI0007641 |
| >rno-mir-9-3 | MI0000839 | >mmu-mir-449c | MI0004645 | >mml-mir-151 | MI0007642 |
| >rno-mir-9-2 | MI0000840 | >mmu-mir-683 | MI0004646 | >mml-mir-152 | MI0007643 |
| >rno-mir-10a | MI0000841 | >mmu-mir-684-1 | MI0004647 | >mml-mir-154 | MI0007644 |
| >rno-mir-10b | MI0000842 | >mmu-mir-684-2 | MI0004648 | >mml-mir-155 | MI0007645 |
| >rno-mir-15b | MI0000843 | >mmu-mir-685 | MI0004649 | >mml-mir-181b-2 | MI0007646 |
| >rno-mir-16 | MI0000844 | >mmu-mir-686 | MI0004650 | >mml-mir-181d | MI0007647 |
| >rno-mir-17-1 | MI0000845 | >mmu-mir-719 | MI0004651 | >mml-mir-184 | MI0007648 |
| >rno-mir-18a | MI0000846 | >mmu-mir-687 | MI0004652 | >mml-mir-185 | MI0007649 |
| >rno-mir-19b-1 | MI0000847 | >mmu-mir-688 | MI0004653 | >mml-mir-186 | MI0007650 |
| >rno-mir-19b-2 | MI0000848 | >mmu-mir-689-1 | MI0004654 | >mml-mir-187 | MI0007651 |
| >rno-mir-19a | MI0000849 | >mmu-mir-689-2 | MI0004655 | >mml-mir-190b | MI0007652 |
| >rno-mir-21 | MI0000850 | >mmu-mir-690 | MI0004658 | >mml-mir-191 | MI0007653 |
| >rno-mir-22 | MI0000851 | >mmu-mir-691 | MI0004659 | >mml-mir-192 | MI0007654 |
| >rno-mir-23a | MI0000852 | >mmu-mir-692-1 | MI0004660 | >mml-mir-193a | MI0007655 |
| >rno-mir-23b | MI0000853 | >mmu-mir-692-2 | MI0004661 | >mml-mir-193b | MI0007656 |
| >rno-mir-24-1 | MI0000854 | >mmu-mir-693 | MI0004662 | >mml-mir-194-2 | MI0007657 |
| >rno-mir-24-2 | MI0000855 | >mmu-mir-694 | MI0004664 | >mml-mir-195 | MI0007658 |
| >rno-mir-25 | MI0000856 | >mmu-mir-146b | MI0004665 | >mml-mir-196a-2 | MI0007659 |
| >rno-mir-26a | MI0000857 | >mmu-mir-669b | MI0004666 | >mml-mir-196b | MI0007660 |
| >rno-mir-26b | MI0000858 | >mmu-mir-669a-2 | MI0004667 | >mml-mir-197 | MI0007661 |
| >rno-mir-27b | MI0000859 | >mmu-mir-669a-3 | MI0004668 | >mml-mir-199a-2 | MI0007662 |
| >rno-mir-27a | MI0000860 | >mmu-mir-467b | MI0004671 | >mml-mir-200a | MI0007663 |
| >rno-mir-28 | MI0000861 | >mmu-mir-669c | MI0004673 | >mml-mir-203 | MI0007664 |
| >rno-mir-29b-2 | MI0000862 | >mmu-mir-297b | MI0004674 | >mml-mir-204 | MI0007665 |
| >rno-mir-29a | MI0000863 | >mmu-mir-695 | MI0004675 | >mml-mir-205 | MI0007666 |
| >rno-mir-29b-1 | MI0000864 | >mmu-mir-499 | MI0004676 | >mml-mir-206 | MI0007667 |
| >rno-mir-29c | MI0000865 | >mmu-mir-696 | MI0004677 | >mml-mir-208a | MI0007668 |
| >rno-mir-30c-1 | MI0000866 | >mmu-mir-720 | MI0004678 | >mml-mir-208b | MI0007669 |
| >rno-mir-30e | MI0000867 | >mmu-mir-455 | MI0004679 | >mml-mir-210 | MI0007670 |
| >rno-mir-30b | MI0000868 | >mmu-mir-491 | MI0004680 | >mml-mir-212 | MI0007671 |
| >rno-mir-30d | MI0000869 | >mmu-mir-697 | MI0004681 | >mml-mir-216a | MI0007672 |
| >rno-mir-30a | MI0000870 | >mmu-mir-698 | MI0004682 | >mml-mir-216b | MI0007673 |
| >rno-mir-30c-2 | MI0000871 | >mmu-mir-699 | MI0004683 | >mml-mir-217 | MI0007674 |
| >rno-mir-31 | MI0000872 | >mmu-mir-700 | MI0004684 | >mml-mir-218-1 | MI0007675 |
| >rno-mir-32 | MI0000873 | >mmu-mir-701 | MI0004685 | >mml-mir-219-2 | MI0007676 |
| >rno-mir-33 | MI0000874 | >mmu-mir-702 | MI0004686 | >mml-mir-220b | MI0007677 |
| >rno-mir-34b | MI0000875 | >mmu-mir-703 | MI0004687 | >mml-mir-220c | MI0007678 |
| >rno-mir-34c | MI0000876 | >mmu-mir-704 | MI0004688 | >mml-mir-220d | MI0007679 |
| >rno-mir-34a | MI0000877 | >mmu-mir-705 | MI0004689 | >mml-mir-222 | MI0007680 |
| >rno-mir-92a-1 | MI0000878 | >mmu-mir-706 | MI0004690 | >mml-mir-296 | MI0007681 |
| >rno-mir-92a-2 | MI0000879 | >mmu-mir-707 | MI0004691 | >mml-mir-297 | MI0007682 |
| >rno-mir-93 | MI0000880 | >mmu-mir-708 | MI0004692 | >mml-mir-298 | MI0007683 |
| >rno-mir-96 | MI0000881 | >mmu-mir-709 | MI0004693 | >mml-mir-299 | MI0007684 |
| >rno-mir-98 | MI0000882 | >mmu-mir-710 | MI0004694 | >mml-mir-301a | MI0007685 |
| >rno-mir-99a | MI0000883 | >mmu-mir-711 | MI0004695 | >mml-mir-301b | MI0007686 |
| >rno-mir-99b | MI0000884 | >mmu-mir-712 | MI0004696 | >mml-mir-302a | MI0007687 |
| >rno-mir-100 | MI0000885 | >mmu-mir-713 | MI0004698 | >mml-mir-302b | MI0007688 |
| >rno-mir-101a | MI0000886 | >mmu-mir-714 | MI0004699 | >mml-mir-302c | MI0007689 |
| >rno-mir-103-2 | MI0000887 | >mmu-mir-715 | MI0004700 | >mml-mir-302d | MI0007690 |
| >rno-mir-103-1 | MI0000888 | >mmu-mir-500 | MI0004702 | >mml-mir-320 | MI0007691 |
| >rno-mir-106b | MI0000889 | >mmu-mir-501 | MI0004703 | >mml-mir-323 | MI0007692 |
| >rno-mir-107 | MI0000890 | >mmu-mir-717 | MI0004704 | >mml-mir-324 | MI0007693 |
| >rno-mir-122 | MI0000891 | >mmu-mir-450b | MI0004705 | >mml-mir-325 | MI0007694 |
| >rno-mir-124-3 | MI0000892 | >mmu-mir-505 | MI0004706 | >mml-mir-329-1 | MI0007695 |
| >rno-mir-124-1 | MI0000893 | >mmu-mir-718 | MI0004707 | >mml-mir-329-2 | MI0007696 |
| >rno-mir-124-2 | MI0000894 | >mmu-mir-721 | MI0004708 | >mml-mir-330 | MI0007697 |
| >rno-mir-125a | MI0000895 | >ppt-MIR1211 | MI0004710 | >mml-mir-331 | MI0007698 |
| >rno-mir-125b-1 | MI0000896 | >ppt-MIR1212 | MI0004711 | >mml-mir-335 | MI0007699 |
| >rno-mir-125b-2 | MI0000897 | >ppt-MIR1214 | MI0004713 | >mml-mir-337 | MI0007700 |
| >rno-mir-126 | MI0000898 | >ppt-MIR1215 | MI0004714 | >mml-mir-338 | MI0007701 |
| >rno-mir-127 | MI0000899 | >ppt-MIR1216 | MI0004715 | >mml-mir-339 | MI0007702 |
| >rno-mir-128-1 | MI0000900 | >ppt-MIR1217 | MI0004716 | >mml-mir-340 | MI0007703 |
| >rno-mir-128-2 | MI0000901 | >ppt-MIR1218 | MI0004717 | >mml-mir-342 | MI0007704 |
| >rno-mir-129-1 | MI0000902 | >ppt-MIR1219a | MI0004718 | >mml-mir-345 | MI0007705 |
| >rno-mir-130a | MI0000903 | >ppt-MIR1219b | MI0004719 | >mml-mir-346 | MI0007706 |
| >rno-mir-130b | MI0000904 | >ppt-MIR1219c | MI0004720 | >mml-mir-361 | MI0007707 |
| >rno-mir-132 | MI0000905 | >ppt-MIR1219d | MI0004721 | >mml-mir-362 | MI0007708 |
| >rno-mir-133a | MI0000906 | >ppt-MIR1220a | MI0004722 | >mml-mir-363 | MI0007709 |
| >rno-mir-134 | MI0000907 | >ppt-MIR1220b | MI0004723 | >mml-mir-365-1 | MI0007710 |
| >rno-mir-135a | MI0000908 | >ppt-MIR533b | MI0004724 | >mml-mir-365-2 | MI0007711 |
| >rno-mir-136 | MI0000909 | >ppt-MIR535d | MI0004725 | >mml-mir-367 | MI0007712 |
| >rno-mir-137 | MI0000910 | >ppt-MIR1221 | MI0004726 | >mml-mir-369 | MI0007713 |
| >rno-mir-138-2 | MI0000911 | >ppt-MIR1222a | MI0004727 | >mml-mir-370 | MI0007714 |
| >rno-mir-138-1 | MI0000912 | >ppt-MIR1223a | MI0004728 | >mml-mir-371 | MI0007715 |
| >rno-mir-139 | MI0000913 | >ppt-MIR390c | MI0004729 | >mml-mir-372 | MI0007716 |
| >rno-mir-141 | MI0000914 | >hsv1-mir-H1 | MI0004730 | >mml-mir-373 | MI0007717 |
| >rno-mir-142 | MI0000915 | >bta-mir-26a | MI0004731 | >mml-mir-374a | MI0007718 |
| >rno-mir-143 | MI0000916 | >bta-mir-18b | MI0004732 | >mml-mir-374b | MI0007719 |
| >rno-mir-144 | MI0000917 | >bta-mir-29a | MI0004733 | >mml-mir-375 | MI0007720 |
| >rno-mir-145 | MI0000918 | >bta-let-7f-2 | MI0004734 | >mml-mir-376a-1 | MI0007721 |
| >rno-mir-146a | MI0000919 | >bta-mir-101 | MI0004735 | >mml-mir-376a-2 | MI0007722 |
| >rno-mir-150 | MI0000920 | >bta-mir-103-1 | MI0004736 | >mml-mir-376b | MI0007723 |
| >rno-mir-152 | MI0000921 | >bta-mir-148a | MI0004737 | >mml-mir-376c | MI0007724 |
| >rno-mir-153 | MI0000922 | >bta-mir-151 | MI0004738 | >mml-mir-377 | MI0007725 |
| >rno-mir-154 | MI0000923 | >bta-mir-16b | MI0004739 | >mml-mir-378 | MI0007726 |
| >rno-mir-181c | MI0000924 | >bta-mir-18a | MI0004740 | >mml-mir-379 | MI0007727 |
| >rno-mir-181a-2 | MI0000925 | >bta-mir-20a | MI0004741 | >mml-mir-380 | MI0007728 |
| >rno-mir-181b-1 | MI0000926 | >bta-mir-21 | MI0004742 | >mml-mir-381 | MI0007729 |
| >rno-mir-181b-2 | MI0000927 | >bta-mir-221 | MI0004743 | >mml-mir-382 | MI0007730 |
| >rno-mir-183 | MI0000928 | >bta-mir-222 | MI0004744 | >mml-mir-383 | MI0007731 |
| >rno-mir-184 | MI0000929 | >bta-mir-26b | MI0004745 | >mml-mir-384 | MI0007732 |
| >rno-mir-185 | MI0000930 | >bta-mir-27a | MI0004746 | >mml-mir-409 | MI0007733 |
| >rno-mir-186 | MI0000931 | >bta-mir-30d | MI0004747 | >mml-mir-410 | MI0007734 |
| >rno-mir-187 | MI0000932 | >bta-mir-320 | MI0004748 | >mml-mir-411 | MI0007735 |
| >rno-mir-190 | MI0000933 | >bta-mir-484 | MI0004749 | >mml-mir-412 | MI0007736 |
| >rno-mir-191 | MI0000934 | >bta-mir-499 | MI0004750 | >mml-mir-421 | MI0007737 |
| >rno-mir-192 | MI0000935 | >bta-mir-99a | MI0004751 | >mml-mir-422a | MI0007738 |
| >rno-mir-193 | MI0000936 | >bta-mir-125a | MI0004752 | >mml-mir-423 | MI0007739 |
| >rno-mir-194-1 | MI0000937 | >bta-mir-125b-1 | MI0004753 | >mml-mir-424 | MI0007740 |
| >rno-mir-194-2 | MI0000938 | >bta-mir-126 | MI0004754 | >mml-mir-425 | MI0007741 |
| >rno-mir-195 | MI0000939 | >bta-mir-128-1 | MI0004755 | >mml-mir-429 | MI0007742 |
| >rno-mir-196a | MI0000940 | >bta-mir-145 | MI0004756 | >mml-mir-431 | MI0007743 |
| >rno-mir-199a | MI0000941 | >bta-mir-181a | MI0004757 | >mml-mir-432 | MI0007744 |
| >rno-mir-200c | MI0000942 | >bta-mir-199a | MI0004758 | >mml-mir-433 | MI0007745 |
| >rno-mir-200a | MI0000943 | >bta-mir-205 | MI0004759 | >mml-mir-448 | MI0007746 |
| >rno-mir-200b | MI0000944 | >bta-mir-27b | MI0004760 | >mml-mir-449a | MI0007747 |
| >rno-mir-203 | MI0000945 | >bta-mir-30b | MI0004761 | >mml-mir-449b | MI0007748 |
| >rno-mir-204 | MI0000946 | >bta-mir-31 | MI0004762 | >mml-mir-450a-1 | MI0007749 |
| >rno-mir-205 | MI0000947 | >bta-mir-34b | MI0004763 | >mml-mir-450a-2 | MI0007750 |
| >rno-mir-206 | MI0000948 | >dre-mir-190b | MI0004764 | >mml-mir-450b | MI0007751 |
| >rno-mir-208 | MI0000949 | >dre-mir-722 | MI0004765 | >mml-mir-451 | MI0007752 |
| >rno-mir-210 | MI0000950 | >dre-mir-499 | MI0004766 | >mml-mir-452 | MI0007753 |
| >rno-mir-211 | MI0000951 | >dre-mir-723 | MI0004767 | >mml-mir-453 | MI0007754 |
| >rno-mir-212 | MI0000952 | >dre-mir-724 | MI0004768 | >mml-mir-454 | MI0007755 |
| >rno-mir-181a-1 | MI0000953 | >dre-mir-725 | MI0004769 | >mml-mir-455 | MI0007756 |
| >rno-mir-214 | MI0000954 | >dre-mir-726 | MI0004770 | >mml-mir-484 | MI0007757 |
| >rno-mir-216a | MI0000955 | >dre-mir-727 | MI0004771 | >mml-mir-485 | MI0007758 |
| >rno-mir-217 | MI0000956 | >dre-mir-728 | MI0004772 | >mml-mir-486 | MI0007759 |
| >rno-mir-218-2 | MI0000957 | >dre-mir-729 | MI0004773 | >mml-mir-487a | MI0007760 |
| >rno-mir-218-1 | MI0000958 | >dre-mir-34c | MI0004774 | >mml-mir-487b | MI0007761 |
| >rno-mir-219-1 | MI0000959 | >dre-mir-730 | MI0004775 | >mml-mir-488 | MI0007762 |
| >rno-mir-219-2 | MI0000960 | >dre-mir-731 | MI0004776 | >mml-mir-489 | MI0007763 |
| >rno-mir-221 | MI0000961 | >dre-mir-732 | MI0004777 | >mml-mir-490 | MI0007764 |
| >rno-mir-222 | MI0000962 | >dre-mir-733 | MI0004778 | >mml-mir-491 | MI0007765 |
| >rno-mir-223 | MI0000963 | >dre-mir-15c | MI0004779 | >mml-mir-492 | MI0007766 |
| >rno-mir-290 | MI0000964 | >dre-mir-734 | MI0004780 | >mml-mir-493 | MI0007767 |
| >rno-mir-291a | MI0000965 | >dre-mir-735 | MI0004781 | >mml-mir-494 | MI0007768 |
| >rno-mir-292 | MI0000966 | >dre-mir-736 | MI0004782 | >mml-mir-495 | MI0007769 |
| >rno-mir-296 | MI0000967 | >dre-mir-737 | MI0004783 | >mml-mir-496 | MI0007770 |
| >rno-mir-297 | MI0000968 | >dre-mir-738 | MI0004784 | >mml-mir-497 | MI0007771 |
| >rno-mir-298 | MI0000969 | >dre-mir-739 | MI0004785 | >mml-mir-498 | MI0007772 |
| >rno-mir-299 | MI0000970 | >dre-mir-740 | MI0004786 | >mml-mir-499 | MI0007773 |
| >rno-mir-300 | MI0000971 | >xtr-let-7b | MI0004787 | >mml-mir-500 | MI0007774 |
| >rno-mir-320 | MI0000972 | >xtr-mir-1a-2 | MI0004788 | >mml-mir-501 | MI0007775 |
| >mmu-mir-215 | MI0000974 | >xtr-mir-1a-1 | MI0004789 | >mml-mir-502 | MI0007776 |
| >ath-MIR167d | MI0000975 | >xtr-mir-7-1 | MI0004790 | >mml-mir-503 | MI0007777 |
| >ath-MIR169b | MI0000976 | >xtr-mir-7-3 | MI0004791 | >mml-mir-504 | MI0007778 |
| >ath-MIR169c | MI0000977 | >xtr-mir-7-2 | MI0004792 | >mml-mir-505 | MI0007779 |
| >ath-MIR169d | MI0000978 | >xtr-mir-9a-1 | MI0004793 | >mml-mir-511-1 | MI0007780 |
| >ath-MIR169e | MI0000979 | >xtr-mir-9a-2 | MI0004794 | >mml-mir-511-2 | MI0007781 |
| >ath-MIR169f | MI0000980 | >xtr-mir-9b | MI0004795 | >mml-mir-512-1 | MI0007782 |
| >ath-MIR169g | MI0000981 | >xtr-mir-10a | MI0004796 | >mml-mir-512-2 | MI0007783 |
| >ath-MIR169h | MI0000982 | >xtr-mir-10b | MI0004797 | >mml-mir-514-2 | MI0007784 |
| >ath-MIR169i | MI0000983 | >xtr-mir-10c | MI0004798 | >mml-mir-516a-1 | MI0007785 |
| >ath-MIR169j | MI0000984 | >xtr-mir-15a | MI0004799 | >mml-mir-516a-2 | MI0007786 |
| >ath-MIR169k | MI0000985 | >xtr-mir-15b | MI0004800 | >mml-mir-517a | MI0007787 |
| >ath-MIR169l | MI0000986 | >xtr-mir-16c | MI0004801 | >mml-mir-517b | MI0007788 |
| >ath-MIR169m | MI0000987 | >xtr-mir-16a | MI0004802 | >mml-mir-518a | MI0007789 |
| >ath-MIR169n | MI0000988 | >xtr-mir-17 | MI0004803 | >mml-mir-518b | MI0007790 |
| >ath-MIR171b | MI0000989 | >xtr-mir-19a | MI0004804 | >mml-mir-518c | MI0007791 |
| >ath-MIR171c | MI0000990 | >xtr-mir-19b-2 | MI0004805 | >mml-mir-518d | MI0007792 |
| >ath-MIR172c | MI0000991 | >xtr-mir-23a-2 | MI0004806 | >mml-mir-518e | MI0007793 |
| >ath-MIR172d | MI0000992 | >xtr-mir-26-1 | MI0004807 | >mml-mir-518f | MI0007794 |
| >ath-MIR390a | MI0001000 | >xtr-mir-26-2 | MI0004808 | >mml-mir-519a | MI0007795 |
| >ath-MIR390b | MI0001001 | >xtr-mir-27a | MI0004809 | >mml-mir-519b | MI0007796 |
| >ath-MIR391 | MI0001002 | >xtr-mir-27b | MI0004810 | >mml-mir-519c | MI0007797 |
| >ath-MIR393a | MI0001003 | >xtr-mir-29d | MI0004811 | >mml-mir-519d | MI0007798 |
| >ath-MIR393b | MI0001004 | >xtr-mir-29b | MI0004812 | >mml-mir-520a | MI0007799 |
| >ath-MIR394a | MI0001005 | >xtr-mir-30a | MI0004813 | >mml-mir-520b | MI0007800 |
| >ath-MIR394b | MI0001006 | >xtr-mir-30b | MI0004814 | >mml-mir-520c | MI0007801 |
| >ath-MIR395a | MI0001007 | >xtr-mir-30c-1 | MI0004815 | >mml-mir-520d | MI0007802 |
| >ath-MIR395b | MI0001008 | >xtr-mir-34a | MI0004816 | >mml-mir-520e | MI0007803 |
| >ath-MIR395c | MI0001009 | >xtr-mir-34b-2 | MI0004817 | >mml-mir-520f | MI0007804 |
| >ath-MIR395d | MI0001010 | >xtr-mir-34b-4 | MI0004818 | >mml-mir-520g | MI0007805 |
| >ath-MIR395e | MI0001011 | >xtr-mir-92a-2 | MI0004819 | >mml-mir-520h | MI0007806 |
| >ath-MIR395f | MI0001012 | >xtr-mir-98 | MI0004820 | >mml-mir-521 | MI0007807 |
| >ath-MIR396a | MI0001013 | >xtr-mir-99 | MI0004821 | >mml-mir-522 | MI0007808 |
| >ath-MIR396b | MI0001014 | >xtr-mir-106 | MI0004822 | >mml-mir-523a | MI0007809 |
| >ath-MIR397a | MI0001015 | >xtr-mir-107 | MI0004823 | >mml-mir-523b | MI0007810 |
| >ath-MIR397b | MI0001016 | >xtr-mir-122 | MI0004824 | >mml-mir-523c-1 | MI0007811 |
| >ath-MIR398a | MI0001017 | >xtr-mir-125b-1 | MI0004825 | >mml-mir-523c-2 | MI0007812 |
| >ath-MIR398b | MI0001018 | >xtr-mir-125b-2 | MI0004826 | >mml-mir-525 | MI0007813 |
| >ath-MIR398c | MI0001019 | >xtr-mir-126 | MI0004827 | >mml-mir-532 | MI0007814 |
| >ath-MIR399a | MI0001020 | >xtr-mir-128-2 | MI0004828 | >mml-mir-539 | MI0007815 |
| >ath-MIR399b | MI0001021 | >xtr-mir-129-1 | MI0004829 | >mml-mir-542 | MI0007816 |
| >ath-MIR399c | MI0001022 | >xtr-mir-129-2 | MI0004830 | >mml-mir-544 | MI0007817 |
| >ath-MIR399d | MI0001023 | >xtr-mir-130a | MI0004831 | >mml-mir-545 | MI0007818 |
| >ath-MIR399e | MI0001024 | >xtr-mir-130c | MI0004832 | >mml-mir-548a | MI0007819 |
| >ath-MIR399f | MI0001025 | >xtr-mir-130b | MI0004833 | >mml-mir-548b | MI0007820 |
| >osa-MIR393 | MI0001026 | >xtr-mir-132 | MI0004834 | >mml-mir-548c | MI0007821 |
| >osa-MIR394 | MI0001027 | >xtr-mir-133c | MI0004835 | >mml-mir-548d | MI0007822 |
| >osa-MIR395b | MI0001028 | >xtr-mir-133d | MI0004836 | >mml-mir-548e | MI0007823 |
| >osa-MIR395d | MI0001029 | >xtr-mir-133b | MI0004837 | >mml-mir-548f | MI0007824 |
| >osa-MIR395e | MI0001030 | >xtr-mir-135-1 | MI0004838 | >mml-mir-549 | MI0007825 |
| >osa-MIR395g | MI0001031 | >xtr-mir-138 | MI0004839 | >mml-mir-550 | MI0007826 |
| >osa-MIR395h | MI0001032 | >xtr-mir-139 | MI0004840 | >mml-mir-551a | MI0007827 |
| >osa-MIR395i | MI0001033 | >xtr-mir-140 | MI0004841 | >mml-mir-551b | MI0007828 |
| >osa-MIR395j | MI0001034 | >xtr-mir-142-1 | MI0004842 | >mml-mir-552 | MI0007829 |
| >osa-MIR395k | MI0001035 | >xtr-mir-142-2 | MI0004843 | >mml-mir-553 | MI0007830 |
| >osa-MIR395l | MI0001036 | >xtr-mir-146 | MI0004844 | >mml-mir-554 | MI0007831 |
| >osa-MIR395s | MI0001037 | >xtr-mir-148b | MI0004845 | >mml-mir-556 | MI0007832 |
| >osa-MIR395t | MI0001038 | >xtr-mir-150 | MI0004846 | >mml-mir-557 | MI0007833 |
| >osa-MIR395c | MI0001041 | >xtr-mir-153-1 | MI0004847 | >mml-mir-558 | MI0007834 |
| >osa-MIR395a | MI0001042 | >xtr-mir-155 | MI0004848 | >mml-mir-562 | MI0007835 |
| >osa-MIR395f | MI0001043 | >xtr-mir-181b-1 | MI0004849 | >mml-mir-563 | MI0007836 |
| >osa-MIR395u | MI0001044 | >xtr-mir-181b-2 | MI0004850 | >mml-mir-567 | MI0007837 |
| >osa-MIR396a | MI0001046 | >xtr-mir-182 | MI0004851 | >mml-mir-568 | MI0007838 |
| >osa-MIR396b | MI0001047 | >xtr-mir-183 | MI0004852 | >mml-mir-569 | MI0007839 |
| >osa-MIR396c | MI0001048 | >xtr-mir-184 | MI0004853 | >mml-mir-570 | MI0007840 |
| >osa-MIR397a | MI0001049 | >xtr-mir-187 | MI0004854 | >mml-mir-572 | MI0007841 |
| >osa-MIR397b | MI0001050 | >xtr-mir-192 | MI0004855 | >mml-mir-573 | MI0007842 |
| >osa-MIR398a | MI0001051 | >xtr-mir-193 | MI0004856 | >mml-mir-576 | MI0007843 |
| >osa-MIR398b | MI0001052 | >xtr-mir-194-1 | MI0004857 | >mml-mir-577 | MI0007844 |
| >osa-MIR399a | MI0001053 | >xtr-mir-194-2 | MI0004858 | >mml-mir-578 | MI0007845 |
| >osa-MIR399b | MI0001054 | >xtr-mir-199a | MI0004859 | >mml-mir-579 | MI0007846 |
| >osa-MIR399c | MI0001055 | >xtr-mir-202-1 | MI0004860 | >mml-mir-580 | MI0007847 |
| >osa-MIR399d | MI0001056 | >xtr-mir-202-2 | MI0004861 | >mml-mir-581 | MI0007848 |
| >osa-MIR399e | MI0001057 | >xtr-mir-205b | MI0004862 | >mml-mir-582 | MI0007849 |
| >osa-MIR399f | MI0001058 | >xtr-mir-206 | MI0004863 | >mml-mir-583 | MI0007850 |
| >osa-MIR399g | MI0001059 | >xtr-mir-210 | MI0004864 | >mml-mir-584 | MI0007851 |
| >osa-MIR399h | MI0001060 | >xtr-mir-181a-1 | MI0004865 | >mml-mir-586 | MI0007852 |
| >osa-MIR399i | MI0001061 | >xtr-mir-181a-2 | MI0004866 | >mml-mir-587 | MI0007853 |
| >osa-MIR399j | MI0001062 | >xtr-mir-214 | MI0004867 | >mml-mir-589 | MI0007854 |
| >osa-MIR399k | MI0001063 | >xtr-mir-215 | MI0004868 | >mml-mir-590 | MI0007855 |
| >ebv-mir-BHRF1-1 | MI0001064 | >xtr-mir-216 | MI0004869 | >mml-mir-592 | MI0007856 |
| >ebv-mir-BHRF1-2 | MI0001065 | >xtr-mir-217 | MI0004870 | >mml-mir-593 | MI0007857 |
| >ebv-mir-BHRF1-3 | MI0001066 | >xtr-mir-218-1 | MI0004871 | >mml-mir-597 | MI0007858 |
| >ebv-mir-BART1 | MI0001067 | >xtr-mir-218-2 | MI0004872 | >mml-mir-598 | MI0007859 |
| >ebv-mir-BART2 | MI0001068 | >xtr-mir-219 | MI0004873 | >mml-mir-599 | MI0007860 |
| >ath-MIR400 | MI0001069 | >xtr-mir-222 | MI0004874 | >mml-mir-600 | MI0007861 |
| >ath-MIR401 | MI0001070 | >xtr-mir-223 | MI0004875 | >mml-mir-601 | MI0007862 |
| >ath-MIR402 | MI0001071 | >xtr-mir-301-1 | MI0004876 | >mml-mir-604 | MI0007863 |
| >ath-MIR403 | MI0001072 | >xtr-mir-301-2 | MI0004877 | >mml-mir-605 | MI0007864 |
| >ath-MIR404 | MI0001073 | >xtr-mir-302 | MI0004878 | >mml-mir-607 | MI0007865 |
| >ath-MIR405a | MI0001074 | >xtr-mir-365-1 | MI0004879 | >mml-mir-609 | MI0007866 |
| >ath-MIR405b | MI0001075 | >xtr-mir-367 | MI0004880 | >mml-mir-611 | MI0007867 |
| >ath-MIR405d | MI0001077 | >xtr-mir-383 | MI0004881 | >mml-mir-612 | MI0007868 |
| >ath-MIR406 | MI0001078 | >xtr-mir-425 | MI0004882 | >mml-mir-615 | MI0007869 |
| >ath-MIR407 | MI0001079 | >xtr-mir-455 | MI0004883 | >mml-mir-616 | MI0007870 |
| >ath-MIR408 | MI0001080 | >xtr-mir-489 | MI0004884 | >mml-mir-618 | MI0007871 |
| >ath-MIR156g | MI0001082 | >xtr-mir-499 | MI0004885 | >mml-mir-619 | MI0007872 |
| >ath-MIR156h | MI0001083 | >xtr-let-7c | MI0004886 | >mml-mir-624 | MI0007873 |
| >ath-MIR158b | MI0001084 | >xtr-let-7f | MI0004887 | >mml-mir-625 | MI0007874 |
| >ath-MIR159c | MI0001085 | >xtr-let-7g | MI0004888 | >mml-mir-626 | MI0007875 |
| >ath-MIR319c | MI0001086 | >xtr-let-7i | MI0004889 | >mml-mir-627 | MI0007876 |
| >ath-MIR164c | MI0001087 | >xtr-mir-1b | MI0004890 | >mml-mir-628 | MI0007877 |
| >ath-MIR167c | MI0001088 | >xtr-mir-9-3 | MI0004891 | >mml-mir-631 | MI0007878 |
| >ath-MIR172e | MI0001089 | >xtr-mir-15c | MI0004892 | >mml-mir-632 | MI0007879 |
| >osa-MIR156k | MI0001090 | >xtr-mir-18a | MI0004893 | >mml-mir-633 | MI0007880 |
| >osa-MIR156l | MI0001091 | >xtr-mir-23a-1 | MI0004894 | >mml-mir-636 | MI0007881 |
| >osa-MIR159a | MI0001092 | >xtr-mir-24a | MI0004895 | >mml-mir-638 | MI0007882 |
| >osa-MIR159b | MI0001093 | >xtr-mir-24b | MI0004896 | >mml-mir-639 | MI0007883 |
| >osa-MIR159c | MI0001094 | >xtr-mir-29a | MI0004897 | >mml-mir-640 | MI0007884 |
| >osa-MIR159d | MI0001095 | >xtr-mir-30d | MI0004898 | >mml-mir-642 | MI0007885 |
| >osa-MIR159e | MI0001096 | >xtr-mir-92b | MI0004899 | >mml-mir-643 | MI0007886 |
| >osa-MIR159f | MI0001097 | >xtr-mir-93a | MI0004900 | >mml-mir-644 | MI0007887 |
| >osa-MIR319a | MI0001098 | >xtr-mir-93b | MI0004901 | >mml-mir-648 | MI0007888 |
| >osa-MIR319b | MI0001099 | >xtr-mir-96 | MI0004902 | >mml-mir-649 | MI0007889 |
| >osa-MIR160e | MI0001100 | >xtr-mir-101a-2 | MI0004903 | >mml-mir-650a-1 | MI0007890 |
| >osa-MIR160f | MI0001101 | >xtr-mir-103-1 | MI0004904 | >mml-mir-650a-2 | MI0007891 |
| >osa-MIR162b | MI0001102 | >xtr-mir-148a | MI0004905 | >mml-mir-650b | MI0007892 |
| >osa-MIR164c | MI0001103 | >xtr-mir-338-1 | MI0004906 | >mml-mir-650c | MI0007893 |
| >osa-MIR164d | MI0001104 | >xtr-let-7e-1 | MI0004907 | >mml-mir-650d | MI0007894 |
| >osa-MIR164e | MI0001105 | >xtr-let-7a | MI0004908 | >mml-mir-651 | MI0007895 |
| >osa-MIR166j | MI0001106 | >xtr-let-7e-2 | MI0004909 | >mml-mir-652 | MI0007896 |
| >osa-MIR166k | MI0001107 | >xtr-mir-16b | MI0004910 | >mml-mir-653 | MI0007897 |
| >osa-MIR166l | MI0001108 | >xtr-mir-20a | MI0004911 | >mml-mir-654 | MI0007898 |
| >osa-MIR167d | MI0001109 | >xtr-mir-22 | MI0004912 | >mml-mir-656 | MI0007899 |
| >osa-MIR167e | MI0001110 | >xtr-mir-23b | MI0004913 | >mml-mir-657 | MI0007900 |
| >osa-MIR167f | MI0001111 | >xtr-mir-25-1 | MI0004914 | >mml-mir-660 | MI0007901 |
| >osa-MIR167g | MI0001112 | >xtr-mir-25-2 | MI0004915 | >mml-mir-661 | MI0007902 |
| >osa-MIR167h | MI0001113 | >xtr-mir-27c-1 | MI0004916 | >mml-mir-662 | MI0007903 |
| >osa-MIR167i | MI0001114 | >xtr-mir-27c-2 | MI0004917 | >mml-mir-663 | MI0007904 |
| >osa-MIR168a | MI0001115 | >xtr-mir-29c | MI0004918 | >mml-mir-664 | MI0007905 |
| >osa-MIR168b | MI0001116 | >xtr-mir-30c-2 | MI0004919 | >mml-mir-668 | MI0007906 |
| >osa-MIR169b | MI0001117 | >xtr-mir-30e | MI0004920 | >mml-mir-671 | MI0007907 |
| >osa-MIR169c | MI0001118 | >xtr-mir-31 | MI0004921 | >mml-mir-675 | MI0007908 |
| >osa-MIR169d | MI0001119 | >xtr-mir-33a | MI0004922 | >mml-mir-758 | MI0007909 |
| >osa-MIR169e | MI0001120 | >xtr-mir-33b | MI0004923 | >mml-mir-765 | MI0007910 |
| >osa-MIR169f | MI0001121 | >xtr-mir-34b-3 | MI0004924 | >mml-mir-767 | MI0007911 |
| >osa-MIR169g | MI0001122 | >xtr-mir-34b-1 | MI0004925 | >mml-mir-770 | MI0007913 |
| >osa-MIR169h | MI0001123 | >xtr-mir-92a-1 | MI0004926 | >mml-mir-802 | MI0007914 |
| >osa-MIR169i | MI0001124 | >xtr-mir-100 | MI0004927 | >mml-mir-874 | MI0007915 |
| >osa-MIR169j | MI0001125 | >xtr-mir-101a-1 | MI0004928 | >mml-mir-875 | MI0007916 |
| >osa-MIR169k | MI0001126 | >xtr-mir-103-2 | MI0004929 | >mml-mir-876 | MI0007917 |
| >osa-MIR169l | MI0001127 | >xtr-mir-124 | MI0004930 | >mml-mir-877 | MI0007918 |
| >osa-MIR169m | MI0001128 | >xtr-mir-125a | MI0004931 | >mml-mir-885 | MI0007919 |
| >osa-MIR169n | MI0001129 | >xtr-mir-128-1 | MI0004932 | >mml-mir-886 | MI0007920 |
| >osa-MIR169o | MI0001130 | >xtr-mir-135-2 | MI0004933 | >mml-mir-887 | MI0007921 |
| >osa-MIR169p | MI0001131 | >xtr-mir-137-1 | MI0004934 | >mml-mir-888 | MI0007922 |
| >osa-MIR169q | MI0001132 | >xtr-mir-137-2 | MI0004935 | >mml-mir-889 | MI0007923 |
| >osa-MIR171b | MI0001133 | >xtr-mir-137-3 | MI0004936 | >mml-mir-890 | MI0007924 |
| >osa-MIR171c | MI0001134 | >xtr-mir-143 | MI0004937 | >mml-mir-891 | MI0007925 |
| >osa-MIR171d | MI0001135 | >xtr-mir-144 | MI0004938 | >mml-mir-892 | MI0007926 |
| >osa-MIR171e | MI0001136 | >xtr-mir-145 | MI0004939 | >mml-mir-920 | MI0007927 |
| >osa-MIR171f | MI0001137 | >xtr-mir-153-2 | MI0004940 | >mml-mir-922 | MI0007928 |
| >osa-MIR171g | MI0001138 | >xtr-mir-191 | MI0004941 | >mml-mir-924 | MI0007929 |
| >osa-MIR172a | MI0001139 | >xtr-mir-196a | MI0004942 | >mml-mir-933 | MI0007930 |
| >osa-MIR172b | MI0001140 | >xtr-mir-196b | MI0004943 | >mml-mir-934 | MI0007931 |
| >osa-MIR172c | MI0001141 | >xtr-mir-199b | MI0004944 | >mml-mir-936 | MI0007932 |
| >osa-MIR166g | MI0001142 | >xtr-mir-200a | MI0004945 | >mml-mir-937 | MI0007933 |
| >osa-MIR166h | MI0001143 | >xtr-mir-200b | MI0004946 | >mml-mir-938 | MI0007934 |
| >osa-MIR166i | MI0001144 | >xtr-mir-203 | MI0004947 | >mml-mir-939 | MI0007935 |
| >hsa-mir-384 | MI0001145 | >xtr-mir-204-2 | MI0004948 | >mml-mir-940 | MI0007936 |
| >mmu-mir-384 | MI0001146 | >xtr-mir-204-1 | MI0004949 | >mml-mir-942 | MI0007937 |
| >osa-MIR171h | MI0001147 | >xtr-mir-205a | MI0004950 | >mml-mir-944 | MI0007938 |
| >osa-MIR393b | MI0001148 | >xtr-mir-208 | MI0004951 | >vvi-MIR156h | MI0007939 |
| >osa-MIR408 | MI0001149 | >xtr-mir-212 | MI0004952 | >vvi-MIR169b | MI0007940 |
| >hsa-mir-196b | MI0001150 | >xtr-mir-221 | MI0004953 | >vvi-MIR169h | MI0007941 |
| >mmu-mir-196b | MI0001151 | >xtr-mir-338-2 | MI0004954 | >vvi-MIR169i | MI0007942 |
| >rno-mir-196b | MI0001152 | >xtr-mir-363 | MI0004955 | >vvi-MIR169l | MI0007943 |
| >osa-MIR172d | MI0001154 | >xtr-mir-429 | MI0004956 | >vvi-MIR169n | MI0007944 |
| >osa-MIR171i | MI0001155 | >xtr-mir-449 | MI0004957 | >vvi-MIR169o | MI0007945 |
| >osa-MIR167j | MI0001156 | >xtr-mir-451 | MI0004958 | >vvi-MIR169q | MI0007946 |
| >osa-MIR166m | MI0001157 | >xtr-mir-18b | MI0004959 | >vvi-MIR169v | MI0007947 |
| >osa-MIR166n | MI0001158 | >xtr-mir-19b-1 | MI0004960 | >vvi-MIR169w | MI0007948 |
| >osa-MIR164f | MI0001159 | >xtr-mir-20b | MI0004961 | >vvi-MIR169x | MI0007949 |
| >mmu-mir-409 | MI0001160 | >xtr-mir-133a | MI0004962 | >vvi-MIR171g | MI0007950 |
| >mmu-mir-410 | MI0001161 | >xtr-mir-428 | MI0004963 | >vvi-MIR319e | MI0007951 |
| >mmu-mir-376b | MI0001162 | >mmu-mir-652 | MI0004965 | >vvi-MIR393a | MI0007952 |
| >mmu-mir-411 | MI0001163 | >tni-mir-10d | MI0004966 | >vvi-MIR394c | MI0007953 |
| >mmu-mir-412 | MI0001164 | >fru-mir-10d | MI0004967 | >vvi-MIR395n | MI0007954 |
| >mmu-mir-370 | MI0001165 | >bmo-let-7 | MI0004968 | >vvi-MIR396c | MI0007955 |
| >gga-mir-29a | MI0001166 | >bmo-mir-1 | MI0004969 | >vvi-MIR397a | MI0007956 |
| >gga-mir-29b-1 | MI0001167 | >bmo-mir-7 | MI0004970 | >vvi-MIR397b | MI0007957 |
| >gga-let-7i | MI0001168 | >bmo-mir-8 | MI0004971 | >vvi-MIR398b | MI0007958 |
| >gga-mir-135a-2 | MI0001169 | >bmo-mir-9 | MI0004972 | >vvi-MIR398c | MI0007959 |
| >gga-mir-33-1 | MI0001170 | >bmo-mir-10 | MI0004973 | >vvi-MIR399c | MI0007960 |
| >gga-let-7a-3 | MI0001171 | >bmo-mir-14 | MI0004974 | >vvi-MIR399d | MI0007961 |
| >gga-let-7b | MI0001172 | >bmo-mir-34 | MI0004975 | >vvi-MIR399f | MI0007962 |
| >gga-mir-99a | MI0001173 | >bmo-mir-124 | MI0004976 | >vvi-MIR399i | MI0007963 |
| >gga-let-7c | MI0001174 | >bmo-mir-263b | MI0004977 | >vvi-MIR403a | MI0007964 |
| >gga-mir-125b | MI0001175 | >bmo-mir-263a | MI0004978 | >vvi-MIR403b | MI0007965 |
| >gga-mir-155 | MI0001176 | >bmo-mir-275 | MI0004979 | >vvi-MIR403c | MI0007966 |
| >gga-mir-222 | MI0001177 | >bmo-mir-276 | MI0004980 | >vvi-MIR403d | MI0007967 |
| >gga-mir-221 | MI0001178 | >bmo-mir-277 | MI0004981 | >vvi-MIR403e | MI0007968 |
| >gga-mir-92 | MI0001179 | >bmo-mir-279 | MI0004982 | >vvi-MIR403f | MI0007969 |
| >gga-mir-19b | MI0001180 | >bmo-mir-282 | MI0004983 | >vvi-MIR477 | MI0007970 |
| >gga-mir-20a | MI0001181 | >bmo-mir-283 | MI0004984 | >vvi-MIR482 | MI0007971 |
| >gga-mir-19a | MI0001182 | >bmo-mir-305 | MI0004985 | >vvi-MIR828a | MI0007972 |
| >gga-mir-18a | MI0001183 | >bmo-mir-307 | MI0004986 | >vvi-MIR828b | MI0007973 |
| >gga-mir-17 | MI0001184 | >kshv-mir-K12-12 | MI0004987 | >vvi-MIR845a | MI0007974 |
| >gga-mir-16-1 | MI0001185 | >ebv-mir-BART15 | MI0004988 | >vvi-MIR845b | MI0007975 |
| >gga-mir-15a | MI0001186 | >ebv-mir-BART16 | MI0004989 | >vvi-MIR845c | MI0007976 |
| >gga-mir-26a | MI0001187 | >ebv-mir-BART17 | MI0004990 | >vvi-MIR845d | MI0007977 |
| >gga-mir-153 | MI0001188 | >ebv-mir-BART18 | MI0004991 | >vvi-MIR845e | MI0007978 |
| >gga-mir-148a | MI0001189 | >ebv-mir-BART19 | MI0004992 | >cel-mir-1817 | MI0007979 |
| >gga-mir-196-2 | MI0001190 | >ebv-mir-BART20 | MI0004993 | >cel-mir-1818 | MI0007980 |
| >gga-mir-138-1 | MI0001191 | >gga-mir-21 | MI0004994 | >cel-mir-1819 | MI0007981 |
| >gga-mir-128-2 | MI0001192 | >gga-mir-451 | MI0004995 | >cel-mir-1820 | MI0007982 |
| >gga-mir-187 | MI0001193 | >gga-mir-144 | MI0004996 | >cel-mir-1821 | MI0007983 |
| >gga-mir-32 | MI0001194 | >gga-mir-456 | MI0004997 | >cel-mir-1822 | MI0007984 |
| >gga-mir-133a-1 | MI0001195 | >gga-mir-460 | MI0004998 | >cel-mir-1823 | MI0007985 |
| >gga-mir-1a-2 | MI0001196 | >gga-mir-757-1 | MI0004999 | >cel-mir-1824 | MI0007986 |
| >gga-mir-124a | MI0001197 | >gga-mir-757-2 | MI0005000 | >cfa-mir-216 | MI0007987 |
| >gga-mir-30d | MI0001198 | >mmu-mir-490 | MI0005002 | >cfa-mir-33 | MI0007988 |
| >gga-mir-30b | MI0001199 | >mmu-mir-676 | MI0005003 | >cfa-let-7a | MI0007989 |
| >gga-mir-216 | MI0001200 | >mmu-mir-615 | MI0005004 | >cfa-mir-26a-2 | MI0007990 |
| >gga-mir-217 | MI0001201 | >bta-mir-106 | MI0005005 | >cfa-mir-1835 | MI0007991 |
| >gga-mir-194 | MI0001202 | >bta-mir-107 | MI0005006 | >cfa-mir-32 | MI0007992 |
| >gga-mir-215 | MI0001203 | >bta-mir-10a | MI0005007 | >cfa-mir-204 | MI0007993 |
| >gga-mir-30a | MI0001204 | >bta-mir-127 | MI0005008 | >cfa-mir-31 | MI0007994 |
| >gga-mir-30c-2 | MI0001205 | >bta-mir-139 | MI0005009 | >cfa-mir-101-2 | MI0007995 |
| >gga-mir-133b | MI0001206 | >bta-mir-140 | MI0005010 | >cfa-mir-371 | MI0007996 |
| >gga-mir-206 | MI0001207 | >bta-mir-142 | MI0005011 | >cfa-mir-491 | MI0007997 |
| >gga-mir-223 | MI0001208 | >bta-mir-15b | MI0005012 | >cfa-mir-150 | MI0007998 |
| >gga-mir-18b | MI0001209 | >bta-mir-181b | MI0005013 | >cfa-mir-455 | MI0007999 |
| >gga-mir-106 | MI0001210 | >bta-mir-193a | MI0005014 | >cfa-mir-30a | MI0008000 |
| >gga-mir-302a | MI0001211 | >bta-mir-20b | MI0005015 | >cfa-mir-30c-2 | MI0008001 |
| >gga-mir-218-1 | MI0001212 | >bta-mir-215 | MI0005016 | >cfa-mir-206 | MI0008002 |
| >gga-mir-103-2 | MI0001213 | >bta-mir-218 | MI0005017 | >cfa-mir-99b | MI0008003 |
| >gga-mir-203 | MI0001214 | >bta-mir-30e | MI0005018 | >cfa-let-7e | MI0008004 |
| >gga-mir-107 | MI0001215 | >bta-mir-345 | MI0005019 | >cfa-mir-125a | MI0008005 |
| >gga-mir-10b | MI0001216 | >bta-mir-369 | MI0005020 | >cfa-let-7f | MI0008006 |
| >gga-mir-128-1 | MI0001217 | >bta-mir-380 | MI0005021 | >cfa-mir-219-1 | MI0008007 |
| >gga-mir-181a-1 | MI0001218 | >bta-mir-487a | MI0005022 | >cfa-mir-23b | MI0008008 |
| >gga-mir-181b-1 | MI0001219 | >bta-mir-545 | MI0005023 | >cfa-mir-27b | MI0008009 |
| >gga-mir-199-2 | MI0001220 | >bta-mir-92 | MI0005024 | >cfa-mir-24-1 | MI0008010 |
| >gga-mir-137 | MI0001221 | >bta-mir-98 | MI0005025 | >cfa-mir-151 | MI0008011 |
| >gga-mir-16-2 | MI0001222 | >bta-let-7d | MI0005026 | >cfa-mir-30d | MI0008012 |
| >gga-mir-15b | MI0001223 | >bta-mir-124a | MI0005027 | >cfa-mir-30b | MI0008013 |
| >gga-mir-190 | MI0001224 | >bta-mir-132 | MI0005028 | >cfa-mir-1836 | MI0008014 |
| >gga-mir-204-2 | MI0001225 | >bta-mir-138 | MI0005029 | >cfa-mir-122 | MI0008015 |
| >gga-mir-7-2 | MI0001226 | >bta-mir-148b | MI0005030 | >cfa-mir-196b | MI0008016 |
| >gga-mir-184 | MI0001227 | >bta-mir-17 | MI0005031 | >cfa-mir-183 | MI0008017 |
| >gga-mir-138-2 | MI0001228 | >bta-mir-181c | MI0005032 | >cfa-mir-148a | MI0008018 |
| >gga-mir-140 | MI0001229 | >bta-mir-186 | MI0005033 | >cfa-mir-129-1 | MI0008019 |
| >gga-let-7g | MI0001230 | >bta-mir-191 | MI0005034 | >cfa-mir-335 | MI0008020 |
| >gga-mir-135a-1 | MI0001231 | >bta-mir-192 | MI0005035 | >cfa-mir-29b-1 | MI0008021 |
| >gga-let-7d | MI0001232 | >bta-mir-199b | MI0005036 | >cfa-mir-29a | MI0008022 |
| >gga-let-7f | MI0001233 | >bta-mir-200a | MI0005037 | >cfa-mir-30e | MI0008023 |
| >gga-let-7a-1 | MI0001234 | >bta-mir-200c | MI0005038 | >cfa-mir-30c-1 | MI0008024 |
| >gga-mir-146a | MI0001235 | >bta-mir-210 | MI0005039 | >cfa-mir-135 | MI0008025 |
| >gga-mir-103-1 | MI0001236 | >bta-mir-214 | MI0005040 | >cfa-mir-383 | MI0008026 |
| >gga-mir-218-2 | MI0001237 | >bta-mir-22 | MI0005041 | >cfa-mir-1837 | MI0008028 |
| >gga-mir-205b | MI0001238 | >bta-mir-23a | MI0005042 | >cfa-mir-130a | MI0008029 |
| >gga-mir-130b | MI0001239 | >bta-mir-29b | MI0005043 | >cfa-mir-129-2 | MI0008030 |
| >gga-mir-301 | MI0001240 | >bta-mir-29c | MI0005044 | >cfa-mir-192 | MI0008031 |
| >gga-mir-130a | MI0001241 | >bta-mir-361 | MI0005045 | >cfa-mir-128-1 | MI0008032 |
| >gga-mir-181b-2 | MI0001242 | >bta-mir-423 | MI0005046 | >cfa-mir-7-1 | MI0008033 |
| >gga-mir-181a-2 | MI0001243 | >bta-mir-425 | MI0005047 | >cfa-mir-181c | MI0008034 |
| >gga-mir-126 | MI0001244 | >bta-mir-450 | MI0005048 | >cfa-mir-181d | MI0008035 |
| >gga-mir-199-1 | MI0001245 | >bta-mir-455 | MI0005049 | >cfa-let-7g | MI0008036 |
| >gga-mir-219 | MI0001246 | >bta-mir-93 | MI0005050 | >cfa-mir-191 | MI0008037 |
| >gga-mir-1a-1 | MI0001247 | >bta-let-7g | MI0005051 | >cfa-mir-425 | MI0008038 |
| >gga-mir-133a-2 | MI0001248 | >bta-mir-10b | MI0005052 | >cfa-mir-23a | MI0008039 |
| >gga-mir-200a | MI0001249 | >bta-mir-24-2 | MI0005053 | >cfa-mir-27a | MI0008040 |
| >gga-mir-200b | MI0001250 | >bta-mir-30a | MI0005054 | >cfa-mir-24-2 | MI0008041 |
| >gga-mir-34a | MI0001251 | >bta-mir-200b | MI0005055 | >cfa-mir-199-1 | MI0008042 |
| >gga-mir-124b-1 | MI0001252 | >bta-mir-7 | MI0005056 | >cfa-mir-708 | MI0008043 |
| >gga-mir-124b-2 | MI0001253 | >bta-let-7a-1 | MI0005057 | >cfa-mir-1838 | MI0008044 |
| >gga-mir-1b | MI0001254 | >bta-mir-150 | MI0005058 | >cfa-mir-139 | MI0008045 |
| >gga-mir-133c | MI0001255 | >bta-mir-342 | MI0005059 | >cfa-mir-138b | MI0008047 |
| >gga-mir-30e | MI0001256 | >bta-mir-487b | MI0005060 | >cfa-mir-15a | MI0008048 |
| >gga-mir-30c-1 | MI0001257 | >bta-mir-532 | MI0005061 | >cfa-mir-16-1 | MI0008049 |
| >gga-mir-100 | MI0001258 | >bta-let-7f-1 | MI0005062 | >cfa-mir-17 | MI0008050 |
| >gga-let-7a-2 | MI0001259 | >bta-mir-122 | MI0005063 | >cfa-mir-19a | MI0008051 |
| >gga-mir-34b | MI0001260 | >bta-mir-30c | MI0005064 | >cfa-mir-20 | MI0008052 |
| >gga-mir-34c | MI0001261 | >bta-let-7i | MI0005065 | >cfa-mir-19b-1 | MI0008054 |
| >gga-let-7j | MI0001262 | >bta-mir-23b | MI0005066 | >cfa-mir-92a-1 | MI0008055 |
| >gga-let-7k | MI0001263 | >bta-mir-25 | MI0005067 | >cfa-mir-138a | MI0008056 |
| >gga-mir-135a-3 | MI0001264 | >bta-mir-34c | MI0005068 | >cfa-mir-128-2 | MI0008057 |
| >gga-mir-29c | MI0001265 | >bta-mir-363 | MI0005069 | >cfa-mir-26a-1 | MI0008058 |
| >gga-mir-29b-2 | MI0001266 | >mtr-MIR395c | MI0005070 | >cfa-mir-499 | MI0008059 |
| >gga-mir-205a | MI0001267 | >mtr-MIR395d | MI0005071 | >cfa-mir-1-1 | MI0008060 |
| >gga-mir-196-1 | MI0001268 | >mtr-MIR395e | MI0005072 | >cfa-mir-124-5 | MI0008061 |
| >gga-mir-7-3 | MI0001269 | >mtr-MIR395f | MI0005073 | >cfa-mir-124-1 | MI0008062 |
| >gga-mir-101 | MI0001270 | >mtr-MIR395g | MI0005074 | >cfa-mir-320 | MI0008063 |
| >gga-mir-204-1 | MI0001271 | >mtr-MIR395h | MI0005075 | >cfa-mir-130b | MI0008064 |
| >gga-mir-7-1 | MI0001272 | >mtr-MIR395i | MI0005076 | >cfa-mir-185 | MI0008065 |
| >gga-mir-23b | MI0001273 | >mtr-MIR395j | MI0005077 | >cfa-mir-1306 | MI0008066 |
| >gga-mir-27b | MI0001274 | >mtr-MIR395k | MI0005078 | >cfa-mir-196a | MI0008068 |
| >gga-mir-24 | MI0001275 | >mtr-MIR395l | MI0005079 | >cfa-mir-148b | MI0008069 |
| >gga-mir-31 | MI0001276 | >mtr-MIR395m | MI0005080 | >cfa-mir-200c | MI0008070 |
| >gga-mir-122-1 | MI0001277 | >mtr-MIR395n | MI0005081 | >cfa-mir-1307 | MI0008071 |
| >gga-mir-183 | MI0001278 | >mtr-MIR395o | MI0005082 | >cfa-mir-107 | MI0008072 |
| >gga-mir-7b | MI0001279 | >mtr-MIR395p | MI0005083 | >cfa-mir-146b | MI0008073 |
| >gga-mir-122-2 | MI0001280 | >osa-MIR395m | MI0005084 | >cfa-mir-124-2 | MI0008074 |
| >gga-mir-142 | MI0001281 | >osa-MIR395n | MI0005085 | >cfa-mir-99a-1 | MI0008075 |
| >gga-mir-196-3 | MI0001282 | >osa-MIR395o | MI0005086 | >cfa-let-7c | MI0008076 |
| >gga-mir-9-2 | MI0001283 | >osa-MIR395p | MI0005087 | >cfa-mir-125b-2 | MI0008077 |
| >gga-mir-218-3 | MI0001284 | >osa-MIR395q | MI0005088 | >cfa-mir-155 | MI0008078 |
| >dps-bantam | MI0001287 | >osa-MIR395v | MI0005090 | >cfa-mir-218-1 | MI0008079 |
| >dps-let-7 | MI0001288 | >osa-MIR395w | MI0005091 | >cfa-mir-574 | MI0008080 |
| >dps-mir-1 | MI0001289 | >osa-MIR395r | MI0005092 | >cfa-mir-9-2 | MI0008081 |
| >dps-mir-2a-1 | MI0001290 | >mdv1-mir-M1 | MI0005093 | >cfa-mir-28 | MI0008082 |
| >dps-mir-2a-2 | MI0001291 | >mdv1-mir-M2 | MI0005094 | >cfa-mir-15b | MI0008083 |
| >dps-mir-2b-1 | MI0001292 | >mdv1-mir-M3 | MI0005095 | >cfa-mir-16-2 | MI0008084 |
| >dps-mir-2b-2 | MI0001293 | >mdv1-mir-M4 | MI0005096 | >cfa-mir-7-2 | MI0008085 |
| >dps-mir-2c | MI0001294 | >mdv1-mir-M5 | MI0005097 | >cfa-mir-9-3 | MI0008086 |
| >dps-mir-3 | MI0001295 | >mdv1-mir-M6 | MI0005098 | >cfa-mir-1839 | MI0008087 |
| >dps-mir-4 | MI0001296 | >mdv1-mir-M7 | MI0005099 | >cfa-mir-26b | MI0008088 |
| >dps-mir-iab-4 | MI0001297 | >mdv1-mir-M8 | MI0005100 | >cfa-mir-1840 | MI0008089 |
| >dps-mir-5 | MI0001298 | >ath-MIR771 | MI0005101 | >cfa-mir-664 | MI0008090 |
| >dps-mir-6-1 | MI0001299 | >ath-MIR472 | MI0005102 | >cfa-mir-194 | MI0008091 |
| >dps-mir-6-3 | MI0001300 | >ath-MIR773 | MI0005103 | >cfa-mir-143 | MI0008092 |
| >dps-mir-6-2 | MI0001301 | >ath-MIR774 | MI0005104 | >cfa-mir-378 | MI0008093 |
| >dps-mir-7 | MI0001302 | >ath-MIR775 | MI0005105 | >cfa-mir-146a | MI0008094 |
| >dps-mir-8 | MI0001303 | >ath-MIR776 | MI0005106 | >cfa-mir-1271 | MI0008095 |
| >dps-mir-9a | MI0001304 | >ath-MIR777 | MI0005107 | >cfa-mir-1841 | MI0008096 |
| >dps-mir-9b | MI0001305 | >ath-MIR778 | MI0005108 | >cfa-mir-218-2 | MI0008097 |
| >dps-mir-9c | MI0001306 | >ath-MIR779 | MI0005109 | >cfa-mir-103 | MI0008098 |
| >dps-mir-10 | MI0001307 | >ath-MIR780 | MI0005110 | >cfa-mir-328 | MI0008099 |
| >dps-mir-11 | MI0001308 | >ath-MIR781 | MI0005111 | >cfa-mir-140 | MI0008100 |
| >dps-mir-12 | MI0001309 | >ath-MIR782 | MI0005112 | >cfa-mir-34a | MI0008101 |
| >dps-mir-13a | MI0001310 | >ath-MIR783 | MI0005113 | >cfa-mir-99a-2 | MI0008102 |
| >dps-mir-13b-1 | MI0001311 | >ptc-MIR171k | MI0005114 | >cfa-mir-125b-1 | MI0008103 |
| >dps-mir-13b-2 | MI0001312 | >ptc-MIR403c | MI0005115 | >cfa-mir-497 | MI0008104 |
| >dps-mir-14 | MI0001313 | >hsa-mir-765 | MI0005116 | >cfa-mir-195 | MI0008105 |
| >dps-mir-31a | MI0001314 | >hsa-mir-770 | MI0005118 | >cfa-mir-34c | MI0008106 |
| >dps-mir-31b | MI0001315 | >sme-bantam-a | MI0005119 | >cfa-mir-101-1 | MI0008107 |
| >dps-mir-33 | MI0001316 | >sme-bantam-b | MI0005120 | >cfa-mir-186 | MI0008108 |
| >dps-mir-34 | MI0001317 | >sme-bantam-c | MI0005121 | >cfa-mir-106b | MI0008109 |
| >dps-mir-79 | MI0001318 | >sme-let-7a | MI0005122 | >cfa-mir-93 | MI0008110 |
| >dps-mir-87 | MI0001319 | >sme-let-7b | MI0005123 | >cfa-mir-25 | MI0008111 |
| >dps-mir-92a | MI0001320 | >sme-let-7c | MI0005124 | >cfa-mir-197 | MI0008112 |
| >dps-mir-92b | MI0001321 | >sme-lin-4 | MI0005125 | >cfa-mir-193b | MI0008113 |
| >dps-mir-100 | MI0001322 | >sme-mir-1a | MI0005128 | >cfa-mir-590 | MI0008114 |
| >dps-mir-124 | MI0001323 | >sme-mir-1b | MI0005129 | >cfa-mir-1842 | MI0008116 |
| >dps-mir-125 | MI0001324 | >sme-mir-1c | MI0005130 | >cfa-mir-137 | MI0008117 |
| >dps-mir-133 | MI0001325 | >sme-mir-2a-1 | MI0005131 | >cfa-mir-1-2 | MI0008118 |
| >dps-mir-184 | MI0001326 | >sme-mir-2a-2 | MI0005132 | >cfa-mir-92b | MI0008119 |
| >dps-mir-210 | MI0001327 | >sme-mir-2b | MI0005133 | >cfa-mir-350 | MI0008120 |
| >dps-mir-219 | MI0001328 | >sme-mir-2c | MI0005134 | >cfa-mir-29b-2 | MI0008121 |
| >dps-mir-263a | MI0001329 | >sme-mir-2d | MI0005135 | >cfa-mir-29c | MI0008122 |
| >dps-mir-263b | MI0001330 | >sme-mir-7a | MI0005136 | >cfa-mir-1843 | MI0008123 |
| >dps-mir-274 | MI0001331 | >sme-mir-7b | MI0005137 | >cfa-mir-199-2 | MI0008124 |
| >dps-mir-275 | MI0001332 | >sme-mir-7c | MI0005138 | >cfa-mir-9-1 | MI0008125 |
| >dps-mir-276a | MI0001333 | >sme-mir-8 | MI0005139 | >cfa-mir-181a-1 | MI0008126 |
| >dps-mir-276b | MI0001334 | >sme-mir-10 | MI0005140 | >cfa-mir-181b-1 | MI0008127 |
| >dps-mir-277 | MI0001335 | >sme-mir-12 | MI0005141 | >cfa-mir-342 | MI0008128 |
| >dps-mir-278 | MI0001336 | >sme-mir-13 | MI0005142 | >cfa-mir-345 | MI0008129 |
| >dps-mir-279 | MI0001337 | >sme-mir-745 | MI0005143 | >cfa-mir-493 | MI0008130 |
| >dps-mir-280 | MI0001338 | >sme-mir-746 | MI0005144 | >cfa-mir-433 | MI0008131 |
| >dps-mir-281-1 | MI0001339 | >sme-mir-31a | MI0005145 | >cfa-mir-127 | MI0008132 |
| >dps-mir-281-2 | MI0001340 | >sme-mir-31b | MI0005146 | >cfa-mir-136 | MI0008133 |
| >dps-mir-282 | MI0001341 | >sme-mir-36 | MI0005147 | >cfa-mir-379 | MI0008134 |
| >dps-mir-283 | MI0001342 | >sme-mir-61 | MI0005148 | >cfa-mir-411 | MI0008135 |
| >dps-mir-284 | MI0001343 | >sme-mir-71a-1 | MI0005149 | >cfa-mir-380 | MI0008136 |
| >dps-mir-285 | MI0001344 | >sme-mir-71a-2 | MI0005150 | >cfa-mir-323 | MI0008137 |
| >dps-mir-286 | MI0001345 | >sme-mir-71b | MI0005151 | >cfa-mir-329 | MI0008138 |
| >dps-mir-287 | MI0001346 | >sme-mir-71c | MI0005152 | >cfa-mir-543 | MI0008139 |
| >dps-mir-288 | MI0001347 | >sme-mir-79 | MI0005153 | >cfa-mir-495 | MI0008140 |
| >dps-mir-289 | MI0001348 | >sme-mir-87a | MI0005154 | >cfa-mir-376-3 | MI0008141 |
| >dps-mir-304 | MI0001349 | >sme-mir-87b | MI0005155 | >cfa-mir-376-2 | MI0008142 |
| >dps-mir-305 | MI0001350 | >sme-mir-92 | MI0005156 | >cfa-mir-376-1 | MI0008143 |
| >dps-mir-306 | MI0001351 | >sme-mir-124a | MI0005157 | >cfa-mir-487 | MI0008144 |
| >dps-mir-307 | MI0001352 | >sme-mir-124b | MI0005158 | >cfa-mir-382 | MI0008145 |
| >dps-mir-308 | MI0001353 | >sme-mir-124c | MI0005159 | >cfa-mir-485 | MI0008146 |
| >dps-mir-309 | MI0001354 | >sme-mir-125a | MI0005160 | >cfa-mir-409 | MI0008147 |
| >dps-mir-314 | MI0001355 | >sme-mir-125b | MI0005161 | >cfa-mir-369 | MI0008148 |
| >dps-mir-315 | MI0001356 | >sme-mir-133 | MI0005162 | >cfa-mir-410 | MI0008149 |
| >dps-mir-316 | MI0001357 | >sme-mir-184 | MI0005163 | >cfa-mir-219-2 | MI0008150 |
| >dps-mir-317 | MI0001358 | >sme-mir-190a | MI0005164 | >cfa-mir-199-3 | MI0008151 |
| >dps-mir-318 | MI0001359 | >sme-mir-190b | MI0005165 | >cfa-mir-181a-2 | MI0008152 |
| >dre-mir-7b | MI0001360 | >sme-mir-747 | MI0005166 | >cfa-mir-181b-2 | MI0008153 |
| >dre-mir-7a-1 | MI0001361 | >sme-mir-219 | MI0005167 | >cfa-mir-126 | MI0008154 |
| >dre-mir-7a-2 | MI0001362 | >sme-mir-277a | MI0005168 | >cfa-mir-212 | MI0008155 |
| >dre-mir-10a | MI0001363 | >sme-mir-277b | MI0005169 | >cfa-mir-132 | MI0008156 |
| >dre-mir-10b-1 | MI0001364 | >sme-mir-277c | MI0005170 | >cfa-mir-22 | MI0008157 |
| >dre-mir-34 | MI0001365 | >sme-mir-278 | MI0005171 | >cfa-mir-144 | MI0008158 |
| >dre-mir-181b-1 | MI0001366 | >sme-mir-281 | MI0005172 | >cfa-mir-193a | MI0008159 |
| >dre-mir-181b-2 | MI0001367 | >sme-mir-67 | MI0005173 | >cfa-mir-142 | MI0008160 |
| >dre-mir-182 | MI0001368 | >sme-mir-748 | MI0005174 | >cfa-mir-10 | MI0008161 |
| >dre-mir-183 | MI0001369 | >sme-mir-749 | MI0005175 | >cfa-mir-152 | MI0008162 |
| >dre-mir-187 | MI0001370 | >sme-mir-750 | MI0005176 | >cfa-mir-338 | MI0008163 |
| >dre-mir-192 | MI0001371 | >sme-mir-751 | MI0005177 | >cfa-mir-1844 | MI0008164 |
| >dre-mir-196a-1 | MI0001372 | >sme-mir-752 | MI0005178 | >cfa-mir-21 | MI0008165 |
| >dre-mir-199-1 | MI0001373 | >sme-mir-753 | MI0005179 | >cfa-mir-423a | MI0008166 |
| >dre-mir-199-2 | MI0001374 | >sme-mir-754 | MI0005180 | >cfa-mir-652 | MI0008167 |
| >dre-mir-199-3 | MI0001375 | >sme-mir-755 | MI0005181 | >cfa-mir-224 | MI0008168 |
| >dre-mir-203a | MI0001376 | >sme-mir-756 | MI0005182 | >cfa-mir-424 | MI0008169 |
| >dre-mir-204-1 | MI0001377 | >sme-mir-277d | MI0005183 | >cfa-mir-503 | MI0008170 |
| >dre-mir-205 | MI0001378 | >cel-mir-784 | MI0005184 | >cfa-mir-542 | MI0008171 |
| >dre-mir-210 | MI0001379 | >cel-mir-785 | MI0005185 | >cfa-mir-450b | MI0008172 |
| >dre-mir-181a-1 | MI0001380 | >cel-mir-786 | MI0005186 | >cfa-mir-106a | MI0008173 |
| >dre-mir-214 | MI0001381 | >cel-mir-787 | MI0005187 | >cfa-mir-19b-2 | MI0008174 |
| >dre-mir-216a-1 | MI0001382 | >cel-mir-788 | MI0005188 | >cfa-mir-92a-2 | MI0008175 |
| >dre-mir-217-1 | MI0001383 | >cel-mir-789-1 | MI0005189 | >cfa-mir-363 | MI0008176 |
| >dre-mir-219-1 | MI0001384 | >cel-mir-789-2 | MI0005190 | >cfa-mir-361 | MI0008177 |
| >dre-mir-219-2 | MI0001385 | >cel-mir-790 | MI0005191 | >cfa-mir-384 | MI0008178 |
| >dre-mir-220 | MI0001386 | >cel-mir-791 | MI0005192 | >cfa-mir-374a | MI0008179 |
| >dre-mir-221 | MI0001387 | >cel-mir-792 | MI0005193 | >cfa-mir-374b | MI0008180 |
| >dre-mir-222 | MI0001388 | >cel-mir-793 | MI0005194 | >cfa-mir-421 | MI0008181 |
| >dre-mir-223 | MI0001389 | >cel-mir-794 | MI0005195 | >cfa-mir-98 | MI0008182 |
| >cbr-mir-353 | MI0001390 | >cel-mir-795 | MI0005196 | >cfa-mir-221 | MI0008183 |
| >cbr-mir-64 | MI0001392 | >cel-mir-796 | MI0005197 | >cfa-mir-532 | MI0008184 |
| >cbr-mir-231 | MI0001394 | >cel-mir-797 | MI0005198 | >cfa-mir-500 | MI0008185 |
| >cbr-mir-356 | MI0001395 | >cel-mir-798 | MI0005199 | >cfa-mir-660 | MI0008186 |
| >cbr-mir-83 | MI0001396 | >cel-mir-799 | MI0005200 | >cfa-mir-502 | MI0008187 |
| >cbr-mir-246 | MI0001397 | >cel-mir-800 | MI0005201 | >cfa-mir-676 | MI0008188 |
| >cbr-mir-51 | MI0001398 | >mmu-mir-804 | MI0005203 | >cfa-let-7j | MI0008189 |
| >cbr-mir-357 | MI0001400 | >mmu-mir-805 | MI0005204 | >hsa-mir-320d-1 | MI0008190 |
| >cbr-mir-253 | MI0001401 | >mmu-mir-741 | MI0005205 | >hsa-mir-320c-2 | MI0008191 |
| >cbr-mir-70 | MI0001402 | >mmu-mir-742 | MI0005206 | >hsa-mir-320d-2 | MI0008192 |
| >cbr-mir-358 | MI0001403 | >mmu-mir-743a | MI0005207 | >hsa-mir-1825 | MI0008193 |
| >cbr-mir-61 | MI0001404 | >osa-MIR807a | MI0005209 | >hsa-mir-1826 | MI0008194 |
| >cbr-mir-360 | MI0001405 | >osa-MIR806a | MI0005210 | >hsa-mir-1827 | MI0008195 |
| >cbr-mir-239a | MI0001406 | >osa-MIR806b | MI0005211 | >cel-mir-1828 | MI0008196 |
| >cbr-mir-249 | MI0001407 | >osa-MIR806c | MI0005212 | >cel-mir-1829a | MI0008197 |
| >cbr-mir-240 | MI0001408 | >osa-MIR806d | MI0005213 | >cel-mir-1829b | MI0008198 |
| >cbr-mir-254 | MI0001409 | >osa-MIR806e | MI0005214 | >cel-mir-1829c | MI0008199 |
| >cbr-mir-239b | MI0001410 | >osa-MIR806f | MI0005215 | >cel-mir-1830 | MI0008200 |
| >cbr-mir-62 | MI0001411 | >osa-MIR806g | MI0005216 | >cel-mir-1831 | MI0008201 |
| >cbr-mir-55 | MI0001412 | >osa-MIR806h | MI0005217 | >cel-mir-1832 | MI0008202 |
| >cbr-mir-84 | MI0001413 | >osa-MIR807b | MI0005218 | >cel-mir-1833 | MI0008203 |
| >cbr-mir-354 | MI0001414 | >osa-MIR807c | MI0005219 | >cel-mir-1834 | MI0008204 |
| >cbr-mir-35 | MI0001415 | >osa-MIR808 | MI0005220 | >gga-mir-216b | MI0008205 |
| >cbr-mir-36 | MI0001416 | >osa-MIR809a | MI0005221 | >gga-mir-1845 | MI0008206 |
| >cbr-mir-38 | MI0001418 | >osa-MIR809b | MI0005222 | >gga-mir-739 | MI0008207 |
| >cbr-mir-39 | MI0001419 | >osa-MIR809c | MI0005223 | >gga-mir-214 | MI0008208 |
| >cbr-mir-40 | MI0001420 | >osa-MIR809d | MI0005224 | >gga-mir-762 | MI0008209 |
| >cbr-mir-41 | MI0001421 | >osa-MIR809e | MI0005225 | >gga-mir-124a-2 | MI0008210 |
| >cbr-mir-355 | MI0001422 | >osa-MIR809f | MI0005226 | >ssc-mir-15a | MI0008211 |
| >rno-mir-421 | MI0001423 | >osa-MIR809g | MI0005227 | >ssc-mir-16-1 | MI0008212 |
| >ath-MIR413 | MI0001424 | >osa-MIR809h | MI0005228 | >ssc-mir-16-2 | MI0008213 |
| >ath-MIR414 | MI0001425 | >osa-MIR810a | MI0005229 | >ssc-mir-17 | MI0008214 |
| >ath-MIR415 | MI0001426 | >osa-MIR811a | MI0005230 | >ssc-mir-30b | MI0008215 |
| >ath-MIR416 | MI0001427 | >osa-MIR811b | MI0005231 | >ssc-mir-34a | MI0008216 |
| >ath-MIR417 | MI0001428 | >osa-MIR811c | MI0005232 | >ssc-mir-130a | MI0008217 |
| >ath-MIR418 | MI0001429 | >osa-MIR812a | MI0005233 | >ssc-mir-185 | MI0008218 |
| >ath-MIR419 | MI0001430 | >osa-MIR812b | MI0005234 | >ssc-mir-199b | MI0008219 |
| >ath-MIR420 | MI0001431 | >osa-MIR812c | MI0005235 | >ssc-mir-210 | MI0008220 |
| >osa-MIR413 | MI0001433 | >osa-MIR812d | MI0005236 | >ssc-mir-221 | MI0008221 |
| >osa-MIR414 | MI0001434 | >osa-MIR812e | MI0005237 | >osa-MIR531b | MI0008222 |
| >osa-MIR415 | MI0001435 | >osa-MIR813 | MI0005238 | >osa-MIR1846d | MI0008223 |
| >osa-MIR416 | MI0001436 | >osa-MIR814a | MI0005239 | >osa-MIR1847 | MI0008224 |
| >osa-MIR417 | MI0001437 | >osa-MIR814b | MI0005240 | >osa-MIR1848 | MI0008225 |
| >osa-MIR418 | MI0001438 | >osa-MIR814c | MI0005241 | >osa-MIR1849 | MI0008226 |
| >osa-MIR419 | MI0001439 | >osa-MIR815a | MI0005242 | >osa-MIR1850 | MI0008227 |
| >osa-MIR420 | MI0001440 | >osa-MIR815b | MI0005243 | >osa-MIR1851 | MI0008228 |
| >ath-MIR426 | MI0001441 | >osa-MIR815c | MI0005244 | >osa-MIR1852 | MI0008229 |
| >osa-MIR426 | MI0001442 | >osa-MIR816 | MI0005245 | >osa-MIR1853 | MI0008230 |
| >hsa-mir-422a | MI0001444 | >osa-MIR817 | MI0005246 | >osa-MIR1854 | MI0008231 |
| >hsa-mir-423 | MI0001445 | >osa-MIR818a | MI0005247 | >osa-MIR1855 | MI0008232 |
| >hsa-mir-424 | MI0001446 | >osa-MIR818b | MI0005248 | >osa-MIR1856 | MI0008233 |
| >mmu-mir-425 | MI0001447 | >osa-MIR818c | MI0005249 | >osa-MIR1857 | MI0008234 |
| >hsa-mir-425 | MI0001448 | >osa-MIR818d | MI0005250 | >osa-MIR1428b | MI0008235 |
| >xla-mir-427 | MI0001449 | >osa-MIR818e | MI0005251 | >osa-MIR1428c | MI0008237 |
| >xla-mir-428 | MI0001450 | >osa-MIR819a | MI0005252 | >osa-MIR1428d | MI0008238 |
| >xla-mir-429 | MI0001451 | >osa-MIR819b | MI0005253 | >osa-MIR1428e | MI0008239 |
| >xla-mir-19b | MI0001452 | >osa-MIR819c | MI0005254 | >osa-MIR1858a | MI0008240 |
| >xla-mir-20 | MI0001453 | >osa-MIR819d | MI0005255 | >osa-MIR1858b | MI0008241 |
| >xla-mir-18 | MI0001454 | >osa-MIR819e | MI0005256 | >osa-MIR1846a | MI0008242 |
| >xla-mir-133a | MI0001455 | >osa-MIR819f | MI0005257 | >osa-MIR1846b | MI0008243 |
| >zma-MIR156d | MI0001456 | >osa-MIR819g | MI0005258 | >osa-MIR1859 | MI0008244 |
| >zma-MIR156f | MI0001457 | >osa-MIR819h | MI0005259 | >osa-MIR1860 | MI0008245 |
| >zma-MIR156g | MI0001458 | >osa-MIR819i | MI0005260 | >osa-MIR1884a | MI0008246 |
| >zma-MIR156b | MI0001459 | >osa-MIR819j | MI0005261 | >osa-MIR1861a | MI0008247 |
| >zma-MIR156c | MI0001460 | >osa-MIR819k | MI0005262 | >osa-MIR1861b | MI0008248 |
| >zma-MIR156e | MI0001461 | >osa-MIR820a | MI0005263 | >osa-MIR1861c | MI0008249 |
| >zma-MIR156a | MI0001462 | >osa-MIR820b | MI0005264 | >osa-MIR1861d | MI0008250 |
| >zma-MIR156h | MI0001463 | >osa-MIR820c | MI0005265 | >osa-MIR1861e | MI0008251 |
| >zma-MIR156i | MI0001464 | >osa-MIR821a | MI0005266 | >osa-MIR1861f | MI0008252 |
| >zma-MIR160a | MI0001465 | >osa-MIR821b | MI0005267 | >osa-MIR1861g | MI0008253 |
| >zma-MIR160c | MI0001466 | >osa-MIR821c | MI0005268 | >osa-MIR1861h | MI0008254 |
| >zma-MIR160d | MI0001467 | >mdo-mir-1 | MI0005269 | >osa-MIR1861i | MI0008255 |
| >zma-MIR160b | MI0001468 | >mdo-mir-7 | MI0005270 | >osa-MIR1861j | MI0008256 |
| >zma-MIR164a | MI0001469 | >mdo-mir-9-2 | MI0005271 | >osa-MIR1861k | MI0008257 |
| >zma-MIR164d | MI0001470 | >mdo-mir-9-1 | MI0005272 | >osa-MIR1861l | MI0008258 |
| >zma-MIR164b | MI0001471 | >mdo-mir-10a | MI0005273 | >osa-MIR1861m | MI0008259 |
| >zma-MIR164c | MI0001472 | >mdo-mir-10b | MI0005274 | >osa-MIR1861n | MI0008260 |
| >zma-MIR169a | MI0001473 | >mdo-mir-21 | MI0005275 | >osa-MIR1862a | MI0008261 |
| >zma-MIR169b | MI0001474 | >mdo-mir-22 | MI0005276 | >osa-MIR1862b | MI0008262 |
| >zma-MIR167a | MI0001475 | >mdo-mir-30a | MI0005277 | >osa-MIR1862c | MI0008263 |
| >zma-MIR167b | MI0001476 | >mdo-mir-31 | MI0005278 | >osa-MIR1863 | MI0008264 |
| >zma-MIR167d | MI0001477 | >mdo-mir-32 | MI0005279 | >osa-MIR1864 | MI0008265 |
| >zma-MIR167c | MI0001478 | >mdo-mir-34a | MI0005280 | >osa-MIR1865 | MI0008266 |
| >zma-MIR160e | MI0001479 | >mdo-mir-100 | MI0005281 | >osa-MIR1866 | MI0008267 |
| >zma-MIR166a | MI0001480 | >mdo-mir-101-2 | MI0005282 | >osa-MIR1867 | MI0008268 |
| >zma-MIR162 | MI0001481 | >mdo-mir-101-1 | MI0005283 | >osa-MIR1423b | MI0008269 |
| >zma-MIR166h | MI0001482 | >mdo-mir-103-1 | MI0005284 | >osa-MIR1868 | MI0008270 |
| >zma-MIR166e | MI0001483 | >mdo-mir-103-2 | MI0005285 | >osa-MIR812f | MI0008271 |
| >zma-MIR166i | MI0001485 | >mdo-mir-107 | MI0005286 | >osa-MIR1869 | MI0008272 |
| >zma-MIR166f | MI0001486 | >mdo-mir-122 | MI0005287 | >osa-MIR1870 | MI0008273 |
| >zma-MIR166g | MI0001487 | >mdo-mir-124a-1 | MI0005288 | >osa-MIR1871 | MI0008274 |
| >zma-MIR166b | MI0001488 | >mdo-mir-124a-2 | MI0005289 | >osa-MIR1872 | MI0008275 |
| >zma-MIR166c | MI0001489 | >mdo-mir-124a-3 | MI0005290 | >osa-MIR1873 | MI0008276 |
| >zma-MIR166d | MI0001490 | >mdo-mir-125b-1 | MI0005291 | >osa-MIR1874 | MI0008277 |
| >zma-MIR171a | MI0001491 | >mdo-mir-125b-2 | MI0005292 | >osa-MIR1875 | MI0008278 |
| >zma-MIR171b | MI0001492 | >mdo-mir-128 | MI0005293 | >osa-MIR1862d | MI0008279 |
| >zma-MIR172a | MI0001493 | >mdo-mir-129 | MI0005294 | >osa-MIR1876 | MI0008280 |
| >zma-MIR172d | MI0001494 | >mdo-mir-130a | MI0005295 | >osa-MIR1884b | MI0008281 |
| >zma-MIR172b | MI0001495 | >mdo-mir-133a | MI0005296 | >osa-MIR1862e | MI0008282 |
| >zma-MIR172c | MI0001496 | >mdo-mir-135a | MI0005297 | >osa-MIR1877 | MI0008283 |
| >sbi-MIR166d | MI0001497 | >mdo-mir-135b | MI0005298 | >osa-MIR1878 | MI0008284 |
| >sbi-MIR166c | MI0001498 | >mdo-mir-137 | MI0005299 | >osa-MIR1879 | MI0008285 |
| >sbi-MIR166b | MI0001499 | >mdo-mir-138 | MI0005300 | >osa-MIR1880 | MI0008286 |
| >sbi-MIR166a | MI0001500 | >mdo-mir-142 | MI0005301 | >osa-MIR1881 | MI0008287 |
| >sbi-MIR172b | MI0001501 | >mdo-mir-143 | MI0005302 | >osa-MIR1882a | MI0008288 |
| >sbi-MIR172c | MI0001502 | >mdo-mir-144 | MI0005303 | >osa-MIR1882b | MI0008289 |
| >sbi-MIR172a | MI0001503 | >mdo-mir-451 | MI0005304 | >osa-MIR1882c | MI0008290 |
| >sbi-MIR156a | MI0001504 | >mdo-mir-145 | MI0005305 | >osa-MIR1882d | MI0008291 |
| >sbi-MIR156c | MI0001505 | >mdo-mir-152 | MI0005306 | >osa-MIR1882e | MI0008292 |
| >sbi-MIR156b | MI0001506 | >mdo-mir-182 | MI0005307 | >osa-MIR1882f | MI0008293 |
| >sbi-MIR160d | MI0001507 | >mdo-mir-184 | MI0005308 | >osa-MIR1882g | MI0008294 |
| >sbi-MIR160a | MI0001508 | >mdo-mir-187 | MI0005309 | >osa-MIR1882h | MI0008295 |
| >sbi-MIR160c | MI0001509 | >mdo-mir-181c | MI0005310 | >osa-MIR812g | MI0008296 |
| >sbi-MIR160b | MI0001510 | >mdo-mir-186 | MI0005311 | >osa-MIR812h | MI0008297 |
| >sbi-MIR160e | MI0001511 | >mdo-mir-193 | MI0005312 | >osa-MIR812i | MI0008298 |
| >sbi-MIR164 | MI0001512 | >mdo-mir-196b | MI0005313 | >osa-MIR812j | MI0008299 |
| >sbi-MIR167a | MI0001513 | >mdo-mir-199b | MI0005314 | >osa-MIR1883a | MI0008300 |
| >sbi-MIR167b | MI0001514 | >mdo-mir-203 | MI0005315 | >osa-MIR1883b | MI0008301 |
| >sbi-MIR169b | MI0001515 | >mdo-mir-204 | MI0005316 | >ath-MIR1886 | MI0008303 |
| >sbi-MIR169a | MI0001516 | >mdo-mir-206 | MI0005317 | >ath-MIR1887 | MI0008304 |
| >gga-mir-20b | MI0001517 | >mdo-mir-208 | MI0005318 | >ath-MIR1888 | MI0008305 |
| >hsa-mir-18b | MI0001518 | >mdo-mir-214 | MI0005319 | >aga-mir-11 | MI0008306 |
| >hsa-mir-20b | MI0001519 | >mdo-mir-216 | MI0005320 | >aga-mir-981 | MI0008307 |
| >oar-mir-431 | MI0001520 | >mdo-mir-217 | MI0005321 | >aga-mir-87 | MI0008308 |
| >oar-mir-127 | MI0001521 | >mdo-mir-218 | MI0005322 | >aga-mir-1889 | MI0008309 |
| >oar-mir-432 | MI0001522 | >mdo-mir-219 | MI0005323 | >aga-mir-375 | MI0008310 |
| >oar-mir-136 | MI0001523 | >mdo-mir-223 | MI0005324 | >aga-mir-1890 | MI0008311 |
| >mmu-mir-431 | MI0001524 | >mdo-mir-338 | MI0005325 | >aga-mir-1891 | MI0008312 |
| >mmu-mir-433 | MI0001525 | >mdo-mir-365 | MI0005326 | >mmu-mir-1902 | MI0008313 |
| >mmu-mir-434 | MI0001526 | >mdo-mir-375 | MI0005327 | >mmu-mir-1897 | MI0008314 |
| >dre-mir-430a-1 | MI0001527 | >mdo-mir-383 | MI0005328 | >mmu-mir-1905 | MI0008315 |
| >dre-mir-430b-1 | MI0001528 | >mdo-mir-449 | MI0005329 | >mmu-mir-1895 | MI0008316 |
| >dre-mir-430c-1 | MI0001529 | >mdo-let-7a-2 | MI0005330 | >mmu-mir-1903 | MI0008317 |
| >sbi-MIR393 | MI0001530 | >mdo-let-7g | MI0005331 | >mmu-mir-1899 | MI0008318 |
| >sbi-MIR394a | MI0001531 | >mdo-let-7i | MI0005332 | >mmu-mir-1900 | MI0008319 |
| >sbi-MIR394b | MI0001532 | >mdo-mir-15a | MI0005333 | >mmu-mir-1892 | MI0008320 |
| >sbi-MIR395b | MI0001533 | >mdo-mir-16 | MI0005334 | >mmu-mir-1906 | MI0008321 |
| >sbi-MIR395a | MI0001534 | >mdo-mir-183 | MI0005335 | >mmu-mir-1896 | MI0008322 |
| >sbi-MIR395c | MI0001535 | >mdo-mir-96 | MI0005336 | >mmu-mir-1904 | MI0008323 |
| >sbi-MIR395d | MI0001536 | >mdo-mir-212 | MI0005337 | >mmu-mir-1898 | MI0008324 |
| >sbi-MIR395e | MI0001537 | >mdo-mir-132 | MI0005338 | >mmu-mir-1907 | MI0008325 |
| >sbi-MIR396b | MI0001538 | >mdo-mir-200c | MI0005339 | >mmu-mir-1894 | MI0008326 |
| >sbi-MIR396a | MI0001539 | >mdo-mir-141 | MI0005340 | >mmu-mir-1893 | MI0008327 |
| >sbi-MIR396c | MI0001540 | >mdo-mir-191 | MI0005341 | >mmu-mir-1901 | MI0008328 |
| >sbi-MIR399a | MI0001541 | >mdo-mir-425 | MI0005342 | >hsa-mir-1908 | MI0008329 |
| >sbi-MIR399c | MI0001542 | >mdo-mir-181a | MI0005343 | >hsa-mir-1909 | MI0008330 |
| >sbi-MIR399d | MI0001543 | >mdo-mir-181b | MI0005344 | >hsa-mir-1910 | MI0008331 |
| >sbi-MIR399e | MI0001544 | >mdo-mir-200b | MI0005345 | >hsa-mir-1911 | MI0008332 |
| >sbi-MIR399f | MI0001545 | >mdo-mir-200a | MI0005346 | >hsa-mir-1912 | MI0008333 |
| >sbi-MIR399b | MI0001546 | >mdo-mir-222a | MI0005347 | >hsa-mir-1913 | MI0008334 |
| >sbi-MIR399g | MI0001547 | >mdo-mir-221 | MI0005348 | >hsa-mir-1914 | MI0008335 |
| >sbi-MIR156d | MI0001548 | >mdo-let-7f-2 | MI0005349 | >hsa-mir-1915 | MI0008336 |
| >sbi-MIR164b | MI0001549 | >mdo-let-7a-3 | MI0005350 | >mdv1-miR-M31 | MI0008337 |
| >sbi-MIR166e | MI0001550 | >mdo-let-7b | MI0005351 | >bmo-mir-2a-1 | MI0008338 |
| >sbi-MIR167d | MI0001551 | >mdo-mir-29b | MI0005352 | >bmo-mir-2a-2 | MI0008339 |
| >sbi-MIR167f | MI0001552 | >mdo-mir-29a | MI0005353 | >bmo-mir-2b | MI0008340 |
| >sbi-MIR167g | MI0001553 | >mdo-mir-17 | MI0005354 | >bmo-mir-13a | MI0008341 |
| >sbi-MIR167e | MI0001554 | >mdo-mir-18 | MI0005355 | >bmo-mir-13b | MI0008342 |
| >sbi-MIR167c | MI0001555 | >mdo-mir-19a | MI0005356 | >bmo-mir-79 | MI0008343 |
| >sbi-MIR168 | MI0001556 | >mdo-mir-20 | MI0005357 | >bmo-mir-87 | MI0008344 |
| >sbi-MIR169c | MI0001557 | >mdo-mir-19b | MI0005358 | >bmo-mir-133 | MI0008345 |
| >sbi-MIR169d | MI0001558 | >mdo-mir-92 | MI0005359 | >bmo-mir-184 | MI0008346 |
| >sbi-MIR169e | MI0001559 | >mdo-let-7a-1 | MI0005360 | >bmo-mir-210 | MI0008347 |
| >sbi-MIR169f | MI0001560 | >mdo-let-7f-1 | MI0005361 | >bmo-mir-281 | MI0008348 |
| >sbi-MIR169g | MI0001561 | >mdo-let-7d | MI0005362 | >bmo-mir-317 | MI0008349 |
| >sbi-MIR169h | MI0001562 | >mdo-mir-23a | MI0005363 | >bmo-bantam | MI0008350 |
| >sbi-MIR169i | MI0001563 | >mdo-mir-27a | MI0005364 | >sly-MIR1916 | MI0008351 |
| >sbi-MIR171b | MI0001564 | >mdo-mir-24-2 | MI0005365 | >sly-MIR1917 | MI0008352 |
| >sbi-MIR171d | MI0001565 | >mdo-mir-23b | MI0005366 | >sly-MIR1918 | MI0008353 |
| >sbi-MIR171a | MI0001566 | >mdo-mir-27b | MI0005367 | >sly-MIR1919a | MI0008354 |
| >sbi-MIR171c | MI0001567 | >mdo-mir-24-1 | MI0005368 | >sly-MIR1919b | MI0008355 |
| >sbi-MIR172e | MI0001568 | >mdo-mir-93 | MI0005369 | >sly-MIR1919c | MI0008356 |
| >sbi-MIR166f | MI0001569 | >mdo-mir-25 | MI0005370 | >sly-MIR160a | MI0008357 |
| >sbi-MIR171e | MI0001570 | >mdo-mir-302b | MI0005371 | >sly-MIR166a | MI0008358 |
| >sbi-MIR172d | MI0001571 | >mdo-mir-302c | MI0005372 | >sly-MIR166b | MI0008359 |
| >sbi-MIR159 | MI0001572 | >mdo-mir-302a | MI0005373 | >sly-MIR167 | MI0008360 |
| >sbi-MIR319 | MI0001573 | >mdo-mir-302d | MI0005374 | >sly-MIR169a | MI0008361 |
| >ame-bantam | MI0001574 | >mdo-mir-367 | MI0005375 | >sly-MIR169b | MI0008362 |
| >ame-mir-1 | MI0001575 | >bmo-mir-31 | MI0005377 | >sly-MIR169c | MI0008363 |
| >ame-mir-12 | MI0001576 | >bmo-mir-71 | MI0005378 | >sly-MIR169d | MI0008364 |
| >ame-mir-124 | MI0001577 | >ath-MIR822 | MI0005379 | >sly-MIR171a | MI0008365 |
| >ame-mir-125 | MI0001578 | >ath-MIR823 | MI0005380 | >sly-MIR171b | MI0008366 |
| >ame-mir-133 | MI0001579 | >ath-MIR825 | MI0005381 | >sly-MIR171c | MI0008367 |
| >ame-mir-184 | MI0001580 | >ath-MIR826 | MI0005382 | >sly-MIR171d | MI0008368 |
| >ame-mir-210 | MI0001581 | >ath-MIR827 | MI0005383 | >sly-MIR395a | MI0008369 |
| >ame-mir-219 | MI0001582 | >ath-MIR828 | MI0005384 | >sly-MIR395b | MI0008370 |
| >ame-mir-263 | MI0001583 | >ath-MIR829 | MI0005385 | >sly-MIR397 | MI0008371 |
| >ame-mir-276 | MI0001584 | >ath-MIR830 | MI0005386 | >bmo-mir-1920 | MI0008390 |
| >ame-mir-277 | MI0001585 | >ath-MIR831 | MI0005387 | >bmo-mir-1921 | MI0008391 |
| >ame-mir-278 | MI0001586 | >ath-MIR832 | MI0005388 | >bmo-mir-1922 | MI0008392 |
| >ame-mir-281 | MI0001587 | >ath-MIR833 | MI0005389 | >bmo-mir-1923 | MI0008393 |
| >ame-mir-282 | MI0001588 | >ath-MIR834 | MI0005390 | >bmo-mir-1926 | MI0008394 |
| >ame-mir-2-1 | MI0001589 | >ath-MIR835 | MI0005391 | >bmo-mir-1924 | MI0008395 |
| >ame-mir-2-2 | MI0001590 | >ath-MIR836 | MI0005392 | >bmo-mir-1925 | MI0008396 |
| >ame-mir-305 | MI0001591 | >ath-MIR837 | MI0005393 | >ptr-let-7a-1 | MI0008397 |
| >ame-mir-315 | MI0001592 | >ath-MIR838 | MI0005394 | >ptr-let-7a-2 | MI0008398 |
| >ame-mir-317 | MI0001593 | >ath-MIR839 | MI0005395 | >ptr-let-7a-3 | MI0008399 |
| >ame-mir-7 | MI0001594 | >ath-MIR840 | MI0005396 | >ptr-let-7b | MI0008400 |
| >ame-mir-8 | MI0001595 | >ath-MIR841 | MI0005397 | >ptr-let-7c | MI0008401 |
| >ame-mir-9a | MI0001596 | >ath-MIR842 | MI0005398 | >ptr-let-7d | MI0008402 |
| >ame-mir-9b | MI0001597 | >ath-MIR843 | MI0005399 | >ptr-let-7e | MI0008403 |
| >ame-mir-iab-4 | MI0001598 | >ath-MIR844 | MI0005400 | >ptr-let-7f-1 | MI0008404 |
| >aga-bantam | MI0001599 | >ath-MIR845a | MI0005401 | >ptr-let-7f-2 | MI0008405 |
| >aga-let-7 | MI0001600 | >ath-MIR846 | MI0005402 | >ptr-let-7g | MI0008406 |
| >aga-mir-1 | MI0001601 | >ath-MIR848 | MI0005403 | >ptr-let-7i | MI0008407 |
| >aga-mir-10 | MI0001602 | >ath-MIR849 | MI0005404 | >ptr-mir-101-2 | MI0008408 |
| >aga-mir-100 | MI0001603 | >ath-MIR850 | MI0005405 | >ptr-mir-103-2 | MI0008409 |
| >aga-mir-124 | MI0001604 | >ath-MIR851 | MI0005406 | >ptr-mir-10a | MI0008410 |
| >aga-mir-125 | MI0001605 | >ath-MIR852 | MI0005407 | >ptr-mir-10b | MI0008411 |
| >aga-mir-133 | MI0001606 | >ath-MIR853 | MI0005408 | >ptr-mir-1-1 | MI0008412 |
| >aga-mir-13b | MI0001607 | >ath-MIR824 | MI0005409 | >ptr-mir-1178 | MI0008413 |
| >aga-mir-14 | MI0001608 | >ath-MIR847 | MI0005410 | >ptr-mir-1179 | MI0008414 |
| >aga-mir-184 | MI0001609 | >ath-MIR855 | MI0005411 | >ptr-mir-1181 | MI0008415 |
| >aga-mir-210 | MI0001610 | >ath-MIR854a | MI0005412 | >ptr-mir-1182 | MI0008416 |
| >aga-mir-219 | MI0001611 | >ath-MIR854b | MI0005413 | >ptr-mir-1183 | MI0008417 |
| >aga-mir-263 | MI0001612 | >ath-MIR854c | MI0005414 | >ptr-mir-1184 | MI0008418 |
| >aga-mir-275 | MI0001613 | >ath-MIR854d | MI0005415 | >ptr-mir-1185-1 | MI0008419 |
| >aga-mir-276 | MI0001614 | >hsa-mir-675 | MI0005416 | >ptr-mir-1185-2 | MI0008420 |
| >aga-mir-277 | MI0001615 | >cbr-mir-784 | MI0005417 | >ptr-mir-1197 | MI0008421 |
| >aga-mir-278 | MI0001616 | >cbr-mir-785 | MI0005418 | >ptr-mir-1-2 | MI0008422 |
| >aga-mir-279 | MI0001617 | >cbr-mir-786 | MI0005419 | >ptr-mir-1201 | MI0008423 |
| >aga-mir-281 | MI0001618 | >cbr-mir-787 | MI0005420 | >ptr-mir-1202 | MI0008424 |
| >aga-mir-282 | MI0001619 | >cbr-mir-788 | MI0005421 | >ptr-mir-1203 | MI0008425 |
| >aga-mir-283 | MI0001620 | >cbr-mir-789a | MI0005422 | >ptr-mir-1204 | MI0008426 |
| >aga-mir-2-1 | MI0001621 | >cbr-mir-789b | MI0005423 | >ptr-mir-1205 | MI0008427 |
| >aga-mir-2-2 | MI0001622 | >cbr-mir-790-1 | MI0005424 | >ptr-mir-1206 | MI0008428 |
| >aga-mir-305 | MI0001623 | >cbr-mir-790-2 | MI0005425 | >ptr-mir-1207 | MI0008429 |
| >aga-mir-307 | MI0001624 | >cbr-mir-791 | MI0005426 | >ptr-mir-1208 | MI0008430 |
| >aga-mir-308 | MI0001625 | >cbr-mir-792 | MI0005427 | >ptr-mir-122 | MI0008431 |
| >aga-mir-315 | MI0001626 | >cbr-mir-235 | MI0005428 | >ptr-mir-1225 | MI0008432 |
| >aga-mir-317 | MI0001627 | >cbr-mir-242 | MI0005429 | >ptr-mir-1227 | MI0008433 |
| >aga-mir-7 | MI0001628 | >cbr-mir-255 | MI0005430 | >ptr-mir-1233-1 | MI0008434 |
| >aga-mir-79 | MI0001629 | >cbr-mir-359 | MI0005431 | >ptr-mir-1233-2 | MI0008435 |
| >aga-mir-8 | MI0001630 | >cbr-mir-392 | MI0005432 | >ptr-mir-1233-3 | MI0008436 |
| >aga-mir-9a | MI0001631 | >ath-MIR856 | MI0005433 | >ptr-mir-1234 | MI0008437 |
| >aga-mir-92a | MI0001632 | >ath-MIR857 | MI0005434 | >ptr-mir-1236 | MI0008438 |
| >aga-mir-92b | MI0001633 | >ath-MIR858 | MI0005435 | >ptr-mir-1237 | MI0008439 |
| >aga-mir-9b | MI0001634 | >ath-MIR859 | MI0005436 | >ptr-mir-1244-1 | MI0008440 |
| >aga-mir-9c | MI0001635 | >ath-MIR860 | MI0005437 | >ptr-mir-1244-2 | MI0008441 |
| >aga-mir-iab-4 | MI0001636 | >ath-MIR861 | MI0005438 | >ptr-mir-1244-3 | MI0008442 |
| >hsa-mir-448 | MI0001637 | >ath-MIR862 | MI0005439 | >ptr-mir-1244-4 | MI0008443 |
| >mmu-mir-448 | MI0001638 | >ath-MIR863 | MI0005440 | >ptr-mir-1244-5 | MI0008444 |
| >rno-mir-448 | MI0001639 | >ath-MIR864 | MI0005441 | >ptr-mir-1244-6 | MI0008445 |
| >cfa-mir-448 | MI0001640 | >ath-MIR865 | MI0005442 | >ptr-mir-1244-7 | MI0008446 |
| >hsa-mir-429 | MI0001641 | >ath-MIR866 | MI0005443 | >ptr-mir-1244-8 | MI0008447 |
| >mmu-mir-429 | MI0001642 | >ath-MIR845b | MI0005444 | >ptr-mir-1244-9 | MI0008448 |
| >rno-mir-429 | MI0001643 | >ath-MIR867 | MI0005445 | >ptr-mir-1244-10 | MI0008449 |
| >cfa-mir-429 | MI0001644 | >ath-MIR868 | MI0005446 | >ptr-mir-1244-11 | MI0008450 |
| >mmu-mir-365-2 | MI0001645 | >ath-MIR869 | MI0005447 | >ptr-mir-1245 | MI0008451 |
| >cfa-mir-365-2 | MI0001647 | >ath-MIR870 | MI0005448 | >ptr-mir-1246 | MI0008452 |
| >hsa-mir-449a | MI0001648 | >mmu-mir-181d | MI0005450 | >ptr-mir-1247 | MI0008453 |
| >mmu-mir-449a | MI0001649 | >bta-let-7a-2 | MI0005451 | >ptr-mir-1248 | MI0008454 |
| >rno-mir-449a | MI0001650 | >bta-let-7a-3 | MI0005452 | >ptr-mir-1249 | MI0008455 |
| >cfa-mir-449 | MI0001651 | >bta-let-7b | MI0005453 | >ptr-mir-1250 | MI0008456 |
| >hsa-mir-450a-1 | MI0001652 | >bta-let-7c | MI0005454 | >ptr-mir-1251 | MI0008457 |
| >mmu-mir-450a-1 | MI0001653 | >bta-let-7e | MI0005455 | >ptr-mir-1253 | MI0008458 |
| >rno-mir-450a | MI0001654 | >bta-mir-103-2 | MI0005456 | >ptr-mir-1254 | MI0008459 |
| >cfa-mir-450a | MI0001655 | >bta-mir-125b-2 | MI0005457 | >ptr-mir-1255b | MI0008460 |
| >rno-mir-365 | MI0001656 | >bta-mir-15a | MI0005458 | >ptr-mir-1256 | MI0008461 |
| >cfa-mir-365-1 | MI0001657 | >bta-mir-195 | MI0005459 | >ptr-mir-1258 | MI0008462 |
| >mghv-mir-M1-1 | MI0001669 | >bta-mir-19a | MI0005460 | >ptr-mir-1259 | MI0008463 |
| >mghv-mir-M1-2 | MI0001670 | >bta-mir-19b | MI0005461 | >ptr-mir-125a | MI0008464 |
| >mghv-mir-M1-3 | MI0001671 | >bta-mir-204 | MI0005462 | >ptr-mir-126 | MI0008465 |
| >mghv-mir-M1-4 | MI0001672 | >bta-mir-331 | MI0005463 | >ptr-mir-1262 | MI0008466 |
| >mghv-mir-M1-5 | MI0001673 | >bta-mir-34a | MI0005464 | >ptr-mir-1263 | MI0008467 |
| >mghv-mir-M1-6 | MI0001674 | >bta-mir-365 | MI0005465 | >ptr-mir-1264 | MI0008468 |
| >mghv-mir-M1-7 | MI0001675 | >bta-mir-374a | MI0005466 | >ptr-mir-1265 | MI0008469 |
| >mghv-mir-M1-8 | MI0001676 | >bta-mir-497 | MI0005467 | >ptr-mir-1266 | MI0008470 |
| >mghv-mir-M1-9 | MI0001677 | >bta-mir-660 | MI0005468 | >ptr-mir-1267 | MI0008471 |
| >hcmv-mir-UL22A | MI0001678 | >bta-mir-99b | MI0005469 | >ptr-mir-1271 | MI0008472 |
| >hcmv-mir-UL36 | MI0001679 | >mmu-mir-743b | MI0005470 | >ptr-mir-1272 | MI0008473 |
| >hcmv-mir-UL112 | MI0001680 | >mmu-mir-871 | MI0005471 | >ptr-mir-1273 | MI0008474 |
| >hcmv-mir-UL148D | MI0001681 | >mmu-mir-879 | MI0005472 | >ptr-mir-1274b | MI0008475 |
| >hcmv-mir-US5-1 | MI0001682 | >mmu-mir-880 | MI0005473 | >ptr-mir-1275 | MI0008476 |
| >hcmv-mir-US5-2 | MI0001683 | >mmu-mir-881 | MI0005474 | >ptr-mir-1276 | MI0008477 |
| >hcmv-mir-US25-1 | MI0001684 | >mmu-mir-882 | MI0005475 | >ptr-mir-1278 | MI0008478 |
| >hcmv-mir-US25-2 | MI0001685 | >mmu-mir-883a | MI0005476 | >ptr-mir-1280 | MI0008479 |
| >hcmv-mir-US33 | MI0001686 | >mmu-mir-883b | MI0005477 | >ptr-mir-1281 | MI0008480 |
| >osa-MIR435 | MI0001687 | >mmu-mir-190b | MI0005478 | >ptr-mir-1282-1 | MI0008481 |
| >osa-MIR437 | MI0001688 | >mmu-mir-874 | MI0005479 | >ptr-mir-128-2 | MI0008482 |
| >osa-MIR438 | MI0001689 | >mmu-mir-876 | MI0005480 | >ptr-mir-1282-2 | MI0008483 |
| >osa-MIR390 | MI0001690 | >mmu-mir-105 | MI0005481 | >ptr-mir-1283 | MI0008484 |
| >osa-MIR439a | MI0001691 | >mmu-mir-147 | MI0005482 | >ptr-mir-1284 | MI0008485 |
| >osa-MIR439b | MI0001692 | >mmu-mir-18b | MI0005483 | >ptr-mir-1285 | MI0008486 |
| >osa-MIR439c | MI0001693 | >mmu-mir-193b | MI0005484 | >ptr-mir-1286 | MI0008487 |
| >osa-MIR439d | MI0001694 | >mmu-mir-197 | MI0005485 | >ptr-mir-1288 | MI0008488 |
| >osa-MIR439e | MI0001695 | >mmu-mir-220 | MI0005487 | >ptr-mir-1289-1 | MI0008489 |
| >osa-MIR439f | MI0001696 | >mmu-mir-297a-3 | MI0005488 | >ptr-mir-1289-2 | MI0008490 |
| >osa-MIR439g | MI0001697 | >mmu-mir-297a-4 | MI0005489 | >ptr-mir-1290 | MI0008491 |
| >osa-MIR439h | MI0001698 | >mmu-mir-297a-5 | MI0005490 | >ptr-mir-1291 | MI0008492 |
| >osa-MIR439i | MI0001699 | >mmu-mir-297a-6 | MI0005491 | >ptr-mir-129-1 | MI0008493 |
| >osa-MIR439j | MI0001700 | >mmu-mir-297c | MI0005492 | >ptr-mir-129-2 | MI0008494 |
| >osa-MIR440 | MI0001701 | >mmu-mir-327 | MI0005493 | >ptr-mir-1292 | MI0008495 |
| >osa-MIR396d | MI0001702 | >mmu-mir-343 | MI0005494 | >ptr-mir-1293 | MI0008496 |
| >osa-MIR396e | MI0001703 | >mmu-mir-344-2 | MI0005495 | >ptr-mir-1294 | MI0008497 |
| >osa-MIR441a | MI0001704 | >mmu-mir-421 | MI0005496 | >ptr-mir-1295 | MI0008498 |
| >osa-MIR441b | MI0001705 | >mmu-mir-453 | MI0005497 | >ptr-mir-1296 | MI0008499 |
| >osa-MIR441c | MI0001706 | >mmu-mir-465b-1 | MI0005498 | >ptr-mir-1297-1 | MI0008500 |
| >osa-MIR442 | MI0001707 | >mmu-mir-465b-2 | MI0005499 | >ptr-mir-1297-2 | MI0008501 |
| >osa-MIR443 | MI0001708 | >mmu-mir-465c-1 | MI0005500 | >ptr-mir-1298 | MI0008502 |
| >osa-MIR445a | MI0001709 | >mmu-mir-465c-2 | MI0005501 | >ptr-mir-1299 | MI0008503 |
| >osa-MIR445b | MI0001710 | >mmu-mir-466b-1 | MI0005502 | >ptr-mir-1300b | MI0008504 |
| >osa-MIR445c | MI0001711 | >mmu-mir-466b-2 | MI0005503 | >ptr-mir-1300a-3 | MI0008505 |
| >osa-MIR445d | MI0001712 | >mmu-mir-466b-3 | MI0005504 | >ptr-mir-1300a-4 | MI0008506 |
| >osa-MIR445e | MI0001713 | >mmu-mir-466c | MI0005505 | >ptr-mir-1300a-1 | MI0008507 |
| >osa-MIR445f | MI0001714 | >mmu-mir-466e | MI0005506 | >ptr-mir-1300a-5 | MI0008508 |
| >osa-MIR445g | MI0001715 | >mmu-mir-466f-1 | MI0005507 | >ptr-mir-1300a-6 | MI0008509 |
| >osa-MIR445h | MI0001716 | >mmu-mir-466f-2 | MI0005508 | >ptr-mir-1300a-7 | MI0008510 |
| >osa-MIR445i | MI0001717 | >mmu-mir-466f-3 | MI0005509 | >ptr-mir-1300a-8 | MI0008511 |
| >osa-MIR446 | MI0001718 | >mmu-mir-466g | MI0005510 | >ptr-mir-1300a-9 | MI0008512 |
| >osa-MIR444a | MI0001719 | >mmu-mir-466h | MI0005511 | >ptr-mir-1300a-10 | MI0008513 |
| >dre-mir-429 | MI0001720 | >mmu-mir-467c | MI0005512 | >ptr-mir-1300a-11 | MI0008514 |
| >hsa-mir-431 | MI0001721 | >mmu-mir-467d | MI0005513 | >ptr-mir-1300a-2 | MI0008515 |
| >rno-mir-431 | MI0001722 | >mmu-mir-493 | MI0005514 | >ptr-mir-1302-1 | MI0008516 |
| >hsa-mir-433 | MI0001723 | >mmu-mir-504 | MI0005515 | >ptr-mir-1302-2 | MI0008517 |
| >rno-mir-433 | MI0001724 | >mmu-mir-509 | MI0005516 | >ptr-mir-1302-3 | MI0008518 |
| >hsa-mir-329-1 | MI0001725 | >mmu-mir-568 | MI0005517 | >ptr-mir-1302-4 | MI0008519 |
| >hsa-mir-329-2 | MI0001726 | >mmu-mir-574 | MI0005518 | >ptr-mir-1302-5 | MI0008520 |
| >hsa-mir-453 | MI0001727 | >mmu-mir-590 | MI0005519 | >ptr-mir-1302-6 | MI0008521 |
| >hsa-mir-451 | MI0001729 | >mmu-mir-654 | MI0005520 | >ptr-mir-1302-7 | MI0008522 |
| >mmu-mir-451 | MI0001730 | >mmu-mir-92b | MI0005521 | >ptr-mir-1302-8 | MI0008523 |
| >rno-mir-451 | MI0001731 | >hsa-mir-298 | MI0005523 | >ptr-mir-1303 | MI0008524 |
| >dre-mir-451 | MI0001732 | >hsa-mir-891a | MI0005524 | >ptr-mir-1306 | MI0008525 |
| >hsa-mir-452 | MI0001733 | >hsa-mir-300 | MI0005525 | >ptr-mir-1307 | MI0008526 |
| >mmu-mir-452 | MI0001734 | >hsa-mir-886 | MI0005527 | >ptr-mir-130a | MI0008527 |
| >hsa-mir-409 | MI0001735 | >hsa-mir-892a | MI0005528 | >ptr-mir-130b | MI0008528 |
| >mtr-MIR162 | MI0001738 | >hsa-mir-220b | MI0005529 | >ptr-mir-1322 | MI0008529 |
| >mtr-MIR160 | MI0001739 | >hsa-mir-509-2 | MI0005530 | >ptr-mir-1323 | MI0008530 |
| >mtr-MIR166 | MI0001740 | >hsa-mir-450b | MI0005531 | >ptr-mir-1324-1 | MI0008531 |
| >mtr-MIR169a | MI0001741 | >hsa-mir-874 | MI0005532 | >ptr-mir-1324-2 | MI0008532 |
| >mtr-MIR169b | MI0001742 | >hsa-mir-890 | MI0005533 | >ptr-mir-133a-2 | MI0008533 |
| >mtr-MIR399b | MI0001743 | >hsa-mir-891b | MI0005534 | >ptr-mir-133b | MI0008534 |
| >mtr-MIR399d | MI0001744 | >hsa-mir-220c | MI0005536 | >ptr-mir-134 | MI0008535 |
| >mtr-MIR393 | MI0001745 | >hsa-mir-888 | MI0005537 | >ptr-mir-135a-1 | MI0008536 |
| >mtr-MIR395a | MI0001746 | >hsa-mir-892b | MI0005538 | >ptr-mir-135b | MI0008537 |
| >mtr-MIR395b | MI0001747 | >hsa-mir-541 | MI0005539 | >ptr-mir-137 | MI0008538 |
| >mtr-MIR399c | MI0001748 | >hsa-mir-889 | MI0005540 | >ptr-mir-138 | MI0008539 |
| >mtr-MIR399a | MI0001749 | >hsa-mir-875 | MI0005541 | >ptr-mir-139 | MI0008540 |
| >mtr-MIR399e | MI0001750 | >hsa-mir-876 | MI0005542 | >ptr-mir-141-1 | MI0008541 |
| >mtr-MIR319 | MI0001751 | >hsa-mir-708 | MI0005543 | >ptr-mir-141-2 | MI0008542 |
| >mtr-MIR156 | MI0001752 | >hsa-mir-147b | MI0005544 | >ptr-mir-142 | MI0008543 |
| >mtr-MIR171 | MI0001753 | >hsa-mir-190b | MI0005545 | >ptr-mir-146a | MI0008544 |
| >sof-MIR156 | MI0001754 | >mmu-mir-466d | MI0005546 | >ptr-mir-146b | MI0008545 |
| >sof-MIR396 | MI0001755 | >mmu-mir-449b | MI0005547 | >ptr-mir-147b | MI0008546 |
| >sof-MIR159a | MI0001756 | >mmu-mir-878 | MI0005548 | >ptr-mir-148a | MI0008547 |
| >sof-MIR159b | MI0001757 | >mmu-mir-872 | MI0005549 | >ptr-mir-148b | MI0008548 |
| >sof-MIR159d | MI0001758 | >mmu-mir-873 | MI0005550 | >ptr-mir-149 | MI0008549 |
| >sof-MIR159e | MI0001759 | >mmu-mir-875 | MI0005551 | >ptr-mir-150 | MI0008550 |
| >sof-MIR159c | MI0001760 | >mmu-mir-208b | MI0005552 | >ptr-mir-151 | MI0008551 |
| >sof-MIR167a | MI0001761 | >mmu-mir-877 | MI0005553 | >ptr-mir-152 | MI0008552 |
| >sof-MIR167b | MI0001762 | >mmu-mir-511 | MI0005554 | >ptr-mir-153 | MI0008553 |
| >sof-MIR168a | MI0001763 | >mmu-mir-544 | MI0005555 | >ptr-mir-155 | MI0008554 |
| >sof-MIR168b | MI0001764 | >mmu-mir-598 | MI0005556 | >ptr-mir-16-2 | MI0008555 |
| >sof-MIR408a | MI0001765 | >mmu-mir-653 | MI0005557 | >ptr-mir-181b-2 | MI0008556 |
| >sof-MIR408b | MI0001766 | >hsa-mir-744 | MI0005559 | >ptr-mir-181d | MI0008557 |
| >sof-MIR408c | MI0001767 | >hsa-mir-885 | MI0005560 | >ptr-mir-182 | MI0008558 |
| >sof-MIR408d | MI0001768 | >hsa-mir-877 | MI0005561 | >ptr-mir-1825 | MI0008559 |
| >sof-MIR408e | MI0001769 | >hsa-mir-887 | MI0005562 | >ptr-mir-1827 | MI0008560 |
| >gma-MIR156d | MI0001770 | >hsa-mir-665 | MI0005563 | >ptr-mir-185 | MI0008561 |
| >gma-MIR156e | MI0001771 | >hsa-mir-873 | MI0005564 | >ptr-mir-18b | MI0008562 |
| >gma-MIR156c | MI0001772 | >hsa-mir-543 | MI0005565 | >ptr-mir-190b | MI0008563 |
| >gma-MIR159a | MI0001773 | >hsa-mir-374b | MI0005566 | >ptr-mir-191 | MI0008564 |
| >gma-MIR160 | MI0001774 | >hsa-mir-760 | MI0005567 | >ptr-mir-192 | MI0008565 |
| >gma-MIR166a | MI0001775 | >hsa-mir-301b | MI0005568 | >ptr-mir-193a | MI0008566 |
| >gma-MIR166b | MI0001776 | >hsa-mir-216b | MI0005569 | >ptr-mir-193b | MI0008567 |
| >gma-MIR167a | MI0001777 | >hsa-mir-208b | MI0005570 | >ptr-mir-195 | MI0008568 |
| >gma-MIR167b | MI0001778 | >ghr-MIR156a | MI0005638 | >ptr-mir-196a-1 | MI0008569 |
| >gma-MIR168 | MI0001779 | >ghr-MIR156b | MI0005639 | >ptr-mir-196b | MI0008570 |
| >gma-MIR172a | MI0001780 | >ghr-MIR156c | MI0005640 | >ptr-mir-199a-1 | MI0008571 |
| >gma-MIR172b | MI0001781 | >ghr-MIR156d | MI0005641 | >ptr-mir-199b | MI0008572 |
| >gma-MIR319a | MI0001782 | >gra-MIR157a | MI0005642 | >ptr-mir-200a | MI0008573 |
| >gma-MIR319b | MI0001783 | >gra-MIR157b | MI0005643 | >ptr-mir-202 | MI0008574 |
| >gma-MIR156a | MI0001784 | >ghr-MIR162a | MI0005644 | >ptr-mir-203 | MI0008575 |
| >gma-MIR396a | MI0001785 | >ghr-MIR166b | MI0005645 | >ptr-mir-206 | MI0008576 |
| >gma-MIR396b | MI0001786 | >ghb-MIR169a | MI0005646 | >ptr-mir-208a | MI0008577 |
| >gma-MIR398a | MI0001787 | >ghr-MIR390a | MI0005647 | >ptr-mir-208b | MI0008578 |
| >gma-MIR398b | MI0001788 | >ghr-MIR390b | MI0005648 | >ptr-mir-20b | MI0008579 |
| >gma-MIR319c | MI0001789 | >ghr-MIR390c | MI0005649 | >ptr-mir-210 | MI0008580 |
| >gma-MIR156b | MI0001790 | >ghr-MIR396a | MI0005650 | >ptr-mir-211 | MI0008581 |
| >gma-MIR169a | MI0001791 | >ghr-MIR396b | MI0005651 | >ptr-mir-216b | MI0008582 |
| >zma-MIR171d | MI0001792 | >ghr-MIR399a | MI0005652 | >ptr-mir-217 | MI0008583 |
| >zma-MIR171f | MI0001793 | >ghr-MIR399b | MI0005653 | >ptr-mir-219-1 | MI0008584 |
| >zma-MIR394a | MI0001794 | >ppt-MIR156c | MI0005654 | >ptr-mir-219-2 | MI0008585 |
| >zma-MIR394b | MI0001795 | >ppt-MIR160a | MI0005655 | >ptr-mir-220b | MI0008586 |
| >zma-MIR395b | MI0001796 | >ppt-MIR160b | MI0005656 | >ptr-mir-221 | MI0008587 |
| >zma-MIR395c | MI0001798 | >ppt-MIR160c | MI0005657 | >ptr-mir-24-1 | MI0008588 |
| >zma-MIR395a | MI0001799 | >ppt-MIR160d | MI0005658 | >ptr-mir-26a-2 | MI0008589 |
| >zma-MIR396b | MI0001800 | >ppt-MIR166b | MI0005659 | >ptr-mir-26b | MI0008590 |
| >zma-MIR396a | MI0001801 | >ppt-MIR166a | MI0005660 | >ptr-mir-27b | MI0008591 |
| >zma-MIR399a | MI0001802 | >ppt-MIR167 | MI0005661 | >ptr-mir-296 | MI0008592 |
| >zma-MIR399c | MI0001803 | >ppt-MIR319e | MI0005665 | >ptr-mir-297 | MI0008593 |
| >zma-MIR399b | MI0001804 | >ppt-MIR395 | MI0005667 | >ptr-mir-298 | MI0008594 |
| >zma-MIR399d | MI0001805 | >ppt-MIR408 | MI0005668 | >ptr-mir-29c | MI0008595 |
| >zma-MIR399e | MI0001806 | >ppt-MIR414 | MI0005669 | >ptr-mir-300 | MI0008596 |
| >zma-MIR399f | MI0001807 | >ppt-MIR419 | MI0005670 | >ptr-mir-301a | MI0008597 |
| >zma-MIR156j | MI0001808 | >ppt-MIR477c | MI0005671 | >ptr-mir-301b | MI0008598 |
| >zma-MIR159a | MI0001809 | >ppt-MIR477a | MI0005672 | >ptr-mir-302a | MI0008599 |
| >zma-MIR159b | MI0001810 | >ppt-MIR477f | MI0005673 | >ptr-mir-302b | MI0008600 |
| >zma-MIR159c | MI0001811 | >ppt-MIR477g | MI0005674 | >ptr-mir-302c | MI0008601 |
| >zma-MIR159d | MI0001812 | >ppt-MIR534b | MI0005675 | >ptr-mir-302d | MI0008602 |
| >zma-MIR319a | MI0001813 | >ppt-MIR893 | MI0005676 | >ptr-mir-302e | MI0008603 |
| >zma-MIR319c | MI0001814 | >ppt-MIR894 | MI0005677 | >ptr-mir-302f | MI0008604 |
| >zma-MIR319b | MI0001815 | >ppt-MIR895 | MI0005678 | >ptr-mir-30c-1 | MI0008605 |
| >zma-MIR319d | MI0001816 | >ppt-MIR896 | MI0005680 | >ptr-mir-30e | MI0008606 |
| >zma-MIR166k | MI0001817 | >ppt-MIR897 | MI0005681 | >ptr-mir-320a | MI0008607 |
| >zma-MIR166j | MI0001818 | >ppt-MIR899 | MI0005682 | >ptr-mir-320b-1 | MI0008608 |
| >zma-MIR167e | MI0001819 | >ppt-MIR900 | MI0005683 | >ptr-mir-320b-2 | MI0008609 |
| >zma-MIR167f | MI0001820 | >ppt-MIR901 | MI0005684 | >ptr-mir-320c-1 | MI0008610 |
| >zma-MIR167g | MI0001821 | >ppt-MIR902a | MI0005685 | >ptr-mir-320c-2 | MI0008611 |
| >zma-MIR167h | MI0001822 | >ppt-MIR902b | MI0005686 | >ptr-mir-320d-1 | MI0008612 |
| >zma-MIR167i | MI0001823 | >ppt-MIR536c | MI0005687 | >ptr-mir-320d-2 | MI0008613 |
| >zma-MIR168a | MI0001824 | >ppt-MIR171a | MI0005688 | >ptr-mir-320d-3 | MI0008614 |
| >zma-MIR168b | MI0001825 | >ppt-MIR171b | MI0005689 | >ptr-mir-323 | MI0008615 |
| >zma-MIR169c | MI0001826 | >ppt-MIR903 | MI0005690 | >ptr-mir-324 | MI0008616 |
| >zma-MIR169f | MI0001827 | >ppt-MIR536b | MI0005691 | >ptr-mir-326 | MI0008617 |
| >zma-MIR169g | MI0001828 | >ppt-MIR904a | MI0005692 | >ptr-mir-328 | MI0008618 |
| >zma-MIR169h | MI0001829 | >ppt-MIR904b | MI0005693 | >ptr-mir-329-1 | MI0008619 |
| >zma-MIR169i | MI0001830 | >ppt-MIR898b | MI0005694 | >ptr-mir-329-2 | MI0008620 |
| >zma-MIR169k | MI0001831 | >ppt-MIR898a | MI0005695 | >ptr-mir-330 | MI0008621 |
| >zma-MIR169j | MI0001832 | >ppt-MIR156b | MI0005696 | >ptr-mir-331 | MI0008622 |
| >zma-MIR169d | MI0001833 | >cre-MIR918 | MI0005697 | >ptr-mir-335 | MI0008623 |
| >zma-MIR169e | MI0001834 | >cre-MIR905 | MI0005698 | >ptr-mir-337 | MI0008624 |
| >zma-MIR171c | MI0001835 | >cre-MIR906 | MI0005699 | >ptr-mir-338 | MI0008625 |
| >zma-MIR171j | MI0001836 | >cre-MIR907 | MI0005700 | >ptr-mir-339 | MI0008626 |
| >zma-MIR171e | MI0001837 | >cre-MIR908 | MI0005701 | >ptr-mir-33b | MI0008627 |
| >zma-MIR171i | MI0001838 | >cre-MIR909 | MI0005702 | >ptr-mir-340 | MI0008628 |
| >zma-MIR171g | MI0001839 | >cre-MIR910 | MI0005703 | >ptr-mir-342 | MI0008629 |
| >zma-MIR172e | MI0001840 | >cre-MIR911 | MI0005704 | >ptr-mir-345 | MI0008630 |
| >zma-MIR1661 | MI0001841 | >cre-MIR912 | MI0005705 | >ptr-mir-346 | MI0008631 |
| >zma-MIR166m | MI0001842 | >cre-MIR913 | MI0005706 | >ptr-mir-34b | MI0008632 |
| >zma-MIR171k | MI0001843 | >cre-MIR914 | MI0005707 | >ptr-mir-34c | MI0008633 |
| >zma-MIR171h | MI0001844 | >cre-MIR915 | MI0005708 | >ptr-mir-361 | MI0008634 |
| >zma-MIR393 | MI0001845 | >cre-MIR919 | MI0005709 | >ptr-mir-362 | MI0008635 |
| >zma-MIR408 | MI0001846 | >cre-MIR917 | MI0005710 | >ptr-mir-365-1 | MI0008636 |
| >zma-MIR156k | MI0001847 | >cre-MIR916 | MI0005711 | >ptr-mir-365-2 | MI0008637 |
| >zma-MIR160f | MI0001848 | >hsa-mir-920 | MI0005712 | >ptr-mir-367 | MI0008638 |
| >sbi-MIR399h | MI0001849 | >hsa-mir-921 | MI0005713 | >ptr-mir-369 | MI0008639 |
| >sbi-MIR399i | MI0001850 | >hsa-mir-922 | MI0005714 | >ptr-mir-370 | MI0008640 |
| >sbi-MIR159b | MI0001851 | >hsa-mir-923 | MI0005715 | >ptr-mir-371 | MI0008641 |
| >sbi-MIR164c | MI0001852 | >hsa-mir-924 | MI0005716 | >ptr-mir-372 | MI0008642 |
| >sbi-MIR166g | MI0001853 | >hsa-mir-509-3 | MI0005717 | >ptr-mir-374a | MI0008643 |
| >sbi-MIR171f | MI0001854 | >rrv-miR-rR1-1 | MI0005718 | >ptr-mir-374b | MI0008644 |
| >sbi-MIR395f | MI0001855 | >rrv-miR-rR1-2 | MI0005719 | >ptr-mir-375 | MI0008645 |
| >sbi-MIR156e | MI0001856 | >rrv-miR-rR1-3 | MI0005720 | >ptr-mir-376a-1 | MI0008646 |
| >dre-let-7a-1 | MI0001857 | >rrv-miR-rR1-4 | MI0005721 | >ptr-mir-376a-2 | MI0008647 |
| >dre-let-7a-2 | MI0001858 | >rrv-miR-rR1-5 | MI0005722 | >ptr-mir-376b | MI0008648 |
| >dre-let-7a-3 | MI0001860 | >rrv-miR-rR1-6 | MI0005723 | >ptr-mir-376c | MI0008649 |
| >dre-let-7a-4 | MI0001861 | >rrv-miR-rR1-7 | MI0005724 | >ptr-mir-377 | MI0008650 |
| >dre-let-7a-5 | MI0001862 | >cgr-mir-21 | MI0005725 | >ptr-mir-378 | MI0008651 |
| >dre-let-7a-6 | MI0001863 | >ame-let-7 | MI0005726 | >ptr-mir-379 | MI0008652 |
| >dre-let-7b | MI0001865 | >ame-mir-10 | MI0005727 | >ptr-mir-380 | MI0008653 |
| >dre-let-7c-1 | MI0001866 | >ame-mir-100 | MI0005728 | >ptr-mir-381 | MI0008654 |
| >dre-let-7c-2 | MI0001867 | >ame-mir-137 | MI0005729 | >ptr-mir-382 | MI0008655 |
| >dre-let-7d-1 | MI0001868 | >ame-mir-13a | MI0005730 | >ptr-mir-383 | MI0008656 |
| >dre-let-7d-2 | MI0001870 | >ame-mir-14 | MI0005731 | >ptr-mir-409 | MI0008657 |
| >dre-let-7e | MI0001871 | >ame-mir-2-3 | MI0005732 | >ptr-mir-410 | MI0008658 |
| >dre-let-7f | MI0001872 | >ame-mir-275 | MI0005733 | >ptr-mir-411 | MI0008659 |
| >dre-let-7g-1 | MI0001873 | >ame-mir-279 | MI0005734 | >ptr-mir-412 | MI0008660 |
| >dre-let-7g-2 | MI0001874 | >ame-mir-283 | MI0005735 | >ptr-mir-421 | MI0008661 |
| >dre-let-7h | MI0001875 | >ame-mir-29b | MI0005736 | >ptr-mir-422a | MI0008662 |
| >dre-let-7i | MI0001876 | >ame-mir-31a | MI0005737 | >ptr-mir-423 | MI0008663 |
| >dre-mir-1-2 | MI0001877 | >ame-mir-33 | MI0005738 | >ptr-mir-424 | MI0008664 |
| >dre-mir-1-1 | MI0001878 | >ame-mir-34 | MI0005739 | >ptr-mir-425 | MI0008665 |
| >dre-mir-7a-3 | MI0001879 | >ame-mir-375 | MI0005740 | >ptr-mir-431 | MI0008666 |
| >dre-mir-9-1 | MI0001880 | >ame-mir-71 | MI0005741 | >ptr-mir-432 | MI0008667 |
| >dre-mir-9-2 | MI0001881 | >ame-mir-79 | MI0005742 | >ptr-mir-433 | MI0008668 |
| >dre-mir-9-4 | MI0001882 | >ame-mir-87-1 | MI0005743 | >ptr-mir-448 | MI0008669 |
| >dre-mir-9-3 | MI0001883 | >ame-mir-87-2 | MI0005744 | >ptr-mir-449a | MI0008670 |
| >dre-mir-9-5 | MI0001884 | >ame-mir-92a | MI0005745 | >ptr-mir-449b | MI0008671 |
| >dre-mir-9-6 | MI0001885 | >ame-mir-925 | MI0005746 | >ptr-mir-450a-1 | MI0008672 |
| >dre-mir-9-7 | MI0001886 | >ame-mir-926 | MI0005747 | >ptr-mir-450a-2 | MI0008673 |
| >dre-mir-10b-2 | MI0001887 | >ame-mir-927 | MI0005748 | >ptr-mir-450b | MI0008674 |
| >dre-mir-10c | MI0001888 | >ame-mir-928 | MI0005749 | >ptr-mir-451 | MI0008675 |
| >dre-mir-10d-1 | MI0001889 | >ame-mir-190 | MI0005750 | >ptr-mir-452 | MI0008676 |
| >dre-mir-10d-2 | MI0001890 | >ame-mir-929 | MI0005751 | >ptr-mir-453 | MI0008677 |
| >dre-mir-15a-1 | MI0001891 | >ame-mir-930 | MI0005752 | >ptr-mir-454 | MI0008678 |
| >dre-mir-15a-2 | MI0001892 | >ame-mir-931 | MI0005753 | >ptr-mir-455 | MI0008679 |
| >dre-mir-15b | MI0001893 | >ame-mir-932 | MI0005754 | >ptr-mir-484 | MI0008680 |
| >dre-mir-16a | MI0001894 | >hsa-mir-933 | MI0005755 | >ptr-mir-485 | MI0008681 |
| >dre-mir-16b | MI0001895 | >hsa-mir-934 | MI0005756 | >ptr-mir-486 | MI0008682 |
| >dre-mir-16c | MI0001896 | >hsa-mir-935 | MI0005757 | >ptr-mir-487a | MI0008683 |
| >dre-mir-17a-1 | MI0001897 | >hsa-mir-936 | MI0005758 | >ptr-mir-487b | MI0008684 |
| >dre-mir-17a-2 | MI0001898 | >hsa-mir-937 | MI0005759 | >ptr-mir-488 | MI0008685 |
| >dre-mir-20b | MI0001899 | >hsa-mir-938 | MI0005760 | >ptr-mir-489 | MI0008686 |
| >dre-mir-18a | MI0001900 | >hsa-mir-939 | MI0005761 | >ptr-mir-490 | MI0008687 |
| >dre-mir-18b | MI0001901 | >hsa-mir-940 | MI0005762 | >ptr-mir-491 | MI0008688 |
| >dre-mir-18c | MI0001902 | >hsa-mir-941-1 | MI0005763 | >ptr-mir-492 | MI0008689 |
| >dre-mir-19a | MI0001903 | >hsa-mir-941-2 | MI0005764 | >ptr-mir-494 | MI0008690 |
| >dre-mir-19b | MI0001904 | >hsa-mir-941-3 | MI0005765 | >ptr-mir-495 | MI0008691 |
| >dre-mir-19c | MI0001905 | >hsa-mir-941-4 | MI0005766 | >ptr-mir-496 | MI0008692 |
| >dre-mir-19d | MI0001906 | >hsa-mir-942 | MI0005767 | >ptr-mir-498 | MI0008693 |
| >dre-mir-20a | MI0001907 | >hsa-mir-943 | MI0005768 | >ptr-mir-499 | MI0008694 |
| >dre-mir-21-1 | MI0001908 | >hsa-mir-944 | MI0005769 | >ptr-mir-500 | MI0008695 |
| >dre-mir-21-2 | MI0001909 | >bna-MIR156a | MI0005770 | >ptr-mir-501 | MI0008696 |
| >dre-mir-22a-1 | MI0001910 | >bna-MIR171g | MI0005771 | >ptr-mir-502 | MI0008697 |
| >dre-mir-22a-2 | MI0001911 | >bna-MIR393 | MI0005772 | >ptr-mir-503 | MI0008698 |
| >dre-mir-22b | MI0001912 | >bna-MIR396a | MI0005773 | >ptr-mir-504 | MI0008699 |
| >dre-mir-23a-1 | MI0001913 | >bna-MIR399 | MI0005774 | >ptr-mir-505 | MI0008700 |
| >dre-mir-23a-2 | MI0001915 | >hsa-mir-297 | MI0005775 | >ptr-mir-511 | MI0008701 |
| >dre-mir-23a-3 | MI0001916 | >pta-MIR156a | MI0005776 | >ptr-mir-512 | MI0008702 |
| >dre-mir-23b | MI0001917 | >pta-MIR156b | MI0005777 | >ptr-mir-515-1 | MI0008703 |
| >dre-mir-24-4 | MI0001918 | >pta-MIR159a | MI0005778 | >ptr-mir-515-2 | MI0008704 |
| >dre-mir-24-2 | MI0001919 | >pta-MIR159b | MI0005779 | >ptr-mir-516a-1 | MI0008705 |
| >dre-mir-24-3 | MI0001920 | >pta-MIR159c | MI0005780 | >ptr-mir-516a-2 | MI0008706 |
| >dre-mir-24-1 | MI0001921 | >pta-MIR166a | MI0005782 | >ptr-mir-516b-1 | MI0008707 |
| >dre-mir-25 | MI0001922 | >pta-MIR166b | MI0005783 | >ptr-mir-516b-2 | MI0008708 |
| >dre-mir-26a-1 | MI0001923 | >pta-MIR166c | MI0005784 | >ptr-mir-517a | MI0008709 |
| >dre-mir-26a-2 | MI0001925 | >pta-MIR171 | MI0005785 | >ptr-mir-517b-1 | MI0008710 |
| >dre-mir-26a-3 | MI0001926 | >pta-MIR319 | MI0005786 | >ptr-mir-517b-2 | MI0008711 |
| >dre-mir-26b | MI0001927 | >pta-MIR390 | MI0005787 | >ptr-mir-518a | MI0008712 |
| >dre-mir-27a | MI0001928 | >pta-MIR396 | MI0005788 | >ptr-mir-518b | MI0008713 |
| >dre-mir-27b | MI0001929 | >pta-MIR398 | MI0005789 | >ptr-mir-518c | MI0008714 |
| >dre-mir-27c | MI0001930 | >pta-MIR408 | MI0005790 | >ptr-mir-518d | MI0008715 |
| >dre-mir-27d | MI0001931 | >pta-MIR482a | MI0005791 | >ptr-mir-518e | MI0008716 |
| >dre-mir-27e | MI0001932 | >pta-MIR482b | MI0005792 | >ptr-mir-518f | MI0008717 |
| >dre-mir-29b-1 | MI0001933 | >pta-MIR783 | MI0005793 | >ptr-mir-519a | MI0008718 |
| >dre-mir-29b-2 | MI0001934 | >pta-MIR946a | MI0005794 | >ptr-mir-519b | MI0008719 |
| >dre-mir-29b-3 | MI0001936 | >pta-MIR946b | MI0005795 | >ptr-mir-519c | MI0008720 |
| >dre-mir-29a-1 | MI0001938 | >pta-MIR947 | MI0005796 | >ptr-mir-519d | MI0008721 |
| >dre-mir-30a | MI0001940 | >pta-MIR948 | MI0005797 | >ptr-mir-519e | MI0008722 |
| >dre-mir-30b | MI0001941 | >pta-MIR949 | MI0005798 | >ptr-mir-520a | MI0008723 |
| >dre-mir-30c | MI0001944 | >pta-MIR950a | MI0005799 | >ptr-mir-520b | MI0008724 |
| >dre-mir-30d | MI0001946 | >pta-MIR950b | MI0005800 | >ptr-mir-520c | MI0008725 |
| >dre-mir-30e-2 | MI0001950 | >pta-MIR951 | MI0005801 | >ptr-mir-520d | MI0008726 |
| >dre-mir-92a-1 | MI0001951 | >pta-MIR952a | MI0005802 | >ptr-mir-520e | MI0008727 |
| >dre-mir-92a-2 | MI0001952 | >pta-MIR952b | MI0005803 | >ptr-mir-520f | MI0008728 |
| >dre-mir-92b | MI0001953 | >osa-MIR529b | MI0005804 | >ptr-mir-520g | MI0008729 |
| >dre-mir-93 | MI0001954 | >dme-mir-iab-4as | MI0005805 | >ptr-mir-520h | MI0008730 |
| >dre-mir-96 | MI0001955 | >dme-mir-954 | MI0005806 | >ptr-mir-521-1 | MI0008731 |
| >dre-mir-99-1 | MI0001956 | >dme-mir-955 | MI0005807 | >ptr-mir-521-2 | MI0008732 |
| >dre-mir-99-2 | MI0001957 | >dme-mir-190 | MI0005808 | >ptr-mir-522 | MI0008733 |
| >dre-mir-100-1 | MI0001958 | >dme-mir-193 | MI0005809 | >ptr-mir-523 | MI0008734 |
| >dre-mir-100-2 | MI0001959 | >dme-mir-956 | MI0005810 | >ptr-mir-524 | MI0008735 |
| >dre-mir-101a | MI0001960 | >dme-mir-957 | MI0005811 | >ptr-mir-525 | MI0008736 |
| >dre-mir-101b | MI0001961 | >dme-mir-958 | MI0005812 | >ptr-mir-526a-1 | MI0008737 |
| >dre-mir-103 | MI0001962 | >dme-mir-375 | MI0005813 | >ptr-mir-526a-2 | MI0008738 |
| >dre-mir-107 | MI0001963 | >dme-mir-959 | MI0005814 | >ptr-mir-526b | MI0008739 |
| >dre-mir-122 | MI0001965 | >dme-mir-960 | MI0005815 | >ptr-mir-527 | MI0008740 |
| >dre-mir-124-1 | MI0001966 | >dme-mir-961 | MI0005816 | >ptr-mir-532 | MI0008741 |
| >dre-mir-124-2 | MI0001967 | >dme-mir-962 | MI0005817 | >ptr-mir-539 | MI0008742 |
| >dre-mir-124-3 | MI0001968 | >dme-mir-963 | MI0005818 | >ptr-mir-543 | MI0008743 |
| >dre-mir-124-4 | MI0001969 | >dme-mir-964 | MI0005819 | >ptr-mir-544 | MI0008744 |
| >dre-mir-124-5 | MI0001970 | >dme-mir-932 | MI0005820 | >ptr-mir-545 | MI0008745 |
| >dre-mir-124-6 | MI0001971 | >dme-mir-965 | MI0005821 | >ptr-mir-548a-1 | MI0008746 |
| >dre-mir-125a-1 | MI0001972 | >dme-mir-966 | MI0005822 | >ptr-mir-548a-2 | MI0008747 |
| >dre-mir-125a-2 | MI0001973 | >dme-mir-967 | MI0005823 | >ptr-mir-548b | MI0008748 |
| >dre-mir-125b-1 | MI0001975 | >dme-mir-1002 | MI0005824 | >ptr-mir-548c | MI0008749 |
| >dre-mir-125b-2 | MI0001976 | >dme-mir-968 | MI0005825 | >ptr-mir-548f-1 | MI0008750 |
| >dre-mir-125b-3 | MI0001977 | >dme-mir-969 | MI0005826 | >ptr-mir-548f-2 | MI0008751 |
| >dre-mir-125c | MI0001978 | >dme-mir-970 | MI0005827 | >ptr-mir-548h | MI0008752 |
| >dre-mir-126 | MI0001979 | >dme-mir-971 | MI0005828 | >ptr-mir-548i-1 | MI0008753 |
| >dre-mir-128-1 | MI0001980 | >dme-mir-972 | MI0005829 | >ptr-mir-548i-2 | MI0008754 |
| >dre-mir-128-2 | MI0001981 | >dme-mir-973 | MI0005830 | >ptr-mir-548i-3 | MI0008755 |
| >dre-mir-129-2 | MI0001982 | >dme-mir-974 | MI0005831 | >ptr-mir-548i-4 | MI0008756 |
| >dre-mir-129-1 | MI0001983 | >dme-mir-975 | MI0005832 | >ptr-mir-548i-5 | MI0008757 |
| >dre-mir-130a-1 | MI0001984 | >dme-mir-976 | MI0005833 | >ptr-mir-548i-6 | MI0008758 |
| >dre-mir-130a-2 | MI0001985 | >dme-mir-977 | MI0005834 | >ptr-mir-548j | MI0008759 |
| >dre-mir-130b | MI0001986 | >dme-mir-978 | MI0005835 | >ptr-mir-548k | MI0008760 |
| >dre-mir-130c-1 | MI0001987 | >dme-mir-979 | MI0005836 | >ptr-mir-548l | MI0008761 |
| >dre-mir-130c-2 | MI0001988 | >dme-mir-980 | MI0005837 | >ptr-mir-548n | MI0008762 |
| >dre-mir-132-1 | MI0001989 | >dme-mir-981 | MI0005838 | >ptr-mir-548p | MI0008763 |
| >dre-mir-132-2 | MI0001991 | >dme-mir-982 | MI0005839 | >ptr-mir-549 | MI0008764 |
| >dre-mir-133a-2 | MI0001992 | >dme-mir-983-1 | MI0005840 | >ptr-mir-550-1 | MI0008765 |
| >dre-mir-133a-1 | MI0001993 | >dme-mir-983-2 | MI0005841 | >ptr-mir-550-2 | MI0008766 |
| >dre-mir-133b | MI0001994 | >dme-mir-984 | MI0005842 | >ptr-mir-550-3 | MI0008767 |
| >dre-mir-133c | MI0001995 | >dme-mir-927 | MI0005843 | >ptr-mir-551a | MI0008768 |
| >dre-mir-135c-1 | MI0001996 | >dme-mir-985 | MI0005844 | >ptr-mir-551b | MI0008769 |
| >dre-mir-135c-2 | MI0001997 | >dme-mir-986 | MI0005845 | >ptr-mir-552 | MI0008770 |
| >dre-mir-135c-3 | MI0001998 | >dme-mir-987 | MI0005846 | >ptr-mir-553 | MI0008771 |
| >dre-mir-137-1 | MI0002000 | >dme-mir-988 | MI0005847 | >ptr-mir-554 | MI0008772 |
| >dre-mir-137-2 | MI0002001 | >dme-mir-989 | MI0005848 | >ptr-mir-555 | MI0008773 |
| >dre-mir-138 | MI0002002 | >dme-mir-137 | MI0005849 | >ptr-mir-556 | MI0008774 |
| >dre-mir-140 | MI0002003 | >dme-mir-990 | MI0005850 | >ptr-mir-557 | MI0008775 |
| >dre-mir-141 | MI0002004 | >dme-mir-991 | MI0005851 | >ptr-mir-558 | MI0008776 |
| >dre-mir-142a | MI0002005 | >dme-mir-992 | MI0005852 | >ptr-mir-559 | MI0008777 |
| >dre-mir-142b | MI0002006 | >dme-mir-929 | MI0005853 | >ptr-mir-561 | MI0008778 |
| >dre-mir-143-1 | MI0002007 | >dme-mir-993 | MI0005854 | >ptr-mir-562 | MI0008779 |
| >dre-mir-143-2 | MI0002008 | >dme-mir-994 | MI0005855 | >ptr-mir-564 | MI0008780 |
| >dre-mir-144 | MI0002009 | >dme-mir-995 | MI0005856 | >ptr-mir-566 | MI0008781 |
| >dre-mir-145 | MI0002010 | >dme-mir-996 | MI0005857 | >ptr-mir-567 | MI0008782 |
| >dre-mir-146a | MI0002012 | >dme-mir-252 | MI0005858 | >ptr-mir-568 | MI0008783 |
| >dre-mir-146b-1 | MI0002013 | >dme-mir-997 | MI0005859 | >ptr-mir-569 | MI0008784 |
| >dre-mir-146b-2 | MI0002014 | >dme-mir-998 | MI0005860 | >ptr-mir-572 | MI0008785 |
| >dre-mir-148 | MI0002015 | >dme-mir-999 | MI0005861 | >ptr-mir-575 | MI0008786 |
| >dre-mir-150 | MI0002016 | >dme-mir-1000 | MI0005862 | >ptr-mir-576 | MI0008787 |
| >dre-mir-152 | MI0002017 | >dme-mir-1001 | MI0005863 | >ptr-mir-577 | MI0008788 |
| >dre-mir-153b | MI0002019 | >dme-mir-1003 | MI0005864 | >ptr-mir-579 | MI0008789 |
| >dre-mir-153a | MI0002021 | >dme-mir-1004 | MI0005865 | >ptr-mir-580 | MI0008790 |
| >dre-mir-153c | MI0002022 | >dme-mir-1005 | MI0005866 | >ptr-mir-581 | MI0008791 |
| >dre-mir-155 | MI0002023 | >dme-mir-1006 | MI0005867 | >ptr-mir-582 | MI0008792 |
| >dre-mir-181c | MI0002024 | >dme-mir-1007 | MI0005868 | >ptr-mir-583 | MI0008793 |
| >dre-mir-184 | MI0002026 | >dme-mir-1008 | MI0005869 | >ptr-mir-584 | MI0008794 |
| >dre-mir-190 | MI0002027 | >dme-mir-1009 | MI0005870 | >ptr-mir-586 | MI0008795 |
| >dre-mir-462 | MI0002028 | >dme-mir-1010 | MI0005871 | >ptr-mir-587 | MI0008796 |
| >dre-mir-193a-1 | MI0002029 | >dme-mir-1011 | MI0005872 | >ptr-mir-588 | MI0008797 |
| >dre-mir-193a-2 | MI0002030 | >dme-mir-1012 | MI0005873 | >ptr-mir-590 | MI0008798 |
| >dre-mir-193a-3 | MI0002031 | >dme-mir-1013 | MI0005874 | >ptr-mir-591 | MI0008799 |
| >dre-mir-193b | MI0002032 | >dme-mir-1014 | MI0005875 | >ptr-mir-592 | MI0008800 |
| >dre-mir-194a | MI0002033 | >dme-mir-1015 | MI0005876 | >ptr-mir-593 | MI0008801 |
| >dre-mir-194b | MI0002034 | >dme-mir-1016 | MI0005877 | >ptr-mir-595 | MI0008802 |
| >dre-mir-196a-2 | MI0002035 | >dme-mir-1017 | MI0005878 | >ptr-mir-597 | MI0008803 |
| >dre-mir-196b | MI0002036 | >cel-mir-1018 | MI0005879 | >ptr-mir-598 | MI0008804 |
| >dre-mir-200a | MI0002037 | >cel-mir-1019 | MI0005880 | >ptr-mir-599 | MI0008805 |
| >dre-mir-200b | MI0002038 | >cel-mir-1020 | MI0005881 | >ptr-mir-600 | MI0008806 |
| >dre-mir-200c | MI0002039 | >mdv2-mir-M14 | MI0005882 | >ptr-mir-601 | MI0008807 |
| >dre-mir-202 | MI0002040 | >mdv2-mir-M15 | MI0005883 | >ptr-mir-605 | MI0008808 |
| >dre-mir-203b | MI0002041 | >mdv2-mir-M16 | MI0005884 | >ptr-mir-609 | MI0008809 |
| >dre-mir-204-2 | MI0002042 | >mdv2-mir-M17 | MI0005885 | >ptr-mir-610 | MI0008810 |
| >dre-mir-204-3 | MI0002043 | >mdv2-mir-M18 | MI0005886 | >ptr-mir-612 | MI0008811 |
| >dre-mir-206-1 | MI0002045 | >mdv2-mir-M19 | MI0005887 | >ptr-mir-613 | MI0008812 |
| >dre-mir-206-2 | MI0002046 | >mdv2-mir-M20 | MI0005888 | >ptr-mir-614 | MI0008813 |
| >dre-mir-216a-2 | MI0002047 | >mdv2-mir-M21 | MI0005889 | >ptr-mir-615 | MI0008814 |
| >dre-mir-216b-1 | MI0002048 | >mdv2-mir-M22 | MI0005890 | >ptr-mir-616 | MI0008815 |
| >dre-mir-216b-2 | MI0002049 | >mdv2-mir-M23 | MI0005891 | >ptr-mir-617 | MI0008816 |
| >dre-mir-217-2 | MI0002050 | >mdv2-mir-M24 | MI0005892 | >ptr-mir-618 | MI0008817 |
| >dre-mir-218a-1 | MI0002051 | >mdv2-mir-M25 | MI0005893 | >ptr-mir-619 | MI0008818 |
| >dre-mir-218a-2 | MI0002052 | >mdv2-mir-M26 | MI0005894 | >ptr-mir-621 | MI0008819 |
| >dre-mir-218b | MI0002053 | >mdv2-mir-M27 | MI0005895 | >ptr-mir-622 | MI0008820 |
| >dre-mir-219-3 | MI0002054 | >mdv2-mir-M28 | MI0005896 | >ptr-mir-624 | MI0008821 |
| >dre-mir-301a-1 | MI0002059 | >mdv2-mir-M29 | MI0005897 | >ptr-mir-626 | MI0008822 |
| >dre-mir-301a-2 | MI0002060 | >mdv2-mir-M30 | MI0005898 | >ptr-mir-627 | MI0008823 |
| >dre-mir-301b | MI0002061 | >cel-mir-1021 | MI0005899 | >ptr-mir-628 | MI0008824 |
| >dre-mir-301c | MI0002062 | >cel-mir-1022 | MI0005900 | >ptr-mir-630 | MI0008825 |
| >dre-mir-338-1 | MI0002063 | >ppt-MIR160e | MI0005901 | >ptr-mir-632 | MI0008826 |
| >dre-mir-338-2 | MI0002064 | >ppt-MIR160f | MI0005902 | >ptr-mir-633 | MI0008827 |
| >dre-mir-338-3 | MI0002065 | >ppt-MIR160g | MI0005903 | >ptr-mir-634 | MI0008828 |
| >dre-mir-338-4 | MI0002066 | >ppt-MIR160h | MI0005904 | >ptr-mir-635 | MI0008829 |
| >dre-mir-363 | MI0002067 | >ppt-MIR160i | MI0005905 | >ptr-mir-637 | MI0008830 |
| >dre-mir-365-1 | MI0002068 | >ppt-MIR166c | MI0005906 | >ptr-mir-640 | MI0008831 |
| >dre-mir-365-2 | MI0002069 | >ppt-MIR166d | MI0005907 | >ptr-mir-641 | MI0008832 |
| >dre-mir-365-3 | MI0002070 | >ppt-MIR166e | MI0005908 | >ptr-mir-642 | MI0008833 |
| >dre-mir-365-4 | MI0002071 | >ppt-MIR166f | MI0005909 | >ptr-mir-643 | MI0008834 |
| >dre-mir-375-1 | MI0002072 | >ppt-MIR166g | MI0005910 | >ptr-mir-645 | MI0008835 |
| >dre-mir-375-2 | MI0002073 | >ppt-MIR166h | MI0005911 | >ptr-mir-646 | MI0008836 |
| >dre-mir-454a | MI0002074 | >ppt-MIR166i | MI0005912 | >ptr-mir-649 | MI0008837 |
| >dre-mir-454b | MI0002076 | >ppt-MIR166j | MI0005913 | >ptr-mir-650 | MI0008838 |
| >dre-mir-455 | MI0002077 | >ppt-MIR166k | MI0005914 | >ptr-mir-652 | MI0008839 |
| >dre-mir-430c-2 | MI0002079 | >ppt-MIR166l | MI0005915 | >ptr-mir-653 | MI0008840 |
| >dre-mir-430c-3 | MI0002080 | >ppt-MIR166m | MI0005916 | >ptr-mir-654 | MI0008841 |
| >dre-mir-430c-4 | MI0002081 | >ppt-MIR408b | MI0005917 | >ptr-mir-656 | MI0008842 |
| >dre-mir-430c-5 | MI0002082 | >ppt-MIR477d | MI0005918 | >ptr-mir-657 | MI0008843 |
| >dre-mir-430c-6 | MI0002083 | >ppt-MIR477e | MI0005919 | >ptr-mir-658 | MI0008844 |
| >dre-mir-430c-7 | MI0002087 | >ppt-MIR477h | MI0005920 | >ptr-mir-660 | MI0008845 |
| >dre-mir-430c-8 | MI0002088 | >ppt-MIR529a | MI0005921 | >ptr-mir-663a | MI0008846 |
| >dre-mir-430c-9 | MI0002089 | >ppt-MIR529b | MI0005922 | >ptr-mir-663b | MI0008847 |
| >dre-mir-430c-10 | MI0002090 | >ppt-MIR529c | MI0005923 | >ptr-mir-664 | MI0008848 |
| >dre-mir-430c-11 | MI0002091 | >ppt-MIR529d | MI0005924 | >ptr-mir-665 | MI0008849 |
| >dre-mir-430c-12 | MI0002092 | >ppt-MIR529e | MI0005925 | >ptr-mir-668 | MI0008850 |
| >dre-mir-430c-13 | MI0002093 | >ppt-MIR529f | MI0005926 | >ptr-mir-671 | MI0008851 |
| >dre-mir-430c-14 | MI0002094 | >ppt-MIR529g | MI0005927 | >ptr-mir-708 | MI0008852 |
| >dre-mir-430c-15 | MI0002095 | >ppt-MIR533c | MI0005928 | >ptr-mir-7-1 | MI0008853 |
| >dre-mir-430c-16 | MI0002096 | >ppt-MIR533d | MI0005929 | >ptr-mir-7-2 | MI0008854 |
| >dre-mir-430c-17 | MI0002097 | >ppt-MIR533e | MI0005930 | >ptr-mir-720 | MI0008855 |
| >dre-mir-430c-18 | MI0002098 | >ppt-MIR536d | MI0005932 | >ptr-mir-744 | MI0008856 |
| >dre-mir-430c-19 | MI0002099 | >ppt-MIR536e | MI0005933 | >ptr-mir-758 | MI0008857 |
| >dre-mir-430c-20 | MI0002100 | >ppt-MIR536f | MI0005934 | >ptr-mir-760 | MI0008858 |
| >dre-mir-430c-21 | MI0002101 | >ppt-MIR537c | MI0005935 | >ptr-mir-765 | MI0008859 |
| >dre-mir-430a-2 | MI0002111 | >ppt-MIR537d | MI0005936 | >ptr-mir-766 | MI0008860 |
| >dre-mir-430a-3 | MI0002112 | >ppt-MIR902c | MI0005937 | >ptr-mir-770 | MI0008861 |
| >dre-mir-430a-4 | MI0002113 | >ppt-MIR902d | MI0005938 | >ptr-mir-802 | MI0008862 |
| >dre-mir-430a-5 | MI0002114 | >ppt-MIR902e | MI0005939 | >ptr-mir-873 | MI0008863 |
| >dre-mir-430a-6 | MI0002115 | >ppt-MIR902f | MI0005940 | >ptr-mir-874 | MI0008864 |
| >dre-mir-430a-7 | MI0002116 | >ppt-MIR902g | MI0005941 | >ptr-mir-875 | MI0008865 |
| >dre-mir-430a-8 | MI0002117 | >ppt-MIR902h | MI0005942 | >ptr-mir-876 | MI0008866 |
| >dre-mir-430a-9 | MI0002118 | >ppt-MIR902i | MI0005943 | >ptr-mir-885 | MI0008867 |
| >dre-mir-430a-10 | MI0002119 | >ppt-MIR902j | MI0005944 | >ptr-mir-886 | MI0008868 |
| >dre-mir-430a-11 | MI0002120 | >ppt-MIR902k | MI0005945 | >ptr-mir-887 | MI0008869 |
| >dre-mir-430a-12 | MI0002121 | >ppt-MIR902l | MI0005946 | >ptr-mir-889 | MI0008870 |
| >dre-mir-430a-13 | MI0002122 | >ppt-MIR1222b | MI0005947 | >ptr-mir-890 | MI0008871 |
| >dre-mir-430a-14 | MI0002123 | >ppt-MIR1222c | MI0005948 | >ptr-mir-891a | MI0008872 |
| >dre-mir-430a-15 | MI0002124 | >ppt-MIR1222d | MI0005949 | >ptr-mir-891b | MI0008873 |
| >dre-mir-430a-16 | MI0002125 | >ppt-MIR1222e | MI0005950 | >ptr-mir-892a | MI0008874 |
| >dre-mir-430a-17 | MI0002126 | >ppt-MIR1223b | MI0005951 | >ptr-mir-9-1 | MI0008875 |
| >dre-mir-430a-18 | MI0002131 | >ppt-MIR1223c | MI0005952 | >ptr-mir-920 | MI0008876 |
| >dre-mir-430a-19 | MI0002132 | >ppt-MIR1223d | MI0005953 | >ptr-mir-922 | MI0008877 |
| >dre-mir-430a-20 | MI0002133 | >ppt-MIR1223e | MI0005954 | >ptr-mir-923 | MI0008878 |
| >dre-mir-430a-21 | MI0002134 | >ppt-MIR1223f | MI0005955 | >ptr-mir-924 | MI0008879 |
| >dre-mir-430a-22 | MI0002135 | >ppt-MIR1223g | MI0005956 | >ptr-mir-933 | MI0008880 |
| >dre-mir-430a-23 | MI0002138 | >ppt-MIR1223h | MI0005957 | >ptr-mir-934 | MI0008881 |
| >dre-mir-430i-1 | MI0002139 | >ppt-MIR1223i | MI0005958 | >ptr-mir-935 | MI0008882 |
| >dre-mir-430i-2 | MI0002140 | >ppt-MIR1223j | MI0005959 | >ptr-mir-936 | MI0008883 |
| >dre-mir-430i-3 | MI0002141 | >ppt-MIR1023a | MI0005960 | >ptr-mir-937 | MI0008884 |
| >dre-mir-430b-2 | MI0002142 | >ppt-MIR1023b | MI0005961 | >ptr-mir-938 | MI0008885 |
| >dre-mir-430b-3 | MI0002143 | >ppt-MIR1023c | MI0005962 | >ptr-mir-939 | MI0008886 |
| >dre-mir-430b-4 | MI0002144 | >ppt-MIR1023d | MI0005963 | >ptr-mir-940 | MI0008887 |
| >dre-mir-430b-6 | MI0002146 | >ppt-MIR1024a | MI0005964 | >ptr-mir-942 | MI0008888 |
| >dre-mir-430b-7 | MI0002147 | >ppt-MIR1024b | MI0005965 | >ptr-mir-943 | MI0008889 |
| >dre-mir-430b-8 | MI0002148 | >ppt-MIR1025 | MI0005966 | >ptr-mir-944 | MI0008890 |
| >dre-mir-430b-9 | MI0002149 | >ppt-MIR1026a | MI0005967 | >ptr-mir-99b | MI0008891 |
| >dre-mir-430b-10 | MI0002150 | >ppt-MIR1026b | MI0005968 | >tca-mir-1 | MI0008892 |
| >dre-mir-430b-11 | MI0002151 | >ppt-MIR1027a | MI0005969 | >tca-mir-2-3 | MI0008893 |
| >dre-mir-430b-12 | MI0002152 | >ppt-MIR1027b | MI0005970 | >tca-mir-2-1 | MI0008894 |
| >dre-mir-430b-13 | MI0002153 | >ppt-MIR1028a | MI0005971 | >tca-mir-2-2 | MI0008895 |
| >dre-mir-430b-14 | MI0002154 | >ppt-MIR1028b | MI0005972 | >tca-let-7 | MI0008896 |
| >dre-mir-430b-15 | MI0002155 | >ppt-MIR1028c | MI0005973 | >tca-mir-7 | MI0008897 |
| >dre-mir-430b-16 | MI0002156 | >ppt-MIR1029 | MI0005974 | >tca-mir-8 | MI0008898 |
| >dre-mir-430b-17 | MI0002157 | >ppt-MIR1030a | MI0005975 | >tca-mir-10 | MI0008899 |
| >dre-mir-430b-18 | MI0002158 | >ppt-MIR1030b | MI0005976 | >tca-mir-12 | MI0008900 |
| >dre-mir-430b-5 | MI0002166 | >ppt-MIR1030c | MI0005977 | >tca-mir-13a | MI0008901 |
| >dre-mir-430b-19 | MI0002170 | >ppt-MIR1030d | MI0005978 | >tca-mir-13b | MI0008902 |
| >dre-mir-430b-20 | MI0002171 | >ppt-MIR1030e | MI0005979 | >tca-mir-14 | MI0008903 |
| >dre-mir-430b-21 | MI0002172 | >ppt-MIR1030f | MI0005980 | >tca-mir-31a | MI0008904 |
| >dre-mir-430b-22 | MI0002173 | >ppt-MIR1030g | MI0005981 | >tca-mir-33 | MI0008905 |
| >dre-mir-430b-23 | MI0002174 | >ppt-MIR1030h | MI0005982 | >tca-mir-34 | MI0008906 |
| >dre-mir-430j | MI0002175 | >ppt-MIR1030i | MI0005983 | >tca-mir-71 | MI0008907 |
| >dre-mir-456 | MI0002176 | >ppt-MIR1030j | MI0005984 | >tca-mir-87 | MI0008908 |
| >dre-mir-457a | MI0002177 | >ppt-MIR1031a | MI0005985 | >tca-mir-92a | MI0008909 |
| >dre-mir-457b | MI0002178 | >ppt-MIR1031b | MI0005986 | >tca-mir-92b | MI0008910 |
| >dre-mir-458 | MI0002179 | >ppt-MIR1032 | MI0005987 | >tca-mir-100 | MI0008911 |
| >dre-mir-459 | MI0002180 | >ppt-MIR1033a | MI0005988 | >tca-mir-124 | MI0008912 |
| >dre-mir-460 | MI0002181 | >ppt-MIR1033b | MI0005989 | >tca-mir-125 | MI0008913 |
| >dre-mir-461 | MI0002183 | >ppt-MIR1033c | MI0005990 | >tca-mir-133 | MI0008914 |
| >ptc-MIR156a | MI0002184 | >ppt-MIR1033d | MI0005991 | >tca-mir-137 | MI0008915 |
| >ptc-MIR156b | MI0002185 | >ppt-MIR1033e | MI0005992 | >tca-mir-184 | MI0008916 |
| >ptc-MIR156c | MI0002186 | >ppt-MIR1034 | MI0005993 | >tca-mir-190 | MI0008917 |
| >ptc-MIR156d | MI0002187 | >ppt-MIR1035 | MI0005994 | >tca-mir-210 | MI0008918 |
| >ptc-MIR156e | MI0002188 | >ppt-MIR1036 | MI0005995 | >tca-mir-219 | MI0008919 |
| >ptc-MIR156f | MI0002189 | >ppt-MIR1037 | MI0005996 | >tca-mir-263b | MI0008920 |
| >ptc-MIR156g | MI0002190 | >ppt-MIR1038 | MI0005997 | >tca-mir-275 | MI0008921 |
| >ptc-MIR156h | MI0002191 | >ppt-MIR1039 | MI0005998 | >tca-mir-276 | MI0008922 |
| >ptc-MIR156i | MI0002192 | >ppt-MIR1040 | MI0005999 | >tca-mir-277 | MI0008923 |
| >ptc-MIR156j | MI0002193 | >ppt-MIR1041 | MI0006000 | >tca-mir-279 | MI0008924 |
| >ptc-MIR156k | MI0002194 | >ppt-MIR1042 | MI0006001 | >tca-mir-281 | MI0008925 |
| >ptc-MIR159a | MI0002195 | >ppt-MIR1043 | MI0006002 | >tca-mir-282 | MI0008926 |
| >ptc-MIR159b | MI0002196 | >ppt-MIR1044 | MI0006003 | >tca-mir-283 | MI0008927 |
| >ptc-MIR159c | MI0002197 | >ppt-MIR1045 | MI0006004 | >tca-mir-305 | MI0008928 |
| >ptc-MIR159d | MI0002198 | >ppt-MIR1046 | MI0006005 | >tca-mir-307 | MI0008929 |
| >ptc-MIR159e | MI0002199 | >ppt-MIR1047 | MI0006006 | >tca-mir-317 | MI0008930 |
| >ptc-MIR159f | MI0002200 | >ppt-MIR1048 | MI0006007 | >tca-mir-927 | MI0008931 |
| >ptc-MIR160a | MI0002201 | >ppt-MIR1049 | MI0006008 | >tca-mir-932 | MI0008932 |
| >ptc-MIR160b | MI0002202 | >ppt-MIR1050 | MI0006009 | >tca-mir-iab-4 | MI0008933 |
| >ptc-MIR160c | MI0002203 | >ppt-MIR1051 | MI0006010 | >tca-mir-929 | MI0008934 |
| >ptc-MIR160d | MI0002204 | >ppt-MIR1052 | MI0006011 | >tca-mir-9b | MI0008935 |
| >ptc-MIR160e | MI0002205 | >ppt-MIR1053 | MI0006012 | >tca-mir-9a | MI0008936 |
| >ptc-MIR160f | MI0002206 | >ppt-MIR1054 | MI0006013 | >hsv1-mir-H2 | MI0008937 |
| >ptc-MIR160g | MI0002207 | >ppt-MIR1055 | MI0006014 | >hsv1-mir-H3 | MI0008938 |
| >ptc-MIR160h | MI0002208 | >ppt-MIR1056 | MI0006015 | >hsv1-mir-H4 | MI0008939 |
| >ptc-MIR162a | MI0002209 | >ppt-MIR1057 | MI0006016 | >hsv1-mir-H5 | MI0008940 |
| >ptc-MIR162b | MI0002210 | >ppt-MIR1058 | MI0006017 | >hsv1-mir-H6 | MI0008941 |
| >ptc-MIR162c | MI0002211 | >ppt-MIR1059 | MI0006018 | >dan-mir-92b | MI0008942 |
| >ptc-MIR164a | MI0002212 | >ppt-MIR1060 | MI0006019 | >dan-mir-288 | MI0008943 |
| >ptc-MIR164b | MI0002213 | >ppt-MIR1061 | MI0006020 | >dan-mir-277 | MI0008944 |
| >ptc-MIR164c | MI0002214 | >ppt-MIR1062 | MI0006021 | >dan-mir-8 | MI0008945 |
| >ptc-MIR164d | MI0002215 | >ppt-MIR1063a | MI0006022 | >dan-bantam | MI0008946 |
| >ptc-MIR164e | MI0002216 | >ppt-MIR1063b | MI0006023 | >dan-mir-7 | MI0008947 |
| >ptc-MIR164f | MI0002217 | >ppt-MIR1063c | MI0006024 | >dan-mir-87 | MI0008948 |
| >ptc-MIR166a | MI0002218 | >ppt-MIR1063d | MI0006025 | >dan-mir-92a | MI0008949 |
| >ptc-MIR166b | MI0002219 | >ppt-MIR1063e | MI0006026 | >dan-mir-304 | MI0008950 |
| >ptc-MIR166c | MI0002220 | >ppt-MIR1063f | MI0006027 | >dan-mir-2a-2 | MI0008951 |
| >ptc-MIR166d | MI0002221 | >ppt-MIR1063g | MI0006028 | >dan-mir-284 | MI0008952 |
| >ptc-MIR166e | MI0002222 | >ppt-MIR1063h | MI0006029 | >dan-mir-9b | MI0008953 |
| >ptc-MIR166f | MI0002223 | >ppt-MIR1064 | MI0006030 | >dan-mir-2b | MI0008954 |
| >ptc-MIR166g | MI0002224 | >ppt-MIR1065 | MI0006031 | >dan-mir-12 | MI0008955 |
| >ptc-MIR166h | MI0002225 | >ppt-MIR1066 | MI0006032 | >dan-mir-285 | MI0008956 |
| >ptc-MIR166i | MI0002226 | >ppt-MIR1067 | MI0006033 | >dan-mir-13a | MI0008957 |
| >ptc-MIR166j | MI0002227 | >ppt-MIR1068 | MI0006034 | >dan-mir-308 | MI0008958 |
| >ptc-MIR166k | MI0002228 | >ppt-MIR1069 | MI0006035 | >dan-mir-312 | MI0008959 |
| >ptc-MIR166l | MI0002229 | >ppt-MIR1070 | MI0006036 | >dan-mir-3 | MI0008960 |
| >ptc-MIR166m | MI0002230 | >ppt-MIR1071 | MI0006037 | >dan-mir-287 | MI0008961 |
| >ptc-MIR166n | MI0002231 | >ppt-MIR1072 | MI0006038 | >dan-mir-310 | MI0008962 |
| >ptc-MIR166o | MI0002232 | >ppt-MIR1073 | MI0006039 | >dan-mir-13b-1 | MI0008963 |
| >ptc-MIR166p | MI0002233 | >ppt-MIR1074 | MI0006040 | >dan-mir-4 | MI0008964 |
| >ptc-MIR166q | MI0002234 | >ppt-MIR1075 | MI0006041 | >dan-mir-281-2 | MI0008965 |
| >ptc-MIR167a | MI0002235 | >ppt-MIR1076 | MI0006042 | >dan-mir-79 | MI0008966 |
| >ptc-MIR167b | MI0002236 | >ppt-MIR1077 | MI0006043 | >dan-mir-124 | MI0008967 |
| >ptc-MIR167c | MI0002237 | >ppt-MIR1078 | MI0006044 | >dan-mir-1 | MI0008968 |
| >ptc-MIR167d | MI0002238 | >ppt-MIR1079 | MI0006045 | >dan-mir-210 | MI0008969 |
| >ptc-MIR167e | MI0002239 | >smo-MIR156a | MI0006046 | >dan-mir-307 | MI0008970 |
| >ptc-MIR167f | MI0002240 | >smo-MIR156b | MI0006047 | >dan-mir-184 | MI0008971 |
| >ptc-MIR167g | MI0002241 | >smo-MIR156c | MI0006048 | >dan-mir-283 | MI0008972 |
| >ptc-MIR167h | MI0002242 | >smo-MIR156d | MI0006049 | >dan-mir-280 | MI0008973 |
| >ptc-MIR168a | MI0002243 | >smo-MIR159 | MI0006050 | >dan-mir-34 | MI0008974 |
| >ptc-MIR168b | MI0002244 | >smo-MIR160a | MI0006051 | >dan-mir-iab-4 | MI0008975 |
| >ptc-MIR169a | MI0002245 | >smo-MIR160b | MI0006052 | >dan-mir-2c | MI0008976 |
| >ptc-MIR169aa | MI0002246 | >smo-MIR166a | MI0006053 | >dan-mir-263a | MI0008977 |
| >ptc-MIR169ab | MI0002247 | >smo-MIR166b | MI0006054 | >dan-mir-286 | MI0008978 |
| >ptc-MIR169ac | MI0002248 | >smo-MIR166c | MI0006055 | >dan-mir-282 | MI0008979 |
| >ptc-MIR169ad | MI0002249 | >smo-MIR171a | MI0006056 | >dan-mir-276b | MI0008980 |
| >ptc-MIR169ae | MI0002250 | >smo-MIR171b | MI0006057 | >dan-mir-263b | MI0008981 |
| >ptc-MIR169af | MI0002251 | >smo-MIR171c | MI0006058 | >dan-mir-274 | MI0008982 |
| >ptc-MIR169b | MI0002252 | >smo-MIR171d | MI0006059 | >dan-mir-316 | MI0008983 |
| >ptc-MIR169c | MI0002253 | >smo-MIR396 | MI0006060 | >dan-mir-306 | MI0008984 |
| >ptc-MIR169d | MI0002254 | >smo-MIR408 | MI0006061 | >dan-mir-275 | MI0008985 |
| >ptc-MIR169e | MI0002255 | >smo-MIR536 | MI0006062 | >dan-mir-2a-1 | MI0008986 |
| >ptc-MIR169f | MI0002256 | >smo-MIR1080 | MI0006063 | >dan-mir-281-1 | MI0008987 |
| >ptc-MIR169g | MI0002257 | >smo-MIR1081 | MI0006064 | >dan-mir-317 | MI0008988 |
| >ptc-MIR169h | MI0002258 | >smo-MIR1082a | MI0006065 | >dan-mir-13b-2 | MI0008989 |
| >ptc-MIR169i | MI0002259 | >smo-MIR1082b | MI0006066 | >dan-mir-309 | MI0008990 |
| >ptc-MIR169j | MI0002260 | >smo-MIR1083 | MI0006067 | >dan-mir-279 | MI0008991 |
| >ptc-MIR169k | MI0002261 | >smo-MIR1084 | MI0006068 | >dan-mir-276a | MI0008992 |
| >ptc-MIR169l | MI0002262 | >smo-MIR1085 | MI0006069 | >dan-mir-9c | MI0008993 |
| >ptc-MIR169m | MI0002263 | >smo-MIR1086 | MI0006070 | >dan-mir-5 | MI0008994 |
| >ptc-MIR169n | MI0002264 | >smo-MIR1087 | MI0006071 | >dan-mir-9a | MI0008995 |
| >ptc-MIR169o | MI0002265 | >smo-MIR1088 | MI0006072 | >dan-mir-315 | MI0008996 |
| >ptc-MIR169p | MI0002266 | >smo-MIR1089 | MI0006073 | >dan-mir-14 | MI0008997 |
| >ptc-MIR169q | MI0002267 | >smo-MIR1090 | MI0006074 | >dan-mir-33 | MI0008998 |
| >ptc-MIR169r | MI0002268 | >smo-MIR1091 | MI0006075 | >dan-mir-6-1 | MI0008999 |
| >ptc-MIR169s | MI0002269 | >smo-MIR1092 | MI0006076 | >dan-mir-318 | MI0009000 |
| >ptc-MIR169t | MI0002270 | >smo-MIR1093 | MI0006077 | >dan-mir-305 | MI0009001 |
| >ptc-MIR169u | MI0002271 | >smo-MIR1094a | MI0006078 | >dan-mir-314 | MI0009002 |
| >ptc-MIR169v | MI0002272 | >smo-MIR1094b | MI0006079 | >dan-mir-289 | MI0009003 |
| >ptc-MIR169w | MI0002273 | >smo-MIR1094c | MI0006080 | >dan-let-7 | MI0009004 |
| >ptc-MIR169x | MI0002274 | >smo-MIR1095a | MI0006081 | >dan-mir-133 | MI0009005 |
| >ptc-MIR169y | MI0002275 | >smo-MIR1095b | MI0006082 | >dan-mir-31b | MI0009006 |
| >ptc-MIR169z | MI0002276 | >smo-MIR1096 | MI0006083 | >dan-mir-278 | MI0009007 |
| >ptc-MIR171a | MI0002277 | >smo-MIR1097 | MI0006084 | >dan-mir-311a | MI0009008 |
| >ptc-MIR171b | MI0002278 | >smo-MIR1098 | MI0006085 | >dan-mir-11 | MI0009009 |
| >ptc-MIR171c | MI0002279 | >smo-MIR1099 | MI0006086 | >dan-mir-6-3 | MI0009010 |
| >ptc-MIR171d | MI0002280 | >smo-MIR1100 | MI0006087 | >dan-mir-125 | MI0009011 |
| >ptc-MIR171e | MI0002281 | >smo-MIR1101 | MI0006088 | >dan-mir-219 | MI0009012 |
| >ptc-MIR171f | MI0002282 | >smo-MIR1102 | MI0006089 | >dan-mir-311b | MI0009013 |
| >ptc-MIR171g | MI0002283 | >smo-MIR319 | MI0006090 | >dan-mir-10 | MI0009014 |
| >ptc-MIR171h | MI0002284 | >smo-MIR1104 | MI0006091 | >dan-mir-6-2 | MI0009015 |
| >ptc-MIR171i | MI0002285 | >smo-MIR1105 | MI0006092 | >dan-mir-31a | MI0009016 |
| >ptc-MIR171j | MI0002286 | >smo-MIR1106 | MI0006093 | >dan-mir-100 | MI0009017 |
| >ptc-MIR172a | MI0002287 | >smo-MIR1107 | MI0006094 | >der-mir-308 | MI0009018 |
| >ptc-MIR172b | MI0002288 | >smo-MIR1108 | MI0006095 | >der-mir-283 | MI0009019 |
| >ptc-MIR172c | MI0002289 | >smo-MIR1109 | MI0006096 | >der-mir-31b | MI0009020 |
| >ptc-MIR172d | MI0002290 | >smo-MIR1110 | MI0006097 | >der-mir-5 | MI0009021 |
| >ptc-MIR172e | MI0002291 | >smo-MIR1111 | MI0006098 | >der-mir-287 | MI0009022 |
| >ptc-MIR172f | MI0002292 | >smo-MIR1112 | MI0006099 | >der-mir-13b-2 | MI0009023 |
| >ptc-MIR172g | MI0002293 | >smo-MIR1113 | MI0006100 | >der-mir-310b | MI0009024 |
| >ptc-MIR172h | MI0002294 | >smo-MIR1114 | MI0006101 | >der-let-7 | MI0009025 |
| >ptc-MIR172i | MI0002295 | >smo-MIR1115 | MI0006102 | >der-mir-309 | MI0009026 |
| >ptc-MIR319a | MI0002296 | >smo-MIR1103 | MI0006103 | >der-mir-100 | MI0009027 |
| >ptc-MIR319b | MI0002297 | >hiv1-mir-N367 | MI0006104 | >der-mir-311a | MI0009028 |
| >ptc-MIR319c | MI0002298 | >hiv1-mir-H1 | MI0006106 | >der-mir-133 | MI0009029 |
| >ptc-MIR319d | MI0002299 | >rno-mir-466b-1 | MI0006112 | >der-mir-286 | MI0009030 |
| >ptc-MIR319e | MI0002300 | >rno-mir-466b-2 | MI0006113 | >der-mir-280 | MI0009031 |
| >ptc-MIR319f | MI0002301 | >rno-mir-466c | MI0006114 | >der-mir-92a | MI0009032 |
| >ptc-MIR319g | MI0002302 | >rno-mir-743b | MI0006115 | >der-mir-306 | MI0009033 |
| >ptc-MIR319h | MI0002303 | >rno-mir-871 | MI0006116 | >der-mir-316 | MI0009034 |
| >ptc-MIR319i | MI0002304 | >rno-mir-872 | MI0006117 | >der-mir-307 | MI0009035 |
| >ptc-MIR390a | MI0002305 | >rno-mir-874 | MI0006118 | >der-mir-14 | MI0009036 |
| >ptc-MIR390b | MI0002306 | >rno-mir-877 | MI0006119 | >der-mir-8 | MI0009037 |
| >ptc-MIR390c | MI0002307 | >rno-mir-878 | MI0006120 | >der-mir-11 | MI0009038 |
| >ptc-MIR390d | MI0002308 | >rno-mir-879 | MI0006121 | >der-mir-13b-1 | MI0009039 |
| >ptc-MIR393a | MI0002309 | >rno-mir-880 | MI0006122 | >der-mir-281-2 | MI0009040 |
| >ptc-MIR393b | MI0002310 | >rno-mir-881 | MI0006123 | >der-mir-275 | MI0009041 |
| >ptc-MIR393c | MI0002311 | >rno-mir-883 | MI0006126 | >der-mir-6-3 | MI0009042 |
| >ptc-MIR393d | MI0002312 | >mmu-mir-582 | MI0006127 | >der-mir-9a | MI0009043 |
| >ptc-MIR394a | MI0002313 | >mmu-mir-467e | MI0006128 | >der-mir-263a | MI0009044 |
| >ptc-MIR394b | MI0002314 | >rno-mir-147 | MI0006130 | >der-mir-9b | MI0009045 |
| >ptc-MIR395a | MI0002315 | >rno-mir-17-2 | MI0006131 | >der-mir-34 | MI0009046 |
| >ptc-MIR395b | MI0002316 | >rno-mir-181d | MI0006132 | >der-mir-282 | MI0009047 |
| >ptc-MIR395c | MI0002317 | >rno-mir-182 | MI0006133 | >der-mir-6-2 | MI0009048 |
| >ptc-MIR395d | MI0002318 | >rno-mir-188 | MI0006134 | >der-mir-2a-2 | MI0009049 |
| >ptc-MIR395e | MI0002319 | >rno-mir-190b | MI0006135 | >der-mir-184 | MI0009050 |
| >ptc-MIR395f | MI0002320 | >rno-mir-196c | MI0006136 | >der-mir-6-1 | MI0009051 |
| >ptc-MIR395g | MI0002321 | >rno-mir-301b | MI0006137 | >der-mir-284 | MI0009052 |
| >ptc-MIR395h | MI0002322 | >rno-mir-375 | MI0006140 | >der-mir-285 | MI0009053 |
| >ptc-MIR395i | MI0002323 | >rno-mir-380 | MI0006141 | >der-mir-279 | MI0009054 |
| >ptc-MIR395j | MI0002324 | >rno-mir-384 | MI0006142 | >der-mir-263b | MI0009055 |
| >ptc-MIR396a | MI0002325 | >rno-mir-410 | MI0006143 | >der-mir-277 | MI0009056 |
| >ptc-MIR396b | MI0002326 | >rno-mir-411 | MI0006144 | >der-mir-318 | MI0009057 |
| >ptc-MIR396c | MI0002327 | >rno-mir-423 | MI0006145 | >der-mir-31a | MI0009058 |
| >ptc-MIR396d | MI0002328 | >rno-mir-425 | MI0006146 | >der-mir-2a-1 | MI0009059 |
| >ptc-MIR396e | MI0002329 | >rno-mir-434 | MI0006147 | >der-mir-311b-1 | MI0009060 |
| >ptc-MIR396f | MI0002330 | >rno-mir-455 | MI0006148 | >der-mir-274 | MI0009061 |
| >ptc-MIR396g | MI0002331 | >rno-mir-463 | MI0006149 | >der-mir-311c | MI0009062 |
| >ptc-MIR397a | MI0002332 | >rno-mir-471 | MI0006150 | >der-mir-iab-4 | MI0009063 |
| >ptc-MIR397b | MI0002333 | >rno-mir-484 | MI0006151 | >der-mir-79 | MI0009064 |
| >ptc-MIR397c | MI0002334 | >rno-mir-495 | MI0006152 | >der-mir-311b-2 | MI0009065 |
| >ptc-MIR398a | MI0002335 | >rno-mir-500 | MI0006153 | >der-mir-125 | MI0009066 |
| >ptc-MIR398b | MI0002336 | >rno-mir-532 | MI0006154 | >der-mir-305 | MI0009067 |
| >ptc-MIR398c | MI0002337 | >rno-mir-598 | MI0006155 | >der-mir-281-1 | MI0009068 |
| >ptc-MIR399a | MI0002338 | >rno-mir-671 | MI0006156 | >der-mir-314 | MI0009069 |
| >ptc-MIR399b | MI0002339 | >rno-mir-672 | MI0006157 | >der-mir-276a | MI0009070 |
| >ptc-MIR399c | MI0002340 | >rno-mir-673 | MI0006158 | >der-mir-278 | MI0009071 |
| >ptc-MIR399d | MI0002341 | >rno-mir-674 | MI0006159 | >der-mir-12 | MI0009072 |
| >ptc-MIR399e | MI0002342 | >rno-mir-708 | MI0006160 | >der-mir-4 | MI0009073 |
| >ptc-MIR399f | MI0002343 | >rno-mir-742 | MI0006161 | >der-bantam | MI0009074 |
| >ptc-MIR399g | MI0002344 | >rno-mir-743a | MI0006162 | >der-mir-1 | MI0009075 |
| >ptc-MIR399h | MI0002345 | >rno-mir-758 | MI0006163 | >der-mir-2b-1 | MI0009076 |
| >ptc-MIR399i | MI0002346 | >rno-mir-760 | MI0006164 | >der-mir-312-1 | MI0009077 |
| >ptc-MIR399j | MI0002347 | >rno-mir-770 | MI0006165 | >der-mir-7 | MI0009078 |
| >ptc-MIR399k | MI0002348 | >rno-mir-873 | MI0006166 | >der-mir-3 | MI0009079 |
| >ptc-MIR399l | MI0002349 | >rno-mir-92b | MI0006167 | >der-mir-315 | MI0009080 |
| >ptc-MIR403a | MI0002350 | >rno-mir-488 | MI0006168 | >der-mir-2c | MI0009081 |
| >ptc-MIR403b | MI0002351 | >rno-mir-652 | MI0006169 | >der-mir-317 | MI0009082 |
| >ptc-MIR408 | MI0002352 | >tae-MIR159a | MI0006170 | >der-mir-276b | MI0009083 |
| >ptc-MIR472a | MI0002354 | >tae-MIR159b | MI0006171 | >der-mir-92b | MI0009084 |
| >ptc-MIR472b | MI0002355 | >tae-MIR160 | MI0006172 | >der-mir-87 | MI0009085 |
| >ptc-MIR473a | MI0002356 | >tae-MIR164 | MI0006173 | >der-mir-33 | MI0009086 |
| >ptc-MIR473b | MI0002357 | >tae-MIR167 | MI0006174 | >der-mir-210 | MI0009087 |
| >ptc-MIR474a | MI0002358 | >tae-MIR171 | MI0006175 | >der-mir-124 | MI0009088 |
| >ptc-MIR474b | MI0002359 | >tae-MIR399 | MI0006176 | >der-mir-312-2 | MI0009089 |
| >ptc-MIR474c | MI0002360 | >tae-MIR408 | MI0006177 | >der-mir-304 | MI0009090 |
| >ptc-MIR475a | MI0002361 | >tae-MIR444 | MI0006178 | >der-mir-10 | MI0009091 |
| >ptc-MIR475b | MI0002362 | >tae-MIR1117 | MI0006179 | >der-mir-13a | MI0009092 |
| >ptc-MIR475c | MI0002363 | >tae-MIR1118 | MI0006180 | >der-mir-288 | MI0009093 |
| >ptc-MIR475d | MI0002364 | >tae-MIR1119 | MI0006181 | >der-mir-219 | MI0009094 |
| >ptc-MIR476a | MI0002365 | >tae-MIR1120 | MI0006182 | >der-mir-310a | MI0009095 |
| >ptc-MIR476b | MI0002366 | >tae-MIR1121 | MI0006183 | >der-mir-2b-2 | MI0009096 |
| >ptc-MIR476c | MI0002367 | >tae-MIR1122 | MI0006184 | >der-mir-9c | MI0009097 |
| >ptc-MIR477a | MI0002368 | >tae-MIR1123 | MI0006185 | >der-mir-289 | MI0009098 |
| >ptc-MIR477b | MI0002369 | >tae-MIR1124 | MI0006186 | >dgr-mir-277 | MI0009099 |
| >ptc-MIR478a | MI0002370 | >tae-MIR1125 | MI0006187 | >dgr-mir-100 | MI0009100 |
| >ptc-MIR478b | MI0002371 | >tae-MIR1126 | MI0006188 | >dgr-mir-305 | MI0009101 |
| >ptc-MIR478c | MI0002372 | >tae-MIR1127 | MI0006189 | >dgr-bantam | MI0009102 |
| >ptc-MIR478d | MI0002373 | >tae-MIR1128 | MI0006190 | >dgr-mir-12 | MI0009103 |
| >ptc-MIR478e | MI0002374 | >tae-MIR1129 | MI0006191 | >dgr-mir-219 | MI0009104 |
| >ptc-MIR478f | MI0002375 | >tae-MIR1130 | MI0006192 | >dgr-mir-307 | MI0009105 |
| >ptc-MIR478h | MI0002376 | >tae-MIR1131 | MI0006193 | >dgr-mir-287-1 | MI0009106 |
| >ptc-MIR478i | MI0002377 | >tae-MIR1132 | MI0006194 | >dgr-mir-263b | MI0009107 |
| >ptc-MIR478j | MI0002378 | >tae-MIR1133 | MI0006195 | >dgr-mir-308 | MI0009108 |
| >ptc-MIR478k | MI0002379 | >tae-MIR1134 | MI0006196 | >dgr-mir-184 | MI0009109 |
| >ptc-MIR478l | MI0002380 | >tae-MIR1135 | MI0006197 | >dgr-mir-2b | MI0009110 |
| >ptc-MIR478m | MI0002381 | >tae-MIR1136 | MI0006198 | >dgr-mir-6-2 | MI0009111 |
| >ptc-MIR478n | MI0002382 | >tae-MIR1137 | MI0006199 | >dgr-mir-5-1 | MI0009112 |
| >ptc-MIR478o | MI0002383 | >tae-MIR1138 | MI0006200 | >dgr-mir-92a | MI0009113 |
| >ptc-MIR478p | MI0002384 | >tae-MIR1139 | MI0006201 | >dgr-mir-6-4 | MI0009114 |
| >ptc-MIR478q | MI0002385 | >ppt-MIR477b | MI0006202 | >dgr-mir-11 | MI0009115 |
| >ptc-MIR478r | MI0002386 | >cre-MIR1142 | MI0006203 | >dgr-mir-276b | MI0009116 |
| >ptc-MIR478s | MI0002387 | >cre-MIR1143 | MI0006204 | >dgr-mir-276a | MI0009117 |
| >ptc-MIR478u | MI0002388 | >cre-MIR1144a | MI0006205 | >dgr-mir-309-1 | MI0009118 |
| >ptc-MIR479 | MI0002389 | >cre-MIR1145 | MI0006206 | >dgr-mir-iab-4 | MI0009119 |
| >ptc-MIR480a | MI0002390 | >cre-MIR1146 | MI0006207 | >dgr-mir-3-1 | MI0009120 |
| >ptc-MIR480b | MI0002391 | >cre-MIR1147 | MI0006208 | >dgr-mir-285 | MI0009121 |
| >ptc-MIR481a | MI0002392 | >cre-MIR1148 | MI0006209 | >dgr-mir-9b | MI0009122 |
| >ptc-MIR481b | MI0002393 | >cre-MIR1149 | MI0006210 | >dgr-mir-79 | MI0009123 |
| >ptc-MIR481c | MI0002394 | >cre-MIR1150 | MI0006211 | >dgr-mir-318 | MI0009124 |
| >ptc-MIR481d | MI0002395 | >cre-MIR1151a | MI0006212 | >dgr-mir-31a | MI0009125 |
| >ptc-MIR481e | MI0002396 | >cre-MIR1151b | MI0006213 | >dgr-mir-317 | MI0009126 |
| >ptc-MIR482 | MI0002397 | >cre-MIR1152 | MI0006214 | >dgr-mir-3-2 | MI0009127 |
| >mmu-mir-463 | MI0002398 | >cre-MIR1153 | MI0006215 | >dgr-mir-281-1 | MI0009128 |
| >mmu-mir-464 | MI0002399 | >cre-MIR1154 | MI0006216 | >dgr-mir-9a | MI0009129 |
| >mmu-mir-465a | MI0002400 | >cre-MIR1155 | MI0006217 | >dgr-mir-6-6 | MI0009130 |
| >mmu-mir-466a | MI0002401 | >cre-MIR1156 | MI0006218 | >dgr-mir-286-2 | MI0009131 |
| >mmu-mir-467a | MI0002402 | >cre-MIR1157 | MI0006219 | >dgr-mir-316 | MI0009132 |
| >mmu-mir-468 | MI0002403 | >cre-MIR1158 | MI0006220 | >dgr-mir-306 | MI0009133 |
| >mmu-mir-469 | MI0002404 | >cre-MIR1160 | MI0006221 | >dgr-mir-8 | MI0009134 |
| >mmu-mir-470 | MI0002405 | >cre-MIR1161 | MI0006222 | >dgr-mir-4-2 | MI0009135 |
| >mmu-mir-471 | MI0002406 | >cre-MIR1162 | MI0006223 | >dgr-mir-286-1 | MI0009136 |
| >ath-MIR447a | MI0002407 | >cre-MIR1163 | MI0006224 | >dgr-mir-304 | MI0009137 |
| >ath-MIR447b | MI0002408 | >cre-MIR1164 | MI0006225 | >dgr-let-7-1 | MI0009138 |
| >ath-MIR447c | MI0002409 | >cre-MIR1165 | MI0006226 | >dgr-mir-6-1 | MI0009139 |
| >ssc-mir-105-1 | MI0002410 | >cre-MIR1166 | MI0006227 | >dgr-mir-278 | MI0009140 |
| >ssc-mir-105-2 | MI0002411 | >cre-MIR1168 | MI0006228 | >dgr-mir-314 | MI0009141 |
| >ssc-mir-106a | MI0002412 | >cre-MIR1169 | MI0006229 | >dgr-mir-309-2 | MI0009142 |
| >ssc-mir-122 | MI0002413 | >cre-MIR1170 | MI0006230 | >dgr-mir-279 | MI0009143 |
| >ssc-mir-125b | MI0002414 | >cre-MIR1171 | MI0006231 | >dgr-mir-287-2 | MI0009144 |
| >ssc-mir-135-1 | MI0002415 | >cre-MIR1173 | MI0006232 | >dgr-mir-288 | MI0009145 |
| >ssc-mir-135-2 | MI0002416 | >cre-MIR1172 | MI0006233 | >dgr-mir-10 | MI0009146 |
| >ssc-mir-145 | MI0002417 | >cre-MIR1167 | MI0006234 | >dgr-mir-14 | MI0009147 |
| >ssc-mir-148a | MI0002418 | >cre-MIR1144b | MI0006235 | >dgr-mir-6-5 | MI0009148 |
| >ssc-mir-15b | MI0002419 | >cre-MIR1159 | MI0006236 | >dgr-mir-33 | MI0009149 |
| >ssc-mir-181b | MI0002420 | >aga-mir-1174 | MI0006237 | >dgr-mir-315 | MI0009150 |
| >ssc-mir-184 | MI0002421 | >aga-mir-1175 | MI0006238 | >dgr-mir-13b-1 | MI0009151 |
| >ssc-mir-19a | MI0002422 | >aga-mir-34 | MI0006239 | >dgr-mir-9c | MI0009152 |
| >ssc-mir-20 | MI0002423 | >aga-mir-12 | MI0006240 | >dgr-mir-2a-1 | MI0009153 |
| >ssc-mir-216 | MI0002424 | >aga-mir-996 | MI0006241 | >dgr-mir-87 | MI0009154 |
| >ssc-mir-217 | MI0002425 | >aga-mir-989 | MI0006242 | >dgr-mir-1 | MI0009155 |
| >ssc-mir-224 | MI0002426 | >aga-mir-306 | MI0006243 | >dgr-mir-125-1 | MI0009156 |
| >ssc-mir-23a | MI0002427 | >ddi-mir-1176 | MI0006244 | >dgr-mir-284 | MI0009157 |
| >ssc-mir-24 | MI0002428 | >ddi-mir-1177 | MI0006245 | >dgr-mir-6-3 | MI0009158 |
| >ssc-mir-26a | MI0002429 | >mcmv-mir-m01-1 | MI0006246 | >dgr-mir-4-1 | MI0009159 |
| >ssc-mir-28 | MI0002430 | >mcmv-mir-m01-2 | MI0006247 | >dgr-mir-31b | MI0009160 |
| >ssc-mir-29b | MI0002431 | >mcmv-mir-m01-3 | MI0006248 | >dgr-mir-210 | MI0009161 |
| >ssc-mir-301 | MI0002432 | >mcmv-mir-m01-4 | MI0006249 | >dgr-mir-125-2 | MI0009162 |
| >ssc-mir-323 | MI0002433 | >mcmv-mir-m21-1 | MI0006250 | >dgr-mir-282 | MI0009163 |
| >ssc-mir-326 | MI0002434 | >mcmv-mir-m22-1 | MI0006251 | >dgr-mir-124 | MI0009164 |
| >ssc-mir-7 | MI0002435 | >mcmv-mir-M23-1 | MI0006252 | >dgr-mir-2a-2 | MI0009165 |
| >ssc-mir-95 | MI0002436 | >mcmv-mir-M23-2 | MI0006253 | >dgr-mir-13b-2 | MI0009166 |
| >ssc-mir-140 | MI0002437 | >mcmv-mir-M44-1 | MI0006254 | >dgr-mir-280 | MI0009167 |
| >ssc-mir-181c | MI0002438 | >mcmv-mir-M55-1 | MI0006255 | >dgr-mir-13a | MI0009168 |
| >ssc-mir-183 | MI0002439 | >mcmv-mir-m59-1 | MI0006256 | >dgr-mir-283 | MI0009169 |
| >ssc-mir-205 | MI0002440 | >mcmv-mir-m59-2 | MI0006257 | >dgr-mir-34 | MI0009170 |
| >ssc-mir-214 | MI0002441 | >mcmv-mir-M87-1 | MI0006258 | >dgr-mir-133 | MI0009171 |
| >ssc-mir-27a | MI0002442 | >mcmv-mir-m88-1 | MI0006259 | >dgr-mir-263a | MI0009172 |
| >ssc-mir-32 | MI0002443 | >mcmv-mir-M95-1 | MI0006260 | >dgr-mir-7 | MI0009173 |
| >ssc-mir-325 | MI0002444 | >mcmv-mir-m107-1 | MI0006261 | >dgr-mir-274 | MI0009174 |
| >ssc-let-7c | MI0002445 | >mcmv-mir-m108-1 | MI0006262 | >dgr-mir-5-2 | MI0009175 |
| >ssc-let-7f | MI0002446 | >mcmv-mir-m108-2 | MI0006263 | >dgr-let-7-2 | MI0009176 |
| >ssc-let-7i | MI0002447 | >xtr-mir-31b | MI0006264 | >dgr-mir-281-2 | MI0009177 |
| >ssc-mir-103 | MI0002448 | >xtr-mir-146b | MI0006265 | >dgr-mir-92b | MI0009178 |
| >ssc-mir-107 | MI0002449 | >xtr-mir-320 | MI0006266 | >dgr-mir-275 | MI0009179 |
| >ssc-mir-124a | MI0002450 | >xtr-mir-375 | MI0006267 | >dgr-mir-2c | MI0009180 |
| >ssc-mir-128 | MI0002451 | >xtr-mir-427-1 | MI0006268 | >dmo-mir-316 | MI0009181 |
| >ssc-mir-136 | MI0002452 | >xtr-mir-427-2 | MI0006269 | >dmo-mir-284 | MI0009182 |
| >ssc-mir-139 | MI0002453 | >xtr-mir-427-3 | MI0006270 | >dmo-mir-4 | MI0009183 |
| >ssc-mir-153 | MI0002454 | >hsa-mir-1178 | MI0006271 | >dmo-mir-12 | MI0009184 |
| >ssc-mir-18 | MI0002455 | >hsa-mir-1179 | MI0006272 | >dmo-mir-276b | MI0009185 |
| >ssc-mir-186 | MI0002456 | >hsa-mir-1180 | MI0006273 | >dmo-mir-14 | MI0009186 |
| >ssc-mir-196 | MI0002457 | >hsa-mir-1181 | MI0006274 | >dmo-mir-92a | MI0009187 |
| >ssc-mir-204 | MI0002458 | >hsa-mir-1182 | MI0006275 | >dmo-bantam | MI0009188 |
| >ssc-mir-21 | MI0002459 | >hsa-mir-1183 | MI0006276 | >dmo-mir-6-1 | MI0009189 |
| >ssc-mir-29c | MI0002460 | >hsa-mir-1184 | MI0006277 | >dmo-mir-13b-2 | MI0009190 |
| >ssc-mir-30c | MI0002461 | >mmu-mir-4661 | MI0006278 | >dmo-mir-100 | MI0009191 |
| >ssc-mir-9-1 | MI0002462 | >mmu-mir-669k | MI0006279 | >dmo-mir-219 | MI0009192 |
| >ssc-mir-9-2 | MI0002463 | >mmu-mir-669g | MI0006280 | >dmo-mir-31b | MI0009193 |
| >hsa-mir-412 | MI0002464 | >mmu-mir-669d | MI0006281 | >dmo-mir-283 | MI0009194 |
| >hsa-mir-410 | MI0002465 | >mmu-mir-466i | MI0006282 | >dmo-mir-281-1 | MI0009195 |
| >hsa-mir-376b | MI0002466 | >mmu-mir-1-2-as | MI0006283 | >dmo-mir-286 | MI0009196 |
| >hsa-mir-483 | MI0002467 | >mmu-mir-1186 | MI0006284 | >dmo-mir-314 | MI0009197 |
| >hsa-mir-484 | MI0002468 | >mmu-mir-1187 | MI0006285 | >dmo-mir-282 | MI0009198 |
| >hsa-mir-485 | MI0002469 | >mmu-mir-669j | MI0006286 | >dmo-mir-305 | MI0009199 |
| >hsa-mir-486 | MI0002470 | >mmu-mir-669f | MI0006287 | >dmo-mir-281-2 | MI0009200 |
| >hsa-mir-487a | MI0002471 | >mmu-mir-669i | MI0006288 | >dmo-mir-8 | MI0009201 |
| >kshv-mir-K12-10a | MI0002472 | >mmu-mir-669h | MI0006289 | >dmo-mir-306 | MI0009202 |
| >kshv-mir-K12-10b | MI0002473 | >mmu-mir-1188 | MI0006290 | >dmo-mir-2b | MI0009203 |
| >kshv-mir-K12-11 | MI0002474 | >mmu-mir-466f-4 | MI0006291 | >dmo-mir-285 | MI0009204 |
| >kshv-mir-K12-1 | MI0002475 | >mmu-mir-466k | MI0006292 | >dmo-mir-2c | MI0009205 |
| >kshv-miR-K12-2 | MI0002476 | >mmu-mir-467f | MI0006293 | >dmo-mir-263a | MI0009206 |
| >kshv-mir-K12-9 | MI0002477 | >mmu-mir-1190 | MI0006294 | >dmo-mir-276a | MI0009207 |
| >kshv-mir-K12-8 | MI0002478 | >mmu-mir-466j | MI0006295 | >dmo-mir-274 | MI0009208 |
| >kshv-mir-K12-7 | MI0002479 | >mmu-mir-1191 | MI0006296 | >dmo-mir-34 | MI0009209 |
| >kshv-mir-K12-6 | MI0002480 | >mmu-mir-1192 | MI0006297 | >dmo-mir-304 | MI0009210 |
| >kshv-mir-K12-5 | MI0002481 | >mmu-mir-1193 | MI0006298 | >dmo-mir-92b | MI0009211 |
| >kshv-mir-K12-4 | MI0002482 | >mmu-mir-1194 | MI0006299 | >dmo-mir-287 | MI0009212 |
| >kshv-mir-K12-3 | MI0002483 | >mmu-mir-669e | MI0006300 | >dmo-mir-309 | MI0009213 |
| >mml-mir-200c | MI0002484 | >mmu-mir-467g | MI0006301 | >dmo-mir-13a | MI0009214 |
| >mml-mir-141 | MI0002485 | >mmu-mir-467h | MI0006302 | >dmo-mir-87 | MI0009215 |
| >ggo-mir-200c | MI0002486 | >mmu-mir-1195 | MI0006303 | >dmo-mir-280 | MI0009216 |
| >ggo-mir-141 | MI0002487 | >mmu-mir-1196 | MI0006304 | >dmo-mir-275 | MI0009217 |
| >ppy-mir-200c | MI0002488 | >mmu-mir-1197 | MI0006305 | >dmo-mir-307 | MI0009218 |
| >ppy-mir-141 | MI0002489 | >mmu-mir-1198 | MI0006306 | >dmo-mir-9b | MI0009219 |
| >ppa-mir-141 | MI0002490 | >mmu-mir-1199 | MI0006307 | >dmo-mir-210 | MI0009220 |
| >ggo-mir-15b | MI0002491 | >cpa-MIR162a | MI0006308 | >dmo-mir-3 | MI0009221 |
| >age-mir-15b | MI0002492 | >ptr-mir-1224 | MI0006309 | >dmo-mir-125 | MI0009222 |
| >ppa-mir-15b | MI0002493 | >mml-mir-1224 | MI0006310 | >dmo-mir-9a | MI0009223 |
| >ppy-mir-15b | MI0002494 | >hsa-mir-1225 | MI0006311 | >dmo-mir-317 | MI0009224 |
| >ptr-mir-15b | MI0002495 | >mml-mir-1225 | MI0006312 | >dmo-mir-2a-2 | MI0009225 |
| >mml-mir-15b | MI0002496 | >hsa-mir-1226 | MI0006313 | >dmo-mir-6-3 | MI0009226 |
| >lla-mir-15b | MI0002497 | >ptr-mir-1226 | MI0006314 | >dmo-mir-184 | MI0009227 |
| >mne-mir-15b | MI0002498 | >mml-mir-1226 | MI0006315 | >dmo-mir-288 | MI0009228 |
| >ptr-mir-23b | MI0002499 | >hsa-mir-1227 | MI0006316 | >dmo-mir-278 | MI0009229 |
| >ppy-mir-23b | MI0002500 | >mml-mir-1227 | MI0006317 | >dmo-mir-31a | MI0009230 |
| >ppa-mir-23b | MI0002501 | >hsa-mir-1228 | MI0006318 | >dmo-mir-133 | MI0009231 |
| >mml-mir-30b | MI0002502 | >hsa-mir-1229 | MI0006319 | >dmo-mir-6-2 | MI0009232 |
| >ptr-mir-30b | MI0002503 | >mml-mir-1230 | MI0006320 | >dmo-mir-11 | MI0009233 |
| >ggo-mir-30b | MI0002504 | >hsa-mir-1231 | MI0006321 | >dmo-mir-iab-4 | MI0009234 |
| >lla-mir-30b | MI0002505 | >mml-mir-1232 | MI0006322 | >dmo-mir-279 | MI0009235 |
| >mne-mir-30b | MI0002506 | >hsa-mir-1233 | MI0006323 | >dmo-mir-9c | MI0009236 |
| >age-mir-30b | MI0002507 | >hsa-mir-1234 | MI0006324 | >dmo-mir-318 | MI0009237 |
| >ppa-mir-30b | MI0002508 | >mml-mir-1235 | MI0006325 | >dmo-mir-277 | MI0009238 |
| >ggo-mir-125b-1 | MI0002509 | >hsa-mir-1236 | MI0006326 | >dmo-let-7 | MI0009239 |
| >age-mir-125b-1 | MI0002510 | >hsa-mir-1237 | MI0006327 | >dmo-mir-315 | MI0009240 |
| >ppa-mir-125b | MI0002511 | >hsa-mir-1238 | MI0006328 | >dmo-mir-124 | MI0009241 |
| >ppy-mir-125b-1 | MI0002512 | >mml-mir-1239 | MI0006329 | >dmo-mir-2a-1 | MI0009242 |
| >ptr-mir-125b-1 | MI0002513 | >mml-mir-1240 | MI0006330 | >dmo-mir-308 | MI0009243 |
| >mml-mir-125b-1 | MI0002514 | >mml-mir-1241 | MI0006331 | >dmo-mir-79 | MI0009244 |
| >sla-mir-125b | MI0002515 | >hsa-mir-1200 | MI0006332 | >dmo-mir-5 | MI0009245 |
| >lla-mir-125b-1 | MI0002516 | >hsa-mir-1201 | MI0006333 | >dmo-mir-7 | MI0009246 |
| >mne-mir-125b-1 | MI0002517 | >hsa-mir-1202 | MI0006334 | >dmo-mir-10 | MI0009247 |
| >mml-mir-128a | MI0002518 | >hsa-mir-1203 | MI0006335 | >dmo-mir-1 | MI0009248 |
| >ptr-mir-128-1 | MI0002519 | >hsa-mir-663b | MI0006336 | >dmo-mir-33 | MI0009249 |
| >ppy-mir-128 | MI0002520 | >hsa-mir-1204 | MI0006337 | >dmo-mir-313 | MI0009250 |
| >sla-mir-128 | MI0002521 | >hsa-mir-1205 | MI0006338 | >dmo-mir-13b-1 | MI0009251 |
| >age-mir-128 | MI0002522 | >hsa-mir-1206 | MI0006339 | >dpe-mir-307 | MI0009252 |
| >ppa-mir-128 | MI0002523 | >hsa-mir-1207 | MI0006340 | >dpe-mir-3 | MI0009253 |
| >mml-mir-130a | MI0002524 | >hsa-mir-1208 | MI0006341 | >dpe-let-7-1 | MI0009254 |
| >ggo-mir-130a | MI0002525 | >rno-mir-146b | MI0006342 | >dpe-mir-184 | MI0009255 |
| >mne-mir-130a | MI0002526 | >rno-mir-551b | MI0006343 | >dpe-mir-277 | MI0009256 |
| >ppa-mir-130a | MI0002527 | >hsa-mir-548e | MI0006344 | >dpe-mir-14 | MI0009257 |
| >ggo-mir-133a | MI0002528 | >hsa-mir-548j | MI0006345 | >dpe-mir-210 | MI0009258 |
| >age-mir-133a | MI0002529 | >hsa-mir-1285-1 | MI0006346 | >dpe-mir-276a | MI0009259 |
| >ppa-mir-133a | MI0002530 | >hsa-mir-1285-2 | MI0006347 | >dpe-mir-13a | MI0009260 |
| >ppy-mir-133a | MI0002531 | >hsa-mir-1286 | MI0006348 | >dpe-mir-6-3 | MI0009261 |
| >ptr-mir-133a-1 | MI0002532 | >hsa-mir-1287 | MI0006349 | >dpe-mir-284 | MI0009262 |
| >mml-mir-133a | MI0002533 | >hsa-mir-1289-1 | MI0006350 | >dpe-mir-286 | MI0009263 |
| >sla-mir-133a | MI0002534 | >hsa-mir-1289-2 | MI0006351 | >dpe-mir-8 | MI0009264 |
| >lla-mir-133a | MI0002535 | >hsa-mir-1290 | MI0006352 | >dpe-mir-13b-2 | MI0009265 |
| >mne-mir-133a | MI0002536 | >hsa-mir-1291 | MI0006353 | >dpe-mir-133 | MI0009266 |
| >lla-mir-135-1 | MI0002537 | >hsa-mir-548k | MI0006354 | >dpe-mir-283 | MI0009267 |
| >age-mir-135-1 | MI0002538 | >hsa-mir-1293 | MI0006355 | >dpe-mir-5 | MI0009268 |
| >ppa-mir-135-1 | MI0002539 | >hsa-mir-1294 | MI0006356 | >dpe-mir-34 | MI0009269 |
| >mml-mir-135a-2 | MI0002540 | >hsa-mir-1295 | MI0006357 | >dpe-mir-282 | MI0009270 |
| >ptr-mir-135a-2 | MI0002541 | >hsa-mir-1297 | MI0006358 | >dpe-mir-9a | MI0009271 |
| >ggo-mir-135 | MI0002542 | >hsa-mir-1299 | MI0006359 | >dpe-mir-100-2 | MI0009272 |
| >ppy-mir-135 | MI0002543 | >hsa-mir-1300 | MI0006360 | >dpe-mir-219 | MI0009273 |
| >lla-mir-135-2 | MI0002544 | >hsa-mir-5481 | MI0006361 | >dpe-mir-314 | MI0009274 |
| >age-mir-135-2 | MI0002545 | >hsa-mir-1302-1 | MI0006362 | >dpe-bantam | MI0009275 |
| >ppa-mir-135-2 | MI0002546 | >hsa-mir-1302-2 | MI0006363 | >dpe-mir-280 | MI0009276 |
| >ptr-mir-140 | MI0002547 | >hsa-mir-1302-3 | MI0006364 | >dpe-mir-31a | MI0009277 |
| >mne-mir-140 | MI0002548 | >hsa-mir-1302-4 | MI0006365 | >dpe-mir-1 | MI0009278 |
| >ptr-mir-143 | MI0002549 | >hsa-mir-1302-5 | MI0006366 | >dpe-mir-125-2 | MI0009279 |
| >ggo-mir-143 | MI0002550 | >hsa-mir-1302-6 | MI0006367 | >dpe-mir-278 | MI0009280 |
| >ppy-mir-143 | MI0002551 | >hsa-mir-1302-7 | MI0006368 | >dpe-mir-281-1 | MI0009281 |
| >lla-mir-143 | MI0002552 | >hsa-mir-1302-8 | MI0006369 | >dpe-mir-2a-2 | MI0009282 |
| >ppa-mir-143 | MI0002553 | >hsa-mir-1303 | MI0006370 | >dpe-mir-315 | MI0009283 |
| >ptr-mir-144 | MI0002554 | >hsa-mir-1304 | MI0006371 | >dpe-mir-124 | MI0009284 |
| >ppy-mir-144 | MI0002555 | >hsa-mir-1305 | MI0006372 | >dpe-mir-308 | MI0009285 |
| >mne-mir-144 | MI0002556 | >hsa-mir-1243 | MI0006373 | >dpe-mir-125-1 | MI0009286 |
| >ppa-mir-144 | MI0002557 | >hsa-mir-548f-1 | MI0006374 | >dpe-mir-87 | MI0009287 |
| >mml-mir-145 | MI0002558 | >hsa-mir-548f-2 | MI0006375 | >dpe-mir-279 | MI0009288 |
| >ptr-mir-145 | MI0002559 | >hsa-mir-548f-3 | MI0006376 | >dpe-mir-263b | MI0009289 |
| >ggo-mir-145 | MI0002560 | >hsa-mir-548f-4 | MI0006377 | >dpe-mir-2c | MI0009290 |
| >ppy-mir-145 | MI0002561 | >hsa-mir-548f-5 | MI0006378 | >dpe-mir-13b-1 | MI0009291 |
| >mne-mir-145 | MI0002562 | >hsa-mir-1244 | MI0006379 | >dpe-mir-11 | MI0009292 |
| >mml-mir-153-1 | MI0002563 | >hsa-mir-1245 | MI0006380 | >dpe-mir-2b | MI0009293 |
| >ppy-mir-153 | MI0002564 | >hsa-mir-1246 | MI0006381 | >dpe-mir-317-2 | MI0009294 |
| >mne-mir-153-1 | MI0002565 | >hsa-mir-1247 | MI0006382 | >dpe-mir-125-3 | MI0009295 |
| >mml-mir-153-2 | MI0002566 | >hsa-mir-1248 | MI0006383 | >dpe-mir-iab-4 | MI0009296 |
| >ggo-mir-153 | MI0002567 | >hsa-mir-1249 | MI0006384 | >dpe-mir-7 | MI0009297 |
| >mne-mir-153-2 | MI0002568 | >hsa-mir-1250 | MI0006385 | >dpe-mir-2a-1 | MI0009298 |
| >ptr-mir-9-2 | MI0002569 | >hsa-mir-1251 | MI0006386 | >dpe-mir-33 | MI0009299 |
| >ggo-mir-9 | MI0002570 | >hsa-mir-1253 | MI0006387 | >dpe-mir-275 | MI0009300 |
| >lla-mir-9 | MI0002571 | >hsa-mir-1254 | MI0006388 | >dpe-mir-9b | MI0009301 |
| >mne-mir-9 | MI0002572 | >hsa-mir-1255a | MI0006389 | >dpe-mir-317-1 | MI0009302 |
| >age-mir-9 | MI0002573 | >hsa-mir-1256 | MI0006390 | >dpe-mir-9c | MI0009303 |
| >ggo-mir-125b-2 | MI0002574 | >hsa-mir-1257 | MI0006391 | >dpe-mir-79 | MI0009304 |
| >age-mir-125b-2 | MI0002575 | >hsa-mir-1258 | MI0006392 | >dpe-mir-289 | MI0009305 |
| >lca-mir-125b | MI0002576 | >hsa-mir-1259 | MI0006393 | >dpe-mir-318 | MI0009306 |
| >ppy-mir-125b-2 | MI0002577 | >hsa-mir-1260 | MI0006394 | >dpe-mir-276b | MI0009307 |
| >ptr-mir-125b-2 | MI0002578 | >hsa-mir-548g | MI0006395 | >dpe-mir-4 | MI0009308 |
| >mml-mir-125b-2 | MI0002579 | >hsa-mir-1261 | MI0006396 | >dpe-mir-31b | MI0009309 |
| >lla-mir-125b-2 | MI0002580 | >hsa-mir-1262 | MI0006397 | >dpe-mir-288 | MI0009310 |
| >mne-mir-125b-2 | MI0002581 | >hsa-mir-1263 | MI0006398 | >dpe-mir-10 | MI0009311 |
| >mml-mir-127 | MI0002582 | >hsa-mir-548n | MI0006399 | >dpe-mir-281-2 | MI0009312 |
| >ptr-mir-127 | MI0002583 | >hsa-mir-548m | MI0006400 | >dpe-mir-305 | MI0009313 |
| >ppy-mir-127 | MI0002584 | >hsa-mir-1265 | MI0006401 | >dpe-mir-92b | MI0009314 |
| >sla-mir-127 | MI0002585 | >hsa-mir-548o | MI0006402 | >dpe-mir-287 | MI0009315 |
| >lla-mir-127 | MI0002586 | >hsa-mir-1266 | MI0006403 | >dpe-let-7-2 | MI0009316 |
| >mne-mir-127 | MI0002587 | >hsa-mir-1267 | MI0006404 | >dpe-mir-6-1 | MI0009317 |
| >age-mir-127 | MI0002588 | >hsa-mir-1268 | MI0006405 | >dpe-mir-274 | MI0009318 |
| >ggo-mir-134 | MI0002589 | >hsa-mir-1269 | MI0006406 | >dpe-mir-306 | MI0009319 |
| >ppy-mir-134 | MI0002590 | >hsa-mir-1270 | MI0006407 | >dpe-mir-309 | MI0009320 |
| >mne-mir-134 | MI0002591 | >hsa-mir-1272 | MI0006408 | >dpe-mir-263a | MI0009321 |
| >ppa-mir-134 | MI0002592 | >hsa-mir-1273 | MI0006409 | >dpe-mir-6-2 | MI0009322 |
| >ptr-mir-136 | MI0002593 | >hsa-mir-1274a | MI0006410 | >dpe-mir-12 | MI0009323 |
| >ggo-mir-136 | MI0002594 | >hsa-mir-548h-1 | MI0006411 | >dpe-mir-100-1 | MI0009324 |
| >ppy-mir-136 | MI0002595 | >hsa-mir-548h-2 | MI0006412 | >dpe-mir-92a | MI0009325 |
| >ppa-mir-136 | MI0002596 | >hsa-mir-548h-3 | MI0006413 | >dpe-mir-316 | MI0009326 |
| >ptr-mir-154 | MI0002597 | >hsa-mir-548h-4 | MI0006414 | >dse-mir-6-1 | MI0009327 |
| >ggo-mir-154 | MI0002598 | >hsa-mir-1275 | MI0006415 | >dse-mir-12 | MI0009328 |
| >ppy-mir-154 | MI0002599 | >hsa-mir-1276 | MI0006416 | >dse-mir-280 | MI0009329 |
| >mne-mir-154 | MI0002600 | >hsa-mir-302e | MI0006417 | >dse-mir-263a | MI0009330 |
| >ppa-mir-154 | MI0002601 | >hsa-mir-302f | MI0006418 | >dse-mir-79 | MI0009331 |
| >ptr-mir-184 | MI0002602 | >hsa-mir-1277 | MI0006419 | >dse-mir-287 | MI0009332 |
| >ppy-mir-184 | MI0002603 | >hsa-mir-548p | MI0006420 | >dse-mir-7 | MI0009333 |
| >mne-mir-184 | MI0002604 | >hsa-mir-548i-1 | MI0006421 | >dse-mir-8 | MI0009334 |
| >ptr-mir-186 | MI0002605 | >hsa-mir-548i-2 | MI0006422 | >dse-mir-87 | MI0009335 |
| >ggo-mir-186 | MI0002606 | >hsa-mir-548i-3 | MI0006423 | >dse-mir-92b | MI0009336 |
| >ppa-mir-186 | MI0002607 | >hsa-mir-548i-4 | MI0006424 | >dse-bantam | MI0009337 |
| >mml-mir-188 | MI0002608 | >hsa-mir-1278 | MI0006425 | >dse-mir-210 | MI0009338 |
| >ptr-mir-188 | MI0002609 | >hsa-mir-1279 | MI0006426 | >dse-mir-2c | MI0009339 |
| >ppy-mir-188 | MI0002610 | >hsa-mir-1274b | MI0006427 | >dse-mir-276b | MI0009340 |
| >mne-mir-188 | MI0002611 | >hsa-mir-1281 | MI0006428 | >dse-mir-310 | MI0009341 |
| >ppa-mir-188 | MI0002612 | >hsa-mir-1282 | MI0006429 | >dse-mir-2b-1 | MI0009342 |
| >mml-mir-190a | MI0002613 | >hsa-mir-1283-2 | MI0006430 | >dse-mir-278 | MI0009343 |
| >ptr-mir-190a | MI0002614 | >hsa-mir-1284 | MI0006431 | >dse-mir-281-2 | MI0009344 |
| >ggo-mir-190 | MI0002615 | >hsa-mir-1288 | MI0006432 | >dse-mir-2b-2 | MI0009345 |
| >ppa-mir-190 | MI0002616 | >hsa-mir-1292 | MI0006433 | >dse-mir-10 | MI0009346 |
| >ggo-mir-195 | MI0002617 | >hsa-mir-1252 | MI0006434 | >dse-mir-92a | MI0009347 |
| >ppa-mir-195 | MI0002618 | >hsa-mir-1255b-1 | MI0006435 | >dse-mir-13a | MI0009348 |
| >ppy-mir-206 | MI0002619 | >hsa-mir-1255b-2 | MI0006436 | >dse-mir-34 | MI0009349 |
| >mne-mir-206 | MI0002620 | >hsa-mir-1280 | MI0006437 | >dse-mir-11 | MI0009350 |
| >mml-mir-21 | MI0002621 | >bol-MIR824 | MI0006438 | >dse-mir-317 | MI0009351 |
| >ptr-mir-21 | MI0002622 | >bra-MIR824 | MI0006439 | >dse-mir-305 | MI0009352 |
| >ggo-mir-21 | MI0002623 | >bna-MIR824 | MI0006440 | >dse-mir-276a | MI0009353 |
| >ppy-mir-21 | MI0002624 | >hsa-mir-1308 | MI0006441 | >dse-mir-282 | MI0009354 |
| >mne-mir-21 | MI0002625 | >hsa-mir-664 | MI0006442 | >dse-mir-308 | MI0009355 |
| >age-mir-21 | MI0002626 | >hsa-mir-1306 | MI0006443 | >dse-mir-277 | MI0009356 |
| >ppa-mir-21 | MI0002627 | >hsa-mir-1307 | MI0006444 | >dse-mir-314 | MI0009357 |
| >age-mir-22 | MI0002628 | >bna-MIR397a | MI0006445 | >dse-mir-100 | MI0009358 |
| >ppa-mir-22 | MI0002629 | >bna-MIR397b | MI0006446 | >dse-mir-286 | MI0009359 |
| >lca-mir-22 | MI0002630 | >bna-MIR390a | MI0006447 | >dse-mir-284 | MI0009360 |
| >mml-mir-22 | MI0002631 | >bna-MIR390b | MI0006448 | >dse-mir-311 | MI0009361 |
| >ppy-mir-22 | MI0002632 | >bna-MIR390c | MI0006449 | >dse-mir-9a | MI0009362 |
| >ptr-mir-22 | MI0002633 | >bna-MIR171a | MI0006450 | >dse-mir-304 | MI0009363 |
| >sla-mir-22 | MI0002634 | >bna-MIR171b | MI0006451 | >dse-mir-33 | MI0009364 |
| >lla-mir-22 | MI0002635 | >bna-MIR171c | MI0006452 | >dse-mir-281-1 | MI0009365 |
| >mne-mir-22 | MI0002636 | >bna-MIR171d | MI0006453 | >dse-mir-309 | MI0009366 |
| >mml-mir-24-1 | MI0002637 | >bna-MIR171e | MI0006454 | >dse-mir-306 | MI0009367 |
| >ppy-mir-24-1 | MI0002638 | >bna-MIR171f | MI0006455 | >dse-mir-288 | MI0009368 |
| >mne-mir-24-1 | MI0002639 | >bna-MIR169a | MI0006457 | >dse-mir-289 | MI0009369 |
| >ppa-mir-24-1 | MI0002640 | >bna-MIR169b | MI0006458 | >dse-mir-279 | MI0009370 |
| >ptr-mir-26a-1 | MI0002641 | >bna-MIR169c | MI0006459 | >dse-mir-285 | MI0009371 |
| >ggo-mir-26a | MI0002642 | >bna-MIR169d | MI0006460 | >dse-mir-312 | MI0009372 |
| >ppy-mir-26a | MI0002643 | >bna-MIR169e | MI0006461 | >dse-mir-307 | MI0009373 |
| >lla-mir-26a | MI0002644 | >bna-MIR169f | MI0006462 | >dse-mir-9c | MI0009374 |
| >mne-mir-26a | MI0002645 | >bna-MIR169g | MI0006463 | >dse-mir-14 | MI0009375 |
| >mml-mir-26a-1 | MI0002646 | >bna-MIR169h | MI0006464 | >dse-mir-1 | MI0009376 |
| >ppa-mir-26a | MI0002647 | >bna-MIR169i | MI0006465 | >dse-mir-315 | MI0009377 |
| >age-mir-28 | MI0002648 | >bna-MIR169j | MI0006466 | >dse-mir-13b-2 | MI0009378 |
| >mml-mir-28 | MI0002649 | >bna-MIR169k | MI0006467 | >dse-mir-318 | MI0009379 |
| >ptr-mir-28 | MI0002650 | >bna-MIR169l | MI0006468 | >dse-mir-13b-1 | MI0009380 |
| >ggo-mir-28 | MI0002651 | >bna-MIR169m | MI0006469 | >dse-mir-316 | MI0009381 |
| >ppy-mir-28 | MI0002652 | >bna-MIR168 | MI0006470 | >dse-mir-2a-3 | MI0009382 |
| >mne-mir-28 | MI0002653 | >bna-MIR167a | MI0006471 | >dse-mir-275 | MI0009383 |
| >sla-mir-28 | MI0002654 | >bna-MIR167b | MI0006472 | >dse-mir-184 | MI0009384 |
| >lla-mir-28 | MI0002655 | >bna-MIR167c | MI0006473 | >dse-mir-31b | MI0009385 |
| >ppa-mir-28 | MI0002656 | >bna-MIR166a | MI0006474 | >dse-mir-219 | MI0009386 |
| >ggo-mir-29a | MI0002657 | >bna-MIR166b | MI0006475 | >dse-mir-31a | MI0009387 |
| >age-mir-29a | MI0002658 | >bna-MIR166c | MI0006476 | >dse-mir-125 | MI0009388 |
| >ppa-mir-29a | MI0002659 | >bna-MIR166d | MI0006477 | >dse-mir-9b | MI0009389 |
| >ppy-mir-29a | MI0002660 | >bna-MIR164 | MI0006478 | >dse-mir-274 | MI0009390 |
| >ptr-mir-29a | MI0002661 | >bna-MIR161 | MI0006479 | >dse-mir-5 | MI0009391 |
| >mml-mir-29a | MI0002662 | >bna-MIR159 | MI0006480 | >dse-mir-2a-2 | MI0009392 |
| >sla-mir-29a | MI0002663 | >bna-MIR156b | MI0006481 | >dse-mir-263b | MI0009393 |
| >lla-mir-29a | MI0002664 | >bna-MIR1140a | MI0006482 | >dse-mir-313 | MI0009394 |
| >mne-mir-29a | MI0002665 | >bna-MIR1140b | MI0006483 | >dse-mir-iab-4 | MI0009395 |
| >mml-mir-30a | MI0002666 | >bna-MIR156c | MI0006484 | >dse-mir-133 | MI0009396 |
| >ptr-mir-30a | MI0002667 | >vvi-MIR156a | MI0006485 | >dse-mir-2a-1 | MI0009397 |
| >ggo-mir-30a | MI0002668 | >vvi-MIR156b | MI0006486 | >dse-mir-4 | MI0009398 |
| >ppy-mir-30a | MI0002669 | >vvi-MIR156c | MI0006487 | >dse-mir-3 | MI0009399 |
| >ppa-mir-30a | MI0002670 | >vvi-MIR156d | MI0006488 | >dse-let-7 | MI0009400 |
| >mml-mir-31 | MI0002671 | >vvi-MIR156e | MI0006489 | >dse-mir-283 | MI0009401 |
| >ptr-mir-31 | MI0002672 | >vvi-MIR156f | MI0006490 | >dse-mir-124 | MI0009402 |
| >ggo-mir-31 | MI0002673 | >vvi-MIR156g | MI0006491 | >dse-mir-6-2 | MI0009403 |
| >ppy-mir-31 | MI0002674 | >vvi-MIR156i | MI0006492 | >dse-mir-6-3 | MI0009404 |
| >mne-mir-31 | MI0002675 | >vvi-MIR159a | MI0006493 | >dsi-mir-34 | MI0009405 |
| >ppa-mir-31 | MI0002676 | >vvi-MIR159b | MI0006494 | >dsi-mir-310 | MI0009406 |
| >mml-mir-32 | MI0002677 | >vvi-MIR159c | MI0006495 | >dsi-mir-4 | MI0009407 |
| >ptr-mir-32 | MI0002678 | >vvi-MIR160a | MI0006496 | >dsi-mir-9a | MI0009408 |
| >ggo-mir-32 | MI0002679 | >vvi-MIR160b | MI0006497 | >dsi-mir-87 | MI0009409 |
| >ppy-mir-32 | MI0002680 | >vvi-MIR160c | MI0006498 | >dsi-mir-13b-2 | MI0009410 |
| >sla-mir-32 | MI0002681 | >vvi-MIR160d | MI0006499 | >dsi-mir-125 | MI0009411 |
| >mne-mir-32 | MI0002682 | >vvi-MIR160e | MI0006500 | >dsi-mir-124 | MI0009412 |
| >ppa-mir-32 | MI0002683 | >vvi-MIR160f | MI0006501 | >dsi-mir-14 | MI0009413 |
| >mml-mir-33a | MI0002684 | >vvi-MIR162 | MI0006502 | >dsi-mir-210 | MI0009414 |
| >ptr-mir-33a | MI0002685 | >vvi-MIR164a | MI0006503 | >dsi-mir-277 | MI0009415 |
| >ggo-mir-33 | MI0002686 | >vvi-MIR164b | MI0006504 | >dsi-mir-263a | MI0009416 |
| >ppy-mir-33 | MI0002687 | >vvi-MIR164c | MI0006505 | >dsi-mir-12 | MI0009417 |
| >mne-mir-33 | MI0002688 | >vvi-MIR164d | MI0006506 | >dsi-mir-2a-1 | MI0009418 |
| >ppa-mir-33 | MI0002689 | >vvi-MIR166a | MI0006507 | >dsi-mir-2c | MI0009419 |
| >ptr-mir-95 | MI0002690 | >vvi-MIR166b | MI0006508 | >dsi-mir-iab-4 | MI0009420 |
| >ggo-mir-95 | MI0002691 | >vvi-MIR166c | MI0006509 | >dsi-mir-13b-1 | MI0009421 |
| >ppy-mir-95 | MI0002692 | >vvi-MIR166d | MI0006510 | >dsi-mir-311 | MI0009422 |
| >sla-mir-95 | MI0002693 | >vvi-MIR166e | MI0006511 | >dsi-mir-2b-1 | MI0009423 |
| >lla-mir-95 | MI0002694 | >vvi-MIR166f | MI0006512 | >dsi-mir-286 | MI0009424 |
| >ppa-mir-95 | MI0002695 | >vvi-MIR166g | MI0006513 | >dsi-mir-305 | MI0009425 |
| >mml-mir-98 | MI0002696 | >vvi-MIR166h | MI0006514 | >dsi-bantam | MI0009426 |
| >ptr-mir-98 | MI0002697 | >vvi-MIR167a | MI0006515 | >dsi-mir-3 | MI0009427 |
| >ggo-mir-98 | MI0002698 | >vvi-MIR167b | MI0006516 | >dsi-mir-317 | MI0009428 |
| >ppy-mir-98 | MI0002699 | >vvi-MIR167c | MI0006517 | >dsi-mir-314 | MI0009429 |
| >age-mir-98 | MI0002700 | >vvi-MIR167d | MI0006518 | >dsi-mir-11 | MI0009430 |
| >ppa-mir-98 | MI0002701 | >vvi-MIR167e | MI0006519 | >dsi-mir-33 | MI0009431 |
| >mml-mir-99a | MI0002702 | >vvi-MIR168 | MI0006520 | >dsi-mir-1 | MI0009432 |
| >ptr-mir-99a | MI0002703 | >vvi-MIR169a | MI0006521 | >dsi-mir-219 | MI0009433 |
| >ggo-mir-99a | MI0002704 | >vvi-MIR169y | MI0006522 | >dsi-mir-316 | MI0009434 |
| >ppy-mir-99a | MI0002705 | >vvi-MIR169c | MI0006523 | >dsi-mir-315 | MI0009435 |
| >lla-mir-99a | MI0002706 | >vvi-MIR169d | MI0006524 | >dsi-mir-263b | MI0009436 |
| >mne-mir-99a | MI0002707 | >vvi-MIR169e | MI0006525 | >dsi-mir-100 | MI0009437 |
| >ppa-mir-99a | MI0002708 | >vvi-MIR169f | MI0006526 | >dsi-mir-289 | MI0009438 |
| >ggo-mir-100 | MI0002709 | >vvi-MIR169g | MI0006527 | >dsi-mir-313 | MI0009439 |
| >age-mir-100 | MI0002710 | >vvi-MIR169j | MI0006528 | >dsi-mir-312 | MI0009440 |
| >ppa-mir-100 | MI0002711 | >vvi-MIR169k | MI0006529 | >dsi-let-7 | MI0009441 |
| >ppy-mir-100 | MI0002712 | >vvi-MIR169m | MI0006530 | >dsi-mir-308 | MI0009442 |
| >ptr-mir-100 | MI0002713 | >vvi-MIR169p | MI0006531 | >dsi-mir-2a-2 | MI0009443 |
| >mml-mir-100 | MI0002714 | >vvi-MIR169r | MI0006532 | >dsi-mir-31b | MI0009444 |
| >sla-mir-100 | MI0002715 | >vvi-MIR169s | MI0006533 | >dsi-mir-92b | MI0009445 |
| >lla-mir-100 | MI0002716 | >vvi-MIR169t | MI0006534 | >dsi-mir-275 | MI0009446 |
| >ggo-mir-101 | MI0002717 | >vvi-MIR169u | MI0006535 | >dsi-mir-13a | MI0009447 |
| >sla-mir-101 | MI0002718 | >vvi-MIR171a | MI0006536 | >dsi-mir-304 | MI0009448 |
| >age-mir-101 | MI0002719 | >vvi-MIR171b | MI0006537 | >dsi-mir-282 | MI0009449 |
| >ppa-mir-101 | MI0002720 | >vvi-MIR171c | MI0006538 | >dsi-mir-307 | MI0009450 |
| >ppy-mir-101 | MI0002721 | >vvi-MIR171d | MI0006539 | >dsi-mir-2b-2 | MI0009451 |
| >ptr-mir-101-1 | MI0002722 | >vvi-MIR171e | MI0006540 | >dsi-mir-6-1 | MI0009452 |
| >mml-mir-101-1 | MI0002723 | >vvi-MIR171f | MI0006541 | >dsi-mir-31a | MI0009453 |
| >lla-mir-101 | MI0002724 | >vvi-MIR171h | MI0006542 | >dsi-mir-10 | MI0009454 |
| >mne-mir-101 | MI0002725 | >vvi-MIR171i | MI0006543 | >dsi-mir-285 | MI0009455 |
| >ppy-mir-29b-1 | MI0002726 | >vvi-MIR172a | MI0006544 | >dsi-mir-8 | MI0009456 |
| >ptr-mir-29b-1 | MI0002727 | >vvi-MIR172b | MI0006545 | >dsi-mir-5 | MI0009457 |
| >ggo-mir-29b-1 | MI0002728 | >vvi-MIR172c | MI0006546 | >dsi-mir-278 | MI0009458 |
| >lla-mir-29b | MI0002729 | >vvi-MIR172d | MI0006547 | >dsi-mir-276a | MI0009459 |
| >age-mir-29b | MI0002730 | >vvi-MIR319b | MI0006548 | >dsi-mir-283 | MI0009460 |
| >ppa-mir-29b-1 | MI0002731 | >vvi-MIR319c | MI0006549 | >dsi-mir-309 | MI0009461 |
| >ptr-mir-29b-2 | MI0002732 | >vvi-MIR319f | MI0006550 | >dsi-mir-276b | MI0009462 |
| >ggo-mir-29b-2 | MI0002733 | >vvi-MIR319g | MI0006551 | >dsi-mir-279 | MI0009463 |
| >ppy-mir-29b-2 | MI0002734 | >vvi-MIR390 | MI0006552 | >dsi-mir-288 | MI0009464 |
| >sla-mir-29b | MI0002735 | >vvi-MIR393b | MI0006553 | >dsi-mir-92a | MI0009465 |
| >mne-mir-29b | MI0002736 | >vvi-MIR394a | MI0006554 | >dsi-mir-287 | MI0009466 |
| >ppa-mir-29b-2 | MI0002737 | >vvi-MIR394b | MI0006555 | >dsi-mir-6-3 | MI0009467 |
| >age-mir-103 | MI0002738 | >vvi-MIR395a | MI0006556 | >dsi-mir-133 | MI0009468 |
| >ggo-mir-103 | MI0002739 | >vvi-MIR395b | MI0006557 | >dsi-mir-7 | MI0009469 |
| >ppa-mir-103 | MI0002740 | >vvi-MIR395c | MI0006558 | >dsi-mir-284 | MI0009470 |
| >ppy-mir-103 | MI0002741 | >vvi-MIR395d | MI0006559 | >dsi-mir-184 | MI0009471 |
| >ptr-mir-103-1 | MI0002742 | >vvi-MIR395e | MI0006560 | >dsi-mir-281 | MI0009472 |
| >mml-mir-103-1 | MI0002743 | >vvi-MIR395f | MI0006561 | >dsi-mir-6-2 | MI0009473 |
| >lla-mir-103 | MI0002744 | >vvi-MIR395g | MI0006562 | >dsi-mir-318 | MI0009474 |
| >mne-mir-103 | MI0002745 | >vvi-MIR395h | MI0006563 | >dvi-mir-314 | MI0009475 |
| >ppy-mir-105 | MI0002746 | >vvi-MIR395i | MI0006564 | >dvi-mir-2a-2 | MI0009476 |
| >ggo-mir-105 | MI0002747 | >vvi-MIR395j | MI0006565 | >dvi-mir-2a-1 | MI0009477 |
| >ppa-mir-105 | MI0002748 | >vvi-MIR395k | MI0006566 | >dvi-mir-2c | MI0009478 |
| >ptr-mir-105 | MI0002749 | >vvi-MIR395l | MI0006567 | >dvi-mir-12 | MI0009479 |
| >mml-mir-105-1 | MI0002750 | >vvi-MIR395m | MI0006568 | >dvi-mir-13a | MI0009480 |
| >sla-mir-105 | MI0002751 | >vvi-MIR396a | MI0006569 | >dvi-mir-306 | MI0009481 |
| >lla-mir-105 | MI0002752 | >vvi-MIR396b | MI0006570 | >dvi-mir-279 | MI0009482 |
| >mne-mir-105 | MI0002753 | >vvi-MIR396d | MI0006571 | >dvi-mir-11 | MI0009483 |
| >mml-mir-107 | MI0002754 | >vvi-MIR398a | MI0006572 | >dvi-mir-125 | MI0009484 |
| >ptr-mir-107 | MI0002755 | >vvi-MIR399a | MI0006573 | >dvi-mir-313 | MI0009485 |
| >ggo-mir-107 | MI0002756 | >vvi-MIR399b | MI0006574 | >dvi-mir-263a | MI0009486 |
| >ppy-mir-107 | MI0002757 | >vvi-MIR399e | MI0006575 | >dvi-mir-274 | MI0009487 |
| >lla-mir-107 | MI0002758 | >vvi-MIR399g | MI0006576 | >dvi-mir-263b | MI0009488 |
| >mne-mir-107 | MI0002759 | >vvi-MIR399h | MI0006577 | >dvi-mir-318 | MI0009489 |
| >ppa-mir-107 | MI0002760 | >vvi-MIR408 | MI0006578 | >dvi-mir-6-2 | MI0009490 |
| >ggo-mir-124a | MI0002761 | >vvi-MIR479 | MI0006579 | >dvi-mir-9c | MI0009491 |
| >age-mir-124a | MI0002762 | >vvi-MIR535a | MI0006580 | >dvi-mir-9b | MI0009492 |
| >ppa-mir-124a | MI0002763 | >vvi-MIR535b | MI0006581 | >dvi-mir-281-2 | MI0009493 |
| >ppy-mir-124a | MI0002764 | >vvi-MIR535c | MI0006582 | >dvi-mir-210 | MI0009494 |
| >ptr-mir-124a | MI0002765 | >vvi-MIR535d | MI0006583 | >dvi-mir-9a | MI0009495 |
| >mml-mir-124a-1 | MI0002766 | >vvi-MIR535e | MI0006584 | >dvi-mir-287 | MI0009496 |
| >lla-mir-124a | MI0002767 | >age-mir-506 | MI0006585 | >dvi-mir-7 | MI0009497 |
| >lla-mir-139 | MI0002768 | >age-mir-507 | MI0006586 | >dvi-mir-282 | MI0009498 |
| >ppa-mir-139 | MI0002769 | >age-mir-508 | MI0006587 | >dvi-mir-315b | MI0009499 |
| >ptr-mir-147a | MI0002770 | >age-mir-509a | MI0006588 | >dvi-mir-310 | MI0009500 |
| >ppy-mir-147 | MI0002771 | >age-mir-509b | MI0006589 | >dvi-mir-283 | MI0009501 |
| >sla-mir-147 | MI0002772 | >age-mir-510 | MI0006590 | >dvi-mir-133 | MI0009502 |
| >mne-mir-147 | MI0002773 | >age-mir-513b | MI0006591 | >dvi-mir-278 | MI0009503 |
| >ppa-mir-147 | MI0002774 | >age-mir-513c-1 | MI0006592 | >dvi-mir-13b-1 | MI0009504 |
| >ggo-mir-7-1 | MI0002775 | >age-mir-513a-1 | MI0006593 | >dvi-mir-124 | MI0009505 |
| >ppy-mir-7-1 | MI0002776 | >age-mir-513d | MI0006594 | >dvi-mir-305 | MI0009506 |
| >sla-mir-7 | MI0002777 | >age-mir-513e-2 | MI0006595 | >dvi-mir-33 | MI0009507 |
| >lla-mir-7 | MI0002778 | >age-mir-513a-2 | MI0006596 | >dvi-mir-276b | MI0009508 |
| >mne-mir-7-1 | MI0002779 | >age-mir-513c-2 | MI0006597 | >dvi-mir-276a | MI0009509 |
| >ppa-mir-7-1 | MI0002780 | >age-mir-514 | MI0006598 | >dvi-mir-315a | MI0009510 |
| >ggo-mir-7-2 | MI0002781 | >ssy-mir-506 | MI0006599 | >dvi-mir-6-3 | MI0009511 |
| >ppy-mir-7-2 | MI0002782 | >ssy-mir-507 | MI0006600 | >dvi-mir-316 | MI0009512 |
| >mne-mir-7-2 | MI0002783 | >ssy-mir-508 | MI0006601 | >dvi-mir-277 | MI0009513 |
| >ppa-mir-7-2 | MI0002784 | >ssy-mir-509a | MI0006602 | >dvi-mir-281-1 | MI0009514 |
| >ptr-mir-7-3 | MI0002785 | >ssy-mir-509b | MI0006603 | >dvi-mir-317 | MI0009515 |
| >ggo-mir-7-3 | MI0002786 | >ssy-mir-510 | MI0006604 | >dvi-mir-184 | MI0009516 |
| >ppa-mir-7-3 | MI0002787 | >ssy-mir-513c | MI0006605 | >dvi-mir-286 | MI0009517 |
| >ggo-mir-10a | MI0002788 | >ssy-mir-513b-1 | MI0006606 | >dvi-mir-2b | MI0009518 |
| >ppy-mir-10a | MI0002789 | >ssy-mir-513a | MI0006607 | >dvi-mir-288 | MI0009519 |
| >sla-mir-10a | MI0002790 | >ssy-mir-513b-2 | MI0006608 | >dvi-mir-307 | MI0009520 |
| >age-mir-10a | MI0002791 | >ssy-mir-514 | MI0006609 | >dvi-mir-13b-2 | MI0009521 |
| >ppa-mir-10a | MI0002792 | >mml-mir-506 | MI0006610 | >dvi-mir-8 | MI0009522 |
| >ggo-mir-10b | MI0002793 | >mml-mir-507 | MI0006611 | >dvi-mir-34 | MI0009523 |
| >mne-mir-10b | MI0002794 | >mml-mir-508 | MI0006612 | >dvi-mir-4 | MI0009524 |
| >ppa-mir-10b | MI0002795 | >mml-mir-509-1 | MI0006613 | >dvi-mir-3 | MI0009525 |
| >ggo-mir-34a | MI0002796 | >mml-mir-509-2 | MI0006614 | >dvi-mir-309 | MI0009526 |
| >age-mir-34a | MI0002797 | >mml-mir-510 | MI0006615 | >dvi-mir-1 | MI0009527 |
| >ppa-mir-34a | MI0002798 | >mml-mir-513b-1 | MI0006616 | >dvi-mir-10 | MI0009528 |
| >ppy-mir-34a | MI0002799 | >mml-mir-513b-2 | MI0006617 | >dvi-mir-5 | MI0009529 |
| >ptr-mir-34a | MI0002800 | >mml-mir-513a-1 | MI0006618 | >dvi-mir-92b | MI0009530 |
| >mml-mir-34a | MI0002801 | >mml-mir-513a-2 | MI0006619 | >dvi-mir-iab-4 | MI0009531 |
| >sla-mir-34a | MI0002802 | >mml-mir-513a-3 | MI0006620 | >dvi-mir-280 | MI0009532 |
| >lla-mir-34a | MI0002803 | >mml-mir-514-1 | MI0006621 | >dvi-bantam | MI0009533 |
| >mne-mir-34a | MI0002804 | >ptr-mir-506 | MI0006622 | >dvi-mir-31a | MI0009534 |
| >ggo-mir-181a-2 | MI0002805 | >ptr-mir-507 | MI0006623 | >dvi-mir-308 | MI0009535 |
| >ppa-mir-181a-2 | MI0002806 | >ptr-mir-508 | MI0006624 | >dvi-mir-285 | MI0009536 |
| >ptr-mir-181a-2 | MI0002807 | >ptr-mir-509a-1 | MI0006625 | >dvi-mir-6-1 | MI0009537 |
| >mml-mir-181a-2 | MI0002808 | >ptr-mir-509b | MI0006626 | >dvi-mir-275 | MI0009538 |
| >sla-mir-181a-2 | MI0002809 | >ptr-mir-509a-2 | MI0006627 | >dvi-mir-14 | MI0009539 |
| >mne-mir-181a-2 | MI0002810 | >ptr-mir-510 | MI0006628 | >dvi-mir-31b | MI0009540 |
| >mml-mir-181c | MI0002811 | >ptr-mir-513a-1 | MI0006629 | >dvi-mir-100 | MI0009541 |
| >ptr-mir-181c | MI0002812 | >ptr-mir-513b | MI0006630 | >dvi-mir-87 | MI0009542 |
| >ggo-mir-181c | MI0002813 | >ptr-mir-513a-2 | MI0006631 | >dvi-mir-79 | MI0009543 |
| >ppa-mir-181c | MI0002814 | >ptr-mir-513a-3 | MI0006632 | >dvi-mir-304 | MI0009544 |
| >mml-mir-182 | MI0002815 | >ptr-mir-514-1 | MI0006633 | >dvi-mir-284 | MI0009545 |
| >ppy-mir-182 | MI0002816 | >ptr-mir-514-2 | MI0006634 | >dvi-mir-219 | MI0009546 |
| >ggo-mir-187 | MI0002817 | >ptr-mir-514-3 | MI0006635 | >dvi-mir-92a | MI0009547 |
| >ppy-mir-187 | MI0002818 | >ptr-mir-514-4 | MI0006636 | >dvi-let-7 | MI0009548 |
| >mne-mir-187 | MI0002819 | >pbi-mir-506 | MI0006637 | >dwi-mir-79-2 | MI0009549 |
| >ppa-mir-187 | MI0002820 | >pbi-mir-507 | MI0006638 | >dwi-mir-87-2 | MI0009550 |
| >mml-mir-196a-1 | MI0002821 | >pbi-mir-508 | MI0006639 | >dwi-mir-34 | MI0009551 |
| >ggo-mir-196-1 | MI0002822 | >pbi-mir-509 | MI0006640 | >dwi-mir-285 | MI0009552 |
| >ppy-mir-196-1 | MI0002823 | >pbi-mir-510 | MI0006641 | >dwi-bantam | MI0009553 |
| >ptr-mir-196a-2 | MI0002824 | >pbi-mir-513a-1 | MI0006642 | >dwi-mir-306-1 | MI0009554 |
| >ggo-mir-196-2 | MI0002825 | >pbi-mir-513b | MI0006643 | >dwi-mir-1 | MI0009555 |
| >ppy-mir-196-2 | MI0002826 | >pbi-mir-513c-1 | MI0006644 | >dwi-mir-184 | MI0009556 |
| >lla-mir-196 | MI0002827 | >pbi-mir-513a-2 | MI0006645 | >dwi-mir-13b-1 | MI0009557 |
| >age-mir-196 | MI0002828 | >pbi-mir-513c-2 | MI0006646 | >dwi-mir-92b | MI0009558 |
| >ppa-mir-196 | MI0002829 | >pbi-mir-514 | MI0006647 | >dwi-mir-9c-1 | MI0009559 |
| >ggo-mir-199a | MI0002830 | >hsa-mir-513b | MI0006648 | >dwi-mir-284 | MI0009560 |
| >ppa-mir-199a | MI0002831 | >hsa-mir-513c | MI0006649 | >dwi-mir-281-2 | MI0009561 |
| >ppy-mir-199a | MI0002832 | >age-mir-513e-1 | MI0006650 | >dwi-let-7 | MI0009562 |
| >ptr-mir-199a-2 | MI0002833 | >hsa-mir-1321 | MI0006652 | >dwi-mir-305 | MI0009563 |
| >mml-mir-199a-1 | MI0002834 | >hsa-mir-1322 | MI0006653 | >dwi-mir-92a | MI0009564 |
| >sla-mir-199a | MI0002835 | >hsa-mir-720 | MI0006654 | >dwi-mir-286 | MI0009565 |
| >lla-mir-199a | MI0002836 | >hsa-mir-1197 | MI0006656 | >dwi-mir-316 | MI0009566 |
| >mne-mir-199a | MI0002837 | >hsa-mir-1324 | MI0006657 | >dwi-mir-279 | MI0009567 |
| >ptr-mir-204 | MI0002838 | >oan-mir-9 | MI0006658 | >dwi-mir-263b | MI0009568 |
| >ggo-mir-204 | MI0002839 | >oan-mir-1325 | MI0006659 | >dwi-mir-287 | MI0009569 |
| >ppy-mir-204 | MI0002840 | >oan-mir-1326 | MI0006660 | >dwi-mir-iab-4 | MI0009570 |
| >sla-mir-204 | MI0002841 | >oan-mir-1327 | MI0006661 | >dwi-mir-2a-2 | MI0009571 |
| >mne-mir-204 | MI0002842 | >oan-mir-1328 | MI0006662 | >dwi-mir-276a | MI0009572 |
| >ppa-mir-204 | MI0002843 | >oan-mir-1329 | MI0006663 | >dwi-mir-7 | MI0009573 |
| >ggo-mir-205 | MI0002844 | >oan-mir-1330 | MI0006664 | >dwi-mir-306-2 | MI0009574 |
| >age-mir-205 | MI0002845 | >oan-mir-129 | MI0006665 | >dwi-mir-2a-1 | MI0009575 |
| >ppa-mir-205 | MI0002846 | >oan-mir-96 | MI0006666 | >dwi-mir-11 | MI0009576 |
| >ptr-mir-205 | MI0002847 | >oan-mir-183 | MI0006667 | >dwi-mir-304 | MI0009577 |
| >lla-mir-205 | MI0002848 | >oan-mir-29a-1 | MI0006668 | >dwi-mir-219 | MI0009578 |
| >mne-mir-205 | MI0002849 | >oan-mir-29b | MI0006669 | >dwi-mir-79-1 | MI0009579 |
| >mml-mir-211 | MI0002850 | >oan-mir-490 | MI0006670 | >dwi-mir-8 | MI0009580 |
| >ppy-mir-211 | MI0002851 | >oan-mir-140 | MI0006671 | >dwi-mir-283 | MI0009581 |
| >mne-mir-211 | MI0002852 | >oan-mir-1331 | MI0006672 | >dwi-mir-318-2 | MI0009582 |
| >ggo-mir-214 | MI0002853 | >oan-mir-218 | MI0006673 | >dwi-mir-2b | MI0009583 |
| >age-mir-214 | MI0002854 | >oan-mir-33a | MI0006674 | >dwi-mir-12 | MI0009584 |
| >ppa-mir-214 | MI0002855 | >oan-mir-1332 | MI0006675 | >dwi-mir-276b | MI0009585 |
| >ppy-mir-214 | MI0002856 | >oan-mir-193 | MI0006676 | >dwi-mir-210 | MI0009586 |
| >ptr-mir-214 | MI0002857 | >oan-mir-365 | MI0006677 | >dwi-mir-309 | MI0009587 |
| >mml-mir-214 | MI0002858 | >oan-mir-1333 | MI0006678 | >dwi-mir-6-3 | MI0009588 |
| >sla-mir-214 | MI0002859 | >oan-mir-122 | MI0006679 | >dwi-mir-274 | MI0009589 |
| >mne-mir-214 | MI0002860 | >oan-mir-1334 | MI0006680 | >dwi-mir-317 | MI0009590 |
| >lca-mir-216 | MI0002861 | >oan-mir-1335 | MI0006681 | >dwi-mir-9c-2 | MI0009591 |
| >ptr-mir-216a | MI0002862 | >oan-mir-1336 | MI0006682 | >dwi-mir-314 | MI0009592 |
| >ggo-mir-216 | MI0002863 | >oan-mir-875 | MI0006683 | >dwi-mir-282 | MI0009593 |
| >ppy-mir-216 | MI0002864 | >oan-mir-1337 | MI0006684 | >dwi-mir-124 | MI0009594 |
| >ppa-mir-216 | MI0002865 | >oan-mir-181a | MI0006685 | >dwi-mir-280 | MI0009595 |
| >ggo-mir-217 | MI0002866 | >oan-mir-181b | MI0006686 | >dwi-mir-278 | MI0009596 |
| >ppa-mir-217 | MI0002867 | >oan-mir-137a | MI0006687 | >dwi-mir-13a | MI0009597 |
| >ggo-mir-218 | MI0002868 | >oan-mir-1338 | MI0006688 | >dwi-mir-307 | MI0009598 |
| >age-mir-218 | MI0002869 | >oan-mir-204 | MI0006689 | >dwi-mir-125 | MI0009599 |
| >ppa-mir-218-1 | MI0002870 | >oan-mir-190a | MI0006690 | >dwi-mir-10 | MI0009600 |
| >lca-mir-218 | MI0002871 | >oan-mir-15c | MI0006691 | >dwi-mir-9a | MI0009601 |
| >ppy-mir-218-1 | MI0002872 | >oan-mir-16c | MI0006692 | >dwi-mir-318-1 | MI0009602 |
| >ptr-mir-218-1 | MI0002873 | >oan-mir-106 | MI0006693 | >dwi-mir-2c | MI0009603 |
| >sla-mir-218-1 | MI0002874 | >oan-mir-18 | MI0006694 | >dwi-mir-315 | MI0009604 |
| >lla-mir-218-1 | MI0002875 | >oan-mir-20b | MI0006695 | >dwi-mir-31b | MI0009605 |
| >mne-mir-218-1 | MI0002876 | >oan-mir-19b | MI0006696 | >dwi-mir-13b-2 | MI0009606 |
| >ppa-mir-218-2 | MI0002877 | >oan-mir-92a-1 | MI0006697 | >dwi-mir-308 | MI0009607 |
| >ppy-mir-218-2 | MI0002878 | >oan-mir-363 | MI0006698 | >dwi-mir-87-1 | MI0009608 |
| >ptr-mir-218-2 | MI0002879 | >oan-mir-1339 | MI0006699 | >dwi-mir-6-2 | MI0009609 |
| >mml-mir-218-2 | MI0002880 | >oan-mir-1a | MI0006700 | >dwi-mir-263a | MI0009610 |
| >sla-mir-218-2 | MI0002881 | >oan-mir-133a | MI0006701 | >dwi-mir-6-1 | MI0009611 |
| >lla-mir-218-2 | MI0002882 | >oan-mir-29a-2 | MI0006702 | >dwi-mir-277 | MI0009612 |
| >mne-mir-218-2 | MI0002883 | >oan-mir-205 | MI0006703 | >dwi-mir-5 | MI0009613 |
| >mml-mir-219-1 | MI0002884 | >oan-mir-1340 | MI0006704 | >dwi-mir-33 | MI0009614 |
| >ggo-mir-219 | MI0002885 | >oan-mir-135b | MI0006705 | >dwi-mir-14 | MI0009615 |
| >ppy-mir-219 | MI0002886 | >oan-mir-1341 | MI0006706 | >dwi-mir-31a | MI0009616 |
| >mml-mir-220a | MI0002887 | >oan-mir-1342 | MI0006707 | >dwi-mir-275 | MI0009617 |
| >ptr-mir-220a | MI0002888 | >oan-mir-1343 | MI0006708 | >dwi-mir-3 | MI0009618 |
| >ggo-mir-220 | MI0002889 | >oan-mir-196a | MI0006709 | >dwi-mir-9b-1 | MI0009619 |
| >mne-mir-220 | MI0002890 | >oan-mir-137b | MI0006710 | >dwi-mir-281-1 | MI0009620 |
| >ppa-mir-220 | MI0002891 | >oan-mir-1422a | MI0006711 | >dwi-mir-9b-2 | MI0009621 |
| >mml-mir-221 | MI0002892 | >oan-mir-1422e | MI0006712 | >dwi-mir-100 | MI0009622 |
| >ggo-mir-221 | MI0002893 | >oan-mir-1422h | MI0006713 | >dwi-mir-4 | MI0009623 |
| >ppy-mir-221 | MI0002894 | >oan-mir-1422b-1 | MI0006714 | >dwi-mir-288 | MI0009624 |
| >ppa-mir-221 | MI0002895 | >oan-mir-1422f | MI0006715 | >dwi-mir-133 | MI0009625 |
| >age-mir-222 | MI0002896 | >oan-mir-1422g | MI0006716 | >dya-mir-274 | MI0009626 |
| >mml-mir-223 | MI0002897 | >oan-mir-1422c | MI0006717 | >dya-mir-87 | MI0009627 |
| >ptr-mir-223 | MI0002898 | >oan-mir-1422i | MI0006718 | >dya-mir-307 | MI0009628 |
| >ggo-mir-223 | MI0002899 | >oan-mir-1422k | MI0006719 | >dya-mir-317 | MI0009629 |
| >ppy-mir-223 | MI0002900 | >oan-let-7b | MI0006720 | >dya-mir-13b-2 | MI0009630 |
| >sla-mir-223 | MI0002901 | >oan-mir-219 | MI0006721 | >dya-bantam | MI0009631 |
| >ppa-mir-223 | MI0002902 | >oan-mir-451 | MI0006722 | >dya-mir-4 | MI0009632 |
| >mml-mir-224 | MI0002903 | >oan-mir-144 | MI0006723 | >dya-mir-100 | MI0009633 |
| >ptr-mir-224 | MI0002904 | >oan-mir-139 | MI0006724 | >dya-mir-281-2 | MI0009634 |
| >ggo-mir-224 | MI0002905 | >oan-mir-1344 | MI0006725 | >dya-mir-279 | MI0009635 |
| >ppy-mir-224 | MI0002906 | >oan-mir-130c | MI0006726 | >dya-mir-33 | MI0009636 |
| >mne-mir-224 | MI0002907 | >oan-mir-301 | MI0006727 | >dya-mir-283 | MI0009637 |
| >ppa-mir-224 | MI0002908 | >oan-mir-30c | MI0006728 | >dya-mir-280 | MI0009638 |
| >ptr-mir-197 | MI0002909 | >oan-mir-30a | MI0006729 | >dya-mir-8 | MI0009639 |
| >ppy-mir-197 | MI0002910 | >oan-mir-460 | MI0006730 | >dya-mir-305 | MI0009640 |
| >mne-mir-197 | MI0002911 | >oan-mir-1345 | MI0006731 | >dya-mir-306 | MI0009641 |
| >age-mir-197 | MI0002912 | >oan-mir-92b | MI0006732 | >dya-mir-9a-1 | MI0009642 |
| >ppa-mir-197 | MI0002913 | >oan-mir-1346 | MI0006733 | >dya-mir-282 | MI0009643 |
| >ggo-mir-198 | MI0002914 | >oan-mir-208 | MI0006734 | >dya-mir-133 | MI0009644 |
| >age-mir-198 | MI0002915 | >oan-mir-1420a | MI0006735 | >dya-mir-304 | MI0009645 |
| >ppa-mir-198 | MI0002916 | >oan-mir-1420b | MI0006736 | >dya-mir-3 | MI0009646 |
| >ppy-mir-198 | MI0002917 | >oan-mir-1420c | MI0006737 | >dya-mir-2b-2 | MI0009647 |
| >ptr-mir-198 | MI0002918 | >oan-mir-1420d | MI0006738 | >dya-mir-14 | MI0009648 |
| >mml-mir-198 | MI0002919 | >oan-mir-1420e | MI0006739 | >dya-mir-314 | MI0009649 |
| >sla-mir-198 | MI0002920 | >oan-mir-1347 | MI0006740 | >dya-mir-6-3 | MI0009650 |
| >lla-mir-198 | MI0002921 | >oan-mir-186 | MI0006741 | >dya-mir-318 | MI0009651 |
| >mne-mir-198 | MI0002922 | >oan-mir-1348 | MI0006742 | >dya-mir-316-1 | MI0009652 |
| >ptr-mir-30c-2 | MI0002923 | >oan-mir-1349 | MI0006743 | >dya-mir-34 | MI0009653 |
| >lla-mir-30c | MI0002924 | >oan-mir-200a | MI0006744 | >dya-mir-10 | MI0009654 |
| >mne-mir-30c | MI0002925 | >oan-mir-200b | MI0006745 | >dya-mir-1 | MI0009655 |
| >ptr-mir-30d | MI0002926 | >oan-mir-14221 | MI0006746 | >dya-mir-315-2 | MI0009656 |
| >ggo-mir-30d | MI0002927 | >oan-mir-1350 | MI0006747 | >dya-mir-309 | MI0009657 |
| >mne-mir-30d | MI0002928 | >oan-mir-103-2 | MI0006748 | >dya-mir-13b-1 | MI0009658 |
| >ppa-mir-30d | MI0002929 | >oan-mir-1351 | MI0006749 | >dya-mir-278 | MI0009659 |
| >ppa-mir-1 | MI0002930 | >oan-mir-1420f | MI0006750 | >dya-mir-284 | MI0009660 |
| >mml-mir-181a-1 | MI0002931 | >oan-mir-1352 | MI0006751 | >dya-mir-285 | MI0009661 |
| >mml-mir-181b-1 | MI0002932 | >oan-mir-1353 | MI0006752 | >dya-mir-2b-1 | MI0009662 |
| >ptr-mir-181a-1 | MI0002933 | >oan-mir-1354 | MI0006753 | >dya-mir-iab-4 | MI0009663 |
| >ptr-mir-181b-1 | MI0002934 | >oan-mir-499 | MI0006754 | >dya-mir-311a | MI0009664 |
| >ppy-mir-181a-1 | MI0002935 | >oan-mir-1355 | MI0006755 | >dya-mir-310 | MI0009665 |
| >ppy-mir-181b | MI0002936 | >oan-mir-1356 | MI0006756 | >dya-mir-281-1 | MI0009666 |
| >ggo-mir-181a-1 | MI0002937 | >oan-mir-24 | MI0006757 | >dya-mir-13a | MI0009667 |
| >ggo-mir-181b | MI0002938 | >oan-mir-27b | MI0006758 | >dya-mir-288 | MI0009668 |
| >lla-mir-181a-1 | MI0002939 | >oan-mir-23b | MI0006759 | >dya-mir-210 | MI0009669 |
| >lla-mir-181b | MI0002940 | >oan-mir-1357 | MI0006760 | >dya-mir-125 | MI0009670 |
| >mne-mir-181a-1 | MI0002941 | >oan-mir-22 | MI0006761 | >dya-mir-6-1 | MI0009671 |
| >mne-mir-181b | MI0002942 | >oan-mir-1421a | MI0006762 | >dya-mir-2c | MI0009672 |
| >ppa-mir-181a-1 | MI0002943 | >oan-mir-1421b | MI0006763 | >dya-let-7 | MI0009673 |
| >ppa-mir-181b | MI0002944 | >oan-mir-1421c | MI0006764 | >dya-mir-277 | MI0009674 |
| >age-mir-15a | MI0002945 | >oan-mir-1421d | MI0006765 | >dya-mir-308 | MI0009675 |
| >age-mir-16 | MI0002946 | >oan-mir-1421e | MI0006766 | >dya-mir-5 | MI0009676 |
| >ggo-mir-15a | MI0002947 | >oan-mir-1421f | MI0006767 | >dya-mir-287 | MI0009677 |
| >ggo-mir-16 | MI0002948 | >oan-mir-1421g-1 | MI0006768 | >dya-mir-31a | MI0009678 |
| >mne-mir-15a | MI0002949 | >oan-mir-1421h | MI0006769 | >dya-mir-31b | MI0009679 |
| >mne-mir-16 | MI0002950 | >oan-mir-1421i-1 | MI0006770 | >dya-mir-124 | MI0009680 |
| >sla-mir-15a | MI0002951 | >oan-mir-1421j | MI0006771 | >dya-mir-2a-2 | MI0009681 |
| >sla-mir-16 | MI0002952 | >oan-mir-142 | MI0006772 | >dya-mir-219 | MI0009682 |
| >ppa-mir-15a | MI0002953 | >oan-let-7e | MI0006773 | >dya-mir-312 | MI0009683 |
| >ppa-mir-16 | MI0002954 | >oan-mir-7-2 | MI0006774 | >dya-mir-286 | MI0009684 |
| >lca-mir-15a | MI0002955 | >oan-mir-155 | MI0006775 | >dya-mir-315-1 | MI0009685 |
| >lca-mir-16 | MI0002956 | >oan-mir-1358 | MI0006776 | >dya-mir-9a-2 | MI0009686 |
| >mml-mir-15a | MI0002957 | >oan-mir-26 | MI0006777 | >dya-mir-263b-1 | MI0009687 |
| >mml-mir-16-1 | MI0002958 | >oan-mir-1359 | MI0006778 | >dya-mir-92a | MI0009688 |
| >ppy-mir-15a | MI0002959 | >oan-mir-206 | MI0006779 | >dya-mir-9c | MI0009689 |
| >ppy-mir-16 | MI0002960 | >oan-mir-133b | MI0006780 | >dya-mir-2a-1 | MI0009690 |
| >ptr-mir-15a | MI0002961 | >oan-mir-190b | MI0006781 | >dya-mir-9b | MI0009691 |
| >ptr-mir-16-1 | MI0002962 | >oan-mir-194-1 | MI0006782 | >dya-mir-263a | MI0009692 |
| >lla-mir-15a | MI0002963 | >oan-mir-215 | MI0006783 | >dya-mir-275 | MI0009693 |
| >lla-mir-16 | MI0002964 | >oan-mir-1360 | MI0006784 | >dya-mir-6-2 | MI0009694 |
| >ggo-mir-17 | MI0002965 | >oan-mir-147 | MI0006785 | >dya-mir-316-2 | MI0009695 |
| >ggo-mir-18 | MI0002966 | >oan-mir-153 | MI0006786 | >dya-mir-311b | MI0009696 |
| >ggo-mir-19a | MI0002967 | >oan-mir-128-1 | MI0006787 | >dya-mir-276a | MI0009697 |
| >ggo-mir-20 | MI0002968 | >oan-mir-146b | MI0006788 | >dya-mir-289 | MI0009698 |
| >ggo-mir-19b-1 | MI0002969 | >oan-mir-458 | MI0006789 | >dya-mir-263b-2 | MI0009699 |
| >ggo-mir-92-1 | MI0002970 | >oan-mir-101-1 | MI0006790 | >dya-mir-7 | MI0009700 |
| >lca-mir-17 | MI0002971 | >oan-mir-449c | MI0006791 | >dya-mir-92b | MI0009701 |
| >lca-mir-18 | MI0002972 | >oan-mir-449b | MI0006792 | >dya-mir-184 | MI0009702 |
| >lca-mir-19a | MI0002973 | >oan-mir-449a | MI0006793 | >dya-mir-11 | MI0009703 |
| >lca-mir-20 | MI0002974 | >oan-mir-192 | MI0006794 | >dya-mir-79 | MI0009704 |
| >lca-mir-19b-1 | MI0002975 | >oan-mir-194-2 | MI0006795 | >dya-mir-276b | MI0009705 |
| >lca-mir-92-1 | MI0002976 | >oan-mir-223 | MI0006796 | >pta-MIR1309 | MI0009706 |
| >age-mir-17 | MI0002977 | >oan-mir-1361 | MI0006797 | >pta-MIR1310 | MI0009707 |
| >age-mir-18 | MI0002978 | >oan-mir-1362 | MI0006798 | >pta-MIR1311 | MI0009708 |
| >age-mir-19a | MI0002979 | >oan-mir-1363 | MI0006799 | >pta-MIR1312 | MI0009709 |
| >age-mir-20 | MI0002980 | >oan-mir-1364 | MI0006800 | >pta-MIR1313 | MI0009710 |
| >age-mir-19b-1 | MI0002981 | >oan-mir-1365 | MI0006801 | >pta-MIR1314 | MI0009711 |
| >age-mir-92-1 | MI0002982 | >oan-mir-1366 | MI0006802 | >pta-MIR1315 | MI0009712 |
| >ppa-mir-17 | MI0002983 | >oan-mir-1367 | MI0006803 | >pta-MIR1316 | MI0009713 |
| >ppa-mir-18 | MI0002984 | >oan-mir-1368 | MI0006804 | >osa-MIR1317 | MI0009714 |
| >ppa-mir-19a | MI0002985 | >oan-mir-1369 | MI0006805 | >osa-MIR1318 | MI0009715 |
| >ppa-mir-20 | MI0002986 | >oan-mir-1370 | MI0006806 | >osa-MIR1319 | MI0009716 |
| >ppa-mir-19b-1 | MI0002987 | >oan-mir-1371 | MI0006807 | >osa-MIR1320 | MI0009717 |
| >ppa-mir-92-1 | MI0002988 | >oan-mir-30f | MI0006808 | >sly-MIR156a | MI0009971 |
| >ppy-mir-17 | MI0002989 | >oan-let-7g | MI0006809 | >sly-MIR156b | MI0009972 |
| >ppy-mir-18 | MI0002990 | >oan-mir-1372 | MI0006810 | >sly-MIR156c | MI0009973 |
| >ppy-mir-19a | MI0002991 | >oan-mir-33b | MI0006811 | >sly-MIR159 | MI0009974 |
| >ppy-mir-20 | MI0002992 | >oan-mir-30d | MI0006812 | >sly-MIR162 | MI0009975 |
| >ppy-mir-19b-1 | MI0002993 | >oan-mir-30b | MI0006813 | >sly-MIR172a | MI0009976 |
| >ppy-mir-92-1 | MI0002994 | >oan-mir-184 | MI0006814 | >sly-MIR172b | MI0009977 |
| >ptr-mir-17 | MI0002995 | >oan-mir-100 | MI0006815 | >sly-MIR319 | MI0009978 |
| >ptr-mir-18a | MI0002996 | >oan-mir-125 | MI0006816 | >sly-MIR399 | MI0009979 |
| >ptr-mir-19a | MI0002997 | >oan-mir-15b | MI0006818 | >jcv-mir-J1 | MI0009980 |
| >ptr-mir-20a | MI0002998 | >oan-mir-16b | MI0006819 | >bkv-mir-B1 | MI0009981 |
| >ptr-mir-19b-1 | MI0002999 | >oan-mir-1373 | MI0006820 | >bmo-mir-12 | MI0009994 |
| >ptr-mir-92-1 | MI0003000 | >oan-mir-1374 | MI0006821 | >bmo-mir-137 | MI0009995 |
| >mml-mir-17 | MI0003001 | >oan-mir-222b | MI0006822 | >bmo-mir-190 | MI0009996 |
| >mml-mir-18 | MI0003002 | >oan-mir-181c | MI0006823 | >bmo-mir-285 | MI0009997 |
| >mml-mir-19a | MI0003003 | >oan-mir-23a | MI0006824 | >bmo-mir-92 | MI0009998 |
| >mml-mir-20a | MI0003004 | >oan-mir-27a | MI0006825 | >bmo-mir-925 | MI0009999 |
| >mml-mir-19b-1 | MI0003005 | >oan-mir-1375 | MI0006826 | >bmo-mir-927 | MI0010000 |
| >mml-mir-92a-1 | MI0003006 | >oan-mir-1421k-1 | MI0006827 | >bmo-mir-929 | MI0010001 |
| >sla-mir-17 | MI0003007 | >oan-mir-1421l-1 | MI0006828 | >bmo-mir-930 | MI0010002 |
| >sla-mir-18 | MI0003008 | >oan-mir-1419a | MI0006829 | >bmo-mir-932 | MI0010003 |
| >sla-mir-19a | MI0003009 | >oan-mir-1421m | MI0006830 | >bmo-mir-9b | MI0010004 |
| >sla-mir-20 | MI0003010 | >oan-mir-1421n-1 | MI0006831 | >bmo-mir-iab-4 | MI0010005 |
| >sla-mir-19b-1 | MI0003011 | >oan-mir-1421o | MI0006832 | >bmo-mir-278 | MI0010006 |
| >sla-mir-92-1 | MI0003012 | >oan-mir-1421p-1 | MI0006833 | >osa-MIR1846c | MI0010321 |

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-86. (canceled)

87. A method of detecting the presence of a virus in a biological sample, comprising determining a miRNA profile for the sample and comparing the miRNA profile to a previously identified virus-specific miRNA signature, wherein a substantial similarity in said profiles indicates the presence of the virus in the sample.

88. A method of detecting the presence of a virus in a biological sample, comprising measuring in the sample the level of at least one miRNA associated with a virus-specific miRNA signature, wherein an alteration in the level of the miRNA in the sample relative to the level of corresponding miRNA in a control sample is indicative of the sample containing the virus.

89. A method of detecting the replication stage of a virus in a biological sample, comprising determining a miRNA profile for the sample and comparing the miRNA profile to a previously identified virus-specific, replication stage-specific miRNA signature, wherein a substantial similarity in said profiles indicates the replication stage of a virus in the sample.

90. A method of detecting the replication stage of a virus in a biological sample, comprising measuring in the sample the level of at least one miRNA associated with a virus-specific replication stage-specific miRNA signature, wherein an alteration in the level of the miRNA in the sample relative to the level of corresponding miRNA in a control sample is indicative of the replication stage of a virus in the sample.

91. The method of claim 87, wherein the virus-specific miRNA signature is identified using a method comprising:
(a) generating a miRNA profile from a test sample obtained from a cell infected with a virus; and
(b) comparing the test sample a miRNA profile to an appropriate control sample miRNA profile;
wherein one or more alterations between the test sample miRNA profile and the control sample miRNA profile defines the virus-specific miRNA signature.

92. The method of claim 89, wherein the virus-specific, replication stage-specific miRNA signature is identified using a method comprising:
(a) generating a miRNA profile from a test sample obtained from a cell infected with a virus, wherein said virus is in a replication stage selected from the group consisting of productive, persistent and latent; and
(b) comparing the test sample a miRNA profile to an appropriate control sample miRNA profile;
wherein one or more alterations between the test sample a miRNA profile and the control sample miRNA profile defines the virus-specific, replication stage-specific miRNA signature.

93. The method of claim 87 or 89, wherein the cell is selected from the group consisting of a eukaryotic cell, a mammalian cell, an avian cell, and a human cell.

94-96. (canceled)

97. The method of claim 93, wherein the cell is obtained from a source selected from the group consisting of a biopsy sample, a blood sample, a non-blood fluid sample, and a subject following organ transplantation.

98. (canceled)

99. The method of claim 97, wherein the organ is selected from the group consisting of kidney, liver, heart, lung and skin.

100. The method of claim 87 or 89, wherein the virus is capable of infecting eukaryotic cells or mammalian cells.

101. (canceled)

102. The method of claim 100, wherein the virus belongs to a family selected from the group consisting of the Herpesviridae, Retroviridae, Reoviridae, Adenoviridae, Flaviviridae, Poxyiridae, Caliciviridae, Togaviridae, Coronaviridae, Rhabdoviridae, Filoviridae, Paramyxoviridae, Orthomyxoviridae, Bunyaviridae, Arenaviridae, Bornaviridae, Polyomaviridae, Papillomaviridae, Parvoviridae, Hepadnaviridae and Picornaviridae families.

103. The method of claim 100, wherein the virus is selected from the group consisting of Adenovirus, Cytomegalovirus (e.g., HCMV), Epstein Barr virus (EBV), Human Papilloma virus (HPV), MHV-68, Human Immunodeficiency Virus (HIV), Hepatitis A Virus (HAV), Hepatitis B Virus (HBV), Hepatitis C Virus (HCV), Hepatitis E Virus (HEV), Rubella Virus, Mumps Virus, Measles Virus, Respiratory Syncytial Virus, Human T-cell Leukemia Virus, Lentivirus, Herpes Simplex Virus (e.g., Herpes Simplex 1 (HSV1), Herpes Simplex 2 (HSV2)), Varicella-Zoster Virus, Human Herpesviruses 6A, 6B, and 7, Kaposi's Sarcoma-Associated Herpesvirus (e.g., KSHV, HHV8), Cercopithecine Herpesvirus, Hepatitis Delta Virus, Dengue Virus, Foot and Mouth Disease Virus, Polyomavirus (e.g., JC, BK), Poliovirus, Coxsackievirus, Echovirus, Rhinovirus, Vacciniavirus, Small Pox Virus, Influenza Virus, and Avian Influenza Virus.

104. The method of claim 100, wherein the virus belongs to the Adenoviridae family, for example, wherein the virus is Adenovirus type 5.

105. (canceled)

106. The method of claim 100, wherein the virus belongs to the Herpesviridae family, for example, wherein the virus is Human cytomegalovirus.

107-113. (canceled)

114. A microarray comprising miRNA-specific probe oligonucleotides for the miRNAs of the miRNA signature identified according to a method selected from the group consisting of:
(a) a method of identifying a virus-specific miRNA signature, comprising generating a miRNA profile from a test sample obtained from a cell infected with a virus and comparing the test sample a miRNA profile to an appropriate control sample miRNA profile, wherein one or more alterations between the test sample miRNA profile and the control sample miRNA profile defines the virus-specific miRNA signature;
(b) a method of identifying a virus-specific, replication stage-specific miRNA signature, comprising generating a miRNA profile from a test sample obtained from a cell infected with a virus, wherein said virus is in a replication stage selected from the group consisting of productive, persistent and latent, and comparing the test sample a miRNA profile to an appropriate control sample miRNA profile, wherein one or more alterations between the test sample a miRNA profile and the control sample miRNA profile defines the virus-specific, replication stage-specific miRNA signature; and
(c) A method of identifying an miRNA signature associated with a specific disease or pathological state of a subject infected with a virus, comprising generating a miRNA profile from a test sample obtained from the subject, and comparing the test sample miRNA profile to an appropriate control sample miRNA profile, wherein one or more alterations between the test sample miRNA profile and the control sample miRNA profile defines the miRNA signature associated with the disease or pathological state.

115. A set of oligonucleotide primers designed to specifically amplify the miRNAs of the miRNA signature identified according to method selected from the group consisting of:
(a) a method of identifying a virus-specific miRNA signature, comprising generating a miRNA profile from a test sample obtained from a cell infected with a virus and comparing the test sample a miRNA profile to an appropriate control sample miRNA profile, wherein one or more alterations between the test sample miRNA profile and the control sample miRNA profile defines the virus-specific miRNA signature;
(b) a method of identifying a virus-specific, replication stage-specific miRNA signature, comprising generating a miRNA profile from a test sample obtained from a cell infected with a virus, wherein said virus is in a replication stage selected from the group consisting of productive, persistent and latent, and comparing the test sample a miRNA profile to an appropriate control sample miRNA profile, wherein one or more alterations between the test sample a miRNA profile and the control sample miRNA profile defines the virus-specific, replication stage-specific miRNA signature; and
(c) A method of identifying an miRNA signature associated with a specific disease or pathological state of a subject infected with a virus, comprising generating a miRNA profile from a test sample obtained from the subject, and comparing the test sample miRNA profile to an appropriate control sample miRNA profile, wherein one or more alterations between the test sample miRNA profile and the control sample miRNA profile defines the miRNA signature associated with the disease or pathological state;
in a quantitative reverse-transcription polymerase chain reaction assay.

116. A kit comprising:
(a) a microarray comprising miRNA-specific probe oligonucleotides for the miRNAs of the miRNA signature identified according to a method of selected from the group consisting of:
(i) a method of identifying a virus-specific miRNA signature, comprising generating a miRNA profile from a test sample obtained from a cell infected with a virus and comparing the test sample a miRNA profile to an appropriate control sample miRNA profile, wherein one or more alterations between the test sample miRNA profile and the control sample miRNA profile defines the virus-specific miRNA signature;
(ii) a method of identifying a virus-specific, replication stage-specific miRNA signature, comprising generating a miRNA profile from a test sample obtained from a cell infected with a virus, wherein said virus is in a replication stage selected from the group consisting of productive, persistent and latent, and comparing the test sample a miRNA profile to an appropriate control sample miRNA profile, wherein one or more alterations between the test sample a miRNA profile and the control sample miRNA profile defines the virus-specific, replication stage-specific miRNA signature; and
(iii) A method of identifying an miRNA signature associated with a specific disease or pathological state of a subject infected with a virus, comprising generating a miRNA profile from a test sample obtained from the subject, and comparing the test sample miRNA profile to an appropriate control sample miRNA profile, wherein one or more alterations between the test sample miRNA profile and the control sample miRNA profile defines the miRNA signature associated with the disease or pathological state; and
(b) instructions for use
optionally including a control sample.

117. A kit comprising:
(a) a set of oligonucleotide primers designed to specifically amplify the miRNAs of the miRNA signature identified according to a method selected from the group consisting of:
(i) a method of identifying a virus-specific miRNA signature, comprising generating a miRNA profile from a test sample obtained from a cell infected with a virus and comparing the test sample a miRNA profile to an appropriate control sample miRNA profile, wherein one or more alterations between the test sample miRNA profile and the control sample miRNA profile defines the virus-specific miRNA signature;
(ii) a method of identifying a virus-specific, replication stage-specific miRNA signature, comprising generating a miRNA profile from a test sample obtained from a cell infected with a virus, wherein said virus is in a replication stage selected from the group consisting of productive, persistent and latent, and comparing the test sample a miRNA profile to an appropriate control sample miRNA profile, wherein one or more alterations between the test sample a miRNA profile and the control sample miRNA profile defines the virus-specific, replication stage-specific miRNA signature; and
(iii) A method of identifying an miRNA signature associated with a specific disease or pathological state of a subject infected with a virus, comprising generating a miRNA profile from a test sample obtained from the subject, and comparing the test sample miRNA profile to an appropriate control sample miRNA profile, wherein one or more alterations between the test sample miRNA profile and the control sample miRNA profile defines the miRNA signature associated with the disease or pathological state;
in a quantitative reverse-transcription polymerase chain reaction assay; and
(b) instructions for use
optionally including a control sample.

118. The method of claim 104, wherein one or more miRNAs are selected from the group consisting of hsa-miR-34c*, hsa-miR-19b***, hsa-miR-19a***, hsa-miR-186, hsa-miR-17-5p***, hsa-miR-147*, hsa-miR-135b*, and hsa-miR-132; and/or are selected from the group consisting of hsa-miR-98, hsa-miR-505*, hsa-miR-423, hsa-miR-324-3p, hsa-miR-30a-5p, hsa-miR-28, hsa-miR-25, hsa-miR-224, hsa-miR-193b*, hsa-miR-181b, and hsa-let-7i.

119. The method of claim 106, wherein one or more miRNAs are selected from the group consisting of hsa-miR-126, hsa-miR-134, hsa-miR-135b*, hsa-miR-146a, hsa-miR-182, hsa-miR-183, hsa-miR-194, hsa-miR-199b*, hsa-miR-212*, hsa-miR-215, hsa-miR-34c*, hsa-miR-361*, hsa-miR-362, hsa-miR-422b, hsa-miR-500, hsa-miR-500*, hsa-miR-502*, hsa-miR-532, and hsa-miR-660; and/or are selected from the group consisting of hsa-miR-100, hsa-miR-10a, hsa-miR-10a*, hsa-miR-125b, hsa-miR-127, hsa-miR-143, hsa-miR-145, hsa-miR-145*, hsa-miR-148b, hsa-miR-152, hsa-miR-154, hsa-miR-155, hsa-miR-181a, hsa-miR-181b, hsa-miR-181c, hsa-miR-181d, hsa-miR-193a*, hsa-miR-199a, hsa-miR-199a*, hsa-miR-199b, hsa-miR-214, hsa-miR-218, hsa-miR-221*, hsa-miR-222, hsa-miR-29b-1*, hsa-miR-29b-2*, hsa-miR-335, hsa-miR-34a, hsa-miR-379*, hsa-miR-409-3p, hsa-miR-409-5p, hsa-miR-410, hsa-miR-411, hsa-miR-421, hsa-miR-424, hsa-miR-424*, hsa-miR-432, hsa-miR-450, hsa-miR-455, hsa-miR-484, hsa-miR-485-5p, hsa-miR-487b, hsa-miR-490*, hsa-miR-493-3p, hsa-miR-493-5p, hsa-miR-494, hsa-miR-495, hsa-miR-503, hsa-miR-505, hsa-miR-542-3p, hsa-miR-542-5p, hsa-miR-574, hsa-miR-594, hsa-miR-7, and hsa-miR-99a.
